(19) United States
(12) Patent Application Publication
(10) Pub. No.: US 2007/0043011 A1
(43) Pub. Date: Feb. 22, 2007

(54) Substituted benzanilide compound and noxious organism controlling agent

(76) Inventors: Eitatsu Ikeda, Funabashi-shi (JP); Kazuhige Maeda, Funabashi-shi (JP); Yuuki Furukawa, Funabashi-shi (JP); Shinji Takii, Minamisaitama-gun (JP)

Correspondence Address: MANELLI DENISON & SELTER, 2000 M STREET NW SUITE 700, WASHINGTON, DC, 20036-3307, US

(21) Appl. No.: 10/574,259

(22) PCT Filed: Sep. 30, 2004
(86) PCT No.: PCT/JP04/14810

§ 371 Date: Mar. 30, 2006

(30) Foreign Application Priority Data
Sep. 30, 2003 (JP) 2003-339358

(51) Int. Cl. A61K 31/53; A61K 31/501; A61K 31/505; A61K 31/4965; A61K 31/4439; A61K 31/433; A61K 31/4245; A61K 31/4178; A61K 31/4196; C07D 409/02; C07D 403/02

(52) U.S. Cl. 514/183; 514/242; 514/247; 514/256; 514/255.06; 514/241; 514/362; 514/364; 514/363; 514/357; 514/381; 514/383; 514/396; 514/317; 514/616; 544/179; 544/182; 544/212; 544/238; 544/329; 544/405; 544/406; 546/229; 546/336; 548/128; 548/131; 548/134; 548/253; 548/265.8

(57) ABSTRACT

The present invention is to provide a novel agrigultural chemical, particularly an insecticide or acaricide,
and discloses a substituted benzanilide compound represented by the formula (1): 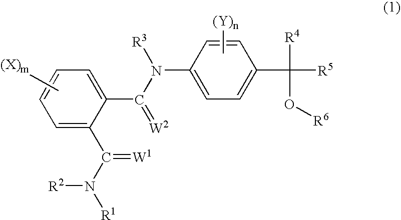
wherein W¹ and W² each independently represent an oxygen atom or a sulfur atom, X represents a halogen atom, etc., Y represents C₁ to C₆ alkyl, etc., R¹ represents C₁ to C₁₂ alkyl, a C₁ to C₆ alkylthio(C₁ to C₆) alkyl, a C₁ to C₆ alkylsulfinyl(C₁ to C₆) alkyl or a C₁ to C₆ alkylsulfonyl(C₁ to C₆) alkyl, etc., R² and R³ each independently represent a hydrogen atom, etc., R⁴ represents a C₁ to C₆ alkyl or a C₁ to C₆ haloalkyl, etc., R⁵ represents a phenoxy(C₁ to C₆) haloalkyl substituted by (Z)ₚ₁, a phenyl(C₂ to C₆) alkenyl, substituted by (Z)ₚ₁ phenyl, a phenyl substituted by (Z)ₚ₁ or L, etc., R⁶ represents a hydrogen atom, a C₁ to C₁₂ alkyl, a C₁ to C₆ alkoxy(C₁ to C₆) alkyl or a C₁ to C₆ alkylcarbonyl, etc., L represents an aromatic heterocyclic ring such as L-1 to L-4, L-8 to L-13, L-15 to L-23, L-45 to L-52 or L-53, etc., Z represents a halogen atom, a C₁ to C₆ haloalkyl, a C₁ to C₆ haloalkoxy or a C₁ to C₆ haloalkoxy-(C₁ to C₆) haloalkoxy, etc., m and n each independently is an integer of 0 to 4, p1 is an integer or 1 to 5, or a salt thereof, and a noxious organism controlling agent containing these.

### TECHNICAL FIELD

The present invention relates to a novel substituted benzanilide compound and a salt thereof, and a noxious organism controlling agent which comprises the compound as an effective ingredient. The noxious organism controlling agent in the present invention means a noxious insect controlling agent objective to noxious arthropods in the agricultural and horticultural field or stock-raising and hygiene field (a medicine for animals or an insecticide for domestic or business purpose). Also, as the agricultural chemical in the present invention means an insecticide, an acaricide, a nematocide, a herbicide and a fungicide in the agricultural and horticultural field.

### BACKGROUND ART

Heretofore, it has been known that specific substituted benzanilide derivatives have cytokine production inhibiting activity, vasopressin antagonistic activity, etc., and have been used as a medicine (for example, see WO 98/024771A brochure, WO 99/051580A brochure and JP-A-2002-249473.). Also, it has been known that specific substituted benzanilide derivatives have insecticidal activity (for example, see EP-A-0919542, EP-A-1006107, WO 01/021576A brochure, WO 01/046124A brochure, JP-A-2001-335559, WO 02/062807A brochure, WO 02/094765A brochure, WO 2004/000796A brochure and WO 2004/018415A brochure.). However, there is no disclosure therein about the substituted benzanilide compounds according to the present invention.

### DISCLOSURE OF THE INVENTION

### Problems to be Solved by the Invention

Due to use of noxious organism controlling agents such as an insecticide and a fungicide for a long period of time, noxious insects have obtained resistivity thereto in recent years, so that prevention thereof by the conventionally used insecticides or fungicides becomes difficult. Also, a part of the known noxious organism controlling agents has high toxicity, or some of them are putting an ecological system in confusion due to their long residual activity. Under such a circumstance, it has been usually expected to develop a novel noxious organism controlling agent which has low toxicity and low remaining property.

### Means to Solve the Problems

The present inventors have conducted earnest studies to solve the above-mentioned problems, and as a result, they have found that the novel substituted benzanilide compound represented by the following formula (1) according to the present invention is an extremely useful compound which has an excellent noxious organism controlling activity, in particular, an insecticidal and acaricidal activity, and causing substantially no bad effect against non-target organisms such as mammals, fishes and useful insects, whereby they have accomplished the present invention.

That is, the present invention relates to the following [1] to [8].
[1] A substituted benzanilide compound represented by the formula (1):

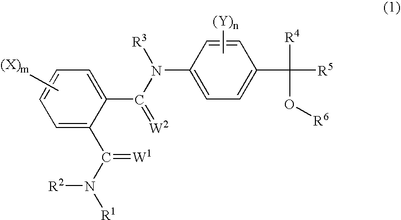

wherein W¹ and W² each independently represent an oxygen atom or a sulfur atom,

X represents a halogen atom, cyano, nitro, azide, —SCN, —SF₅, a C₁ to C₆ alkyl, a (C₁ to C₆) alkyl optionally substituted by R⁷, a C₃ to C₈ cycloalkyl, a (C₃ to C₈) cycloalkyl optionally substituted by R⁷, a C₂ to C₆ alkenyl, a (C₂ to C₆) alkenyl optionally substituted by R⁷, a C₃ to C₈ cycloalkenyl, a C₃ to C₈ halocycloalkenyl, a C₂ to C₆ alkynyl, a (C₂ to C₆) alkynyl optionally substituted by R⁷, —OH, —OR⁸, —OS(O)₂R⁸, —SH, S(O)ᵣR⁸, —CHO, —C(O)R⁹, —C(O)OR⁹, —C(O)SR⁹, —C(O)NHR¹⁰, —C(O)N(R¹⁰)R⁹, —C(S)OR⁹, —C(S)SR⁹, —C(S)NHR¹⁰, —C(S)N(R¹⁰)R⁹, —CH═NOR¹¹, —C(R⁹)═NOR¹¹, —S(O)₂OR⁹, —S(O)₂NHR¹⁰, —S(O)₂N(R¹⁰)R⁹, —Si(R¹³)(R¹⁴)R¹², phenyl, a phenyl substituted by (Z)ₚ₁, L or M, when m is 2, 3 or 4, each X may be the same or different from each other,

and when two Xs are adjacent to each other, the adjacent two Xs may form a 5-membered ring or 6-membered ring with the carbon atoms to which two Xs are bonded by forming —CH₂CH₂CH₂—, —CH₂CH₂O—, —CH₂OCH₂—, —OCH₂O—, —CH₂CH₂S—, —CH₂SCH₂—, —CH₂CH₂N(R¹⁵)—, —CH₂N(R¹⁵)CH₂—, —CH₂CH₂CH₂CH₂—, —CH₂CH₂CH₂O—, —CH₂CH₂OCH₂—, —CH₂OCH₂O—, —OCH₂CH₂O—, —OCH₂CH₂S—, —CH₂CH═CH—, —OCH═CH—, —SCH═CH—, —N(R¹⁵)CH═CH—, —OCH═N—, —SCH═N—, —N(R¹⁵)CH═N—, —N(R¹⁵)N═CH—, —CH═CHCH═CH—, —OCH₂CH═CH—, —N═CHCH═CH—, —N═CHCH═N— or —N═CHN═CH—, and at this time, each hydrogen atom bonded to the respective carbon atoms which form the ring may be optionally substituted by Z, and further when it is substituted by two or more Zs at the same time, each Z may be the same or different from each other, Y represents a halogen atom, cyano, nitro, a C₁ to C₆ alkyl, a (C₁ to C₆) alkyl optionally substituted by R⁷, a C₃ to C₈ cycloalkyl, —OR⁸, —S(O)ᵣR⁸, —NH₂, a C₁ to C₆ alkylamino, a di(C₁ to C₆ alkyl)amino or —Si(R³)(R⁴)R¹², when n is 2, 3 or 4, each Y may be the same or different from each other, and when two Ys are adjacent to each other, the adjacent two Ys may form a 5-membered ring or 6-membered ring with the carbon atoms to which two Ys are bonded by forming —CH₂CH₂CH₂—, —CH₂CH₂O—, —CH₂OCH₂—, —OCH₂O—, —CH₂CH₂S—, —CH₂SCH₂—, —SCH₂S—, —CH₂CH₂CH₂CH₂—, —CH₂CH₂CH₂O—, —CH₂CH₂OCH₂—, —CH₂OCH₂O—, —OCH₂CH₂O—, —OCH₂CH₂S—, —SCH₂CH₂S—, —OCH═N— or —SCH═N—, and at this time, each hydrogen atom bonded to the respective carbon atoms which form the ring may be optionally substituted by Z, and further when it is substituted by two or more Zs at the same time, each Z may be the same or different from each other,

R¹ represents a hydrogen atom, cyano, a C₁ to C₁₂ alkyl, a (C₁ to C₁₂) alkyl optionally substituted by R¹⁶, a C₃ to C₁₂ cycloalkyl, a (C₃ to C₁₂) cycloalkyl optionally substituted by R¹⁶, a C₃ to C₁₂ alkenyl, a (C₃ to C₁₂) alkenyl optionally substituted by R¹⁶, a C₃ to C₁₂ cycloalkenyl, a C₃ to C₁₂ halocycloalkenyl, a C₃ to C₁₂ alkynyl, a (C₃ to C₁₂) alkynyl optionally substituted by R¹⁶, —OH, a C₁ to C₈ alkoxy, a C₃ to C₈ alkenyloxy, a C₃ to C₈ haloalkenyloxy, phenoxy, a phenoxy substituted by (Z)ₚ₁, a phenyl(C₁ to C₄) alkoxy, a phenyl(C₁ to C₄) alkoxy substituted by (Z)ₚ₁, —N(R²⁰)R¹⁹, phenyl, a phenyl substituted by (Z)ₚ₁, L or M,

R² and R³ each independently represent a hydrogen atom, cyano, a C₁ to C₁₂ alkyl, a (C₁ to C₁₂) alkyl optionally substituted by R¹⁶, a C₃ to C₁₂ alkenyl, a C₃ to C₁₂ haloalkenyl, a C₃ to C₁₂ alkynyl, a C₃ to C₁₂ haloalkynyl, —OH, a C₁ to C₈ alkoxy, a C₁ to C₆ alkylthio, a C₁ to C₆ haloalkylthio, phenylthio, a phenylthio substituted by (Z)ₚ₁, —S(O)₂R⁹, —SN(R¹⁸)R¹⁷, —S(O)₂N(R¹⁰)R⁹, —N(R²⁰)R¹⁹, —C(O)R⁹, —C(O)OR⁹, —C(O)SR⁹, —C(O)N(R¹⁰)R⁹, —C(S)OR⁹, —C(S)SR⁹, —C(S)N(R¹⁰)R⁹, phenyl or a phenyl substituted by (Z)ₚ₁, or R² is combined with R¹ to form a C₂ to C₆ alkylene chain whereby it may form a 3 to 7-membered ring with the nitrogen atom to which they are bonded, and the alkylene chain at this time may contain one oxygen atom, sulfur atom or nitrogen atom, and may be optionally substituted by a halogen atom, a C₁ to C₆ alkyl group, a C₁ to C₆ haloalkyl group, a C₁ to C₆ alkoxy group, a C₁ to C₆ alkylcarbonyl group or a C₁ to C₆ alkoxycarbonyl group,

R⁴ represents a C₁ to C₆ alkyl, a C₁ to C₆ haloalkyl, a (C₁ to C₆) alkyl optionally substituted by R²¹, a (C₁ to C₆) haloalkyl optionally substituted by R²¹, a C₃ to C₈ cycloalkyl, a C₃ to C₈ halocycloalkyl, a (C₃ to C₈) cycloalkyl optionally substituted by R²¹, a (C₃ to C₈) halocycloalkyl optionally substituted by R²¹, a C₃ to C₆ alkenyl, a C₃ to C₆ haloalkenyl, a C₃ to C₆ alkynyl, a C₃ to C₈ haloalkynyl, phenyl, a phenyl substituted by (Z)ₚ₁, L or M,

R⁵ represents cyano, a (C₁ to C₆) alkyl optionally substituted by R²¹, a (C₁ to C₆) haloalkyl optionally substituted by R²¹, a (C₃ to C₈) cycloalkyl optionally substituted by R²¹, a (C₃ to C₈) halocycloalkyl optionally substituted by R²¹, a (C₂ to C₆) alkenyl optionally substituted by R²¹, a C₃ to C₈ cycloalkenyl, a C₃ to C₈ halocycloalkenyl, a (C₂ to C₆) alkynyl optionally substituted by R²¹, —OR⁸, —S(O)ᵣR⁸, —N(R¹⁰)R⁹, —CHO, —C(O)R⁹, —CH═NOR¹¹, —C(R⁹)═NOR¹¹, —C(O)OR⁹, —C(O)SR⁹, —C(O)NHR¹⁰, —C(O)N(R¹⁰)R⁹, —C(S)OR⁹, —C(S)SR⁹, —C(S)NHR¹⁰, —C(S)N(R¹⁰)R⁹, phenyl, a phenyl substituted by (Z)ₚ₁, biphenyl, a biphenyl substituted by (Z)ₚ₁, phenoxyphenyl, a phenoxyphenyl substituted by (Z)ₚ₁, pyridyloxyphenyl, a pyridyloxyphenyl substituted by (Z)ₚ₁, phenylthiophenyl, a phenylthiophenyl substituted by (Z)ₚ₁, phenylsulfinylphenyl, a phenylsulfinylphenyl substituted by (Z)ₚ₁, phenylsulfonylphenyl, a phenylsulfonylphenyl substituted by (Z)ₚ₁, L or M, or it forms a C₂ to C₃ alkylene chain with Y present at the adjacent position in combination whereby it may form a 5 to 6-membered ring which fuses with a benzene ring, and the alkylene chain at this time may contain one oxygen atom, sulfur atom or nitrogen atom, and may be optionally substituted by a halogen atom or a C₁ to C₆ haloalkyl group,

R⁶ represents a hydrogen atom, a C₁ to C₆ alkyl, a C₁ to C₆ haloalkyl, a (C₁ to C₆) alkyl optionally substituted by R²¹, a (C₁ to C₆) haloalkyl optionally substituted by R²¹, a C₃ to C₈ cycloalkyl, a C₃ to C₆ alkenyl, a C₃ to C₆ haloalkenyl, a phenyl(C₃ to C₆) alkenyl, a phenyl(C₃ to C₆) alkenyl substituted by (Z)ₚ₁, a C₃ to C₈ cycloalkenyl, a C₃ to C₆ alkynyl, a C₃ to C₆ haloalkynyl, a phenyl(C₃ to C₆) alkynyl, a phenyl(C₃ to C₆) alkynyl substituted by (Z)ₚ₁, —S(O)₂R⁹, —C(O)R⁹, —C(O)OR⁹, —C(O)SR⁹, —C(S)OR⁹, —C(S)SR⁹, —C(O)NHR¹⁰, —C(O)N(R¹⁰)R⁹, —C(S)NHR¹⁰, —C(S)N(R¹⁰)R⁹, —Si(R¹³)(R¹⁴)R¹², —P(O)(OR²²)₂, —P(S)(OR²²)₂ or M,

L represents an aromatic heterocyclic ring represented by any one of the formula L-1 to the formula L-58,

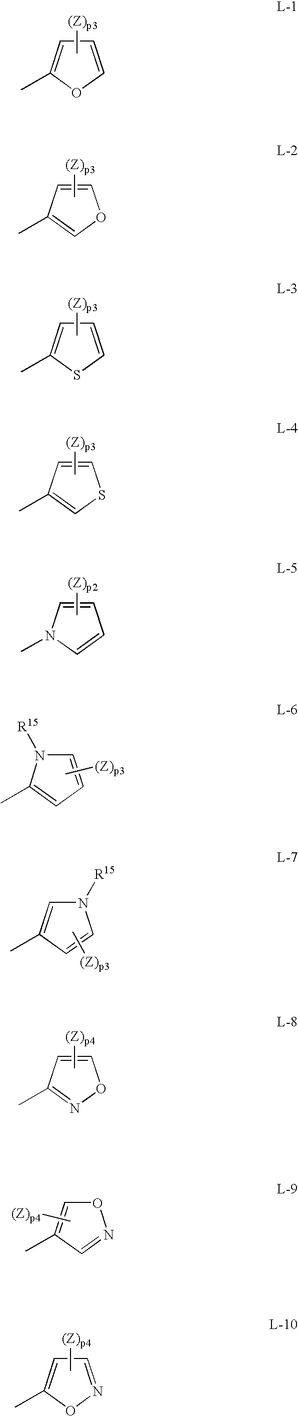

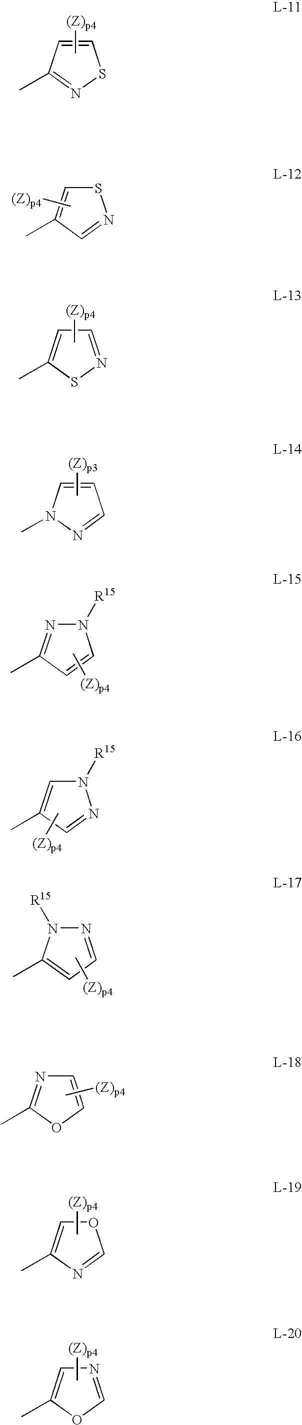

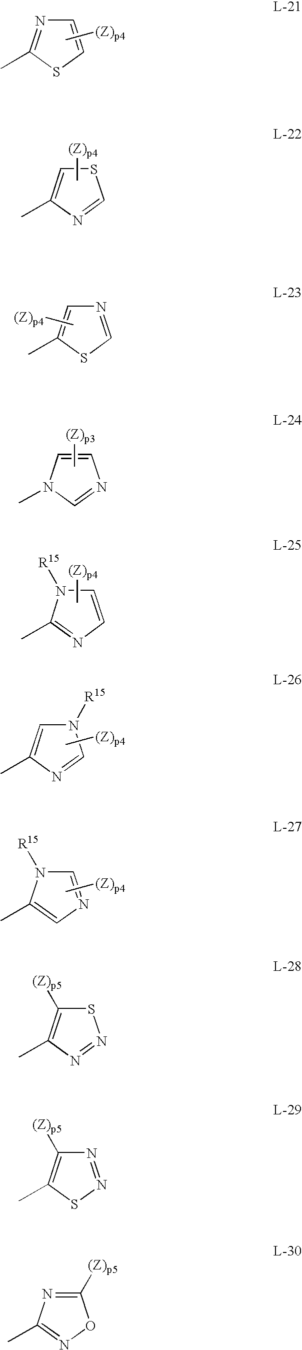

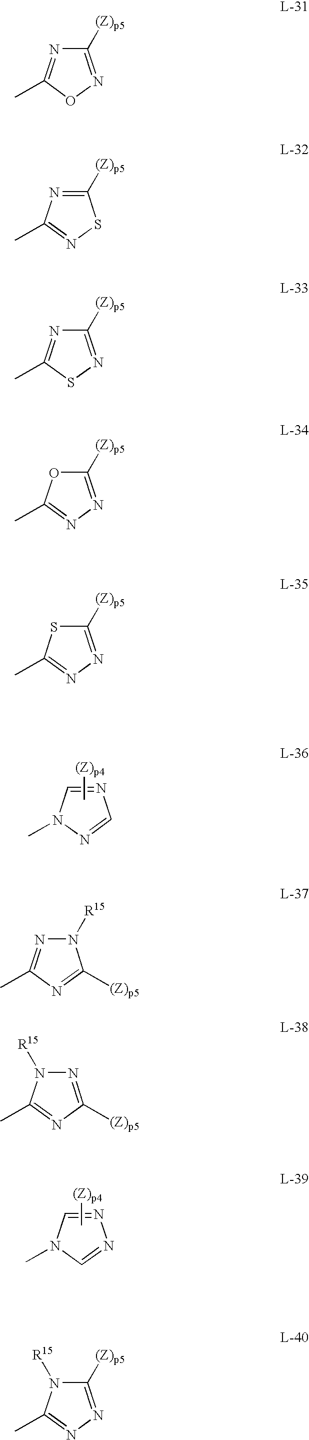

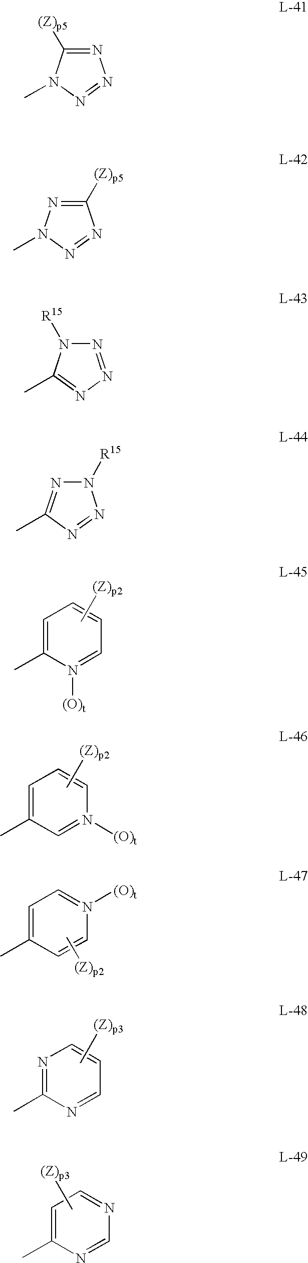

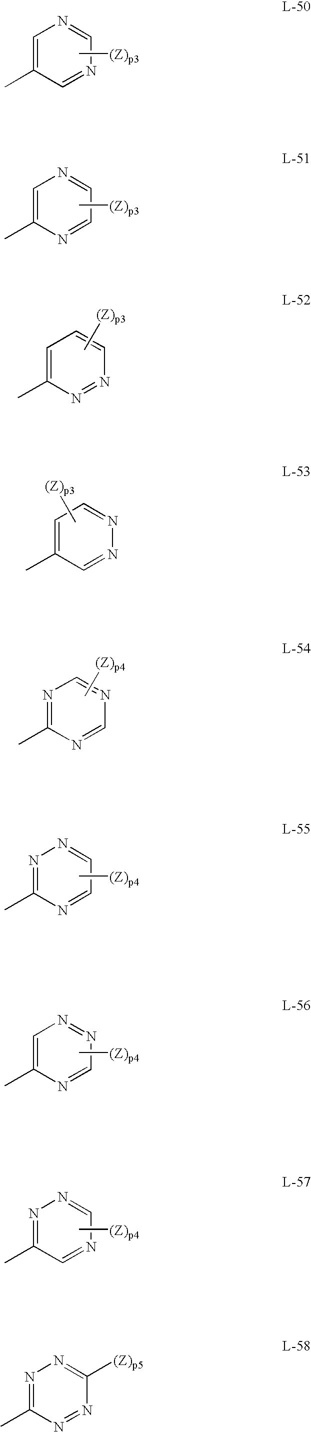

M represents an aromatic heterocyclic ring represented by any one of the formula M-1 to the formula M-28,

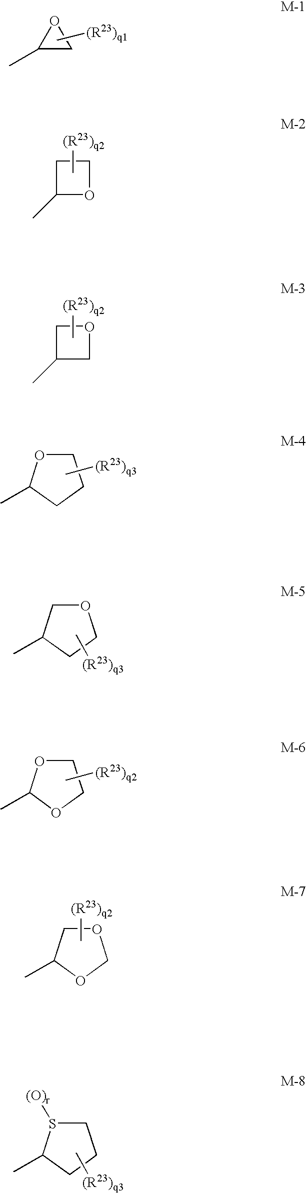

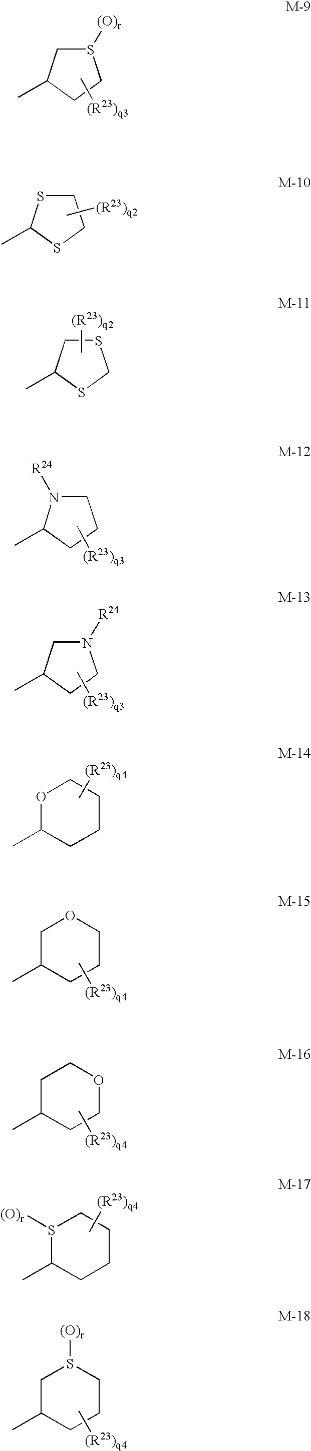

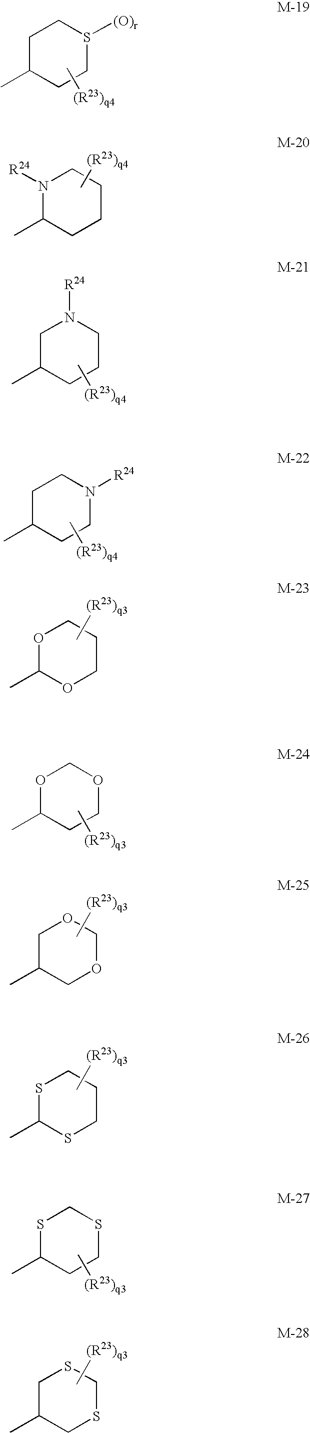

Z represents a halogen atom, cyano, nitro, azide, —SCN, —SF₅, a C₁ to C₆ alkyl, a C₁ to C₆ haloalkyl, a C₁ to C₃ alkoxy(C₁ to C₃)alkyl, a C₁ to C₃ haloalkoxy(C₁ to C₃)alkyl, cyano(C₁ to C₆)alkyl, hydroxy(C₁ to C₃) haloalkyl, a C₁ to C₃ alkoxy(C₁ to C₃) haloalkyl, a C₁ to C₃ haloalkoxy(C₁ to C₃) haloalkyl, a C₁ to C₃ alkylthio (C₁ to C₃)alkyl, a C₁ to C₃ haloalkylthio (C₁ to C₃)alkyl, a C₁ to C₃ alkylsulfinyl(C₁ to C₃)alkyl, a C₁ to C₃ haloalkylsulfinyl(C₁ to C₃)alkyl, a C₁ to C₃ alkylsulfonyl(C₁ to C₃)alkyl, a C₁ to C₃ haloalkylsulfonyl(C₁ to C₃)alkyl, a C₃ to C₈ cycloalkyl, a C₃ to C₈ halocycloalkyl, a C₂ to C₆ alkenyl, a C₂ to C₆ haloalkenyl, a C₃ to C₈ cycloalkenyl, a C₃ to C₈ halocycloalkenyl, a C₂ to C₆ alkynyl, a C₂ to C₆ haloalkynyl, —OH, a C₁ to C₆ alkoxy, a C₁ to C₆ haloalkoxy, a C₁ to C₃ haloalkoxy(C₁ to C₃) haloalkoxy, a C₂ to C₆ alkenyloxy, a C₂ to C₆ haloalkenyloxy, a C₃ to C₆ alkynyloxy, a C₃ to C₆ haloalkynyloxy, a C₁ to C₆ alkylsulfonyloxy, a C₁ to C₆ haloalkylsulfonyloxy, —SH, a C₁ to C₆ alkylthio, a C₁ to C₆ haloalkylthio, a C₁ to C₆ alkylsulfinyl, a C₁ to C₆ haloalkyl-sulfinyl, a C₁ to C₆ alkylsulfonyl, a C₁ to C₆ haloalkylsulfonyl, —NH₂, a C₁ to C₆ alkylamino, a di(C₁ to C₆ alkyl)amino, a C₁ to C₆ alkylsulfonylamino, a C₁ to C₆ haloalkylsulfonylamino, a C₁ to C₆ alkoxycarbonyl, a C₁ to C₆ haloalkoxycarbonyl, —C(O)NH₂, a C₁ to C₆ alkyl-aminocarbonyl, a di(C₁ to C₆ alkyl)aminocarbonyl, —C(S)NH₂, a C₁ to C₆ alkylaminosulfonyl, a di(C₁ to C₆ alkyl)aminosulfonyl or tri(C₁ to C₆ alkyl)silyl, when p1, p2, p3 or p4 is an integer of 2 or more, each Z may be the same or different from each other, further, when two Zs are adjacent to each other, the adjacent two Zs may form a 5-membered ring or 6-membered ring with the carbon atoms to which two Zs are bonded by forming —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —OCH₂CH₂S— or —CH═CHCH═CH—, and at this time, each hydrogen atom bonded to the respective carbon atoms which form the ring may be optionally substituted by a halogen atom or a C₁ to C₆ alkyl group,

R⁷ represents a halogen atom, cyano, a C₃ to C₈ cycloalkyl, a C₃ to C₈ halocycloalkyl, —OH, —OR⁸, —SH, —S(O)ᵣR⁸, —N(R¹⁰)R⁹, —N(R¹⁰)CHO, —N(R¹⁰)C(O)R⁹, —N(R¹⁰)C(O)OR⁹, —N(R¹⁰)C(O)SR⁹, —N(R¹⁰)C(S)OR⁹, —N(R¹⁰)C(S)SR⁹, —N(R¹⁰)S(O)₂R⁹, —C(O)OR⁹, —C(O)N(R¹⁰)R⁹, —Si(R³)(R⁴)R¹², phenyl, a phenyl substituted by (Z)ₚ₁, L or M,

R⁸ represents a C₁ to C₆ alkyl, a (C₁ to C₆) alkyl optionally substituted by R²⁵, a C₃ to C₈ cycloalkyl, a (C₃ to C₈) cycloalkyl optionally substituted by R²⁵, a C₂ to C₆ alkenyl, a (C₂ to C₆) alkenyl optionally substituted by R²⁵, a C₃ to C₈ cycloalkenyl, a C₃ to C₈ halocycloalkenyl, a C₃ to C₆ alkynyl, a (C₃ to C₆) alkynyl optionally substituted by R²⁵, phenyl, a phenyl substituted by (Z)ₚ₁, L or M,

R⁹ represents a C₁ to C₆ alkyl, a C₁ to C₆ haloalkyl, a C₃ to C₆ cycloalkyl (C₁ to C₄)alkyl, a C₁ to C₆ alkoxy(C₁ to C₄)alkyl, a C₁ to C₆ alkylthio (C₁ to C₄)alkyl, a cyano(C₁ to C₆)alkyl, a phenyl(C₁ to C₄)alkyl, a phenyl(C₁ to C₄) alkyl substituted by (Z)ₚ₁, an L-(C₁ to C₄)alkyl, an M-(C₁ to C₄)alkyl, a C₃ to C₈ cycloalkyl, a C₃ to C₈ halocycloalkyl, a C₃ to C₆ alkenyl, a C₃ to C₆ haloalkenyl, a C₃ to C₆ alkynyl, phenyl or a phenyl substituted by (Z)ₚ₁,

R¹⁰ represents a hydrogen atom or a C₁ to C₆ alkyl, or R⁹ and R¹⁰ are combined in combination to form a C₂ to C₆ alkylene chain whereby they may form a 3 to 7-membered ring with an atom(s) to which they are bonded, and at this time, the alkylene chain may contain one oxygen atom, sulfur atom or nitrogen atom, and may be optionally substituted by a halogen atom, a C₁ to C₆ alkyl group, a C₁ to C₆ alkoxy group, a formyl group, a C₁ to C₆ alkylcarbonyl group or a C₁ to C₆ alkoxycarbonyl group,

R¹¹ represents a hydrogen atom, a C₁ to C₆ alkyl, a C₁ to C₆ haloalkyl, a phenyl(C₁ to C₄)alkyl, a phenyl(C₁ to C₄) alkyl substituted by (Z)ₚ₁, a C₃ to C₆ alkenyl, a C₃ to C₆ haloalkenyl, a C₃ to C₆ alkynyl or a C₃ to C₆ haloalkynyl, or R¹¹ is combined with R⁹ to form a C₂ to C₄ alkylene chain whereby it may form a 5 to 7-membered ring with an atom(s) to which they are bonded, and at this time, the alkylene chain may contain one oxygen atom, sulfur atom or nitrogen atom, and may be optionally substituted by a halogen atom or a C₁ to C₆ alkyl group,

R¹² represents a C₁ to C₆ alkyl, a C₁ to C₆ haloalkyl, a C₃ to C₆ alkenyl, phenyl or a phenyl substituted by (Z)ₚ₁,

R¹³ and R¹⁴ each independently represent a C₁ to C₆ alkyl or a C₁ to C₆ haloalkyl,

R¹⁵ represents a hydrogen atom, a C₁ to C₆ alkyl, a C₁ to C₆ haloalkyl, a C₁ to C₆ alkoxycarbonyl(C₁ to C₄)alkyl, a C₁ to C₆ haloalkoxycarbonyl(C₁ to C₄)alkyl, a phenyl(C₁ to C₄)alkyl, a phenyl(C₁ to C₄) alkyl substituted by (Z)ₚ₁, a C₃ to C₆ alkenyl, a C₃ to C₆ haloalkenyl, a C₃ to C₆ alkynyl, a C₃ to C₆ haloalkynyl, a C₁ to C₆ alkoxy, a C₁ to C₆ alkoxycarbonyl, a C₁ to C₆ haloalkoxycarbonyl, phenyl or a phenyl substituted by (Z)ₚ₁,

R¹⁶ represents a halogen atom, cyano, nitro, a C₃ to C₈ cycloalkyl, a C₃ to C₈ halocycloalkyl, —OR²⁶, —N(R²⁷)R²⁶, —SH, —S(O)ᵣR²⁸—SO₂NHR³⁰, —SO₂N(R³⁰)R²⁹, —CHO, —C(O)R²⁹, —C(O)OH, —C(O)OR²⁹, —C(O)SR²⁹, —C(O)NHR³⁰, —C(O)N(R³⁰)R²⁹, —C(O)C(O)OR²⁹, —C(R³²)═NOH, —C(R³²)═NOR³¹, —Si(R¹³)(R¹⁴)R¹², —P(O)(OR²²)₂, —P(S)(OR²²)₂, —P(phenyl)₂, P(O)(phenyl)₂, phenyl, a phenyl substituted by (Z)ₚ₁, L or M,

R¹⁷ represents a C₁ to C₁₂ alkyl, a C₁ to C₁₂ haloalkyl, a C₁ to C₁₂ alkoxy(C₁ to C₁₂)alkyl, a cyano(C₁ to C₁₂)alkyl, a C₁ to C₁₂ alkoxycarbonyl(C₁ to C₁₂)alkyl, a phenyl(C₁ to C₄)alkyl, a phenyl(C₁ to C₄) alkyl substituted by (Z)ₚ₁, a C₃ to C₁₂ alkenyl, a C₃ to C₁₂ haloalkenyl, a C₃ to C₁₂ alkynyl, a C₃ to C₁₂ haloalkynyl, a C₁ to C₁₂ alkylcarbonyl, a C₁ to C₁₂ alkoxycarbonyl, phenyl or a phenyl substituted by (Z)ₚ₁,

R¹⁸ represents a C₁ to C₁₂ alkyl, a C₁ to C₁₂ haloalkyl, a C₁ to C₁₂ alkoxy(C₁ to C₁₂)alkyl, a cyano(C₁ to C₁₂)alkyl, a C₁ to C₁₂ alkoxycarbonyl(C₁ to C₁₂)alkyl, a phenyl(C₁ to C₄)alkyl, a phenyl(C₁ to C₄) alkyl substituted by (Z)ₚ₁, a C₃ to C₁₂ alkenyl, a C₃ to C₁₂ haloalkenyl, a C₃ to C₁₂ alkynyl, a C₃ to C₁₂ haloalkynyl, phenyl or a phenyl substituted by (Z)ₚ₁, or R¹⁷ and R¹⁸ are combined in combination to form a C₄ to C₇ alkylene chain whereby it may form a 5 to 8-membered ring with the nitrogen atom to which they are bonded, and at this time, the alkylene chain may contain one oxygen atom or sulfur atom, and may be optionally substituted by a C₁ to C₄ alkyl group or a C₁ to C₄ alkoxy group,

R¹⁹ represents a hydrogen atom, a C₁ to C₆ alkyl, a C₁ to C₆ haloalkyl, a phenyl(C₁ to C₄)alkyl, a phenyl(C₁ to C₄) alkyl substituted by (Z)ₚ₁, a C₃ to C₆ alkenyl, a C₃ to C₆ haloalkenyl, a C₃ to C₆ alkynyl, —CHO, a C₁ to C₆ alkylcarbonyl, a C₁ to C₆ haloalkylcarbonyl, a C₁ to C₆ alkoxycarbonyl, a C₁ to C₆ haloalkoxycarbonyl, a phenyl(C₁ to C₄) alkoxycarbonyl, a phenyl(C₁ to C₄) alkoxycarbonyl substituted by (Z)ₚ₁, phenoxycarbonyl, a phenoxycarbonyl substituted by (Z)ₚ₁, phenylcarbonyl, a phenylcarbonyl substituted by (Z)ₚ₁, phenyl or a phenyl substituted by (Z)ₚ₁,

R²⁰ represents a hydrogen atom, a C₁ to C₆ alkyl, —CHO, a C₁ to C₆ alkylcarbonyl, a C₁ to C₆ haloalkylcarbonyl or a C₁ to C₆ alkoxycarbonyl,

R²¹ represents cyano, a C₃ to C₈ cycloalkyl, a C₃ to C₈ halocycloalkyl, —OH, —OR⁸, —SH, —S(O)ᵣR⁸, —N(R¹⁰)R⁹, —N(R¹⁰)CHO, —N(R¹⁰)C(O)R⁹, —N(R¹⁰)C(O)OR⁹, —N(R¹⁰)C(O)SR⁹, —N(R¹⁰)C(S)OR⁹, —N(R¹⁰)C(S)SR⁹, —N(R¹⁰)S(O)₂R⁹, —C(O)OR⁹, —C(O)N(R¹⁰)R⁹, —Si(R³)(R⁴)R¹², phenyl, a phenyl substituted by (Z)ₚ₁, L or M,

R²² represents a C₁ to C₆ alkyl or a C₁ to C₆ haloalkyl,

R²³ represents a halogen atom, cyano, a C₁ to C₆ alkyl, a C₁ to C₆ haloalkyl, a hydroxy(C₁ to C₆)alkyl, a C₁ to C₄ alkoxy(C₁ to C₄)alkyl, a C₁ to C₄ alkoxycarbonyl(C₁ to C₄)alkyl, a C₁ to C₆ alkoxy, a C₁ to C₆ alkoxycarbonyl, phenyl or a phenyl substituted by (Z)ₚ₁, when q1, q2, q3 or q4 is an integer of 2 or more, each R²³ may be the same or different from each other,

R²⁴ represents a hydrogen atom, a C₁ to C₆ alkyl, a C₁ to C₆ haloalkyl, —CHO, a C₁ to C₆ alkylcarbonyl, a C₁ to C₆ haloalkylcarbonyl, a phenyl(C₁ to C₄)alkylcarbonyl, a phenyl(C₁ to C₄)alkylcarbonyl substituted by (Z)ₚ₁, a C₁ to C₆ alkoxycarbonyl, a C₁ to C₆ haloalkoxycarbonyl, a phenyl(C₁ to C₄) alkoxycarbonyl, a phenyl(C₁ to C₄)alkoxycarbonyl substituted by (Z)ₚ₁, a C₁ to C₆ alkylthio carbonyl, a C₁ to C₆ alkoxythiocarbonyl, a C₁ to C₆ alkylaminocarbonyl, a di(C₁ to C₆ alkyl)aminocarbonyl, a C₁ to C₆ alkylaminothiocarbonyl, a di(C₁ to C₆ alkyl)aminothiocarbonyl, phenylcarbonyl, a phenylcarbonyl substituted by (Z)ₚ₁, a C₁ to C₆ alkylsulfonyl, a C₁ to C₆ haloalkylsulfonyl, phenylsulfonyl, a phenylsulfonyl substituted by (Z)ₚ₁, —P(O)(OR²²)₂ or —P(S)(OR²²)₂,

R²⁵ represents a halogen atom, cyano, a C₃ to C₈ cycloalkyl, a C₃ to C₈ halocycloalkyl, a C₁ to C₆ alkoxy, a C₁ to C₆ haloalkoxy, a C₁ to C₆ alkylthio, a C₁ to C₆ haloalkylthio, a C₁ to C₆ alkylsulfonyl, a C₁ to C₆ haloalkylsulfonyl, a C₁ to C₆ alkylamino, a di(C₁ to C₆ alkyl)amino, —CHO, a C₁ to C₆ alkylcarbonyl, a C₁ to C₆ haloalkylcarbonyl, a C₁ to C₆ alkoxycarbonyl, a C₁ to C₆ haloalkoxycarbonyl, —CH═NOR¹¹, —C(R⁹)═NOR¹¹, phenyl, a phenyl substituted by (Z)ₚ₁, L or M,

R²⁶ represents a hydrogen atom, a C₁ to C₈ alkyl, a (C₁ to C₈) alkyl optionally substituted by R³³, a C₃ to C₈ cycloalkyl, a (C₃ to C₈) cycloalkyl optionally substituted by R³³, a C₃ to C₈ alkenyl, a (C₃ to C₈) alkenyl optionally substituted by R³³, a C₃ to C₈ alkynyl, a (C₃ to C₈) alkynyl optionally substituted by R³³, —CHO, —C(O)R²⁹, —C(O)OR²⁹, —C(O)SR²⁹, —C(O)NHR³⁰, —C(O)N(R³⁰)R²⁹, —C(O)C(O)R²⁹, —C(O)C(O)OR²⁹, —C(S)R²⁹, —C(S)OR²⁹, C(S)SR²⁹, —C(S)NHR³⁰, —C(S)N(R³⁰)R²⁹, —S(O)R²⁹, —S(O)₂N(R³⁰)R²⁹, —Si(R¹³)(R⁴)R¹², P(O)(OR²²)₂, —P(S)(OR²²)₂, phenyl, a phenyl substituted by (Z)ₚ₁, L or M,

R²⁷ represents a hydrogen atom, a C₁ to C₆ alkyl, a C₁ to C₆ haloalkyl, a C₃ to C₆ cycloalkyl or a C₁ to C₆ alkoxy, or R²⁶ and R²⁷ are combined in combination to form a C₂ to C₅ alkylene chain whereby it forms a 3 to 6-membered ring with the nitrogen atom to which they are bonded, and at this time, the alkylene chain may contain one oxygen atom or sulfur atom, and may be substituted by a halogen atom, a C₁ to C₆ alkyl group, a C₁ to C₆ alkoxy group, a phenyl group or a phenyl group substituted by (Z)ₚ₁,

R²⁸ represents a C₁ to C₈ alkyl, a (C₁ to C₈) alkyl optionally substituted by R³³, a C₃ to C₈ cycloalkyl, a (C₃ to C₈) cycloalkyl optionally substituted by R³³, a C₃ to C₈ alkenyl, a (C₃ to C₈) alkenyl optionally substituted by R³³, a C₃ to C₈ alkynyl, a (C₃ to C₈) alkynyl optionally substituted by R³³, —SH, a C₁ to C₆ alkylthio, a C₁ to C₆ haloalkylthio, phenylthio, a phenylthio substituted by (Z)ₚ₁, —CHO, C(O)R²⁹, —C(O)OR²⁹, —C(O)SR²⁹, —C(O)NHR³⁰, —C(O)N(R³⁰)R²⁹, —C(O)C(O)R²⁹, —C(O)C(O)OR²⁹, —C(S)R²⁹, —C(S)OR²⁹, —C(S)SR²⁹, —C(S)NHR³⁰, —C(S)N(R³⁰)R²⁹, —P(O)(OR²²)₂, —P(S)(OR²²)₂, phenyl, a phenyl substituted by (Z)ₚ₁, L-18, L-21, L-25, L-30 to L-35, L-45, L-48, L-49 or M,

R²⁹ represents a C₁ to C₆ alkyl, a C₁ to C₆ haloalkyl, a C₃ to C₈ cycloalkyl (C₁ to C₄)alkyl, a C₁ to C₆ alkoxy(C₁ to C₄)alkyl, a C₁ to C₆ haloalkoxy(C₁ to C₄)alkyl, a C₁ to C₆ alkylthio (C₁ to C₄)alkyl, a C₁ to C₆ haloalkylthio (C₁ to C₄)alkyl, a C₁ to C₆ alkylsulfonyl(C₁ to C₄)alkyl, a C₁ to C₆ haloalkylsulfonyl(C₁ to C₄)alkyl, a cyano(C₁ to C₆)alkyl, a C₁ to C₆ alkylcarbonyl(C₁ to C₄)alkyl, a C₁ to C₆ haloalkylcarbonyl(C₁ to C₄)alkyl, a C₁ to C₆ alkoxycarbonyl(C₁ to C₄)alkyl, a di(C₁ to C₆ alkyl)aminocarbonyl(C₁ to C₄)alkyl, a tri(C₁ to C₄ alkyl)silyl (C₁ to C₄)alkyl, a phenyl(C₁ to C₄)alkyl, a phenyl(C₁ to C₄) alkyl substituted by (Z)ₚ₁, an L-(C₁ to C₄)alkyl, an M-(C₁ to C₄)alkyl, a C₃ to C₈ cycloalkyl, a C₃ to C₈ halocycloalkyl, a C₂ to C₆ alkenyl, a C₂ to C₆ haloalkenyl, a C₂ to C₆ alkynyl, a C₂ to C₆ haloalkynyl, phenyl, a phenyl substituted by (Z)ₚ₁, L or M,

R³⁰ represents a hydrogen atom, a C₁ to C₆ alkyl, a C₁ to C₆ haloalkyl, phenyl or a phenyl substituted by (Z)ₚ₁, or R²⁹ and R³⁰ are combined to form a C₂ to C₅ alkylene chain whereby it may form a 3 to 6-membered ring with the nitrogen atom to which they are bonded, and at this time, the alkylene chain may contain one oxygen atom, sulfur atom or nitrogen atom, and may be optionally substituted by a halogen atom, a C₁ to C₆ alkyl group, a C₁ to C₆ alkoxy group, a formyl group, a C₁ to C₆ alkylcarbonyl group, a C₁ to C₆ alkoxycarbonyl group, a phenyl group or a phenyl group substituted by (Z)ₚ₁,

R³¹ represents a hydrogen atom, a C₁ to C₈ alkyl, a (C₁ to C₈) alkyl optionally substituted by R³³, a C₃ to C₈ cycloalkyl, a C₃ to C₈ alkenyl, a (C₃ to C₈) alkenyl optionally substituted by R³³, a C₃ to C₈ alkynyl or a (C₃ to C₈) alkynyl optionally substituted by R³³,

R³² represents a hydrogen atom, a C₁ to C₆ alkyl, a C₁ to C₆ haloalkyl, a C₃ to C₈ cycloalkyl (C₁ to C₄)alkyl, a C₁ to C₆ alkoxy(C₁ to C₄)alkyl, a C₁ to C₆ haloalkoxy(C₁ to C₄)alkyl, a C₁ to C₆ alkylthio (C₁ to C₄)alkyl, a C₁ to C₆ haloalkylthio (C₁ to C₄)alkyl, a C₁ to C₆ alkylsulfonyl(C₁ to C₄)alkyl, a C₁ to C₆ haloalkylsulfonyl(C₁ to C₄)alkyl, a phenyl(C₁ to C₄)alkyl, a phenyl(C₁ to C₄) alkyl substituted by (Z)ₚ₁, phenyl or a phenyl substituted by (Z)ₚ₁,

R³³ represents a halogen atom, cyano, nitro, a C₃ to C₈ cycloalkyl, a C₃ to C₈ halocycloalkyl, —OH, —OR³⁴, —SH, —S(O)ᵣR³⁴, —NHR³⁵, —N(R³⁵)R³⁴, —CHO, —C(O)R²⁹, —C(O)OR²⁹, —C(O)SR²⁹, —C(O)NHR³⁰, —C(O)N(R³⁰)R²⁹, —C(O)C(O)OR²⁹, —CH═NOR¹¹, —C(R⁹)═NOR¹¹, —Si(R¹³)(R¹⁴)R¹², —P(O)(OR²²)₂, —P(S)(OR²²)₂, —P(phenyl)₂, —P(O)(phenyl)₂, phenyl, a phenyl substituted by (Z)ₚ₁, L or M,

R³⁴ represents a C₁ to C₆ alkyl, a C₁ to C₆ haloalkyl, a C₁ to C₆ alkoxy(C₁ to C₄)alkyl, a C₁ to C₆ alkylthio(C₁ to C₄)alkyl, a phenyl(C₁ to C₄)alkyl, a phenyl(C₁ to C₄) alkyl substituted by (Z)ₚ₁, a C₃ to C₈ cycloalkyl, a C₃ to C₈ halocycloalkyl, a C₃ to C₆ alkenyl, a C₃ to C₆ haloalkenyl, a C₃ to C₈ cycloalkenyl, a C₃ to C₈ halocycloalkenyl, a C₃ to C₆ alkynyl, a C₃ to C₆ haloalkynyl, —CHO, a C₁ to C₆ alkylcarbonyl, a C₁ to C₆ haloalkylcarbonyl, a C₁ to C₆ alkoxycarbonyl, a C₁ to C₆ haloalkoxycarbonyl, a C₁ to C₆ alkylaminocarbonyl, a di(C₁ to C₆ alkyl) aminocarbonyl, phenylcarbonyl, a phenylcarbonyl substituted by (Z)ₚ₁, a C₁ to C₆ alkylaminothiocarbonyl, a di(C₁ to C₆ alkyl)aminothiocarbonyl, phenyl, a phenyl substituted by (Z)ₚ₁, L or M,

R³⁵ represents a hydrogen atom, a C₁ to C₆ alkyl, a C₁ to C₆ haloalkyl, a C₃ to C₈ cycloalkyl, a C₃ to C₆ alkenyl, a C₃ to C₆ alkynyl, a C₁ to C₆ alkylcarbonyl, a C₁ to C₆ haloalkylcarbonyl, a C₁ to C₆ alkoxycarbonyl, a C₁ to C₆ haloalkoxycarbonyl, phenoxycarbonyl, a phenoxycarbonyl substituted by (Z)ₚ₁, phenylcarbonyl, a phenylcarbonyl substituted by (Z)ₚ₁, a C₁ to C₆ alkylsulfonyl, a C₁ to C₆ haloalkylsulfonyl, phenyl, a phenyl substituted by (Z)ₚ₁, L or M, or R³⁴ and R³⁵ are combined to form a C₂ to C₅ alkylene chain, whereby it may form a 3 to 6-membered ring with the nitrogen atom to which they are bonded, and at this time, the alkylene chain may contain one oxygen atom or sulfur atom, and may be substituted by a halogen atom or a methyl group,

m is an integer of 0 to 4,

n is an integer of 0 to 4,

p1 is an integer of 1 to 5,

p2 is an integer of 0 to 4,

p3 is an integer of 0 to 3,

p4 is an integer of 0 to 2,

p5 is an integer of 0 or 1,

q1 is an integer of 0 to 3,

q2 is an integer of 0 to 5,

q3 is an integer of 0 to 7,

q4 is an integer of 0 to 9,

r is an integer of 0 to 2,

t is an integer of 0 or 1,

or a salt thereof.

[2] The substituted benzanilide compound according to the above [1], wherein W¹ and W² each respresent an oxygen atom,

X represents a halogen atom, cyano, nitro, a C₁ to C₆ alkyl, a C₁ to C₆ haloalkyl, a C₁ to C₆ alkoxy, a C₁ to C₆ haloalkoxy, a C₁ to C₆ alkylsulfonyloxy, a C₁ to C₆ haloalkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl or a C₁ to C₆ haloalkylsulfonyl, and when m is 2 or 3, each X may be the same or different from each other, and when two Xs are adjacent to each other, the adjacent two Xs may form a 5-membered ring or 6-membered ring with the carbon atoms to which two Xs are bonded by forming —OCH₂O— or —OCH₂CH₂O—, and at this time, the hydrogen atom(s) bonded to the respective carbon atoms which form a ring may be optionally replaced with a halogen atom, a C₁ to C₄ alkyl group or a C₁ to C₄ haloalkyl group,

Y represents a halogen atom, a C₁ to C₆ alkyl, a C₁ to C₆ haloalkyl, a hydroxy(C₁ to C₆)alkyl, a C₁ to C₃ alkoxy(C₁ to C₃)alkyl, a C₁ to C₆ alkoxy, a C₁ to C₆ haloalkoxy, a C₁ to C₆ alkylthio, a C₁ to C₆ haloalkylthio, a C₁ to C₆ alkylamino or a di(C₁ to C₆ alkyl)amino, when n is 2 or 3, each Y may be the same or different from each other,

R¹ represents a C₁ to C₈ alkyl, a (C₁ to C₈) alkyl optionally substituted by R¹⁶, a C₃ to C₈ cycloalkyl, a C₃ to C₈ alkenyl, a C₃ to C₈ alkynyl, a C₁ to C₈ alkoxy, M-4, M-5, M-8, M-9, M-13 to M-19, M-21 or M-22,

R² and R³ each independently represent a hydrogen atom, a C₁ to C₆ alkyl, a C₁ to C₄ alkoxy(C₁ to C₄)alkyl, a C₁ to C₄ alkylthio (C₁ to C₄)alkyl, a C₁ to C₄ alkylsulfonyl(C₁ to C₄)alkyl, a C₃ to C₆ alkenyl, a C₃ to C₆ alkynyl, a C₁ to C₆ alkylthio, a C₁ to C₆ haloalkylthio, phenylthio, a phenylthio substituted by (Z)ₚ₁ or —SN(R¹⁸)R¹⁷, or R² and R¹ may be combined to form a C₂ to C₆ alkylene chain whereby they may form a 3 to 7-membered ring with the nitrogen atom to which they are bonded, and at this time, the alkylene chain may contain one oxygen atom or sulfur atom,

R⁴ represents a C₁ to C₆ alkyl, a C₁ to C₆ haloalkyl, a (C₁ to C₆) alkyl optionally substituted by R²¹, a (C₁ to C₆) haloalkyl optionally substituted by R²¹, a C₃ to C₈ cycloalkyl, a C₃ to C₈ halocycloalkyl, a C₃ to C₆ haloalkenyl, a C₃ to C₆ haloalkynyl, phenyl or a phenyl substituted by (Z)ₚ₁,

R⁵ represents cyano, a (C₁ to C₆) alkyl optionally substituted by R²¹, a (C₁ to C₆) haloalkyl optionally substituted by R²¹, a C₃ to C₈ cycloalkyl, a C₃ to C₈ halocycloalkyl, a (C₂ to C₆) alkenyl optionally substituted by R²¹, a (C₂ to C₆) alkynyl optionally substituted by R²¹, —C(O)OR⁹, —C(O)SR⁹, —C(O)NHR¹⁰, —C(O)N(R¹⁰)R⁹, —C(S)OR⁹, —C(S)SR⁹, —C(S)NHR¹⁰, —C(S)N(R¹⁰)R⁹, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-25 to L-35, L-37, L-38, L-40, L-43 to L-58, M-4, M-5, M-8, M-9, M-14 to M-18 or M-19, or may be combined with Y existing at the adjacent position to form a C₂ to C₃ alkylene chain, whereby it may form a 5 to 6-membered ring which fuses with a benzene ring, and at this time, the alkylene chain may contain one oxygen atom, sulfur atom or nitrogen atom, and may be optionally substituted by a halogen atom or a C₁ to C₆ haloalkyl group,

R⁶ represents a hydrogen atom, a C₁ to C₆ alkyl, a C₁ to C₆ haloalkyl, a C₁ to C₄ alkoxy(C₁ to C₄)alkyl, a C₁ to C₄ alkylthio(C₁ to C₄)alkyl, a cyano(C₁ to C₆)alkyl, a phenyl-(C₁ to C₄)alkyl, a phenyl(C₁ to C₄) alkyl substituted by (Z)ₚ₁, a C₃ to C₆ alkenyl, a C₃ to C₆ haloalkenyl, a phenyl(C₃ to C₆) alkenyl, a phenyl(C₃ to C₆) alkenyl substituted by (Z)ₚ₁, a C₃ to C₆ alkynyl, a C₃ to C₆ haloalkynyl, a phenyl(C₃ to C₆) alkynyl, a phenyl(C₃ to C₆) alkynyl substituted by (Z)ₚ₁, —S(O)₂R⁹, —C(O)R⁹, —C(O)NHR¹⁰, —C(O)N(R¹⁰)R⁹, —C(S)NHR¹⁰, —C(S)N(R¹⁰)R⁹, —Si(R¹³)(R¹⁴)R¹², —P(O)(OR²²) or —P(S)(OR²²)₂,

Z represents a halogen atom, cyano, nitro, a C₁ to C₆ alkyl, a C₁ to C₆ haloalkyl, a C₁ to C₃ alkylthio(C₁ to C₃)alkyl, a C₁ to C₃ haloalkylthio(C₁ to C₃)alkyl, a C₁ to C₃ alkylsulfinyl(C₁ to C₃)alkyl, a C₁ to C₃ haloalkylsulfinyl(C₁ to C₃)alkyl, a C₁ to C₃ alkylsulfonyl(C₁ to C₃)alkyl, a C₁ to C₃ haloalkylsulfonyl(C₁ to C₃)alkyl, a C₁ to C₆ alkoxy, a C₁ to C₆ haloalkoxy, a C₁ to C₃ haloalkoxy(C₁ to C₃) haloalkoxy, a C₁ to C₆ alkylsulfonyloxy, a C₁ to C₆ haloalkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl, a C₁ to C₆ haloalkylsulfonyl, —C(O)NH₂ or —C(S)NH₂, and when p1, p2, p3 or p4 is an integer of 2 or more, each Z may be the same or different from each other,

further, when two Zs are adjacent to each other, the adjacent two Zs may form a 5-membered ring or 6-membered ring with the carbon atoms to which two Zs are bonded by forming —CF₂CF₂O—, —CF₂OCF₂—, —OCF₂O—, —OCF₂CHFO—, —OCF₂CF₂O— or —CH═CHCH═CH—,

R⁹ represents a C₁ to C₆ alkyl, a C₁ to C₆ haloalkyl, a C₃ to C₆ cycloalkyl(C₁ to C₄)alkyl, a phenyl(C₁ to C₄)alkyl, a phenyl(C₁ to C₄) alkyl substituted by (Z)ₚ₁, a C₃ to C₈ cycloalkyl, a C₃ to C₈ halocycloalkyl, phenyl or a phenyl substituted by (Z)ₚ₁,

R¹⁰ represents a hydrogen atom or a C₁ to C₆ alkyl, or R⁹ and R¹⁰ are combined to form a C₄ to C₅ alkylene chain, whereby it may form a 5-membered ring or 6-membered ring with the nitrogen atom to which they are bonded, and at this time, the alkylene chain may contain one oxygen atom or sulfur atom,

R¹² represents a C₁ to C₆ alkyl, phenyl or a phenyl substituted by (Z)ₚ₁,

R¹³ and R¹⁴ each independently represent a C₁ to C₆ alkyl,

R¹⁵ represents a C₁ to C₆ alkyl, a C₁ to C₆ haloalkyl, a phenyl(C₁ to C₄)alkyl, a phenyl(C₁ to C₄) alkyl substituted by (Z)ₚ₁, phenyl or a phenyl substituted by (Z)ₚ₁,

R¹⁵ represents a halogen atom, cyano, a C₃ to C₆ cycloalkyl, —OR²⁶, —N(R²⁷)R²⁶, —S(O)ᵣR²⁸, —SO₂N(R³)R²⁹, a C₁ to C₆ alkoxycarbonyl, —C(O)N(R³⁰)R²⁹, —C(R³²)═NOH, —C(R³²)═NOR³¹, —Si(R¹³)(R⁴)R¹², phenyl, a phenyl substituted by (Z)ₚ₁, L-1, L-2, L-3, L-4, L-45, L-46, L-47 or M,

R¹⁷ represents a C₁ to C₆ alkyl, a C₁ to C₆ alkoxycarbonyl(C₁ to C₄) alkyl or a C₁ to C₆ alkoxycarbonyl,

R¹⁸ represents a C₁ to C₆ alkyl, or R¹⁷ and R¹⁸ are combined to form a C₄ to C₅ alkylene chain whereby it may form a 5-membered ring or 6-membered ring with the nitrogen atom to which they are bonded, and at this time, the alkylene chain may contain one oxygen atom or sulfur atom, and may be optionally substituted by a methyl group or a methoxy group,

R²¹ represents cyano, a C₃ to C₆ cycloalkyl, a C₃ to C₆ halocycloalkyl, a C₁ to C₆ alkoxy, a C₁ to C₆ haloalkoxy, phenoxy, a phenoxy substituted by (Z)ₚ₁, a C₁ to C₆ alkylthio, a C₁ to C₆ haloalkylthio, phenylthio, a phenylthio substituted by (Z)ₚ₁, a C₁ to C₆ alkylsulfinyl, a C₁ to C₆ haloalkylsulfinyl, a C₁ to C₆ alkylsulfonyl, a C₁ to C₆ haloalkylsulfonyl, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L -53,

R²² represents a C₁ to C₆ alkyl,

R²³ represents a C₁ to C₄ alkyl, when q1, q2, q3 or q4 is an integer of 2 or more, each R²³ may be the same or different from each other, R²⁴ represents —CHO, a C₁ to C₆ alkylcarbonyl, a C₁ to C₆ alkoxycarbonyl or a C₁ to C₆ alkylsulfonyl,

R²⁶ represents a hydrogen atom, a C₁ to C₆ alkyl, a C₁ to C₆ haloalkyl, a C₁ to C₄ alkoxy(C₁ to C₄)alkyl, a C₁ to C₄ alkylthio(C₁ to C₄)alkyl, a phenyl(C₁ to C₄)alkyl, a phenyl-(C₁ to C₄) alkyl substituted by (Z)ₚ₁, a C₁ to C₆ alkylcarbonyl, a C₁ to C₆ haloalkylcarbonyl, a C₃ to C₆ cycloalkylcarbonyl, a C₁ to C₆ alkoxycarbonyl, —C(O)N(R³⁰)R²⁹, a C₁ to C₆ alkylsulfonyl, a di(C₁ to C₆ alkyl)aminosulfonyl, phenylsulfonyl, a phenylsulfonyl substituted by (Z)ₚ₁, a di(C₁ to C₆ alkyl)phosphoryl, a di(C₁ to C₆ alkyl)thiophosphoryl, a tri(C₁ to C₄ alkyl)-silyl, phenyl or a phenyl substituted by (Z)ₚ₁,

R²⁷ represents a hydrogen atom or a C₁ to C₆ alkyl,

R²⁸ represents a C₁ to C₆ alkyl, a C₁ to C₆ haloalkyl, a hydroxy(C₁ to C₄)alkyl, a C₁ to C₄ alkoxy(C₁ to C₄)alkyl, a C₁ to C₄ alkylthio(C₁ to C₄)alkyl, a C₁ to C₄ alkylcarbonyl(C₁ to C₄)alkyl, a C₁ to C₄ alkoxycarbonyl(C₁ to C₄)alkyl, a C₁ to C₄ alkylaminocarbonyl(C₁ to C₄)alkyl, a di(C₁ to C₄ alkyl)aminocarbonyl(C₁ to C₄)alkyl, a tri(C₁ to C₄ alkyl)silyl (C₁ to C₄)alkyl, a phenyl(C₁ to C₄)alkyl, a phenyl(C₁ to C₄) alkyl substituted by (Z)ₚ₁, a C₃ to C₆ alkenyl, a C₃ to C₆ alkynyl, a C₁ to C₆ alkylthio, phenyl, a phenyl substituted by (Z)ₚ₁, L-21, L-35, L-45 or L-48,

R²⁹ represents a C₁ to C₆ alkyl, a C₁ to C₄ alkoxy(C₁ to C₄)alkyl, a C₁ to C₄ alkylthio(C₁ to C₄)alkyl, a phenyl(C₁ to C₄)alkyl, a phenyl(C₁ to C₄) alkyl substituted by (Z)ₚ₁, a C₃ to C₆ cycloalkyl, a C₃ to C₆ alkenyl, a C₂ to C₆ alkynyl, phenyl or a phenyl substituted by (Z)ₚ₁,

R³⁰ represents a hydrogen atom or a C₁ to C₆ alkyl, or R²⁹ and R³⁰ are combined to form a C₂ to C₅ alkylene chain, whereby it may form a 3 to 6-membered ring with the nitrogen atom to which they are bonded, and at this time, the alkylene chain may contain one oxygen atom or sulfur atom,

R³¹ represents a C₁ to C₆ alkyl, a phenyl(C₁ to C₄) alkyl or a phenyl(C₁ to C₄) alkyl substituted by (Z)ₚ₁,

R³² represents a hydrogen atom or a C₁ to C₆ alkyl,

m is an integer of 0 to 3,

n is an integer of 0 to 3,

q2, q3 and q4 are each independently an integer of 0 to 2

or a salt thereof.

[3] The substituted benzanilide compound according to the above [2], wherein X represents a halogen atom, nitro, a C₁ to C₆ alkyl, a C₁ to C₆ haloalkyl, a C₁ to C₆ alkoxy, a C₁ to C₆ haloalkoxy, a C₁ to C₆ alkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl or a C₁ to C₆ haloalkylsulfonyl, and when m is 2, each X may be the same or different from each other,

Y represents a halogen atom, a C₁ to C₆ alkyl, a C₁ to C₆ haloalkyl, a C₁ to C₆ alkoxy or a C₁ to C₆ alkylthio, and when n is 2, each Y may be the same or different from each other,

R¹ represents a C₁ to C₈ alkyl, a (C₁ to C₈) alkyl optionally substituted by R¹⁶, a C₃ to C₈ alkenyl or a C₃ to C₈ alkynyl,

R² represents a hydrogen atom or a C₁ to C₆ alkyl,

R³ represents a hydrogen atom,

R⁴ represents a C₁ to C₆ alkyl, a C₁ to C₆ haloalkyl, a C₁ to C₃ alkoxy(C₁ to C₃)— haloalkyl, a C₁ to C₃ alkylthio(C₁ to C₃)haloalkyl, a C₃ to C₆ cycloalkyl or a C₃ to C₆ halocycloalkyl,

R⁵ represents a (C₁ to C₆) alkyl optionally substituted by R²¹, a (C₁ to C₆) haloalkyl optionally substituted by R²¹, a (C₂ to C₆) alkenyl optionally substituted by R²¹, a (C₂ to C₆) alkynyl optionally substituted by R²¹, a C₁ to C₆ alkoxycarbonyl, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 or L-53, or may be combined with Y existing at the adjacent position to form a C₂ to C₃ alkylene chain, whereby it may form a 5 to 6-membered ring which fuses with a benzene ring, and at this time, the alkylene chain may contain one oxygen atom, sulfur atom or nitrogen atom, and may be optionally substituted by a halogen atom,

R⁶ represents a hydrogen atom, a C₁ to C₆ alkyl, a C₁ to C₄ alkoxy(C₁ to C₄)alkyl, a C₁ to C₆ alkylcarbonyl or a tri(C₁ to C₄ alkyl)silyl,

R¹⁶ represents —OR²⁶, —N(R²⁷)R²⁶, —S(O)ᵣR²⁸, —SO₂N(R³⁰)R²⁹, —C(R³²)═NOH or —C(R³²)═NOR³¹,

R²¹ represents a C₁ to C₆ alkoxy, a C₁ to C₆ haloalkoxy, phenoxy, a phenoxy substituted by (Z)ₚ₁, phenylthio, a phenylthio substituted by (Z)ₚ₁, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53,

R²⁶ represents a hydrogen atom, a C₁ to C₆ alkyl, a C₁ to C₆ alkylcarbonyl, a C₁ to C₆ alkoxycarbonyl, a C₁ to C₆ alkylaminocarbonyl, a di(C₁ to C₆ alkyl)aminocarbonyl or a C₁ to C₆ alkylsulfonyl,

R²⁸ represents a C₁ to C₆ alkyl,

R²⁹ represents a C₁ to C₆ alkyl,

R³⁰ represents a hydrogen atom or a C₁ to C₆ alkyl,

R³¹ represents a C₁ to C₆ alkyl,

R³² represents a hydrogen atom,

m is an integer of 0 to 2,

n is an integer of 0 to 2

or a salt thereof.

[4] The substituted benzanilide compound according to the above [3], wherein X represents a halogen atom, nitro, a C₁ to C₄ alkyl, a C₁ to C₄ haloalkyl, a C₁ to C₄ alkylthio, a C₁ to C₄ alkylsulfinyl or a C₁ to C₄ alkylsulfonyl, and when m is 2, each X may be the same or different from each other,

Y represents a halogen atom or a C₁ to C₄ alkyl, when n is 2, each Y may be the same or different from each other,

R¹ represents a C₁ to C₈ alkyl, a C₁ to C₄ alkylthio(C₁ to C₄)alkyl, a C₁ to C₄ alkylsulfinyl(C₁ to C₄) alkyl or a C₁ to C₄ alkylsulfonyl(C₁ to C₄)alkyl,

R² represents a hydrogen atom,

R⁴ represents a C₁ to C₆ alkyl or a C₁ to C₆ haloalkyl,

R⁵ represents phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 or L-53, R⁶ represents a hydrogen atom

or a salt thereof.

[5] An N-substituted phenyl-3-nitrophthalimide or substituted aniline represented by the formula (2) or the formula (3):

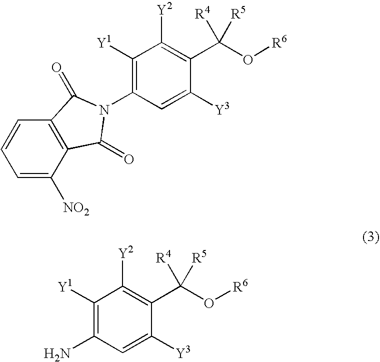

wherein Y¹ represents a hydrogen atom, a halogen atom, a C₁ to C₆ alkyl, a C₁ to C₆ haloalkyl, a C₁ to C₆ alkoxy or a C₁ to C₆ alkylthio,

Y² and Y³ each independently represent a hydrogen atom, or may form a C₂ to C₃ alkylene chain in combination with R⁵, whereby it may form a 5 to 6-membered ring which fuses with a benzene ring, at this time, the alkylene chain may contain one oxygen atom, sulfur atom or nitrogen atom, and may be optionally substituted by a halogen atom,

R⁴ represents a C₁ to C₆ alkyl, a C₁ to C₆ haloalkyl, a C₁ to C₃ alkoxy(C₁ to C₃) haloalkyl, a C₁ to C₃ alkylthio(C₁ to C₃) haloalkyl, a C₃ to C₆ cycloalkyl or a C₃ to C₆ halocycloalkyl,

R⁵ represents a (C₁ to C₆) alkyl optionally substituted by R²¹, a (C₁ to C₆) haloalkyl optionally substituted by R²¹, a (C₂ to C₆) alkenyl optionally substituted by R²¹, a (C₂ to C₆) alkynyl optionally substituted by R²¹, a C₁ to C₆ alkoxycarbonyl, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 or L-53,

R⁶ represents a hydrogen atom, a C₁ to C₆ alkyl, a C₁ to C₄ alkoxy(C₁ to C₄)alkyl, a C₁ to C₆ alkylcarbonyl or a tri(C₁ to C₄ alkyl)silyl,

R²¹ represents a C₁ to C₆ alkoxy, a C₁ to C₆ haloalkoxy, phenoxy, a phenoxy substituted by (Z)ₚ₁, phenylthio, a phenylthio substituted by (Z)ₚ₁, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53,

L-1 to L-5, L-8 to L-24, L-36, L-39, L-45 to L-52 or L-53 each represent the following aromatic heterocyclic ring,

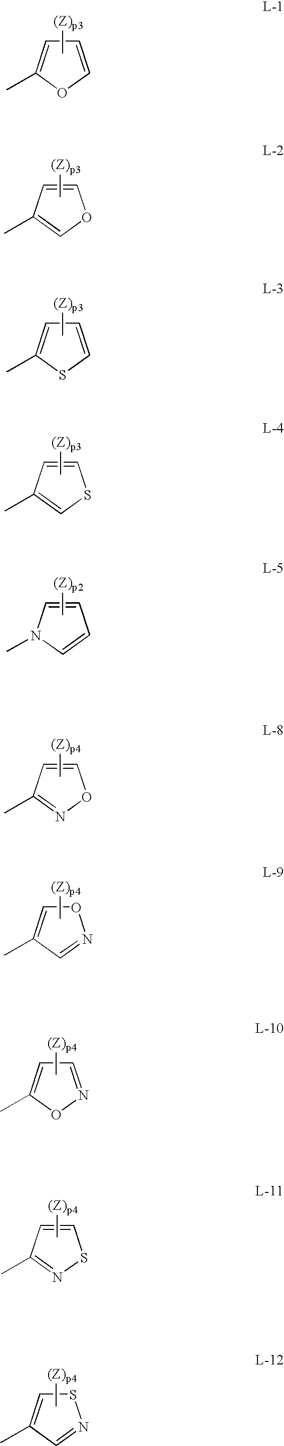

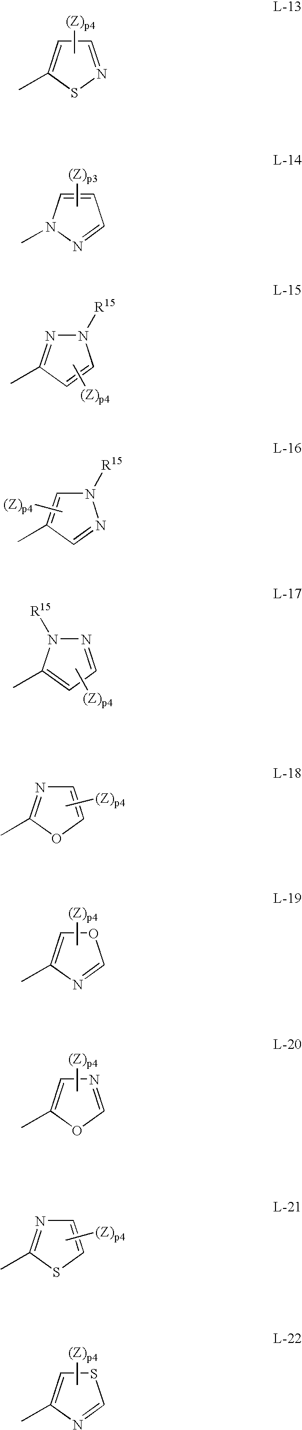

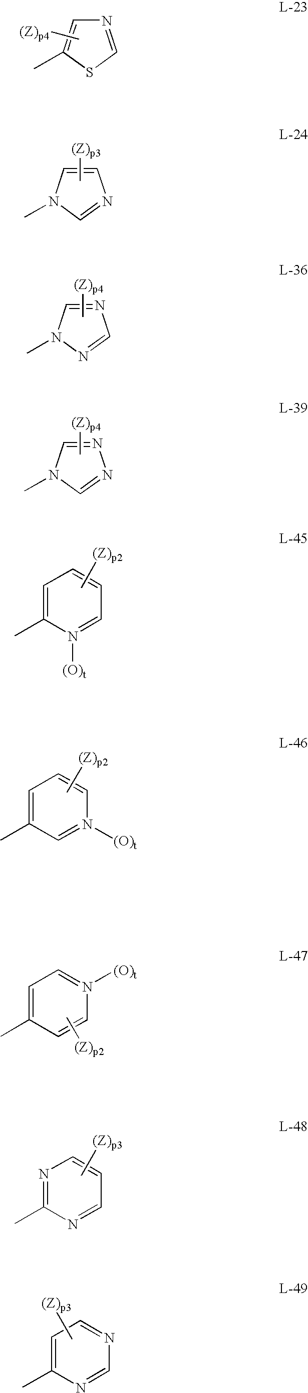

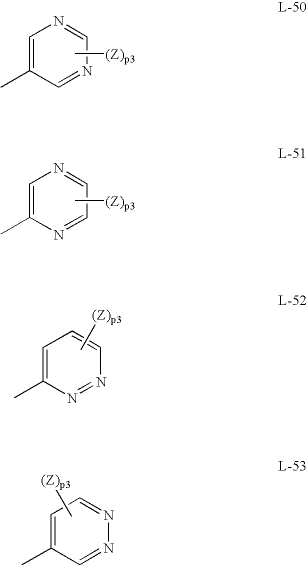

Z represents a halogen atom, cyano, nitro, a C₁ to C₆ alkyl, a C₁ to C₆ haloalkyl, a C₁ to C₃ alkylthio(C₁ to C₃)alkyl, a C₁ to C₃ haloalkylthio(C₁ to C₃)alkyl, a C₁ to C₃ alkylsulfinyl(C₁ to C₃)alkyl, a C₁ to C₃ haloalkylsulfinyl(C₁ to C₃)alkyl, a C₁ to C₃ alkylsulfonyl(C₁ to C₃)alkyl, a C₁ to C₃ haloalkylsulfonyl(C₁ to C₃)alkyl, a C₁ to C₆ alkoxy, a C₁ to C₆ haloalkoxy, a C₁ to C₃ haloalkoxy(C₁ to C₃) haloalkoxy, a C₁ to C₆ alkylsulfonyloxy, a C₁ to C₆ haloalkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl, a C₁ to C₆ haloalkylsulfonyl, —C(O)NH₂ or —C(S)NH₂, when p1, p2, p3 or p4 is an integer of 2 or more, each Z may be the same or different from each other,

further, when two Zs are adjacent to each other, the adjacent two Zs may form a 5-membered ring or 6-membered ring with the carbon atoms to which two Zs are bonded by forming —CF₂CF₂O—, —CF₂OCF₂—, —OCF₂O—, —OCF₂CHFO—, —OCF₂CF₂O— or —CH═CHCH═CH—,

R¹⁵ represents a C₁ to C₆ alkyl, phenyl or a phenyl substituted by (Z)ₚ₁,

p1 is an integer of 1 to 5,

p2 is an integer of 0 to 4,

p3 is an integer of 0 to 3,

p4 is an integer of 0 to 2,

p5 is an integer of 0 or 1,

r is an integer of 0 to 2,

t is an integer of 0 or 1.]

or a salt thereof.

[6] A noxious organism controlling agent which comprises one or more kinds selected from the substituted benzanilide compound and a salt thereof according to the above [1] to [4] as an effective ingredient.

[7] An agricultural chemical which comprises one or more kinds selected from the substituted benzanilide compound and a salt thereof according to the above [1] to [4] as an effective ingredient.

[8] An insecticide or araricide which comprises one or more kinds selected from the substituted benzanilide compound and a salt thereof according to the above [1] to [4] as an effective ingredient.

### Effects of the Invention

Due to use of an insecticide or a fungicide for a long period of time, noxious insects have obtained resistivity thereto in recent years, so that it becomes difficult to prevent them by the conventional insecticides or fungicides. Also, in a part of the insecticides, there exist those having high toxicity or having long residual activity in an environment, so that there is a problem that they disturb an ecological system. On the other hand, the compounds of the present invention have excellent insecticidal and acaricidal activities against many agricultural noxious insects and spider mites, and show sufficient preventing effects against noxious insects which obtained resistivity to the conventional insecticides. Moreover, the compounds do not substantially show bad influence against mammals, fishes and useful insects, and are low residual activity so that load against the environment is low.

Accordingly, the present invention can provide a useful and novel noxious organism controlling agent.

### BEST MODE FOR CARRYING OUT THE INVENTION

In the compounds included in the present invention, there are some cases in which geometric isomers of E-isomer and Z-isomer depending on the kind of the substituent(s), and the present invention includes these E-isomer, Z-isomer or a mixture of E-isomer and Z-isomer in an optional ratio. Also, in the compounds contained in the present invention, there exist optical isomers depending on the presence of one or more asymmetric carbon atoms, the present invention includes all the optical isomers or racemic mixtures. Moreover, in the compounds of the present invention represented by the formula (1), when R¹ or R² is a hydrogen atom, it can conceive the presence of tautomeric isomers represented by the following formula, and the present invention also includes these structures.

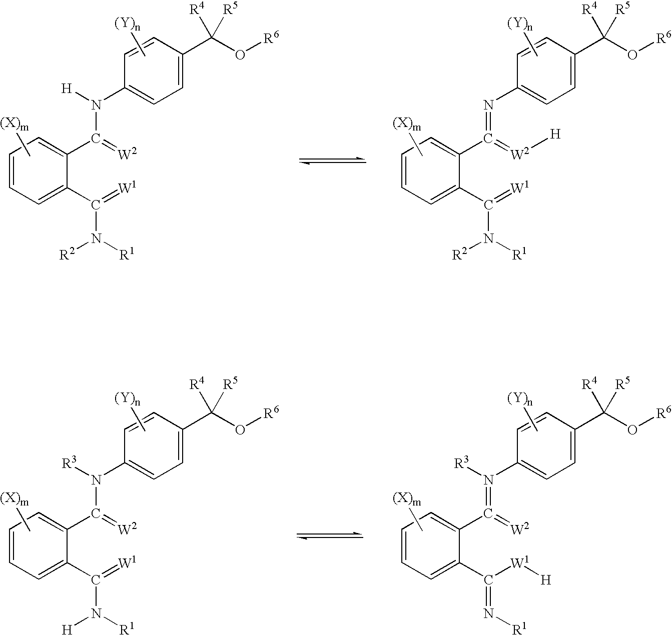

Among the compounds included in the present invention, those which can be an acid addition salt according to the conventional method may include, for example, a salt of a hydrohalogenic acid such as hydrofluoric acid, hydrochloric acid, hydrobromic acid, hydroiodic acid, etc., a salt of an inorganic acid such as nitric acid, sulfuric acid, phosphoric acid, chloric acid, perchloric acid, etc., a salt of a sulfonic acid such as methanesulfonic acid, ethanesulfonic acid, trifluoromethanesulfonic acid, benzene-sulfonic acid, p-toluenesulfonic acid, etc., a salt of a carbonic acid such as formic acid, acetic acid, propionic acid, trifluoroacetic acid, fumaric acid, tartaric acid, oxalic acid, maleic acid, malic acid, succinic acid, benzoic acid, mandelic acid, ascorbic acid, lactic acid, gluconic acid, citric acid, etc., or a salt of an amino acid such as glutamic acid, aspartic acid, etc.

Or else, among the compounds included in the present invention, those which can be made a metal salt according to the conventional manner may include, for example, a salt of an alkali metal such as lithium, sodium and potassium, a salt of an alkaline earth metal such as calcium, barium and magnesium or a salt of aluminum.

Next, specific examples of the respective substituent(s) shown in the present specification are shown below. Here, n—means normal, i—means iso, s—means secondary and t—means tertiary, respectively, and Ph means phenyl.

As the halogen atom in the compounds of the present invention, there may be mentioned a fluorine atom, a chlorine atom, a bromine atom and an iodine atom. Incidentally, the expression “halo” also means these halogen atoms in the present specification.

The expression Cₐ to C_{b} alkyl in the present specification represents a linear or branched hydrocarbon group having a number of a to b carbon atoms, and, for example, specific examples may include a methyl group, ethyl group, n-propyl group, i-propyl group, n-butyl group, s-butyl group, i-butyl group, t-butyl group, n-pentyl group, 1-methylbutyl group, 2-methylbutyl group, 3-methylbutyl group, 1-ethylpropyl group, 1,1-dimethylpropyl group, 1,2-dimethylpropyl group, neopentyl group, n-hexyl group, 1-methylpentyl group, 2-methylpentyl group, 3-methylpentyl group, 4-methylpentyl group, 1-ethylbutyl group, 2-ethylbutyl group, 1,1-dimethylbutyl group, 1,2-dimethylbutyl group, 1,3-dimethylbutyl group, 2,2-dimethylbutyl group, 2,3-dimethylbutyl group, 3,3-dimethylbutyl group, 1,1,2-trimethylpropyl group, 1,2,2-trimethylpropyl group, 1-ethyl-1-methylpropyl group, 1-ethyl-2-methylpropyl group, heptyl group, 1-methylhexyl group, 1,1-dimethylpentyl group, octyl group, 1-methylheptyl group, 1,1-dimethylhexyl group, nonyl group, 1-methyloctyl group, 1,1-dimethylheptyl group, decyl group, 1-methylnonyl group, undecyl group, 1-methyldecyl group, dodecyl group, 1-methylundecyl group, etc., and it can be selected from a designated range of carbon atoms.

The expression Cₐ to C_{b} haloalkyl in the present specification represents a linear or branched hydrocarbon group having a to b carbon atoms in which the hydrogen atom bonded to the carbon atom is optionally substituted by a halogen atom, and when it is substituted by 2 or more halogen atoms, these halogen atoms may be the same with each other or may be different from each other. Specific examples may include, for example, a fluoromethyl group, chloromethyl group, bromomethyl group, difluoromethyl group, dichloromethyl group, trifluoromethyl group, chlorodifluoromethyl group, trichloromethyl group, bromodifluoromethyl group, 2-fluoroethyl group, 1-chloroethyl group, 2-chloroethyl group, 1-bromoethyl group, 2-bromoethyl group, 2,2-difluoroethyl group, 1,2-dichloroethyl group, 2,2-dichloroethyl group, 2-bromo-2-chloroethyl group, 2,2,2-trifluoroethyl group, 2,2,2-trichloroethyl group, 1,1,2,2-tetrafluoroethyl group, 2-chloro-1,1,2-trifluoroethyl group, 2-bromo-1,1,2-trifluoroethyl group, pentafluoroethyl group, 2-chloro-1,1,2,2-tetrafluoroethyl group, 1-chloro-1,2,2,2-tetrafluoroethyl group, 2-bromo-1,1,2,2-tetrafluoroethyl group, 2,2-dichloro-1,1,2-trifluoroethyl group, 2,2,2-trichloro-1,1-difluoroethyl group, 1-chloropropyl group, 2-chloropropyl group, 3-chloropropyl group, 3-bromopropyl group, 2-fluoro-1-methylethyl group, 2-chloro-1-methylethyl group, 2-bromo-1-methylethyl group, 2,2,3,3,3-pentafluoropropyl group, 1,1,2,3,3,3-hexafluoropropyl group, 2,2,2-trifluoro-1-trifluoromethylethyl group, heptafluoropropyl group, 1,2,2,2-tetrafluoro-1-trifluoromethylethyl group, 2-bromo-1,1,2,3,3,3-hexafluoropropyl group, 4-chlorobutyl group, 2-chloro-1,1-dimethylethyl group, 2-bromo-1,1-dimethylethyl group, 3,3,3-trifluoro-1-methylpropyl group, nonafluorobutyl group, 5-chloropentyl group, 2,3-dibromo-1,1-dimethylpropyl group, 6-chlorohexyl group, tridecafluorohexyl group, 7-bromoheptyl group, 8-chlorooctyl group, 9-bromononyl group, 10-chlorodecyl group, 11-bromoundecyl group, 12-bromododecyl group, etc., and each may be selected from the range of the carbon numbers as designated, respectively.

The expression hydroxy(Cₐ to C_{b}) alkyl in the present specification represents a linear or branched alkyl group having a to b carbon atoms in which the hydrogen atom bonded to the carbon atom is optionally substituted by a hydroxyl group, and there may be specifically mentioned, for example, hydroxymethyl group, 1-hydroxyethyl group, 2-hydroxyethyl group, 3-hydroxypropyl group, 2-hydroxy-1-methylethyl group, 4-hydroxybutyl group, 2-hydroxy-1,1-dimethylethyl group, 3-hydroxy-1-methylpropyl group, 6-hydroxyhexyl group, etc., and each may be selected from the range of the carbon numbers as designated, respectively.

The expression cyano(Cₐ to C_{b}) alkyl in the present specification represents a linear or branched alkyl group having a to b carbon atoms in which the hydrogen atom bonded to the carbon atom is optionally substituted by a cyano group, and there may be specifically mentioned, for example, cyanomethyl group, 1-cyanoethyl group, 2-cyanoethyl group, 3-cyanopropyl group, 1-cyano-1-methylethyl group, 4-cyanobutyl group, 2-cyano-1,1-dimethylethyl group, 1-cyano-1-methylpropyl group, 6-cyanohexyl group, etc., and each may be selected from the range of the carbon numbers as designated, respectively.

The expression Cₐ to C_{b} cycloalkyl in the present specification represents a cyclic hydrocarbon group having a to b carbon atoms, and may form a monocyclic or heterocyclic structure from a 3-membered ring to a 6-membered ring. Also, respective rings may be optionally substituted by an alkyl group(s) in the range of the designated number of the carbon atoms. There may be specifically mentioned, for example, cyclopropyl group, 1-methylcyclopropyl group, 2-methylcyclopropyl group, 2,2-dimethylcyclopropyl group, 2,2,3,3-tetramethylcyclopropyl group, cyclobutyl group, cyclopentyl group, 1-methylcyclopentyl group, 2-methylcyclopentyl group, 3-methylcyclopentyl group, cyclohexyl group, 1-methylcyclohexyl group, 2-methylcyclohexyl group, 3-methylcyclohexyl group, 4-methylcyclohexyl group, bicyclo[2.2.1]heptan-2-yl group, etc., and each may be selected from the range of the carbon numbers as designated, respectively.

The expression Cₐ to C_{b} halocycloalkyl in the present specification represents a cyclic hydrocarbon group having a to b carbon atoms in which the hydrogen atom bonded to the carbon atom is optionally substituted by a halogen atom, and may form a monocyclic or heterocyclic structure from a 3-membered ring to a 6-membered ring. Also, respective rings may be optionally substituted by an alkyl group(s) in the range of the designated number of the carbon atoms, substitution by the halogen atom may be at the ring structure portion, a side chain portion, or may be both of the portions, and further, when it is substituted by 2 or more halogen atoms, these halogen atoms may be the same with each other or may be different from each other. There may be specifically mentioned, for example, 1-bromocyclopropyl group, 2,2-dichlorocyclopropyl group, 2,2-dibromocyclopropyl group, 2,2-difluoro-1-methylcyclopropyl group, 2,2-dichloro-1-methylcyclopropyl group, 2,2-dibromo-1-methylcyclopropyl group, 2,2-dichloro-3,3-dimethylcyclopropyl group, 2,2,3,3-tetrafluorocyclobutyl group, 2-fluorocyclohexyl group, 2-chlorocyclohexyl group, 3-chlorocyclohexyl group, 4-chlorocyclohexyl group, 2-trifluoromethylcyclohexyl group, 3-trifluoromethylcyclohexyl group, 4-trifluoromethylcyclohexyl group, etc., and each may be selected from the range of the carbon numbers as designated, respectively.

The expression Cₐ to C_{b} alkenyl in the present specification represents an unsaturated hydrocarbon group which is linear or branched having a to b carbon atoms, and having one or more double bonds in the molecule, and there may be specifically mentioned, for example, vinyl group, 1-propenyl group, 1-methylethenyl group, 2-propenyl group, 1-butenyl group, 1-methyl-1-propenyl group, 2-methyl-1-propenyl group, 2-butenyl group, 1-methyl-2-propenyl group, 2-methyl-2-propenyl group, 3-butenyl group, 1,3-butadienyl group, 1-methyl-2-butenyl group, 2-methyl-2-butenyl group, 3-methyl-2-butenyl group, 1,1-dimethyl-2-propenyl group, 2-hexenyl group, 2-methyl-2-pentenyl group, 1,3-dimethyl-2-butenyl group, 1,1,2-trimethyl-2-propenyl group, 1,1-dimethyl-3-butenyl group, 2,4-hexadienyl group, 2-heptenyl group, 1,1-dimethyl-4-pentenyl group, 2-octenyl group, 1-methyl-2-heptenyl group, 2-undecenyl group, 10-undecenyl group, 2-dodecenyl group, 11-dodecenyl group, etc., and each may be selected from the range of the carbon numbers as designated, respectively.

The expression Cₐ to C_{b} haloalkenyl in the present specification represents an unsaturated hydrocarbon group which is linear or branched having a to b carbon atoms, and having one or more double bonds in the molecule in which the hydrogen atom bonded to the carbon atom is optionally substituted by a halogen atom. At this time, when it is substituted by 2 or more halogen atoms, these halogen atoms may be the same with each other or may be different from each other. There may be specifically mentioned, for example, 2-chlorovinyl group, 2-bromovinyl group, 2,2-dichlorovinyl group, 2,2-dibromovinyl group, 3-bromo-2-propenyl group, 1-chloromethylvinyl group, 2-bromo-1-methylvinyl group, 1-trifluoromethylvinyl group, 2-chloro-3,3,3-trifluoro-1-propenyl group, 1-trifluoromethyl-2,2-difluorovinyl group, 2-chloro-2-propenyl group, 3,3-difluoro-2-propenyl group, 3,3-dichloro-2-propenyl group, 2,3,3-trifluoro-2-propenyl group, 2,3,3-trichloro-2-propenyl group, 4,4-difluoro-3-butenyl group, 3,4,4-trifluoro-3-butenyl group, 3-chloro-4,4,4-trifluoro-2-butenyl group, 3,3,3-trifluoro-1-methyl-1-propenyl group, 3,3,3-trifluoro-2-trifluoromethyl-1-propenyl group, 1,3,3,3-tetrafluoro-2-trifluoromethyl-1-propenyl group, 3,3,4,4,5,5,5-heptafluoro-1-pentenyl group, 5,5-difluoro-4-pentenyl group, 4,5,5-trifluoro-4-pentenyl group, 3,4,4,4-tetrafluoro-3-trifluoromethyl-1-butenyl group, 4,4,5,5,6,6,6-heptafluoro-2-hexenyl group, 3,4,4,5,5,5-hexafluoro -3-trifluoromethyl-1-pentenyl group, 2-perfluorohexylethenyl group, etc., and each may be selected from the range of the carbon numbers as designated, respectively.

The expression Cₐ to C_{b} cycloalkenyl in the present specification represents a cyclic unsaturated hydrocarbon group having a to b carbon atoms and having 1 or more double bonds, and may form a monocyclic or heterocyclic structure from a 3-membered ring to a 6-membered ring. Also, respective rings may be optionally substituted by an alkyl group(s) in the range of the designated number of the carbon atoms, and the double bond may be either of the endo- or exo-form. There may be specifically mentioned, for example, cyclopenten-1-yl group, 2-cyclopenten-1-yl group, 3-cyclopenten -1-yl group, cyclohexen-1-yl group, 2-cyclohexen-1-yl group, 3-cyclohexen-1-yl group, 2-methyl-2-cyclohexen-1-yl group, 3-methyl-2-cyclohexen-1-yl group, bicyclo-[2.2.1]-5-hepten-2-yl group, etc., and each may be selected from the range of the carbon numbers as designated, respectively.

The expression Cₐ to C_{b} halocycloalkenyl in the present specification represents a cyclic unsaturated hydrocarbon group having a to b carbon atoms and having 1 or more double bonds in which the hydrogen atom bonded to the carbon atom is optionally substituted by a halogen atom, and may form a monocyclic or heterocyclic structure from a 3-membered ring to a 6-membered ring. Also, the respective rings may be optionally substituted by an alkyl group(s) in the range of the designated number of the carbon atoms, and the double bond may be either of the endo- or exo-form. Also, substitution by the halogen atom may be at the ring structure portion, a side chain portion, or may be both of the portions, and further, when it is substituted by 2 or more halogen atoms, these halogen atoms may be the same with each other or may be different from each other. There may be specifically mentioned, for example, 2-chlorobicyclo[2.2.1]-5-hepten-2-yl group, etc., and each may be selected from the range of the carbon numbers as designated, respectively.

The expression Cₐ to C_{b} alkynyl in the present specification represents a linear or branched unsaturated hydrocarbon group having one or more triple bonds in the molecule with a to b carbon atoms, and there may be specifically mentioned, for example, ethynyl group, 1-propynyl group, 2-propynyl group, 1-methyl-2-propynyl group, 2-butynyl group, 3-butynyl group, 2-pentynyl group, 1-methyl-2-butynyl group, 1-methyl -3-butynyl group, 1,1-dimethyl-2-propynyl group, 1-hexynyl group, 3,3-dimethyl-1-butynyl group, 2-hexynyl group, 1-methyl-2-pentynyl group, 1,1-dimethyl-2-butynyl group, 2-heptynyl group, 1,1-dimethyl-2-pentynyl group, 2-octynyl group, 2-nonynyl group, 2-decynyl group, 2-undecynyl group, etc., and each may be selected from the range of the carbon numbers as designated, respectively.

The expression Cₐ to C_{b} haloalkynyl in the present specification represents a linear or branched unsaturated hydrocarbon group having one or more triple bonds in the molecule with a to b carbon atoms in which the hydrogen atom bonded to the carbon atom is optionally substituted by a halogen atom. At this time, when it is substituted by two or more halogen atoms, these halogen atoms may be the same with each other or may be different from each other. There may be specifically mentioned, for example, 2-chloroethynyl group, 2-bromoethynyl group, 2-iodoethynyl group, 3-chloro-2-propynyl group, 3-bromo-2-propynyl group, 3-iodo-2-propynyl group, 3,3,3-trifluoro-1-propynyl group, 3-chloro-1-methyl-2-propynyl group, 3-bromo-1-methyl-2-propynyl group, 3-iodo-1-methyl-2-propynyl group, 3-chloro-1,1-dimethyl-2-propynyl group, 3-bromo-1,1-dimethyl-2-propynyl group, 3-iodo-1,1-dimethyl-2-propynyl group, etc., and each may be selected from the range of the carbon numbers as designated, respectively.

The expression Cₐ to C_{b} alkoxy in the present specification represents an alkyl-O— group having the above-mentioned meaning with a to b carbon atoms, and there may be specifically mentioned, for example, methoxy group, ethoxy group, n-propyloxy group, i-propyloxy group, n-butyloxy group, s-butyloxy group, i-butyloxy group, t-butyloxy group, n-pentyloxy group, 1-methylbutyloxy group, 2-methylbutyloxy group, 3-methylbutyloxy group, 1-ethylpropyloxy group, 1,1-dimethylpropyloxy group, 1,2-dimethylpropyloxy group, neopentyloxy group, n-hexyloxy group, 1,1-dimethylbutyloxy group, etc., and each may be selected from the range of the carbon numbers as designated, respectively.

The expression Cₐ to C_{b} haloalkoxy in the present specification represents a haloalkyl-O— group having the above-mentioned meaning with a to b carbon atoms, and there may be specifically mentioned, for example, difluoromethoxy group, trifluoromethoxy group, chlorodifluoromethoxy group, bromodifluoromethoxy group, 2-fluoroethoxy group, 2-chloroethoxy group, 2,2,2-trifluoroethoxy group, 1,1,2,2,-tetrafluoroethoxy group, 2-chloro-1,1,2-trifluoroethoxy group, 2-bromo-1,1,2-trifluoroethoxy group, pentafluoroethoxy group, 2-bromo-1,1,2,2-tetrafluoroethoxy group, 2,2-dichloro-1,1,2-trifluoroethoxy group, 2,2,2-trichloro-1,1-difluoroethoxy group, 2-chloropropyloxy group, 3-chloropropyloxy group, heptafluoropropyloxy group, 2,2,2-trifluoro-1-trifluoromethylethoxy group, 2,2,3,3-tetrafluoropropyloxy group, 1,1,2,3,3,3-hexafluoropropyloxy group, 2-bromo-1,1,2,3,3,3-hexafluoropropyloxy group, etc., and each may be selected from the range of the carbon numbers as designated, respectively.

The expression Cₐ to C_{b} alkenyloxy in the present specification represents an alkenyl-O— group having the above-mentioned meaning with a to b carbon atoms, and there may be specifically mentioned, for example, 2-propenyloxy group, 2-butenyloxy group, 2-methyl-2-propenyloxy group, 3-methyl-2-butenyloxy group, etc., and each may be selected from the range of the carbon numbers as designated, respectively.

The expression Cₐ to C_{b} haloalkenyloxy in the present specification represents a haloalkenyl-O— group having the above-mentioned meaning with a to b carbon atoms, and there may be specifically mentioned, for example, 2-chloro-2-propenyl group, 3-chloro-2-propenyl group, 3,3-difluoro-2-propenyl group, 3,3-dichloro-2-propenyl group, 2,3,3-trifluoro-2-propenyl group, etc., and each may be selected from the range of the carbon numbers as designated, respectively.

The expression Cₐ to C_{b} alkylthio in the present specification represents an alkyl-S— group having the above-mentioned meaning with a to b carbon atoms, and there may be specifically mentioned, for example, methylthio group, ethylthio group, n-propylthio group, i-propylthio group, n-butylthio group, s-butylthio group, i-butylthio group, t-butylthio group, n-pentylthio group, 1-methylbutylthio group, 2-methylbutylthio group, 3-methylbutylthio group, 1-ethylpropylthio group, 1,1-dimethylpropylthio group, 1,2-dimethylpropylthio group, neopentylthio group, n-hexylthio group, etc., and each may be selected from the range of the carbon numbers as designated, respectively.

The expression Cₐ to C_{b} haloalkylthio in the present specification represents a haloalkyl-S— group having the above-mentioned meaning with a to b carbon atoms, and there may be specifically mentioned, for example, difluoromethylthio group, trifluoromethylthio group, bromodifluoromethylthio group, 2,2,2-trifluoroethylthio group, 1,1,2,2-tetrafluoroethylthio group, 1,1,2-trifluoro-2-chloroethylthio group, pentafluoroethylthio group, 2-bromo-1,1,2,2-tetrafluoroethylthio group, heptafluoropropylthio group, 1,2,2,2-tetrafluoro-1-trifluoromethylethylthio group, nonafluorobutylthio group, etc., and each may be selected from the range of the carbon numbers as designated, respectively.

The expression Cₐ to C_{b} alkylsulfinyl in the present specification represents an alkyl-S(O)— group having the above-mentioned meaning with a to b carbon atoms, and there may be specifically mentioned, for example, methylsulfinyl group, ethylsulfinyl group, n-propylsulfinyl group, i-propylsulfinyl group, n-butylsulfinyl group, s-butylsulfinyl group, i-butylsulfinyl group, t-butylsulfinyl group, etc., and each may be selected from the range of the carbon numbers as designated, respectively.

The expression Cₐ to C_{b} haloalkylsulfinyl in the present specification represents a haloalkyl-S(O)— group having the above-mentioned meaning with a to b carbon atoms, and there may be specifically mentioned, for example, difluoromethylsulfinyl group, trifluoromethylsulfinyl group, bromodifluoromethylsulfinyl group, 2,2,2-trifluoroethylsulfinyl group, 2-bromo-1,1,2,2-tetrafluoroethylsulfinyl group, 1,2,2,2-tetrafluoro-1-trifluoromethylethylsulfinyl group, nonafluorobutylsulfinyl group, etc., and each may be selected from the range of the carbon numbers as designated, respectively.

The expression Cₐ to C_{b} alkylsulfonyl in the present specification represents an alkyl-SO₂— group having the above-mentioned meaning with a to b carbon atoms, and there may be specifically mentioned, for example, methanesulfonyl group, ethanesulfonyl group, n-propylsulfonyl group, i-propylsulfonyl group, n-butylsulfonyl group, s-butylsulfonyl group, i-butylsulfonyl group, t-butylsulfonyl group, n-pentylsulfonyl group, n-hexylsulfonyl group, etc., and each may be selected from the range of the carbon numbers as designated, respectively.

The expression Cₐ to C_{b} haloalkylsulfonyl in the present specification represents a haloalkyl-SO₂— group having the above-mentioned meaning with a to b carbon atoms, and there may be specifically mentioned, for example, difluoromethanesulfonyl group, trifluoromethanesulfonyl group, chlorodifluoromethanesulfonyl group, bromodifluoromethanesulfonyl group, 2,2,2-trifluoroethanesulfonyl group, 1,1,2,2-tetrafluoroethanesulfonyl group, 1,1,2-trifluoro-2-chloroethanesulfonyl group, etc., and each may be selected from the range of the carbon numbers as designated, respectively.

The expression Cₐ to C_{b} alkylamino in the present specification represents an amino group in which either one of the hydrogen atoms is substituted by the alkyl group having the above-mentioned meaning with a to b carbon atoms, and there may be specifically mentioned, for example, methylamino group, ethylamino group, n-propylamino group, i-propylamino group, n-butylamino group, i-butylamino group, t-butylamino group, etc., and each may be selected from the range of the carbon numbers as designated, respectively.

The expression di(Cₐ to C_{b} alkyl)amino in the present specification represents an amino group in which both of the hydrogen atoms are substituted by the alkyl group having the above-mentioned meaning with a to b carbon atoms, which may be the same with each other or may be different from each other, and there may be specifically mentioned, for example, dimethylamino group, ethyl(methyl)amino group, diethylamino group, n-propyl(methyl)amino group, i-propyl(methyl)amino group, a di(n-propyl)amino group, n-butyl(methyl)amino group, i-butyl(methyl)amino group, t-butyl(methyl)amino group, etc., and each may be selected from the range of the carbon numbers as designated, respectively.

The expression Cₐ to C_{b} alkylcarbonyl in the present specification represents an alkyl-C(O)— group having the above-mentioned meaning with a to b carbon atoms, and there may be specifically mentioned, for example, CH₃C(O)— group, CH₃CH₂C(O)— group, a CH₃CH₂CH₂C(O)— group, (CH₃)₂CHC(O)— group, CH₃(CH₂)₃C(O)— group, (CH₃)₂CHCH₂C(O)— group, CH₃CH₂CH(CH₃)C(O)— group, (CH₃)₃CC(O)— group, CH₃(CH₂)₄C(O)— group, CH₃(CH₂)₅C(O)— group, etc., and each may be selected from the range of the carbon numbers as designated, respectively.

The expression Cₐ to C_{b} haloalkylcarbonyl in the present specification represents a haloalkyl-C(O)— group having the above-mentioned meaning with a to b carbon atoms, and there may be specifically mentioned, for example, FCH₂C(O)— group, ClCH₂C(O)— group, F₂CHC(O)— group, Cl₂CHC(O)— group, CF₃C(O)— group, ClCF₂C(O)— group, BrCF₂C(O)— group, CCl₃C(O)— group, CF₃CF₂C(O)— group, ClCH₂CH₂CH₂C(O)— group, CF₃CF₂CF₂C(O)— group, ClCH₂C(CH₃)₂C(O)— group, etc., and each may be selected from the range of the carbon numbers as designated, respectively.

The expression Cₐ to C_{b} cycloalkylcarbonyl in the present specification represents a cycloalkyl-C(O)— group having the above-mentioned meaning with a to b carbon atoms, and there may be specifically mentioned, for example, cyclopropyl-C(O)— group, 1-methylcyclopropyl-C(O)— group, 2-methylcyclopropyl-C(O)— group, 2,2-dimethylcyclopropyl-C(O)— group, cyclobutyl-C(O)— group, cyclopentyl-C(O)— group, cyclohexyl-C(O)— group, etc., and each may be selected from the range of the carbon numbers as designated, respectively.

The expression Cₐ to C_{b} alkoxycarbonyl in the present specification represents an alkyl-O—C(O)— group having the above-mentioned meaning with a to b carbon atoms, and there may be specifically mentioned, for example, CH₃OC(O)— group, CH₃CH₂OC(O)— group, CH₃CH₂CH₂OC(O)— group, (CH₃)₂CHOC(O)— group, CH₃(CH₂)₃OC(O)— group, (CH₃)₂CHCH₂OC(O)— group, (CH₃)₃COC(O)— group, etc., and each may be selected from the range of the carbon numbers as designated, respectively.

The expression Cₐ to C_{b} haloalkoxycarbonyl in the present specification represents a haloalkyl-O—C(O)— group having the above-mentioned meaning with a to b carbon atoms, and there may be specifically mentioned, for example, ClCH₂CH₂OC(O)— group, CF₃CH₂OC(O)— group, etc., and each may be selected from the range of the carbon numbers as designated, respectively.

The expression Cₐ to C_{b} alkylthiocarbonyl in the present specification represents an alkyl-S—C(O)— group having the above-mentioned meaning with a to b carbon atoms, and there may be specifically mentioned, for example, CH₃SC(O)— group, CH₃CH₂SC(O)— group, CH₃CH₂CH₂SC(O)— group, (CH₃)₂CHSC(O)— group, CH₃(CH₂)₃SC(O)— group, (CH₃)CHCH₂SC(O)— group, (CH₃)₃CSC(O)— group, etc., and each may be selected from the range of the carbon numbers as designated, respectively.

The expression Cₐ to C_{b} alkylthiocarbonyl in the present specification represents a alkyl-O—C(S)— group having the above-mentioned meaning with a to b carbon atoms, and there may be specifically mentioned, for example, CH₃SC(S)— group, CH₃CH₂SC(S)— group, CH₃CH₂CH₂SC(S)— group, (CH₃)₂CHOC(S)— group, CH₃(CH₂)₃SC(S)— group, (CH₃)CHCH₂SC(S)— group, (CH₃)₃COC(S)— group, etc., and each may be selected from the range of the carbon numbers as designated, respectively.

The expression Cₐ to C_{b} alkylaminocarbonyl in the present specification represents a carbamoyl group in which either one of the hydrogen atoms is substituted by the alkyl group having the above-mentioned meaning with a to b carbon atoms, and there may be specifically mentioned, for example, CH₃NHC(O)— group, CH₃CH₂NHC(O)— group, CH₃CH₂CH₂NHC(O)— group, (CH₃)₂CHNHC(O)— group, CH₃(CH₂)₃NHC(O)— group, (CH₃)₂CHCH₂NHC(O)— group, CH₃CH₂CH(CH₃)NHC(O)— group, (CH₃)₃CNHC(O)— group, etc., and each may be selected from the range of the carbon numbers as designated, respectively.

The expression di(Cₐ to C_{b} alkyl)aminocarbonyl in the present specification represents a carbamoyl group in which both of the hydrogen atoms are substituted by the alkyl group having the above-mentioned meaning with a to b carbon atoms, which may be the same with each other or may be different from each other, and there may be specifically mentioned, for example, (CH₃)₂NC(O)— group, CH₃CH₂N(CH₃)C(O)— group, (CH₃CH₂)₂NC(O)— group, (CH₃CH₂CH₂)₂NC(O)— group, (CH₃CH₂CH₂CH₂)₂NC(O)— group, etc., and each may be selected from the range of the carbon numbers as designated, respectively.

The expression Cₐ to C_{b} alkylaminothiocarbonyl in the present specification represents a thiocarbamoyl group in which either one of the hydrogen atoms is substituted by the alkyl group having the above-mentioned meaning with a to b carbon atoms, and there may be specifically mentioned, for example, CH₃NHC(S)— group, CH₃CH₂NHC(S)— group, CH₃CH₂CH₂NHC(S)— group, (CH₃)₂CHNHC(S)— group, CH₃(CH₂)₃NHC(S)— group, (CH₃)₂CHCH₂NHC(S)— group, CH₃CH₂CH(CH₃)NHC(S)— group, (CH₃)₃CNHC(S)— group, etc., and each may be selected from the range of the carbon numbers as designated, respectively.

The expression di(Cₐ to C_{b} alkyl)aminothiocarbonyl in the present specification represents a thiocarbamoyl group in which both of the hydrogen atoms are substituted by the alkyl group having the above-mentioned meaning with a to b carbon atoms, which may be the same with each other or may be different from each other, and there may be specifically mentioned, for example, (CH₃)₂NC(S)— group, CH₃CH₂N(CH₃)C(S)— group, (CH₃CH₂)₂NC(S)— group, (CH₃CH₂CH₂)₂NC(S)— group, (CH₃CH₂CH₂CH₂)₂NC(S)— group, etc., and each may be selected from the range of the carbon numbers as designated, respectively.

The expression Cₐ to C_{b} alkylaminosulfonyl in the present specification represents a sulfamoyl group in which either one of the hydrogen atoms is substituted by the alkyl group having the above-mentioned meaning with a to b carbon atoms, and there may be specifically mentioned, for example, CH₃NHSO₂— group, CH₃CH₂NHSO₂— group, CH₃CH₂CH₂NHSO₂— group, (CH₃)₂CHNHSO₂— group, CH₃(CH₂)₃NHSO₂— group, (CH₃)₂CHCH₂NHSO₂— group, CH₃CH₂CH(CH₃)NHSO₂— group, (CH₃)CNHSO₂— group, etc., and each may be selected from the range of the carbon numbers as designated, respectively.

The expression di(Cₐ to C_{b} alkyl)aminosulfonyl in the present specification represents a sulfamoyl group in which both of the hydrogen atoms are substituted by the alkyl group having the above-mentioned meaning with a to b carbon atoms, which may be the same with each other or may be different from each other, and there may be specifically mentioned, for example, (CH₃)₂NSO₂— group, CH₃CH₂N(CH₃)SO₂— group, (CH₃CH₂)₂NSO₂— group, (CH₃CH₂CH₂)₂NSO₂— group, (CH₃CH₂CH₂CH₂)₂NSO₂— group, etc., and each may be selected from the range of the carbon numbers as designated, respectively.

The expression di(Cₐ to C_{b} alkyl)phosphoryl in the present specification represents a phosphoryl group in which both of the hydrogen atoms are substituted by the alkyl group having the above-mentioned meaning with a to b carbon atoms, which may be the same with each other or may be different from each other, and there may be specifically mentioned, for example, (CH₃O)₂P(O)— group, (CH₃CH₂O)₂P(O)— group, etc., and each may be selected from the range of the carbon numbers as designated, respectively.

The expression di(Cₐ to C_{b} alkyl)thiophosphoryl in the present specification represents a thiophosphoryl group in which both of the hydrogen atoms are substituted by the alkyl group having the above-mentioned meaning with a to b carbon atoms, which may be the same with each other or may be different from each other, and there may be specifically mentioned, for example, (CH₃O)₂P(S)— group, (CH₃CH₂O)₂P(S)— group, etc., and each may be selected from the range of the carbon numbers as designated, respectively.

The expression tri(Cₐ to C_{b} alkyl)silyl in the present specification represents a silyl group which is substituted by the alkyl group(s) having the above-mentioned meaning with a to b carbon atoms, which may be the same with each other or may be different from each other, and there may be specifically mentioned, for example, trimethylsilyl group, triethylsilyl group, tri(n-propyl)silyl group, ethyldimethylsilyl group, n-propyldimethylsilyl group, n-butyldimethylsilyl group, i-butyidimethylsilyl group, t-butyldimethylsilyl group, etc., and each may be selected from the range of the carbon numbers as designated, respectively.

The expression Cₐ to C_{b} alkylsulfonyloxy in the present specification represents an alkyl-SO₂—O— group having the above-mentioned meaning with a to b carbon atoms, and there may be specifically mentioned, for example, CH₃SO₂—O— group, CH₃CH₂SO₂—O— group, CH₃CH₂CH₂SO₂—O— group, (CH₃)₂CHSO₂—O— group, CH₃(CH₂)₃SO₂—O— group, (CH₃)₂CHCH₂SO₂—O— group, CH₃CH₂CH(CH₃)SO₂—O— group, (CH₃)₃CSO₂—O— group, etc., and each may be selected from the range of the carbon numbers as designated, respectively.

The expression Cₐ to C_{b} haloalkylsulfonyloxy in the present specification represents a haloalkylsulfonyl-O— group having the above-mentioned meaning with a to b carbon atoms, and there may be specifically mentioned, for example, CF₃SO₂—O— group, CF₃CF₂SO₂—O— group, etc., and each may be selected from the range of the carbon numbers as designated, respectively.

The expression Cₐ to C_{b} alkylsulfonylamino in the present specification represents a alkylsulfonyl-NH— group having the above-mentioned meaning with a to b carbon atoms, and there may be specifically mentioned, for example, CH₃SO₂—NH— group, a CH₃CH₂SO₂—NH— group, a CH₃CH₂CH₂SO₂—NH— group, (CH₃)CHSO₂—NH— group, a CH₃(CH₂)₃SO₂—NH— group, (CH₃)₂CHCH₂SO₂—NH— group, a CH₃CH₂CH(CH₃)SO₂—NH— group, (CH₃)₃CSO₂—NH— group, etc., and each may be selected from the range of the carbon numbers as designated, respectively.

The expression Cₐ to C_{b} haloalkylsulfonylamino in the present specification represents a haloalkylsulfonyl-NH— group having the above-mentioned meaning with a to b carbon atoms, and there may be specifically mentioned, for example, CF₃SO₂—NH— group, a CF₃CF₂SO₂—NH— group, etc., and each may be selected from the range of the carbon numbers as designated, respectively.

The expression Cₐ to C_{b} cycloalkyl (C_{d} to Cₑ)alkyl, a Cₐ to C_{b} alkoxy(C_{d} to Cₑ)alkyl, a Cₐ to C_{b} haloalkoxy(C_{d} to Cₑ)alkyl, a Cₐ to C_{b} alkylthio (C_{d} to Cₑ)alkyl, a Cₐ to C_{b} haloalkylthio (C_{d} to Cₑ)alkyl, a Cₐ to C_{b} alkylsulfinyl(C_{d} to Cₑ)alkyl, a Cₐ to C_{b} haloalkylsulfinyl-(C_{d} to Cₑ)alkyl, a Cₐ to C_{b} alkylsulfonyl(C_{d} to Cₑ)alkyl, a Cₐ to C_{b} haloalkylsulfonyl(C_{d} to Cₑ)alkyl, a Cₐ to C_{b} alkylcarbonyl(C_{d} to Cₑ)alkyl, a Cₐ to C_{b} haloalkylcarbonyl(C_{d} to Cₑ)alkyl, a Cₐ to C_{b} alkoxycarbonyl(C_{d} to Cₑ)alkyl, a Cₐ to C_{b} haloalkoxycarbonyl(C_{d} to Cₑ)alkyl, a Cₐ to C_{b} alkylaminocarbonyl(C_{d} to Cₑ)alkyl, a di(Cₐ to C_{b} alkyl)aminocarbonyl(C_{d} to Cₑ)alkyl, tri(Cₐ to C_{b} alkyl)silyl (C_{d} to Cₑ)alkyl, a phenyl(C_{d} to Cₑ)alkyl, a phenyl(C_{d} to Cₑ) alkyl substituted by (Z)ₚ₁, L-(C_{d} to Cₑ) alkyl or M-(C_{d} to Cₑ)alkyl, etc., in the present specification each represent a linear or branched hydrocarbon group having d to e carbon atoms in which the hydrogen atom(s) bonded to the carbon atom(s) is/are optionally substituted by the optional Cₐ to C_{b}cyclo alkyl group, a Cₐ to C_{b} alkoxy group, a Cₐ to C_{b} haloalkoxy group, a Cₐ to C_{b} alkylthio group, a Cₐ to C_{b} haloalkylthio group, a Cₐ to C_{b} alkylsulfinyl group, a Cₐ to C_{b} haloalkylsulfinyl group, a Cₐ to C_{b} alkylsulfonyl group, a Cₐ to C_{b} haloalkylsulfonyl group, a Cₐ to C_{b} alkylcarbonyl group, a Cₐ to C_{b} haloalkylcarbonyl group, a Cₐ to C_{b} alkoxycarbonyl group, a Cₐ to C_{b} haloalkoxycarbonyl group, a Cₐ to C_{b} alkylaminocarbonyl group, a di(Cₐ to C_{b} alkyl)aminocarbonyl group, tri(Cₐ to C_{b} alkyl)silyl group, a phenyl group, a phenyl group substituted by (Z)ₚ₁, L group or M group, each of which have the above-mentioned meanings, and each may be selected from the range of the carbon numbers as designated, respectively.

The expression (Cₐ to C_{b}) alkyl optionally substituted by R⁷, (Cₐ to C_{b}) alkyl optionally substituted by R¹⁶, (Cₐ to C_{b}) alkyl optionally substituted by R²¹, (Cₐ to C_{b}) alkyl optionally substituted by R²⁵ or (Cₐ to C_{b}) alkyl optionally substituted by R³³ in the present specification represent a linear or branched hydrocarbon group having a to b carbon atoms in which the hydrogen atom(s) bonded to the carbon atom(s) is/are optionally substituted by an optional R⁷, R¹⁶, R²¹, R²⁵ or R³³, and each may be selected from the range of the carbon numbers as designated, respectively. At this time, when two or more substituent(s) R⁷, R¹⁶, R²¹, R²⁵ or R³³ exist on the respective (Cₐ to C_{b}) alkyl group, the respective R⁷, R¹⁶, R²¹, R²⁵ or R³³ may be the same with each other or may be different from each other.

The expression hydroxy(C_{d} to Cₑ)haloalkyl, Cₐ to C_{b} alkoxy(C_{d} to Cₑ)haloalkyl or Cₐ to C_{b} haloalkoxy(C_{d} to Cₑ)haloalkyl in the present specification represents a haloalkyl group having the above-mentioned meaning with d to e carbon atoms, optionally substituted by a hydroxyl group, a Cₐ to C_{b} alkoxy group having the above-mentioned meaning or a Cₐ to C_{b} haloalkoxy group having the above-mentioned meaning, and there may be specifically mentioned, for example, 2,2,2-trifluoro-1-hydroxy-1-(trifluoromethyl)-ethyl group, 2,2,2-trifluoro-1-methoxy-1-(trifluoromethyl)ethyl group, 2,2,2-trifluoro-1-(2,2,2-trifluoroethoxy)-1-(trifluoromethyl)ethyl group, 3-(1,2-dichloro-1,2,2-trifluoroethoxy)-1,1,2,2,3,3-hexafluoropropyl group, etc., and each may be selected from the range of the carbon numbers as designated, respectively.

The expression (Cₐ to C_{b}) haloalkyl optionally substituted by R²¹ in the present specification represents a haloalkyl group optionally substituted by R²¹ having the above-mentioned-meaning with a to b carbon atoms optionally substituted by R²¹, and each may be selected from the range of the carbon numbers as designated, respectively. At this time, when two or more substituent R²¹s exist on the respective (Cₐ to C_{b})haloalkyl group, the respective R²¹s may be the same with each other or may be different from each other.

The expression (Cₐ to C_{b}) cycloalkyl optionally substituted by R⁷, (Cₐ to C_{b}) cycloalkyl optionally substituted by R¹⁶, (Cₐ to C_{b}) cycloalkyl optionally substituted by R²¹, (Cₐ to C_{b}) cycloalkyl optionally substituted by R²⁵ or (Cₐ to C_{b}) cycloalkyl optionally substituted by R³³, etc., in the present specification represents a cycloalkyl group optionally substituted by R⁷, R¹⁶, R²¹, R²⁵ or R³³ having the above-mentioned meaning with a to b carbon atoms optionally substituted by R⁷, R¹⁶, R²¹, R²⁵ or R³³. At this time, substitution by R⁷, R¹⁶, R²¹, R²⁵ or R³³ may be at the ring structure portion or at the side chain portion, or may be at the both of them, and further, when two or more substituents R⁷s, R¹⁶s, R²¹s, R²⁵s or R³³s exist on the (Cₐ to C_{b}) cycloalkyl group, the respective R⁷s, R¹⁶s, R²¹s, R²⁵s or R³³s may be the same with each other or may be different from each other.

The expression (Cₐ to C_{b}) halocycloalkyl optionally substituted by R²¹ in the present specification represents a halocycloalkyl group optionally substituted by R²¹ having the above-mentioned meaning with a to b carbon atoms optionally substituted by R²¹. At this time, substitution by R²¹ may be at the ring structure portion or at the side chain portion, or may be at the both of them, and further, when two or more substituents R²¹ s exist on the respective (Cₐ to C_{b}) halocycloalkyl group, the respective R²¹s may be the same with each other or may be different from each other.

The expression phenyl(C_{d} to Cₑ)alkenyl or a phenyl(C_{d} to Cₑ)alkenyl substituted by (Z)ₚ₁, etc., in the present specification represents an alkenyl group having the above-mentioned meaning with d to e carbon atoms optionally substituted by a phenyl group or a phenyl group substituted by (Z)ₚ₁ each having the above-mentioned meanings, and each may be selected from the range of the carbon numbers as designated, respectively.

The expression (Cₐ to C_{b}) alkenyl optionally substituted by R⁷, a (Cₐ to C_{b}) alkenyl optionally substituted by R¹⁶, the (Cₐ to C_{b}) alkenyl optionally substituted by R²¹, the (Cₐ to C_{b}) alkenyl optionally substituted by R²⁵ or the (Cₐ to C_{b}) alkenyl optionally substituted by R³³ in the present specification represents an alkenyl group optionally substituted by R⁷, R¹⁶, R²¹, R²⁵ or R³³ having the above-mentioned meaning with a to b carbon atoms optionally substituted by R⁷, R¹⁶, R²¹, R²⁵ or R³³, and each may be selected from the range of the carbon numbers as designated, respectively. At this time, when two or more substituents R⁷s, R¹⁶s, R²¹s, R²⁵s or R³³s exist on the respective (Cₐ to C_{b}) alkenyl group, the respective R⁷s, R¹⁶s, R²¹s, R²⁵s or R³³s may be the same with each other or may be different from each other.

The expression phenyl(C_{d} to Cₑ) alkynyl or a phenyl(C_{d} to Cₑ) alkynyl substituted by (Z)ₚ₁ in the present specification represents an alkynyl group having the above-mentioned meaning with d to e carbon atoms optionally substituted by a phenyl group or a phenyl group substituted by (Z)ₚ₁, and each may be selected from the range of the carbon numbers as designated, respectively.

The expression (Cₐ to C_{b}) alkynyl optionally substituted by R⁷, the (Cₐ to C_{b}) alkynyl optionally substituted by R¹⁶, the (Cₐ to C_{b}) alkynyl optionally substituted by R²¹, the (Cₐ to C_{b}) alkynyl optionally substituted by R²⁵ or the (Cₐ to C_{b}) alkynyl optionally substituted by R³³ in the present specification represents an alkynyl group optionally substituted by R⁷, R¹⁶, R²¹, R²⁵ or R³³ having the above-mentioned meaning with a to b carbon atoms optionally substituted by R⁷, R¹⁶, R²¹, R²⁵ or R³³, and each may be selected from the range of the carbon numbers as designated, respectively. At this time, when two or more substituents R⁷s, R¹⁶s, R²¹s, R²⁵s or R³³s exist on the respective (Cₐ to C_{b}) alkynyl group, the respective R⁷s, R¹⁶s, R²¹s, R²⁵s or R³³s may be the same with each other or may be different from each other.

The expression phenyl(Cₐ to C_{b}) alkoxy or a phenyl(Cₐ to C_{b}) alkoxy substituted by (Z)ₚ₁ in the present specification represents a (Cₐ to C_{b}) alkoxy group having the above-mentioned meaning optionally substituted by a phenyl group or phenyl group substituted by (Z)ₚ₁, and as the (Cₐ to C_{b}) alkoxy group, there may be mentioned, for example, —CH₂O— group, —CH(CH₃)O— group, —C(CH₃)₂O— group, —CH₂CH₂O— group, —CH(CH₃)CH₂O— group, —C(CH₃)₂CH₂O— group, etc., and each may be selected from the range of the carbon numbers as designated, respectively.

The expression Cₐ to C_{b} haloalkoxy(C_{d} to Cₑ) haloalkoxy in the present specification represents a haloalkoxy group having the above-mentioned meaning with d to e carbon atoms optionally substituted by a Cₐ to C_{b} haloalkoxy group having the above-mentioned meaning, and there may be mentioned, for example, 1,1,2-trifluoro-2-trifluoromethoxyethoxy group, 1,1,2-trifluoro-2-heptafluoropropyloxyethoxy group, etc., and each may be selected from the range of the carbon numbers as designated, respectively.

The expression phenyl(Cₐ to C_{b})alkylcarbonyl or a phenyl(Cₐ to C_{b})alkylcarbonyl substituted by (Z)ₚ₁ in the present specification represents a (Cₐ to C_{b})alkylcarbonyl group having the above-mentioned meaning and optionally substituted by a phenyl group or a phenyl group substituted by (Zₚ₁, and as the (Cₐ to C_{b})alkylcarbonyl group, there may be mentioned, for example, —CH₂C(O)— group, —CH(CH₃)C(O)— group, —C(CH₃)₂C(O)— group, —CH₂CH₂C(O)— group, —CH(CH₃)CH₂C(O)— group, —C(CH₃)₂CH₂C(O)— group, —CH₂CH(CH₃)C(O)— group, —CH₂C(CH₃)₂C(O)— group, —CH₂CH₂CH₂C(O)— group, etc., and each may be selected from the range of the carbon numbers as designated, respectively.

The expression a phenyl(Cₐ to C_{b}) alkoxycarbonyl or a phenyl(Cₐ to C_{b}) alkoxycarbonyl substituted by (Z)ₚ₁ in the present specification represents a (Cₐ to C_{b}) alkoxycarbonyl group having the above-mentioned meaning and optionally substituted by a phenyl group or a phenyl group substituted by (Z)ₚ₁, and as the (Cₐ to C_{b}) alkoxycarbonyl group, there may be mentioned, for example, —CH₂O—C(O)— group, —CH(CH₃)O—C(O)— group, —C(CH₃)₂O—C(O)— group, —CH₂CH₂O—C(O)— group, —CH(CH₃)CH₂O—C(O)— group, —C(CH₃)₂CH₂O—C(O)— group, etc., and each may be selected from the range of the carbon numbers as designated, respectively.

In the present specification, as specific examples of the expressions

“R² is combined with R¹ to form a C₂ to C₆ alkylene chain whereby it may form a 3 to 7-membered ring with the nitrogen atom to which they are bonded, and at this time, the alkylene chain may contain one oxygen atom, sulfur atom or nitrogen atom,”,

“R¹⁷ and R¹⁸ are combined in combination to form a C₄ to C₇ alkylene chain whereby it may form a 5 to 8-membered ring with the nitrogen atom to which they are bonded, and at this time, the alkylene chain may contain one oxygen atom or sulfur atom,”,

“R²⁶ and R²⁷ are combined in combination to form a C₂ to C₅ alkylene chain whereby it may form a 3 to 6-membered ring with the nitrogen atom to which they are bonded, and at this time, the alkylene chain may contain one oxygen atom or sulfur atom,”,

“R²⁹ and R³⁰ are combined in combination to form a C₂ to C₅ alkylene chain whereby it may form a 3 to 6-membered ring with the nitrogen atom to which they are bonded, and at this time, the alkylene chain may contain one oxygen atom, sulfur atom or nitrogen atom,”,

and

“R³⁴ and R³⁵ are combined in combination to form a C₂ to C₅ alkylene chain whereby it may form a 3 to 6-membered ring with the nitrogen atom to which they are bonded, and at this time, the alkylene chain may contain one oxygen atom or sulfur atom,” there may be mentioned, for example, azilidine, azetidine, pyrrolidine, oxazolidine, thiazolidine, imidazolidine, piperidine, morpholine, thiomorpholine, piperazine, homopiperidine, heptamethyleneimine, etc., and each may be selected from the range of the carbon numbers as designated, respectively.

In the present specification, as specific examples of the expression

“R⁹ and R¹⁰ are combined in combination to form a C₂ to C₆ alkylene chain whereby it may form a 3 to 7-membered ring with the nitrogen atom to which they are bonded, and at this time, the alkylene chain may contain one oxygen atom, sulfur atom or nitrogen atom,”

there may be mentioned, for example, azilidine, azetidine, azetidin-2-one, pyrrolidine, pyrrolidin-2-one, oxazolidine, oxazolidin-2-one, thiazolidine, thiazolidin-2-one, imidazolidine, imidazolidin-2-one, piperidine, piperidin-2-one, morpholine, tetrahydro-1,3-oxazin-2-one, thiomorpholine, tetrahydro-1,3-thiazin-2-one, piperazine, tetrahydropyrimidin-2-one, homopiperidine, homopiperidin-2-one, etc., and each may be selected from the range of the carbon numbers as designated, respectively.

In the present specification, as specific examples of the expression

“R¹¹ is combined with R⁹ to form a C₂ to C₄ alkylene chain whereby it may form a 5 to 7-membered ring with the atoms to which they are bonded, and at this time, the alkylene chain may contain one oxygen atom, sulfur atom or nitrogen atom,”

there may be mentioned, for example, isoxazoline, 1,4,2-dioxazoline, 1,4,2-oxathiazoline, 1,2,4-oxadiazoline, dihydro-1,2-oxadine, dihydro-1,4,2-dioxadine, dihydro-1,4,2-oxathizine, dihydro-4H-1,2,4-oxadiazine, tetrahydro-1,2-oxazepine, etc., and each may be selected from the range of the carbon numbers as designated, respectively.

In the compounds included in the present invention, as the substituent represented by W¹ or W², there may be mentioned, for example, oxygen atom or sulfur atom, and of these, the oxygen atom is preferred.

In the compounds included in the present invention, as a scope of the substituent represented by X, there may be mentioned, for example, the following respective groups. At this time, in the respective cases mentioned below, when m is an integer of 2 or more, the respective Xs may be the same with each other or different from each other.

That is, X-I: a halogen atom.

X-II: cyano and nitro.

X-III: a C₁ to C₆ alkyl and a C₁ to C₆ haloalkyl.

X-IV: a C₁ to C₆ alkoxy, a C₁ to C₆ haloalkoxy and a C₁ to C₆ alkylsulfonyloxy.

X-V: a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl and a C₁ to C₆ haloalkylsulfonyl.

X-VI: m is an integer of 2 or more, and, two Xs are adjacent to each other, and further adjacent two Xs form —OCH₂O— or —OCH₂CH₂O—, whereby the form a 5-membered ring or 6-membered ring with carbon atoms to which they are bonded to. At this time, a hydrogen atom bonded to the respective carbon atoms which form the ring may be optionally substituted by a halogen atom, a C₁ to C₄ alkyl group or a C₁ to C₄ haloalkyl group.

In the compounds included in the present invention, as m which represents a number of the substituent represented by X, an integer of 0 to 4 is mentioned, and of these, m is preferably 0, 1 and 2.

In the compounds included in the present invention, as the scope of the substituent represented by Y, there may be mentioned, for example, the following respective groups. At this time, in the respective cases mentioned below, when n is an integer of 2 or more, the respective Ys may be the same with each other or different from each other.

That is, Y-I: a halogen atom.

Y-II: a C₁ to C₆ alkyl.

Y-III: a C₁ to C₆ haloalkyl, a hydroxy(C₁ to C₆) alkyl and a C₁ to C₄ alkoxy(C₁ to C₄)alkyl.

Y-IV: a C₁ to C₆ alkoxy and a C₁ to C₆ haloalkoxy.

Y-V: a C₁ to C₆ alkylthio and a C₁ to C₆ haloalkylthio.

Y-VI: a C₁ to C₆ alkylamino and a di(C₁ to C₆ alkyl)amino.

In the compounds included in the present invention, as n which represents a number of the substituent represented by Y, an integer of 0 to 4 may be mentioned, and of these, n is preferably 0, 1 and 2.

In the compounds included in the present invention, as the scope of the substituent represented by R¹, there may be mentioned, for example, the following respective groups.

That is, R¹-I: a C₁ to CB alkyl and a C₃ to C₈ cycloalkyl.

R¹-II: a C₁ to C₈ alkyl optionally substituted by —OR²⁶ [here, R²⁶ represents a C₁ to C₆ alkyl, a C₁ to C₆ alkylaminocarbonyl or a di(C₁ to C₆ alkyl)aminocarbonyl.].

R¹-III: a C₁ to C₆ alkyl optionally substituted by —CH═NOH or —CH═NOR³¹ [here, R³¹ represents a C₁ to C₆ alkyl.].

R¹-IV: a C₁ to C₈ alkyl optionally substituted by R¹⁶ [here, R¹⁶ represents —OR²⁶, R²⁶ represents a hydrogen atom, a C₁ to C₆ alkyl, a C₁ to C₆ haloalkyl, a C₁ to C₄ alkoxy(C₁ to C₄)alkyl, a C₁ to C₄ alkylthio(C₁ to C₄)alkyl, a phenyl(C₁ to C₄)alkyl, a phenyl(C₁ to C₄) alkyl substituted by (Z)ₚ₁, a C₁ to C₆ alkylcarbonyl, a C₁ to C₆ haloalkylcarbonyl, a C₃ to C₆ cycloalkylcarbonyl, —C(O)N(R³⁰)R²⁹, a di(C₁ to C₆ alkyl)phosphoryl, a di(C₁ to C₆ alkyl)-thiophosphoryl, a tri(C₁ to C₄ alkyl)silyl, phenyl or a phenyl substituted by (Z)ₚ₁, R²⁹ represents a C₁ to C₆ alkyl, a C₁ to C₄ alkoxy(C₁ to C₄)alkyl, a C₁ to C₄ alkylthio(C₁ to C₄)alkyl, a phenyl(C₁ to C₄)alkyl, a phenyl(C₁ to C₄) alkyl substituted by (Z)ₚ₁, a C₃ to C₆ cycloalkyl, a C₃ to C₆ alkenyl, a C₃ to C₆ alkynyl, phenyl or a phenyl substituted by (Z)ₚ₁, R³⁰ represents a hydrogen atom or a C₁ to C₆ alkyl, or R²⁹ and R³⁰ are combined to form a C₂ to C₅ alkylene chain whereby it may form a 3 to 6-membered ring with the nitrogen atom to which they are bonded, and at this time, the alkylene chain may contain one oxygen atom or sulfur atom.], M-4, M-5, M-14, M-15 and M-16.

R¹-V: a C₁ to C₈ alkyl optionally substituted by R¹⁶ [here, R¹⁶ represents cyano, a C₁ to C₆ alkoxycarbonyl, a C₁ to C₆ alkylaminocarbonyl, a di(C₁ to C₆ alkyl)aminocarbonyl, —C(R³²)═NOH or —C(R³²)═NOR³¹, R³¹ represents a C₁ to C₆ alkyl, a phenyl(C₁ to C₄) alkyl or a phenyl(C₁ to C₄) alkyl substituted by (Z)ₚ₁, R³² represents a hydrogen atom or a C₁ to C₆ alkyl.].

R¹-VI: a C₁ to C₈ alkyl optionally substituted by R¹⁶ [here, R¹⁶ represents a halogen atom, a C₃ to C₆ cycloalkyl, a tri(C₁ to C₆ alkyl)silyl, phenyl, a phenyl substituted by (Z)ₚ₁, L-1, L-2, L-3, L-4, L-45, L-46, L-47 or M.].

R¹-VII: a C₁ to C₆ alkyl, a C₁ to C₄ alkylthio(C₁ to C₄)alkyl, a C₁ to C₄ alkylsulfinyl-(C₁ to C₄) alkyl and a C₁ to C₄ alkylsulfonyl(C₁ to C₄)alkyl.

R¹-VIII: a C₃ to C₈ alkenyl and a C₃ to C₈alkynyl.

R¹-IX: a C₁ to C₄ alkylthio(C₁ to C₄)alkyl, a C₁ to C₄ alkylsulfinyl(C₁ to C₄) alkyl and a C₁ to C₄ alkylsulfonyl(C₁ to C₄)alkyl.

R¹-X: a (C₁ to C₈) alkyl optionally substituted by —N(R²⁷)R²⁶ [here, R²⁶ represents a C₁ to C₆ alkoxycarbonyl, a C₁ to C₆ alkylsulfonyl or a di(C₁ to C₆ alkyl)thiophosphoryl, R²⁷ represents a hydrogen atom or a C₁ to C₆ alkyl.].

R¹-XI: a C₁ to C₈ alkyl optionally substituted by R¹⁶ [here, R¹⁶ represents —S(O)ᵣR²⁸ or —SO₂N(R³⁰)R²⁹, R²⁸ represents a C₁ to C₆ alkyl, a C₁ to C₆ haloalkyl, a hydroxy(C₁ to C₄)alkyl, a C₁ to C₄ alkoxy(C₁ to C₄)alkyl, a C₁ to C₄ alkylthio(C₁ to C₄)alkyl, a C₁ to C₄ alkylcarbonyl(C₁ to C₄)alkyl, a C₁ to C₄ alkoxycarbonyl(C₁ to C₄)alkyl, a C₁ to C₄ alkylaminocarbonyl(C₁ to C₄)alkyl, a di(C₁ to C₄ alkyl)aminocarbonyl(C₁ to C₄)alkyl, a tri(C₁ to C₄ alkyl)silyl (C₁ to C₄)alkyl, a phenyl(C₁ to C₄)alkyl, a phenyl(C₁ to C₄) alkyl substituted by (Z)ₚ₁, a C₃ to C₆ alkenyl, a C₃ to C₆ alkynyl, a C₁ to C₆ alkylthio, phenyl, a phenyl substituted by (Z)ₚ₁, L-21, L-35, L-45 or L-48, r is an integer of 0 to 2, R²⁹ represents a C₁ to C₆ alkyl, R³⁰ represents a hydrogen atom or a C₁ to C₆ alkyl.], M-8, M-9, M-17, M-18 and M-19.

R¹-XII: a C₁ to C₈ alkyl optionally substituted by R¹⁶ [here, R¹⁶ represents —N(R²⁷)R²⁶, R²⁶ represents a C₁ to C₆ alkylcarbonyl, a C₃ to C₆cyclo alkylcarbonyl, a C₁ to C₆ alkoxycarbonyl, a di(C₁ to C₆ alkyl)aminocarbonyl, a C₁ to C₆ alkylsulfonyl, a di(C₁ to C₆ alkyl)aminosulfonyl, phenylsulfonyl, a phenylsulfonyl substituted by (Z)ₚ₁ or a di(C₁ to C₆ alkyl)thiophosphoryl, R²⁷ represents a hydrogen atom or a C₁ to C₆ alkyl.], M-13, M-21 and M-22.

R¹-XIII: a 3 to 7-membered ring formed by R¹ and R² in combination is azilidine, azetidine, pyrrolidine, oxazolidine, thiazolidine, piperidine, morpholine, thiomorpholine and homopiperidine.

R¹-XIV: a C₁ to C₆ alkyl, a C₁ to C₄ alkyl optionally substituted by —OR²⁶ [here, R²⁶ represents a C₁ to C₄ alkyl, a C₁ to C₄ alkylaminocarbonyl or a di(C₁ to C₄ alkyl)aminocarbonyl.], a C₁ to C₄ alkylthio(C₁ to C₄)alkyl, a C₁ to C₄ alkylsulfinyl(C₁ to C₄) alkyl and a C₁ to C₄ alkylsulfonyl(C₁ to C₄)alkyl, a (C₁ to C₄) alkyl optionally substituted by —N(R²⁷)R²⁶ [here, R²⁶ represents a C₁ to C₄ alkoxycarbonyl, a C₁ to C₄ alkylsulfonyl or a di(C₁ to C₄ alkyl)thiophosphoryl, and R²⁷ represents a hydrogen atom or a C₁ to C₄ alkyl.] and a C₁ to C₄ alkyl optionally substituted by —CH═NOH or —CH═NOR³¹ [here, R³¹ represents a C₁ to C₄ alkyl.].

In the compounds included in the present invention, as scopes of the substituents represented by R² and R³, there may be mentioned, for example, the following respective groups.

That is, R²-I or R³-I: a hydrogen atom.

R²-II or R³-II: a hydrogen atom and a C₁ to C₆ alkyl.

R²-III or R³-III: a hydrogen atom, a C₁ to C₄ alkoxy(C₁ to C₄)alkyl, a C₁ to C₄ alkylthio(C₁ to C₄) alkyl and a C₁ to C₄ alkylsulfonyl(C₁ to C₄)alkyl.

R²-IV or R³-IV: a hydrogen atom, a C₃ to C₆ alkenyl and C₃ to C₆alkynyl.

R²-V or R³-V: a hydrogen atom, a C₁ to C₆ alkylthio, a C₁ to C₆ haloalkylthio, phenylthio, a phenylthio substituted by (Z)ₚ₁ and —SN(R¹⁸)R¹⁷ [here, R¹⁷ represents a C₁ to C₆ alkyl, a C₁ to C₆ alkoxycarbonyl(C₁ to C₄) alkyl or a C₁ to C₆ alkoxycarbonyl, R¹⁸ represents a C₁ to C₆ alkyl, or R¹⁷ and R¹⁸ are combined in combination to form a C₄ to C₅ alkylene chain, whereby it may form a 5-membered ring or 6-membered ring with the nitrogen atom to which they are bonded, and at this time, the alkylene chain may contain one oxygen atom or sulfur atom, and may be optionally substituted by methyl group or methoxy group.].

In the compounds included in the present invention, as a scope of the substituent represented by R⁴, there may be mentioned, for example, the following respective groups.

That is, R⁴-I: a C₁ to C₆ alkyl and a C₁ to C₆ haloalkyl.

R⁴-II: a C₁ to C₆ alkyl, a C₁ to C₆ haloalkyl, a C₃ to C₈ cycloalkyl and a C₃ to C₈ halocycloalkyl.

R⁴-III: a (C₁ to C₆) alkyl optionally substituted by R²¹ and a (C₁ to C₆) haloalkyl optionally substituted by R²¹ [here, R²¹ represents cyano, a C₃ to C₆ cycloalkyl, a C₃ to C₆ halocycloalkyl, a C₁ to C₆ alkoxy, a C₁ to C₆ haloalkoxy, phenoxy, a phenoxy substituted by (Z)ₚ₁, a C₁ to C₆ alkylthio, a C₁ to C₆ haloalkylthio, phenylthio, a phenylthio substituted by (Z)ₚ₁, a C₁ to C₆ alkylsulfinyl, a C₁ to C₆ haloalkylsulfinyl, a C₁ to C₆ alkylsulfonyl, a C₁ to C₆ haloalkylsulfonyl, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53.].

R⁴-IV: a C₃ to C₆ haloalkenyl, a C₃ to C₆ haloalkynyl, phenyl and a phenyl substituted by (Z)ₚ₁.

In the compounds included in the present invention, as a scope of the substituent represented by R⁵, there may be mentioned, for example, the following respective groups.

That is, R⁵-I: a (C₁ to C₆) alkyl optionally substituted by R²¹, a (C₁ to C₆) haloalkyl optionally substituted by R²¹, a (C₂ to C₆) alkenyl optionally substituted by R²¹ and a (C₂ to C₆) alkynyl optionally substituted by R²¹ [here, R²¹ represents a C₁ to C₆ alkoxy, a C₁ to C₆ haloalkoxy, phenoxy, a phenoxy substituted by (Z)ₚ₁, phenylthio, a phenylthio substituted by (Z)ₚ₁, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53.].

R⁵-II: a (C₁ to C₆) alkyl optionally substituted by R²¹, a (C₁ to C₆) haloalkyl optionally substituted by R²¹, a (C₂ to C₆) alkenyl optionally substituted by R²¹ and a (C₂ to C₆) alkynyl optionally substituted by R²¹ [here, R²¹ represents cyano, a C₃ to C₆ cycloalkyl, a C₃ to C₆ halocycloalkyl, a C₁ to C₆ alkoxy, a C₁ to C₆ haloalkoxy, phenoxy, a phenoxy substituted by (Z)ₚ₁, a C₁ to C₆ alkylthio, a C₁ to C₆ haloalkylthio, phenylthio, a phenylthio substituted by (Z)ₚ₁, a C₁ to C₆ alkylsulfinyl, a C₁ to C₆ haloalkylsulfinyl, a C₁ to C₆ alkylsulfonyl, a C₁ to C₆ haloalkylsulfonyl, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53.].

R⁵-III: phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 and L-53.

R⁵-IV: a C₃ to C₈ cycloalkyl, a C₃ to C₈ halocycloalkyl, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-25 to L-35, L-37, L-38, L-40, L-43 to L-58, M-4, M-5, M-8, M-9, M-14 to M-18 and M-19.

R⁵-V: cyano, —C(O)OR⁹, —C(O)SR⁹, —C(O)NHR¹⁰, —C(O)N(R¹⁰)R⁹, —C(S)OR⁹, —C(S)SR⁹, —C(S)NHR¹⁰ and —C(S)N(R¹⁰)R⁹ [here, R⁹ represents a C₁ to C₆ alkyl, a C₁ to C₆ haloalkyl, a C₃ to C₆ cycloalkyl(C₁ to C₄)alkyl, a phenyl(C₁ to C₄)alkyl, a phenyl(C₁ to C₄) alkyl substituted by (Z)ₚ₁, a C₃ to C₈ cycloalkyl, a C₃ to C₈ halocycloalkyl, phenyl or a phenyl substituted by (Z)ₚ₁, R¹⁰ represents a hydrogen atom or a C₁ to C₆ alkyl, or R⁹ and R¹⁰ are combined to form a C₄ to C₅ alkylene chain whereby it may form a 5-membered ring or 6-membered ring with the nitrogen atom to which they are bonded, and at this time, the alkylene chain may contain one oxygen atom or sulfur atom.].

R⁵-VI: it forms a C₂ to C₃ alkylene chain with Y existing at the adjavent portion whereby it forms a 5 to 6-membered ring which fuses with a benzene ring, and at this time, the alkylene chain may contain one oxygen atom, sulfur atom or nitrogen atom, and may be optionally substituted by a halogen atom or a C₁ to C₆ haloalkyl group.

In the compounds included in the present invention, as a scope of the substituent represented by R⁶, there may be mentioned, for example, the following respective groups.

That is, R⁶-I: a hydrogen atom.

R⁶-II: a hydrogen atom, a C₁ to C₆ alkyl, a C₁ to C₄ alkoxy(C₁ to C₄)alkyl, a C₁ to C₆ alkylcarbonyl and a tri(C₁ to C₄ alkyl)silyl.

R⁶-III: a hydrogen atom, a C₁ to C₆ alkyl, a C₁ to C₆ haloalkyl, a C₁ to C₄ alkoxy(C₁ to C₄)alkyl, a C₁ to C₄ alkylthio(C₁ to C₄)alkyl, cyano(C₁ to C₆)alkyl, a phenyl(C₁ to C₄)alkyl, a phenyl(C₁ to C₄) alkyl substituted by (Z)ₚ₁, a C₃ to C₆ alkenyl, a C₃ to C₆ haloalkenyl, a phenyl(C₃ to C₆) alkenyl, a phenyl(C₃ to C₆) alkenyl substituted by (Z)ₚ₁, a C₃ to C₆ alkynyl, a C₃ to C₆ haloalkynyl, a phenyl(C₃ to C₆) alkynyl, a phenyl(C₃ to C₆) alkynyl substituted by (Z)ₚ₁, —S(O)₂R⁹, —C(O)R⁹, —C(O)NHR¹⁰, —C(O)N(R¹⁰)R⁹, —C(S)NHR¹⁰, —C(S)N(R¹⁰)R⁹ [here, R⁹ represents a C₁ to C₆ alkyl, a C₁ to C₆ haloalkyl, a C₃ to C₆ cycloalkyl(C₁ to C₄)alkyl, a phenyl(C₁ to C₄)alkyl, a phenyl(C₁ to C₄) alkyl substituted by (Z)ₚ₁, a C₃ to C₈ cycloalkyl, a C₃ to C₈ halocycloalkyl, phenyl or a phenyl substituted by (Z)ₚ₁, R¹⁰ represents a hydrogen atom or a C₁ to C₆ alkyl, or R⁹ and R¹⁰ are combined to form a C₄ to C₅ alkylene chain, whereby it may form a 5-membered ring or 6-membered ring with a nitrogen atom to which they are bonded to, and the alkylene chain at this time may contain one oxygen atom or sulfur atom.], —Si(R¹³)(R¹⁴)R¹² [here, R¹² represents a C₁ to C₆ alkyl, phenyl or a phenyl substituted by (Z)ₚ₁, R¹³ and R¹⁴ each independently represent a C₁ to C₆ alkyl.], —P(O)(OR²²)₂ and —P(S)(OR²²)₂ [here, R²² represents a C₁ to C₆ alkyl.].

The respective groups showing the scopes of the respective substituents in the compounds included in the present invention can be optionally combined with each other and represent the respective scopes of the compounds of the present invention. Examples of the combination of the scopes with regard to R¹, R⁴, R⁵ and R⁶ may be mentioned, for example, the combinations shown in the following Table 1. Provided that the combinations shown in Table 1 are only for exemplary purpose, and the present invention is not limited by these alone.

TABLE 1

| R¹ | R⁴ | R⁵ | R⁶ |
|---|---|---|---|
| R¹-I | R⁴-I | R⁵-I | R⁶-I |
| R¹-I | R⁴-I | R⁵-I | R⁶-II |
| R¹-I | R⁴-I | R⁵-III | R⁶-I |
| R¹-I | R⁴-I | R⁵-III | R⁶-II |
| R¹-I | R⁴-I | R⁵-VI | R⁶-I |
| R¹-I | R⁴-I | R⁵-VI | R⁶-II |
| R¹-II | R⁴-I | R⁵-I | R⁶-I |
| R¹-II | R⁴-I | R⁵-I | R⁶-II |
| R¹-II | R⁴-I | R⁵-III | R⁶-I |
| R¹-II | R⁴-I | R⁵-III | R⁶-II |
| R¹-II | R⁴-I | R⁵-III | R⁶-III |
| R¹-II | R⁴-I | R⁵-IV | R⁶-I |
| R¹-II | R⁴-I | R⁵-IV | R⁶-II |
| R¹-II | R⁴-I | R⁵-V | R⁶-I |
| R¹-II | R⁴-I | R⁵-V | R⁶-II |
| R¹-II | R⁴-I | R⁵-VI | R⁶-I |
| R¹-II | R⁴-I | R⁵-VI | R⁶-II |
| R¹-II | R⁴-II | R⁵-I | R⁶-I |
| R¹-II | R⁴-II | R⁵-I | R⁶-II |
| R¹-II | R⁴-II | R⁵-III | R⁶-I |
| R¹-II | R⁴-II | R⁵-III | R⁶-II |
| R¹-II | R⁴-II | R⁵-III | R⁶-III |
| R¹-II | R⁴-II | R⁵-IV | R⁶-I |
| R¹-II | R⁴-II | R⁵-IV | R⁶-II |
| R¹-II | R⁴-II | R⁵-V | R⁶-I |
| R¹-II | R⁴-II | R⁵-V | R⁶-II |
| R¹-II | R⁴-II | R⁵-VI | R⁶-I |
| R¹-II | R⁴-II | R⁵-VI | R⁶-II |
| R¹-II | R⁴-III | R⁵-III | R⁶-I |
| R¹-II | R⁴-III | R⁵-III | R⁶-II |
| R¹-II | R⁴-IV | R⁵-III | R⁶-I |
| R¹-III | R⁴-I | R⁵-I | R⁶-I |
| R¹-III | R⁴-I | R⁵-I | R⁶-II |
| R¹-III | R⁴-I | R⁵-III | R⁶-I |
| R¹-III | R⁴-I | R⁵-III | R⁶-II |
| R¹-III | R⁴-I | R⁵-III | R⁶-III |
| R¹-III | R⁴-I | R⁵-IV | R⁶-I |
| R¹-III | R⁴-I | R⁵-IV | R⁶-II |
| R¹-III | R⁴-I | R⁵-V | R⁶-I |
| R¹-III | R⁴-I | R⁵-V | R⁶-II |
| R¹-III | R⁴-I | R⁵-VI | R⁶-I |
| R¹-III | R⁴-I | R⁵-VI | R⁶-II |
| R¹-III | R⁴-II | R⁵-I | R⁶-I |
| R¹-III | R⁴-II | R⁵-I | R⁶-II |
| R¹-III | R⁴-II | R⁵-III | R⁶-I |
| R¹-III | R⁴-II | R⁵-III | R⁶-II |
| R¹-III | R⁴-II | R⁵-III | R⁶-III |
| R¹-III | R⁴-II | R⁵-IV | R⁶-I |
| R¹-III | R⁴-II | R⁵-IV | R⁶-II |
| R¹-III | R⁴-II | R⁵-V | R⁶-I |
| R¹-III | R⁴-II | R⁵-V | R⁶-II |
| R¹-III | R⁴-II | R⁵-VI | R⁶-I |
| R¹-III | R⁴-II | R⁵-VI | R⁶-II |
| R¹-III | R⁴-III | R⁵-III | R⁶-I |
| R¹-III | R⁴-III | R⁵-III | R⁶-II |
| R¹-III | R⁴-IV | R⁵-III | R⁶-I |
| R¹-IV | R⁴-I | R⁵-I | R⁶-I |
| R¹-IV | R⁴-I | R⁵-III | R⁶-I |
| R¹-IV | R⁴-I | R⁵-III | R⁶-II |
| R¹-IV | R⁴-I | R⁵-VI | R⁶-I |
| R¹-V | R⁴-I | R⁵-I | R⁶-I |
| R¹-V | R⁴-I | R⁵-III | R⁶-I |
| R¹-V | R⁴-I | R⁵-III | R⁶-II |
| R¹-V | R⁴-I | R⁵-VI | R⁶-I |
| R¹-VI | R⁴-I | R⁵-I | R⁶-I |
| R¹-VI | R⁴-I | R⁵-III | R⁶-I |
| R¹-VI | R⁴-I | R⁵-III | R⁶-II |
| R¹-VI | R⁴-I | R⁵-VI | R⁶-I |
| R¹-VII | R⁴-I | R⁵-I | R⁶-I |
| R¹-VII | R⁴-I | R⁵-I | R⁶-II |
| R¹-VII | R⁴-I | R⁵-I | R⁶-III |
| R¹-VII | R⁴-I | R⁵-II | R⁶-I |
| R¹-VII | R⁴-I | R⁵-II | R⁶-II |
| R¹-VII | R⁴-I | R⁵-III | R⁶-I |
| R¹-VII | R⁴-I | R⁵-III | R⁶-II |
| R¹-VII | R⁴-I | R⁵-III | R⁶-III |
| R¹-VII | R⁴-I | R⁵-IV | R⁶-I |
| R¹-VII | R⁴-I | R⁵-IV | R⁶-II |
| R¹-VII | R⁴-I | R⁵-IV | R⁶-III |
| R¹-VII | R⁴-I | R⁵-V | R⁶-I |
| R¹-VII | R⁴-I | R⁵-V | R⁶-II |
| R¹-VII | R⁴-I | R⁵-VI | R⁶-I |
| R¹-VII | R⁴-I | R⁵-VI | R⁶-II |
| R¹-VII | R⁴-I | R⁵-VI | R⁶-III |
| R¹-VII | R⁴-II | R⁵-I | R⁶-I |
| R¹-VII | R⁴-II | R⁵-I | R⁶-II |
| R¹-VII | R⁴-II | R⁵-II | R⁶-I |
| R¹-VII | R⁴-II | R⁵-III | R⁶-I |
| R¹-VII | R⁴-II | R⁵-III | R⁶-II |
| R¹-VII | R⁴-II | R⁵-IV | R⁶-I |
| R¹-VII | R⁴-II | R⁵-IV | R⁶-II |
| R¹-VII | R⁴-II | R⁵-V | R⁶-I |
| R¹-VII | R⁴-II | R⁵-VI | R⁶-I |
| R¹-VII | R⁴-II | R⁵-VI | R⁶-II |
| R¹-VII | R⁴-III | R⁵-I | R⁶-I |
| R¹-VII | R⁴-III | R⁵-I | R⁶-II |
| R¹-VII | R⁴-III | R⁵-III | R⁶-I |
| R¹-VII | R⁴-III | R⁵-III | R⁶-II |
| R¹-VII | R⁴-III | R⁵-IV | R⁶-I |
| R¹-VII | R⁴-III | R⁵-V | R⁶-I |
| R¹-VII | R⁴-III | R⁵-VI | R⁶-I |
| R¹-VII | R⁴-IV | R⁵-I | R⁶-I |
| R¹-VII | R⁴-IV | R⁵-I | R⁶-II |
| R¹-VII | R⁴-IV | R⁵-III | R⁶-I |
| R¹-VII | R⁴-IV | R⁵-III | R⁶-II |
| R¹-VII | R⁴-IV | R⁵-IV | R⁶-I |
| R¹-VII | R⁴-IV | R⁵-V | R⁶-I |
| R¹-VII | R⁴-IV | R⁵-VI | R⁶-I |
| R¹-VIII | R⁴-I | R⁵-I | R⁶-I |
| R¹-VIII | R⁴-I | R⁵-I | R⁶-II |
| R¹-VIII | R⁴-I | R⁵-III | R⁶-I |
| R¹-VIII | R⁴-I | R⁵-III | R⁶-II |
| R¹-VIII | R⁴-I | R⁵-VI | R⁶-I |
| R¹-VIII | R⁴-I | R⁵-VI | R⁶-II |
| R¹-IX | R⁴-I | R⁵-I | R⁶-I |
| R¹-IX | R⁴-I | R⁵-I | R⁶-II |
| R¹-IX | R⁴-I | R⁵-III | R⁶-I |
| R¹-IX | R⁴-I | R⁵-III | R⁶-II |
| R¹-IX | R⁴-I | R⁵-III | R⁶-III |
| R¹-IX | R⁴-I | R⁵-IV | R⁶-I |
| R¹-IX | R⁴-I | R⁵-IV | R⁶-II |
| R¹-IX | R⁴-I | R⁵-V | R⁶-I |
| R¹-IX | R⁴-I | R⁵-V | R⁶-II |
| R¹-IX | R⁴-I | R⁵-VI | R⁶-I |
| R¹-IX | R⁴-I | R⁵-VI | R⁶-II |
| R¹-IX | R⁴-II | R⁵-I | R⁶-I |
| R¹-IX | R⁴-II | R⁵-I | R⁶-II |
| R¹-IX | R⁴-II | R⁵-III | R⁶-I |
| R¹-IX | R⁴-II | R⁵-III | R⁶-II |
| R¹-IX | R⁴-II | R⁵-III | R⁶-III |
| R¹-IX | R⁴-II | R⁵-IV | R⁶-I |
| R¹-IX | R⁴-II | R⁵-IV | R⁶-II |
| R¹-IX | R⁴-II | R⁵-V | R⁶-I |
| R¹-IX | R⁴-II | R⁵-V | R⁶-II |
| R¹-IX | R⁴-II | R⁵-VI | R⁶-I |
| R¹-IX | R⁴-II | R⁵-VI | R⁶-II |
| R¹-IX | R⁴-III | R⁵-III | R⁶-I |
| R¹-IX | R⁴-III | R⁵-III | R⁶-II |
| R¹-IX | R⁴-IV | R⁵-III | R⁶-I |
| R¹-X | R⁴-I | R⁵-I | R⁶-I |
| R¹-X | R⁴-I | R⁵-I | R⁶-II |
| R¹-X | R⁴-I | R⁵-III | R⁶-I |
| R¹-X | R⁴-I | R⁵-III | R⁶-II |
| R¹-X | R⁴-I | R⁵-III | R⁶-III |
| R¹-X | R⁴-I | R⁵-IV | R⁶-I |
| R¹-X | R⁴-I | R⁵-IV | R⁶-II |
| R¹-X | R⁴-I | R⁵-V | R⁶-I |
| R¹-X | R⁴-I | R⁵-V | R⁶-II |
| R¹-X | R⁴-I | R⁵-VI | R⁶-I |
| R¹-X | R⁴-I | R⁵-VI | R⁶-II |
| R¹-X | R⁴-II | R⁵-I | R⁶-I |
| R¹-X | R⁴-II | R⁵-I | R⁶-II |
| R¹-X | R⁴-II | R⁵-III | R⁶-I |
| R¹-X | R⁴-II | R⁵-III | R⁶-II |
| R¹-X | R⁴-II | R⁵-III | R⁶-III |
| R¹-X | R⁴-II | R⁵-IV | R⁶-I |
| R¹-X | R⁴-II | R⁵-IV | R⁶-II |
| R¹-X | R⁴-II | R⁵-V | R⁶-I |
| R¹-X | R⁴-II | R⁵-V | R⁶-II |
| R¹-X | R⁴-II | R⁵-VI | R⁶-I |
| R¹-X | R⁴-II | R⁵-VI | R⁶-II |
| R¹-X | R⁴-III | R⁵-III | R⁶-I |
| R¹-X | R⁴-III | R⁵-III | R⁶-II |
| R¹-X | R⁴-IV | R⁵-III | R⁶-I |
| R¹-XI | R⁴-I | R⁵-I | R⁶-I |
| R¹-XI | R⁴-I | R⁵-III | R⁶-I |
| R¹-XI | R⁴-I | R⁵-III | R⁶-II |
| R¹-XI | R⁴-I | R⁵-VI | R⁶-I |
| R¹-XII | R⁴-I | R⁵-I | R⁶-I |
| R¹-XII | R⁴-I | R⁵-III | R⁶-I |
| R¹-XII | R⁴-I | R⁵-III | R⁶-II |
| R¹-XII | R⁴-I | R⁵-VI | R⁶-I |
| R¹-XIII | R⁴-I | R⁵-I | R⁶-I |
| R¹-XIII | R⁴-I | R⁵-III | R⁶-I |
| R¹-XIII | R⁴-I | R⁵-III | R⁶-II |
| R¹-XIII | R⁴-I | R⁵-VI | R⁶-I |
| R¹-XIV | R⁴-I | R⁵-I | R⁶-I |
| R¹-XIV | R⁴-I | R⁵-I | R⁶-II |
| R¹-XIV | R⁴-I | R⁵-I | R⁶-III |
| R¹-XIV | R⁴-I | R⁵-II | R⁶-I |
| R¹-XIV | R⁴-I | R⁵-II | R⁶-II |
| R¹-XIV | R⁴-I | R⁵-III | R⁶-I |
| R¹-XIV | R⁴-I | R⁵-III | R⁶-II |
| R¹-XIV | R⁴-I | R⁵-III | R⁶-III |
| R¹-XIV | R⁴-I | R⁵-IV | R⁶-I |
| R¹-XIV | R⁴-I | R⁵-IV | R⁶-II |
| R¹-XIV | R⁴-I | R⁵-IV | R⁶-III |
| R¹-XIV | R⁴-I | R⁵-V | R⁶-I |
| R¹-XIV | R⁴-I | R⁵-V | R⁶-II |
| R¹-XIV | R⁴-I | R⁵-VI | R⁶-I |
| R¹-XIV | R⁴-I | R⁵-VI | R⁶-II |
| R¹-XIV | R⁴-I | R⁵-VI | R⁶-III |
| R¹-XIV | R⁴-II | R⁵-I | R⁶-I |
| R¹-XIV | R⁴-II | R⁵-I | R⁶-II |
| R¹-XIV | R⁴-II | R⁵-II | R⁶-I |
| R¹-XIV | R⁴-II | R⁵-III | R⁶-I |
| R¹-XIV | R⁴-II | R⁵-III | R⁶-II |
| R¹-XIV | R⁴-II | R⁵-IV | R⁶-I |
| R¹-XIV | R⁴-II | R⁵-IV | R⁶-II |
| R¹-XIV | R⁴-II | R⁵-V | R⁶-I |
| R¹-XIV | R⁴-II | R⁵-VI | R⁶-I |
| R¹-XIV | R⁴-II | R⁵-VI | R⁶-II |
| R¹-XIV | R⁴-III | R⁵-I | R⁶-I |
| R¹-XIV | R⁴-III | R⁵-I | R⁶-II |
| R¹-XIV | R⁴-III | R⁵-III | R⁶-I |
| R¹-XIV | R⁴-III | R⁵-III | R⁶-II |
| R¹-XIV | R⁴-III | R⁵-IV | R⁶-I |
| R¹-XIV | R⁴-III | R⁵-V | R⁶-I |
| R¹-XIV | R⁴-III | R⁵-VI | R⁶-I |
| R¹-XIV | R⁴-IV | R⁵-I | R⁶-I |
| R¹-XIV | R⁴-IV | R⁵-I | R⁶-II |
| R¹-XIV | R⁴-IV | R⁵-III | R⁶-I |
| R¹-XIV | R⁴-IV | R⁵-III | R⁶-II |
| R¹-XIV | R⁴-IV | R⁵-IV | R⁶-I |
| R¹-XIV | R⁴-IV | R⁵-V | R⁶-I |
| R¹-XIV | R⁴-IV | R⁵-VI | R⁶-I |

The compounds of the present invention can be prepared, for example, by the following methods. 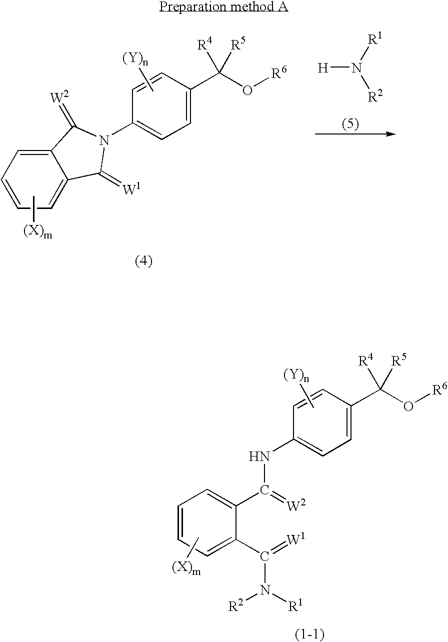
The compound represented by Formula (4) [wherein W¹, W², X, Y, R⁴, R⁵, R⁶, m and n have the same meanings as defined above.] and the compound represented by Formula (5) [wherein R¹ and R² have the same meanings as defined above.] are reacted in a solvent which is inactive to the reaction or in the absence of a solvent, and in the presence of a catalyst if necessary, to obtain the compound of the present invention represented by Formula (1-1) [wherein W¹, W², X, Y, R¹, R², R⁴, R⁵, R⁶, m and n have the same meanings as defined above.] wherein R³ in the formula (1) is a hydrogen atom.

As amounts of the reaction substrates, 1 to 50 equivalents of the compound represented by Formula (5) can be used based on 1 equivalent of the compound represented by Formula (4).

When a solvent is used, the solvent to be used is not limited so long as it does not interfere the progress of the reaction, and there may be mentioned, for example, an aromatic hydrocarbon such as benzene, toluene, xylene, etc., an aliphatic hydrocarbon such as hexane, heptane, etc., an alicyclic hydrocarbon such as cyclohexane, etc., an aromatic halogenated hydrocarbon such as chlorobenzene, dichlorobenzene, etc., an aliphatic halogenated hydrocarbon such as dichloromethane, chloroform, carbon tetrachloride, 1,2-dichloroethane, 1,1,1-trichloroethane, trichloroethylene, tetrachloroethylene, etc., an ether such as diethyl ether, 1,2-dimethoxyethane, tetrahydrofuran, 1,4-dioxane, etc., an ester such as ethyl acetate, ethyl propionate, etc., an amide such as dimethylformamide, dimethylacetamide, N-methyl-2-pyrrolidone, etc., a carboxylic acid such as formic acid, acetic acid, propionic acid, etc., an amine such as triethylamine, tributyl amine, N,N-dimethylaniline, etc., a pyridine such as pyridine, picoline, etc., an alcohol such as methanol, ethanol, ethylene glycol, etc., acetonitrile, dimethylsulfoxide, sulfolane, 1,3-dimethyl-2-imidazolidinone and water, etc. These solvents may be used alone, or may be used in combination of two or more kinds.

When a catalyst is used, the catalyst for the reaction may be mentioned, for example, mineral acids such as hydrochloric acid, sulfuric acid, nitric acid, etc., organic acids such as formic acid, acetic acid, propionic acid, trifluoroacetic acid, methanesulfonic acid, benzenesulfonic acid, p-toluenesulfonic acid, etc., acid addition salts of an amine such as triethylamine hydrochloride, pyridine hydrochloride, etc., Lewis acids such as zinc chloride, zinc iodide, titanium tetrachloride, cerium chloride, ytterbium trifrate, boron trifluoride-ether complex, etc., in an amount of 0.001 to 1 equivalent based on the compound represented by Formula (4).

The reaction temperature may be set at an optional temperature from 60° C. to a reflux temperature of the reaction mixture, and the reaction time may vary depending on the concentrations of the reaction substrates, and the reaction temperature, and may be optionally set usually in the range of 5 minutes to 100 hours.

In general, the reaction is preferably carried out by using, for example, 1 to 10 equivalents of the compound represented by Formula (5) based on 1 equivalent of the compound represented by Formula (4), in the absence of a solvent, or using a solvent such as tetrahydrofuran or 1,4-dioxane, etc., in the temperature range of from 50° C. to a reflux temperature of the reaction mixture for 30 minutes to 24 hours.

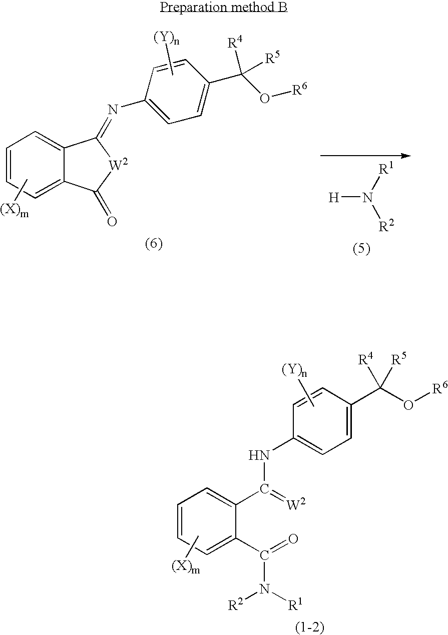

The compound represented by Formula (6) [wherein W², X, Y, R⁴, R⁵, R⁶, m and n have the same meanings as defined above.] and the compound represented by Formula (5) [wherein R¹ and R² have the same meanings as defined above.] are reacted under the similar conditions as in Preparation method A to obtain the compound of the present invention represented by Formula (1-2) [wherein W², X, Y, R¹, R², R⁴, R⁵, R⁶, m and n have the same meanings as defined above.] where W¹ is an oxygen atom and R³ is a hydrogen atom in Formula (1).

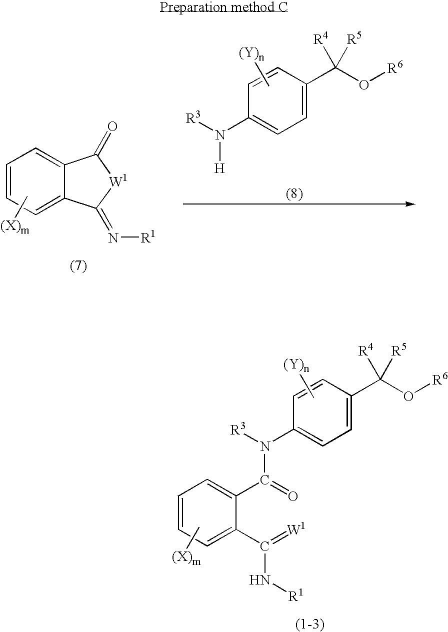

The compound represented by Formula (7) [wherein W¹, X, R¹ and m have the same meanings as defined above.] and the compound represented by Formula (8) [wherein Y, R³, R⁴, R⁵, R⁶ and n have the same meanings as defined above.] are reacted under the similar conditions as in Preparation method A to obtain the compound of the present invention represented by Formula (1-3) [wherein W¹, X, Y, R¹, R³, R⁴, R⁵, R⁶, m and n have the same meanings as defined above.] where W² is an oxygen atom and R² is a hydrogen atom in Formula (1).

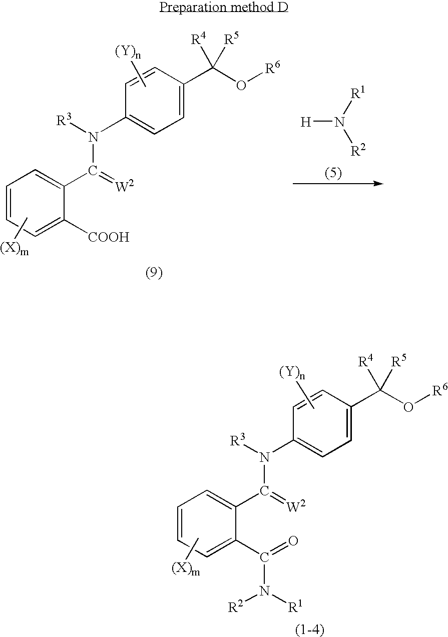

The compound represented by Formula (9) [wherein W², X, Y, R³, R⁴, R⁵, R⁶, m and n have the same meanings as defined above.] and the compound represented by Formula (5) [wherein R¹ and R² have the same meanings as defined above.] are reacted in a solvent which is inactive to the reaction or in the absence of a solvent, and in the presence of a base if necessary, and using a condensing agent to obtain the compound of the present invention represented by Formula (14) [wherein W², X, Y, R¹, R², R³, R⁴, R⁵, R⁶, m and n have the same meanings as defined above.] where W¹ is an oxygen atom in Formula (1).

As amounts of the reaction substrates, 1 to 100 equivalents of the compound represented by Formula (5) can be used based on 1 equivalent of the compound represented by Formula (9).

The condensing agent is not particularly limited so long as it can be used for usual amide synthesis, and there may be mentioned, for example, Mukaiyama reagent (2-chloro-N-methylpyridinium iodide), DCC (1,3-dicyclohexylcarbodiimide), WSC (1-ethyl-3-(3-dimethylaminopropyl)-carbodiimide hydrochloride), a CDI (carbonyldiimidazole), dimethylpropynylsulfonium bromide, propargyltriphenylphosphonium bromide, DEPC (diethyl cyanophosphate), etc., and it can be used in an amount of 1 to 4 equivalents based on the amount of the compound represented by Formula (9).

When a solvent is used, the solvent to be used is not limited so long as it does not interfere the progress of the reaction, and there may be mentioned, for example, aromatic hydrocarbons such as benzene, toluene, xylene, etc., aliphatic hydrocarbons such as hexane, heptane, etc., alicyclic hydrocarbons such as cyclohexane, etc., aromatic halogenated hydrocarbons such as chlorobenzene, dichlorobenzene, etc., aliphatic halogenated hydrocarbons such as dichloromethane, chloroform, carbon tetrachloride, 1,2-dichloroethane, 1,1,1-trichloroethane, trichloroethylene, tetrachloroethylene, etc., ethers such as diethyl ether, 1,2-dimethoxyethane, tetrahydrofuran, 1,4-dioxane, etc., esters such as ethyl acetate, ethyl propionate, etc., amides such as dimethylformamide, dimethylacetamide, N-methyl-2-pyrrolidone, etc., amines such as triethylamine, tributyl amine, N,N-dimethylaniline, etc., pyridines such as pyridine, picoline, etc., acetonitrile and dimethylsulfoxide, etc. These solvents may be used alone, or may be used in combination of two or more kinds

Addition of a base is not necessarily required, and when the base is used, the base to be used may be mentioned, for example, alkali metal hydroxides such as sodium hydroxide, potassium hydroxide, etc., alkali metal carbonates such as sodium carbonate, potassium carbonate, sodium hydrogen carbonate, etc., organic bases such as triethylamine, tributylamine, N,N-dimethylaniline, pyridine, 4-(dimethylamino)pyridine, imidazole, 1,8-diazabicyclo[5,4,0]-7-undecene, etc., and it can be used in an amount of 1 to 4 equivalents based on the amount of the compound represented by Formula (9).

The reaction temperature can be set an optional temperature from 40° C. to the reflux temperature of the reaction mixture, and the reaction time may vary depending on the concentrations of the reaction substrates and the reaction temperature, but it can be optionally set usually within the range of from 5 minutes to 100 hours.

In general, the reaction is preferably carried out by using, for example, 1 to 20 equivalents of the compound represented by Formula (5) and 1 to 4 equivalents of a condensing agent such as WSC (1-ethyl-3-(3-dimethylaminopropyl)-carbodiimide hydrochloride), CDI (carbonyldiimidazole), etc., based on 1 equivalent of the compound represented by Formula (9), and if necessary, in the presence of 1 to 4 equivalents of a base such as potassium carbonate, triethylamine, pyridine, 4-(dimethylamino)pyridine, etc., in the absence of a solvent or in the presence of a solvent such as dichloromethane, chloroform, diethyl ether, tetrahydrofuran, 1,4-dioxane, etc., in the range of 0° C. to a reflux temperature of these solvents, for 10 minutes to 24 hours.

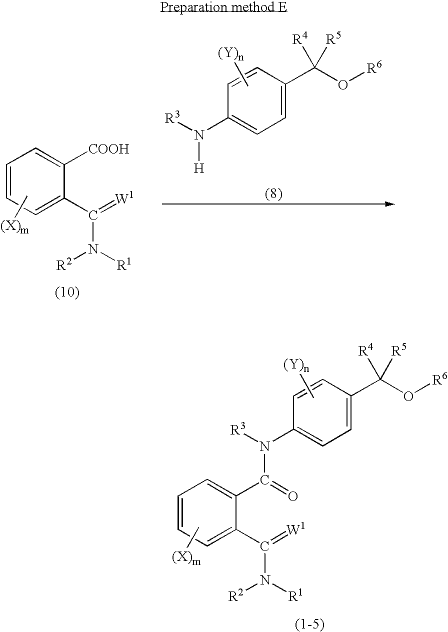

The compound represented by Formula (10) [wherein W¹, X, R¹, R² and m have the same meanings as defined above.] and the compound represented by Formula (8) [wherein Y, R³, R⁴, R⁵, R⁶ and n have the same meanings as defined above.] are reacted under the similar conditions as in Preparation method D to obtain the compound of the present invention represented by Formula (1-5) [wherein W¹, X, Y, R¹, R², R³, R⁴, R⁵, R⁶, m and n have the same meanings as defined above.] where W² is an oxygen atom in Formula (1).

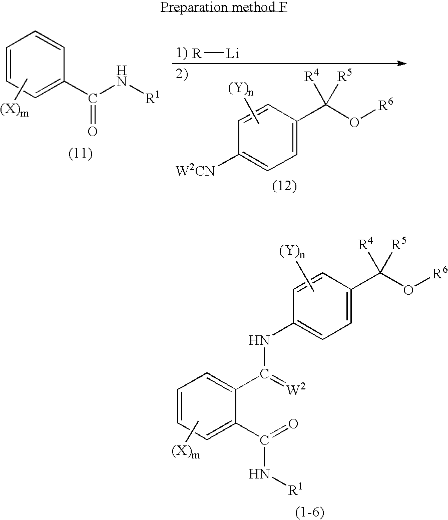

The compound represented by Formula (11) [wherein X, R¹ and m have the same meanings as defined above.] is subjected to selective lithiation according to the conventionally known method described in a literature, for example, the method described in Chemical Reviews [Chem. Rev.] 1990, vol. 90, p. 879, etc., and then, reacting with the compound represented by Formula (12) [wherein W², Y, R⁴, R⁵, R⁶ and n have the same meanings as defined above.] to obtain the compound of the present invention represented by Formula (1-6) [wherein W², X, Y, R¹, R⁴, R⁵, R⁶, m and n have the same meanings as defined above.] where W¹ is an oxygen atom, and R² and R³ are hydrogen atoms in Formula (1). Incidentally, R—Li represents an alkyl lithium reagent such as butyl lithium, etc.

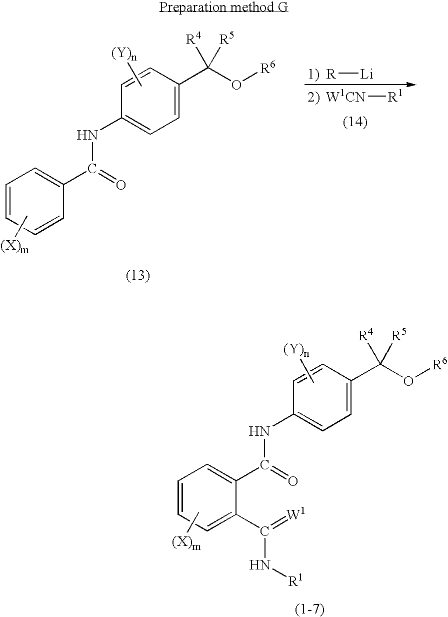

The compound represented by Formula (13) [wherein X, Y, R⁴, R⁵, R⁶, m and n have the same meanings as defined above.] and the compound represented by Formula (14) [wherein W¹ and R¹ have the same meanings as defined above.] are reacted under similar conditions as in Preparation method F to obtain the compound of the present invention represented by Formula (1-7) [wherein W¹, X, Y, R¹, R⁴, R⁵, R⁶, m and n have the same meanings as defined above.] where W² is an oxygen atom, and R² and R³ are hydrogen atoms in Formula (1). Incidentally, R—Li represents an alkyl lithium reagent such as butyl lithium, etc.

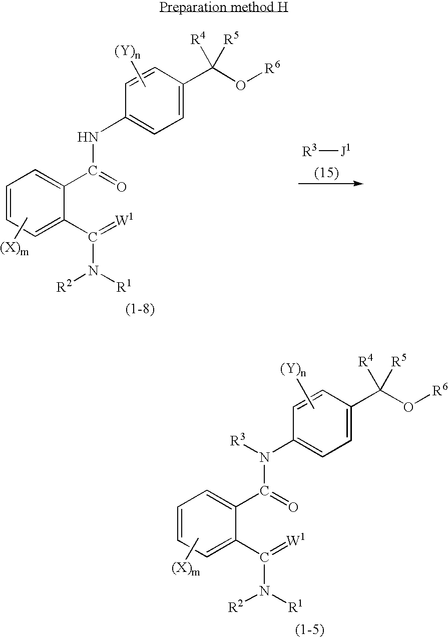

The compound of the present invention represented by Formula (1-8) [wherein W¹, X, Y, R¹, R², R⁴, R⁵, R⁶, m and n have the same meanings as defined above.] where W² is an oxygen atom, and R³ is a hydrogen atom in the compound represented by Formula (1) and the compound represented by Formula (15) [wherein R³ has the same meaning as defined above, and J¹ represents a good eliminating group such as a chlorine atom, a bromine atom, an iodine atom, a C₁ to C₄ alkylcarbonyloxy group (for example, a pivaloyloxy group), a C₁ to C₄ alkylsulfonate group (for example, a methanesulfonyloxy group), a C₁ to C₄ haloalkylsulfonate group (for example, a trifluoromethanesulfonyloxy group), an arylsulfonate group (for example, a benzenesulfonyloxy group, p-toluenesulfonyloxy group) or an azolyl group (for example, an imidazol-1-yl group), etc.] are reacted, if necessary, in the presence of a base, and if necessary, by using a solvent which is inactive to said reaction to obtain the compound of the present invention represented by Formula (1-5) [wherein W¹, X, Y, R¹, R², R³, R⁴, R⁵, R⁶, m and n have the same meanings as defined above.] where W¹ is an oxygen atom in Formula (1).

As amounts of the reaction substrates, 1 to 50 equivalents of the compound represented by Formula (15) can be used based on 1 equivalent of the compound represented by Formula (1-8).

When a solvent is used, the solvent to be used is not limited so long as it does not interfere the progress of the reaction, and there may be mentioned, for example, aromatic hydrocarbons such as benzene, toluene, xylene, etc., aliphatic hydrocarbons such as hexane, heptane, etc., alicyclic hydrocarbons such as cyclohexane, etc., aromatic halogenated hydrocarbons such as chlorobenzene, dichlorobenzene, etc., aliphatic halogenated hydrocarbons such as dichloromethane, chloroform, carbon tetrachloride, 1,2-dichloroethane, 1,1,1-trichloroethane, trichloroethylene, tetrachloroethylene, etc., ethers such as diethyl ether, 1,2-dimethoxyethane, tetrahydrofuran, 1,4-dioxane, etc., esters such as ethyl acetate, ethyl propionate, etc., amides such as dimethylformamide, dimethylacetamide, N-methyl-2-pyrrolidone, etc., amines such as triethylamine, tributyl amine, N,N-dimethylaniline, etc., pyridines such as pyridine, picoline, etc., alcohols such as methanol, ethanol, ethylene glycol, etc., acetonitrile, dimethylsulfoxide, sulfolane, 1,3-dimethyl-2-imidazolidinone and water, etc. These solvents may be used alone, or may be used in combination of two or more kinds

When a base is used, as the base to be used, there may be mentioned, for example, alkali metal hydrides such as sodium hydride, potassium hydrides, etc., alkali metal hydroxides such as sodium hydroxide, potassium hydroxide, etc., alkali metal alkoxides such as sodium ethoxide, potassium tert-butoxide, etc., alkali metal amides such as lithium diisopropylamide, lithium hexamethyldisilazane, sodium amide, etc., organic metal compounds such as tertiary butyl lithium, etc., alkali metal carbonates such as sodium carbonate, potassium carbonate, sodium hydrogen carbonate, etc., organic bases such as triethylamine, tributylamine, N,N-dimethylaniline, pyridine, 4-(dimethylamino)pyridine, imidazole, 1,8-diazabicyclo[5,4,0]-7-undecene, etc., and it can be used in an amount of 1 to 4 equivalents based on the amount of the compound represented by Formula (1-8).

The reaction temperature can be set an optional temperature from −60° C. to the reflux temperature of the reaction mixture, and the reaction time may vary depending on the concentrations of the reaction substrates, and the reaction temperature, but it can be optionally set usually within the range of from 5 minutes to 100 hours.

In general, the reaction is preferable carried out by using, for example, 1 to 10 equivalents of the compound represented by Formula (15) based on 1 equivalent of the compound represented by Formula (1-8), in tetrahydrofuran, 1,4-dioxane, acetonitrile or a polar solvent such as N,N-dimethylformamide, etc., and if necessary, by using 1 to 3 equivalents of a base such as sodium hydride, potassium tert-butoxide, potassium hydroxide, potassium carbonate, triethylamine or pyridine, etc. based on 1 equivalent of the compound represented by Formula (1-8) in a temperature range of 0 to 90° C. for 10 minutes to 24 hours.

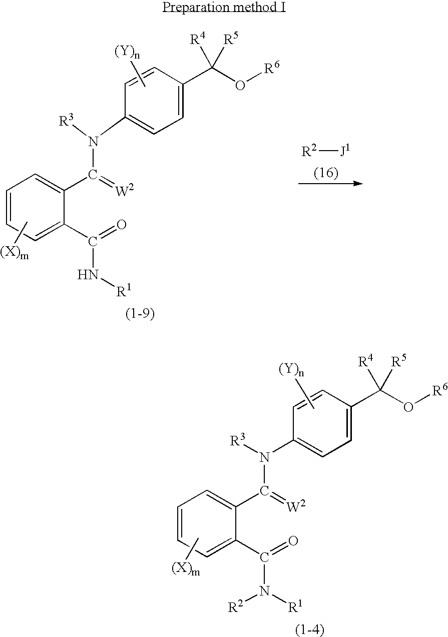

The compound of the present invention represented by Formula (1-9) [wherein W², X, Y, R¹, R³, R⁴, R⁵, R⁶, m and n have the same meanings as defined above.] where W² is an oxygen atom and R² is a hydrogen atom in Formula (1) and the compound represented by Formula (16) [wherein R² and J¹ have the same meanings as defined above.] are reacted under the similar conditions as in Preparation method H to obtain the compound of the present invention represented by Formula (1-4) [wherein W², X, Y, R¹, R², R³, R⁴, R⁵, R⁶, m and n have the same meanings as defined above.] where W¹ is an oxygen atom in Formula (1).

In Preparation method A to Preparation method I, the reaction mixture after completion of the reaction can be subjected to usual post-treatment such as direct concentration, or dissolution in an organic solvent, and after washing with water, subjecting to concentration, or pouring in ice-water, extraction with an organic solvent and then concentration, and the like, to obtain the objective compound of the present invention. Also, when purification is required to be carried out, it can be separated and purified by optional purification methods such as recrystallization, column chromatography, thin layer chromatography, fractionation by liquid chromatography, and the like.

In Preparation method A, the compound represented by Formula (4-1) which is a compound where W¹ and W² are oxygen atoms in the compound represented by Formula (4), which are starting compound for preparing the compound of the present invention, can be synthesized as mentioned below.

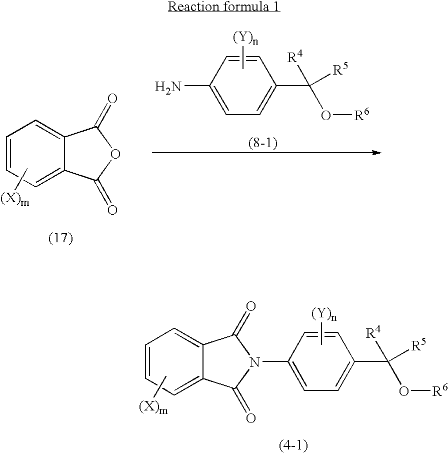

That is, the compound represented by Formula (17) [wherein X and m have the same meanings as defined above.] and the compound represented by Formula (8-1) [wherein Y, R⁴, R⁵, R⁶ and n have the same meanings as defined above.] where R³ is a hydrogen atom in the compound represented by Formula (8) are reacted in accordance with the conventionally known method described in literatures, for example, the methods as described in Berichte der Deutschen Chemischen Gesellschaft [Ber. Dtsch. Chem. Ges.] 1907, vol. 40, p. 3177, Journal of the Chemical Society [J. Chem. Soc.] 1954, p. 2023, Journal of the Chemical Society Perkin Transactions, 1 [J. Chem. Soc. Perkin Trans. 1] 1994, p. 2975, etc., the compound represented by Formula (4-1) [wherein X, Y, R⁴, R⁵, R⁶, m and n have the same meanings as defined above.] where W¹ and W² are oxygen atoms in the compound represented by Formula (4) can be easily synthesized.

Some of the compounds represented by Formula (5) used in Preparation method A, Preparation method B and Preparation method D are conventionally known compounds, and some of which can be obtained as commercially available products. Also, those other than the above can be synthesized according to the methods described in, for example, the methods as described in Chemical and Pharmaceutical Bulletin [Chem. Pharm. Bull.] 1982, vol. 30, p. 1921, Journal of the American Chemical Society [J. Am. Chem. Soc.] 1986, vol. 108, p. 3811, International Patent Publication (WO 01/23350), etc., and the respective synthetic methods of the other primary or secondary alkylamines described in literatures.

In Preparation method B, the compound represented by Formula (6) which is a starting compound for preparing the compound of the present invention can be synthesized as mentioned below.

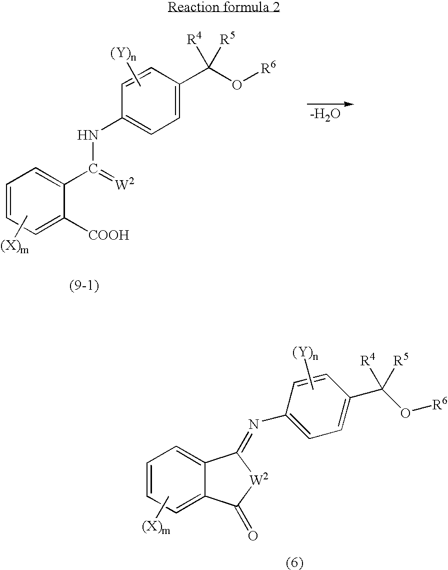

That is, the compound represented by Formula (9-1) [wherein W², X, Y, R⁴, R⁵, R⁶, m and n have the same meanings as defined above.] where R³ is a hydrogen atom in the compound represented by Formula (9) is subjected to cyclization according to the methods such as a synthetic method of isoimide by general dehydration and cyclization described in literatures, for example, the methods described in Journal of the American Chemical Society [J. Am. Chem. Soc.] 1975, vol. 97, p. 5582, Journal of Medicinal Chemistry [J. Med. Chem.] 1967, vol. 10, p. 982, The Journal of Organic Chemistry [J. Org. Chem.] 1963, vol. 28, p. 2018, etc., the compound represented by Formula (6) [wherein W², X, Y, R⁴, R⁵, R⁶, m and n have the same meanings as defined above.] can be easily synthesized.

In Preparation method C, the compound represented by Formula (7) which is a starting compound for preparing the compound of the present invention can be synthesized as mentioned below.

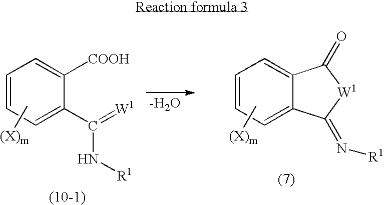

That is, the compound represented by Formula (10-1) [wherein W¹, X, R¹ and m have the same meanings as defined above.] wherein R² is a hydrogen atom in Formula (10) is reacted in the same manner as in Reaction formula 2, the compound represented by Formula (7) [wherein W¹, X, R¹ and m have the same meanings as defined above.] can be easily synthesized.

The compound represented by Formula (8) used in Preparation method C and Preparation method E can be synthesized by, for example, using the method shown by the following Reaction formula 4 to Reaction formula 7 and the like.

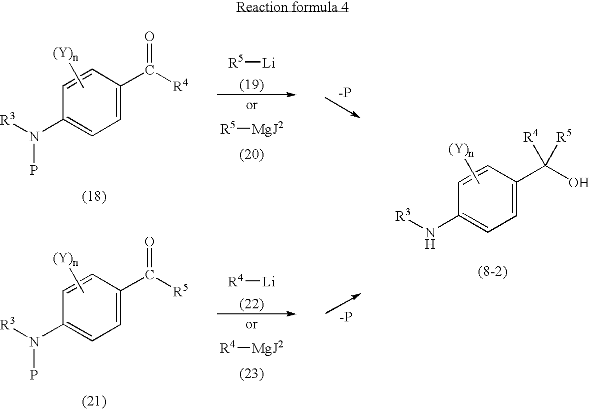

The compound represented by Formula (18) [wherein Y, R³, R⁴ and n have the same meanings as defined above, and P represents a protective group for an amino group generally employed such as acetyl group, pivaloyl group, benzoyl group, tertbutoxycarbonyl group, benzyloxycarbonyl group, etc.] and a lithium reagent of the compound represented by Formula (19) [wherein R⁵ has the same meanings as defined above.] or a Grignard reagent of the compound represented by Formula (20) [wherein R⁵ has the same meaning as mentioned above, and J² represents a halogen atom such as a bromine atom, an iodine atom, etc.] are reacted according to the conventionally knowm method described in literatures, for example, Heterocycles [Heterocycles] 1994, vol. 39, p. 801, Journal of the American Chemical Society [J. Am. Chem. Soc.] 1988, vol. 110, p. 1862, Tetrahedron [Tetrahedron] 1960, vol. 11, p. 252, Tetrahedron Letters [Tetrahedron Lett.] 1995, vol. 36, p. 9117, etc., and subjecting to deprotection by the method generally employed for the respective protective groups, so that the compound represented by Formula (8-2) [wherein Y, R³, R⁴, R⁵ and n have the same meanings as defined above.] wherein R¹⁶ is a hydrogen atom in Formula (8) can be obtained.

Also, the compound represented by Formula (21) [wherein Y, R³, R⁵, n and P have the same meanings as defined above.] and a lithium reagent of the compound represented by Formula (22) [wherein R⁴ has the same meaning as defined above.] or a Grignard reagent of the compound represented by Formula (23) [wherein R⁴ and J² have the same meanings as defined above.] are reacted under the similar conditions as mentioned above, the compound represented by Formula (8-2) can be obtained.

Some of the lithium reagents represented by Formula (19) and by Formula (22) herein used are known compounds, and part thereof can be obtained as a commercially available product. Also, other compounds than the above can be easily synthesized from the conventionally known halogenated compounds according to the general halogen-metal exchange reaction as described in the literatures, for example, Bulletin of the Chemical Society of Japan [Bull. Chem. Soc. Jpn.] 1990, vol. 63, p. 3719, The Journal of Organic Chemistry [J. Org. Chem.] 1983, vol. 48, p. 1449, etc.

Also, some of the Grignard reagents represented by Formula (20) and by Formula (23) herein used are known compounds, and part thereof can be obtained as a commercially available product. Also, other compounds than the above can be easily synthesized from the conventionally known halogenated compounds according to the general preparation methods of Grignard reagents as described in the literatures, for example, Angewante Chemie International Edition in English [Angew. Chem. Int. Ed. Engl.] 1969, vol. 8, p. 279, The Journal of Organic Chemistry [J. Org. Chem.] 1989, vol. 54, p. 4413, etc. from a conventionally known halogenated compound.

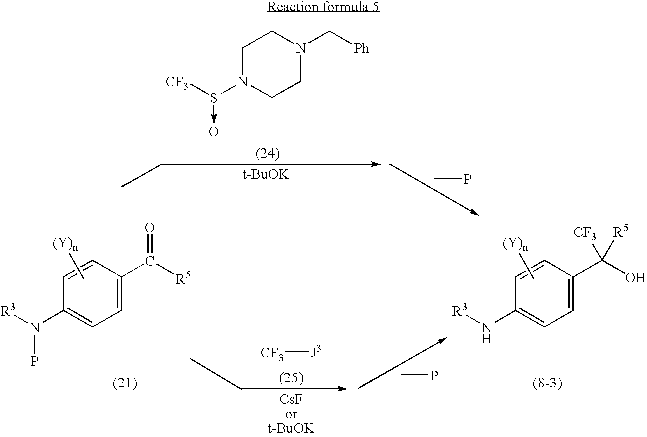

The compound represented by Formula (21) [wherein Y, R³, R⁵, n and P have the same meanings as defined above.] is reacted with the compound represented by Formula (24) by the conventionally known method described in literatures, for example, according to the method described in Synlett [Synlett] 2003, p. 233, or reacted with the conventionally known compound represented by Formula (25) [wherein J³ represents a hydrogen atom, a bromine atom or a trimethylsilyl group.] by the conventionally known method described in literatures, for example, according to the method described in The Journal of Organic Chemistry [J. Org. Chem.] 2000, vol. 65, p. 8848 and 2001, vol. 66, p. 1436, Tetrahedron [Tetrahedron] 1989, vol. 45, p. 1423, etc., and then subjecting to deprotection by the method generally employed for the respective protective groups, so that the compound represented by Formula (8-3) [wherein Y, R³, R⁵ and n have the same meanings as defined above.] wherein R⁴ is a trifluoromethyl group, and R⁶ is a hydrogen atom in Formula (8) can be obtained.

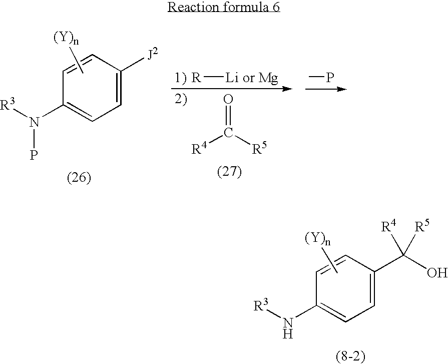

The compound represented by Formula (26) [wherein Y, R³, n, J² and P have the same meanings as defined above.] is subjected according to the general method described in literatures, for example, lithiation and then, reacting with the compound represented by Formula (27) [wherein R⁴ and R⁵ have the same meanings as defined above.] as disclosed in Tetrahedron Letters [Tetrahedron Lett.] 1995, vol. 36, p. 9117, etc., or form a Grignard reagent, and then reacting the resulting material with the compound represented by Formula (27) according to the method as disclosed in Tetrahedron [Tetrahedron] 1960, vol. 11, p. 252, The Journal of Organic Chemistry [J. Org. Chem.] 1988, vol. 53, p. 754. etc., and then subjecting to deprotection by the method generally employed for the respective protective groups, so that the compound represented by Formula (8-2) [wherein Y, R³, R⁴, R⁵ and n have the same meanings as defined above.] wherein R⁶ is a hydrogen atom in Formula (8) can be obtained. Incidentally, R—Li represents an alkyl lithium reagent such as butyl lithium, etc.

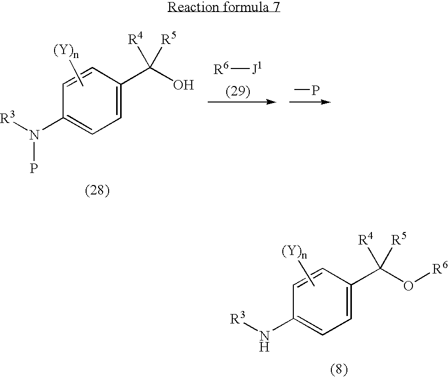

The compound represented by Formula (28) [wherein Y, R³, R⁴, R⁵, n and P have the same meanings as defined above.] which is used as an intermediate in Reaction formula 4, Reaction formula 5 and Reaction formula 6 and the compound represented by Formula (29) [wherein R⁶ and J¹ have the same meanings as defined above.] are reacted under the same conditions as in Preparation method H, and then subjecting to deprotection by the method generally employed for the respective protective groups, so that the compound represented by Formula (8) [wherein Y, R³, R⁴, R⁵ and n have the same meanings as mentioned above, and R⁶ represents the above-mentioned meaning except for the hydrogen atom.] can be obtained.

In Preparation method D, the compound represented by Formula (9) which is a starting material for preparing the compounds of the present invention can be synthesized by the method as disclosed in, for example, the method represented by the following Reaction formula 8 or Reaction formula 9, etc.

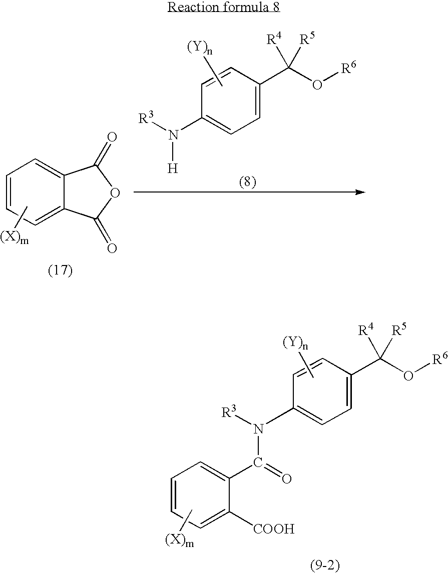

The compound represented by Formula (17) [wherein X and m have the same meanings as defined above.] and the compound represented by Formula (8) [wherein Y, R³, R⁴, R⁵, R⁶ and n have the same meanings as defined above.] are reacted under the similar conditions as in Preparation method A, the compound represented by Formula (9-2) [wherein X, Y, R³, R⁴, R⁵, R⁶, m and n have the same meanings as defined above.] wherein W² is an oxygen atom in the compound represented by Formula (9) can be obtained.

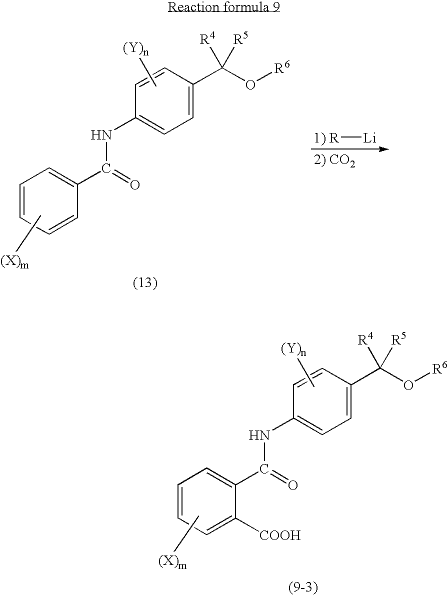

The compound represented by Formula (13) [wherein X, Y, R⁴, R⁵, R⁶, m and n have the same meanings as defined above.] is subjected to selective lithiation according to the conventionally known method described in a literature, for example, the method described in Chemical Reviews [Chem. Rev.] 1990, vol. 90, p. 879, etc., and then, reacting with carbon dioxide to obtain the compound of the present invention represented by Formula (9-3) [wherein X, Y, R⁴, R⁵, R⁶, m and n have the same meanings as defined above.] where W² is an oxygen atom, and R³ is a hydrogen atom in the compound represented by Formula (9). Incidentally, R—Li represents an alkyl lithium reagent such as butyl lithium, etc.

In Preparation method E, the compound represented by Formula (10) which is a starting compound for preparing the compound of the present invention can be synthesized, for example, by the method represented by the following Reaction formula 10 or Reaction formula 11.

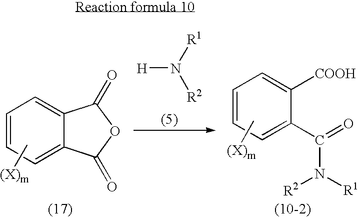

The compound represented by Formula (17) [wherein X and m have the same meanings as defined above.] and the compound represented by Formula (5) [wherein R¹ and R² have the same meanings as defined above.] are reacted under the same conditions as in Preparation method A to prepare the compound represented by Formula (10-2) [wherein X, R¹, R² and m have the same meanings as defined above.] wherein W¹ is an oxygen atom in the compound represented by Formula (10) can be obtained.

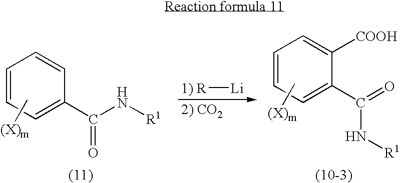

The compound represented by Formula (11) [wherein X, R¹ and m have the same meanings as defined above.] is reacted in the same manner as in Reaction formula 12, the compound represented by Formula (10-3) [wherein X, R¹ and m have the same meanings as defined above.] wherein W¹ is an oxygen atom, and R² is a hydrogen atom in Formula (10) can be obtained. Incidentally, R—Li represents an alkyl lithium reagent such as butyl lithium, etc.

In Preparation method F, some of the compounds represented by Formula (11) which are starting compounds for preparing the compounds of the present invention are known compounds and part thereof can be obtained as commercially available product. Also, those other than the above can be easily synthesized according to the conventionally known methods described in literatures, for example, the methods as described in Bulletin of the Chemical Society of Japan [Bull. Chem. Soc. Jpn.] 1985, vol. 58, p. 3291, The Journal of Organic Chemistry [J. Org. Chem.] 1991, vol. 56, p. 2395, Tetrahedron Letters [Tetrahedron Lett.] 1994, vol. 35, p. 2113, International Patent Publication (WO 98/23581), etc.

The compound represented by Formula (12) to be used in Preparation method F can be synthesized as follows.

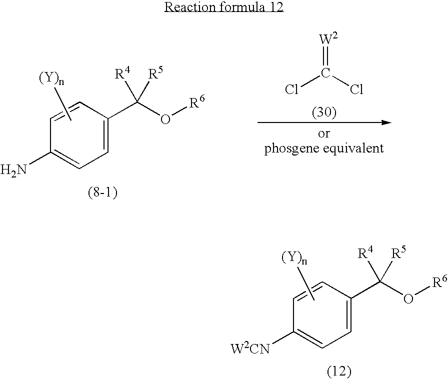

That is, the compound represented by Formula (8-1) [wherein Y, R⁴, R⁵, R⁶ and n have the same meanings as defined above.] wherein R³ is a hydrogen atom in Formula (8) and commercially available phosgene, thiophosgene represented by Formula (30) [wherein W² represents an oxygen atom or sulfur atom.], or equivalents thereof are reacted according to the conventionally known method described in literatures, for example, the method described in Angewante Chemie International Edition in English [Angew. Chem. Int. Ed. Engl.] 1987, vol. 26, p. 894 and 1995, vol. 34, p. 2497, The Journal of Organic Chemistry [J. Org. Chem.] 1976, vol. 41, p. 2070, Synthesis [Synthesis] 1988, p. 990, Tetrahedron Letters [Tetrahedron Lett.] 1997, vol. 38, p. 919, etc., the compound represented by Formula (12) [wherein W², Y, R⁴, R⁵, R⁶ and n have the same meanings as defined above.] can be easily synthesized.

In Preparation method G, the compound represented by Formula (13) which is a starting compound for preparing the compound of the present invention can be synthesized as follows.

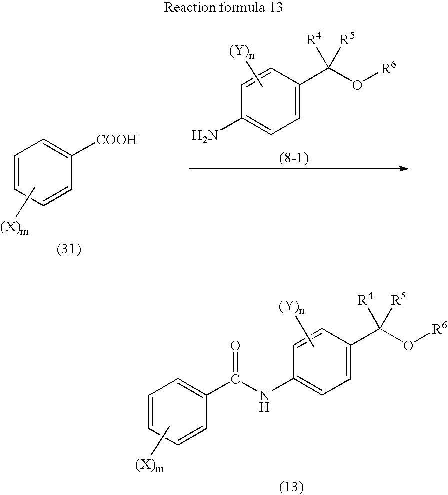

That is, the compound represented by Formula (31) [wherein X and m have the same meanings as defined above.] and the compound represented by Formula (8-1) [wherein Y, R⁴, R⁵, R⁶ and n have the same meanings as defined above.] wherein R³ is a hydrogen atom in the compound represented by Formula (8) are reacted under the same conditions as in Preparation method D, or the compound represented by Formula (31) is converted into a corresponding carboxylic acid chloride using the conventionally knowm methods (for example, a chlorinating agent such as thionyl chloride, phosphorus pentachloride or oxalyl chloride, etc.), and then, reacting with the compound represented by Formula (8-1), whereby the compound represented by Formula (13) [wherein X, Y, R⁴, R⁵, R⁶, m and n have the same meanings as defined above.] can be easily synthesized.

The compound represented by Formula (31) herein used is a conventionally known compound, and some of which can be obtained as a commercially available product.

Some of the compounds represented by Formula (14) used in Preparation method G are conventionally known compounds, and some of which can be obtained as commercially available products. Also, those other than the above can be easily synthesized according to the methods described in, for example, The Journal of Organic Chemistry [J. Org. Chem.] 1996, vol. 61, p. 3883, p. 3929 and p. 6575, Tetrahedron Letters [Tetrahedron Lett.] 1999, vol. 40, p. 363 and p. 6121, etc.

Some of the compounds represented by Formula (15) used in Preparation method H, some of the compounds represented by Formula (16) used in Preparation method I and some of the compound represented by Formula (29) used in Reaction formula 7 are conventionally known compounds, and some of which can be obtained as commercially available products. Also, those other than the above can be easily synthesized according to the methods described in, for example, Chemistry Letters [Chem. Lett.] 1976, p. 373, Journal of The American Chemical Society [J. Am. Chem. Soc.] 1964, vol. 86, p. 4383, The Journal of Organic Chemistry [J. Org. Chem.] 1976, vol. 41, p. 4028 and 1978, vol. 43, p. 3244, Organic Synthesis [Org. Synth.] 1988, Collective Volume 6, p. 101, Tetrahedron Letters [Tetrahedron Lett.] 1972, p. 4339, British Patent (GB 2,161,802 B), European Patent (EP 0,051,273 B), etc.

Some of the compounds represented by Formula (17) are conventionally known compounds, and some of which can be obtained as commercially available products. Also, those other than the above can be easily synthesized, for example, as follows.

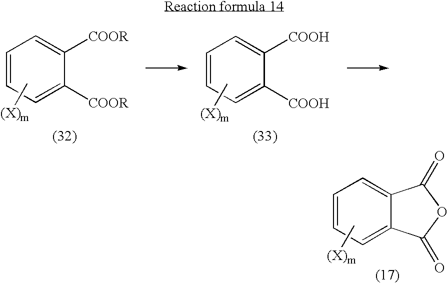

That is, the compound represented by Formula (32) [wherein X and m have the same meanings as defined above, and R represents a lower alkyl group such as methyl group, ethyl group, etc.] is subjected to a general hydrolysis reaction described in literatures, for example, the methods as described in Angewante Chemie [Angew. Chem.] 1951, vol. 63, p. 329, Journal of the American Chemical Society [J. Am. Chem. Soc.] 1929, vol. 51, p. 1865, etc. to form a phthalic acid derivative represented by Formula (33) [wherein X and m have the same meanings as defined above.], and then, subjecting to a general dehydration and cyclization reaction under the conditions described in, for example, the methods as described in The Journal of Organic Chemistry [J. Org. Chem.] 1987, vol. 52, p. 129, etc., so that the compound represented by Formula (17) [wherein X and m have the same meanings as defined above.] can be obtained.

The compound represented by Formula (32) herein used is a conventionally known compound, and some of which can be obtained as a commercially available product.

The compound represented by Formula (18) can be synthesized, for example, as follows.

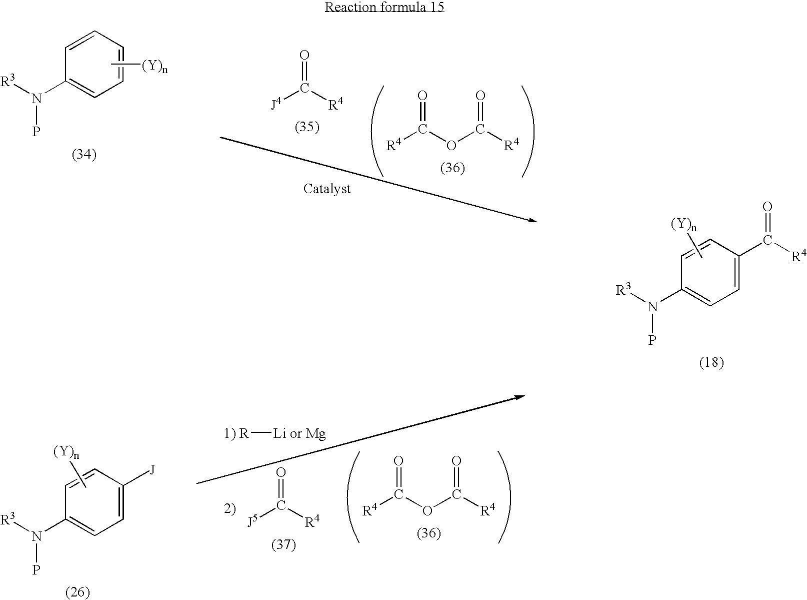

That is, the conventionally known compound represented by Formula (34) [wherein Y, R³, n and P have the same meanings as defined above.] and the conventionally known compound represented by Formula (35) [wherein R⁴ has the same meanings as defined above, J⁴ represents an eliminating group such as a halogen atom, trifluoromethanesulfonyloxy group, 2-pyridyloxy group, etc.] or the conventionally known compound represented by Formula (36) [wherein R⁴ have the same meanings as defined above.] are reacted by a general acylation reaction of the aromatic ring according to the methods described in literatures, for example, the methods as described in Chemistry Letters [Chem. Lett.] 1990, p. 783, The Journal of Organic Chemistry [J. Org. Chem.] 1991, vol. 56, p. 1963, etc., so that the compound represented by Formula (18) [wherein Y, R³, R⁴, n and P have the same meanings as defined above.] can be obtained.

Or else, the compound represented by Formula (26) [wherein Y, R³, n, J² and P have the same meanings as defined above.] is reacted according to the general methods described in literatures, for example, subjecting to lithiation, and then, reacting with the conventionally known compound represented by Formula (37) [wherein R⁴ has the same meanings as defined above, J⁵ represents a halogen atom, a hydroxyl group, a metal salt (e.g., —OLi, —ONa), a C₁ to C₄ alkoxy group (e.g., methoxy group, ethoxy group), a di(C₁ to C₄ alkyl)amino group (e.g., diethylamino group), a C₁ to C₄ alkoxy(C₁ to C₄ alkyl)amino group (e.g., O,N-dimethylhydroxyamino group) or a cyclic amino group (e.g., piperidin-1-yl group, morpholin-4-yl group, 4-methylpiperazin-1-yl group).] or reacting with the conventionally known compound represented by Formula (36) according to the methods as described in Journal of the American Chemical Society [J. Am. Chem. Soc.] 1955, vol. 77, p. 3657, Tetrahedron Letters [Tetrahedron Lett.] 1980, vol. 21, p. 2129 and 1991, vol. 32, p. 2003, U.S. Patent Publication (U.S. Pat. No. 5,514,816), etc., or forming a Grignard reagent, and then, reacting with the compound represented by Formula (37) or the compound represented by Formula (36) according to the methods as described in Heterocycles, 1987, vol. 25, p. 221, Synthetic Communications [Synth. Commun.] 1985, vol. 15, p. 1291 and 1990, vol. 20, p. 1469, German Patent Publication (DE 19727042 A), etc., so that the compound represented by Formula (18) can be obtained.

The compound represented by Formula (21) can be synthesized in the same manner as in the compound represented by Formula (18).

The compound represented by Formula (26) can be synthesized, for example, as follows.

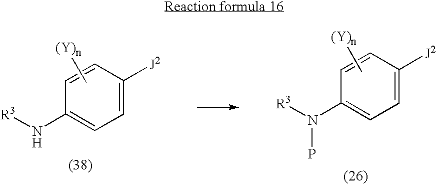

That is, an amino group of the conventionally known substituted aniline represented by Formula (38) [wherein Y, R³, n and J² have the same meanings as defined above.] is protected according to the general method described in literatures, for example, the methods as described in Journal of Medicinal Chemistry [J. Med. Chem.] 1996, vol. 39, p. 673 and 1997, vol. 40, p. 3542, etc., so that the compound represented by Formula (26) [wherein Y, R³, n, J² and P have the same meanings as defined above.] can be obtained.

Some of the compounds represented by Formula (27) are conventionally known compounds, and some of which can be obtained as commercially available products. Also, those other than the above can be synthesized in the same manner as in the compound represented by Formula (18).

In these respective reactions, after completion of the reaction, by carrying out the usual post-treatments, respective preparation intermediates which are starting compounds in Preparation method A to Preparation method I can be obtained.

Also, the respective preparation intermediates prepared by these methods may be used as such in the reaction of the next Step without isolation and purification.

As the compounds included in the present invention, there may be specifically mentioned, for example, those of the compounds shown in Table 2 to Table 5. Provided that the compounds shown in Table 2 to Table 5 are only for the exemplary purpose, and the present invention is not limited only by these.

Incidentally, in the tables, the description Et means an ethyl group, and in the same manner, n-Pr and Pr-n are normal propyl group, i-Pr and Pr-I are isopropyl group, c-Pr and Pr-c are cyclopropyl group, n-Bu and Bu-n are normal butyl group, s-Bu and Bu-s are secondary butyl group, i-Bu and Bu-I are isobutyl group, t-Bu and Bu-t are tertiary butyl group, c-Bu and Bu-c are cyclobutyl group, n-Pen and Pen-n are normal pentyl group, c-Pen and Pen-c are cyclopentyl group, n-Hex and Hex-n are normal hexyl group, c-Hex and Hex-c are cyclohexyl group, Oct are octyl group, Ph is a phenyl group, 1-Naph is 1-naphthyl group, 2-Naph is 2-naphthyl group, respectively,

In the tables, the aromatic heterocycles represented by L-1a to L-55a each represent the following structures, respectively,

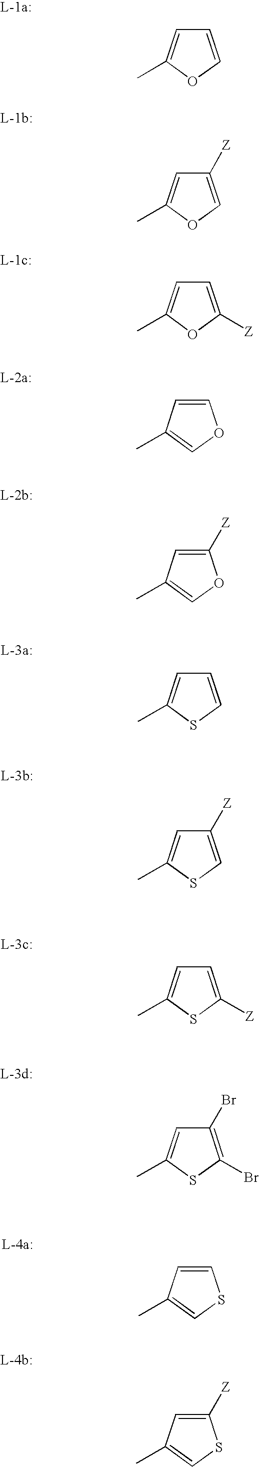

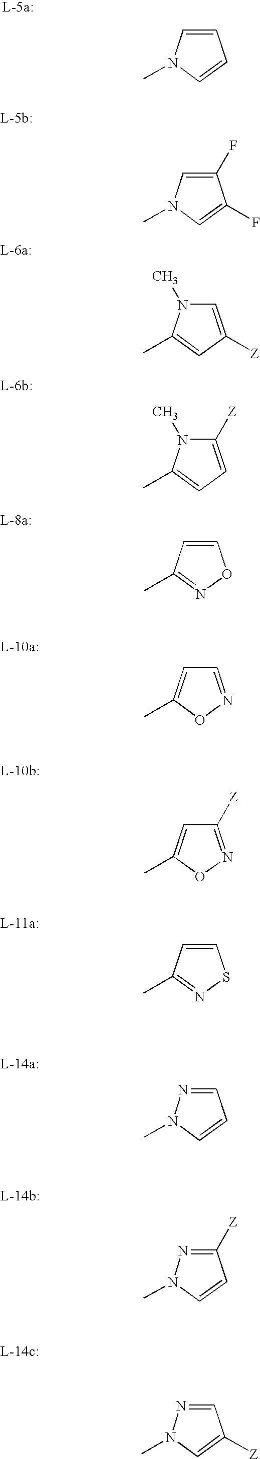

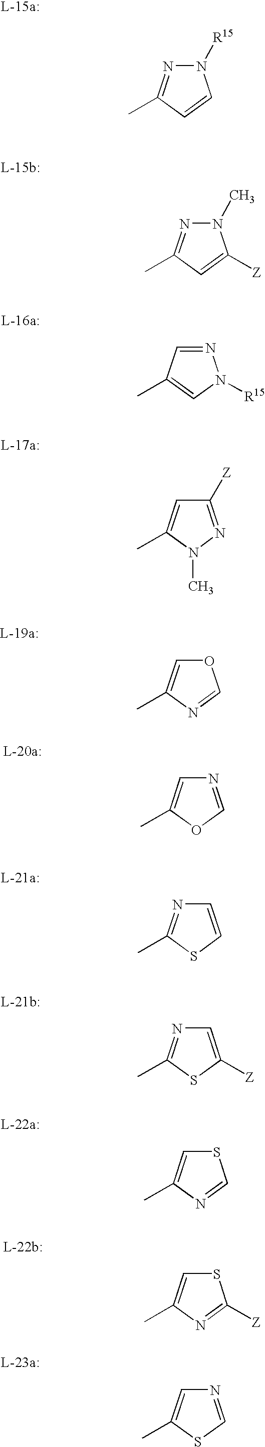

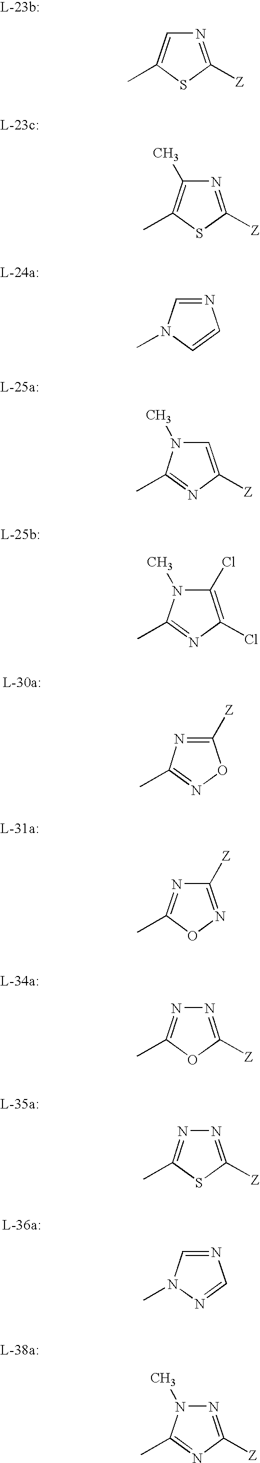

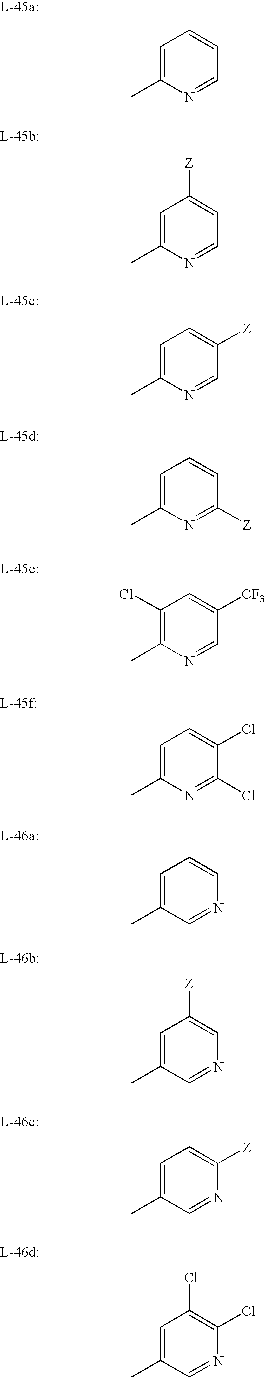

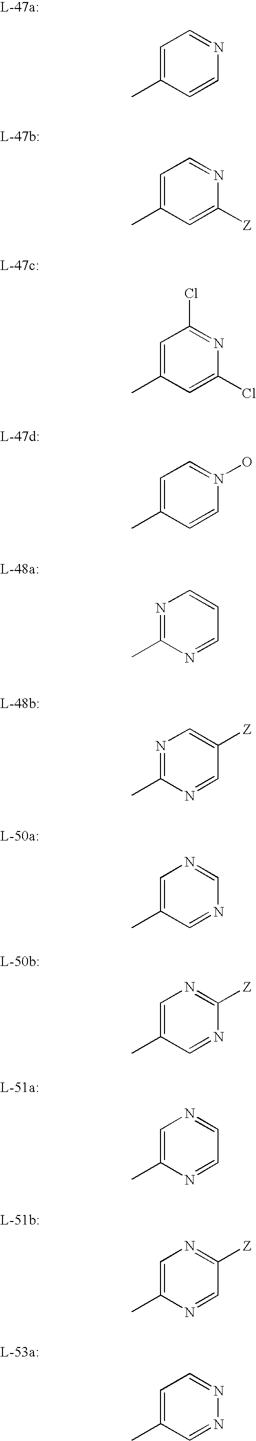

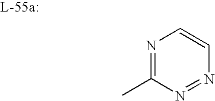

For example, the expression (L-16a)CHF₂ represents a 1-difluoromethylpyrazol-4-yl group, and the expression (L-45c)CF₃ represents a 5-trifluoromethylpyridin-2-yl group.

In the tables, the aliphatic heterocycles represented by M-4a to M-22a each represent the following structures, respectively,

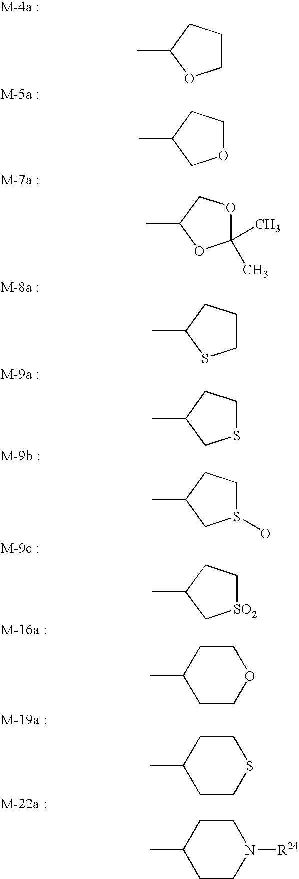

For example, the expression (M-22a)C(O)OCH₃ represents a 1-methoxycarbonylpiperidin-4-yl group.

Moreover, in the tables, T-1 to T-24 each represent the following structures, respectively.

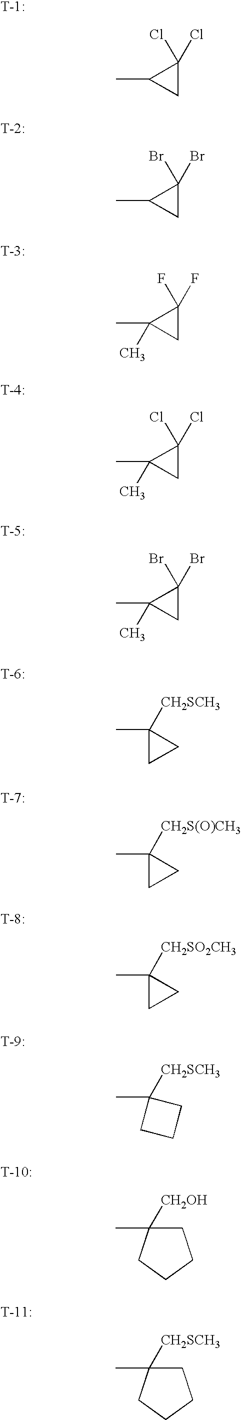

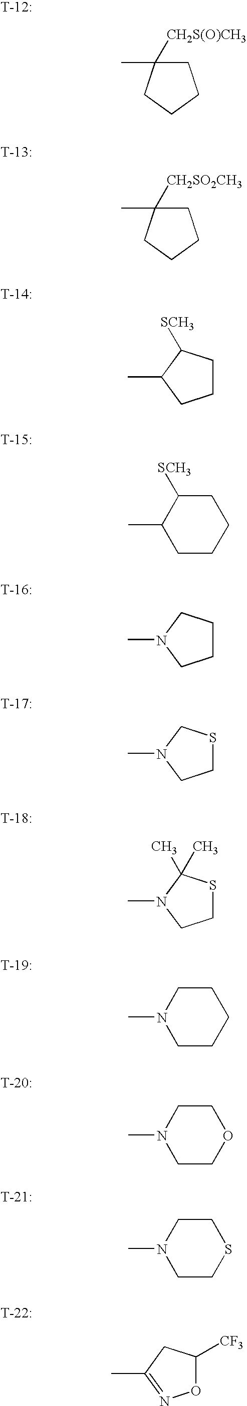

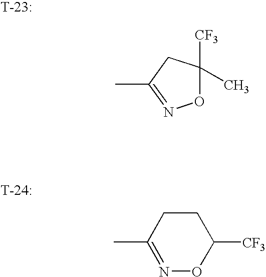

TABLE 2

| 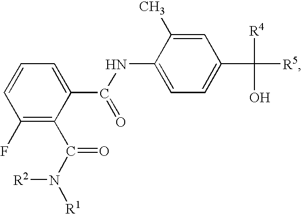 | 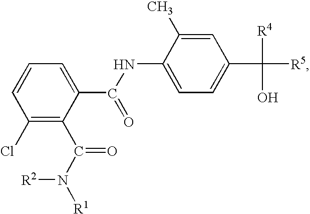 |
|---|---|
| [1] - 1 | [1] - 2 |
| 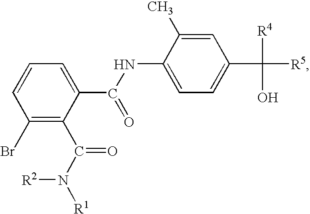 | 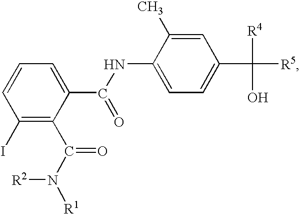 |
| [1] - 3 | [1] - 4 |
| 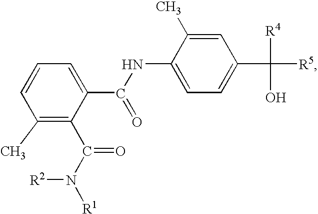 | 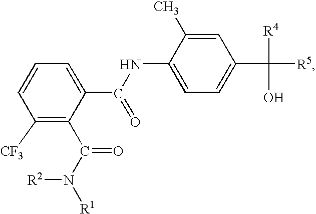 |
| [1] - 5 | [1] - 6 |
| 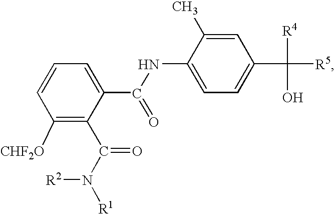 | 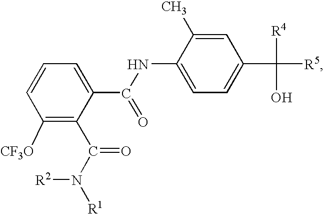 |
| [1] - 7 | [1] - 8 |
| 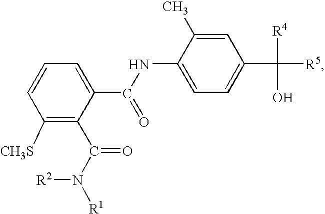 | 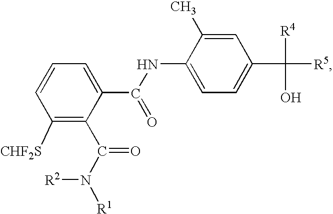 |
| [1] - 9 | [1] - 10 |
| 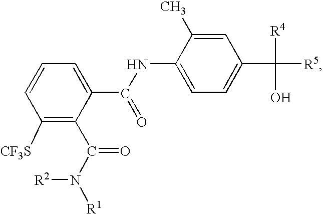 | 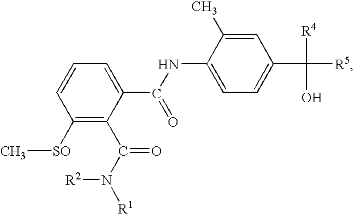 |
| [1] - 11 | [1] - 12 |
| 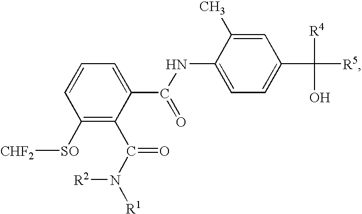 | 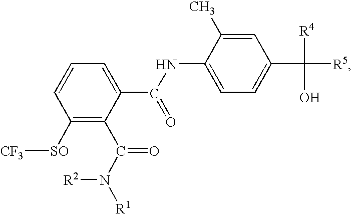 |
| [1] - 13 | [1] - 14 |
| 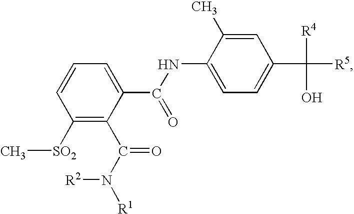 | 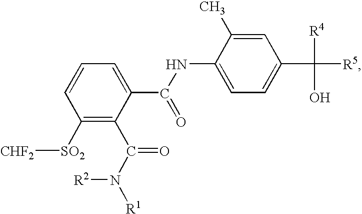 |
| [1] - 15 | [1] - 16 |
| 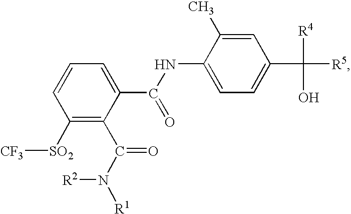 | 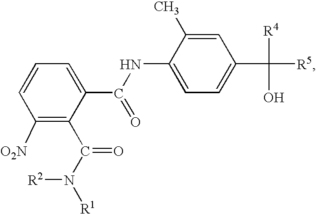 |
| [1] - 17 | [1] - 18 |
| 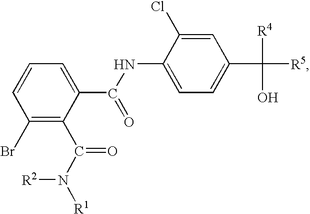 | 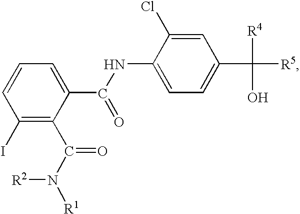 |
| [1] - 19 | [1] - 20 |
| 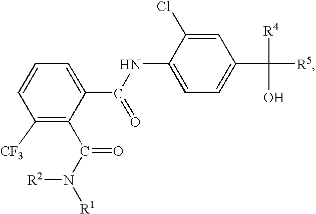 | 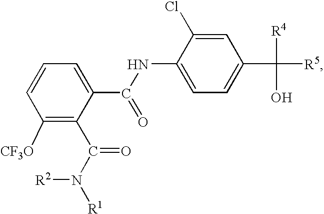 |
| [1] - 21 | [1] - 22 |
| 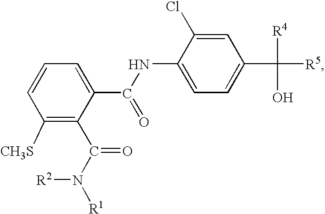 | 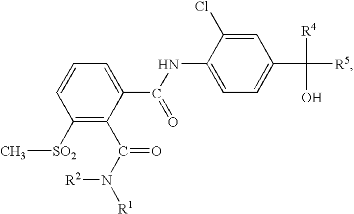 |
| [1] - 23 | [1] - 24 |
| 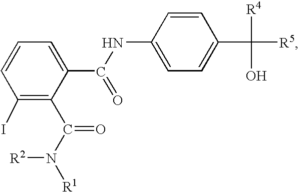 | 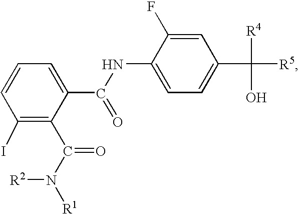 |
| [1] - 25 | [1] - 26 |
| 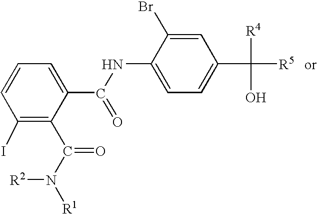 | 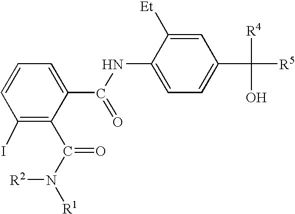 |
| [1] - 27 | [1] - 28 |

| R² | R¹ | R⁴ | R⁵ |
|---|---|---|---|
| H | CH₃ | CF₃ | CF₂O(Ph-4-Cl) |
| H | CH₃ | CF₃ | CH═CH(Ph-4-Cl) |
| H | CH₃ | CF₃ | CH═CH(Ph-4-OCF₃) |
| H | CH₃ | CF₃ | CH═CH(Ph-3 4-Cl₂) |
| H | CH₃ | CF₃ | Ph-4-F |
| H | CH₃ | CF₃ | Ph-4-Cl |
| H | CH₃ | CF₃ | Ph-4-Br |
| H | CH₃ | CF₃ | Ph-4-I |
| H | CH₃ | CF₃ | Ph-4-CF₃ |
| H | CH₃ | CF₃ | Ph-4-OCHF₂ |
| H | CH₃ | CF₃ | Ph-4-OCF₃ |
| H | CH₃ | CF₃ | Ph-4-OCF₂Br |
| H | CH₃ | CF₃ | Ph-4-OCF₂CHF₂ |
| H | CH₃ | CF₃ | Ph-4-OCF₂CHFCl |
| H | CH₃ | CF₃ | Ph-4-OCF₂CHFCF₃ |
| H | CH₃ | CF₃ | Ph-4-OCF₂CHFOCF₃ |
| H | CH₃ | CF₃ | Ph-4-OCF₂CHFOCF₂CF₂CF₃ |
| H | CH₃ | CF₃ | Ph-4-O(L-45e) |
| H | CH₃ | CF₃ | Ph-3,4-Cl₂ |
| H | CH₃ | CF₃ | Ph(-3-OCF₂O-4-) |
| H | CH₃ | CF₃ | Ph(-3-OCF₂CF₂O-4-) |
| H | CH₃ | CF₃ | (L-45c)Cl |
| H | CH₃ | CF₃ | (L-45c)Br |
| H | CH₃ | CF₃ | (L-45c)CF₃ |
| H | CH₃ | CF₃ | L-45e |
| H | CH₃ | CF₃ | L-45f |
| H | CH₃ | CF₃ | (L-46c)Cl |
| H | CH₃ | CF₃ | (L-46c)Br |
| H | CH₃ | CF₃ | (L-46c)CF₃ |
| H | CH₃ | CF₃ | L-46d |
| CH₃ | CH₃ | CF₃ | Ph-4-Br |
| CH₃ | CH₃ | CF₃ | Ph-4-CF₃ |
| CH₃ | CH₃ | CF₃ | Ph-4-OCF₃ |
| CH₃ | CH₃ | CF₃ | (L-45c)CF₃ |
| H | Et | CH₃ | Ph-4-C1 |
| H | Et | CH₃ | Ph-4-CF₃ |
| H | Et | CH₃ | Ph-4-OCF₃ |
| H | Et | CH₃ | (L-45c)Cl |
| H | Et | CH₃ | (L-45c)Br |
| H | Et | CH₃ | (L-45c)CF₃ |
| H | Et | Et | Ph-4-Br |
| H | Et | n-Pr | Ph-4-CF₃ |
| H | Et | i-Pr | Ph-4-OCF₃ |
| H | Et | c-Pr | (L-45c)CF₃ |
| H | Et | CHF₂ | Ph-4-Br |
| H | Et | CF₃ | CF₂O(Ph-4-Cl) |
| H | Et | CF₃ | CH═CH(Ph-4-Cl) |
| H | Et | CF₃ | CH═CH(Ph-4-OCF₃) |
| H | Et | CF₃ | CH═CH(Ph-3,4-Cl₂) |
| H | Et | CF₃ | Ph-4-F |
| H | Et | CF₃ | Ph-4-Cl |
| H | Et | CF₃ | Ph-4-Br |
| H | Et | CF₃ | Ph-4-I |
| H | Et | CF₃ | Ph-4-CF₃ |
| H | Et | CF₃ | Ph-4-OCHF₂ |
| H | Et | CF₃ | Ph-4-OCF₃ |
| H | Et | CF₃ | Ph-4-OCF₂Br |
| H | Et | CF₃ | Ph-4-OCF₂CHF₂ |
| H | Et | CF₃ | Ph-4-OCF₂CHFC1 |
| H | Et | CF₃ | Ph-4-OCF₂CHFCF₃ |
| H | Et | CF₃ | Ph-4-OCF₂CHFOCF₃ |
| H | Et | CF₃ | Ph-4-OCF₂CHFOCF₂CF₂CF₃ |
| H | Et | CF₃ | Ph-4-O(L-45g) |
| H | Et | CF₃ | Ph-3,4-Cl₂ |
| H | Et | CF₃ | Ph(-3-OCF₂O-4-) |
| H | Et | CF₃ | Ph(-3-OCF₂CF₂O-4-) |
| H | Et | CF₃ | (L-45c)Cl |
| H | Et | CF₃ | (L-45c)Br |
| H | Et | CF₃ | (L-45c)CF₃ |
| H | Et | CF₃ | L-45e |
| H | Et | CF₃ | L-45f |
| H | Et | CF₃ | (L-46c)Cl |
| H | Et | CF₃ | (L-46c)Br |
| H | Et | CF₃ | (L-46c)CF₃ |
| H | Et | CF₃ | L-46d |
| H | Et | CF₂Cl | Ph-4-CF₃ |
| H | Et | CF₂Br | Ph-4-OCF₃ |
| H | Et | CF₂CF₃ | (L-45c)CF₃ |
| H | Et | CF₂CF₂CF₃ | Ph-4-Br |
| H | Et | CF₂OCH₃ | Ph-4-CF₃ |
| H | Et | CF₂SCH₃ | Ph-4-OCF₃ |
| Et | Et | CH₃ | Ph-4-Br |
| Et | Et | CH₃ | Ph-4-CF₃ |
| Et | Et | CH₃ | Ph-4-OCF₃ |
| Et | Et | CH₃ | (L-45c)Cl |
| Et | Et | CH₃ | (L-45c)Br |
| Et | Et | CH₃ | (L-45c)CF₃ |
| Et | Et | CF₃ | CF₂O(Ph-4-Cl) |
| Et | Et | CF₃ | CH═CH(Ph-4-Cl) |
| Et | Et | CF₃ | CH═CH(Ph-4-OCF₃) |
| Et | Et | CF₃ | CH═CH(Ph-3,4-Cl₂) |
| Et | Et | CF₃ | Ph-4-F |
| Et | Et | CF₃ | Ph-4-Cl |
| Et | Et | CF₃ | Ph-4-Br |
| Et | Et | CF₃ | Ph-4-I |
| Et | Et | CF₃ | Ph-4-CF₃ |
| Et | Et | CF₃ | Ph-4-OCHF₂ |
| Et | Et | CF₃ | Ph-4-OCF₃ |
| Et | Et | CF₃ | Ph-4-OCF₂Br |
| Et | Et | CF₃ | Ph-4-OCF₂CHF₂ |
| Et | Et | CF₃ | Ph-4-OCF₂CHFCl |
| Et | Et | CF₃ | Ph-4-OCF₂CHFCF₃ |
| Et | Et | CF₃ | Ph-4-OCF₂CHFOCF₃ |
| Et | Et | CF₃ | Ph-4-OCF₂CHFOCF₂CF₂CF₃ |
| Et | Et | CF₃ | Ph-4-O(L-45g) |
| Et | Et | CF₃ | Ph-3, 4-Cl₂ |
| Et | Et | CF₃ | Ph(-3-OCF₂O-4-) |
| Et | Et | CF₃ | Ph(-3-OCF₂CF₂O-4-) |
| Et | Et | CF₃ | (L-45c)Cl |
| Et | Et | CF₃ | (L-45c)Br |
| Et | Et | CF₃ | (L-45c)CF₃ |
| Et | Et | CF₃ | L-45e |
| Et | Et | CF₃ | L-45f |
| Et | Et | CF₃ | (L-46c)Cl |
| Et | Et | CF₃ | (L-46c)Br |
| Et | Et | CF₃ | (L-46c)CF₃ |
| Et | Et | CF₃ | L-46d |
| H | n-Pr | CH₃ | Ph-4-Br |
| H | n-Pr | CH₃ | Ph-4-CF₃ |
| H | n-Pr | CH₃ | Ph-4-OCF₃ |
| H | n-Pr | CH₃ | (L-45c)Cl |
| H | n-Pr | CH₃ | (L-45c)Br |
| H | n-Pr | CH₃ | (L-45c)CF₃ |
| H | n-Pr | Et | (L-45c)CF₃ |
| H | n-Pr | n-Pr | Ph-4-Br |
| H | n-Pr | i-Pr | Ph-4-CF₃ |
| H | n-Pr | c-Pr | Ph-4-OCF₃ |
| H | n-Pr | CHF₂ | (L-45c)CF₃ |
| H | n-Pr | CF₃ | CF₂O(Ph-4-Cl) |
| H | n-Pr | CF₃ | CH═CH(Ph-4-Cl) |
| H | n-Pr | CF₃ | CH═CH(Ph-4-OCF₃) |
| H | n-Pr | CF₃ | CH═CH(Ph-3,4-Cl₂) |
| H | n-Pr | CF₃ | Ph-4-F |
| H | n-Pr | CF₃ | Ph-4-Cl |
| H | n-Pr | CF₃ | Ph-4-Br |
| H | n-Pr | CF₃ | Ph-4-L |
| H | n-Pr | CF₃ | Ph-4-CF₃ |
| H | n-Pr | CF₃ | Ph-4-OCHF₂ |
| H | n-Pr | CF₃ | Ph-4-OCF₃ |
| H | n-Pr | CF₃ | Ph-4-OCF₂Br |
| H | n-Pr | CF₃ | Ph-4-OCF₂CHF₂ |
| H | n-Pr | CF₃ | Ph-4-OCF₂CHFCl |
| H | n-Pr | CF₃ | Ph-4-OCF₂CHFCF₃ |
| H | n-Pr | CF₃ | Ph-4-OCF₂CHFOCF₃ |
| H | n-Pr | CF₃ | Ph-4-OCF₂CHFOCF₂CF₂CF₃ |
| H | n-Pr | CF₃ | Ph-4-O(L-45g) |
| H | n-Pr | CF₃ | Ph-3,4-Cl₂ |
| H | n-Pr | CF₃ | Ph(-3-OCF₂O-4-) |
| H | n-Pr | CF₃ | Ph(-3-OCF₂CF₂O-4-) |
| H | n-Pr | CF₃ | (L-45c)Cl |
| H | n-Pr | CF₃ | (L-45c)Br |
| H | n-Pr | CF₃ | (L-45c)CF₃ |
| H | n-Pr | CF₃ | L-45e |
| H | n-Pr | CF₃ | L-45f |
| H | n-Pr | CF₃ | (L-46c)Cl |
| H | n-Pr | CF₃ | (L-46c)Br |
| H | n-Pr | CF₃ | (L-46c)CF₃ |
| H | n-Pr | CF₃ | L-46d |
| H | n-Pr | CF₂Cl | Ph-4-Br |
| H | n-Pr | CF₂Br | Ph-4-CF₃ |
| H | n-Pr | CF₂CF₃ | Ph-4-OCF₃ |
| H | n-Pr | CF₂CF₂CF₃ | (L-45c)CF₃ |
| H | n-Pr | CF₂OCH₃ | Ph-4-Br |
| H | n-Pr | CF₂SCH₃ | Ph-4-CF₃ |
| CH₃ | n-Pr | CF₃ | Ph-4-Br |
| CH₃ | n-Pr | CF₃ | Ph-4-CF₃ |
| CH₃ | n-Pr | CF₃ | Ph-4-OCF₃ |
| CH₃ | n-Pr | CF₃ | (L-45c)CF₃ |
| Et | n-Pr | CF₃ | Ph-4-OCF₃ |
| n-Pr | n-Pr | CF₃ | (L-45c)CF₃ |
| H | i-Pr | CH₃ | CF₂O(Ph-4-Cl) |
| H | i-Pr | CH₃ | CH═CH(Ph-4-Cl) |
| H | i-Pr | CH₃ | CH═CH(Ph-4-OCF₃) |
| H | i-Pr | CH₃ | CH═CH(Ph-3,4-Cl₂) |
| H | i-Pr | CH₃ | Ph-4-F |
| H | i-Pr | CH₃ | Ph-4-Cl |
| H | i-Pr | CH₃ | Ph-4-Br |
| H | i-Pr | CH₃ | Ph-4-I |
| H | i-Pr | CH₃ | Ph-4-CF₃ |
| H | i-Pr | CH₃ | Ph-4-OCHF₂ |
| H | i-Pr | CH₃ | Ph-4-OCF₃ |
| H | i-Pr | CH₃ | Ph-4-OCF₃Br |
| H | i-Pr | CH₃ | Ph-4-OCF₂CHF₂ |
| H | i-Pr | CH₃ | Ph-4-OCF₂CHFCl |
| H | i-Pr | CH₃ | Ph-4-OCF₂CHFCF₃ |
| H | i-Pr | CH₃ | Ph-4-OCF₂CHFOCF₃ |
| H | i-Pr | CH₃ | Ph-4-OCF₂CHFOCF₂CF₂CF₃ |
| H | i-Pr | CH₃ | Ph-4-O(L-45g) |
| H | i-Pr | CH₃ | Ph-3,4-Cl₂ |
| H | i-Pr | CH₃ | Ph(-3-OCF₂O-4-) |
| H | i-Pr | CH₃ | Ph(-3-OCF₂CF₂O-4-) |
| H | i-Pr | CH₃ | (L-45c)Cl |
| H | i-Pr | CH₃ | (L-45c)Br |
| H | i-Pr | CH₃ | (L-45c)CF₃ |
| H | i-Pr | CH₃ | L-45e |
| H | i-Pr | CH₃ | L-45f |
| H | i-Pr | CH₃ | (L-46c)Cl |
| H | i-Pr | Cl₃ | (L-46c)Br |
| H | i-Pr | CH₃ | (L-46c)CF₃ |
| H | i-Pr | CH₃ | L-46d |
| H | i-Pr | Et | CF₂O(Ph-4-Cl) |
| H | i-Pr | Et | CH═CH(Ph-4-Cl) |
| H | i-Pr | Et | CH═CH(Ph-4-OCF₃) |
| H | i-Pr | Et | CH═CH(Ph-3,4-Cl₂) |
| H | i-Pr | Et | Ph-4-Cl |
| H | i-Pr | Et | Ph-4-Br |
| H | i-Pr | Et | Ph-4-CF₃ |
| H | i-Pr | Et | Ph-4-OCF₃ |
| H | i-Pr | Et | Ph-4-OCF₂Br |
| H | i-Pr | Et | Ph-4-OCF₂CHFCF₃ |
| H | i-Pr | Et | Ph-4-OCF₂CHFOCF₃ |
| H | i-Pr | Et | Ph-4-OCF₂CHFOCF₂CF₂CF₃ |
| H | i-Pr | Et | Ph-4-O(L-45g) |
| H | i-Pr | Et | Ph-3,4-Cl₂ |
| H | i-Pr | Et | Ph(-3-OCF₂O-4-) |
| H | i-Pr | Et | Ph(-3-OCF₂CF₂O-4-) |
| H | i-Pr | Et | (L-45c)Br |
| H | i-Pr | Et | (L-45c)CF₃ |
| H | i-Pr | Et | L-45e |
| H | i-Pr | Et | L-45f |
| H | i-Pr | Et | (L-46c)Br |
| H | i-Pr | Et | (L-46c)CF₃ |
| H | i-Pr | Et | L-46d |
| H | i-Pr | n-Pr | CF₂O(Ph-4-Cl) |
| H | i-Pr | n-Pr | CH═CH(Ph-4-Cl) |
| H | i-Pr | n-Pr | CH═CH(Ph-4-OCF₃) |
| H | i-Pr | n-Pr | CH═CH(Ph-3,4-Cl₂) |
| H | i-Pr | n-Pr | Ph-4-Cl |
| H | i-Pr | n-Pr | Ph-4lBr |
| H | i-Pr | n-Pr | Ph-4-CF₃ |
| H | i-Pr | n-Pr | Ph-4-OCF₃ |
| H | i-Pr | n-Pr | Ph-4-OCF₂Br |
| H | i-Pr | n-Pr | Ph-4-OCF₂CHFCF₃ |
| H | i-Pr | n-Pr | Ph-4-OCF₂CHFOCF₃ |
| H | i-Pr | n-Pr | Ph-4-OCF₂CHFOCF₂CF₂CF₃ |
| H | i-Pr | n-Pr | Ph-4-O(L-45g) |
| H | i-Pr | n-Pr | Ph-3,4-Cl₂ |
| H | i-Pr | n-Pr | Ph(-3-OCF₂O-4-) |
| H | i-Pr | n-Pr | Ph(-3-OCF₂CF₂O-4-) |
| H | i-Pr | n-Pr | (L-45c)Br |
| H | i-Pr | n-Pr | (L-45c)CF₃ |
| H | i-Pr | n-Pr | L-45e |
| H | i-Pr | n-Pr | L-45f |
| H | i-Pr | n-Pr | (L-46c)Br |
| H | i-Pr | n-Pr | (L-46c)CF₃ |
| H | i-Pr | n-Pr | L-46d |
| H | i-Pr | i-Pr | CF₂O(Ph-4-Cl) |
| H | i-Pr | i-Pr | CH═CH(Ph-4-Cl) |
| H | i-Pr | i-Pr | CH═CH(Ph-4-OCF₃) |
| H | i-Pr | i-Pr | CH═CH(Ph-3,4-Cl₂) |
| H | i-Pr | i-Pr | Ph-4-Cl |
| H | i-Pr | i-Pr | Ph-4-Br |
| H | i-Pr | i-Pr | Ph-4-CF₃ |
| H | i-Pr | i-Pr | Ph-4-OCF₃ |
| H | i-Pr | i-Pr | Ph-4-OCF₂Br |
| H | i-Pr | i-Pr | Ph-4-OCF₂CHFCF₃ |
| H | i-Pr | i-Pr | Ph-4-OCF₂CHFOCF₃ |
| H | i-Pr | i-Pr | Ph-4-OCF₂CHFOCF₂CF₂CF₃ |
| H | i-Pr | i-Pr | Ph-4-O(L-45g) |
| H | i-Pr | i-Pr | Ph-3,4-Cl₂ |
| H | i-Pr | i-Pr | Ph(-3-OCF₂O-4-) |
| H | i-Pr | i-Pr | Ph(-3-OCF₂CF₂O-4-) |
| H | i-Pr | i-Pr | (L-45c)Br |
| H | i-Pr | i-Pr | (L-45c)CF₃ |
| H | i-Pr | i-Pr | L-45e |
| H | i-Pr | i-Pr | L-45f |
| H | i-Pr | i-Pr | (L-46c)Br |
| H | i-Pr | i-Pr | (L-46c)CF₃ |
| H | i-Pr | i-Pr | L-46d |
| H | i-Pr | c-Pr | CF₂O(Ph-4-Cl) |
| H | i-Pr | c-Pr | CH═CH(Ph-4-CL) |
| H | i-Pr | c-Pr | CH═CH(Ph-4-OCF₃) |
| H | i-Pr | c-Pr | CH═CH(Ph-3,4-Cl₂) |
| H | i-Pr | c-Pr | Ph-4-Cl |
| H | i-Pr | c-Pr | Ph-4-Br |
| H | i-Pr | c-Pr | Ph-4-CF₃ |
| H | i-Pr | c-Pr | Ph-4-OCF₃ |
| H | i-Pr | c-Pr | Ph-4-OCF₂Br |
| H | i-Pr | c-Pr | Ph-4-OCF₂CHFCF₃ |
| H | i-Pr | c-Pr | Ph-4-OCF₂CHFOCF₃ |
| H | i-Pr | c-Pr | Ph-4-OCF₂CHFOCF₂CF₂CF₃ |
| H | i-Pr | c-Pr | Ph-4-O(L-45g) |
| H | i-Pr | c-Pr | Ph-3,4-Cl₂ |
| H | i-Pr | c-Pr | Ph(-3-OCF₂O-4-) |
| H | i-Pr | c-Pr | Ph(-3-OCF₂CF₂O-4-) |
| H | i-Pr | c-Pr | (L-45c)Br |
| H | i-Pr | c-Pr | (L-45c)CF₃ |
| H | i-Pr | c-Pr | L-45e |
| H | i-Pr | c-Pr | L-45f |
| H | i-Pr | c-Pr | (L-46c)Br |
| H | i-Pr | c-Pr | (L-46c)CF₃ |
| H | i-Pr | c-Pr | L-46d |
| H | i-Pr | n-Bu | Ph-4-Br |
| H | i-Pr | s-Bu | Ph-4-CF₃ |
| H | i-Pr | i-Bu | Ph-4-OCF₃ |
| H | i-Pr | c-Bu | (L-45c)CF₃ |
| H | i-Pr | n-Pen | Ph-4-Br |
| H | i-Pr | c-Pen | Ph-4-CF₃ |
| H | i-Pr | n-Hex | Ph-4-OCF₃ |
| H | i-Pr | c-Hex | (L-45c)CF₃ |
| H | i-Pr | CH₂F | Ph-4-Br |
| H | i-Pr | CH₂Cl | Ph-4-CF₃ |
| H | i-Pr | CH₂Br | Ph-4-OCF₃ |
| H | i-Pr | CHF₂ | CF₂O(Ph-4-Cl) |
| H | i-Pr | CHF₂ | CH═CH(Ph-4-Cl) |
| H | i-Pr | CHF₂ | CH═CH(Ph-4-OCF₃) |
| H | i-Pr | CHF₂ | CH═CH(Ph-3,4-Cl₂) |
| H | i-Pr | CHF₂ | Ph-4-F |
| H | i-Pr | CHF₂ | Ph-4-Cl |
| H | i-Pr | CHF₂ | Ph-4-Br |
| H | i-Pr | CHF₂ | Ph-4-I |
| H | i-Pr | CHF₂ | Ph-4-CF₃ |
| H | i-Pr | CHF₂ | Ph-4-OCHF₂ |
| H | i-Pr | CHF₂ | Ph-4-OCF₃ |
| H | i-Pr | CHF₂ | Ph-4-OCF₂Br |
| H | i-Pr | CHF₂ | Ph-4-OCF₂CHF₂ |
| H | i-Pr | CHF₂ | Ph-4-OCF₂CHFCl |
| H | i-Pr | CHF₂ | Ph-4-OCF₂CHFCF₃ |
| H | i-Pr | CHF₂ | Ph-4-OCF₂CHFOCF₃ |
| H | i-Pr | CHF₂ | Ph-4-OCF₂CHFOCF₂CF₂CF₃ |
| H | i-Pr | CHF₂ | Ph-4-O(L-45g) |
| H | i-Pr | CHF₂ | Ph-3,4-Cl₂ |
| H | i-Pr | CHF₂ | Ph(-3-OCF₂O-4-) |
| H | i-Pr | CHF₂ | Ph(-3-OCF₂CF₂O-4-) |
| H | i-Pr | CHF₂ | (L-45c)Cl |
| H | i-Pr | CHF₂ | (L-45c)Br |
| H | i-Pr | CHF₂ | (L-45c)CF₃ |
| H | i-Pr | CHF₂ | L-45e |
| H | i-Pr | CHF₂ | L-45f |
| H | i-Pr | CHF₂ | (L-46c)Cl |
| H | i-Pr | CHF₂ | (L-46c)Br |
| H | i-Pr | CHF₂ | (L-46c)CF₃ |
| H | i-Pr | CHF₂ | L-46d |
| H | i-Pr | CHFCl | (L-45c)CF₃ |
| H | i-Pr | CHFBr | Ph-4-Br |
| H | i-Pr | CF₃ | T-1 |
| H | i-Pr | CF₃ | T-2 |
| H | i-Pr | CF₃ | T-3 |
| H | i-Pr | CF₃ | T-4 |
| H | i-Pr | CF₃ | T-5 |
| H | i-Pr | CF₃ | CH₂OCH₃ |
| H | i-Pr | CF₃ | CH₂OEt |
| H | i-Pr | CF₃ | CH₂OPr-n |
| H | i-Pr | CF₃ | CH₂OPr-i |
| H | i-Pr | CF₃ | CH₂OBu-n |
| H | i-Pr | CF₃ | CH₂OCH₂CF₃ |
| H | i-Pr | CF₃ | CH₂OCH₂CF₂CF₃ |
| H | i-Pr | CF₃ | CH₂OCH(CF₃)₂ |
| H | i-Pr | CF₃ | CH₂OC(O)(Ph-2-Cl) |
| H | i-Pr | CF₃ | CH₂OC(O)(Ph-3-Cl) |
| H | i-Pr | CF₃ | CH₂OC(O)(Ph-4-Cl) |
| H | i-Pr | CF₃ | CH₂OPh |
| H | i-Pr | CF₃ | CH₂O(Ph-4-F) |
| H | i-Pr | CF₃ | CH₂O(Ph-2-Cl) |
| H | i-Pr | CF₃ | CH₂O(Ph-3-Cl) |
| H | i-Pr | CF₃ | CH₂O(Ph-4-Cl) |
| H | i-Pr | CF₃ | CH₂O(Ph-4-Br) |
| H | i-Pr | CF₃ | CH₂O(Ph-4-CF₃) |
| H | i-Pr | CF₃ | CH₂O(Ph-4-OCF₃) |
| H | i-Pr | CF₃ | CH₂CH₂OCH₃ |
| H | i-Pr | CF₃ | CF₂OPh |
| H | i-Pr | CF₃ | CF₂O(Ph-3-F) |
| H | i-Pr | CF₃ | CF₂O(Ph-4-F) |
| H | i-Pr | CF₃ | CF₂O(Ph-2-Cl) |
| H | i-Pr | CF₃ | CF₂O(Ph-3-Cl) |
| H | i-Pr | CF₃ | CF₂O(Ph-4-Cl) |
| H | i-Pr | CF₃ | CF₂O(Ph-3-Br) |
| H | i-Pr | CF₃ | CF₂O(Ph-4-Br) |
| H | i-Pr | CF₃ | CF₂O(Ph-3-CF₃) |
| H | i-Pr | CF₃ | CF₂O(Ph-4-CF₃) |
| H | i-Pr | CF₃ | CF₂O(Ph-3-OCF₃) |
| H | i-Pr | CF₃ | CF₂O(Ph-4-OCF₃) |
| H | i-Pr | CF₃ | M-4a |
| H | i-Pr | CF₃ | M-5a |
| H | i-Pr | CF₃ | CH₂SCH₃ |
| H | i-Pr | CF₃ | CH₂SO₂CH₃ |
| H | i-Pr | CF₃ | CH₂SCF₃ |
| H | i-Pr | CF₃ | CH₂SO₂CF₃ |
| H | i-Pr | CF₃ | CH₂SPh |
| H | i-Pr | CF₃ | CH₂S(Ph-3-Cl) |
| H | i-Pr | CF₃ | CH₂S(Ph-4-Cl) |
| H | i-Pr | CF₃ | CH₂SO₂(Ph-3-Cl) |
| H | i-Pr | CF₃ | CH₂SO₂(Ph-4-Cl) |
| H | i-Pr | CF₃ | CH₂CH₂SCH₃ |
| H | i-Pr | CF₃ | CH₂CH₂SO₂CH₃ |
| H | i-Pr | CF₃ | CH₂CH(CH₃)SCH₃ |
| H | i-Pr | CF₃ | CH₂CH(CH₃)SEt |
| H | i-Pr | CF₃ | CH₂CH₂SCF₃ |
| H | i-Pr | CF₃ | CH₂N(CH₃)₂ |
| H | i-Pr | CF₃ | CH₂NHPh |
| H | i-Pr | CF₃ | CH₂NH(Ph-3-Cl) |
| H | i-Pr | CF₃ | CH₂NH(Ph-4-Cl) |
| H | i-Pr | CF₃ | CF₂C(O)OEt |
| H | i-Pr | CF₃ | CH₂CH₂Ph |
| H | i-Pr | CF₃ | CH₂CH₂(Ph-3-Cl) |
| H | i-Pr | CF₃ | CH₂CH₂(Ph-4-Cl) |
| H | i-Pr | CF₃ | CH(CH₃)CH₂Ph |
| H | i-Pr | CF₃ | CH₂(L-5a) |
| H | i-Pr | CF₃ | CH₂(L-14a) |
| H | i-Pr | CF₃ | CH₂(L-24a) |
| H | i-Pr | CF₃ | CH₂(L-36a) |
| H | i-Pr | CF₃ | T-22 |
| H | i-Pr | CF₃ | T-23 |
| H | i-Pr | CF₃ | T-24 |
| H | i-Pr | CF₃ | C(O)OCH₃ |
| H | i-Pr | CF₃ | C(O)OEt |
| H | i-Pr | CF₃ | C(O)OPr-n |
| H | i-Pr | CF₃ | C(O)OPr-i |
| H | i-Pr | CF₃ | C(O)OBu-n |
| H | i-Pr | CF₃ | C(O)OBu-t |
| H | i-Pr | CF₃ | C(O)OCH₂CF₃ |
| H | i-Pr | CF₃ | C(O)SEt |
| H | i-Pr | CF₃ | C(O)N(CH₃)₂ |
| H | i-Pr | CF₃ | CH═CHPh |
| H | i-Pr | CF₃ | CH═CH(Ph-3-F) |
| H | i-Pr | CF₃ | CH═CH(Ph-4-F) |
| H | i-Pr | CF₃ | CH═CH(Ph-2-Cl) |
| H | i-Pr | CF₃ | CH═CH(Ph-3-Cl) |
| H | i-Pr | CF₃ | CH═CH(Ph-4-Cl) |
| H | i-Pr | CF₃ | CH═CH(Ph-3-Br) |
| H | i-Pr | CF₃ | CH═CH(Ph-4-Br) |
| H | i-Pr | CF₃ | CH═CH(Ph-3-CF₃) |
| H | i-Pr | CF₃ | CH═CH(Ph-4-CF₃) |
| H | i-Pr | CF₃ | CH═CH(Ph-3-OCF₃) |
| H | i-Pr | CF₃ | CH═CH(Ph-4-OCF₃) |
| H | i-Pr | CF₃ | CH═CH(Ph-3-SCH₃) |
| H | i-Pr | CF₃ | CH═CH(Ph-4-SCH₃) |
| H | i-Pr | CF₃ | CH═CH(Ph-3-SO₂CH₃) |
| H | i-Pr | CF₃ | CH═CH(Ph-4-SO₂CH₃) |
| H | i-Pr | CF₃ | CH═CH(Ph-3,4-F₂) |
| H | i-Pr | CF₃ | CH═CH(Ph-3-F-4-Cl) |
| H | i-Pr | CF₃ | CH═CH(Ph-3,4-Cl₂) |
| H | i-Pr | CF₃ | CH═CH(Ph-3,4-Br₂) |
| H | i-Pr | CF₃ | CH═CH(Ph-3-F-4-CF₃) |
| H | i-Pr | CF₃ | CH═CH(Ph-3-Cl-4-OCF₃) |
| H | i-Pr | CF₃ | CH═CH[Ph(-3-OCF₂O-4-)] |
| H | i-Pr | CF₃ | Ph |
| H | i-Pr | CF₃ | Ph-3-F |
| H | i-Pr | CF₃ | Ph-4-F |
| H | i-Pr | CF₃ | Ph-2-Cl |
| H | i-Pr | CF₃ | Ph-3-Cl |
| H | i-Pr | CF₃ | Ph-4-Cl |
| H | i-Pr | CF₃ | Ph-3-Br |
| H | i-Pr | CF₃ | Ph-4-Br |
| H | i-Pr | CF₃ | Ph-3-I |
| H | i-Pr | CF₃ | Ph-4-I |
| H | i-Pr | CF₃ | Ph-4-CH₃ |
| H | i-Pr | CF₃ | Ph-4-Et |
| H | i-Pr | CF₃ | Ph-4-Pr-n |
| H | i-Pr | CF₃ | Ph-4-Pr-i |
| H | i-Pr | CF₃ | Ph-4-Bu-n |
| H | i-Pr | CF₃ | Ph-4-Bu-i |
| H | i-Pr | CF₃ | Ph-4-Bu-t |
| H | i-Pr | CF₃ | Ph-3-CF₃ |
| H | i-Pr | CF₃ | Ph-4-CF₃ |
| H | i-Pr | CF₃ | Ph-4-C(CF₃)₂OH |
| H | i-Pr | CF₃ | Ph-4-C(CF₃)₂OCH₃ |
| H | i-Pr | CF₃ | Ph-4-CH₂OCH₃ |
| H | i-Pr | CF₃ | Ph-4-CH₂OCH₂CF₃ |
| H | i-Pr | CF₃ | Ph-4-CH₂SCH₃ |
| H | i-Pr | CF₃ | Ph-4-CH₂S(O)CH₃ |
| H | i-Pr | CF₃ | Ph-4-CH₂SO₂CH₃ |
| H | i-Pr | CF₃ | Ph-4-CH₂SEt |
| H | i-Pr | CF₃ | Ph-4-CH₂S(O)Et |
| H | i-Pr | CF₃ | Ph-4-CH₂SO₂Et |
| H | i-Pr | CF₃ | Ph-4-CH₂SPr-n |
| H | i-Pr | CF₃ | Ph-4-CH₂SO₂Pr-n |
| H | i-Pr | CF₃ | Ph-4-CH₂SPr-i |
| H | i-Pr | CF₃ | Ph-4-CH₂SO₂Pr-i |
| H | i-Pr | CF₃ | Ph-4-CH₂SPr-c |
| H | i-Pr | CF₃ | Ph-4-CH₂SO₂Pr-c |
| H | i-Pr | CF₃ | Ph-4-CH₂SBu-n |
| H | i-Pr | CF₃ | Ph-4-CH₂SO₂Bu-n |
| H | i-Pr | CF₃ | Ph-4-CH₂SCF₃ |
| H | i-Pr | CF₃ | Ph-4-CH₂S(O)CF₃ |
| H | i-Pr | CF₃ | Ph-4-CH₂SO₂CF₃ |
| H | i-Pr | CF₃ | Ph-4-CH₂SCH₂CF₃ |
| H | i-Pr | CF₃ | Ph-4-OH |
| H | i-Pr | CF₃ | Ph-4-OCH₃ |
| H | i-Pr | CF₃ | Ph-4-OEt |
| H | i-Pr | CF₃ | Ph-4-OPr-n |
| H | i-Pr | CF₃ | Ph-4-OPr-i |
| H | i-Pr | CF₃ | Ph-4-OBu-n |
| H | i-Pr | CF₃ | Ph-4-OBu-t |
| H | i-Pr | CF₃ | Ph-4-OCHF₂ |
| H | i-Pr | CF₃ | Ph-3-OCF₃ |
| H | i-Pr | CF₃ | Ph-4-OCF₃ |
| H | i-Pr | CF₃ | Ph-4-OCF₂Br |
| H | i-Pr | CF₃ | Ph-4-OCH₂CF3 |
| H | i-Pr | CF₃ | Ph-4-OCF₂CHF₂ |
| H | i-Pr | CF₃ | Ph-4-OCF₂CHFCl |
| H | i-Pr | CF₃ | Ph-4-OCF₂CHFBr |
| H | i-Pr | CF₃ | Ph-4-OCF₂CF₂Br |
| H | i-Pr | CF₃ | Ph-4-OCF₂CFCl₂ |
| H | i-Pr | CF₃ | Ph-4-OCF₂CCl₃ |
| H | i-Pr | CF₃ | Ph-4-OCH₂CF₂CHF₂ |
| H | i-Pr | CF₃ | Ph-3-OCF₂CHFCF₃ |
| H | i-Pr | CF₃ | Ph-4-OCF₂CHFCF₃ |
| H | i-Pr | CF₃ | Ph-4-OCH(CF₃)₂ |
| H | i-Pr | CF₃ | Ph-4-OCF₂CFBrCF₃ |
| H | i-Pr | CF₃ | Ph-3-OCF₂CHFOCF₃ |
| H | i-Pr | CF₃ | Ph-4-OCF₂CHFOCF₃ |
| H | i-Pr | CF₃ | Ph-4-OCF₂CHFOCF₂CF₂CF₃ |
| H | i-Pr | CF₃ | Ph-4-OCH₂CH═CH₂ |
| H | i-Pr | CF₃ | Ph-4-OCH₂CH═CF₂ |
| H | i-Pr | CF₃ | Ph-4-OCH₂CF═CF₂ |
| H | i-Pr | CF₃ | Ph-4-OCH₂CH═CCl₂ |
| H | i-Pr | CF₃ | Ph-4-OCH₂CCl═CCl₂ |
| H | i-Pr | CF₃ | Ph-4-OCH₂Ph |
| H | i-Pr | CF₃ | Ph-4-OSO₂CH₃ |
| H | i-Pr | CF₃ | Ph-4-OSO₂Et |
| H | i-Pr | CF₃ | Ph-4-OSO₂Pr-n |
| H | i-Pr | CF₃ | Ph-4-OSO₂Pr-i |
| H | i-Pr | CF₃ | Ph-4-OSO₂Bu-n |
| H | i-Pr | CF₃ | Ph-4-OSO₂CHCl₂ |
| H | i-Pr | CF₃ | Ph-4-OSO₂CF₃ |
| H | i-Pr | CF₃ | Ph-4-OSO₂CH₂CF₃ |
| H | i-Pr | CF₃ | Ph-3-O(Ph-4-Cl) |
| H | i-Pr | CF₃ | Ph-4-O(Ph-4-Cl) |
| H | i-Pr | CF₃ | Ph-4-O(Ph-4-Br) |
| H | i-Pr | CF₃ | Ph-4-O(Ph-4-CF₃) |
| H | i-Pr | CF₃ | Ph-4-O(L-21b)Br |
| H | i-Pr | CF₃ | Ph-4-O(L-21b)CF₃ |
| H | i-Pr | CF₃ | Ph-3-O(L-45c)Br |
| H | i-Pr | CF₃ | Ph-4-O(L-45c)Br |
| H | i-Pr | CF₃ | Ph-3-O(L-45c)CF₃ |
| H | i-Pr | CF₃ | Ph-4-O(L-45c)CF₃ |
| H | i-Pr | CF₃ | Ph-3-O(L-45e) |
| H | i-Pr | CF₃ | Ph-4-O(L-45e) |
| H | i-Pr | CF₃ | Ph-3-O(L-48b)Br |
| H | i-Pr | CF₃ | Ph-4-O(L-48b)Br |
| H | i-Pr | CF₃ | Ph-4-SCH₃ |
| H | i-Pr | CF₃ | Ph-4-S(O)CH₃ |
| H | i-Pr | CF₃ | Ph-4-SO₂CH₃ |
| H | i-Pr | CF₃ | Ph-4-SEt |
| H | i-Pr | CF₃ | Ph-4-S(O)Et |
| H | i-Pr | CF₃ | Ph-4-SO₂Et |
| H | i-Pr | CF₃ | Ph-4-SPr-n |
| H | i-Pr | CF₃ | Ph-4-S(O)Pr-n |
| H | i-Pr | CF₃ | Ph-4-SO₂Pr-n |
| H | i-Pr | CF₃ | Ph-4-SPr-i |
| H | i-Pr | CF₃ | Ph-4-S(O)Pr-i |
| H | i-Pr | CF₃ | Ph-4-SO₂Pr-i |
| H | i-Pr | CF₃ | Ph-4-SBu-n |
| H | i-Pr | CF₃ | Ph-4-S(O)Bu-n |
| H | i-Pr | CF₃ | Ph-4-SO₂Bu-n |
| H | i-Pr | CF₃ | Ph-4-SBu-t |
| H | i-Pr | CF₃ | Ph-4-S(O)Bu-t |
| H | i-Pr | CF₃ | Ph-4-SO₂Bu-t |
| H | i-Pr | CF₃ | Ph-4-SCH₂F |
| H | i-Pr | CF₃ | Ph-4-S(O)CH₂F |
| H | i-Pr | CF₃ | Ph-4-SO₂CH₂F |
| H | i-Pr | CF₃ | Ph-4-SCHF₂ |
| H | i-Pr | CF₃ | Ph-4-S(O)CHF₂ |
| H | i-Pr | CF₃ | Ph-4-SO₂CHF₂ |
| H | i-Pr | CF₃ | Ph-3-SCF₃ |
| H | i-Pr | CF₃ | Ph-4-SCF₃ |
| H | i-Pr | CF₃ | Ph-4-S(O)CF₃ |
| H | i-Pr | CF₃ | Ph-4-SO₂CF₃ |
| H | i-Pr | CF₃ | Ph-4-SCF₂Cl |
| H | i-Pr | CF₃ | Ph-4-S(O)CF₂Cl |
| H | i-Pr | CF₃ | Ph-4-SO₂CF₂Cl |
| H | i-Pr | CF₃ | Ph-4-SCF₂Br |
| H | i-Pr | CF₃ | Ph-4-S(O)CF₂Br |
| H | i-Pr | CF₃ | Ph-4-SO₂CF₂Br |
| H | i-Pr | CF₃ | Ph-3-S(Ph-4-Cl) |
| H | i-Pr | CF₃ | Ph-4-S(Ph-4-Cl) |
| H | i-Pr | CF₃ | Ph-4-S(Ph-4-Br) |
| H | i-Pr | CF₃ | Ph-4-S(Ph-4-CF₃) |
| H | i-Pr | CF₃ | Ph-4-S(L-45c)Br |
| H | i-Pr | CF₃ | Ph-4-S(L-45c)CF₃ |
| H | i-Pr | CF₃ | Ph-4-S(L-45e) |
| H | i-Pr | CF₃ | Ph-4-S(L-48b)Br |
| H | i-Pr | CF₃ | Ph-4-NO₂ |
| H | i-Pr | CF₃ | Ph-4-N(CH₃)₂ |
| H | i-Pr | CF₃ | Ph-4-N(Et)₂ |
| H | i-Pr | CF₃ | Ph-4-(T-16) |
| H | i-Pr | CF₃ | Ph-4-CN |
| H | i-Pr | CF₃ | Ph-4-C(O)OCH₃ |
| H | i-Pr | CF₃ | Ph-4-C(O)NH₂ |
| H | i-Pr | CF₃ | Ph-4-C(O)NHCH₃ |
| H | i-Pr | CF₃ | Ph-4-C(O)NHEt |
| H | i-Pr | CF₃ | Ph-4-C(O)N(CH₃)₂ |
| H | i-Pr | CF₃ | Ph-4-C(S)NH₂ |
| H | i-Pr | CF₃ | Ph-3-Ph |
| H | i-Pr | CF₃ | Ph-4-Ph |
| H | i-Pr | CF₃ | Ph-4-(L-5a) |
| H | i-Pr | CF₃ | Ph-4-(L-14a) |
| H | i-Pr | CF₃ | Ph-4-(L-24a) |
| H | i-Pr | CF₃ | Ph-4-(L-36a) |
| H | i-Pr | CF₃ | Ph-2, 4-F2 |
| H | i-Pr | CF₃ | Ph-3, 4-F2 |
| H | i-Pr | CF₃ | Ph-2-Cl-4-F |
| H | i-Pr | CF₃ | Ph-3-Cl-4-F |
| H | i-Pr | CF₃ | Ph-2-F-4-Cl |
| H | i-Pr | CF₃ | Ph-3-F-4-Cl |
| H | i-Pr | CF₃ | Ph-2,4-Cl₂ |
| H | i-Pr | CF₃ | Ph-3,4-Cl₂ |
| H | i-Pr | CF₃ | Ph-3,5-Cl₂ |
| H | i-Pr | CF₃ | Ph-3-Br-4-F |
| H | i-Pr | CF₃ | Ph-2-F-4-Br |
| H | i-Pr | CF₃ | Ph-3,4-Br₂ |
| H | i-Pr | CF₃ | Ph-3-CH₃-4-F |
| H | i-Pr | CF₃ | Ph-3-F-4-CH₃ |
| H | i-Pr | CF₃ | Ph-3,4-(CH₃)₂ |
| H | i-Pr | CF₃ | Ph-3-CF₃-4-F |
| H | i-Pr | CF₃ | Ph-3-CF₃-4-Cl |
| H | i-Pr | CF₃ | Ph-2-F-4-CF₃ |
| H | i-Pr | CF₃ | Ph-3-F-4-CF₃ |
| H | i-Pr | CF₃ | Ph-2-Cl-4-CF₃ |
| H | i-Pr | CF₃ | Ph-3-Br-4-OCH₃ |
| H | i-Pr | CF₃ | Ph-3-F-4-OCHF₂ |
| H | i-Pr | CF₃ | Ph-3-Cl-4-OCHF₂ |
| H | i-Pr | CF₃ | Ph-3-Br-4-OCHF₂ |
| H | i-Pr | CF₃ | Ph-3-F-4-OCF₃ |
| H | i-Pr | CF₃ | Ph-3-Cl-4-OCF₃ |
| H | i-Pr | CF₃ | Ph-3-Br-4-OCF₃ |
| H | i-Pr | CF₃ | Ph-3-F-4-OCF₂Br |
| H | i-Pr | CF₃ | Ph-3-Cl-4-OCF₂Br |
| H | i-Pr | CF₃ | Ph-3-Br-4-OCF₂Br |
| H | i-Pr | CF₃ | Ph-3-F-4-OCF₂CHF₂ |
| H | i-Pr | CF₃ | Ph-3-Cl-4-OCF₂CHF₂ |
| H | i-Pr | CF₃ | Ph-3-Br-4-OCF₂CHF₂ |
| H | i-Pr | CF₃ | Ph-3-F-4-OCF₂CHFCl |
| H | i-Pr | CF₃ | Ph-3-Cl-4-OCF₂CHFCl |
| H | i-Pr | CF₃ | Ph-3-Br-4-OCF₂CHFCl |
| H | i-Pr | CF₃ | Ph-3-F-4-OCF₂CHFCF₃ |
| H | i-Pr | CF₃ | Ph-3-Cl-4-OCF₂CHFCF₃ |
| H | i-Pr | CF₃ | Ph-3-Br-4-OCF₂CHFCF₃ |
| H | i-Pr | CF₃ | Ph-3-F-4-OCF₂CHFOCF₃ |
| H | i-Pr | CF₃ | Ph-3-Cl-4-OCF₂CHFOCF₃ |
| H | i-Pr | CF₃ | Ph-3-Br-4-OCF₂CHFOCF₃ |
| H | i-Pr | CF₃ | Ph-3-CH₃-4-OCF₂CHFOCF₃ |
| H | i-Pr | CF₃ | Ph-3-F-4-OCF₂CHFOCF₂CF₂CF₃ |
| H | i-Pr | CF₃ | Ph-3-Cl-4-OCF₂CHFOCF₂CF₂CF₃ |
| H | i-Pr | CF₃ | Ph-3-Br-4-OCF₂CHFOCF₂CF₂CF₃ |
| H | i-Pr | CF₃ | Ph(-3-OCF₂O-4-) |
| H | i-Pr | CF₃ | Ph(-3-OCF₂CF₂O-4-) |
| H | i-Pr | CF₃ | Ph-3-OPh-4-F |
| H | i-Pr | CF₃ | Ph-3-NO₂-4-F |
| H | i-Pr | CF₃ | Ph-3-NO₂-4-Cl |
| H | i-Pr | CF₃ | Ph-3-CN-4-F |
| H | i-Pr | CF₃ | Ph-2,3,4-F₃ |
| H | i-Pr | CF₃ | Ph-2,4,5-F₃ |
| H | i-Pr | CF₃ | Ph-3,4,5-F₃ |
| H | i-Pr | CF₃ | Ph-2,3-F₂-4-CH₃ |
| H | i-Pr | CF₃ | Ph-2,3-F₂-4-CF₃ |
| H | i-Pr | CF₃ | Ph-3,4-F₂-5-CF₃ |
| H | i-Pr | CF₃ | Ph-2-F-3-Cl-5-CF₃ |
| H | i-Pr | CF₃ | Ph-3,5-Cl₂-4-OCH₃ |
| H | i-Pr | CF₃ | 1-Naph |
| H | i-Pr | CF₃ | 2-Naph |
| H | i-Pr | CF₃ | L-1a |
| H | i-Pr | CF₃ | (L-1b)Br |
| H | i-Pr | CF₃ | (L-1c)Cl |
| H | i-Pr | CF₃ | (L-1c)Br |
| H | i-Pr | CF₃ | (L-1c)I |
| H | i-Pr | CF₃ | (L-1c)CF₃ |
| H | i-Pr | CF₃ | (L-1c)SCH₃ |
| H | i-Pr | CF₃ | (L-1c)SO₂CH₃ |
| H | i-Pr | CF₃ | (L-1c)NO₂ |
| H | i-Pr | CF₃ | L-2a |
| H | i-Pr | CF₃ | (L-2b)Br |
| H | i-Pr | CF₃ | L-3a |
| H | i-Pr | CF₃ | (L-3b)Ci |
| H | i-Pr | CF₃ | (L-3b)Br |
| H | i-Pr | CF₃ | (L-3b)SCH₃ |
| H | i-Pr | CF₃ | (L-3b)SO₂CH₃ |
| H | i-Pr | CF₃ | (L-3c)F |
| H | i-Pr | CF₃ | (L-3c)Cl |
| H | i-Pr | CF₃ | (L-3c)Br |
| H | i-Pr | CF₃ | (L-3c)I |
| H | i-Pr | CF₃ | (L-3c)CF₃ |
| H | i-Pr | CF₃ | (L-3c)SCH₃ |
| H | i-Pr | CF₃ | (L-3c)SO₂CH₃ |
| H | i-Pr | CF₃ | (L-3c)NO₂ |
| H | i-Pr | CF₃ | (L-3c)CN |
| H | i-Pr | CF₃ | L-3d |
| H | i-Pr | CF₃ | L-4a |
| H | i-Pr | CF₃ | (L-4b)Cl |
| H | i-Pr | CF₃ | (L-4b)Br |
| H | i-Pr | CF₃ | (L-4b)NO₂ |
| H | i-Pr | CF₃ | (L-4b)CN |
| H | i-Pr | CF₃ | (L-6a)Cl |
| H | i-Pr | CF₃ | (L-6a)Br |
| H | i-Pr | CF₃ | (L-6b)Cl |
| H | i-Pr | CF₃ | (L-6b)Br |
| H | i-Pr | CF₃ | (L-6b)NO₂ |
| H | i-Pr | CF₃ | L-8a |
| H | i-Pr | CF₃ | L-10a |
| H | i-Pr | CF₃ | (L-10b)Cl |
| H | i-Pr | CF₃ | (L-10b)Br |
| H | i-Pr | CF₃ | (L-10b)SCH₃ |
| H | i-Pr | CF₃ | L-11a |
| H | i-Pr | CF₃ | (L-15a)CHF₂ |
| H | i-Pr | CF₃ | (L-15b)CF₃ |
| H | i-Pr | CF₃ | (L-16a)CHF₂ |
| H | i-Pr | CF₃ | (L-16a)CF₂Br |
| H | i-Pr | CF₃ | (L-17a)Cl |
| H | i-Pr | CF₃ | L-19a |
| H | i-Pr | CF₃ | L-20a |
| H | i-Pr | CF₃ | L-21a |
| H | i-Pr | CF₃ | (L-21b)Cl |
| H | i-Pr | CF₃ | (L-21b)Br |
| H | i-Pr | CF₃ | (L-21b)I |
| H | i-Pr | CF₃ | (L-21b)CF₃ |
| H | i-Pr | CF₃ | (L-21b)NO₂ |
| H | i-Pr | CF₃ | L-22a |
| H | i-Pr | CF₃ | (L-22b)Cl |
| H | i-Pr | CF₃ | (L-22b)CF₃ |
| H | i-Pr | CF₃ | (L-22b)SCH₃ |
| H | i-Pr | CF₃ | L-23a |
| H | i-Pr | CF₃ | (L-23b)Cl |
| H | i-Pr | CF₃ | (L-23b)Br |
| H | i-Pr | CF₃ | (L-23b)SCH₃ |
| H | i-Pr | CF₃ | (L-23b)NO₂ |
| H | i-Pr | CF₃ | (L-23c)Cl |
| H | i-Pr | CF₃ | (L-23c)Br |
| H | i-Pr | CF₃ | (L-23c)SCH₃ |
| H | i-Pr | CF₃ | (L-25a)Cl |
| H | i-Pr | CF₃ | (L-25a)Br |
| H | i-Pr | CF₃ | (L-25a)I |
| H | i-Pr | CF₃ | (L-25a)CF₃ |
| H | i-Pr | CF₃ | L-25b |
| H | i-Pr | CF₃ | (L-30a)SCH₃ |
| H | i-Pr | CF₃ | (L-31a)Cl |
| H | i-Pr | CF₃ | (L-31a)Br |
| H | i-Pr | CF₃ | (L-31a)SCH₃ |
| H | i-Pr | CF₃ | (L-34a)Ph |
| H | i-Pr | CF₃ | (L-35a)Ph |
| H | i-Pr | CF₃ | (L-38a)Cl |
| H | i-Pr | CF₃ | (L-38a)Br |
| H | i-Pr | CF₃ | (L-38a)NO₂ |
| H | i-Pr | CF₃ | L-45a |
| H | i-Pr | CF₃ | (L-45b)Cl |
| H | i-Pr | CF₃ | (L-45c)F |
| H | i-Pr | CF₃ | (L-45c)Cl |
| H | i-Pr | CF₃ | (L-45c)Br |
| H | i-Pr | CF₃ | (L-45c)I |
| H | i-Pr | CF₃ | (L-45c)CF₃ |
| H | i-Pr | CF₃ | (L-45c)NO₂ |
| H | i-Pr | CF₃ | (L-45d)F |
| H | i-Pr | CF₃ | (L-45d)Cl |
| H | i-Pr | CF₃ | (L-45d)Br |
| H | i-Pr | CF₃ | L-45e |
| H | i-Pr | CF₃ | L-45f |
| H | i-Pr | CF₃ | L-46a |
| H | i-Pr | CF₃ | (L-46b)Br |
| H | i-Pr | CF₃ | (L-46c)F |
| H | i-Pr | CF₃ | (L-46c)Cl |
| H | i-Pr | CF₃ | (L-46c)Br |
| H | i-Pr | CF₃ | (L-46c)I |
| H | i-Pr | CF₃ | (L-46c)CF₃ |
| H | i-Pr | CF₃ | (L-46c)CH₂SCH₃ |
| H | i-Pr | CF₃ | (L-46c)CH₂SO₂CH₃ |
| H | i-Pr | CF₃ | (L-46c)OCH₂CF₃ |
| H | i-Pr | CF₃ | (L-46c)OCH(CF₃)₂ |
| H | i-Pr | CF₃ | (L-46c)OSO₂CH₃ |
| H | i-Pr | CF₃ | (L-4bc)SCH₃ |
| H | i-Pr | CF₃ | (L-46c)SO₂CH₃ |
| H | i-Pr | CF₃ | (L-46c)SEt |
| H | i-Pr | CF₃ | (L-46c)SPr-i |
| H | i-Pr | CF₃ | (L-46c)CN |
| H | i-Pr | CF₃ | L-46d |
| H | i-Pr | CF₃ | L-47a |
| H | i-Pr | CF₃ | (L-47b)F |
| H | i-Pr | CF₃ | (L-47b)Cl |
| H | i-Pr | CF₃ | L-47c |
| H | i-Pr | CF₃ | L-47d |
| H | i-Pr | CF₃ | L-48a |
| H | i-Pr | CF₃ | (L-48b)Br |
| H | i-Pr | CF₃ | L-50a |
| H | i-Pr | CF₃ | (L-50b)Cl |
| H | i-Pr | CF₃ | (L-50b)Br |
| H | i-Pr | CF₃ | (L-50b)I |
| H | i-Pr | CF₃ | (L-50b)SCH₃ |
| H | i-Pr | CF₃ | (L-50b)SO₂CH₃ |
| H | i-Pr | CF₃ | L-51a |
| H | i-Pr | CF₃ | (L-51b)Cl |
| H | i-Pr | CF₃ | (L-51b)SCH₃ |
| H | i-Pr | CF₃ | L-53a |
| H | i-Pr | CF₃ | L-55a |
| H | i-Pr | CF₂Cl | CF₂O(Ph-4-Cl) |
| H | i-Pr | CF₂Cl | CH═CH(Ph-4-Cl) |
| H | i-Pr | CF₂Cl | CH═CH(Ph-4-OCF₃) |
| H | i-Pr | CF₂Cl | CH═CH(Ph-3,4-Cl₂) |
| H | i-Pr | CF₂Cl | Ph-4-F |
| H | i-Pr | CF₂Cl | Ph-4-Cl |
| H | i-Pr | CF₂Cl | Ph-4-Br |
| H | i-Pr | CF₂Cl | Ph-4-I |
| H | i-Pr | CF₂Cl | Ph-4-CF₃ |
| H | i-Pr | CF₂Cl | Ph-4-OCHF₂ |
| H | i-Pr | CF₂Cl | Ph-4-OCF₃ |
| H | i-Pr | CF₂Cl | Ph-4-OCF₂Br |
| H | i-Pr | CF₂Cl | Ph-4-OCF₂CHF₂ |
| H | i-Pr | CF₂Cl | Ph-4-OCF₂CHFCl |
| H | i-Pr | CF₂Cl | Ph-4-OCF₂CHFCF₃ |
| H | i-Pr | CF₂Cl | Ph-4-OCF₂CHFOCF₃ |
| H | i-Pr | CF₂Cl | Ph-4-OCF₂CHFOCF₂CF₂CF₃ |
| H | i-Pr | CF₂Cl | Ph-4-O(L-45g) |
| H | i-Pr | CF₂Cl | Ph-3,4-Cl₂ |
| H | i-Pr | CF₂Cl | Ph(-3-OCF₂O-4-) |
| H | i-Pr | CF₂Cl | Ph(-3-OCF₂CF₂O-4-) |
| H | i-Pr | CF₂Cl | (L-45c)Cl |
| H | i-Pr | CF₂Cl | (L-45c)Br |
| H | i-Pr | CF₂Cl | (L-45c)CF₃ |
| H | i-Pr | CF₂Cl | L-45e |
| H | i-Pr | CF₂Cl | L-45f |
| H | i-Pr | CF₂Cl | (L-46c)Cl |
| H | i-Pr | CF₂Cl | (L-46c)Br |
| H | i-Pr | CF₂Cl | (L-46c)CF₃ |
| H | i-Pr | CF₂Cl | L-46d |
| H | i-Pr | CFCl₂ | Ph-4-CF₃ |
| H | i-Pr | CF₂Br | CF₂O(Ph-4-Cl) |
| H | i-Pr | CF₂Br | CH═CH(Ph-4-Cl) |
| H | i-Pr | CF₂Br | CH═CH(Ph-4-OCF₃) |
| H | i-Pr | CF₂Br | CH═CH(Ph-3,4-Cl₃) |
| H | i-Pr | CF₂Br | Ph-4-F |
| H | i-Pr | CF₂Br | Ph-4-Cl |
| H | i-Pr | CF₂Br | Ph-4-Br |
| H | i-Pr | CF₂Br | Ph-4-I |
| H | i-Pr | CF₂Br | Ph-4-CF₃ |
| H | i-Pr | CF₂Br | Ph-4-OCHF₂ |
| H | i-Pr | CF₂Br | Ph-4-OCF₃ |
| H | i-Pr | CF₂Br | Ph-4-OCF₂Br |
| H | i-Pr | CF₂Br | Ph-4-OCF₂CHF₂ |
| H | i-Pr | CF₂Br | Ph-4-OCF₂CHFCl |
| H | i-Pr | CF₂Br | Ph-4-OCF₂CHFCF₃ |
| H | i-Pr | CF₂Br | Ph-4-OCF₂CHFOCF₃ |
| H | i-Pr | CF₂Br | Ph-4-OCF₂CHFOCF₂CF₂CF₃ |
| H | i-Pr | CF₂Br | Ph-4-O(L-45g) |
| H | i-Pr | CF₂Br | Ph-3,4-Cl₂ |
| H | i-Pr | CF₂Br | Ph(-3-OCF₂O-4-) |
| H | i-Pr | CF₂Br | Ph(-3-OCF₂CF₂O-4-) |
| H | i-Pr | CF₂Br | (L-45c)Cl |
| H | i-Pr | CF₂Br | (L-45c)Br |
| H | i-Pr | CF₂Br | (L-45c)CF₃ |
| H | i-Pr | CF₂Br | L-45e |
| H | i-Pr | CF₂Br | L-45f |
| H | i-Pr | CF₂Br | (L-46c)Cl |
| H | i-Pr | CF₂Br | (L-46c)Br |
| H | i-Pr | CF₂Br | (L-46c)CF₃ |
| H | i-Pr | CF₂Br | L-46d |
| H | i-Pr | CFClBr | Ph-4-OCF₃ |
| H | i-Pr | CFBr₂ | (L-45c)CF₃ |
| H | i-Pr | CF₂CHF₂ | Ph-4-Br |
| H | i-Pr | CF₂CF₃ | CF₂O(Ph-4-Cl) |
| H | i-Pr | CF₂CF₃ | CH═CH(Ph-4-Cl) |
| H | i-Pr | CF₂CF₃ | CH═CH(Ph-4-OCF₃) |
| H | i-Pr | CF₂CF₃ | CH═CH(Ph-3,4-Cl₂) |
| H | i-Pr | CF₂CF₃ | Ph-4-F |
| H | i-Pr | CF₂CF₃ | Ph-4-Cl |
| H | i-Pr | CF₂CF₃ | Ph-4-Br |
| H | i-Pr | CF₂CF₃ | Ph-4-I |
| H | i-Pr | CF₂CF₃ | Ph-4-CF₃ |
| H | i-Pr | CF₂CF₃ | Ph-4-OCHF₂ |
| H | i-Pr | CF₂CF₃ | Ph-4-OCF₃ |
| H | i-Pr | CF₂CF₃ | Ph-4-OCF₂Br |
| H | i-Pr | CF₂CF₃ | Ph-4-OCF₂CHF₂ |
| H | i-Pr | CF₂CF₃ | Ph-4-OCF₂CHFCl |
| H | i-Pr | CF₂CF₃ | Ph-4-OCF₂CHFCF₃ |
| H | i-Pr | CF₂CF₃ | Ph-4-OCF₂CHFOCF₃ |
| H | i-Pr | CF₂CF₃ | Ph-4-OCF₂CHFOCF₂CF₂CF₃ |
| H | i-Pr | CF₂CF₃ | Ph-4-O(L-45g) |
| H | i-Pr | CF₂CF₃ | Ph-3,4-Cl₂ |
| H | i-Pr | CF₂CF₃ | Ph(-3-OCF₂O-4-) |
| H | i-Pr | CF₂CF₃ | Ph(-3-OCF₂CF₂O-4-) |
| H | i-Pr | CF₂CF₃ | (L-45c)Cl |
| H | i-Pr | CF₂CF₃ | (L-45c)Br |
| H | i-Pr | CF₂CF₃ | (L-45c)CF₃ |
| H | i-Pr | CF₂CF₃ | L-45e |
| H | i-Pr | CF₂CF₃ | L-45f |
| H | i-Pr | CF₂CF₃ | (L-46c)Cl |
| H | i-Pr | CF₂CF₃ | (L-46c)Br |
| H | i-Pr | CF₂CF₃ | (L-46c)CF₃ |
| H | i-Pr | CF₂CF₃ | L-46d |
| H | i-Pr | CF₂CF₂Cl | Ph-4-CF₃ |
| H | i-Pr | CFClCF₃ | Ph-4-OCF₃ |
| H | i-Pr | CFClCF₂Cl | (L-45c)CF₃ |
| H | i-Pr | CF₂CF₂Br | Ph-4-Br |
| H | i-Pr | CFBrCF₃ | Ph-4-CF₃ |
| H | i-Pr | CF₂CHFCF₃ | Ph-4-OCF₃ |
| H | i-Pr | CF₂CF₂CF₃ | CF₂O(Ph-4-Cl) |
| H | i-Pr | CF₂CF₂CF₃ | CH═CH(Ph-4-Cl) |
| H | i-Pr | CF₂CF₂CF₃ | CH═CH(Ph-4-OCF₃) |
| H | i-Pr | CF₂CF₂CF₃ | CH═CH(Ph-3,4-Cl₂) |
| H | i-Pr | CF₂CF₂CF₃ | Ph-4-F |
| H | i-Pr | CF₂CF₂CF₃ | Ph-4-Cl |
| H | i-Pr | CF₂CF₂CF₃ | Ph-4-Br |
| H | i-Pr | CF₂CF₂CF₃ | Ph-4-I |
| H | i-Pr | CF₂CF₂CF₃ | Ph-4-CF₃ |
| H | i-Pr | CF₂CF₂CF₃ | Ph-4-OCHF₂ |
| H | i-Pr | CF₂CF₂CF₃ | Ph-4-OCF₃ |
| H | i-Pr | CF₂CF₂CF₃ | Ph-4-OCF₂Br |
| H | i-Pr | CF₂CF₂CF₃ | Ph-4-OCF₂CHF₂ |
| H | i-Pr | CF₂CF₂CF₃ | Ph-4-OCF₂CHFCl |
| H | i-Pr | CF₂CF₂CF₃ | Ph-4-OCF₂CHFCF₃ |
| H | i-Pr | CF₂CF₂CF₃ | Ph-4-OCF₂CHFOCF₃ |
| H | i-Pr | CF₂CF₂CF₃ | Ph-4-OCF₂CHFOCF₂CF₂CF₃ |
| H | i-Pr | CF₂CF₂CF₃ | Ph-4-O(L-45g) |
| H | i-Pr | CF₂CF₂CF₃ | Ph-3,4-Cl₂ |
| H | i-Pr | CF₂CF₂CF₃ | Ph(-3-OCF₂O-4-) |
| H | i-Pr | CF₂CF₂CF₃ | Ph(-3-OCF₂CF₂O-4-) |
| H | i-Pr | CF₂CF₂CF₃ | (L-45c)Cl |
| H | i-Pr | CF₂CF₂CF₃ | (L-45c)Br |
| H | i-Pr | CF₂CF₂CF₃ | (L-45c)CF₃ |
| H | i-Pr | CF₂CF₂CF₃ | L-45e |
| H | i-Pr | CF₂CF₂CF₃ | L-45f |
| H | i-Pr | CF₂CF₂CF₃ | (L-46c)Cl |
| H | i-Pr | CF₂CF₂CF₃ | (L-46c)Br |
| H | i-Pr | CF₂CF₂CF₃ | (L-46c)CF₃ |
| H | i-Pr | CF₂CF₂CF₃ | L-46d |
| H | i-Pr | CF(CF₃)₂ | (L-45c)CF₃ |
| H | i-Pr | CF₂CFClCF₂Cl | Ph-40Br |
| H | i-Pr | CF₂CFBrCF₂Cl | Ph-4-CF₃ |
| H | i-Pr | CF₂CF₂CF₂CHF₂ | Ph-4-OCF₃ |
| H | i-Pr | CF₂CF₂CF₂CF₃ | (L-45c)CF₃ |
| H | i-Pr | CF(CF₃)CF₂CF₃ | Ph-4-Br |
| H | i-Pr | CF₂CF₂CF₂CF₂Cl | Ph-4-CF₃ |
| H | i-Pr | CF₂CF₂CF₂CF₂CF₂CF₃ | Ph-4-OCF₃ |
| H | i-Pr | T-1 | (L-45c)CF₃ |
| H | i-Pr | T-2 | Ph-4-Br |
| H | i-Pr | CH₂OCH₃ | Ph-4-CF₃ |
| H | i-Pr | CH₂OEt | Ph-4-OCF₃ |
| H | i-Pr | CH₂OCH₂CF₃ | (L-45c)CF₃ |
| H | i-Pr | CH₂OCH(CF₃)₂ | Ph-4-Br |
| H | i-Pr | CF₂OCH₃ | CF₂O(Ph-4-Cl) |
| H | i-Pr | CF₂OCH₃ | CH═CH(Ph-4-Cl) |
| H | i-Pr | CF₂OCH₃ | CH═CH(Ph-4-OCF₃) |
| H | i-Pr | CF₂OCH₃ | CH═CH(Ph-3,4-Cl₂) |
| H | i-Pr | CF₂OCH₃ | Ph-4-F |
| H | i-Pr | CF₂OCH₃ | Ph-4-Cl |
| H | i-Pr | CF₂OCH₃ | Ph-4-Br |
| H | i-Pr | CF₂OCH₃ | Ph-4-I |
| H | i-Pr | CF₂OCH₃ | Ph-4-CF₃ |
| H | i-Pr | CF₂OCH₃ | Ph-4-OCHF₂ |
| H | i-Pr | CF₂OCH₃ | Ph-4-OCF₃ |
| H | i-Pr | CF₂OCH₃ | Ph-4-OCF₂Br |
| H | i-Pr | CF₂OCH₃ | Ph-4-OCF₂CHF₂ |
| H | i-Pr | CF₂OCH₃ | Ph-4-OCF₂CHFCl |
| H | i-Pr | CF₂OCH₃ | Ph-4-OCF₂CHFCF₃ |
| H | i-Pr | CF₂OCH₃ | Ph-4-OCF₂CHFOCF₃ |
| H | i-Pr | CF₂OCH₃ | Ph-4-OCF₂CHFOCF₂CF₂CF₃ |
| H | i-Pr | CF₂OCH₃ | Ph-4-O(L-45g) |
| H | i-Pr | CF₂OCH₃ | Ph-3,4-Cl₂ |
| H | i-Pr | CF₂OCH₃ | Ph(-3-OCF₂O-4-) |
| H | i-Pr | CF₂OCH₃ | Ph(-3-OCF₂CF₂O-4-) |
| H | i-Pr | CF₂OCH₃ | (L-45c)Cl |
| H | i-Pr | CF₂OCH₃ | (L-45c)Br |
| H | i-Pr | CF₂OCH₃ | (L-45c)CF₃ |
| H | i-Pr | CF₂OCH₃ | L-45e |
| H | i-Pr | CF₂OCH₃ | L-45f |
| H | i-Pr | CF₂OCH₃ | (L-46c)Cl |
| H | i-Pr | CF₂OCH₃ | (L-46c)Br |
| H | i-Pr | CF₂OCH₃ | (L-46c)CF₃ |
| H | i-Pr | CF₂OCH₃ | L-46d |
| H | i-Pr | CF₂OCF₂CF₂CF₃ | Ph-4-CF₃ |
| H | i-Pr | CF₂OCF₂CF₂OCF₃ | Ph-4-OCF₃ |
| H | i-Pr | CF(CF₃)OCH₃ | (L-45c)CF₃ |
| H | i-Pr | CF(CF₃)OCF₂CF₂CF₃ | Ph-4-Br |
| H | i-Pr | CH₂SCH₃ | Ph-4-CF₃ |
| H | i-Pr | CH₂SO₂CH₃ | Ph-4-OCF₃ |
| H | i-Pr | CH₂SEt | (L-45c)CF₃ |
| H | i-Pr | CH₂SCF₃ | Ph-4-Br |
| H | i-Pr | CH₂SPh | Ph-4-CF₃ |
| H | i-Pr | CH₂CH₂SCH₃ | Ph-4-OCF₃ |
| H | i-Pr | CH₂CH₂SCF₃ | (L-45c)CF₃ |
| H | i-Pr | CF₂SCH₃ | CF₂O(Ph-4-Cl) |
| H | i-Pr | CF₂SCH₃ | CH═CH(Ph-4-Cl) |
| H | i-Pr | CF₂SCH₃ | CH═CH(Ph-4-OCF₃) |
| H | i-Pr | CF₂SCH₃ | CH═CH(Ph-3,4-Cl₂) |
| H | i-Pr | CF₂SCH₃ | Ph-4-F |
| H | i-Pr | CF₂SCH₃ | Ph-4-Cl |
| H | i-Pr | CF₂SCH₃ | Ph-4-Br |
| H | i-Pr | CF₂SCH₃ | Ph-4-I |
| H | i-Pr | CF₂SCH₃ | Ph-4-CF₃ |
| H | i-Pr | CF₂SCH₃ | Ph-4-OCHF₂ |
| H | i-Pr | CF₂SCH₃ | Ph-4-OCF₃ |
| H | i-Pr | CF₂SCH₃ | Ph-4-OCF₂Br |
| H | i-Pr | CF₂SCH₃ | Ph-4-OCF₂CHF₂ |
| H | i-Pr | CF₂SCH₃ | Ph-4-OCF₂CHFCl |
| H | i-Pr | CF₂SCH₃ | Ph-4-OCF₂CHFCF₃ |
| H | i-Pr | CF₂SCH₃ | Ph-4-OCF₂CHFOCF₃ |
| H | i-Pr | CF₂SCH₃ | Ph-4-OCF₂CHFOCF₂CF₂CF₃ |
| H | i-Pr | CF₂SCH₃ | Ph-4-O(L-45g) |
| H | i-Pr | CF₂SCH₃ | Ph-3,4-Cl₂ |
| H | i-Pr | CF₂SCH₃ | Ph(-3-OCF₂O-4-) |
| H | i-Pr | CF₂SCH₃ | Ph(-3-OCF₂CF₂O-4-) |
| H | i-Pr | CF₂SCH₃ | (L-45c)Cl |
| H | i-Pr | CF₂SCH₃ | (L-45c)Br |
| H | i-Pr | CF₂SCH₃ | (L-45c)CF₃ |
| H | i-Pr | CF₂SCH₃ | L-45e |
| H | i-Pr | CF₂SCH₃ | L-45f |
| H | i-Pr | CF₂SCH₃ | (L-46c)Cl |
| H | i-Pr | CF₂SCH₃ | (L-46c)Br |
| H | i-Pr | CF₂SCH₃ | (L-46c)CF₃ |
| H | i-Pr | CF₂SCH₃ | L-46d |
| H | i-Pr | CF₂SEt | Ph-4-Br |
| H | i-Pr | CF₂SPr-n | Ph-4-CF₃ |
| H | i-Pr | CF₂SPr-i | Ph-4-OCF₃ |
| H | i-Pr | CF₂SCH₂Ph | (L-45c)CF₃ |
| H | i-Pr | CF₂SPh | Ph-4-Br |
| H | i-Pr | CF₂C(O)OEt | Ph-4-CF₃ |
| H | i-Pr | CF₂SO₂N(CH₃)₂ | Ph-4-OCF₃ |
| H | i-Pr | CN | (L-45c)CF₃ |
| H | i-Pr | C(O) OCH3 | Ph-4-Br |
| H | i-Pr | C(O) OEt | Ph-4-CF₃ |
| H | i-Pr | C(O) OPr-n | Ph-4-OCF₃ |
| H | i-Pr | C(O) OPr-i | (L-45c)CF₃ |
| H | i-Pr | C(O) OBu-n | Ph-I-Br |
| H | i-Pr | C(O) OBu-t | Ph-4-CF₃ |
| H | i-Pr | C(O) SEt | Ph-4-OCF₃ |
| H | i-Pr | C(O) NH2 | (L-45c)CF₃ |
| H | i-Pr | C(S) NH2 | Ph-4-Br |
| H | i-Pr | C(CH₃)═NOCH₃ | Ph-I-CF₃ |
| H | i-Pr | Ph | Ph-4-OCF₃ |
| H | i-Pr | Ph-4-F | (L-45c)CF₃ |
| H | i-Pr | Ph-4-Cl | Ph-4-Br |
| H | i-Pr | Ph-4-CF₃ | Ph-I-CF₃ |
| H | i-Pr | Ph-4-OCF₃ | Ph-4-OCF₃ |
| CH₃ | i-Pr | CF₃ | Ph-4-Br |
| CH₃ | i-Pr | CF₃ | Ph-4-CF₃ |
| CH₃ | i-Pr | CF₃ | Ph-4-OCF₃ |
| CH₃ | i-Pr | CF₃ | (L-45c)CF₃ |
| Et | i-Pr | CF₃ | (L-45c)CF₃ |
| i-Pr | i-Pr | CF₃ | Ph-4-Br |
| CH₂OCH₃ | i-Pr | CF₃ | Ph-4-CF₃ |
| CH₂OEt | i-Pr | CF₃ | Ph-4-OCF₃ |
| H | c-Pr | CF₃ | CF₂O(Ph-4-Cl) |
| H | c-Pr | CF₃ | CH═CH(Ph-4-Cl) |
| H | c-Pr | CF₃ | CH═CH(Ph-4-OCF₃) |
| H | c-Pr | CF₃ | CH═CH(Ph-3,4-Cl₃) |
| H | c-Pr | CF₃ | Ph-4-F |
| H | c-Pr | CF₃ | Ph-4-Cl |
| H | c-Pr | CF₃ | Ph-4-Br |
| H | c-Pr | CF₃ | Ph-4-I |
| H | c-Pr | CF₃ | Ph-4-CF₃ |
| H | c-Pr | CF₃ | Ph-4-OCHF₂ |
| H | c-Pr | CF₃ | Ph-4-OCF₃ |
| H | c-Pr | CF₃ | Ph-4-OCF₂Br |
| H | c-Pr | CF₃ | Ph-4-OCF₂CHF₂ |
| H | c-Pr | CF₃ | Ph-4-OCF₂CHFCl |
| H | c-Pr | CF₃ | Ph-4-OCF₂CHFCF₃ |
| H | c-Pr | CF₃ | Ph-4-OCF₂CHFOCF₃ |
| H | c-Pr | CF₃ | Ph-4-OCF₂CHFOCF₂CF₂CF₃ |
| H | c-Pr | CF₃ | Ph-4-O(L-45g) |
| H | c-Pr | CF₃ | Ph-3,4-Cl₂ |
| H | c-Pr | CF₃ | Ph(-3-OCF₂O-4-) |
| H | c-Pr | CF₃ | Ph(-3-OCF₂CF₂O-4-) |
| H | c-Pr | CF₃ | (L-45c)Cl |
| H | c-Pr | CF₃ | (L-45c)Br |
| H | c-Pr | CF₃ | (L-45c)CF₃ |
| H | c-Pr | CF₃ | L-45e |
| H | c-Pr | CF₃ | L-45f |
| H | c-Pr | CF₃ | (L-46c)Cl |
| H | c-Pr | CF₃ | (L-46c)Br |
| H | c-Pr | CF₃ | (L-46c)CF₃ |
| H | c-Pr | CF₃ | L-46d |
| H | n-Bu | CF₃ | CH═CH(Ph-4-Cl) |
| H | n-Bu | CF₃ | Ph-4-Cl |
| H | n-Bu | CF₃ | Ph-4-Br |
| H | n-Bu | CF₃ | Ph-4-CF₃ |
| H | n-Bu | CF₃ | Ph-4-OCF₃ |
| H | n-Bu | CF₃ | Ph-4-OCF₂Br |
| H | n-Bu | CF₃ | Ph-4-OCF₂CHFCF₃ |
| H | n-Bu | CF₃ | Ph-4-OCF₂CHFOCF₃ |
| H | n-Bu | CF₃ | Ph-4-OCF₂CHFOCF₂CF₂CF₃ |
| H | n-Bu | CF₃ | Ph-4-O(L-45g) |
| H | n-Bu | CF₃ | Ph-3,4-Cl₂ |
| H | n-Bu | CF₃ | Ph(-3-OCF₂O-4-) |
| H | n-Bu | CF₃ | Ph(-3-OCF₂CF₂O-4-) |
| H | n-Bu | CF₃ | (L-45c)CF₃ |
| H | n-Bu | CF₃ | L-45f |
| H | n-Bu | CF₃ | (L-46c)CF₃ |
| H | n-Bu | CF₃ | L-46d |
| H | i-Bu | CF₃ | (L-45c)CF₃ |
| CH₃ | i-Bu | CF₃ | Ph-4-Br |
| H | CH₂Pr-c | CF₃ | Ph-4-CF₃ |
| H | s-Bu | CH₃ | Ph-4-Br |
| H | s-Bu | CH₃ | Ph-4-CF₃ |
| H | s-Bu | CH₃ | Ph-4-OCF₃ |
| H | s-Bu | CH₃ | (L-45c)Cl |
| H | s-Bu | CH₃ | (L-45c)Br |
| H | s-Bu | CH₃ | (L-45c)CF₃ |
| H | s-Bu | Et | Ph-4-OCF₃ |
| H | s-Bu | n-Pr | (L-45c)CF₃ |
| H | s-Bu | i-Pr | Ph-4-Br |
| H | s-Bu | c-Pr | Ph-4-CF₃ |
| H | s-Bu | CHF₂ | Ph-4-OCF₃ |
| H | s-Bu | CF₃ | CF₂O(Ph-4-Cl) |
| H | s-Bu | CF₃ | CH═CH(Ph-4-Cl) |
| H | s-Bu | CF₃ | CH═CH(Ph-4-OCF₃) |
| H | s-Bu | CF₃ | CH═CH(Ph-3,4-Cl₂) |
| H | s-Bu | CF₃ | Ph-4-F |
| H | s-Bu | CF₃ | Ph-4-Cl |
| H | s-Bu | CF₃ | Ph-4-Br |
| H | s-Bu | CF₃ | Ph-4-I |
| H | s-Bu | CF₃ | Ph-4-CF₃ |
| H | s-Bu | CF₃ | Ph-4-OCHF2 |
| H | s-Bu | CF₃ | Ph-4-OCF₃ |
| H | s-Bu | CF₃ | Ph-4-OCF₂Br |
| H | s-Bu | CF₃ | Ph-4-OCF₂CHF₂ |
| H | s-Bu | CF₃ | Ph-4-OCF₂CHFCl |
| H | s-Bu | CF₃ | Ph-4-OCF₂CHFCF₃ |
| H | s-Bu | CF₃ | Ph-4-OCF₂CHFOCF₃ |
| H | s-Bu | CF₃ | Ph-4-OCF₂CHFOCF₂CF₂CF₃ |
| H | s-Bu | CF₃ | Ph-4-O(L-45g) |
| H | s-Bu | CF₃ | Ph-3,4-Cl₂ |
| H | s-Bu | CF₃ | Ph(-3-OCF₂O-4-) |
| H | s-Bu | CF₃ | Ph(-3-OCF₂CF₂O-4-) |
| H | s-Bu | CF₃ | (L-45c)Cl |
| H | s-Bu | CF₃ | (L-45c)Br |
| H | s-Bu | CF₃ | (L-45c)CF₃ |
| H | s-Bu | CF₃ | L-45e |
| H | s-Bu | CF₃ | L-45f |
| H | s-Bu | CF₃ | (L-46c)Cl |
| H | s-Bu | CF₃ | (L-46c)Br |
| H | s-Bu | CF₃ | (L-46c)CF₃ |
| H | s-Bu | CF₃ | L-46d |
| H | s-Bu | CF₂Cl | (L-45c)CF₃ |
| H | s-Bu | CF₂Br | Ph-4-Br |
| H | s-Bu | CF₂CF₃ | Ph-4-CF₃ |
| H | s-Bu | CF₂CF₂CF₃ | Ph-4-OCF₃ |
| H | s-Bu | CF₂OCH₃ | (L-45c)CF₃ |
| H | s-Bu | CF₂SCH₃ | Ph-4-Br |
| H | t-Bu | CH₃ | Ph-4-Br |
| H | t-Bu | CH₃ | Ph-4-CF₃ |
| H | t-Bu | CH₃ | Ph-4-OCF₃ |
| H | t-Bu | CH₃ | (L-45c)Cl |
| H | t-Bu | CH₃ | (L-45c)Br |
| H | t-Bu | CH₃ | (L-45c)CF₃ |
| H | t-Bu | Et | Ph-4-CF₃ |
| H | t-Bu | n-Pr | Ph-4-OCF₃ |
| H | t-Bu | i-Pr | (L-45c)CF₃ |
| H | t-Bu | c-Pr | Ph-4-Br |
| H | t-Bu | CHF₂ | Ph-4-CF₃ |
| H | t-Bu | CF₃ | CF₂O(Ph-4-Cl) |
| H | t-Bu | CF₃ | CH═CH(Ph-4-Cl) |
| H | t-Bu | CF₃ | CH═CH(Ph-4-OCF₃) |
| H | t-Bu | CF₃ | CH═CH(Ph-3,4-Cl₂) |
| H | t-Bu | CF₃ | Ph-4-F |
| H | t-Bu | CF₃ | Ph-4-Cl |
| H | t-Bu | CF₃ | Ph-4-Br |
| H | t-Bu | CF₃ | Ph-4-I |
| H | t-Bu | CF₃ | Ph-4-CF₃ |
| H | t-Bu | CF₃ | Ph-4-OCHF₂ |
| H | t-Bu | CF₃ | Ph-4-OCF₃ |
| H | t-Bu | CF₃ | Ph-4-OCF₂Br |
| H | t-Bu | CF₃ | Ph-4-OCF₂CHF₂ |
| H | t-Bu | CF₃ | Ph-4-OCF₂CHFCl |
| H | t-Bu | CF₃ | Ph-4-OCF₂CHFCF₃ |
| H | t-Bu | CF₃ | Ph-4-OCF₂CHFOCF₃ |
| H | t-Bu | CF₃ | Ph-4-OCF₂CHFOCF₂CF₂CF₃ |
| H | t-Bu | CF₃ | Ph-4-O(L-45g) |
| H | t-Bu | CF₃ | Ph-3,4-Cl₂ |
| H | t-Bu | CF₃ | Ph(-3-OCF₂O-4-) |
| H | t-Bu | CF₃ | Ph(-3-OCF₂CF₂O-4-) |
| H | t-Bu | CF₃ | (L-45c)Cl |
| H | t-Bu | CF₃ | (L-45c)Br |
| H | t-Bu | CF₃ | (L-45c)CF₃ |
| H | t-Bu | CF₃ | L-45e |
| H | t-Bu | CF₃ | L-45f |
| H | t-Bu | CF₃ | (L-46c)Cl |
| H | t-Bu | CF₃ | (L-46c)Br |
| H | t-Bu | CF₃ | (L-46c)CF₃ |
| H | t-Bu | CF₃ | L-46d |
| H | t-Bu | CF₂Cl | Ph-4-OCF₃ |
| H | t-Bu | CF₂Br | (L-45c)CF₃ |
| H | t-Bu | CF₂CF₃ | Ph-4-Br |
| H | t-Bu | CF₂CF₂CF₃ | Ph-4-CF₃ |
| H | t-Bu | CF₂OCH₃ | Ph-4-OCF₃ |
| H | t-Bu | CF₂SCH₃ | (L-45c)CF₃ |
| H | c-Bu | CF3 | Ph-4-Br |
| H | n-Pen | CF3 | Ph-4-CF₃ |
| H | CH₂CH₂Pr-i | CF₃ | Ph-4-OCF₃ |
| H | CH₂CH(CH₃)Et | CF₃ | (L-45c)CF₃ |
| H | CH₂Bu-t | CF₃ | Ph-4-Br |
| H | CH(CH3)Pr-n | CF₃ | CF₂O(Ph-4-Cl) |
| H | CH(CH3)Pr-n | CF₃ | CH═CH(Ph-4-Cl) |
| H | CH(CH3)Pr-n | CF₃ | CH═CH(Ph-4-OCF₃) |
| H | CH(CH3)Pr-n | CF₃ | CH═CH(Ph-3,4-Cl₂) |
| H | CH(CH3)Pr-n | CF₃ | Ph-4-F |
| H | CH(CH3)Pr-n | CF₃ | Ph-4-Cl |
| H | CH(CH3)Pr-n | CF₃ | Ph-4-I |
| H | CH(CH3)Pr-n | CF₃ | Ph-4-CF₃ |
| H | CH(CH3)Pr-n | CF₃ | Ph-4-OCHF₂ |
| H | CH(CH3)Pr-n | CF₃ | Ph-4-OCF₃ |
| H | CH(CH3)Pr-n | CF₃ | Ph-4-OCF₂Br |
| H | CH(CH3)Pr-n | CF₃ | Ph-4-OCF₂CHF₂ |
| H | CH(CH3)Pr-n | CF₃ | Ph-4-OCF₂CHFCl |
| H | CH(CH3)Pr-n | CF₃ | Ph-4-OCF₂CHFCF₃ |
| H | CH(CH3)Pr-n | CF₃ | Ph-4-OCF₂CHFOCF₃ |
| H | CH(CH3)Pr-n | CF₃ | Ph-4-OCF₂CHFOCF₂CF₂CF₃ |
| H | CH(CH3)Pr-n | CF₃ | Ph-4-O(L-45g) |
| H | CH(CH3)Pr-n | CF₃ | Ph-3,4-Cl₂ |
| H | CH(CH3)Pr-n | CF₃ | Ph(-3-OCF₂O-4-) |
| H | CH(CH3)Pr-n | CF₃ | Ph(-3-OCF₂CF₂O-4-) |
| H | CH(CH3)Pr-n | CF₃ | (L-45c)Cl |
| H | CH(CH3)Pr-n | CF₃ | (L-45c)Br |
| H | CH(CH3)Pr-n | CF₃ | (L-45c)CF₃ |
| H | CH(CH3)Pr-n | CF₃ | L-45e |
| H | CH(CH3)Pr-n | CF₃ | L-45f |
| H | CH(CH3)Pr-n | CF₃ | (L-46c)Cl |
| H | CH(CH3)Pr-n | CF₃ | (L-46c)Br |
| H | CH(CH3)Pr-n | CF₃ | (L-46c)CF₃ |
| H | CH(CH3)Pr-n | CF₃ | L-46d |
| H | CH(CH3)Pr-i | CF₃ | Ph-4-CF₃ |
| H | CH(Et)₂ | CF₃ | Ph-4-OCF₃ |
| H | C(CH₃)₂Et | CF₃ | Ph-4-Br |
| H | C(CH₃)₂Et | CF₃ | Ph-4-CF₃ |
| H | C(CH₃)₂Et | CF₃ | Ph-4-OCF₃ |
| H | C(CH₃)₂Et | CF₃ | (L-45c)CF₃ |
| H | c-Pen | CF₃ | (L-45c)CF₃ |
| H | n-Hex | CF₃ | Ph-4-Br |
| H | CH(CH₃)Bu-i | CF₃ | Ph-4-CF₃ |
| H | C(CH₃)₂Pr-n | CF₃ | Ph-4-Br |
| H | C(CH₃)₂Pr-n | CF₃ | Ph-4-CF₃ |
| H | C(CH₃)₂Pr-n | CF₃ | Ph-4-OCF₃ |
| H | C(CH₃)₂Pr-n | CF₃ | (L-45c)CF₃ |
| H | c-Hex | CF₃ | Ph-4-OCF₃ |
| H | CH₂Hex-c | CF₃ | (L-45c)CF₃ |
| H | Oct | CF₃ | Ph-4-Br |
| H | C(CH₃)₂CH₂Bu-t | CF₃ | Ph-4-CF₃ |
| —CH₂CH₂CH₂CH₂— | CF₃ | Ph-4-OCF₃ | |
| —CH₂CH₂CH₂CH₂CH₂— | CF₃ | (L-45c)CF₃ | |
| —CH₂CH₂CH(CH₃)CH₂CH₂— | CF₃ | Ph-4-Br | |
| —CH₂CH(CH₃)CH₂CH(CH₃)CH₂— | CF₃ | Ph-4-CF₃ | |
| —CH₂CH₂CH₂CH₂CH₂CH₂— | CF₃ | Ph-4-OCF₃ | |
| H | CH₂CH₂F | CF₃ | (L-45c)CF₃ |
| H | CH₂CH₂Cl | CF₃ | Ph-4-Br |
| H | CH₂CF₃ | CF₃ | Ph-4-CF₃ |
| H | CH(CH₃)CH₂F | CF₃ | Ph-4-Br |
| H | CH(CH₃)CH₂F | CF₃ | Ph-4-CF₃ |
| H | CH(CH₃)CH₂F(S) | CF₃ | Ph-4-CF₃ |
| H | CH(CH₃)CH₂F | CF₃ | Ph-4-OCF₃ |
| H | CH(CH₃)CH₂F | CF₃ | (L-45c)CF₃ |
| H | CH(CH₃)CH₂Cl | CF₃ | Ph-4-Br |
| H | CH(CH₃)CH₂Cl | CF₃ | Ph-4-CF₃ |
| H | CH(CH₃)CH₂Cl | CF₃ | Ph-4-OCF₃ |
| H | CH(CH₃)CH₂Cl | CF₃ | (L-45c)CF₃ |
| H | CH(CH₃)CH₂Br | CF₃ | Ph-4-OCF₃ |
| H | CH(CH₃)CH₂Br(R) | CF₃ | Ph-4-OCF₃ |
| H | CH(CH₃)CH₂Br(S) | CF₃ | Ph-4-OCF₃ |
| H | C(CH₃)₂CH₂Cl | CF₃ | Ph-4-Br |
| H | C(CH₃)₂CH₂Cl | CF₃ | Ph-4-CF₃ |
| H | C(CH₃)₂CH₂Cl | CF₃ | Ph-4-OCF₃ |
| H | C(CH₃)₂CH₂Cl | CF₃ | (L-45c)CF₃ |
| H | C(CH₃)₂CH₂Br | CF₃ | (L-45c)CF₃ |
| H | C(CH₃)₂CHBrCH₂Br | CF₃ | Ph-4-Br |
| H | CH₂OCH₃ | CF₃ | Ph-4-CF₃ |
| H | CH₂CH₂OCH₃ | CF₃ | Ph-4-OCF₃ |
| H | CH₂CH₂OEt | CF₃ | (L-45c)CF₃ |
| CH₂CH₂OEt | CH₂CH₂OEt | CF₃ | Ph-4-Br |
| H | CH₂CH₂OC(O)NHEt | CF₃ | Ph-4-CF₃ |
| H | CH₂CH₂OPh | CF₃ | Ph-4-OCF₃ |
| H | CH₂CH₂O(Ph-2-Cl) | CF₃ | (L-45c)CF₃ |
| H | CH₂CH₂O(Ph-3-Cl) | CF₃ | Ph-4-Br |
| H | CH₂CH₂O(Ph-4-Cl) | CF₃ | Ph-4-CF₃ |
| H | CH₂CH(OH)CH₃ | CF₃ | Ph-4-OCF₃ |
| H | CH₂CH(OH)Et | CF₃ | (L-45c)CF₃ |
| H | CH₂CH(OH)Ph | CF₃ | Ph-4-Br |
| H | CH₂CH(OH)CH₂Ph | CF₃ | Ph-4-CF₃ |
| H | CH₂C(CH₃)₂OSi(CH₃)₃ | CF₃ | CH═CH(Ph-4-Cl) |
| H | CH₂C(CH₃)₂OSi(CH₃)₃ | CF₃ | Ph-4-Cl |
| H | CH₂C(CH₃)₂OSi(CH₃)₃ | CF₃ | Ph-4-Br |
| H | CH₂C(CH₃)₂OSi(CH₃)₃ | CF₃ | Ph-4-CF₃ |
| H | CH₂C(CH₃)₂OSi(CH₃)₃ | CF₃ | Ph-4-OCF₃ |
| H | CH₂C(CH₃)₂OSi(CH₃)₃ | CF₃ | Ph-4-OCF₂Br |
| H | CH₂C(CH₃)₂OSi(CH₃)₃ | CF₃ | Ph-4-OCF₂CHFCF₃ |
| H | CH₂C(CH₃)₂OSi(CH₃)₃ | CF₃ | Ph-4-OCF₂CHFOCF₃ |
| H | CH₂C(CH₃)₂OSi(CH₃)₃ | CF₃ | Ph-4-OCF₂CHFOCF₂CF₂CF₃ |
| H | CH₂C(CH₃)₂OSi(CH₃)₃ | CF₃ | Ph-4-O(L-45g) |
| H | CH₂C(CH₃)₂OSi(CH₃)₃ | CF₃ | Ph-3,4-Cl₂ |
| H | CH₂C(CH₃)₂OSi(CH₃)₃ | CF₃ | Ph(-3-OCF₂O-4-) |
| H | CH₂C(CH₃)₂OSi(CH₃)₃ | CF₃ | Ph(-3-OCF₂CF₂O-4-) |
| H | CH₂C(CH₃)₂OSi(CH₃)₃ | CF₃ | (L-45c)CF₃ |
| H | CH₂C(CH₃)₂OSi(CH₃)₃ | CF₃ | L-45f |
| H | CH₂C(CH₃)₂OSi(CH₃)₃ | CF₃ | (L-46c)CF₃ |
| H | CH₂C(CH₃)₂OSi(CH₃)₃ | CF₃ | L-46d |
| H | CH₂CH(OEt)₂ | CF₃ | Ph-4-OCF₃ |
| H | CH(CH₃)CH₂OH | CF₃ | (L-45c)CF₃ |
| H | CH(CH₃)CH₂OH(R) | CF₃ | (L-45c)CF₃ |
| H | CH(CH₃)CH₂OH(S) | CF₃ | (L-45c)CF₃ |
| H | CH(CH₃)CH₂OCH₃ | CH₃ | Ph-4-Br |
| H | CH(CH₃)CH₂OCH₃ | CH₃ | Ph-4-CF₃ |
| H | CH(CH₃)CH₂OCH₃ | CH₃ | Ph-4-OCF₃ |
| H | CH(CH₃)CH₂OCH₃ | CH₃ | (L-45c)Cl |
| H | CH(CH₃)CH₂OCH₃ | CH₃ | (L-45c)Br |
| H | CH(CH₃)CH₂OCH₃ | CH₃ | (L-45c)CF₃ |
| H | CH(CH₃)CH₂OCH₃ | Et | Ph-4-Br |
| H | CH(CH₃)CH₂OCH₃ | n-Pr | Ph-4-CF₃ |
| H | CH(CH₃)CH₂OCH₃ | i-Pr | Ph-4-OCF₃ |
| H | CH(CH₃)CH₂OCH₃ | c-Pr | (L-45c)CF₃ |
| H | CH(CH₃)CH₂OCH₃ | CHF₂ | Ph-4-Br |
| H | CH(CH₃)CH₂OCH₃ | CF₃ | CF₂O(Ph-4-Cl) |
| H | CH(CH₃)CH₂OCH₃ | CF₃ | CH═CH(Ph-4-Cl) |
| H | CH(CH₃)CH₂OCH₃ | CF₃ | CH═CH(Ph-4-OCF₃) |
| H | CH(CH₃)CH₂OCH₃ | CF₃ | CH═CH(Ph-3,4-Cl₂) |
| H | CH(CH₃)CH₂OCH₃ | CF₃ | Ph-4-F |
| H | CH(CH₃)CH₂OCH₃ | CF₃ | Ph-4-Cl |
| H | CH(CH₃)CH₂OCH₃ | CF₃ | Ph-4-Br |
| H | CH(CH₃)CH₂OCH₃ | CF₃ | Ph-4-I |
| H | CH(CH₃)CH₂OCH₃ | CF₃ | Ph-4-CF₃ |
| H | CH(CH₃)CH₂OCH₃ | CF₃ | Ph-4-OCHF₂ |
| H | CH(CH₃)CH₂OCH₃ | CF₃ | Ph-4-OCF₃ |
| H | CH(CH₃)CH₂OCH₃ | CF₃ | Ph-4-OCF₂Br |
| H | CH(CH₃)CH₂OCH₃ | CF₃ | Ph-4-OCF₂CHF₂ |
| H | CH(CH₃)CH₂OCH₃ | CF₃ | Ph-4-OCF₂CHFCl |
| H | CH(CH₃)CH₂OCH₃ | CF₃ | Ph-4-OCF₂CHFCF₃ |
| H | CH(CH₃)CH₂OCH₃ | CF₃ | Ph-4-OCF₂CHFOCF₃ |
| H | CH(CH₃)CH₂OCH₃ | CF₃ | Ph-4-OCF₂CHFOCF₂CF₂CF₂ |
| H | CH(CH₃)CH₂OCH₃ | CF₃ | Ph-4-O(L-45g) |
| H | CH(CH₃)CH₂OCH₃ | CF₃ | Ph-3,4-Cl₂ |
| H | CH(CH₃)CH₂OCH₃ | CF₃ | Ph(-3-OCF₂O-4-) |
| H | CH(CH₃)CH₂OCH₃ | CF₃ | Ph(-3-OCF₂CF₂O-4-) |
| H | CH(CH₃)CH₂OCH₃ | CF₃ | (L-45c)Cl |
| H | CH(CH₃)CH₂OCH₃ | CF₃ | (L-45c)Br |
| H | CH(CH₃)CH₂OCH₃ | CF₃ | (L-45c)CF₃ |
| H | CH(CH₃)CH₂OCH₃ | CF₃ | L-45e |
| H | CH(CH₃)CH₂OCH₃ | CF₃ | L-45f |
| H | CH(CH₃)CH₂OCH₃ | CF₃ | (L-46c)Cl |
| H | CH(CH₃)CH₂OCH₃ | CF₃ | (L-46c)Br |
| H | CH(CH₃)CH₂OCH₃ | CF₃ | (L-46c)CF₃ |
| H | CH(CH₃)CH₂OCH₃ | CF₃ | L-46d |
| H | CH(CH₃)CH₂OCH₃ | CF₂Cl | Ph-4-CF₃ |
| H | CH(CH₃)CH₂OCH₃ | CF₂Br | Ph-4-OCF₃ |
| H | CH(CH₃)CH₂OCH₃ | CF₂CF₃ | (L-45c)CF₃ |
| H | CH(CH₃)CH₂OCH₃ | CF₂CF₂CF₃ | Ph-4-Br |
| H | CH(CH₃)CH₂OCH₃ | CF₂OCH₃ | Ph-4-CF₃ |
| H | CH(CH₃)CH₂OCH₃ | CF₂SCH₃ | Ph-4-OCF₃ |
| H | CH(CH₃)CH₂OEt | CF₃ | (L-45c)CF₃ |
| H | CH(CH₃)CH₂OPr-n | CF₃ | Ph-4-Br |
| H | CH(CH₃)CH₂OBu-i | CF₃ | Ph-4-CF₃ |
| H | CH(CH₃)CH₂OCH₂CH₂OCH₃ | CF₃ | Ph-4-OCF₃ |
| H | CH(CH₃)CH₂OCH₂CH₂SCH₃ | CF₃ | (L-45c)CF₃ |
| H | CH(CH₃)CH₂OCH₂CH₂SEt | CF₃ | Ph-4-Br |
| H | CH(CH₃)CH₂OCH₂Ph | CF₃ | Ph-4-CF₃ |
| H | CH(CH₃)CH₂OC(O)CH₃ | CF₃ | CH═CH(Ph-4-Cl) |
| H | CH(CH₃)CH₂OC(O)CH₃ | CF₃ | Ph-4-Cl |
| H | CH(CH₃)CH₂OC(O)CH₃ | CF₃ | Ph-4-Br |
| H | CH(CH₃)CH₂OC(O)CH₃ | CF₃ | Ph-4-CF₃ |
| H | CH(CH₃)CH₂OC(O)CH₃ | CF₃ | Ph-4-OCF₃ |
| H | CH(CH₃)CH₂OC(O)CH₃ | CF₃ | Ph-4-OCF₂Br |
| H | CH(CH₃)CH₂OC(O)CH₃ | CF₃ | Ph-4-OCF₂CHFCF₃ |
| H | CH(CH₃)CH₂OC(O)CH₃ | CF₃ | Ph-4-OCF₂CHFOCF₃ |
| H | CH(CH₃)CH₂OC(O)CH₃ | CF₃ | Ph-4-OCF₂CHFOCF₂CF₂CF₃ |
| H | CH(CH₃)CH₂OC(O)CH₃ | CF₃ | Ph-4-O(L-45g) |
| H | CH(CH₃)CH₂OC(O)CH₃ | CF₃ | Ph-3,4-Cl₂ |
| H | CH(CH₃)CH₂OC(O)CH₃ | CF₃ | Ph(-3-OCF₂O-4-) |
| H | CH(CH₃)CH₂OC(O)CH₃ | CF₃ | Ph(-3-OCF₂CF₂O-4-) |
| H | CH(CH₃)CH₂OC(O)CH₃ | CF₃ | (L-45c)CF₃ |
| H | CH(CH₃)CH₂OC(O)CH₃ | CF₃ | L-45f |
| H | CH(CH₃)CH₂OC(O)CH₃ | CF₃ | (L-46c)CF₃ |
| H | CH(CH₃)CH₂OC(O)CH₃ | CF₃ | L-46d |
| H | CH(CH₃)CH₂OC(O)CF₃ | CF₃ | Ph-4-OCF₃ |
| H | CH(CH₃)CH₂OC(O)NHCH₃ | CF₃ | CF₂O(Ph-4-Cl) |
| H | CH(CH₃)CH₂OC(O)NHCH₃ | CF₃ | CH═CH(Ph-4-Cl) |
| H | CH(CH₃)CH₂OC(O)NHCH₃ | CF₃ | CH═CH(Ph-4-Br) |
| H | CH(CH₃)CH₂OC(O)NHCH₃ | CF₃ | CH═CH(Ph-4-CF₃) |
| H | CH(CH₃)CH₂OC(O)NHCH₃ | CF₃ | CH═CH(Ph-4-OCF₃) |
| H | CH(CH₃)CH₂OC(O)NHCH₃ | CF₃ | CH═CH(Ph-3,4-Cl₂) |
| H | CH(CH₃)CH₂OC(O)NHCH₃ | CF₃ | Ph-4-F |
| H | CH(CH₃)CH₂OC(O)NHCH₃ | CF₃ | Ph-4-Cl |
| H | CH(CH₃)CH₂OC(O)NHCH₃ | CF₃ | Ph-4-Br |
| H | CH(CH₃)CH₂OC(O)NHCH₃ | CF₃ | Ph-4-CF₃ |
| H | CH(CH₃)CH₂OC(O)NHCH₃ | CF₃ | Ph-4-OCF₃ |
| H | CH(CH₃)CH₂OC(O)NHCH₃ | CF₃ | Ph-4-OCF₂Br |
| H | CH(CH₃)CH₂OC(O)NHCH₃ | CF₃ | Ph-4-OCF₂CHF₂ |
| H | CH(CH₃)CH₂OC(O)NHCH₃ | CF₃ | Ph-4-OCF₂CHFCF₃ |
| H | CH(CH₃)CH₂OC(O)NHCH₃ | CF₃ | Ph-4-OCF₂CHFOCF₃ |
| H | CH(CH₃)CH₂OC(O)NHCH₃ | CF₃ | Ph-4-OCF₂CHFOCF₂CF₂CF₃ |
| H | CH(CH₃)CH₂OC(O)NHCH₃ | CF₃ | Ph-4-O(L-45g) |
| H | CH(CH₃)CH₂OC(O)NHCH₃ | CF₃ | Ph-3,4-F₂ |
| H | CH(CH₃)CH₂OC(O)NHCH₃ | CF₃ | Ph-3-F-4-Cl |
| H | CH(CH₃)CH₂OC(O)NHCH₃ | CF₃ | Ph-3,4-Cl₂ |
| H | CH(CH₃)CH₂OC(O)NHCH₃ | CF₃ | Ph(-3-OCF₂O-4-) |
| H | CH(CH₃)CH₂OC(O)NHCH₃ | CF₃ | Ph(-3-OCF₂CF₂O-4-) |
| H | CH(CH₃)CH₂OC(O)NHCH₃ | CF₃ | (L-45c)CF₃ |
| H | CH(CH₃)CH₂OC(O)NHCH₃ | CF₃ | L-45f |
| H | CH(CH₃)CH₂OC(O)NHCH₃ | CF₃ | (L-46c)CF₃ |
| H | CH(CH₃)CH₂OC(O)NHCH₃ | CF₃ | L-46d |
| H | CH(CH₃)CH₂OC(O)NHEt | CH₃ | Ph-4-Br |
| H | CH(CH₃)CH₂OC(O)NHEt | CH₃ | Ph-4-CF₃ |
| H | CH(CH₃)CH₂OC(O)NHEt | CH₃ | Ph-4-OCF₃ |
| H | CH(CH₃)CH₂OC(O)NHEt | CH₃ | (L-45c)Cl |
| H | CH(CH₃)CH₂OC(O)NHEt | CH₃ | (L-45c)Br |
| H | CH(CH₃)CH₂OC(O)NHEt | CH₃ | (L-45c)CF₃ |
| H | CH(CH₃)CH₂OC(O)NHEt | Et | (L-45c)CF₃ |
| H | CH(CH₃)CH₂OC(O)NHEt | n-Pr | Ph-4-Br |
| H | CH(CH₃)CH₂OC(O)NHEt | i-Pr | Ph-4-CF₃ |
| H | CH(CH₃)CH₂OC(O)NHEt | c-Pr | Ph-4-OCF₃ |
| H | CH(CH₃)CH₂OC(O)NHEt | CHF₂ | (L-45c)CF₃ |
| H | CH(CH₃)CH₂OC(O)NHEt | CF₃ | CF₂O(Ph-4-Cl) |
| H | CH(CH₃)CH₂OC(O)NHEt | CF₃ | CH═CH(Ph-4-Cl) |
| H | CH(CH₃)CH₂OC(O)NHEt | CF₃ | CH═CH(Ph-4-Br) |
| H | CH(CH₃)CH₂OC(O)NHEt | CF₃ | CH═CH(Ph-4-CF₃) |
| H | CH(CH₃)CH₂OC(O)NHEt | CF₃ | CH═CH(Ph-4-OCF₃) |
| H | CH(CH₃)CH₂OC(O)NHEt | CF₃ | CH═CH(Ph-3,4-Cl₂) |
| H | CH(CH₃)CH₂OC(O)NHEt | CF₃ | Ph-4-F |
| H | CH(CH₃)CH₂OC(O)NHEt | CF₃ | Ph-4-Cl |
| H | CH(CH₃)CH₂OC(O)NHEt | CF₃ | Ph-4-Br |
| H | CH(CH₃)CH₂OC(O)NHEt | CF₃ | Ph-4-I |
| H | CH(CH₃)CH₂OC(O)NHEt | CF₃ | Ph-4-CF₃ |
| H | CH(CH₃)CH₂OC(O)NHEt | CF₃ | Ph-4-OCHF₂ |
| H | CH(CH₃)CH₂OC(O)NHEt | CF₃ | Ph-4-OCF₃ |
| H | CH(CH₃)CH₂OC(O)NHEt | CF₃ | Ph-4-OCF₂Br |
| H | CH(CH₃)CH₂OC(O)NHEt | CF₃ | Ph-4-OCF₂CHF₂ |
| H | CH(CH₃)CH₂OC(O)NHEt | CF₃ | Ph-4-OCF₂CHFCl |
| H | CH(CH₃)CH₂OC(O)NHEt | CF₃ | Ph-4-OCF₂CHFCF₃ |
| H | CH(CH₃)CH₂OC(O)NHEt | CF₃ | Ph-4-OCF₂CHFOCF₃ |
| H | CH(CH₃)CH₂OC(O)NHEt | CF₃ | Ph-4-OCF₂CHFOCF₂CF₂CF₃ |
| H | CH(CH₃)CH₂OC(O)NHEt | CF₃ | Ph-4-O(L-45g) |
| H | CH(CH₃)CH₂OC(O)NHEt | CF₃ | Ph-3,4-F₂ |
| H | CH(CH₃)CH₂OC(O)NHEt | CF₃ | Ph-3-F-4-Cl |
| H | CH(CH₃)CH₂OC(O)NHEt | CF₃ | Ph-3,4-Cl₂ |
| H | CH(CH₃)CH₂OC(O)NHEt | CF₃ | Ph(-3-OCF₂O-4-) |
| H | CH(CH₃)CH₂OC(O)NHEt | CF₃ | Ph(-3-OCF₂CF₂O-4-) |
| H | CH(CH₃)CH₂OC(O)NHEt | CF₃ | (L-45c)Cl |
| H | CH(CH₃)CH₂OC(O)NHEt | CF₃ | (L-45c)Br |
| H | CH(CH₃)CH₂OC(O)NHEt | CF₃ | (L-45c)CF₃ |
| H | CH(CH₃)CH₂OC(O)NHEt | CF₃ | L-45e |
| H | CH(CH₃)CH₂OC(O)NHEt | CF₃ | L-45f |
| H | CH(CH₃)CH₂OC(O)NHEt | CF₃ | (L-46c)Cl |
| H | CH(CH₃)CH₂OC(O)NHEt | CF₃ | (L-46c)Br |
| H | CH(CH₃)CH₂OC(O)NHEt | CF₃ | (L-46c)CF₃ |
| H | CH(CH₃)CH₂OC(O)NHEt | CF₃ | L-46d |
| H | CH(CH₃)CH₂OC(O)NHEt | CF₂Cl | Ph-4-Br |
| H | CH(CH₃)CH₂OC(O)NHEt | CF₂Br | Ph-4-CF₃ |
| H | CH(CH₃)CH₂OC(O)NHEt | CF₂CF₃ | Ph-4-OCF₃ |
| H | CH(CH₃)CH₂OC(O)NHEt | CF₂CF₂CF₃ | (L-45c)CF₃ |
| H | CH(CH₃)CH₂OC(O)NHEt | CF₂OCH₃ | Ph-4-Br |
| H | CH(CH₃)CH₂OC(O)NHEt | CF₂SCH₃ | Ph-4-CF₃ |
| H | CH(CH₃)CH₂OC(O)NHPr-n | CF₃ | CH═CH(Ph-4-Cl) |
| H | CH(CH₃)CH₂OC(O)NHPr-n | CF₃ | Ph-4-Cl |
| H | CH(CH₃)CH₂OC(O)NHPr-n | CF₃ | Ph-4-Br |
| H | CH(CH₃)CH₂OC(O)NHPr-n | CF₃ | Ph-4-CF₃ |
| H | CH(CH₃)CH₂OC(O)NHPr-n | CF₃ | Ph-4-OCF₃ |
| H | CH(CH₃)CH₂OC(O)NHPr-n | CF₃ | Ph-4-OCF₂Br |
| H | CH(CH₃)CH₂OC(O)NHPr-n | CF₃ | Ph-4-OCF₂CHFCF₃ |
| H | CH(CH₃)CH₂OC(O)NHPr-n | CF₃ | Ph-4-OCF₂CHFOCF₃ |
| H | CH(CH₃)CH₂OC(O)NHPr-n | CF₃ | Ph-4-OCF₂CHFOCF₂CF₂CF₃ |
| H | CH(CH₃)CH₂OC(O)NHPr-n | CF₃ | Ph-4-O(L-45g) |
| H | CH(CH₃)CH₂OC(O)NHPr-n | CF₃ | Ph-3,4-Cl₂ |
| H | CH(CH₃)CH₂OC(O)NHPr-n | CF₃ | Ph(-3-OCF₂O-4-) |
| H | CH(CH₃)CH₂OC(O)NHPr-n | CF₃ | Ph(-3-OCF₂CF₂O-4-) |
| H | CH(CH₃)CH₂OC(O)NHPr-n | CF₃ | (L-45c)CF₃ |
| H | CH(CH₃)CH₂OC(O)NHPr-n | CF₃ | L-45f |
| H | CH(CH₃)CH₂OC(O)NHPr-n | CF₃ | (L-46c)CF₃ |
| H | CH(CH₃)CH₂OC(O)NHPr-n | CF₃ | L-46d |
| H | CH(CH₃)CH₂OC(O)NHPr-i | CF₃ | CH═CH(Ph-4-Cl) |
| H | CH(CH₃)CH₂OC(O)NHPr-i | CF₃ | Ph-4-Cl |
| H | CH(CH₃)CH₂OC(O)NHPr-i | CF₃ | Ph-4-Br |
| H | CH(CH₃)CH₂OC(O)NHPr-i | CF₃ | Ph-4-CF₃ |
| H | CH(CH₃)CH₂OC(O)NHPr-i | CF₃ | Ph-4-OCF₃ |
| H | CH(CH₃)CH₂OC(O)NHPr-i | CF₃ | Ph-4-OCF₂Br |
| H | CH(CH₃)CH₂OC(O)NHPr-i | CF₃ | Ph-4-OCF₂CHFCF₃ |
| H | CH(CH₃)CH₂OC(O)NHPr-i | CF₃ | Ph-4-OCF₂CHFOCF₃ |
| H | CH(CH₃)CH₂OC(O)NHPr-i | CF₃ | Ph-4-OCF₂CHFOCF₂CF₂CF₂ |
| H | CH(CH₃)CH₂OC(O)NHPr-i | CF₃ | Ph-4-O(L-45g) |
| H | CH(CH₃)CH₂OC(O)NHPr-i | CF₃ | Ph-3,4-Cl₂ |
| H | CH(CH₃)CH₂OC(O)NHPr-i | CF₃ | Ph(-3-OCF₂O-4-) |
| H | CH(CH₃)CH₂OC(O)NHPr-i | CF₃ | Ph(-3-OCF₂CF₂O-4-) |
| H | CH(CH₃)CH₂OC(O)NHPr-i | CF₃ | (L-45c)CF₃ |
| H | CH(CH₃)CH₂OC(O)NHPr-i | CF₃ | L-45f |
| H | CH(CH₃)CH₂OC(O)NHPr-i | CF₃ | (L-46c)CF₃ |
| H | CH(CH₃)CH₂OC(O)NHPr-i | CF₃ | L-46d |
| H | CH(CH₃)CH₂OC(O)NHPr-c | CF₃ | CH═CH(Ph-4-Cl) |
| H | CH(CH₃)CH₂OC(O)NHPr-c | CF₃ | Ph-4-Cl |
| H | CH(CH₃)CH₂OC(O)NHPr-c | CF₃ | Ph-4-Br |
| H | CH(CH₃)CH₂OC(O)NHPr-c | CF₃ | Ph-4-CF₃ |
| H | CH(CH₃)CH₂OC(O)NHPr-c | CF₃ | Ph-4-OCF₃ |
| H | CH(CH₃)CH₂OC(O)NHPr-c | CF₃ | Ph-4-OCF₂Br |
| H | CH(CH₃)CH₂OC(O)NHPr-c | CF₃ | Ph-4-OCF₂CHFCF₃ |
| H | CH(CH₃)CH₂OC(O)NHPr-c | CF₃ | Ph-4-OCF₂CHFOCF₃ |
| H | CH(CH₃)CH₂OC(O)NHPr-c | CF₃ | Ph-4-OCF₂CHFOCF₂CF₂CF₃ |
| H | CH(CH₃)CH₂OC(O)NHPr-c | CF₃ | Ph-4-O(L-45g) |
| H | CH(CH₃)CH₂OC(O)NHPr-c | CF₃ | Ph-3,4-Cl₂ |
| H | CH(CH₃)CH₂OC(O)NHPr-c | CF₃ | Ph(-3-OCF₂O-4-) |
| H | CH(CH₃)CH₂OC(O)NHPr-c | CF₃ | Ph(-3-OCF₂CF₂O-4-) |
| H | CH(CH₃)CH₂OC(O)NHPr-c | CF₃ | (L-45c)CF₃ |
| H | CH(CH₃)CH₂OC(O)NHPr-c | CF₃ | L-45f |
| H | CH(CH₃)CH₂OC(O)NHPr-c | CF₃ | (L-46c)CF₃ |
| H | CH(CH₃)CH₂OC(O)NHPr-c | CF₃ | L-46d |
| H | CH(CH₃)CH₂OC(O)NHBu-t | CF₃ | Ph-4-OCF₃ |
| H | CH(CH₃)CH₃OC(O)NHCH₂CF₃ | CF₃ | (L-45c)CF₃ |
| H | CH(CH₃)CH₂OC(O)NHCH₂CH₂OCH₃ | CF₃ | Ph-4-Br |
| H | CH(CH₃)CH₂OC(O)NHCH₂CH₂SCH₃ | CF₃ | Ph-4-CF₃ |
| H | CH(CH₃)CH₂OC(O)NHCH₂CH₂CH₂SCH₃ | CF₃ | Ph-4-OCF₃ |
| H | CH(CH₃)CH₂OC(O)NHCH₂CH═CH₂ | CF₃ | (L-45c)CF₃ |
| H | CH(CH₃)CH₂OC(O)NHCH₂Ph | CF₃ | CH═CH(Ph-4-Cl) |
| H | CH(CH₃)CH₂OC(O)NHCH₂Ph | CF₃ | Ph-4-Cl |
| H | CH(CH₃)CH₂OC(O)NHCH₂Ph | CF₃ | Ph-4-Br |
| H | CH(CH₃)CH₂OC(O)NHCH₂Ph | CF₃ | Ph-4-CF₃ |
| H | CH(CH₃)CH₂OC(O)NHCH₂Ph | CF₃ | Ph-4-OCF₃ |
| H | CH(CH₃)CH₂OC(O)NHCH₂Ph | CF₃ | Ph-4-OCF₂Br |
| H | CH(CH₃)CH₂OC(O)NHCH₂Ph | CF₃ | Ph-4-OCF₂CHFCF₃ |
| H | CH(CH₃)CH₂OC(O)NHCH₂Ph | CF₃ | Ph-4-OCF₂CHFOCF₃ |
| H | CH(CH₃)CH₂OC(O)NHCH₂Ph | CF₃ | Ph-4-OCF₂CHFOCF₂CF₂CF₃ |
| H | CH(CH₃)CH₂OC(O)NHCH₂Ph | CF₃ | Ph-4-O(L-45g) |
| H | CH(CH₃)CH₂OC(O)NHCH₂Ph | CF₃ | Ph-3,4-Cl₂ |
| H | CH(CH₃)CH₂OC(O)NHCH₂Ph | CF₃ | Ph(-3-OCF₂O-4-) |
| H | CH(CH₃)CH₂OC(O)NHCH₂Ph | CF₃ | Ph(-3-OCF₂CF₂O-4-) |
| H | CH(CH₃)CH₂OC(O)NHCH₂Ph | CF₃ | (L-45c)CF₃ |
| H | CH(CH₃)CH₂OC(O)NHCH₂Ph | CF₃ | L-45f |
| H | CH(CH₃)CH₂OC(O)NHCH₂Ph | CF₃ | (L-46c)CF₃ |
| H | CH(CH₃)CH₂OC(O)NHCH₂Ph | CF₃ | L-46d |
| H | CH(CH₃)CH₂OC(O)NHCH₂(Ph-4-Cl) | CF₃ | Ph-4-Br |
| H | CH(CH₃)CH₂OC(O)NHCH₂(Ph-4-OCH₃) | CF₃ | Ph-4-CF₃ |
| H | CH(CH₃)CH₂OC(O)NHCH₂(L-46a) | CF₃ | Ph-4-OCF₃ |
| H | CH(CH₃)CH₂OC(O)NHCH₂(L-47a) | CF₃ | (L-45c)CF₃ |
| H | CH(CH₃)CH₂OC(O)NHPh | CF₃ | CH═CH(Ph-4-Cl) |
| H | CH(CH₃)CH₂OC(O)NHPh | CF₃ | Ph-4-Cl |
| H | CH(CH₃)CH₂OC(O)NHPh | CF₃ | Ph-4-Br |
| H | CH(CH₃)CH₂OC(O)NHPh | CF₃ | Ph-4-CF₃ |
| H | CH(CH₃)CH₂OC(O)NHPh | CF₃ | Ph-4-OCF₃ |
| H | CH(CH₃)CH₂OC(O)NHPh | CF₃ | Ph-4-OCF₂Br |
| H | CH(CH₃)CH₂OC(O)NHPh | CF₃ | Ph-4-OCF₂CHFCF₃ |
| H | CH(CH₃)CH₂OC(O)NHPh | CF₃ | Ph-4-OCF₂CHFOCF₃ |
| H | CH(CH₃)CH₂OC(O)NHPh | CF₃ | Ph-4-OCF₂CHFOCF₂CF₂CF₃ |
| H | CH(CH₃)CH₂OC(O)NHPh | CF₃ | Ph-4-O(L-45g) |
| H | CH(CH₃)CH₂OC(O)NHPh | CF₃ | Ph-3,4-Cl₂ |
| H | CH(CH₃)CH₂OC(O)NHPh | CF₃ | Ph(-3-OCF₂O-4-) |
| H | CH(CH₃)CH₂OC(O)NHPh | CF₃ | Ph(-3-OCF₂CF₂O-4-) |
| H | CH(CH₃)CH₂OC(O)NHPh | CF₃ | (L-45c)CF₃ |
| H | CH(CH₃)CH₂OC(O)NHPh | CF₃ | L-45f |
| H | CH(CH₃)CH₂OC(O)NHPh | CF₃ | (L-46c)CF₃ |
| H | CH(CH₃)CH₂OC(O)NHPh | CF₃ | L-46d |
| H | CH(CH₃)CH₂OC(O)N(CH₃)₂ | CF₃ | Ph-4-Br |
| H | CH(CH₃)CH₂OC(O)N(Et)₂ | CF₃ | CH═CH(Ph-4-Cl) |
| H | CH(CH₃)CH₂OC(O)N(Et)₂ | CF₃ | Ph-4-Cl |
| H | CH(CH₃)CH₂OC(O)N(Et)₂ | CF₃ | Ph-4-Br |
| H | CH(CH₃)CH₂OC(O)N(Et)₂ | CF₃ | Ph-4-CF₃ |
| H | CH(CH₃)CH₂OC(O)N(Et)₂ | CF₃ | Ph-4-OCF₃ |
| H | CH(CH₃)CH₂OC(O)N(Et)₂ | CF₃ | Ph-4-OCF₂Br |
| H | CH(CH₃)CH₂OC(O)N(Et)₂ | CF₃ | Ph-4-OCF₂CHFCF₃ |
| H | CH(CH₃)CH₂OC(O)N(Et)₂ | CF₃ | Ph-4-OCF₂CHFOCF₃ |
| H | CH(CH₃)CH₂OC(O)N(Et)₂ | CF₃ | Ph-4-OCF₂CHFOCF₂CF₂CF₃ |
| H | CH(CH₃)CH₂OC(O)N(Et)₂ | CF₃ | Ph-4-O(L-45g) |
| H | CH(CH₃)CH₂OC(O)N(Et)₂ | CF₃ | Ph-3,4-Cl₂ |
| H | CH(CH₃)CH₂OC(O)N(Et)₂ | CF₃ | Ph(-3-OCF₂O-4-) |
| H | CH(CH₃)CH₂OC(O)N(Et)₂ | CF₃ | Ph(-3-OCF₂CF₂O-4-) |
| H | CH(CH₃)CH₂OC(O)N(Et)₂ | CF₃ | (L-45c)CF₃ |
| H | CH(CH₃)CH₂OC(O)N(Et)₂ | CF₃ | L-45f |
| H | CH(CH₃)CH₂OC(O)N(Et)₂ | CF₃ | (L-46c)CF₃ |
| H | CH(CH₃)CH₂OC(O)N(Et)₂ | CF₃ | L-46d |
| H | CH(CH₃)CH₂OC(O)N(Pr-i)₂ | CF₃ | Ph-4-CF₃ |
| H | CH(CH₃)CH₂OC(O)N(CH₃)Ph | CF₃ | Ph-4-OCF₃ |
| H | CH(CH₃)CH₂OC(O)(T-16) | CF₃ | (L-45c)CF₃ |
| H | CH(CH₃)CH₂OC(O)(T-19) | CF₃ | Ph-4-Br |
| H | CH(CH₃)CH₂OC(O)(T-20) | CF₃ | Ph-4-CF₃ |
| H | CH(CH₃)CH₂OC(O)(T-21) | CF₃ | Ph-4-OCF₃ |
| H | CH(CH₃)CH₂OP(O)(OEt)₂ | CF₃ | (L-45c)CF₃ |
| H | CH(CH₃)CH₂OP(S)(OCH₃)₂ | CF₃ | CH═CH(Ph-4-Cl) |
| H | CH(CH₃)CH₂OP(S)(OCH₃)₂ | CF₃ | Ph-4-Cl |
| H | CH(CH₃)CH₂OP(S)(OCH₃)₂ | CF₃ | Ph-4-Br |
| H | CH(CH₃)CH₂OP(S)(OCH₃)₂ | CF₃ | Ph-4-CF₃ |
| H | CH(CH₃)CH₂OP(S)(OCH₃)₂ | CF₃ | Ph-4-OCF₃ |
| H | CH(CH₃)CH₂OP(S)(OCH₃)₂ | CF₃ | Ph-4-OCF₂Br |
| H | CH(CH₃)CH₂OP(S)(OCH₃)₂ | CF₃ | Ph-4-OCF₂CHFCF₃ |
| H | CH(CH₃)CH₂OP(S)(OCH₃)₂ | CF₃ | Ph-4-OCF₂CHFOCF₃ |
| H | CH(CH₃)CH₂OP(S)(OCH₃)₂ | CF₃ | Ph-4-OCF₂CHFOCF₂CF₂CF₃ |
| H | CH(CH₃)CH₂OP(S)(OCH₃)₂ | CF₃ | Ph-4-O(L-45g) |
| H | CH(CH₃)CH₂OP(S)(OCH₃)₂ | CF₃ | Ph-3,4-Cl₂ |
| H | CH(CH₃)CH₂OP(S)(OCH₃)₂ | CF₃ | Ph(-3-OCF₂O-4-) |
| H | CH(CH₃)CH₂OP(S)(OCH₃)₂ | CF₃ | Ph(-3-OCF₂CF₂O-4-) |
| H | CH(CH₃)CH₂OP(S)(OCH₃)₂ | CF₃ | (L-45c)CF₃ |
| H | CH(CH₃)CH₂OP(S)(OCH₃)₂ | CF₃ | L-45f |
| H | CH(CH₃)CH₂OP(S)(OCH₃)₂ | CF₃ | (L-46c)CF₃ |
| H | CH(CH₃)CH₂OP(S)(OCH₃)₂ | CF₃ | L-46d |
| H | CH(CH₃)CH₂OP(S)(OEt)₂ | CF₃ | CH═CH(Ph-4-Cl) |
| H | CH(CH₃)CH₂OP(S)(OEt)₂ | CF₃ | Ph-4-Cl |
| H | CH(CH₃)CH₂OP(S)(OEt)₂ | CF₃ | Ph-4-Br |
| H | CH(CH₃)CH₂OP(S)(OEt)₂ | CF₃ | Ph-4-CF₃ |
| H | CH(CH₃)CH₂OP(S)(OEt)₂ | CF₃ | Ph-4-OCF₃ |
| H | CH(CH₃)CH₂OP(S)(OEt)₂ | CF₃ | Ph-4-OCF₂Br |
| H | CH(CH₃)CH₂OP(S)(OEt)₂ | CF₃ | Ph-4-OCF₂CHFCF₃ |
| H | CH(CH₃)CH₂OP(S)(OEt)₂ | CF₃ | Ph-4-OCF₂CHFOCF₃ |
| H | CH(CH₃)CH₂OP(S)(OEt)₂ | CF₃ | Ph-4-OCF₂CHFOCF₂CF₂CF₃ |
| H | CH(CH₃)CH₂OP(S)(OEt)₂ | CF₃ | Ph-4-O(L-45g) |
| H | CH(CH₃)CH₂OP(S)(OEt)₂ | CF₃ | Ph-3,4-Cl₂ |
| H | CH(CH₃)CH₂OP(S)(OEt)₂ | CF₃ | Ph(-3-OCF₂O-4-) |
| H | CH(CH₃)CH₂OP(S)(OEt)₂ | CF₃ | Ph(-3-OCF₂CF_{O-4-)} |
| H | CH(CH₃)CH₂OP(S)(OEt)₂ | CF₃ | (L-45c)CF₃ |
| H | CH(CH₃)CH₂OP(S)(OEt)₂ | CF₃ | L-45f |
| H | CH(CH₃)CH₂OP(S)(OEt)₂ | CF₃ | (L-46c)CF₃ |
| H | CH(CH₃)CH₂OP(S)(OEt)₂ | CF₃ | L-46d |
| H | CH(CH₃)CH₂OPh | CF₃ | Ph-4-Br |
| H | CH(CH₃)CH₂O(Ph-4-Cl) | CF₃ | CH═CH(Ph-4-Cl) |
| H | CH(CH₃)CH₂O(Ph-4-Cl) | CF₃ | Ph-4-Cl |
| H | CH(CH₃)CH₂O(Ph-4-Cl) | CF₃ | Ph-4-Br |
| H | CH(CH₃)CH₂O(Ph-4-Cl) | CF₃ | Ph-4-CF₃ |
| H | CH(CH₃)CH₂O(Ph-4-Cl) | CF₃ | Ph-4-OCF₃ |
| H | CH(CH₃)CH₂O(Ph-4-Cl) | CF₃ | Ph-4-OCF₂Br |
| H | CH(CH₃)CH₂O(Ph-4-Cl) | CF₃ | Ph-4-OCF₂CHFCF₃ |
| H | CH(CH₃)CH₂O(Ph-4-Cl) | CF₃ | Ph-4-OCF₂CHFOCF₃ |
| H | CH(CH₃)CH₂O(Ph-4-Cl) | CF₃ | Ph-4-OCF₂CHFOCF₂CF₂CF₃ |
| H | CH(CH₃)CH₂O(Ph-4-Cl) | CF₃ | Ph-4-O(L-45g) |
| H | CH(CH₃)CH₂O(Ph-4-Cl) | CF₃ | Ph-3,4-Cl₂ |
| H | CH(CH₃)CH₂O(Ph-4-Cl) | CF₃ | Ph(-3-OCF₂O-4-) |
| H | CH(CH₃)CH₂O(Ph-4-Cl) | CF₃ | Ph(-3-OCF₂CF₂O-4-) |
| H | CH(CH₃)CH₂O(Ph-4-Cl) | CF₃ | (L-45c)CF₃ |
| H | CH(CH₃)CH₂O(Ph-4-Cl) | CF₃ | L-45f |
| H | CH(CH₃)CH₂O(Ph-4-Cl) | CF₃ | (L-46c)CF₃ |
| H | CH(CH₃)CH₂O(Ph-4-Cl) | CF₃ | L-46d |
| H | CH(CH₃)CH₂O(Ph-3-CF₃) | CF₃ | Ph-4-CF₃ |
| H | CH(Et)CH₂OH CF₃ | Ph-4-OCF₃ | |
| H | CH(Et)CH₂OCH₃ | CF₃ | (L-45c)CF₃ |
| H | CH(Ph)CH₂OH | CF₃ | Ph-4-Br |
| H | CH(Ph)CH₂OH(R) | CF₃ | Ph-4-Br |
| H | CH(Ph-2-Cl)CH₂OH | CF₃ | Ph-4-CF₃ |
| H | CH(Ph-4-Cl)CH₂OH | CF₃ | Ph-4-OCF₃ |
| H | CH(Ph-4-Ph)CH₂OH | CF₃ | (L-45c)CF₃ |
| H | C(CH₃)₂CH₂OH | CF₃ | Ph-4-Br |
| H | CH(CH₃)CH₂OCH₃ | CH₃ | Ph-4-Br |
| H | C(CH₃)₂CH₂OCH₃ | CH₃ | Ph-4-CF₃ |
| H | C(CH₃)₂CH₂OCH₃ | CH₃ | Ph-4-OCF₃ |
| H | C(CH₃)₂CH₂OCH₃ | CH₃ | (L-45c)Cl |
| H | C(CH₃)₂CH₂OCH₃ | CH₃ | (L-45c)Br |
| H | C(CH₃)₂CH₂OCH₃ | CH₃ | (L-45c)CF₃ |
| H | C(CH₃)₂CH₂OCH₃ | Et | Ph-4-CF₃ |
| H | C(CH₃)₂CH₂OCH₃ | n-Pr | Ph-4-OCF₃ |
| H | C(CH₃)₂CH₂OCH₃ | i-Pr | (L-45c)CF₃ |
| H | C(CH₃)₂CH₂OCH₃ | c-Pr | Ph-4-Br |
| H | C(CH₃)₂CH₂OCH₃ | CHF₂ | Ph-4-CF₃ |
| H | C(CH₃)₂CH₂OCH₃ | CF₃ | CF₂O(Ph-4-Cl) |
| H | C(CH₃)₂CH₂OCH₃ | CF₃ | CH═CH(Ph-4-Cl) |
| H | C(CH₃)₂CH₂OCH₃ | CF₃ | CH═CH(Ph-4-OCF₃) |
| H | C(CH₃)₂CH₂OCH₃ | CF₃ | CH═CH(Ph-3,4-Cl₂) |
| H | C(CH₃)₂CH₂OCH₃ | CF₃ | Ph-4-F |
| H | C(CH₃)₂CH₂OCH₃ | CF₃ | Ph-4-Cl |
| H | C(CH₃)₂CH₂OCH₃ | CF₃ | Ph-4-Br |
| H | C(CH₃)₂CH₂OCH₃ | CF₃ | Ph-4-I |
| H | C(CH₃)₂CH₂OCH₃ | CF₃ | Ph-4-CF₃ |
| H | C(CH₃)₂CH₂OCH₃ | CF₃ | Ph-4-OCHF₂ |
| H | C(CH₃)₂CH₂OCH₃ | CF₃ | Ph-4-OCF₃ |
| H | C(CH₃)₂CH₂OCH₃ | CF₃ | Ph-4-OCF₂Br |
| H | C(CH₃)₂CH₂OCH₃ | CF₃ | Ph-4-OCF₂CHF₂ |
| H | C(CH₃)₂CH₂OCH₃ | CF₃ | Ph-4-OCF₂CHFCl |
| H | C(CH₃)₂CH₂OCH₃ | CF₃ | Ph-4-OCF₂CHFCF₃ |
| H | C(CH₃)₂CH₂OCH₃ | CF₃ | Ph-4-OCF₂CHFOCF₃ |
| H | C(CH₃)₂CH₂OCH₃ | CF₃ | Ph-4-OCF₂CHFOCF₂CF₂CF₃ |
| H | C(CH₃)₂CH₂OCH₃ | CF₃ | Ph-4-O(L-45g) |
| H | C(CH₃)₂CH₂OCH₃ | CF₃ | Ph-3,4-Cl₂ |
| H | C(CH₃)₂CH₂OCH₃ | CF₃ | Ph(-3-OCF₂O-4-) |
| H | C(CH₃)₂CH₂OCH₃ | CF₃ | Ph(-3-OCf₂CF₂O-4-) |
| H | C(CH₃)₂CH₂OCH₃ | CF₃ | (L-45c)Cl |
| H | C(CH₃)₂CH₂OCH₃ | CF₃ | (L-45c)Br |
| H | C(CH₃)₂CH₂OCH₃ | CF₃ | (L-45c)CF₃ |
| H | C(CH₃)₂CH₂OCH₃ | CF₃ | L-45e |
| H | C(CH₃)₂CH₂OCH₃ | CF₃ | L-45f |
| H | C(CH₃)₂CH₂OCH₃ | CF₃ | (L-46c)Cl |
| H | C(CH₃)₂CH₂OCH₃ | CF₃ | (L-46c)Br |
| H | C(CH₃)₂CH₂OCH₃ | CF₃ | (L-46c)CF₃ |
| H | C(CH₃)₂CH₂OCH₃ | CF₃ | L-46d |
| H | C(CH₃)₂CH₂OCH₃ | CF₂Cl | Ph-4-OCF₃ |
| H | C(CH₃)₂CH₂OCH₃ | CF₂Br | (L-45c)CF₃ |
| H | C(CH₃)₂CH₂OCH₃ | CF₂CF₃ | Ph-4-Br |
| H | C(CH₃)₂CH₂OCH₃ | CF₂CF₂CF₃ | Ph-4-CF₃ |
| H | C(CH₃)₂CH₂OCH₃ | CF₂OCH₃ | Ph-4-OCF₃ |
| H | C(CH₃)₂CH₂OCH₃ | CF₂SCH₃ | (L-45c)CF₃ |
| H | C(CH₃)₂CH₂OC(O)CH₃ | CF₃ | Ph-4-Br |
| H | C(CH₃)₂CH₂OC(O)CF₃ | CF₃ | Ph-4-CF₃ |
| H | C(CH₃)₂CH₂OC(O)NHCH₃ | CH₃ | Ph-4-Br |
| H | C(CH₃)₂CH₂OC(O)NHCH₃ | CH₃ | Ph-4-CF₃ |
| H | C(CH₃)₂CH₂OC(O)NHCH₃ | CH₃ | Ph-4-OCF₃ |
| H | C(CH₃)₂CH₂OC(O)NHCH₃ | CH₃ | (L-45c)Cl |
| H | C(CH₃)₂CH₂OC(O)NHCH₃ | CH₃ | (L-45c)Br |
| H | C(CH₃)₂CH₂OC(O)NHCH₃ | CH₃ | (L-45c)CF₃ |
| H | C(CH₃)₂CH₂OC(O)NHCH₃ | Et | Ph-4-OCF₃ |
| H | C(CH₃)₂CH₂OC(O)NHCH₃ | n-Pr | (L-45c)CF₃ |
| H | C(CH₃)₂CH₂OC(O)NHCH₃ | i-Pr | Ph-4-Br |
| H | C(CH₃)₂CH₂OC(O)NHCH₃ | c-Pr | Ph-4-CF₃ |
| H | C(CH₃)₂CH₂OC(O)NHCH₃ | CHF₂ | Ph-4-OCF₃ |
| H | C(CH₃)₂CH₂OC(O)NHCH₃ | CF₃ | CF₂O(Ph-4-Cl) |
| H | C(CH₃)₂CH₂OC(O)NHCH₃ | CF₃ | CH═CH(Ph-4-Cl) |
| H | C(CH₃)₂CH₂OC(O)NHCH₃ | CF₃ | CH═CH(Ph-4-Br) |
| H | C(CH₃)₂CH₂OC(O)NHCH₃ | CF₃ | CH═CH(Ph-4-CF₃) |
| H | C(CH₃)₂CH₂OC(O)NHCH₃ | CF₃ | CH═CH(Ph-4-OCF₃) |
| H | C(CH₃)₂CH₂OC(O)NHCH₃ | CF₃ | CH═CH(Ph-3,4-Cl₂) |
| H | C(CH₃)₂CH₂OC(O)NHCH₃ | CF₃ | Ph-4-F |
| H | C(CH₃)₂CH₂OC(O)NHCH₃ | CF₃ | Ph-4-Cl |
| H | C(CH₃)₂CH₂OC(O)NHCH₃ | CF₃ | Ph-4-Br |
| H | C(CH₃)₂CH₂OC(O)NHCH₃ | CF₃ | Ph-4-I |
| H | C(CH₃)₂CH₂OC(O)NHCH₃ | CF₃ | Ph-4-CF₃ |
| H | C(CH₃)₂CH₂OC(O)NHCH₃ | CF₃ | Ph-4-OCHF₂ |
| H | C(CH₃)₂CH₂OC(O)NHCH₃ | CF₃ | Ph-4-OCF₃ |
| H | C(CH₃)₂CH₂OC(O)NHCH₃ | CF₃ | Ph-4-OCF₂Br |
| H | C(CH₃)₂CH₂OC(O)NHCH₃ | CF₃ | Ph-4-OCF₂CHF₂ |
| H | C(CH₃)₂CH₂OC(O)NHCH₃ | CF₃ | Ph-4-OCF₂CHFCl |
| H | C(CH₃)₂CH₂OC(O)NHCH₃ | CF₃ | Ph-4-OCF₂CHFCF₃ |
| H | C(CH₃)₂CH₂OC(O)NHCH₃ | CF₃ | Ph-4-OCF₂CHFOCF₃ |
| H | C(CH₃)₂CH₂OC(O)NHCH₃ | CF₃ | Ph-4-OCF₂CHFOCF₂CF₂CF₃ |
| H | C(CH₃)₂CH₂OC(O)NHCH₃ | CF₃ | Ph-4-O(L-45g) |
| H | C(CH₃)₂CH₂OC(O)NHCH₃ | CF₃ | Ph-3,4-F₂ |
| H | C(CH₃)₂CH₂OC(O)NHCH₃ | CF₃ | Ph-3-F-4-Cl |
| H | C(CH₃)₂CH₂OC(O)NHCH₃ | CF₃ | Ph-3,4-Cl₂ |
| H | C(CH₃)₂CH₂OC(O)NHCH₃ | CF₃ | Ph(-3-OCF₂O-4-) |
| H | C(CH₃)₂CH₂OC(O)NHCH₃ | CF₃ | Ph(-3-OCF₂CF₂O-4-) |
| H | C(CH₃)₂CH₂OC(O)NHCH₃ | CF₃ | (L-45c)Cl |
| H | C(CH₃)₂CH₂OC(O)NHCH₃ | CF₃ | (L-45c)Br |
| H | C(CH₃)₂CH₂OC(O)NHCH₃ | CF₃ | (L-45c)CF₃ |
| H | C(CH₃)₂CH₂OC(O)NHCH₃ | CF₃ | L-45e |
| H | C(CH₃)₂CH₂OC(O)NHCH₃ | CF₃ | L-45f |
| H | C(CH₃)₂CH₂OC(O)NHCH₃ | CF₃ | (L-46c)Cl |
| H | C(CH₃)₂CH₂OC(O)NHCH₃ | CF₃ | (L-46c)Br |
| H | C(CH₃)₂CH₂OC(O)NHCH₃ | CF₃ | (L-46c)CF₃ |
| H | C(CH₃)₂CH₂OC(O)NHCH₃ | CF₃ | L-46d |
| H | C(CH₃)₂CH₂OC(O)NHCH₃ | CF₂Cl | (L-45c)CF₃ |
| H | C(CH₃)₂CH₂OC(O)NHCH₃ | CF₂Br | Ph-4-Br |
| H | C(CH₃)₂CH₂OC(O)NHCH₃ | CF₂CF₃ | Ph-4-CF₃ |
| H | C(CH₃)₂CH₂OC(O)NHCH₃ | CF₂CF₂CF₃ | Ph-4-OCF₃ |
| H | C(CH₃)₂CH₂OC(O)NHCH₃ | CF₂OCH₃ | (L-45c)CF₃ |
| H | C(CH₃)₂CH₂OC(O)NHCH₃ | CF₂SCH₃ | Ph-4-Br |
| H | C(CH₃)₂CH₂OC(O)NHEt | CF₃ | CF₂O(Ph-4-Cl) |
| H | C(CH₃)₂CH₂OC(O)NHEt | CF₃ | CH═CH(Ph-4-Cl) |
| H | C(CH₃)₂CH₂OC(O)NHEt | CF₃ | CH═CH(Ph-4-Br) |
| H | C(CH₃)₂CH₂OC(O)NHEt | CF₃ | CH═CH(Ph-4-CF₃) |
| H | C(CH₃)₂CH₂OC(O)NHEt | CF₃ | CH═CH(Ph-4-OCF₃) |
| H | C(CH₃)₂CH₂OC(O)NHEt | CF₃ | CH═CH(Ph-3,4-Cl₂) |
| H | C(CH₃)₂CH₂OC(O)NHEt | CF₃ | Ph-4-F |
| H | C(CH₃)₂CH₂OC(O)NHEt | CF₃ | Ph-4-Cl |
| H | C(CH₃)₂CH₂OC(O)NHEt | CF₃ | Ph-4-Br |
| H | C(CH₃)₂CH₂OC(O)NHEt | CF₃ | Ph-4-CF₃ |
| H | C(CH₃)₂CH₂OC(O)NHEt | CF₃ | Ph-4-OCF₃ |
| H | C(CH₃)₂CH₂OC(O)NHEt | CF₃ | Ph-4-OCF₂Br |
| H | C(CH₃)₂CH₂OC(O)NHEt | CF₃ | Ph-4-OCF₂CHF₂ |
| H | C(CH₃)₂CH₂OC(O)NHEt | CF₃ | Ph-4-OCF₂CHFCF₃ |
| H | C(CH₃)₂CH₂OC(O)NHEt | CF₃ | Ph-4-OCF₂CHFOCF₃ |
| H | C(CH₃)₂CH₂OC(O)NHEt | CF₃ | Ph-4-OCF₂CHFOCF₂CF₂CF₃ |
| H | C(CH₃)₂CH₂OC(O)NHEt | CF₃ | Ph-4-O(L-45g) |
| H | C(CH₃)₂CH₂OC(O)NHEt | CF₃ | Ph-3,4-F₂ |
| H | C(CH₃)₂CH₂OC(O)NHEt | CF₃ | Ph-3-F-4-Cl |
| H | C(CH₃)₂CH₂OC(O)NHEt | CF₃ | Ph-3,4-Cl₂ |
| H | C(CH₃)₂CH₂OC(O)NHEt | CF₃ | Ph(-3-OCF₂O-4-) |
| H | C(CH₃)₂CH₂OC(O)NHEt | CF₃ | Ph(-3-OCF₂CF₂O-4-) |
| H | C(CH₃)₂CH₂OC(O)NHEt | CF₃ | (L-45c)CF₃ |
| H | C(CH₃)₂CH₂OC(O)NHEt | CF₃ | L-45f |
| H | C(CH₃)₂CH₂OC(O)NHEt | CF₃ | (L-46c)CF₃ |
| H | C(CH₃)₂CH₂OC(O)NHEt | CF₃ | L-46d |
| H | C(CH₃)₂CH₂OC(O)NHPr-n | CF₃ | CH═CH(Ph-4-Cl) |
| H | C(CH₃)₂CH₂OC(O)NHPr-n | CF₃ | Ph-4-Cl |
| H | C(CH₃)₂CH₂OC(O)NHPr-n | CF₃ | Ph-4-Br |
| H | C(CH₃)₂CH₂OC(O)NHPr-n | CF₃ | Ph-4-CF₃ |
| H | C(CH₃)₂CH₂OC(O)NHPr-n | CF₃ | Ph-4-OCF₃ |
| H | C(CH₃)₂CH₂OC(O)NHPr-n | CF₃ | Ph-4-OCF₂Br |
| H | C(CH₃)₂CH₂OC(O)NHPr-n | CF₃ | Ph-4-OCF₂CHFCF₃ |
| H | C(CH₃)₂CH₂OC(O)NHPr-n | CF₃ | Ph-4-OCF₂CHFOCF₃ |
| H | C(CH₃)₂CH₂OC(O)NHPr-n | CF₃ | Ph-4-OCF₂CHFOCF₂CF₂CF₃ |
| H | C(CH₃)₂CH₂OC(O)NHPr-n | CF₃ | Ph-4-O(L-45g) |
| H | C(CH₃)₂CH₂OC(O)NHPr-n | CF₃ | Ph-3,4-Cl₂ |
| H | C(CH₃)₂CH₂OC(O)NHPr-n | CF₃ | Ph(-3-OCF₂O-4-) |
| H | C(CH₃)₂CH₂OC(O)NHPr-n | CF₃ | Ph(-3-OCF₂CF₂O-4-) |
| H | C(CH₃)₂CH₂OC(O)NHPr-n | CF₃ | (L-45c)CF₃ |
| H | C(CH₃)₂CH₂OC(O)NHPr-n | CF₃ | L-45f |
| H | C(CH₃)₂CH₂OC(O)NHPr-n | CF₃ | (L-46c)CF₃ |
| H | C(CH₃)₂CH₂OC(O)NHPr-n | CF₃ | L-46d |
| H | C(CH₃)₂CH₂OC(O)NHPr-i | CF₃ | Ph-4-CF₃ |
| H | C(CH₃)₂CH₂OC(O)NHPr-c | CF₃ | Ph-4-OCF₃ |
| H | C(CH₃)₂CH₂OC(O)NHCH₂CF₃ | CF₃ | (L-45c)CF₃ |
| H | C(CH₃)₂CH₂OC(O)NHCH₂CH₂OCH₃ | CF₃ | Ph-4-Br |
| H | C(CH₃)₂CH₂OC(O)NHCH₂CH₂SCH₃ | CF₃ | Ph-4-CF₃ |
| H | C(CH₃)₂CH₂OC(O)NHCH₂CH₂CH₂SCH₃ | CF₃ | Ph-4-OCF₃ |
| H | C(CH₃)₂CH₂OC(O)NHCH₂CH═CH₂ | CF₃ | Ph-4-CF₃ |
| H | C(CH₃)₂CH₂OC(O)NHCH₂Ph | CF₃ | CH═CH(Ph-4-Cl) |
| H | C(CH₃)₂CH₂OC(O)NHCH₂Ph | CF₃ | Ph-4-Cl |
| H | C(CH₃)₂CH₂OC(O)NHCH₂Ph | CF₃ | Ph-4-Br |
| H | C(CH₃)₂CH₂OC(O)NHCH₂Ph | CF₃ | Ph-4-CF₃ |
| H | C(CH₃)₂CH₂OC(O)NHCH₂Ph | CF₃ | Ph-4-OCF₃ |
| H | C(CH₃)₂CH₂OC(O)NHCH₂Ph | CF₃ | Ph-4-OCF₂Br |
| H | C(CH₃)₂CH₂OC(O)NHCH₂Ph | CF₃ | Ph-4-OCF₂CHFCF₃ |
| H | C(CH₃)₂CH₂OC(O)NHCH₂Ph | CF₃ | Ph-4-OCF₂CHFOCF₃ |
| H | C(CH₃)₂CH₂OC(O)NHCH₂Ph | CF₃ | Ph-4-OCF₂CHFOCF₂CF₂CF₃ |
| H | C(CH₃)₂CH₂OC(O)NHCH₂Ph | CF₃ | Ph-4-O(L-45g) |
| H | C(CH₃)₂CH₂OC(O)NHCH₂Ph | CF₃ | Ph-3,4-Cl₂ |
| H | C(CH₃)₂CH₂OC(O)NHCH₂Ph | CF₃ | Ph(-3-OCF₂O-4-) |
| H | C(CH₃)₂CH₂OC(O)NHCH₂Ph | CF₃ | Ph(-3-OCF₂CF₂O-4-) |
| H | C(CH₃)₂CH₂OC(O)NHCH₂Ph | CF₃ | (L-45c)CF₃ |
| H | C(CH₃)₂CH₂OC(O)NHCH₂Ph | CF₃ | L-45f |
| H | C(CH₃)₂CH₂OC(O)NHCH₂Ph | CF₃ | (L-46c)CF₃ |
| H | C(CH₃)₂CH₂OC(O)NHCH₂Ph | CF₃ | L-46d |
| H | C(CH₃)₂CH₂OC(O)N(CH₃)₂ | CF₃ | CH═CH(Ph-4-Cl) |
| H | C(CH₃)₂CH₂OC(O)N(CH₃)₂ | CF₃ | Ph-4-Cl |
| H | C(CH₃)₂CH₂OC(O)N(CH₃)₂ | CF₃ | Ph-4-Br |
| H | C(CH₃)₂CH₂OC(O)N(CH₃)₂ | CF₃ | Ph-4-CF₃ |
| H | C(CH₃)₂CH₂OC(O)N(CH₃)₂ | CF₃ | Ph-4-OCF₃ |
| H | C(CH₃)₂CH₂OC(O)N(CH₃)₂ | CF₃ | Ph-4-OCF₂Br |
| H | C(CH₃)₂CH₂OC(O)N(CH₃)₂ | CF₃ | Ph-4-OCF₂CHFCF₃ |
| H | C(CH₃)₂CH₂OC(O)N(CH₃)₂ | CF₃ | Ph-4-OCF₂CHFOCF₃ |
| H | C(CH₃)₂CH₂OC(O)N(CH₃)₂ | CF₃ | Ph-4-OCF₂CHFOCF₂CF₂CF₃ |
| H | C(CH₃)₂CH₂OC(O)N(CH₃)₂ | CF₃ | Ph-4-O(L-45g) |
| H | C(CH₃)₂CH₂OC(O)N(CH₃)₂ | CF₃ | Ph-3,4-Cl₂ |
| H | C(CH₃)₂CH₂OC(O)N(CH₃)₂ | CF₃ | Ph(-3-OCF₂O-4-) |
| H | C(CH₃)₂CH₂OC(O)N(CH₃)₂ | CF₃ | Ph(-3-OCF₂CF₂O-4-) |
| H | C(CH₃)₂CH₂OC(O)N(CH₃)₂ | CF₃ | (L-45c)CF₃ |
| H | C(CH₃)₂CH₂OC(O)N(CH₃)₂ | CF₃ | L-45f |
| H | C(CH₃)₂CH₂OC(O)N(CH₃)₂ | CF₃ | (L-46c)CF₃ |
| H | C(CH₃)₂CH₂OC(O)N(CH₃)₂ | CF₃ | L-46d |
| H | C(CH₃)₂CH₂OC(O)N(CH₃)₂ | CF₃ | Ph-4-OCF₃ |
| H | CH₂CH₂CH₂OH | CF₃ | (L-45c)CF₃ |
| H | CH₂CH₂CH₂OCH₃ | CF₃ | Ph-4-Br |
| H | CH(CH₃)CH₂CH₂OCH₃ | CF₃ | Ph-4-CF₃ |
| H | CH(CH₃)CH₂CH₂OEt | CF₃ | CH═CH(Ph-4-Cl) |
| H | CH(CH₃)CH₂CH₂OEt | CF₃ | Ph-4-Cl |
| H | CH(CH₃)CH₂CH₂OEt | CF₃ | Ph-4-Br |
| H | CH(CH₃)CH₂CH₂OEt | CF₃ | Ph-4-CF₃ |
| H | CH(CH₃)CH₂CH₂OEt | CF₃ | Ph-4-OCF₃ |
| H | CH(CH₃)CH₂CH₂OEt | CF₃ | Ph-4-OCF₂Br |
| H | CH(CH₃)CH₂CH₂OEt | CF₃ | Ph-4-OCF₂CHFCF₃ |
| H | CH(CH₃)CH₂CH₂OEt | CF₃ | Ph-4-OCF₂CHFOCF₃ |
| H | CH(CH₃)CH₂CH₂OEt | CF₃ | Ph-4-OCF₂CHFOCF₂CF₂CF₃ |
| H | CH(CH₃)CH₂CH₂OEt | CF₃ | Ph-4-O(L-45g) |
| H | CH(CH₃)CH₂CH₂OEt | CF₃ | Ph-3,4-Cl₂ |
| H | CH(CH₃)CH₂CH₂OEt | CF₃ | Ph(-3-OCF₂O-4-) |
| H | CH(CH₃)CH₂CH₂OEt | CF₃ | Ph(-3-OCF₂CF₂O-4-) |
| H | CH(CH₃)CH₂CH₂OEt | CF₃ | (L-45c)CF₃ |
| H | CH(CH₃)CH₂CH₂OEt | CF₃ | L-45f |
| H | CH(CH₃)CH₂CH₂OEt | CF₃ | (L-46c)CF₃ |
| H | CH(CH₃)CH₂CH₂OEt | CF₃ | L-46d |
| H | CH(CH₃)CH₂CH₂OPr-n | CF₃ | Ph-4-OCF₃ |
| H | CH(CH₃)CH₂CH₂OBu-i | CF₃ | (L-45c)CF₃ |
| H | CH(CH₃)CH₂CH₂OCH₂CF₃ | CF₃ | Ph-4-Br |
| H | CH(CH₃)CH₂CH₂OCH₂CH₂OCH₃ | CF₃ | Ph-4-CF₃ |
| H | CH(CH₃)CH₂CH₂OC(O)NHEt | CF₃ | Ph-4-OCF₃ |
| H | CH₂CH₂CH₂CH₂OC(O)NHEt | CF₃ | (L-45c)CF₃ |
| H | CH₂CH₂CH₂CH₂OC(O)NHPr-i | CF₃ | Ph-4-Br |
| H | CH₂CH₂CH₂CH₂CH₂OC(O)NHEt | CF₃ | Ph-4-CF₃ |
| H | T-10 | CF₃ | Ph-4-OCF₃ |
| H | M-4a | CF₃ | (L-45c)CF₃ |
| H | M-5a | CF₃ | Ph-4-Br |
| H | CH₂(M-7a) | CF₃ | Ph-4-CF₃ |
| H | CH₂(M-16a) | CF₃ | Ph-4-OCF₃ |
| —CH₂CH₂OCH₂CH₂— | CF₃ | (L-45c)CF₃ | |
| H | CH₂SCH₃ | CF₃ | Ph-4-Br |
| H | CH₂CH₂SCH₃ | CF₃ | Ph-4-CF₃ |
| H | CH₂CH₂SEt | CF₃ | Ph-4-OCF₃ |
| H | CH₂CH₂SPr-i | CF₃ | (L-45c)CF₃ |
| H | CH₂CH(CH₃)SCH₃ | CF₃ | Ph-4-Br |
| H | CH₂CH(CH₃)SO₂CH₃ | CF₃ | Ph-4-CF₃ |
| H | CH₂CH(CH₃)SEt | CF₃ | Ph-4-OCF₃ |
| H | CH₂CH(CH₃)SO₂Et | CF₃ | (L-45c)CF₃ |
| H | CH(CH₃)CH₂SH | CF₃ | Ph-4-Br |
| H | CH(CH₃)CH₂SCH₃ | CH₃ | CF₂O(Ph-4-Cl) |
| H | CH(CH₃)CH₂SCH₃ | CH₃ | CH═CH(Ph-4-Cl) |
| H | CH(CH₃)CH₂SCH₃ | CH₃ | CH═CH(Ph-4-OCF₃) |
| H | CH(CH₃)CH₂SCH₃ | CH₃ | CH═CH(Ph-3,4-Cl₂) |
| H | CH(CH₃)CH₂SCH₃ | CH₃ | Ph-4-F |
| H | CH(CH₃)CH₂SCH₃ | CH₃ | Ph-4-Cl |
| H | CH(CH₃)CH₂SCH₃ | CH₃ | Ph-4-Br |
| H | CH(CH₃)CH₂SCH₃ | CH₃ | Ph-4-I |
| H | CH(CH₃)CH₂SCH₃ | CH₃ | Ph-4-CF₃ |
| H | CH(CH₃)CH₂SCH₃ | CH₃ | Ph-4-OCHF₂ |
| H | CH(CH₃)CH₂SCH₃ | CH₃ | Ph-4-OCF₃ |
| H | CH(CH₃)CH₂SCH₃ | CH₃ | Ph-4-OCF₂Br |
| H | CH(CH₃)CH₂SCH₃ | CH₃ | Ph-4-OCF₂CHF₂ |
| H | CH(CH₃)CH₂SCH₃ | CH₃ | Ph-4-OCF₂CHFCl |
| H | CH(CH₃)CH₂SCH₃ | CH₃ | Ph-4-OCF₂CHFCF₃ |
| H | CH(CH₃)CH₂SCH₃ | CH₃ | Ph-4-OCF₂CHFOCF₃ |
| H | CH(CH₃)CH₂SCH₃ | CH₃ | Ph-4-OCF₂CHFOCF₂CF₂CF₃ |
| H | CH(CH₃)CH₂SCH₃ | CH₃ | Ph-4-O(L-45g) |
| H | CH(CH₃)CH₂SCH₃ | CH₃ | Ph-3,4-Cl₂ |
| H | CH(CH₃)CH₂SCH₃ | CH₃ | Ph(-3-OCF₂O-4-) |
| H | CH(CH₃)CH₂SCH₃ | CH₃ | Ph(-3-OCF₂CF₂O-4-) |
| H | CH(CH₃)CH₂SCH₃ | CH₃ | (L-45c)Cl |
| H | CH(CH₃)CH₂SCH₃ | CH₃ | (L-45c)Br |
| H | CH(CH₃)CH₂SCH₃ | CH₃ | (L-45c)CF₃ |
| H | CH(CH₃)CH₂SCH₃ | CH₃ | L-45e |
| H | CH(CH₃)CH₂SCH₃ | CH₃ | L-45f |
| H | CH(CH₃)CH₂SCH₃ | CH₃ | (L-46c)Cl |
| H | CH(CH₃)CH₂SCH₃ | CH₃ | (L-46c)Br |
| H | CH(CH₃)CH₂SCH₃ | CH₃ | (L-46c)CF₃ |
| H | CH(CH₃)CH₂SCH₃ | CH₃ | L-46d |
| H | CH(CH₃)CH₂SCH₃ | Et | Ph-4-Cl |
| H | CH(CH₃)CH₂SCH₃ | Et | Ph-4-Br |
| H | CH(CH₃)CH₂SCH₃ | Et | Ph-4-CF₃ |
| H | CH(CH₃)CH₂SCH₃ | Et | Ph-4-OCF₃ |
| H | CH(CH₃)CH₂SCH₃ | Et | Ph-4-OCF₂Br |
| H | CH(CH₃)CH₂SCH₃ | Et | Ph-4-OCF₂CHFCF₃ |
| H | CH(CH₃)CH₂SCH₃ | Et | Ph-4-OCF₂CHFOCF₃ |
| H | CH(CH₃)CH₂SCH₃ | Et | Ph-4-OCF₂CHFOCF₂CF₂CF₃ |
| H | CH(CH₃)CH₂SCH₃ | Et | Ph-4-O(L-45g) |
| H | CH(CH₃)CH₂SCH₃ | Et | Ph-3,4-Cl₂ |
| H | CH(CH₃)CH₂SCH₃ | Et | Ph(-3-OCF₂O-4-) |
| H | CH(CH₃)CH₂SCH₃ | Et | Ph(-3-OCF₂CF₂O-4-) |
| H | CH(CH₃)CH₂SCH₃ | Et | (L-45c)CF₃ |
| H | CH(CH₃)CH₂SCH₃ | n-Pr | Ph-4-Br |
| H | CH(CH₃)CH₂SCH₃ | n-Pr | Ph-4-CF₃ |
| H | CH(CH₃)CH₂SCH₃ | n-Pr | Ph-4-OCF₃ |
| H | CH(CH₃)CH₂SCH₃ | n-Pr | (L-45c)CF₃ |
| H | CH(CH₃)CH₂SCH₃ | i-Pr | Ph-4-Br |
| H | CH(CH₃)CH₂SCH₃ | i-Pr | Ph-4-CF₃ |
| H | CH(CH₃)CH₂SCH₃ | i-Pr | Ph-4-OCF₃ |
| H | CH(CH₃)CH₂SCH₃ | i-Pr | (L-45c)CF₃ |
| H | CH(CH₃)CH₂SCH₃ | c-Pr | Ph-4-Br |
| H | CH(CH₃)CH₂SCH₃ | c-Pr | Ph-4-CF₃ |
| H | CH(CH₃)CH₂SCH₃ | c-Pr | Ph-4-OCF₃ |
| H | CH(CH₃)CH₂SCH₃ | c-Pr | (L-45c)CF₃ |
| H | CH(CH₃)CH₂SCH₃ | n-Bu | Ph-4-CF₃ |
| H | CH(CH₃)CH₂SCH₃ | CH₂F | Ph-4-OCF₃ |
| H | CH(CH₃)CH₂SCH₃ | CH₂Cl | (L-45c)CF₃ |
| H | CH(CH₃)CH₂SCH₃ | CH₂Br | Ph-4-Br |
| H | CH(CH₃)CH₂SCH₃ | CHF₂ | Ph-4-Cl |
| H | CH(CH₃)CH₂SCH₃ | CHF₂ | Ph-4-Br |
| H | CH(CH₃)CH₂SCH₃ | CHF₂ | Ph-4-CF₃ |
| H | CH(CH₃)CH₂SCH₃ | CHF₂ | Ph-4-OCF₃ |
| H | CH(CH₃)CH₂SCH₃ | CHF₂ | Ph-4-OCF₂Br |
| H | CH(CH₃)CH₂SCH₃ | CHF₂ | Ph-4-OCF₂CHFCF₃ |
| H | CH(CH₃)CH₂SCH₃ | CHF₂ | Ph-4-OCF₂CHFOCF₃ |
| H | CH(CH₃)CH₂SCH₃ | CHF₂ | Ph-4-OCF₂CHFOCF₂CF₂CF₃ |
| H | CH(CH₃)CH₂SCH₃ | CHF₂ | Ph-4-O(L-45g) |
| H | CH(CH₃)CH₂SCH₃ | CHF₂ | Ph-3,4-Cl₂ |
| H | CH(CH₃)CH₂SCH₃ | CHF₂ | Ph(-3-OCF₂O-4-) |
| H | CH(CH₃)CH₂SCH₃ | CHF₂ | Ph(-3-OCF₂CF_{O-4-)} |
| H | CH(CH₃)CH₂SCH₃ | CHF₂ | (L-45c)CF₃ |
| H | CH(CH₃)CH₂SCH₃ | CHF₂ | L-45f |
| H | CH(CH₃)CH₂SCH₃ | CHF₂ | (L-46c)CF₃ |
| H | CH(CH₃)CH₂SCH₃ | CHF₂ | L-46d |
| H | CH(CH₃)CH₂SCH₃ | CHFCl | Ph-4-CF₃ |
| H | CH(CH₃)CH₂SCH₃ | CHFBr | Ph-4-OCF₃ |
| H | CH(CH₃)CH₂SCH₃ | CF₃ | c-Pr |
| H | CH(CH₃)CH₂SCH₃ | CF₃ | c-Bu |
| H | CH(CH₃)CH₂SCH₃ | CF₃ | c-Pen |
| H | CH(CH₃)CH₂SCH₃ | CF₃ | c-Hex |
| H | CH(CH₃)CH₂SCH₃ | CF₃ | T-1 |
| H | CH(CH₃)CH₂SCH₃ | CF₃ | T-2 |
| H | CH(CH₃)CH₂SCH₃ | CF₃ | T-3 |
| H | CH(CH₃)CH₂SCH₃ | CF₃ | T-4 |
| H | CH(CH₃)CH₂SCH₃ | CF₃ | T-5 |
| H | CH(CH₃)CH₂SCH₃ | CF₃ | CH₂OCH₃ |
| H | CH(CH₃)CH₂SCH₃ | CF₃ | CH₂OEt |
| H | CH(CH₃)CH₂SCH₃ | CF₃ | CH₂OPr-n |
| H | CH(CH₃)CH₂SCH₃ | CF₃ | CH₂OPr-i |
| H | CH(CH₃)CH₂SCH₃ | CF₃ | CH₂OBu-n |
| H | CH(CH₃)CH₂SCH₃ | CF₃ | CH₂OC(O)(Ph-2-Cl) |
| H | CH(CH₃)CH₂SCH₃ | CF₃ | CH₂OC(O)(Ph-3-Cl) |
| H | CH(CH₃)CH₂SCH₃ | CF₃ | CH₂OC(O)(Ph-4-Cl) |
| H | CH(CH₃)CH₂SCH₃ | CF₃ | CH₂OPh |
| H | CH(CH₃)CH₂SCH₃ | CF₃ | CH₂O(Ph-2-F) |
| H | CH(CH₃)CH₂SCH₃ | CF₃ | CH₂O(Ph-3-F) |
| H | CH(CH₃)CH₂SCH₃ | CF₃ | CH₂O(Ph-4-F) |
| H | CH(CH₃)CH₂SCH₃ | CF₃ | CH₂O(Ph-2-Cl) |
| H | CH(CH₃)CH₂SCH₃ | CF₃ | CH₂O(Ph-3-Cl) |
| H | CH(CH₃)CH₂SCH₃ | CF₃ | CH₂O(Ph-4-Cl) |
| H | CH(CH₃)CH₂SCH₃ | CF₃ | CH₂O(Ph-2-Br) |
| H | CH(CH₃)CH₂SCH₃ | CF₃ | CH₂O(Ph-3-Br) |
| H | CH(CH₃)CH₂SCH₃ | CF₃ | CH₂O(Ph-4-Br) |
| H | CH(CH₃)CH₂SCH₃ | CF₃ | CH₂O(Ph-2-CF₃) |
| H | CH(CH₃)CH₂SCH₃ | CF₃ | CH₂O(Ph-3-CF₃) |
| H | CH(CH₃)CH₂SCH₃ | CF₃ | CH₂O(Ph-4-CF₃) |
| H | CH(CH₃)CH₂SCH₃ | CF₃ | CH₂O(Ph-2-OCF₃) |
| H | CH(CH₃)CH₂SCH₃ | CF₃ | CH₂O(Ph-3-OCF₃) |
| H | CH(CH₃)CH₂SCH₃ | CF₃ | CH₂O(Ph-4-OCF₃) |
| H | CH(CH₃)CH₂SCH₃ | CF₃ | CF₂OPh |
| H | CH(CH₃)CH₂SCH₃ | CF₃ | CF₂O(Ph-2-F) |
| H | CH(CH₃)CH₂SCH₃ | CF₃ | CF₂O(Ph-3-F) |
| H | CH(CH₃)CH₂SCH₃ | CF₃ | CF₂O(Ph-4-F) |
| H | CH(CH₃)CH₂SCH₃ | CF₃ | CF₂O(Ph-2-Cl) |
| H | CH(CH₃)CH₂SCH₃ | CF₃ | CF₂O(Ph-3-Cl) |
| H | CH(CH₃)CH₂SCH₃ | CF₃ | CF₂O(Ph-4-Cl) |
| H | CH(CH₃)CH₂SCH₃ | CF₃ | CF₂O(Ph-2-Br) |
| H | CH(CH₃)CH₂SCH₃ | CF₃ | CF₂O(Ph-3-Br) |
| H | CH(CH₃)CH₂SCH₃ | CF₃ | CF₂O(Ph-4-Br) |
| H | CH(CH₃)CH₂SCH₃ | CF₃ | CF₂O(Ph-2-CF₃) |
| H | CH(CH₃)CH₂SCH₃ | CF₃ | CF₂O(Ph-3-CF₃) |
| H | CH(CH₃)CH₂SCH₃ | CF₃ | CF₂O(Ph-4-CF₃) |
| H | CH(CH₃)CH₂SCH₃ | CF₃ | CF₂O(Ph-2-OCF₃) |
| H | CH(CH₃)CH₂SCH₃ | CF₃ | CF₂O(Ph-3-OCF₃) |
| H | CH(CH₃)CH₂SCH₃ | CF₃ | CF₂O(Ph-4-OCF₃) |
| H | CH(CH₃)CH₂SCH₃ | CF₃ | CH₂S(Ph-2-Cl) |
| H | CH(CH₃)CH₂SCH₃ | CF₃ | CH₂S(Ph-3-Cl) |
| H | CH(CH₃)CH₂SCH₃ | CF₃ | CH₂S(Ph-4-Cl) |
| H | CH(CH₃)CH₂SCH₃ | CF₃ | CH₂SO₂(Ph-2-Cl) |
| H | CH(CH₃)CH₂SCH₃ | CF₃ | CH₂SO₂(Ph-3-Cl) |
| H | CH(CH₃)CH₂SCH₃ | CF₃ | CH₂SO₂(Ph-4-Cl) |
| H | CH(CH₃)CH₂SCH₃ | CF₃ | CH₂N(CH₃)₂ |
| H | CH(CH₃)CH₂SCH₃ | CF₃ | CH₂NH(Ph-2-Cl) |
| H | CH(CH₃)CH₂SCH₃ | CF₃ | CH₂NH(Ph-3-Cl) |
| H | CH(CH₃)CH₂SCH₃ | CF₃ | CH₂NH(Ph-4-Cl) |
| H | CH(CH₃)CH₂SCH₃ | CF₃ | CF₂C(O)OEt |
| H | CH(CH₃)CH₂SCH₃ | CF₃ | CH₂(L-5a) |
| H | CH(CH₃)CH₂SCH₃ | CF₃ | CH₂(L-14a) |
| H | CH(CH₃)CH₂SCH₃ | CF₃ | CH₂(L-24a) |
| H | CH(CH₃)CH₂SCH₃ | CF₃ | CH₂(L-36a) |
| H | CH(CH₃)CH₂SCH₃ | CF₃ | CH₂CH₂Ph |
| H | CH(CH₃)CH₂SCH₃ | CF₃ | CH₂CH₂(Ph-2-Cl) |
| H | CH(CH₃)CH₂SCH₃ | CF₃ | CH₂CH₂(Ph-3-Cl) |
| H | CH(CH₃)CH₂SCH₃ | CF₃ | CH₂CH₂(Ph-4-Cl) |
| H | CH(CH₃)CH₂SCH₃ | CF₃ | T-22 |
| H | CH(CH₃)CH₂SCH₃ | CF₃ | T-23 |
| H | CH(CH₃)CH₂SCH₃ | CF₃ | T-24 |
| H | CH(CH₃)CH₂SCH₃ | CF₃ | C(O)OEt |
| H | CH(CH₃)CH₂SCH₃ | CF₃ | C(O)OBu-t |
| H | CH(CH₃)CH₂SCH₃ | CF₃ | C(O)OCH₂CF₃ |
| H | CH(CH₃)CH₂SCH₃ | CF₃ | CH═CH(Ph-2-F) |
| H | CH(CH₃)CH₂SCH₃ | CF₃ | CH═CH(Ph-3-F) |
| H | CH(CH₃)CH₂SCH₃ | CF₃ | CH═CH(Ph-4-F) |
| H | CH(CH₃)CH₂SCH₃ | CF₃ | CH═CH(Ph-2-Cl) |
| H | CH(CH₃)CH₂SCH₃ | CF₃ | CH═CH(Ph-3-Cl) |
| H | CH(CH₃)CH₂SCH₃ | CF₃ | CH═CH(Ph-4-Cl) |
| H | CH(CH₃)CH₂SCH₃ | CF₃ | CH═CH(Ph-2-Br) |
| H | CH(CH₃)CH₂SCH₃ | CF₃ | CH═CH(Ph-3-Br) |
| H | CH(CH₃)CH₂SCH₃ | CF₃ | CH═CH(Ph-4-Br) |
| H | CH(CH₃)CH₂SCH₃ | CF₃ | CH═CH(Ph-2-CF₃) |
| H | CH(CH₃)CH₂SCH₃ | CF₃ | CH═CH(Ph-3-CF₃) |
| H | CH(CH₃)CH₂SCH₃ | CF₃ | CH═CH(Ph-4-CF₃) |
| H | CH(CH₃)CH₂SCH₃ | CF₃ | CH═CH(Ph-2-OCF₃) |
| H | CH(CH₃)CH₂SCH₃ | CF₃ | CH═CH(Ph-3-OCF₃) |
| H | CH(CH₃)CH₂SCH₃ | CF₃ | CH═CH(Ph-4-OCF₃) |
| H | CH(CH₃)CH₂SCH₃ | CF₃ | CH═CH(Ph-2-SCH₃) |
| H | CH(CH₃)CH₂SCH₃ | CF₃ | CH═CH(Ph-3-SCH₃) |
| H | CH(CH₃)CH₂SCH₃ | CF₃ | CH═CH(Ph-4-SCH₃) |
| H | CH(CH₃)CH₂SCH₃ | CF₃ | CH═CH(Ph-2-SO₂CH₃) |
| H | CH(CH₃)CH₂SCH₃ | CF₃ | CH═CH(Ph-3-SO₂CH₃) |
| H | CH(CH₃)CH₂SCH₃ | CF₃ | CH═CH(Ph-4-SO₂CH₃) |
| H | CH(CH₃)CH₂SCH₃ | CF₃ | CH═CH(Ph-3,4-F₂) |
| H | CH(CH₃)CH₂SCH₃ | CF₃ | CH═CH(Ph-3-F-4-Cl) |
| H | CH(CH₃)CH₂SCH₃ | CF₃ | CH═CH(Ph-3,4-Cl₂) |
| H | CH(CH₃)CH₂SCH₃ | CF₃ | CH═CH(Ph+3,4-Br₂) |
| H | CH(CH₃)CH₂SCH₃ | CF₃ | CH═CH(Ph-3-F-4-CF₃) |
| H | CH(CH₃)CH₂SCH₃ | CF₃ | CH═CH(Ph-3-Cl-4-OCF₃) |
| H | CH(CH₃)CH₂SCH₃ | CF₃ | CH═CH[Ph(-3-OCF₂O-4-)] |
| H | CH(CH₃)CH₂SCH₃ | CF₃ | Ph |
| H | CH(CH₃)CH₂SCH₃ | CF₃ | Ph-2-F |
| H | CH(CH₃)CH₂SCH₃ | CF₃ | Ph-3-F |
| H | CH(CH₃)CH₂SCH₃ | CF₃ | Ph-4-F |
| H | CH(CH₃)CH₂SCH₃ | CF₃ | Ph-2-Cl |
| H | CH(CH₃)CH₂SCH₃ | CF₃ | Ph-3-Cl |
| H | CH(CH₃)CH₂SCH₃ | CF₃ | Ph-4-Cl |
| H | CH(CH₃)CH₂SCH₃ | CF₃ | Ph-2-Br |
| H | CH(CH₃)CH₂SCH₃ | CF₃ | Ph-3-Br |
| H | CH(CH₃)CH₂SCH₃ | CF₃ | Ph-4-Br |
| H | CH(CH₃)CH₂SCH₃ | CF₃ | Ph-4-I |
| H | CH(CH₃)CH₂SCH₃ | CF₃ | Ph-4-Bu-t |
| H | CH(CH₃)CH₂SCH₃ | CF₃ | Ph-2-CF₃ |
| H | CH(CH₃)CH₂SCH₃ | CF₃ | Ph-3-CF₃ |
| H | CH(CH₃)CH₂SCH₃ | CF₃ | Ph-4-CF₃ |
| H | CH(CH₃)CH₂SCH₃ | CF₃ | Ph-4-OCH₃ |
| H | CH(CH₃)CH₂SCH₃ | CF₃ | Ph-4-OCHF₂ |
| H | CH(CH₃)CH₂SCH₃ | CF₃ | Ph-2-OCF₃ |
| H | CH(CH₃)CH₂SCH₃ | CF₃ | Ph-3-OCF₃ |
| H | CH(CH₃)CH₂SCH₃ | CF₃ | Ph-4-OCF₃ |
| H | CH(CH₃)CH₂SCH₃(R) | CF₃ | Ph-4-OCF₃ |
| H | CH(CH₃)CH₂SCH₃(S) | CF₃ | Ph-4-OCF₃ |
| H | CH(CH₃)CH₂SCH₃ | CF₃ | Ph-4-OCF₂Br |
| H | CH(CH₃)CH₂SCH₃ | CF₃ | Ph-4-OCH₂CF₃ |
| H | CH(CH₃)CH₂SCH₃ | CF₃ | Ph-4-OCF₂CHF₂ |
| H | CH(CH₃)CH₂SCH₃ | CF₃ | Ph-4-OCF₂CHFCl |
| H | CH(CH₃)CH₂SCH₃ | CF₃ | Ph-4-OCF₂CHFBr |
| H | CH(CH₃)CH₂SCH₃ | CF₃ | Ph-4-OCF₂CF₂Br |
| H | CH(CH₃)CH₂SCH₃ | CF₃ | Ph-4-OCF₂CFCl₂ |
| H | CH(CH₃)CH₂SCH₃ | CF₃ | Ph-4-OCF₂CCl₃ |
| H | CH(CH₃)CH₂SCH₃ | CF₃ | Ph-4-OCH₂CF₂CHF₂ |
| H | CH(CH₃)CH₂SCH₃ | CF₃ | Ph-3-OCF₂CHFCF₃ |
| H | CH(CH₃)CH₂SCH₃ | CF₃ | Ph-4-OCF₂CHFCF₃ |
| H | CH(CH₃)CH₂SCH₃ | CF₃ | Ph-4-OCH(CF₃)₂ |
| H | CH(CH₃)CH₂SCH₃ | CF₃ | Ph-4-OCF₂CFBrCF₃ |
| H | CH(CH₃)CH₂SCH₃ | CF₃ | Ph-3-OCF₂CHFOCF₃ |
| H | CH(CH₃)CH₂SCH₃ | CF₃ | Ph-4-OCF₂CHFOCF₃ |
| H | CH(CH₃)CH₂SCH₃ | CF₃ | Ph-4-OCF₂CHFOCF₂CF₂CF₃ |
| H | CH(CH₃)CH₂SCH₃ | CF₃ | Ph-4-OSO₂CH₃ |
| H | CH(CH₃)CH₂SCH₃ | CF₃ | Ph-4-OSO₂CF₃ |
| H | CH(CH₃)CH₂SCH₃ | CF₃ | Ph-3-O(Ph-4-Cl) |
| H | CH(CH₃)CH₂SCH₃ | CF₃ | Ph-4-O(Ph-4-Cl) |
| H | CH(CH₃)CH₂SCH₃ | CF₃ | Ph-4-O(Ph-4-Br) |
| H | CH(CH₃)CH₂SCH₃ | CF₃ | Ph-4-O(Ph-4-CF₃) |
| H | CH(CH₃)CH₂SCH₃ | CF₃ | Ph-4-O(L-45c)Br |
| H | CH(CH₃)CH₂SCH₃ | CF₃ | Ph-4-O(L-45c)CF₃ |
| H | CH(CH₃)CH₂SCH₃ | CF₃ | Ph-3-O(L-45e) |
| H | CH(CH₃)CH₂SCH₃ | CF₃ | Ph-4-O(L-45e) |
| H | CH(CH₃)CH₂SCH₃ | CF₃ | Ph-4-O(L-48b)Br |
| H | CH(CH₃)CH₂SCH₃ | CF₃ | Ph-4-SCH₃ |
| H | CH(CH₃)CH₂SCH₃ | CF₃ | Ph-4-SO₂CH₃ |
| H | CH(CH₃)CH₂SCH₃ | CF₃ | Ph-3-S(Ph-4-Cl) |
| H | CH(CH₃)CH₂SCH₃ | CF₃ | Ph-4-S(Ph-4-Cl) |
| H | CH(CH₃)CH₂SCH₃ | CF₃ | Ph-4-S(Ph-4-Br) |
| H | CH(CH₃)CH₂SCH₃ | CF₃ | Ph-4-S(Ph-4-CF₃) |
| H | CH(CH₃)CH₂SCH₃ | CF₃ | Ph-4-S(L-45c)Br |
| H | CH(CH₃)CH₂SCH₃ | CF₃ | Ph-4-S(L-45c)CF₃ |
| H | CH(CH₃)CH₂SCH₃ | CF₃ | Ph-4-S(L-45e) |
| H | CH(CH₃)CH₂SCH₃ | CF₃ | Ph-4-S(L-48b)Br |
| H | CH(CH₃)CH₂SCH₃ | CF₃ | Ph-4-NO₂ |
| H | CH(CH₃)CH₂SCH₃ | CF₃ | Ph-4-CN |
| H | CH(CH₃)CH₂SCH₃ | CF₃ | Ph-2,3-F₂ |
| H | CH(CH₃)CH₂SCH₃ | CF₃ | Ph-2,4-F₂ |
| H | CH(CH₃)CH₂SCH₃ | CF₃ | Ph-3,4-F₂ |
| H | CH(CH₃)CH₂SCH₃ | CF₃ | Ph-2,5-F₂ |
| H | CH(CH₃)CH₂SCH₃ | CF₃ | Ph-3,5-F₂ |
| H | CH(CH₃)CH₂SCH₃ | CF₃ | Ph-2-Cl-4-F |
| H | CH(CH₃)CH₂SCH₃ | CF₃ | Ph-2-F-3-Cl |
| H | CH(CH₃)CH₂SCH₃ | CF₃ | Ph-3-Cl-4-F |
| H | CH(CH₃)CH₂SCH₃ | CF₃ | Ph-2-F-4-Cl |
| H | CH(CH₃)CH₂SCH₃ | CF₃ | Ph-3-F-4-Cl |
| H | CH(CH₃)CH₂SCH₃ | CF₃ | Ph-2,3-Cl₂ |
| H | CH(CH₃)CH₂SCH₃ | CF₃ | Ph-2,4-Cl₂ |
| H | CH(CH₃)CH₂SCH₃ | CF₃ | Ph-2,5-Cl₂ |
| H | CH(CH₃)CH₂SCH₃ | CF₃ | Ph-3,4-Cl₂ |
| H | CH(CH₃)CH₂SCH₃ | CF₃ | Ph-3,5-Cl₂ |
| H | CH(CH₃)CH₂SCH₃ | CF₃ | Ph-3-Br-4-F |
| H | CH(CH₃)CH₂SCH₃ | CF₃ | Ph-2-F-4-Br |
| H | CH(CH₃)CH₂SCH₃ | CF₃ | Ph-2-F-5-Br |
| H | CH(CH₃)CH₂SCH₃ | CF₃ | Ph-3,4-Br₂ |
| H | CH(CH₃)CH₂SCH₃ | CF₃ | Ph-3,5-Br₂ |
| H | CH(CH₃)CH₂SCH₃ | CF₃ | Ph-3-CH₃-4-F |
| H | CH(CH₃)CH₂SCH₃ | CF₃ | Ph-3-F-4-CH₃ |
| H | CH(CH₃)CH₂SCH₃ | CF₃ | Ph-2-F-5-CH₃ |
| H | CH(CH₃)CH₂SCH₃ | CF₃ | Ph-2,4-(CH₃)₂ |
| H | CH(CH₃)CH₂SCH₃ | CF₃ | Ph-3,4-(CH₃)₂ |
| H | CH(CH₃)CH₂SCH₃ | CF₃ | Ph-2-F-3-CF₃ |
| H | CH(CH₃)CH₂SCH₃ | CF₃ | Ph-3-CF₃-4-F |
| H | CH(CH₃)CH₂SCH₃ | CF₃ | Ph-3-CF₃-4-Cl |
| H | CH(CH₃)CH₂SCH₃ | CF₃ | Ph-2-F-4-CF₃ |
| H | CH(CH₃)CH₂SCH₃ | CF₃ | Ph-3-F-4-CF₃ |
| H | CH(CH₃)CH₂SCH₃ | CF₃ | Ph-2-F-5-CF₃ |
| H | CH(CH₃)CH₂SCH₃ | CF₃ | Ph-3-F-5-CF₃ |
| H | CH(CH₃)CH₂SCH₃ | CF₃ | Ph-2-Cl-4-CF₃ |
| H | CH(CH₃)CH₂SCH₃ | CF₃ | Ph-3,5-(CF₃)₂ |
| H | CH(CH₃)CH₂SCH₃ | CF₃ | Ph-3-Br-4-OCH₃ |
| H | CH(CH₃)CH₂SCH₃ | CF₃ | Ph-3-F-4-OCHF₂ |
| H | CH(CH₃)CH₂SCH₃ | CF₃ | Ph-3-Cl-4-OCHF₂ |
| H | CH(CH₃)CH₂SCH₃ | CF₃ | Ph-3-Br-4-OCHF₂ |
| H | CH(CH₃)CH₂SCH₃ | CF₃ | Ph-3-F-4-OCF₃ |
| H | CH(CH₃)CH₂SCH₃ | CF₃ | Ph-3-Cl-4-OCF₃ |
| H | CH(CH₃)CH₂SCH₃ | CF₃ | Ph-3-Br-4-OCF₃ |
| H | CH(CH₃)CH₂SCH₃ | CF₃ | Ph-3-F-4-OCF₂Br |
| H | CH(CH₃)CH₂SCH₃ | CF₃ | Ph-3-Cl-4-OCF₂Br |
| H | CH(CH₃)CH₂SCH₃ | CF₃ | Ph-3-Br-4-OCF₂Br |
| H | CH(CH₃)CH₂SCH₃ | CF₃ | Ph-3-F-4-OCF₂CHF₂ |
| H | CH(CH₃)CH₂SCH₃ | CF₃ | Ph-3-Cl-4-OCF₂CHF₂ |
| H | CH(CH₃)CH₂SCH₃ | CF₃ | Ph-3-Br-4-OCF₂CHF₂ |
| H | CH(CH₃)CH₂SCH₃ | CF₃ | Ph-3-F-4-OCF₂CHFCl |
| H | CH(CH₃)CH₂SCH₃ | CF₃ | Ph-3-Cl-4-OCF₂CHFCl |
| H | CH(CH₃)CH₂SCH₃ | CF₃ | Ph-3-Br-4-OCF₂CHFCl |
| H | CH(CH₃)CH₂SCH₃ | CF₃ | Ph-3-F-4-OCF₂CHFCF₃ |
| H | CH(CH₃)CH₂SCH₃ | CF₃ | Ph-3-Cl-4-OCF₂CHFCF₃ |
| H | CH(CH₃)CH₂SCH₃ | CF₃ | Ph-3-Br-4-OCF₂CHFCF₃ |
| H | CH(CH₃)CH₂SCH₃ | CF₃ | Ph-3-F-4-OCF₂CHFOCF₃ |
| H | CH(CH₃)CH₂SCH₃ | CF₃ | Ph-3-Cl-4-OCF₂CHFOCF₃ |
| H | CH(CH₃)CH₂SCH₃ | CF₃ | Ph-3-Br-4-OCF₂CHFOCF₃ |
| H | CH(CH₃)CH₂SCH₃ | CF₃ | Ph-3-CH₃-4-OCF₂CHFOCF₃ |
| H | CH(CH₃)CH₂SCH₃ | CF₃ | Ph-3-F-4-OCF₂CHFOCF₂CF₂CF₃ |
| H | CH(CH₃)CH₂SCH₃ | CF₃ | Ph-3-Cl-4-OCF₂CHFOCF₂CF₂CF₃ |
| H | CH(CH₃)CH₂SCH₃ | CF₃ | Ph-3-Br-4-OCF₂CHFOCF₂CF₂CF₃ |
| H | CH(CH₃)CH₂SCH₃ | CF₃ | Ph(-3-OCF₂O-4-) |
| H | CH(CH₃)CH₂SCH₃ | CF₃ | Ph(-3-OCF₂CF₂O-4-) |
| H | CH(CH₃)CH₂SCH₃ | CF₃ | Ph-3-OPh-4-F |
| H | CH(CH₃)CH₂SCH₃ | CF₃ | Ph-3-NO₂-4-F |
| H | CH(CH₃)CH₂SCH₃ | CF₃ | Ph-3-NO₂-4-Cl |
| H | CH(CH₃)CH₂SCH₃ | CF₃ | Ph-2-F-5-NO₂ |
| H | CH(CH₃)CH₂SCH₃ | CF₃ | Ph-3-CN-4-F |
| H | CH(CH₃)CH₂SCH₃ | CF₃ | Ph-2,3,4-F₃ |
| H | CH(CH₃)CH₂SCH₃ | CF₃ | Ph-2,3,5-F₃ |
| H | CH(CH₃)CH₂SCH₃ | CF₃ | Ph-2,4,5-F₃ |
| H | CH(CH₃)CH₂SCH₃ | CF₃ | Ph-3,4,5-F₃ |
| H | CH(CH₃)CH₂SCH₃ | CF₃ | Ph-2,3-F₂-4-CH₃ |
| H | CH(CH₃)CH₂SCH₃ | CF₃ | Ph-2,3-F₂-4-CF₃ |
| H | CH(CH₃)CH₂SCH₃ | CF₃ | Ph-3,4-F₂-5-CF₃ |
| H | CH(CH₃)CH₂SCH₃ | CF₃ | Ph-2-F-3-Cl-5-CF₃ |
| H | CH(CH₃)CH₂SCH₃ | CF₃ | Ph-3,5-Cl₂-4-OCH₃ |
| H | CH(CH₃)CH₂SCH₃ | CF₃ | 1-Naph |
| H | CH(CH₃)CH₂SCH₃ | CF₃ | 2-Naph |
| H | CH(CH₃)CH₂SCH₃ | CF₃ | (L-1b)Br |
| H | CH(CH₃)CH₂SCH₃ | CF₃ | (L-1c)Cl |
| H | CH(CH₃)CH₂SCH₃ | CF₃ | (L-1c)Br |
| H | CH(CH₃)CH₂SCH₃ | CF₃ | (L-1c)I |
| H | CH(CH₃)CH₂SCH₃ | CF₃ | (L-1c)CF₃ |
| H | CH(CH₃)CH₂SCH₃ | CF₃ | (L-2b)Br |
| H | CH(CH₃)CH₂SCH₃ | CF₃ | (L-3b)Cl |
| H | CH(CH₃)CH₂SCH₃ | CF₃ | (L-3b)Br |
| H | CH(CH₃)CH₂SCH₃ | CF₃ | (L-3c)F |
| H | CH(CH₃)CH₂SCH₃ | CF₃ | (L-3c)Cl |
| H | CH(CH₃)CH₂SCH₃ | CF₃ | (L-3c)Br |
| H | CH(CH₃)CH₂SCH₃ | CF₃ | (L-3c)I |
| H | CH(CH₃)CH₂SCH₃ | CF₃ | (L-3c)CF₃ |
| H | CH(CH₃)CH₂SCH₃ | CF₃ | (L-3c)CN |
| H | CH(CH₃)CH₂SCH₃ | CF₃ | L-3d |
| H | CH(CH₃)CH₂SCH₃ | CF₃ | (L-4b)Cl |
| H | CH(CH₃)CH₂SCH₃ | CF₃ | (L-4b)Br |
| H | CH(CH₃)CH₂SCH₃ | CF₃ | (L-4b)CN |
| H | CH(CH₃)CH₂SCH₃ | CF₃ | (L-10b)Cl |
| H | CH(CH₃)CH₂SCH₃ | CF₃ | (L-10b)Br |
| H | CH(CH₃)CH₂SCH₃ | CF₃ | (L-15b)CF₃ |
| H | CH(CH₃)CH₂SCH₃ | CF₃ | (L-16a)CHF₂ |
| H | CH(CH₃)CH₂SCH₃ | CF₃ | (L-16a)CF₂Br |
| H | CH(CH₃)CH₂SCH₃ | CF₃ | (L-17a)Cl |
| H | CH(CH₃)CH₂SCH₃ | CF₃ | (L-21b)Cl |
| H | CH(CH₃)CH₂SCH₃ | CF₃ | (L-21b)Br |
| H | CH(CH₃)CH₂SCH₃ | CF₃ | (L-21b)I |
| H | CH(CH₃)CH₂SCH₃ | CF₃ | (L-21b)CF₃ |
| H | CH(CH₃)CH₂SCH₃ | CF₃ | (L-22b)Cl |
| H | CH(CH₃)CH₂SCH₃ | CF₃ | (L-22b)Br |
| H | CH(CH₃)CH₂SCH₃ | CF₃ | (L-23b)Cl |
| H | CH(CH₃)CH₂SCH₃ | CF₃ | (L-23b)Br |
| H | CH(CH₃)CH₂SCH₃ | CF₃ | (L-23c)Cl |
| H | CH(CH₃)CH₂SCH₃ | CF₃ | (L-23c)Br |
| H | CH(CH₃)CH₂SCH₃ | CF₃ | (L-31a)Cl |
| H | CH(CH₃)CH₂SCH₃ | CF₃ | (L-31a)Br |
| H | CH(CH₃)CH₂SCH₃ | CF₃ | (L-45c)F |
| H | CH(CH₃)CH₂SCH₃ | CF₃ | (L-45c)Cl |
| H | CH(CH₃)CH₂SCH₃ | CF₃ | (L-45c)Br |
| H | CH(CH₃)CH₂SCH₃ | CF₃ | (L-45c)I |
| H | CH(CH₃)CH₂SCH₃ | CF₃ | (L-45c)CF₃ |
| H | CH(CH₃)CH₂SCH₃ | CF₃ | L-45e |
| H | CH(CH₃)CH₂SCH₃ | CF₃ | L-45f |
| H | CH(CH₃)CH₂SCH₃ | CF₃ | (L-46c)F |
| H | CH(CH₃)CH₂SCH₃ | CF₃ | (L-46c)Cl |
| H | CH(CH₃)CH₂SCH₃ | CF₃ | (L-46c)Br |
| H | CH(CH₃)CH₂SCH₃ | CF₃ | (L-46c)I |
| H | CH(CH₃)CH₂SCH₃ | CF₃ | (L-46c)CF₃ |
| H | CH(CH₃)CH₂SCH₃ | CF₃ | (L-46c)OCH₂CF₃ |
| H | CH(CH₃)CH₂SCH₃ | CF₃ | (L-46c)OCH(CF₃)₂ |
| H | CH(CH₃)CH₂SCH₃ | CF₃ | L-46d |
| H | CH(CH₃)CH₂SCH₃ | CF₃ | L-47a |
| H | CH(CH₃)CH₂SCH₃ | CF₃ | L-47d |
| H | CH(CH₃)CH₂SCH₃ | CF₃ | (L-48b)Br |
| H | CH(CH₃)CH₂SCH₃ | CF₃ | (L-50b)Cl |
| H | CH(CH₃)CH₂SCH₃ | CF₃ | (L-50b)Br |
| H | CH(CH₃)CH₂SCH₃ | CF₃ | (L-51b)Cl |
| H | CH(CH₃)CH₂SCH₃ | CF₂Cl | Ph-4-Cl |
| H | CH(CH₃)CH₂SCH₃ | CF₂Cl | Ph-4-Br |
| H | CH(CH₃)CH₂SCH₃ | CF₂Cl | Ph-4-CF₃ |
| H | CH(CH₃)CH₂SCH₃ | CF₂Cl | Ph-4-OCF₃ |
| H | CH(CH₃)CH₂SCH₃ | CF₂Cl | Ph-4-OCF₂Br |
| H | CH(CH₃)CH₂SCH₃ | CF₂Cl | Ph-4-OCF₂CHFCF₃ |
| H | CH(CH₃)CH₂SCH₃ | CF₂Cl | Ph-4-OCF₂CHFOCF₃ |
| H | CH(CH₃)CH₂SCH₃ | CF₂Cl | Ph-4-OCF₂CHFOCF₂CF₂CF₃ |
| H | CH(CH₃)CH₂SCH₃ | CF₂Cl | Ph-4-O(L-45g) |
| H | CH(CH₃)CH₂SCH₃ | CF₂Cl | Ph-3,4-Cl₂ |
| H | CH(CH₃)CH₂SCH₃ | CF₂Cl | Ph(-3-OCF₂O-4-) |
| H | CH(CH₃)CH₂SCH₃ | CF₂Cl | Ph(-3-OCF₂CF₂O-4-) |
| H | CH(CH₃)CH₂SCH₃ | CF₂Cl | (L-45c)CF₃ |
| H | CH(CH₃)CH₂SCH₃ | CF₂Cl | L-45f |
| H | CH(CH₃)CH₂SCH₃ | CF₂Cl | (L-46c)CF₃ |
| H | CH(CH₃)CH₂SCH₃ | CF₂Cl | L-46d |
| H | CH(CH₃)CH₂SCH₃ | CFCl₂ | (L-45c)CF₃ |
| H | CH(CH₃)CH₂SCH₃ | CF₂Br | Ph-4-Cl |
| H | CH(CH₃)CH₂SCH₃ | CF₂Br | Ph-4-Br |
| H | CH(CH₃)CH₂SCH₃ | CF₂Br | Ph-4-CF₃ |
| H | CH(CH₃)CH₂SCH₃ | CF₂Br | Ph-4-OCF₃ |
| H | CH(CH₃)CH₂SCH₃ | CF₂Br | Ph-4-OCF₂Br |
| H | CH(CH₃)CH₂SCH₃ | CF₂Br | Ph-4-OCF₂CHFCF₃ |
| H | CH(CH₃)CH₂SCH₃ | CF₂Br | Ph-4-OCF₂CHFOCF₃ |
| H | CH(CH₃)CH₂SCH₃ | CF₂Br | Ph-4-OCF₂CHFOCF₂CF₂CF₃ |
| H | CH(CH₃)CH₂SCH₃ | CF₂Br | Ph-4-O(L-45g) |
| H | CH(CH₃)CH₂SCH₃ | CF₂Br | Ph-3,4-Cl₂ |
| H | CH(CH₃)CH₂SCH₃ | CF₂Br | Ph(-3-OCF₂O-4-) |
| H | CH(CH₃)CH₂SCH₃ | CF₂Br | Ph(-3-OCF₂CF₂O-4-) |
| H | CH(CH₃)CH₂SCH₃ | CF₂Br | (L-45c)CF₃ |
| H | CH(CH₃)CH₂SCH₃ | CF₂Br | L-45f |
| H | CH(CH₃)CH₂SCH₃ | CF₂Br | (L-46c)CF₃ |
| H | CH(CH₃)CH₂SCH₃ | CF₂Br | L-46d |
| H | CH(CH₃)CH₂SCH₃ | CFClBr | Ph-4-Br |
| H | CH(CH₃)CH₂SCH₃ | CFBr₂ | Ph-4-CF₃ |
| H | CH(CH₃)CH₂SCH₃ | CF₂CHF₂ | Ph-4-OCF₃ |
| H | CH(CH₃)CH₂SCH₃ | CF₂CF₃ | Ph-4-Cl |
| H | CH(CH₃)CH₂SCH₃ | CF₂CF₃ | Ph-4-Br |
| H | CH(CH₃)CH₂SCH₃ | CF₂CF₃ | Ph-4-CF₃ |
| H | CH(CH₃)CH₂SCH₃ | CF₂CF₃ | Ph-4-OCF₃ |
| H | CH(CH₃)CH₂SCH₃ | CF₂CF₃ | Ph-4-OCF₂Br |
| H | CH(CH₃)CH₂SCH₃ | CF₂CF₃ | Ph-4-OCF₂CHFCF₃ |
| H | CH(CH₃)CH₂SCH₃ | CF₂CF₃ | Ph-4-OCF₂CHFOCF₃ |
| H | CH(CH₃)CH₂SCH₃ | CF₂CF₃ | Ph-4-OCF₂CHFOCF₂CF₂CF₃ |
| H | CH(CH₃)CH₂SCH₃ | CF₂CF₃ | Ph-4-O(L-45g) |
| H | CH(CH₃)CH₂SCH₃ | CF₂CF₃ | Ph-3,4-Cl₂ |
| H | CH(CH₃)CH₂SCH₃ | CF₂CF₃ | Ph(-3-OCF₂O-4-) |
| H | CH(CH₃)CH₂SCH₃ | CF₂CF₃ | Ph(-3-OCF₂CF₂O-4-) |
| H | CH(CH₃)CH₂SCH₃ | CF₂CF₃ | (L-45c)CF₃ |
| H | CH(CH₃)CH₂SCH₃ | CF₂CF₃ | L-45f |
| H | CH(CH₃)CH₂SCH₃ | CF₂CF₃ | (L-46c)CF₃ |
| H | CH(CH₃)CH₂SCH₃ | CF₂CF₃ | L-46d |
| H | CH(CH₃)CH₂SCH₃ | CF₂CF₂Cl | (L-45c)CF₃ |
| H | CH(CH₃)CH₂SCH₃ | CFClCF₃ | Ph-4-Br |
| H | CH(CH₃)CH₂SCH₃ | CFClCF₂Cl | Ph-4-CF₃ |
| H | CH(CH₃)CH₂SCH₃ | CF₂CF₂Br | Ph-4-OCF₃ |
| H | CH(CH₃)CH₂SCH₃ | CFBrCF₃ | (L-45c)CF₃ |
| H | CH(CH₃)CH₂SCH₃ | CF₂CHFCF₃ | Ph-4-Br |
| H | CH(CH₃)CH₂SCH₃ | CF₂CF₂CF₃ | Ph-4-Cl |
| H | CH(CH₃)CH₂SCH₃ | CF₂CF₂CF₃ | Ph-4-Br |
| H | CH(CH₃)CH₂SCH₃ | CF₂CF₂CF₃ | Ph-4-CF₃ |
| H | CH(CH₃)CH₂SCH₃ | CF₂CF₂CF₃ | Ph-4-OCF₃ |
| H | CH(CH₃)CH₂SCH₃ | CF₂CF₂CF₃ | Ph-4-OCF₂Br |
| H | CH(CH₃)CH₂SCH₃ | CF₂CF₂CF₃ | Ph-4-OCF₂CHFCF₃ |
| H | CH(CH₃)CH₂SCH₃ | CF₂CF₂CF₃ | Ph-4-OCF₂CHFOCF₃ |
| H | CH(CH₃)CH₂SCH₃ | CF₂CF₂CF₃ | Ph-4-OCF₂CHFOCF₂CF₂CF₃ |
| H | CH(CH₃)CH₂SCH₃ | CF₂CF₂CF₃ | Ph-4-O(L-45g) |
| H | CH(CH₃)CH₂SCH₃ | CF₂CF₂CF₃ | Ph-3,4-Cl₂ |
| H | CH(CH₃)CH₂SCH₃ | CF₂CF₂CF₃ | Ph(-3-OCF₂O-4-) |
| H | CH(CH₃)CH₂SCH₃ | CF₂CF₂CF₃ | Ph(-3-OCF₂CF₂O-4-) |
| H | CH(CH₃)CH₂SCH₃ | CF₂CF₂CF₃ | (L-45c)CF₃ |
| H | CH(CH₃)CH₂SCH₃ | CF₂CF₂CF₃ | L-45f |
| H | CH(CH₃)CH₂SCH₃ | CF₂CF₂CF₃ | (L-46c)CF₃ |
| H | CH(CH₃)CH₂SCH₃ | CF₂CF₂CF₃ | L-46d |
| H | CH(CH₃)CH₂SCH₃ | CF(CF₃)₂ | Ph-4-CF₃ |
| H | CH(CH₃)CH₂SCH₃ | CF₂CF₂CF₂CF₃ | Ph-4-OCF₃ |
| H | CH(CH₃)CH₂SCH₃ | T-1 | (L-45c)CF₃ |
| H | CH(CH₃)CH₂SCH₃ | T-2 | Ph-4-Br |
| H | CH(CH₃)CH₂SCH₃ | CH₂OCH₃ | Ph-4-CF₃ |
| H | CH(CH₃)CH₂SCH₃ | CH₂OEt | Ph-4-OCF₃ |
| H | CH(CH₃)CH₂SCH₃ | CH₂OCH₂CF₃ | (L-45c)CF₃ |
| H | CH(CH₃)CH₂SCH₃ | CH₂OCH(CF₃)₂ | Ph-4-Br |
| H | CH(CH₃)CH₂SCH₃ | CF₂OCH₃ | Ph-4-Cl |
| H | CH(CH₃)CH₂SCH₃ | CF₂OCH₃ | Ph-4-Br |
| H | CH(CH₃)CH₂SCH₃ | CF₂OCH₃ | Ph-4-CF₃ |
| H | CH(CH₃)CH₂SCH₃ | CF₂OCH₃ | Ph-4-OCF₃ |
| H | CH(CH₃)CH₂SCH₃ | CF₂OCH₃ | Ph-4-OCF₂Br |
| H | CH(CH₃)CH₂SCH₃ | CF₂OCH₃ | Ph-4-OCF₂CHFCF₃ |
| H | CH(CH₃)CH₂SCH₃ | CF₂OCH₃ | Ph-4-OCF₂CHFOCF₃ |
| H | CH(CH₃)CH₂SCH₃ | CF₂OCH₃ | Ph-4-OCF₂CHFOCF₂CF₂CF₃ |
| H | CH(CH₃)CH₂SCH₃ | CF₂OCH₃ | Ph-4-O(L-45g) |
| H | CH(CH₃)CH₂SCH₃ | CF₂OCH₃ | Ph-3,4-Cl₂ |
| H | CH(CH₃)CH₂SCH₃ | CF₂OCH₃ | Ph(-3-OCF₂O-4-) |
| H | CH(CH₃)CH₂SCH₃ | CF₂OCH₃ | Ph(-3-OCF₂CF₂O-4-) |
| H | CH(CH₃)CH₂SCH₃ | CF₂OCH₃ | (L-45c)CF₃ |
| H | CH(CH₃)CH₂SCH₃ | CF₂OCH₃ | L-45f |
| H | CH(CH₃)CH₂SCH₃ | CF₂OCH₃ | (L-46c)CF₃ |
| H | CH(CH₃)CH₂SCH₃ | CF₂OCH₃ | L-46d |
| H | CH(CH₃)CH₂SCH₃ | CH₂SCH₃ | Ph-4-CF₃ |
| H | CH(CH₃)CH₂SCH₃ | CH₂SO₂CH₃ | Ph-4-OCF₃ |
| H | CH(CH₃)CH₂SCH₃ | CH₂SCF₃ | (L-45c)CF₃ |
| H | CH(CH₃)CH₂SCH₃ | CF₂SCH₃ | Ph-4-Cl |
| H | CH(CH₃)CH₂SCH₃ | CF₂SCH₃ | Ph-4-Br |
| H | CH(CH₃)CH₂SCH₃ | CF₂SCH₃ | Ph-4-CF₃ |
| H | CH(CH₃)CH₂SCH₃ | CF₂SCH₃ | Ph-4-OCF₃ |
| H | CH(CH₃)CH₂SCH₃ | CF₂SCH₃ | Ph-4-OCF₂Br |
| H | CH(CH₃)CH₂SCH₃ | CF₂SCH₃ | Ph-4-OCF₂CHFCF₃ |
| H | CH(CH₃)CH₂SCH₃ | CF₂SCH₃ | Ph-4-OCF₂CHFOCF₃ |
| H | CH(CH₃)CH₂SCH₃ | CF₂SCH₃ | Ph-4-OCF₂CHFOCF₂CF₂CF₃ |
| H | CH(CH₃)CH₂SCH₃ | CF₂SCH₃ | Ph-4-O(L-45g) |
| H | CH(CH₃)CH₂SCH₃ | CF₂SCH₃ | Ph-3,4-Cl₂ |
| H | CH(CH₃)CH₂SCH₃ | CF₂SCH₃ | Ph(-3-OCF₂O-4-) |
| H | CH(CH₃)CH₂SCH₃ | CF₂SCH₃ | Ph(-3-OCF₂CF₂O-4-) |
| H | CH(CH₃)CH₂SCH₃ | CF₂SCH₃ | (L-45c)CF₃ |
| H | CH(CH₃)CH₂SCH₃ | CF₂SCH₃ | L-45f |
| H | CH(CH₃)CH₂SCH₃ | CF₂SCH₃ | (L-46c)CF₃ |
| H | CH(CH₃)CH₂SCH₃ | CF₂SCH₃ | L-46d |
| H | CH(CH₃)CH₂SCH₃ | CF₂SEt | Ph-4-Br |
| H | CH(CH₃)CH₂SCH₃ | CF₂SPr-n | Ph-4-CF₃ |
| H | CH(CH₃)CH₂SCH₃ | CF₂SPr-i | Ph-4-OCF₃ |
| H | CH(CH₃)CH₂SCH₃ | CF₂SCH₂Ph | (L-45c)CF₃ |
| H | CH(CH₃)CH₂SCH₃ | CF₂SPh | Ph-4-Br |
| H | CH(CH₃)CH₂SCH₃ | CF₂C(O)OEt | Ph-4-CF₃ |
| H | CH(CH₃)CH₂SCH₃ | CF₂SO₂N(CH₃)₂ | Ph-4-OCF₃ |
| H | CH(CH₃)CH₂SCH₃ | C(O)OEt | (L-45c)CF₃ |
| H | CH(CH₃)CH₂SCH₃ | C(O)SEt | Ph-4-Br |
| CH₃ | CH(CH₃)CH₂SCH₃ | CF₃ | Ph-4-CF₃ |
| Et | CH(CH₃)CH₂SCH₃ | CF₃ | Ph-4-OCF₃ |
| H | CH(CH₃)CH₂S(O)CH₃ | CH₃ | Ph-4-Br |
| H | CH(CH₃)CH₂S(O)CH₃ | CH₃ | Ph-4-CF₃ |
| H | CH(CH₃)CH₂S(O)CH₃ | CH₃ | Ph-4-OCF₃ |
| H | CH(CH₃)CH₂S(O)CH₃ | CH₃ | (L-45c)CF₃ |
| H | CH(CH₃)CH₂S(O)CH₃ | CF₃ | CH₂O(Ph-4-Cl) |
| H | CH(CH₃)CH₂S(O)CH₃ | CF₃ | CH₂O(Ph-4-Br) |
| H | CH(CH₃)CH₂S(O)CH₃ | CF₃ | CH₂O(Ph-4-CF₃) |
| H | CH(CH₃)CH₂S(O)CH₃ | CF₃ | CH₂O(Ph-4-OCF₃) |
| H | CH(CH₃)CH₂S(O)CH₃ | CF₃ | CF₂O(Ph-4-Cl) |
| H | CH(CH₃)CH₂S(O)CH₃ | CF₃ | CF₂O(Ph-4-Br) |
| H | CH(CH₃)CH₂S(O)CH₃ | CF₃ | CF₂O(Ph-4-CF₃) |
| H | CH(CH₃)CH₂S(O)CH₃ | CF₃ | CF₂O(Ph-4-OCF₃) |
| H | CH(CH₃)CH₂S(O)CH₃ | CF₃ | CH═CH(Ph-4-F) |
| H | CH(CH₃)CH₂S(O)CH₃ | CF₃ | CH═CH(Ph-4-Cl) |
| H | CH(CH₃)CH₂S(O)CH₃ | CF₃ | CH═CH(Ph-4-Br) |
| H | CH(CH₃)CH₂S(O)CH₃ | CF₃ | CH═CH(Ph-4-CF₃) |
| H | CH(CH₃)CH₂S(O)CH₃ | CF₃ | CH═CH(Ph-4-OCF₃) |
| H | CH(CH₃)CH₂S(O)CH₃ | CF₃ | CH═CH(Ph-4-SCH₃) |
| H | CH(CH₃)CH₂S(O)CH₃ | CF₃ | CH═CH(Ph-4-SO₂CH₃) |
| H | CH(CH₃)CH₂S(O)CH₃ | CF₃ | CH═CH(Ph-3,4-Cl₂) |
| H | CH(CH₃)CH₂S(O)CH₃ | CF₃ | Ph-4-F |
| H | CH(CH₃)CH₂S(O)CH₃ | CF₃ | Ph-2-Cl |
| H | CH(CH₃)CH₂S(O)CH₃ | CF₃ | Ph-3-Cl |
| H | CH(CH₃)CH₂S(O)CH₃ | CF₃ | Ph-4-Cl |
| H | CH(CH₃)CH₂S(O)CH₃ | CF₃ | Ph-4-Br |
| H | CH(CH₃)CH₂S(O)CH₃ | CF₃ | Ph-4-I |
| H | CH(CH₃)CH₂S(O)CH₃ | CF₃ | Ph-4-CF₃ |
| H | CH(CH₃)CH₂S(O)CH₃ | CF₃ | Ph-4-OCHF₂ |
| H | CH(CH₃)CH₂S(O)CH₃ | CF₃ | Ph-4-OCF₃ |
| H | CH(CH₃)CH₂S(O)CH₃ | CF₃ | Ph-4-OCF₂Br |
| H | CH(CH₃)CH₂S(O)CH₃ | CF₃ | Ph-4-OCH₂CF₃ |
| H | CH(CH₃)CH₂S(O)CH₃ | CF₃ | Ph-4-OCF₂CHF₂ |
| H | CH(CH₃)CH₂S(O)CH₃ | CF₃ | Ph-4-OCF₂CHFCl |
| H | CH(CH₃)CH₂S(O)CH₃ | CF₃ | Ph-4-OCF₂CHFCF₃ |
| H | CH(CH₃)CH₂S(O)CH₃ | CF₃ | Ph-4-OCF₂CHFOCF₃ |
| H | CH(CH₃)CH₂S(O)CH₃ | CF₃ | Ph-4-OCF₂CHFOCF₂CF₂CF₃ |
| H | CH(CH₃)CH₂S(O)CH₃ | CF₃ | Ph-4-OSO₂CH₃ |
| H | CH(CH₃)CH₂S(O)CH₃ | CF₃ | Ph-4-OSO₂CF₃ |
| H | CH(CH₃)CH₂S(O)CH₃ | CF₃ | Ph-4-O(L-45e) |
| H | CH(CH₃)CH₂S(O)CH₃ | CF₃ | Ph-2,4-F₂ |
| H | CH(CH₃)CH₂S(O)CH₃ | CF₃ | Ph-3,4-F₂ |
| H | CH(CH₃)CH₂S(O)CH₃ | CF₃ | Ph-3-Cl-4-F |
| H | CH(CH₃)CH₂S(O)CH₃ | CF₃ | Ph-2-F-4-Cl |
| H | CH(CH₃)CH₂S(O)CH₃ | CF₃ | Ph-3-F-4-Cl |
| H | CH(CH₃)CH₂S(O)CH₃ | CF₃ | Ph-2,4-Cl₂ |
| H | CH(CH₃)CH₂S(O)CH₃ | CF₃ | Ph-3,4-Cl₂ |
| H | CH(CH₃)CH₂S(O)CH₃ | CF₃ | Ph-3,5-Cl₂ |
| H | CH(CH₃)CH₂S(O)CH₃ | CF₃ | Ph-3-Br-4-F |
| H | CH(CH₃)CH₂S(O)CH₃ | CF₃ | Ph-2-F-4-Br |
| H | CH(CH₃)CH₂S(O)CH₃ | CF₃ | Ph-3,4-Br₂ |
| H | CH(CH₃)CH₂S(O)CH₃ | CF₃ | Ph-3-CH₃-4-F |
| H | CH(CH₃)CH₂S(O)CH₃ | CF₃ | Ph-3-CF₃-4-F |
| H | CH(CH₃)CH₂S(O)CH₃ | CF₃ | Ph-3-CF₃-4-Cl |
| H | CH(CH₃)CH₂S(O)CH₃ | CF₃ | Ph-2-F-4-CF₃ |
| H | CH(CH₃)CH₂S(O)CH₃ | CF₃ | Ph-3-F-4-CF₃ |
| H | CH(CH₃)CH₂S(O)CH₃ | CF₃ | Ph-2-Cl-4-CF₃ |
| H | CH(CH₃)CH₂S(O)CH₃ | CF₃ | Ph-3-F-4-OCHF₂ |
| H | CH(CH₃)CH₂S(O)CH₃ | CF₃ | Ph-3-Cl-4-OCHF₂ |
| H | CH(CH₃)CH₂S(O)CH₃ | CF₃ | Ph-3-Br-4-OCHF₂ |
| H | CH(CH₃)CH₂S(O)CH₃ | CF₃ | Ph-3-F-4-OCF₃ |
| H | CH(CH₃)CH₂S(O)CH₃ | CF₃ | Ph-3-Cl-4-OCF₃ |
| H | CH(CH₃)CH₂S(O)CH₃ | CF₃ | Ph-3-Br-4-OCF₃ |
| H | CH(CH₃)CH₂S(O)CH₃ | CF₃ | Ph-3-F-4-OCF₂Br |
| H | CH(CH₃)CH₂S(O)CH₃ | CF₃ | Ph-3-Cl-4-OCF₂Br |
| H | CH(CH₃)CH₂S(O)CH₃ | CF₃ | Ph-3-Br-4-OCF₂Br |
| H | CH(CH₃)CH₂S(O)CH₃ | CF₃ | Ph-3-F-4-OCF₂CHF₂ |
| H | CH(CH₃)CH₂S(O)CH₃ | CF₃ | Ph-3-Cl-4-OCF₂CHF₂ |
| H | CH(CH₃)CH₂S(O)CH₃ | CF₃ | Ph-3-Br-4-OCF₂CHF₂ |
| H | CH(CH₃)CH₂S(O)CH₃ | CF₃ | Ph-3-F-4-OCF₂CHFCl |
| H | CH(CH₃)CH₂S(O)CH₃ | CF₃ | Ph-3-Cl-4-OCF₂CHFCl |
| H | CH(CH₃)CH₂S(O)CH₃ | CF₃ | Ph-3-Br-4-OCf₂CHFCl |
| H | CH(CH₃)CH₂S(O)CH₃ | CF₃ | Ph-3-F-4-OCF₂CHFCF₃ |
| H | CH(CH₃)CH₂S(O)CH₃ | CF₃ | Ph-3-Cl-4-OCF₂CHFCF₃ |
| H | CH(CH₃)CH₂S(O)CH₃ | CF₃ | Ph-3-Br-4-OCF₂CHFCF₃ |
| H | CH(CH₃)CH₂S(O)CH₃ | CF₃ | Ph-3-F-4-OCF₂CHFOCF₃ |
| H | CH(CH₃)CH₂S(O)CH₃ | CF₃ | Ph-3-Cl-4-OCF₂CHFOCF₃ |
| H | CH(CH₃)CH₂S(O)CH₃ | CF₃ | Ph-3-Br-4-OCF₂CHFOCF₃ |
| H | CH(CH₃)CH₂S(O)CH₃ | CF₃ | Ph-3-CH₃-4-OCF₂CHFOCF₃ |
| H | CH(CH₃)CH₂S(O)CH₃ | CF₃ | Ph-3-F-4-OCF₂CHFOCF₂CF₂CF₃ |
| H | CH(CH₃)CH₂S(O)CH₃ | CF₃ | Ph-3-Cl-4-OCF₂CHFOCF₂CF₂CF₃ |
| H | CH(CH₃)CH₂S(O)CH₃ | CF₃ | Ph-3-Br-4-OCF₂CHFOCF₂CF₂CF₃ |
| H | CH(CH₃)CH₂S(O)CH₃ | CF₃ | Ph(-3-OCF₂O-4-) |
| H | CH(CH₃)CH₂S(O)CH₃ | CF₃ | Ph(-3-OCF₂CF₂O-4-) |
| H | CH(CH₃)CH₂S(O)CH₃ | CF₃ | Ph-3,4-F₂-5-CF₃ |
| H | CH(CH₃)CH₂S(O)CH₃ | CF₃ | 2-Naph |
| H | CH(CH₃)CH₂S(O)CH₃ | CF₃ | (L-1b)Br |
| H | CH(CH₃)CH₂S(O)CH₃ | CF₃ | (L-1c)Cl |
| H | CH(CH₃)CH₂S(O)CH₃ | CF₃ | (L-1c)Br |
| H | CH(CH₃)CH₂S(O)CH₃ | CF₃ | (L-1c)CF₃ |
| H | CH(CH₃)CH₂S(O)CH₃ | CF₃ | (L-2b)Br |
| H | CH(CH₃)CH₂S(O)CH₃ | CF₃ | (L-3b)Cl |
| H | CH(CH₃)CH₂S(O)CH₃ | CF₃ | (L-3b)Br |
| H | CH(CH₃)CH₂S(O)CH₃ | CF₃ | (L-3c)Cl |
| H | CH(CH₃)CH₂S(O)CH₃ | CF₃ | (L-3c)Br |
| H | CH(CH₃)CH₂S(O)CH₃ | CF₃ | (L-3c)CF₃ |
| H | CH(CH₃)CH₂S(O)CH₃ | CF₃ | L-3d |
| H | CH(CH₃)CH₂S(O)CH₃ | CF₃ | (L-4b)Cl |
| H | CH(CH₃)CH₂S(O)CH₃ | CF₃ | (L-4b)Br |
| H | CH(CH₃)CH₂S(O)CH₃ | CF₃ | (L-10b)Cl |
| H | CH(CH₃)CH₂S(O)CH₃ | CF₃ | (L-10b)Br |
| H | CH(CH₃)CH₂S(O)CH₃ | CF₃ | (L-15b)CF₃ |
| H | CH(CH₃)CH₂S(O)CH₃ | CF₃ | (L-16a)CHF₂ |
| H | CH(CH₃)CH₂S(O)CH₃ | CF₃ | (L-16a)CF₂Br |
| H | CH(CH₃)CH₂S(O)CH₃ | CF₃ | (L-17a)Cl |
| H | CH(CH₃)CH₂S(O)CH₃ | CF₃ | (L-21b)Cl |
| H | CH(CH₃)CH₂S(O)CH₃ | CF₃ | (L-21b)Br |
| H | CH(CH₃)CH₂S(O)CH₃ | CF₃ | (L-21b)CF₃ |
| H | CH(CH₃)CH₂S(O)CH₃ | CF₃ | (L-22b)Cl |
| H | CH(CH₃)CH₂S(O)CH₃ | CF₃ | (L-22b)Br |
| H | CH(CH₃)CH₂S(O)CH₃ | CF₃ | (L-23b)Cl |
| H | CH(CH₃)CH₂S(O)CH₃ | CF₃ | (L-23b)Br |
| H | CH(CH₃)CH₂S(O)CH₃ | CF₃ | (L-23c)Cl |
| H | CH(CH₃)CH₂S(O)CH₃ | CF₃ | (L-23c)Br |
| H | CH(CH₃)CH₂S(O)CH₃ | CF₃ | (L-31a)Cl |
| H | CH(CH₃)CH₂S(O)CH₃ | CF₃ | (L-31a)Br |
| H | CH(CH₃)CH₂S(O)CH₃ | CF₃ | (L-45c)F |
| H | CH(CH₃)CH₂S(O)CH₃ | CF₃ | (L-45c)Cl |
| H | CH(CH₃)CH₂S(O)CH₃ | CF₃ | (L-45c)Br |
| H | CH(CH₃)CH₂S(O)CH₃ | CF₃ | (L-45c)CF₃ |
| H | CH(CH₃)CH₂S(O)CH₃ | CF₃ | L-45e |
| H | CH(CH₃)CH₂S(O)CH₃ | CF₃ | L-45f |
| H | CH(CH₃)CH₂S(O)CH₃ | CF₃ | (L-46c)F |
| H | CH(CH₃)CH₂S(O)CH₃ | CF₃ | (L-46c)Cl |
| H | CH(CH₃)CH₂S(O)CH₃ | CF₃ | (L-46c)Br |
| H | CH(CH₃)CH₂S(O)CH₃ | CF₃ | (L-46c)CF₃ |
| H | CH(CH₃)CH₂S(O)CH₃ | CF₃ | L-46d |
| H | CH(CH₃)CH₂S(O)CH₃ | CF₃ | L-47a |
| H | CH(CH₃)CH₂S(O)CH₃ | CF₃ | L-47d |
| H | CH(CH₃)CH₂S(O)CH₃ | CF₃ | (L-48b)Br |
| H | CH(CH₃)CH₂S(O)CH₃ | CF₃ | (L-50b)Cl |
| H | CH(CH₃)CH₂S(O)CH₃ | CF₃ | (L-50b)Br |
| H | CH(CH₃)CH₂S(O)CH₃ | CF₃ | (L-51b)Cl |
| H | CH(CH₃)CH₂SO₂CH₃ | CH₃ | CF₂O(Ph-4-Cl) |
| H | CH(CH₃)CH₂SO₂CH₃ | CH₃ | CH═CH(Ph-4-Cl) |
| H | CH(CH₃)CH₂SO₂CH₃ | CH₃ | CH═CH(Ph-4-OCF₃) |
| H | CH(CH₃)CH₂SO₂CH₃ | CH₃ | CH═CH(Ph-3,4-Cl₂) |
| H | CH(CH₃)CH₂SO₂CH₃ | CH₃ | Ph-4-F |
| H | CH(CH₃)CH₂SO₂CH₃ | CH₃ | Ph-4-Cl |
| H | CH(CH₃)CH₂SO₂CH₃ | CH₃ | Ph-4-Br |
| H | CH(CH₃)CH₂SO₂CH₃ | CH₃ | Ph-4-I |
| H | CH(CH₃)CH₂SO₂CH₃ | CH₃ | Ph-4-CF₃ |
| H | CH(CH₃)CH₂SO₂CH₃ | CH₃ | Ph-4-OCHF₂ |
| H | CH(CH₃)CH₂SO₂CH₃ | CH₃ | Ph-4-OCF₃ |
| H | CH(CH₃)CH₂SO₂CH₃ | CH₃ | Ph-4-OCF₂Br |
| H | CH(CH₃)CH₂SO₂CH₃ | CH₃ | Ph-4-OCF₂CHF₂ |
| H | CH(CH₃)CH₂SO₂CH₃ | CH₃ | Ph-4-OCF₂CHFCl |
| H | CH(CH₃)CH₂SO₂CH₃ | CH₃ | Ph-4-OCF₂CHFCF₃ |
| H | CH(CH₃)CH₂SO₂CH₃ | CH₃ | Ph-4-OCF₂CHFOCF₃ |
| H | CH(CH₃)CH₂SO₂CH₃ | CH₃ | Ph-4-OCF₂CHFOCF₂CF₂CF₃ |
| H | CH(CH₃)CH₂SO₂CH₃ | CH₃ | Ph-4-O(L-45g) |
| H | CH(CH₃)CH₂SO₂CH₃ | CH₃ | Ph-3,4-Cl₂ |
| H | CH(CH₃)CH₂SO₂CH₃ | CH₃ | Ph(-3-OCF₂O-4-) |
| H | CH(CH₃)CH₂SO₂CH₃ | CH₃ | Ph(-3-OCF₂CF₂O-4-) |
| H | CH(CH₃)CH₂SO₂CH₃ | CH₃ | (L-45c)Cl |
| H | CH(CH₃)CH₂SO₂CH₃ | CH₃ | (L-45c)Br |
| H | CH(CH₃)CH₂SO₂CH₃ | CH₃ | (L-45c)CF₃ |
| H | CH(CH₃)CH₂SO₂CH₃ | CH₃ | L-45e |
| H | CH(CH₃)CH₂SO₂CH₃ | CH₃ | L-45f |
| H | CH(CH₃)CH₂SO₂CH₃ | CH₃ | (L-46c)Cl |
| H | CH(CH₃)CH₂SO₂CH₃ | CH₃ | (L-46c)Br |
| H | CH(CH₃)CH₂SO₂CH₃ | CH₃ | (L-46c)CF₃ |
| H | CH(CH₃)CH₂SO₂CH₃ | CH₃ | L-46d |
| H | CH(CH₃)CH₂SO₂CH₃ | Et | Ph-4-Cl |
| H | CH(CH₃)CH₂SO₂CH₃ | Et | Ph-4-Br |
| H | CH(CH₃)CH₂SO₂CH₃ | Et | Ph-4-CF₃ |
| H | CH(CH₃)CH₂SO₂CH₃ | Et | Ph-4-OCF₃ |
| H | CH(CH₃)CH₂SO₂CH₃ | Et | Ph-4-OCF₂Br |
| H | CH(CH₃)CH₂SO₂CH₃ | Et | Ph-4-OCF₂CHFCF₃ |
| H | CH(CH₃)CH₂SO₂CH₃ | Et | Ph-4-OCF₂CHFOCF₃ |
| H | CH(CH₃)CH₂SO₂CH₃ | Et | Ph-4-OCF₂CHFOCF₂CF₂CF₃ |
| H | CH(CH₃)CH₂SO₂CH₃ | Et | Ph-4-O(L-45g) |
| H | CH(CH₃)CH₂SO₂CH₃ | Et | Ph-3,4-Cl₂ |
| H | CH(CH₃)CH₂SO₂CH₃ | Et | Ph(-3-OCF₂O-4-) |
| H | CH(CH₃)CH₂SO₂CH₃ | Et | Ph(-3-OCF₂CF₂O-4-) |
| H | CH(CH₃)CH₂SO₂CH₃ | Et | (L-45c)CF₃ |
| H | CH(CH₃)CH₂SO₂CH₃ | n-Pr | Ph-4-Br |
| H | CH(CH₃)CH₂SO₂CH₃ | n-Pr | Ph-4-CF₃ |
| H | CH(CH₃)CH₂SO₂CH₃ | n-Pr | Ph-4-OCF₃ |
| H | CH(CH₃)CH₂SO₂CH₃ | n-Pr | (L-45c)CF₃ |
| H | CH(CH₃)CH₂SO₂CH₃ | i-Pr | Ph-4-Br |
| H | CH(CH₃)CH₂SO₂CH₃ | i-Pr | Ph-4-CF₃ |
| H | CH(CH₃)CH₂SO₂CH₃ | i-Pr | Ph-4-OCF₃ |
| H | CH(CH₃)CH₂SO₂CH₃ | i-Pr | (L-45c)CF₃ |
| H | CH(CH₃)CH₂SO₂CH₃ | c-Pr | Ph-4-Br |
| H | CH(CH₃)CH₂SO₂CH₃ | c-Pr | Ph-4-CF₃ |
| H | CH(CH₃)CH₂SO₂CH₃ | c-Pr | Ph-4-OCF₃ |
| H | CH(CH₃)CH₂SO₂CH₃ | c-Pr | (L-45c)CF₃ |
| H | CH(CH₃)CH₂SO₂CH₃ | n-Bu | (L-45c)CF₃ |
| H | CH(CH₃)CH₂SO₂CH₃ | CH₂F | Ph-4-Br |
| H | CH(CH₃)CH₂SO₂CH₃ | CH₂Cl | Ph-4-CF₃ |
| H | CH(CH₃)CH₂SO₂CH₃ | CH₂Br | Ph-4-OCF₃ |
| H | CH(CH₃)CH₂SO₂CH₃ | CHF₂ | Ph-4-Cl |
| H | CH(CH₃)CH₂SO₂CH₃ | CHF₂ | Ph-4-Br |
| H | CH(CH₃)CH₂SO₂CH₃ | CHF₂ | Ph-4-CF₃ |
| H | CH(CH₃)CH₂SO₂CH₃ | CHF₂ | Ph-4-OCF₃ |
| H | CH(CH₃)CH₂SO₂CH₃ | CHF₂ | Ph-4-OCF₂Br |
| H | CH(CH₃)CH₂SO₂CH₃ | CHF₂ | Ph-4-OCF₂CHFCF₃ |
| H | CH(CH₃)CH₂SO₂CH₃ | CHF₂ | Ph-4-OCF₂CHFOCF₃ |
| H | CH(CH₃)CH₂SO₂CH₃ | CHF₂ | Ph-4-OCF₂CHFOCF₂CF₂CF₃ |
| H | CH(CH₃)CH₂SO₂CH₃ | CHF₂ | Ph-4-O(L-45g) |
| H | CH(CH₃)CH₂SO₂CH₃ | CHF₂ | Ph-3,4-Cl₂ |
| H | CH(CH₃)CH₂SO₂CH₃ | CHF₂ | Ph(-3-OCF₂O-4-) |
| H | CH(CH₃)CH₂SO₂CH₃ | CHF₂ | Ph(-3-OCF₂CF₂O-4-) |
| H | CH(CH₃)CH₂SO₂CH₃ | CHF₂ | (L-45c)CF₃ |
| H | CH(CH₃)CH₂SO₂CH₃ | CHF₂ | L-45f |
| H | CH(CH₃)CH₂SO₂CH₃ | CHF₂ | (L-46c)CF₃ |
| H | CH(CH₃)CH₂SO₂CH₃ | CHF₂ | L-46d |
| H | CH(CH₃)CH₂SO₂CH₃ | CHFCl | (L-45c)CF₃ |
| H | CH(CH₃)CH₂SO₂CH₃ | CHFBr | Ph-4-Br |
| H | CH(CH₃)CH₂SO₂CH₃ | CF₃ | c-Pr |
| H | CH(CH₃)CH₂SO₂CH₃ | CF₃ | c-Bu |
| H | CH(CH₃)CH₂SO₂CH₃ | CF₃ | c-Pen |
| H | CH(CH₃)CH₂SO₂CH₃ | CF₃ | c-Hex |
| H | CH(CH₃)CH₂SO₂CH₃ | CF₃ | T-1 |
| H | CH(CH₃)CH₂SO₂CH₃ | CF₃ | T-2 |
| H | CH(CH₃)CH₂SO₂CH₃ | CF₃ | T-3 |
| H | CH(CH₃)CH₂SO₂CH₃ | CF₃ | T-4 |
| H | CH(CH₃)CH₂SO₂CH₃ | CF₃ | T-5 |
| H | CH(CH₃)CH₂SO₂CH₃ | CF₃ | CH₂OCH₃ |
| H | CH(CH₃)CH₂SO₂CH₃ | CF₃ | CH₂OEt |
| H | CH(CH₃)CH₂SO₂CH₃ | CF₃ | CH₂OPr-n |
| H | CH(CH₃)CH₂SO₂CH₃ | CF₃ | CH₂OPr-i |
| H | CH(CH₃)CH₂SO₂CH₃ | CF₃ | CH₂OBu-n |
| H | CH(CH₃)CH₂SO₂CH₃ | CF₃ | CH₂OC(O)(Ph-2-Cl) |
| H | CH(CH₃)CH₂SO₂CH₃ | CF₃ | CH₂OC(O)(Ph-3-Cl) |
| H | CH(CH₃)CH₂SO₂CH₃ | CF₃ | CH₂OC(O)(Ph-4-Cl) |
| H | CH(CH₃)CH₂SO₂CH₃ | CF₃ | CH₂OPh |
| H | CH(CH₃)CH₂SO₂CH₃ | CF₃ | CH₂O(Ph-2-F) |
| H | CH(CH₃)CH₂SO₂CH₃ | CF₃ | CH₂O(Ph-3-F) |
| H | CH(CH₃)CH₂SO₂CH₃ | CF₃ | CH₂O(Ph-4-F) |
| H | CH(CH₃)CH₂SO₂CH₃ | CF₃ | CH₂O(Ph-2-Cl) |
| H | CH(CH₃)CH₂SO₂CH₃ | CF₃ | CH₂O(Ph-3-Cl) |
| H | CH(CH₃)CH₂SO₂CH₃ | CF₃ | CH₂O(Ph-4-Cl) |
| H | CH(CH₃)CH₂SO₂CH₃ | CF₃ | CH₂O(Ph-2-Br) |
| H | CH(CH₃)CH₂SO₂CH₃ | CF₃ | CH₂O(Ph-3-Br) |
| H | CH(CH₃)CH₂SO₂CH₃ | CF₃ | CH₂O(Ph-4-Br) |
| H | CH(CH₃)CH₂SO₂CH₃ | CF₃ | CH₂O(Ph-2-CF₃) |
| H | CH(CH₃)CH₂SO₂CH₃ | CF₃ | CH₃O(Ph-3-CF₃) |
| H | CH(CH₃)CH₂SO₂CH₃ | CF₃ | CH₂O(Ph-4-CF₃) |
| H | CH(CH₃)CH₂SO₂CH₃ | CF₃ | CH₂O(Ph-2-OCF₃) |
| H | CH(CH₃)CH₂SO₂CH₃ | CF₃ | CH₂O(Ph-3-OCF₃) |
| H | CH(CH₃)CH₂SO₂CH₃ | CF₃ | CH₂O(Ph-4-OCF₃) |
| H | CH(CH₃)CH₂SO₂CH₃ | CF₃ | CF₂OPh |
| H | CH(CH₃)CH₂SO₂CH₃ | CF₃ | CF₂O(Ph-2-F) |
| H | CH(CH₃)CH₂SO₂CH₃ | CF₃ | CF₂O(Ph-3-F) |
| H | CH(CH₃)CH₂SO₂CH₃ | CF₃ | CF₂O(Ph-4-F) |
| H | CH(CH₃)CH₂SO₂CH₃ | CF₃ | CF₂O(Ph-2-Cl) |
| H | CH(CH₃)CH₂SO₂CH₃ | CF₃ | CF₂O(Ph-3-Cl) |
| H | CH(CH₃)CH₂SO₂CH₃ | CF₃ | CF₂O(Ph-4-Cl) |
| H | CH(CH₃)CH₂SO₂CH₃ | CF₃ | CF₂O(Ph-2-Br) |
| H | CH(CH₃)CH₂SO₂CH₃ | CF₃ | CF₂O(Ph-3-Br) |
| H | CH(CH₃)CH₂SO₂CH₃ | CF₃ | CF₂O(Ph-4-Br) |
| H | CH(CH₃)CH₂SO₂CH₃ | CF₃ | CF₂O(Ph-2-CF₃) |
| H | CH(CH₃)CH₂SO₂CH₃ | CF₃ | CF₂O(Ph-3-CF₃) |
| H | CH(CH₃)CH₂SO₂CH₃ | CF₃ | CF₂O(Ph-4-CF₃) |
| H | CH(CH₃)CH₂SO₂CH₃ | CF₃ | CF₂O(Ph-2-OCF₃) |
| H | CH(CH₃)CH₂SO₂CH₃ | CF₃ | CF₂O(Ph-3-OCF₃) |
| H | CH(CH₃)CH₂SO₂CH₃ | CF₃ | CF₂O(Ph-4-OCF₃) |
| H | CH(CH₃)CH₂SO₂CH₃ | CF₃ | CH₂S(Ph-2-Cl) |
| H | CH(CH₃)CH₂SO₂CH₃ | CF₃ | CH₂S(Ph-3-Cl) |
| H | CH(CH₃)CH₂SO₂CH₃ | CF₃ | CH₂S(Ph-4-Cl) |
| H | CH(CH₃)CH₂SO₂CH₃ | CF₃ | CH₂SO₂(Ph-2-Cl) |
| H | CH(CH₃)CH₂SO₂CH₃ | CF₃ | CH₂SO₂(Ph-3-Cl) |
| H | CH(CH₃)CH₂SO₂CH₃ | CF₃ | CH₂SO₂(Ph-4-Cl) |
| H | CH(CH₃)CH₂SO₂CH₃ | CF₃ | CH₂N(CH₃)₂ |
| H | CH(CH₃)CH₂SO₂CH₃ | CF₃ | CH₂NH(Ph-2-Cl) |
| H | CH(CH₃)CH₂SO₂CH₃ | CF₃ | CH₂NH(Ph-3-Cl) |
| H | CH(CH₃)CH₂SO₂CH₃ | CF₃ | CH₂NH(Ph-4-Cl) |
| H | CH(CH₃)CH₂SO₂CH₃ | CF₃ | CF₂C(O)OEt |
| H | CH(CH₃)CH₂SO₂CH₃ | CF₃ | CH₂(L-5a) |
| H | CH(CH₃)CH₂SO₂CH₃ | CF₃ | CH₂(L-14a) |
| H | CH(CH₃)CH₂SO₂CH₃ | CF₃ | CH₂(L-24a) |
| H | CH(CH₃)CH₂SO₂CH₃ | CF₃ | CH₂(L-36a) |
| H | CH(CH₃)CH₂SO₂CH₃ | CF₃ | CH₂CH₂Ph |
| H | CH(CH₃)CH₂SO₂CH₃ | CF₃ | CH₂CH₂(Ph-2-Cl) |
| H | CH(CH₃)CH₂SO₂CH₃ | CF₃ | CH₂CH₂(Ph-3-Cl) |
| H | CH(CH₃)CH₂SO₂CH₃ | CF₃ | CH₂CH₂(Ph-4-Cl) |
| H | CH(CH₃)CH₂SO₂CH₃ | CF₃ | T-22 |
| H | CH(CH₃)CH₂SO₂CH₃ | CF₃ | T-23 |
| H | CH(CH₃)CH₂SO₂CH₃ | CF₃ | T-24 |
| H | CH(CH₃)CH₂SO₂CH₃ | CF₃ | C(O)OEt |
| H | CH(CH₃)CH₂SO₂CH₃ | CF₃ | C(O)OBu-t |
| H | CH(CH₃)CH₂SO₂CH₃ | CF₃ | C(O)OCH₂CF₃ |
| H | CH(CH₃)CH₂SO₂CH₃ | CF₃ | CH═CH(Ph-2-F) |
| H | CH(CH₃)CH₂SO₂CH₃ | CF₃ | CH═CH(Ph-3-F) |
| H | CH(CH₃)CH₂SO₂CH₃ | CF₃ | CH═CH(Ph-4-F) |
| H | CH(CH₃)CH₂SO₂CH₃ | CF₃ | CH═CH(Ph-2-Cl) |
| H | CH(CH₃)CH₂SO₂CH₃ | CF₃ | CH═CH(Ph-3-Cl) |
| H | CH(CH₃)CH₂SO₂CH₃ | CF₃ | CH═CH(Ph-4-Cl) |
| H | CH(CH₃)CH₂SO₂CH₃ | CF₃ | CH═CH(Ph-2-Br) |
| H | CH(CH₃)CH₂SO₂CH₃ | CF₃ | CH═CH(Ph-3-Br) |
| H | CH(CH₃)CH₂SO₂CH₃ | CF₃ | CH═CH(Ph-4-Br) |
| H | CH(CH₃)CH₂SO₂CH₃ | CF₃ | CH═CH(Ph-2-CF₃) |
| H | CH(CH₃)CH₂SO₂CH₃ | CF₃ | CH═CH(Ph-3-CF₃) |
| H | CH(CH₃)CH₂SO₂CH₃ | CF₃ | CH═CH(Ph-4-CF₃) |
| H | CH(CH₃)CH₂SO₂CH₃ | CF₃ | CH═CH(Ph-2-OCF₃) |
| H | CH(CH₃)CH₂SO₂CH₃ | CF₃ | CH═CH(Ph-3-OCF₃) |
| H | CH(CH₃)CH₂SO₂CH₃ | CF₃ | CH═CH(Ph-4-OCF₃) |
| H | CH(CH₃)CH₂SO₂CH₃ | CF₃ | CH═CH(Ph-2-SCH₃) |
| H | CH(CH₃)CH₂SO₂CH₃ | CF₃ | CH═CH(Ph-3-SCH₃) |
| H | CH(CH₃)CH₂SO₂CH₃ | CF₃ | CH═CH(Ph-4-SCH₃) |
| H | CH(CH₃)CH₂SO₂CH₃ | CF₃ | CH═CH(Ph-2-SO₂CH₃) |
| H | CH(CH₃)CH₂SO₂CH₃ | CF₃ | CH═CH(Ph-3-SO₂CH₃) |
| H | CH(CH₃)CH₂SO₂CH₃ | CF₃ | CH═CH(Ph-4-SO₂CH₃) |
| H | CH(CH₃)CH₂SO₂CH₃ | CF₃ | CH═CH(Ph-3,4-F₂) |
| H | CH(CH₃)CH₂SO₂CH₃ | CF₃ | CH═CH(Ph-3-F-4-Cl) |
| H | CH(CH₃)CH₂SO₂CH₃ | CF₃ | CH═CH(Ph-3,4-Cl₂) |
| H | CH(CH₃)CH₂SO₂CH₃ | CF₃ | CH═CH(Ph-3,4-Br₂) |
| H | CH(CH₃)CH₂SO₂CH₃ | CF₃ | CH═CH(Ph-3-F-4-CF₃) |
| H | CH(CH₃)CH₂SO₂CH₃ | CF₃ | CH═CH(Ph-3-Cl-4-OCF₃) |
| H | CH(CH₃)CH₂SO₂CH₃ | CF₃ | CH═CH[Ph(-3-OCF₂O-4-)] |
| H | CH(CH₃)CH₂SO₂CH₃ | CF₃ | Ph |
| H | CH(CH₃)CH₂SO₂CH₃ | CF₃ | Ph-2-F |
| H | CH(CH₃)CH₂SO₂CH₃ | CF₃ | Ph-3-F |
| H | CH(CH₃)CH₂SO₂CH₃ | CF₃ | Ph-4-F |
| H | CH(CH₃)CH₂SO₂CH₃ | CF₃ | Ph-2-Cl |
| H | CH(CH₃)CH₂SO₂CH₃ | CF₃ | Ph-3-Cl |
| H | CH(CH₃)CH₂SO₂CH₃ | CF₃ | Ph-4-Cl |
| H | CH(CH₃)CH₂SO₂CH₃ | CF₃ | Ph-2-Br |
| H | CH(CH₃)CH₂SO₂CH₃ | CF₃ | Ph-3-Br |
| H | CH(CH₃)CH₂SO₂CH₃ | CF₃ | Ph-4-Br |
| H | CH(CH₃)CH₂SO₂CH₃ | CF₃ | Ph-4-I |
| H | CH(CH₃)CH₂SO₂CH₃ | CF₃ | Ph-4-Bu-t |
| H | CH(CH₃)CH₂SO₂CH₃ | CF₃ | Ph-2-CF₃ |
| H | CH(CH₃)CH₂SO₂CH₃ | CF₃ | Ph-3-CF₃ |
| H | CH(CH₃)CH₂SO₂CH₃ | CF₃ | Ph-4-CF₃ |
| H | CH(CH₃)CH₂SO₂CH₃ | CF₃ | Ph-4-OCH₃ |
| H | CH(CH₃)CH₂SO₂CH₃ | CF₃ | Ph-4-OCHF₂ |
| H | CH(CH₃)CH₂SO₂CH₃ | CF₃ | Ph-2-OCF₃ |
| H | CH(CH₃)CH₂SO₂CH₃ | CF₃ | Ph-3-OCF₃ |
| H | CH(CH₃)CH₂SO₂CH₃ | CF₃ | Ph-4-OCF₃ |
| H | CH(CH₃)CH₂SO₂CH₃ | CF₃ | Ph-4-OCF₂Br |
| H | CH(CH₃)CH₂SO₂CH₃ | CF₃ | Ph-4-OCH₂CF₃ |
| H | CH(CH₃)CH₂SO₂CH₃ | CF₃ | Ph-4-OCF₂CHF₂ |
| H | CH(CH₃)CH₂SO₂CH₃ | CF₃ | Ph-4-OCF₂CHFCl |
| H | CH(CH₃)CH₂SO₂CH₃ | CF₃ | Ph-4-OCF₂CHFBr |
| H | CH(CH₃)CH₂SO₂CH₃ | CF₃ | Ph-4-OCF₂CF₂Br |
| H | CH(CH₃)CH₂SO₂CH₃ | CF₃ | Ph-4-OCF₂CFCl₂ |
| H | CH(CH₃)CH₂SO₂CH₃ | CF₃ | Ph-4-OCF₂CCl₃ |
| H | CH(CH₃)CH₂SO₂CH₃ | CF₃ | Ph-4-OCH₂CF₂CHF₂ |
| H | CH(CH₃)CH₂SO₂CH₃ | CF₃ | Ph-3-OCF₂CHFCF₃ |
| H | CH(CH₃)CH₂SO₂CH₃ | CF₃ | Ph-4-OCF₂CHFCF₃ |
| H | CH(CH₃)CH₂SO₂CH₃ | CF₃ | Ph-4-OCH(CF₃)₂ |
| H | CH(CH₃)CH₂SO₂CH₃ | CF₃ | Ph-4-OCF₂CFBrCF₃ |
| H | CH(CH₃)CH₂SO₂CH₃ | CF₃ | Ph-3-OCF₂CHFOCF₃ |
| H | CH(CH₃)CH₂SO₂CH₃ | CF₃ | Ph-4-OCF₂CHFOCF₃ |
| H | CH(CH₃)CH₂SO₂CH₃ | CF₃ | Ph-4-OCF₂CHFOCF₂CF₂CF₃ |
| H | CH(CH₃)CH₂SO₂CH₃ | CF₃ | Ph-4-OSO₂CH₃ |
| H | CH(CH₃)CH₂SO₂CH₃ | CF₃ | Ph-4-OSO₂CF₃ |
| H | CH(CH₃)CH₂SO₂CH₃ | CF₃ | Ph-3-O(Ph-4-Cl) |
| H | CH(CH₃)CH₂SO₂CH₃ | CF₃ | Ph-4-O(Ph-4-Cl) |
| H | CH(CH₃)CH₂SO₂CH₃ | CF₃ | Ph-4-O(Ph-4-Br) |
| H | CH(CH₃)CH₂SO₂CH₃ | CF₃ | Ph-4-O(Ph-4-CF₃) |
| H | CH(CH₃)CH₂SO₂CH₃ | CF₃ | Ph-4-O(L-45c)Br |
| H | CH(CH₃)CH₂SO₂CH₃ | CF₃ | Ph-4-O(L-45c)CF₃ |
| H | CH(CH₃)CH₂SO₂CH₃ | CF₃ | Ph-3-O(L-45e) |
| H | CH(CH₃)CH₂SO₂CH₃ | CF₃ | Ph-4-O(L-45e) |
| H | CH(CH₃)CH₂SO₂CH₃ | CF₃ | Ph-4-O(L-48b)Br |
| H | CH(CH₃)CH₂SO₂CH₃ | CF₃ | Ph-4-SCH₃ |
| H | CH(CH₃)CH₂SO₂CH₃ | CF₃ | Ph-4-SO₂CH₃ |
| H | CH(CH₃)CH₂SO₂CH₃ | CF₃ | Ph-3-S(Ph-4-Cl) |
| H | CH(CH₃)CH₂SO₂CH₃ | CF₃ | Ph-4-S(Ph-4-Cl) |
| H | CH(CH₃)CH₂SO₂CH₃ | CF₃ | Ph-4-S(Ph-4-Br) |
| H | CH(CH₃)CH₂SO₂CH₃ | CF₃ | Ph-4-S(Ph-4-CF₃) |
| H | CH(CH₃)CH₂SO₂CH₃ | CF₃ | Ph-4-S(L-45c)Br |
| H | CH(CH₃)CH₂SO₂CH₃ | CF₃ | Ph-4-S(L-45c)CF₃ |
| H | CH(CH₃)CH₂SO₂CH₃ | CF₃ | Ph-4-S(L-45e) |
| H | CH(CH₃)CH₂SO₂CH₃ | CF₃ | Ph-4-S(L-48b)Br |
| H | CH(CH₃)CH₂SO₂CH₃ | CF₃ | Ph-4-NO₂ |
| H | CH(CH₃)CH₂SO₂CH₃ | CF₃ | Ph-4-CN |
| H | CH(CH₃)CH₂SO₂CH₃ | CF₃ | Ph-2,3-F₂ |
| H | CH(CH₃)CH₂SO₂CH₃ | CF₃ | Ph-2,4-F₂ |
| H | CH(CH₃)CH₂SO₂CH₃ | CF₃ | Ph-3,4-F₂ |
| H | CH(CH₃)CH₂SO₂CH₃ | CF₃ | Ph-2,5-F₂ |
| H | CH(CH₃)CH₂SO₂CH₃ | CF₃ | Ph-3,5-F₂ |
| H | CH(CH₃)CH₂SO₂CH₃ | CF₃ | Ph-2-Cl-4-F |
| H | CH(CH₃)CH₂SO₂CH₃ | CF₃ | Ph-2-F-3-Cl |
| H | CH(CH₃)CH₂SO₂CH₃ | CF₃ | Ph-3-Cl-4-F |
| H | CH(CH₃)CH₂SO₂CH₃ | CF₃ | Ph-2-F-4-Cl |
| H | CH(CH₃)CH₂SO₂CH₃ | CF₃ | Ph-3-F-4-Cl |
| H | CH(CH₃)CH₂SO₂CH₃ | CF₃ | Ph-2,3-Cl₂ |
| H | CH(CH₃)CH₂SO₂CH₃ | CF₃ | Ph-2,4-Cl₂ |
| H | CH(CH₃)CH₂SO₂CH₃ | CF₃ | Ph-2,5-Cl₂ |
| H | CH(CH₃)CH₂SO₂CH₃ | CF₃ | Ph-3,4-Cl₂ |
| H | CH(CH₃)CH₂SO₂CH₃ | CF₃ | Ph-3,5-Cl₂ |
| H | CH(CH₃)CH₂SO₂CH₃ | CF₃ | Ph-3-Br-4-F |
| H | CH(CH₃)CH₂SO₂CH₃ | CF₃ | Ph-2-F-4-Br |
| H | CH(CH₃)CH₂SO₂CH₃ | CF₃ | Ph-2-F-5-Br |
| H | CH(CH₃)CH₂SO₂CH₃ | CF₃ | Ph-3,4-Br₂ |
| H | CH(CH₃)CH₂SO₂CH₃ | CF₃ | Ph-3,5-Br₂ |
| H | CH(CH₃)CH₂SO₂CH₃ | CF₃ | Ph-3-CH₃-4-F |
| H | CH(CH₃)CH₂SO₂CH₃ | CF₃ | Ph-3-F-4-CH₃ |
| H | CH(CH₃)CH₂SO₂CH₃ | CF₃ | Ph-2-F-5-CH₃ |
| H | CH(CH₃)CH₂SO₂CH₃ | CF₃ | Ph-2,4-(CH₃)₂ |
| H | CH(CH₃)CH₂SO₂CH₃ | CF₃ | Ph-3,4-(CH₃)₂ |
| H | CH(CH₃)CH₂SO₂CH₃ | CF₃ | Ph-2-F-3-CF₃ |
| H | CH(CH₃)CH₂SO₂CH₃ | CF₃ | Ph-3-CF₃-4-F |
| H | CH(CH₃)CH₂SO₂CH₃ | CF₃ | Ph-3-CF₃-4-Cl |
| H | CH(CH₃)CH₂SO₂CH₃ | CF₃ | Ph-2-F-4-CF₃ |
| H | CH(CH₃)CH₂SO₂CH₃ | CF₃ | Ph-3-F-4-CF₃ |
| H | CH(CH₃)CH₂SO₂CH₃ | CF₃ | Ph-2-F-5-CF₃ |
| H | CH(CH₃)CH₂SO₂CH₃ | CF₃ | Ph-3-F-5-CF₃ |
| H | CH(CH₃)CH₂SO₂CH₃ | CF₃ | Ph-2-Cl-4-CF₃ |
| H | CH(CH₃)CH₂SO₂CH₃ | CF₃ | Ph-3,5-(CF₃)₂ |
| H | CH(CH₃)CH₂SO₂CH₃ | CF₃ | Ph-3-Br-4-OCH₃ |
| H | CH(CH₃)CH₂SO₂CH₃ | CF₃ | Ph-3-F-4-OCHF₂ |
| H | CH(CH₃)CH₂SO₂CH₃ | CF₃ | Ph-3-Cl-4-OCHF₂ |
| H | CH(CH₃)CH₂SO₂CH₃ | CF₃ | Ph-3-Br-4-OCHF₂ |
| H | CH(CH₃)CH₂SO₂CH₃ | CF₃ | Ph-3-F-4-OCF₃ |
| H | CH(CH₃)CH₂SO₂CH₃ | CF₃ | Ph-3-Cl-4-OCF₃ |
| H | CH(CH₃)CH₂SO₂CH₃ | CF₃ | Ph-3-Br-4-OCF₃ |
| H | CH(CH₃)CH₂SO₂CH₃ | CF₃ | Ph-3-F-4-OCF₂Br |
| H | CH(CH₃)CH₂SO₂CH₃ | CF₃ | Ph-3-Cl-4-OCF₂Br |
| H | CH(CH₃)CH₂SO₂CH₃ | CF₃ | Ph-3-Br-4-OCF₂Br |
| H | CH(CH₃)CH₂SO₂CH₃ | CF₃ | Ph-3-F-4-OCF₂CHF₂ |
| H | CH(CH₃)CH₂SO₂CH₃ | CF₃ | Ph-3-Cl-4-OCF₂CHF₂ |
| H | CH(CH₃)CH₂SO₂CH₃ | CF₃ | Ph-3-Br-4-OCF₂CHF₂ |
| H | CH(CH₃)CH₂SO₂CH₃ | CF₃ | Ph-3-F-4-OCF₂CHFCl |
| H | CH(CH₃)CH₂SO₂CH₃ | CF₃ | Ph-3-Cl-4-OCf₂CHFCl |
| H | CH(CH₃)CH₂SO₂CH₃ | CF₃ | Ph-3-Br-4-OCF₂CHFCl |
| H | CH(CH₃)CH₂SO₂CH₃ | CF₃ | Ph-3-F-4-OCF₂CHFCF₃ |
| H | CH(CH₃)CH₂SO₂CH₃ | CF₃ | Ph-3-Cl-4-OCF₂CHFCF₃ |
| H | CH(CH₃)CH₂SO₂CH₃ | CF₃ | Ph-3-Br-4-OCF₂CHFCF₃ |
| H | CH(CH₃)CH₂SO₂CH₃ | CF₃ | Ph-3-F-4-OCF₂CHFOCF₃ |
| H | CH(CH₃)CH₂SO₂CH₃ | CF₃ | Ph-3-Cl-4-OCF₂CHFOCF₃ |
| H | CH(CH₃)CH₂SO₂CH₃ | CF₃ | Ph-3-Br-4-OCF₂CHFOCF₃ |
| H | CH(CH₃)CH₂SO₂CH₃ | CF₃ | Ph-3-CH₃-4-OCF₂CHFOCF₃ |
| H | CH(CH₃)CH₂SO₂CH₃ | CF₃ | Ph-3-F-4-OCF₂CHFOCF₂CF₂CF₃ |
| H | CH(CH₃)CH₂SO₂CH₃ | CF₃ | Ph-3-Cl-4-OCF₂CHFOCF₂CF₂CF₃ |
| H | CH(CH₃)CH₂SO₂CH₃ | CF₃ | Ph-3-Br-4-OCF₂CHFOCF₂CF₂CF₃ |
| H | CH(CH₃)CH₂SO₂CH₃ | CF₃ | Ph(-3-OCF₂O-4-) |
| H | CH(CH₃)CH₂SO₂CH₃ | CF₃ | Ph(-3-OCF₂CF₂O-4-) |
| H | CH(CH₃)CH₂SO₂CH₃ | CF₃ | Ph-3-OPh-4-F |
| H | CH(CH₃)CH₂SO₂CH₃ | CF₃ | Ph-3-NO₂-4-F |
| H | CH(CH₃)CH₂SO₂CH₃ | CF₃ | Ph-3-NO₂-4-Cl |
| H | CH(CH₃)CH₂SO₂CH₃ | CF₃ | Ph-2-F-5-NO₂ |
| H | CH(CH₃)CH₂SO₂CH₃ | CF₃ | Ph-3-CN-4-F |
| H | CH(CH₃)CH₂SO₂CH₃ | CF₃ | Ph-2,3,4-F₃ |
| H | CH(CH₃)CH₂SO₂CH₃ | CF₃ | Ph-2,3,5-F₃ |
| H | CH(CH₃)CH₂SO₂CH₃ | CF₃ | Ph-2,4,5-F₃ |
| H | CH(CH₃)CH₂SO₂CH₃ | CF₃ | Ph-3,4,5-F₃ |
| H | CH(CH₃)CH₂SO₂CH₃ | CF₃ | Ph-2,3-F₂-4-CH₃ |
| H | CH(CH₃)CH₂SO₂CH₃ | CF₃ | Ph-2,3-F₂-4-CF₃ |
| H | CH(CH₃)CH₂SO₂CH₃ | CF₃ | Ph-3,4-F₂-5-CF₃ |
| H | CH(CH₃)CH₂SO₂CH₃ | CF₃ | Ph-2-F-3-Cl-5-CF₃ |
| H | CH(CH₃)CH₂SO₂CH₃ | CF₃ | Ph-3,5-Cl₂-4-OCH₃ |
| H | CH(CH₃)CH₂SO₂CH₃ | CF₃ | 1-Naph |
| H | CH(CH₃)CH₂SO₂CH₃ | CF₃ | 2-Naph |
| H | CH(CH₃)CH₂SO₂CH₃ | CF₃ | (L-1b)Br |
| H | CH(CH₃)CH₂SO₂CH₃ | CF₃ | (L-1c)Cl |
| H | CH(CH₃)CH₂SO₂CH₃ | CF₃ | (L-1c)Br |
| H | CH(CH₃)CH₂SO₂CH₃ | CF₃ | (L-1c)I |
| H | CH(CH₃)CH₂SO₂CH₃ | CF₃ | (L-1c)CF₃ |
| H | CH(CH₃)CH₂SO₂CH₃ | CF₃ | (L-2b)Br |
| H | CH(CH₃)CH₂SO₂CH₃ | CF₃ | (L-3b)Cl |
| H | CH(CH₃)CH₂SO₂CH₃ | CF₃ | (L-3b)Br |
| H | CH(CH₃)CH₂SO₂CH₃ | CF₃ | (L-3c)F |
| H | CH(CH₃)CH₂SO₂CH₃ | CF₃ | (L-3c)Cl |
| H | CH(CH₃)CH₂SO₂CH₃ | CF₃ | (L-3c)Br |
| H | CH(CH₃)CH₂SO₂CH₃ | CF₃ | (L-3c)I |
| H | CH(CH₃)CH₂SO₂CH₃ | CF₃ | (L-3c)CF₃ |
| H | CH(CH₃)CH₂SO₂CH₃ | CF₃ | (L-3c)CN |
| H | CH(CH₃)CH₂SO₂CH₃ | CF₃ | L-3d |
| H | CH(CH₃)CH₂SO₂CH₃ | CF₃ | (L-4b)Cl |
| H | CH(CH₃)CH₂SO₂CH₃ | CF₃ | (L-4b)Br |
| H | CH(CH₃)CH₂SO₂CH₃ | CF₃ | (L-4b)CN |
| H | CH(CH₃)CH₂SO₂CH₃ | CF₃ | (L-10b)Cl |
| H | CH(CH₃)CH₂SO₂CH₃ | CF₃ | (L-10b)Br |
| H | CH(CH₃)CH₂SO₂CH₃ | CF₃ | (L-15b)CF₃ |
| H | CH(CH₃)CH₂SO₂CH₃ | CF₃ | (L-16a)CHF₂ |
| H | CH(CH₃)CH₂SO₂CH₃ | CF₃ | (L-16a)CF₂Br |
| H | CH(CH₃)CH₂SO₂CH₃ | CF₃ | (L-17a)Cl |
| H | CH(CH₃)CH₂SO₂CH₃ | CF₃ | (L-21b)Cl |
| H | CH(CH₃)CH₂SO₂CH₃ | CF₃ | (L-21b)Br |
| H | CH(CH₃)CH₂SO₂CH₃ | CF₃ | (L-21b)I |
| H | CH(CH₃)CH₂SO₂CH₃ | CF₃ | (L-21b)CF₃ |
| H | CH(CH₃)CH₂SO₂CH₃ | CF₃ | (L-22b)Cl |
| H | CH(CH₃)CH₂SO₂CH₃ | CF₃ | (L-22b)Br |
| H | CH(CH₃)CH₂SO₂CH₃ | CF₃ | (L-23b)Cl |
| H | CH(CH₃)CH₂SO₂CH₃ | CF₃ | (L-23b)Br |
| H | CH(CH₃)CH₂SO₂CH₃ | CF₃ | (L-23c)Cl |
| H | CH(CH₃)CH₂SO₂CH₃ | CF₃ | (L-23c)Br |
| H | CH(CH₃)CH₂SO₂CH₃ | CF₃ | (L-31a)Cl |
| H | CH(CH₃)CH₂SO₂CH₃ | CF₃ | (L-31a)Br |
| H | CH(CH₃)CH₂SO₂CH₃ | CF₃ | (L-45c)F |
| H | CH(CH₃)CH₂SO₂CH₃ | CF₃ | (L-45c)Cl |
| H | CH(CH₃)CH₂SO₂CH₃ | CF₃ | (L-45c)Br |
| H | CH(CH₃)CH₂SO₂CH₃ | CF₃ | (L-45c)I |
| H | CH(CH₃)CH₂SO₂CH₃ | CF₃ | (L-45c)CF₃ |
| H | CH(CH₃)CH₂SO₂CH₃ | CF₃ | L-45e |
| H | CH(CH₃)CH₂SO₂CH₃ | CF₃ | L-45f |
| H | CH(CH₃)CH₂SO₂CH₃ | CF₃ | (L-46c)F |
| H | CH(CH₃)CH₂SO₂CH₃ | CF₃ | (L-46c)Cl |
| H | CH(CH₃)CH₂SO₂CH₃ | CF₃ | (L-46c)Br |
| H | CH(CH₃)CH₂SO₂CH₃ | CF₃ | (L-46c)I |
| H | CH(CH₃)CH₂SO₂CH₃ | CF₃ | (L-46c)CF₃ |
| H | CH(CH₃)CH₂SO₂CH₃ | CF₃ | (L-46c)OCH₂CF₃ |
| H | CH(CH₃)CH₂SO₂CH₃ | CF₃ | (L-46c)OCH(CF₃)₂ |
| H | CH(CH₃)CH₂SO₂CH₃ | CF₃ | L-46d |
| H | CH(CH₃)CH₂SO₂CH₃ | CF₃ | L-47a |
| H | CH(CH₃)CH₂SO₂CH₃ | CF₃ | L-47d |
| H | CH(CH₃)CH₂SO₂CH₃ | CF₃ | (L-48b)Br |
| H | CH(CH₃)CH₂SO₂CH₃ | CF₃ | (L-50b)Cl |
| H | CH(CH₃)CH₂SO₂CH₃ | CF₃ | (L-50b)Br |
| H | CH(CH₃)CH₂SO₂CH₃ | CF₃ | (L-51b)Cl |
| H | CH(CH₃)CH₂SO₂CH₃ | CF₂Cl | Ph-4-Cl |
| H | CH(CH₃)CH₂SO₂CH₃ | CF₂Cl | Ph-4-Br |
| H | CH(CH₃)CH₂SO₂CH₃ | CF₂Cl | Ph-4-CF₃ |
| H | CH(CH₃)CH₂SO₂CH₃ | CF₂Cl | Ph-4-OCF₃ |
| H | CH(CH₃)CH₂SO₂CH₃ | CF₂Cl | Ph-4-OCF₂Br |
| H | CH(CH₃)CH₂SO₂CH₃ | CF₂Cl | Ph-4-OCF₂CHFCF₃ |
| H | CH(CH₃)CH₂SO₂CH₃ | CF₂Cl | Ph-4-OCF₂CHFOCF₃ |
| H | CH(CH₃)CH₂SO₂CH₃ | CF₂Cl | Ph-4-OCF₂CHFOCF₂CF₂CF₃ |
| H | CH(CH₃)CH₂SO₂CH₃ | CF₂Cl | Ph-4-O(L-45g) |
| H | CH(CH₃)CH₂SO₂CH₃ | CF₂Cl | Ph-3,4-Cl₂ |
| H | CH(CH₃)CH₂SO₂CH₃ | CF₂Cl | Ph(-3-OCF₂O-4-) |
| H | CH(CH₃)CH₂SO₂CH₃ | CF₂Cl | Ph(-3-OCF₂CF₂O-4-) |
| H | CH(CH₃)CH₂SO₂CH₃ | CF₂Cl | (L-45c)CF₃ |
| H | CH(CH₃)CH₂SO₂CH₃ | CF₂Cl | L-45f |
| H | CH(CH₃)CH₂SO₂CH₃ | CF₂Cl | (L-46c)CF₃ |
| H | CH(CH₃)CH₂SO₂CH₃ | CF₂Cl | L-46d |
| H | CH(CH₃)CH₂SO₂CH₃ | CFCl₂ | Ph-4-CF₃ |
| H | CH(CH₃)CH₂SO₂CH₃ | CF₂Br | Ph-4-Cl |
| H | CH(CH₃)CH₂SO₂CH₃ | CF₂Br | Ph-4-Br |
| H | CH(CH₃)CH₂SO₂CH₃ | CF₂Br | Ph-4-CF₃ |
| H | CH(CH₃)CH₂SO₂CH₃ | CF₂Br | Ph-4-OCF₃ |
| H | CH(CH₃)CH₂SO₂CH₃ | CF₂Br | Ph-4-OCF₂Br |
| H | CH(CH₃)CH₂SO₂CH₃ | CF₂Br | Ph-4-OCF₂CHFCF₃ |
| H | CH(CH₃)CH₂SO₂CH₃ | CF₂Br | Ph-4-OCF₂CHFOCF₃ |
| H | CH(CH₃)CH₂SO₂CH₃ | CF₂Br | Ph-4-OCF₂CHFOCF₂CF₂CF₃ |
| H | CH(CH₃)CH₂SO₂CH₃ | CF₂Br | Ph-4-O(L-45g) |
| H | CH(CH₃)CH₂SO₂CH₃ | CF₂Br | Ph-3,4-Cl₂ |
| H | CH(CH₃)CH₂SO₂CH₃ | CF₂Br | Ph(-3-OCF₂O-4-) |
| H | CH(CH₃)CH₂SO₂CH₃ | CF₂Br | Ph(-3-OCF₂CF₂O-4-) |
| H | CH(CH₃)CH₂SO₂CH₃ | CF₂Br | (L-45c)CF₃ |
| H | CH(CH₃)CH₂SO₂CH₃ | CF₂Br | L-45f |
| H | CH(CH₃)CH₂SO₂CH₃ | CF₂Br | (L-46c)CF₃ |
| H | CH(CH₃)CH₂SO₂CH₃ | CF₂Br | L-46d |
| H | CH(CH₃)CH₂SO₂CH₃ | CFClBr | Ph-4-OCF₃ |
| H | CH(CH₃)CH₂SO₂CH₃ | CFBr₂ | (L-45c)CF₃ |
| H | CH(CH₃)CH₂SO₂CH₃ | CF₂CHF₂ | Ph-4-Br |
| H | CH(CH₃)CH₂SO₂CH₃ | CF₂CF₃ | Ph-4-Cl |
| H | CH(CH₃)CH₂SO₂CH₃ | CF₂CF₃ | Ph-4-Br |
| H | CH(CH₃)CH₂SO₂CH₃ | CF₂CF₃ | Ph-4-CF₃ |
| H | CH(CH₃)CH₂SO₂CH₃ | CF₂CF₃ | Ph-4-OCF₃ |
| H | CH(CH₃)CH₂SO₂CH₃ | CF₂CF₃ | Ph-4-OCF₂Br |
| H | CH(CH₃)CH₂SO₂CH₃ | CF₂CF₃ | Ph-4-OCF₂CHFCF₃ |
| H | CH(CH₃)CH₂SO₂CH₃ | CF₂CF₃ | Ph-4-OCF₂CHFOCF₃ |
| H | CH(CH₃)CH₂SO₂CH₃ | CF₂CF₃ | Ph-4-OCF₂CHFOCF₂CF₂CF₃ |
| H | CH(CH₃)CH₂SO₂CH₃ | CF₂CF₃ | Ph-4-O(L-45g) |
| H | CH(CH₃)CH₂SO₂CH₃ | CF₂CF₃ | Ph-3,4-Cl₂ |
| H | CH(CH₃)CH₂SO₂CH₃ | CF₂CF₃ | Ph(-3-OCF₂O-4-) |
| H | CH(CH₃)CH₂SO₂CH₃ | CF₂CF₃ | Ph(-3-OCF₂CF₂O-4-) |
| H | CH(CH₃)CH₂SO₂CH₃ | CF₂CF₃ | (L-45c)CF₃ |
| H | CH(CH₃)CH₂SO₂CH₃ | CF₂CF₃ | L-45f |
| H | CH(CH₃)CH₂SO₂CH₃ | CF₂CF₃ | (L-46c)CF₃ |
| H | CH(CH₃)CH₂SO₂CH₃ | CF₂CF₃ | L-46d |
| H | CH(CH₃)CH₂SO₂CH₃ | CF₂CF₂Cl | Ph-4-CF₃ |
| H | CH(CH₃)CH₂SO₂CH₃ | CFClCF₃ | Ph-4-OCF₃ |
| H | CH(CH₃)CH₂SO₂CH₃ | CFClCF₂Cl | (L-45c)CF₃ |
| H | CH(CH₃)CH₂SO₂CH₃ | CF₂CF₂Br | Ph-4-Br |
| H | CH(CH₃)CH₂SO₂CH₃ | CFBrCF₃ | Ph-4-CF₃ |
| H | CH(CH₃)CH₂SO₂CH₃ | CF₂CHFCF₃ | Ph-4-OCF₃ |
| H | CH(CH₃)CH₂SO₂CH₃ | CF₂CF₂CF₃ | Ph-4-Cl |
| H | CH(CH₃)CH₂SO₂CH₃ | CF₂CF₂CF₃ | Ph-4-Br |
| H | CH(CH₃)CH₂SO₂CH₃ | CF₂CF₂CF₃ | Ph-4-CF₃ |
| H | CH(CH₃)CH₂SO₂CH₃ | CF₂CF₂CF₃ | Ph-4-OCF₃ |
| H | CH(CH₃)CH₂SO₂CH₃ | CF₂CF₂CF₃ | Ph-4-OCF₂Br |
| H | CH(CH₃)CH₂SO₂CH₃ | CF₂CF₂CF₃ | Ph-4-OCF₂CHFCF₃ |
| H | CH(CH₃)CH₂SO₂CH₃ | CF₂CF₂CF₃ | Ph-4-OCF₂CHFOCF₃ |
| H | CH(CH₃)CH₂SO₂CH₃ | CF₂CF₂CF₃ | Ph-4-OCF₂CHFOCF₂CF₂CF₃ |
| H | CH(CH₃)CH₂SO₂CH₃ | CF₂CF₂CF₃ | Ph-4-O(L-45g) |
| H | CH(CH₃)CH₂SO₂CH₃ | CF₂CF₂CF₃ | Ph-3,4-Cl₂ |
| H | CH(CH₃)CH₂SO₂CH₃ | CF₂CF₂CF₃ | Ph(-3-OCF₂O-4-) |
| H | CH(CH₃)CH₂SO₂CH₃ | CF₂CF₂CF₃ | Ph(-3-OCF₂CF₂O-4-) |
| H | CH(CH₃)CH₂SO₂CH₃ | CF₂CF₂CF₃ | (L-45c)CF₃ |
| H | CH(CH₃)CH₂SO₂CH₃ | CF₂CF₂CF₃ | L-45f |
| H | CH(CH₃)CH₂SO₂CH₃ | CF₂CF₂CF₃ | (L-46c)CF₃ |
| H | CH(CH₃)CH₂SO₂CH₃ | CF₂CF₂CF₃ | L-46d |
| H | CH(CH₃)CH₂SO₂CH₃ | CF(CF₃)₂ | (L-45c)CF₃ |
| H | CH(CH₃)CH₂SO₂CH₃ | CF₂CF₂CF₂CF₃ | Ph-4-Br |
| H | CH(CH₃)CH₂SO₂CH₃ | T-1 | Ph-4-CF₃ |
| H | CH(CH₃)CH₂SO₂CH₃ | T-2 | Ph-4-OCF₃ |
| H | CH(CH₃)CH₂SO₂CH₃ | CH₂OCH₃ | (L-45c)CF₃ |
| H | CH(CH₃)CH₂SO₂CH₃ | CH₂OEt | Ph-4-Br |
| H | CH(CH₃)CH₂SO₂CH₃ | CH₂OCH₂CF₃ | Ph-4-CF₃ |
| H | CH(CH₃)CH₂SO₂CH₃ | CH₂OCH(CF₃)₂ | Ph-4-OCF₃ |
| H | CH(CH₃)CH₂SO₂CH₃ | CF₂OCH₃ | Ph-4-Cl |
| H | CH(CH₃)CH₂SO₂CH₃ | CF₂OCH₃ | Ph-4-Br |
| H | CH(CH₃)CH₂SO₂CH₃ | CF₂OCH₃ | Ph-4-CF₃ |
| H | CH(CH₃)CH₂SO₂CH₃ | CF₂OCH₃ | Ph-4-OCF₃ |
| H | CH(CH₃)CH₂SO₂CH₃ | CF₂OCH₃ | Ph-4-OCF₂Br |
| H | CH(CH₃)CH₂SO₂CH₃ | CF₂OCH₃ | Ph-4-OCF₂CHFCF₃ |
| H | CH(CH₃)CH₂SO₂CH₃ | CF₂OCH₃ | Ph-4-OCF₂CHFOCF₃ |
| H | CH(CH₃)CH₂SO₂CH₃ | CF₂OCH₃ | Ph-4-OCF₂CHFOCF₂CF₂CF₃ |
| H | CH(CH₃)CH₂SO₂CH₃ | CF₂OCH₃ | Ph-4-O(L-45g) |
| H | CH(CH₃)CH₂SO₂CH₃ | CF₂OCH₃ | Ph-3,4-Cl₂ |
| H | CH(CH₃)CH₂SO₂CH₃ | CF₂OCH₃ | Ph(-3-OCF₂O-4-) |
| H | CH(CH₃)CH₂SO₂CH₃ | CF₂OCH₃ | Ph(-3-OCF₂CF₂O-4-) |
| H | CH(CH₃)CH₂SO₂CH₃ | CF₂OCH₃ | (L-45c)CF₃ |
| H | CH(CH₃)CH₂SO₂CH₃ | CF₂OCH₃ | L-45f |
| H | CH(CH₃)CH₂SO₂CH₃ | CF₂OCH₃ | (L-46c)CF₃ |
| H | CH(CH₃)CH₂SO₂CH₃ | CF₂OCH₃ | L-46d |
| H | CH(CH₃)CH₂SO₂CH₃ | CH₂SCH₃ | (L-45c)CF₃ |
| H | CH(CH₃)CH₂SO₂CH₃ | CH₂SO₂CH₃ | Ph-4-Br |
| H | CH(CH₃)CH₂SO₂CH₃ | CH₂SCF₃ | Ph-4-CF₃ |
| H | CH(CH₃)CH₂SO₂CH₃ | CF₂SCH₃ | Ph-4-Cl |
| H | CH(CH₃)CH₂SO₂CH₃ | CF₂SCH₃ | Ph-4-Br |
| H | CH(CH₃)CH₂SO₂CH₃ | CF₂SCH₃ | Ph-4-CF₃ |
| H | CH(CH₃)CH₂SO₂CH₃ | CF₂SCH₃ | Ph-4-OCF₃ |
| H | CH(CH₃)CH₂SO₂CH₃ | CF₂SCH₃ | Ph-4-OCF₂Br |
| H | CH(CH₃)CH₂SO₂CH₃ | CF₂SCH₃ | Ph-4-OCF₂CHFCF₃ |
| H | CH(CH₃)CH₂SO₂CH₃ | CF₂SCH₃ | Ph-4-OCF₂CHFOCF₃ |
| H | CH(CH₃)CH₂SO₂CH₃ | CF₂SCH₃ | Ph-4-OCF₂CHFOCF₂CF₂CF₃ |
| H | CH(CH₃)CH₂SO₂CH₃ | CF₂SCH₃ | Ph-4-O(L-45g) |
| H | CH(CH₃)CH₂SO₂CH₃ | CF₂SCH₃ | Ph-3,4-Cl₂ |
| H | CH(CH₃)CH₂SO₂CH₃ | CF₂SCH₃ | Ph(-3-OCF₂O-4-) |
| H | CH(CH₃)CH₂SO₂CH₃ | CF₂SCH₃ | Ph(-3-OCF₂CF₂O-4-) |
| H | CH(CH₃)CH₂SO₂CH₃ | CF₂SCH₃ | (L-45c)CF₃ |
| H | CH(CH₃)CH₂SO₂CH₃ | CF₂SCH₃ | L-45f |
| H | CH(CH₃)CH₂SO₂CH₃ | CF₂SCH₃ | (L-46c)CF₃ |
| H | CH(CH₃)CH₂SO₂CH₃ | CF₂SCH₃ | L-46d |
| H | CH(CH₃)CH₂SO₂CH₃ | CF₂SEt | Ph-4-OCF₃ |
| H | CH(CH₃)CH₂SO₂CH₃ | CF₂SPr-n | (L-45c)CF₃ |
| H | CH(CH₃)CH₂SO₂CH₃ | CF₂SPr-i | Ph-4-Br |
| H | CH(CH₃)CH₂SO₂CH₃ | CF₂SCH₂Ph | Ph-4-CF₃ |
| H | CH(CH₃)CH₂SO₂CH₃ | CF₂SPh | Ph-4-OCF₃ |
| H | CH(CH₃)CH₂SO₂CH₃ | CF₂C(O)OEt | (L-45c)CF₃ |
| H | CH(CH₃)CH₂SO₂CH₃ | CF₂SO₂N(CH₃)₂ | Ph-4-Br |
| H | CH(CH₃)CH₂SO₂CH₃ | C(O)OEt | Ph-4-CF₃ |
| H | CH(CH₃)CH₂SO₂CH₃ | C(O)SEt | Ph-4-OCF₃ |
| H | CH(CH₃)CH₂SEt | CF₃ | CH═CH(Ph-4-Cl) |
| H | CH(CH₃)CH₂SEt | CF₃ | Ph-4-Cl |
| H | CH(CH₃)CH₂SEt | CF₃ | Ph-4-Br |
| H | CH(CH₃)CH₂SEt | CF₃ | Ph-4-CF₃ |
| H | CH(CH₃)CH₂SEt | CF₃ | Ph-4-OCF₃ |
| H | CH(CH₃)CH₂SEt(R) | CF₃ | Ph-4-OCF₃ |
| H | CH(CH₃)CH₂SEt(S) | CF₃ | Ph-4-OCF₃ |
| H | CH(CH₃)CH₂SEt | CF₃ | Ph-4-OCF₂Br |
| H | CH(CH₃)CH₂SEt | CF₃ | Ph-4-OCF₂CHFCF₃ |
| H | CH(CH₃)CH₂SEt | CF₃ | Ph-4-OCF₂CHFOCF₃ |
| H | CH(CH₃)CH₂SEt | CF₃ | Ph-4-OCF₂CHFOCF₂CF₂CF₃ |
| H | CH(CH₃)CH₂SEt | CF₃ | Ph-4-O(L-45g) |
| H | CH(CH₃)CH₂SEt | CF₃ | Ph-3,4-Cl₂ |
| H | CH(CH₃)CH₂SEt | CF₃ | Ph(-3-OCF₂O-4-) |
| H | CH(CH₃)CH₂SEt | CF₃ | Ph(-3-OCF₂CF₂O-4-) |
| H | CH(CH₃)CH₂SEt | CF₃ | (L-45c)CF₃ |
| H | CH(CH₃)CH₂SEt | CF₃ | L-45f |
| H | CH(CH₃)CH₂SEt | CF₃ | (L-46c)CF₃ |
| H | CH(CH₃)CH₂SEt | CF₃ | L-46d |
| H | CH(CH₃)CH₂S(O)Et | CF₃ | CH═CH(Ph-4-Cl) |
| H | CH(CH₃)CH₂S(O)Et | CF₃ | Ph-4-Cl |
| H | CH(CH₃)CH₂S(O)Et | CF₃ | Ph-4-Br |
| H | CH(CH₃)CH₂S(O)Et | CF₃ | Ph-4-CF₃ |
| H | CH(CH₃)CH₂S(O)Et | CF₃ | Ph-4-OCF₃ |
| H | CH(CH₃)CH₂S(O)Et | CF₃ | Ph-4-OCF₂Br |
| H | CH(CH₃)CH₂S(O)Et | CF₃ | Ph-4-OCF₂CHFCF₃ |
| H | CH(CH₃)CH₂S(O)Et | CF₃ | Ph-4-OCF₂CHFOCF₃ |
| H | CH(CH₃)CH₂S(O)Et | CF₃ | Ph-4-OCF₂CHFOCF₂CF₂CF₃ |
| H | CH(CH₃)CH₂S(O)Et | CF₃ | Ph-4-O(L-45g) |
| H | CH(CH₃)CH₂S(O)Et | CF₃ | Ph-3,4-Cl₂ |
| H | CH(CH₃)CH₂S(O)Et | CF₃ | Ph(-3-OCF₂O-4-) |
| H | CH(CH₃)CH₂S(O)Et | CF₃ | Ph(-3-OCF₂CF₂O-4-) |
| H | CH(CH₃)CH₂S(O)Et | CF₃ | (L-45c)CF₃ |
| H | CH(CH₃)CH₂S(O)Et | CF₃ | L-45f |
| H | CH(CH₃)CH₂S(O)Et | CF₃ | (L-46c)CF₃ |
| H | CH(CH₃)CH₂S(O)Et | CF₃ | L-46d |
| H | CH(CH₃)CH₂SO₂Et | CF₃ | CH═CH(Ph-4-Cl) |
| H | CH(CH₃)CH₂SO₂Et | CF₃ | Ph-4-Cl |
| H | CH(CH₃)CH₂SO₂Et | CF₃ | Ph-4-Br |
| H | CH(CH₃)CH₂SO₂Et | CF₃ | Ph-4-CF₃ |
| H | CH(CH₃)CH₂SO₂Et | CF₃ | Ph-4-OCF₃ |
| H | CH(CH₃)CH₂SO₂Et | CF₃ | Ph-4-OCF₂Br |
| H | CH(CH₃)CH₂SO₂Et | CF₃ | Ph-4-OCF₂CHFCF₃ |
| H | CH(CH₃)CH₂SO₂Et | CF₃ | Ph-4-OCF₂CHFOCF₃ |
| H | CH(CH₃)CH₂SO₂Et | CF₃ | Ph-4-OCF₂CHFOCF₂CF₂CF₃ |
| H | CH(CH₃)CH₂SO₂Et | CF₃ | Ph-4-O(L-45g) |
| H | CH(CH₃)CH₂SO₂Et | CF₃ | Ph-3,4-Cl₂ |
| H | CH(CH₃)CH₂SO₂Et | CF₃ | Ph(-3-OCF₂O-4-) |
| H | CH(CH₃)CH₂SO₂Et | CF₃ | Ph(-3-OCF₂CF₂O-4-) |
| H | CH(CH₃)CH₂SO₂Et | CF₃ | (L-45c)CF₃ |
| H | CH(CH₃)CH₂SO₂Et | CF₃ | L-45f |
| H | CH(CH₃)CH₂SO₂Et | CF₃ | (L-46c)CF₃ |
| H | CH(CH₃)CH₂SO₂Et | CF₃ | L-46d |
| H | CH(CH₃)CH₂SPr-n | CF₃ | Ph-4-Br |
| H | CH(CH₃)CH₂SPr-n | CF₃ | Ph-4-CF₃ |
| H | CH(CH₃)CH₂SPr-n | CF₃ | Ph-4-OCF₃ |
| H | CH(CH₃)CH₂SPr-n | CF₃ | (L-45c)CF₃ |
| H | CH(CH₃)CH₂SPr-i | CF₃ | (L-45c)CF₃ |
| H | CH(CH₃)CH₂SO₂Pr-i | CF₃ | Ph-4-Br |
| H | CH(CH₃)CH₂SBu-n | CF₃ | Ph-4-CF₃ |
| H | CH(CH₃)CH₂SBu-i | CF₃ | Ph-4-Br |
| H | CH(CH₃)CH₂SBu-i | CF₃ | Ph-4-CF₃ |
| H | CH(CH₃)CH₂SBu-i | CF₃ | Ph-4-OCF₃ |
| H | CH(CH₃)CH₂SBu-i | CF₃ | (L-45c)CF₃ |
| H | CH(CH₃)CH₂SBu-t | CF₃ | Ph-4-Br |
| H | CH(CH₃)CH₂SBu-t | CF₃ | Ph-4-CF₃ |
| H | CH(CH₃)CH₂SBu-t | CF₃ | Ph-4-OCF₃ |
| H | CH(CH₃)CH₂SBu-t | CF₃ | (L-45c)CF₃ |
| H | CH(CH₃)CH₂SO₂Bu-t | CF₃ | Ph-4-OCF₃ |
| H | CH(CH₃)CH₂SHex-n | CF₃ | (L-45c)CF₃ |
| H | CH(CH₃)CH₂SHex-c | CF₃ | Ph-4-Br |
| H | CH(CH₃)CH₂SCH₂CF₃ | CF₃ | Ph-4-CF₃ |
| H | CH(CH₃)CH₂S(O)CH₂CF₃ | CF₃ | Ph-4-OCF₃ |
| H | CH(CH₃)CH₂SO₂CH₂CF₃ | CF₃ | (L-45c)CF₃ |
| H | CH(CH₃)CH₂SCH₂CH₂OH | CF₃ | Ph-4-Br |
| H | CH(CH₃)CH₂SCH₂CH₂OCH₃ | CF₃ | Ph-4-CF₃ |
| H | CH(CH₃)CH₂S(O)CH₂CH₂OCH₃ | CF₃ | Ph-4-OCF₃ |
| H | CH(CH₃)CH₂SO₂CH₂CH₂OCH₃ | CF₃ | (L-45c)CF₃ |
| H | CH(CH₃)CH₂SCH₂CH₂OEt | CF₃ | Ph-4-Br |
| H | CH(CH₃)CH₂S(O)CH₂CH₂OEt | CF₃ | Ph-4-CF₃ |
| H | CH(CH₃)CH₂SO₂CH₂CH₂OEt | CF₃ | Ph-4-OCF₃ |
| H | CH(CH₃)CH₂SCH₂CH₂OC(O)CF₃ | CF₃ | (L-45c)CF₃ |
| H | CH(CH₃)CH₂SCH₂CH₂SCH₃ | CF₃ | Ph-4-Br |
| H | CH(CH₃)CH₂SCH₂Si(CH₃)₃ | CF₃ | Ph-4-Br |
| H | CH(CH₃)CH₂SCH₂Si(CH₃)₃ | CF₃ | Ph-4-CF₃ |
| H | CH(CH₃)CH₂SCH₂Si(CH₃)₃ | CF₃ | Ph-4-OCF₃ |
| H | CH(CH₃)CH₂SCH₂Si(CH₃)₃ | CF₃ | (L-45c)CF₃ |
| H | CH(CH₃)CH₂SCH₂C(O)CH₃ | CF₃ | Ph-4-CF₃ |
| H | CH(CH₃)CH₂SCH₂CH₂C(O)OCH₃ | CF₃ | Ph-4-OCF₃ |
| H | CH(CH₃)CH₂S(O)CH₂CH₂C(O)OCH₃ | CF₃ | (L-45c)CF₃ |
| H | CH(CH₃)CH₂SO₂CH₂CH₂C(O)OCH₃ | CF₃ | Ph-4-Br |
| H | CH(CH₃)CH₂SCH₂C(O)N(Et)₂ | CF₃ | Ph-4-CF₃ |
| H | CH(CH₃)CH₂S(O)CH₂C(O)N(Et)₂ | CF₃ | Ph-4-OCF₃ |
| H | CH(CH₃)CH₂SO₂CH₂C(O)N(Et)₂ | CF₃ | (L-45c)CF₃ |
| H | CH(CH₃)CH₂SCH₂(Ph-2,4-Cl₂) | CF₃ | Ph-4-Br |
| H | CH(CH₃)CH₂SCH₂CH═CH₂ | CF₃ | Ph-4-CF₃ |
| H | CH(CH₃)CH₂SCH₂C≡CH | CF₃ | Ph-4-OCF₃ |
| H | CH(CH₃)CH₂SC(O)CH₃ | CF₃ | (L-45c)CF₃ |
| H | CH(CH₃)CH₂SSCH₃ | CF₃ | CH═CH(Ph-4-Cl) |
| H | CH(CH₃)CH₂SSCH₃ | CF₃ | Ph-4-Cl |
| H | CH(CH₃)CH₂SSCH₃ | CF₃ | Ph-4-Br |
| H | CH(CH₃)CH₂SSCH₃ | CF₃ | Ph-4-CF₃ |
| H | CH(CH₃)CH₂SSCH₃ | CF₃ | Ph-4-OCF₃ |
| H | CH(CH₃)CH₂SSCH₃ | CF₃ | Ph-4-OCF₂Br |
| H | CH(CH₃)CH₂SSCH₃ | CF₃ | Ph-4-OCF₂CHFCF₃ |
| H | CH(CH₃)CH₂SSCH₃ | CF₃ | Ph-4-OCF₂CHFOCF₃ |
| H | CH(CH₃)CH₂SSCH₃ | CF₃ | Ph-4-OCF₂CHFOCF₂CF₂CF₃ |
| H | CH(CH₃)CH₂SSCH₃ | CF₃ | Ph-4-O(L-45g) |
| H | CH(CH₃)CH₂SSCH₃ | CF₃ | Ph-3,4-Cl₂ |
| H | CH(CH₃)CH₂SSCH₃ | CF₃ | Ph(-3-OCF₂O-4-) |
| H | CH(CH₃)CH₂SSCH₃ | CF₃ | Ph(-3-OCF₂CF₂O-4-) |
| H | CH(CH₃)CH₂SSCH₃ | CF₃ | (L-45c)CF₃ |
| H | CH(CH₃)CH₂SSCH₃ | CF₃ | L-45f |
| H | CH(CH₃)CH₂SSCH₃ | CF₃ | (L-46c)CF₃ |
| H | CH(CH₃)CH₂SSCH₃ | CF₃ | L-46d |
| H | CH(CH₃)CH₂SS(Ph-2-NO₂) | CF₃ | Ph-4-Br |
| H | CH(CH₃)CH₂SO₂NH₂ | CF₃ | Ph-4-CF₃ |
| H | CH(CH₃)CH₂SO₂NHCH₃ | CF₃ | Ph-4-Br |
| H | CH(CH₃)CH₂SO₂NHCH₃ | CF₃ | Ph-4-CF₃ |
| H | CH(CH₃)CH₂SO₂NHCH₃ | CF₃ | Ph-4-OCF₃ |
| H | CH(CH₃)CH₂SO₂NHCH₃ | CF₃ | (L-45c)CF₃ |
| H | CH(CH₃)CH₂SO₂NHEt | CF₃ | CF₂O(Ph-4-Cl) |
| H | CH(CH₃)CH₂SO₂NHEt | CF₃ | CH═CH(Ph-4-Cl) |
| H | CH(CH₃)CH₂SO₂NHEt | CF₃ | CH═CH(Ph-4-Br) |
| H | CH(CH₃)CH₂SO₂NHEt | CF₃ | CH═CH(Ph-4-CF₃) |
| H | CH(CH₃)CH₂SO₂NHEt | CF₃ | CH═CH(Ph-4-OCF₃) |
| H | CH(CH₃)CH₂SO₂NHEt | CF₃ | CH═CH(Ph-3,4-Cl₂) |
| H | CH(CH₃)CH₂SO₂NHEt | CF₃ | Ph-4-F |
| H | CH(CH₃)CH₂SO₂NHEt | CF₃ | Ph-4-Cl |
| H | CH(CH₃)CH₂SO₂NHEt | CF₃ | Ph-4-Br |
| H | CH(CH₃)CH₂SO₂NHEt | CF₃ | Ph-4-CF₃ |
| H | CH(CH₃)CH₂SO₂NHEt | CF₃ | Ph-4-OCF₃ |
| H | CH(CH₃)CH₂SO₂NHEt(S) | CF₃ | Ph-4-OCF₃ |
| H | CH(CH₃)CH₂SO₂NHEt | CF₃ | Ph-4-OCF₂Br |
| H | CH(CH₃)CH₂SO₂NHEt | CF₃ | Ph-4-OCF₂CHF₂ |
| H | CH(CH₃)CH₂SO₂NHEt | CF₃ | Ph-4-OCF₂CHFCF₃ |
| H | CH(CH₃)CH₂SO₂NHEt | CF₃ | Ph-4-OCF₂CHFOCF₃ |
| H | CH(CH₃)CH₂SO₂NHEt | CF₃ | Ph-4-OCF₂CHFOCF₂CF₂CF₃ |
| H | CH(CH₃)CH₂SO₂NHEt | CF₃ | Ph-4-O(L-45g) |
| H | CH(CH₃)CH₂SO₂NHEt | CF₃ | Ph-3,4-F₂ |
| H | CH(CH₃)CH₂SO₂NHEt | CF₃ | Ph-3-F-4-Cl |
| H | CH(CH₃)CH₂SO₂NHEt | CF₃ | Ph-3,4-Cl₂ |
| H | CH(CH₃)CH₂SO₂NHEt | CF₃ | Ph(-3-OCF₂O-4-) |
| H | CH(CH₃)CH₂SO₂NHEt | CF₃ | Ph(-3-OCF₂CF₂O-4-) |
| H | CH(CH₃)CH₂SO₂NHEt | CF₃ | (L-45c)CF₃ |
| H | CH(CH₃)CH₂SO₂NHEt | CF₃ | L-45f |
| H | CH(CH₃)CH₂SO₂NHEt | CF₃ | (L-46c)CF₃ |
| H | CH(CH₃)CH₂SO₂NHEt | CF₃ | L-46d |
| H | CH(CH₃)CH₂SO₂NHPr-n | CF₃ | Ph-4-OCF₃ |
| H | CH(CH₃)CH₂SO₂NHPr-i | CF₃ | (L-45c)CF₃ |
| H | CH(CH₃)CH₂SO₂NHBu-n | CF₃ | Ph-4-Br |
| H | CH(CH₃)CH₂SO₂NHBu-t | CF₃ | Ph-4-CF₃ |
| H | CH(CH₃)CH₂SO₂NHCH₂CH₂Cl | CF₃ | Ph-4-OCF₃ |
| H | CH(CH₃)CH₂SO₂NHCH₂CH₂OH | CF₃ | (L-45c)CF₃ |
| H | CH(CH₃)CH₂SO₂NHCH₂CH₂OCH₃ | CF₃ | Ph-4-Br |
| H | CH(CH₃)CH₂SO₂NHCH₂CH₂SCH₃ | CF₃ | Ph-4-CF₃ |
| H | CH(CH₃)CH₂SO₂NHCH₂CH₂S(O)CH₃ | CF₃ | Ph-4-OCF₃ |
| H | CH(CH₃)CH₂SO₂NHCH₂CH₂SO₂CH₃ | CF₃ | (L-45c)CF₃ |
| H | CH(CH₃)CH₂SO₂NHCH(CH₃)CH₂SCH₃ | CF₃ | Ph-4-Br |
| H | CH(CH₃)CH₂SO₂NHCH(CH₃)CH₂SCH₃(S, S) | CF₃ | Ph-4-CF₃ |
| H | CH(CH₃)CH₂SO₂NHCH(CH₃)CH₂S(O)CH₃ | CF₃ | Ph-4-OCF₃ |
| H | CH(CH₃)CH₂SO₂NHCH(CH₃)CH₂S(O)CH₃(S, S) | CF₃ | (L-45c)CF₃ |
| H | CH(CH₃)CH₂SO₂NHCH(CH₃)CH₂SO₂CH₃ | CF₃ | Ph-4-Br |
| H | CH(CH₃)CH₂SO₂NHCH(CH₃)CH₂SO₂CH₃(S, S) | CF₃ | Ph-4-CF₃ |
| H | CH(CH₃)CH₂SO₂NHC(CH₃)₂CH₂SCH₃ | CF₃ | Ph-4-OCF₃ |
| H | CH(CH₃)CH₂SO₂NHCH(CH₃)C(O)NH₂ | CF₃ | (L-45c)CF₃ |
| H | CH(CH₃)CH₂SO₂NHCH(CH₃)C(O)NHCH₃ | CF₃ | Ph-4-Br |
| H | CH(CH₃)CH₂SO₂NHCH(CH₃)C(O)NHEt | CF₃ | Ph-4-CF₃ |
| H | CH(CH₃)CH₂SO₂NHCH₂CH═CH₂ | CF₃ | Ph-4-OCF₃ |
| H | CH(CH₃)CH₂SO₂NHCH₂C≡CH | CF₃ | (L-45c)CF₃ |
| H | CH(CH₃)CH₂SO₂NHCH₂Ph | CF₃ | Ph-4-Br |
| H | CH(CH₃)CH₂SO₂NHCH₂Ph(S) | CF₃ | Ph-4-CF₃ |
| H | CH(CH₃)CH₂SO₂NHCH₂(Ph-2-F) | CF₃ | Ph-4-OCF₃ |
| H | CH(CH₃)CH₂SO₂NHCH₂(Ph-2-Cl) | CF₃ | (L-45c)CF₃ |
| H | CH(CH₃)CH₂SO₂NHCH₂(Ph-2-Br) | CF₃ | Ph-4-Br |
| H | CH(CH₃)CH₂SO₂NHCH₂(Ph-2-OCH₃) | CF₃ | Ph-4-CF₃ |
| H | CH(CH₃)CH₂SO₂NHCH₂(Ph-3-OCH₃) | CF₃ | Ph-4-OCF₃ |
| H | CH(CH₃)CH₂SO₂NHCH₂(Ph-4-OCH₃) | CF₃ | (L-45c)CF₃ |
| H | CH(CH₃)CH₂SO₂NHCH₂(Ph-4-SCF₃) | CF₃ | Ph-4-Br |
| H | CH(CH₃)CH₂SO₂NHCH₂(Ph-2-NO₂) | CF₃ | Ph-4-CF₃ |
| H | CH(CH₃)CH₂SO₂NHCH₂(Ph-3-NO₂) | CF₃ | Ph-4-OCF₃ |
| H | CH(CH₃)CH₂SO₂NHCH₂(Ph-4-NO₂) | CF₃ | (L-45c)CF₃ |
| H | CH(CH₃)CH₂SO₂NHCH₂(Ph-3-CN) | CF₃ | Ph-4-Br |
| H | CH(CH₃)CH₂SO₂NHCH₂(Ph-4-CN) | CF₃ | Ph-4-CF₃ |
| H | CH(CH₃)CH₂SO₂NHCH₂(L-45a) | CF₃ | Ph-4-OCF₃ |
| H | CH(CH₃)CH₂SO₂NHCH₂(L-46a) | CF₃ | (L-45c)CF₃ |
| H | CH(CH₃)CH₂SO₂NHCH₂(L-47a) | CF₃ | Ph-4-Br |
| H | CH(CH₃)CH₂SO₂NHCH(CH₃)Ph | CF₃ | Ph-4-CF₃ |
| H | CH(CH₃)CH₂SO₂NHCH(CH₃)Ph(R) | CF₃ | Ph-4-OCF₃ |
| H | CH(CH₃)CH₂SO₂NHCH(CH₃)Ph(S) | CF₃ | (L-45c)CF₃ |
| H | CH(CH₃)CH₂SO₂NHCH₂CH₂Ph | CF₃ | Ph-4-Br |
| H | CH(CH₃)CH₂SO₂NH(Ph-2-OCH₃) | CF₃ | Ph-4-CF₃ |
| H | CH(CH₃)CH₂SO₂N(CH₃)₂ | CF₃ | Ph-4-Br |
| H | CH(CH₃)CH₂SO₂N(CH₃)₂ | CF₃ | Ph-4-CF₃ |
| H | CH(CH₃)CH₂SO₂N(CH₃)₂ | CF₃ | Ph-4-OCF₃ |
| H | CH(CH₃)CH₂SO₂N(CH₃)₂ | CF₃ | (L-45c)CF₃ |
| H | CH(CH₃)CH₂SO₂N(CH₃)Et | CF₃ | Ph-4-OCF₃ |
| H | CH(CH₃)CH₂SO₂N(Et)₂ | CF₃ | CF₂O(Ph-4-Cl) |
| H | CH(CH₃)CH₂SO₂N(Et)₂ | CF₃ | CH═CH(Ph-4-Cl) |
| H | CH(CH₃)CH₂SO₂N(Et)₂ | CF₃ | CH═CH(Ph-4-Br) |
| H | CH(CH₃)CH₂SO₂N(Et)₂ | CF₃ | CH═CH(Ph-4-CF₃) |
| H | CH(CH₃)CH₂SO₂N(Et)₂ | CF₃ | CH═CH(Ph-4-OCF₃) |
| H | CH(CH₃)CH₂SO₂N(Et)₂ | CF₃ | CH═CH(Ph-3,4-Cl₂) |
| H | CH(CH₃)CH₂SO₂N(Et)₂ | CF₃ | Ph-4-F |
| H | CH(CH₃)CH₂SO₂N(Et)₂ | CF₃ | Ph-4-Cl |
| H | CH(CH₃)CH₂SO₂N(Et)₂ | CF₃ | Ph-4-Br |
| H | CH(CH₃)CH₂SO₂N(Et)₂ | CF₃ | Ph-4-CF₃ |
| H | CH(CH₃)CH₂SO₂N(Et)₂ | CF₃ | Ph-4-OCF₃ |
| H | CH(CH₃)CH₂SO₂N(Et)₂(S) | CF₃ | Ph-4-OCF₃ |
| H | CH(CH₃)CH₂SO₂N(Et)₂ | CF₃ | Ph-4-OCF₂Br |
| H | CH(CH₃)CH₂SO₂N(Et)₂ | CF₃ | Ph-4-OCF₂CHF₂ |
| H | CH(CH₃)CH₂SO₂N(Et)₂ | CF₃ | Ph-4-OCF₂CHFCF₃ |
| H | CH(CH₃)CH₂SO₂N(Et)₂ | CF₃ | Ph-4-OCF₂CHFOCF₃ |
| H | CH(CH₃)CH₂SO₂N(Et)₂ | CF₃ | Ph-4-OCF₂CHFOCF₂CF₂CF₃ |
| H | CH(CH₃)CH₂SO₂N(Et)₂ | CF₃ | Ph-4-O(L-45g) |
| H | CH(CH₃)CH₂SO₂N(Et)₂ | CF₃ | Ph-3,4-F₂ |
| H | CH(CH₃)CH₂SO₂N(Et)₂ | CF₃ | Ph-3-F-4-Cl |
| H | CH(CH₃)CH₂SO₂N(Et)₂ | CF₃ | Ph-3,4-Cl₂ |
| H | CH(CH₃)CH₂SO₂N(Et)₂ | CF₃ | Ph(-3-OCF₂O-4-) |
| H | CH(CH₃)CH₂SO₂N(Et)₂ | CF₃ | Ph(-3-OCF₂CF₂O-4-) |
| H | CH(CH₃)CH₂SO₂N(Et)₂ | CF₃ | (L-45c)CF₃ |
| H | CH(CH₃)CH₂SO₂N(Et)₂ | CF₃ | L-45f |
| H | CH(CH₃)CH₂SO₂N(Et)₂ | CF₃ | (L-46c)CF₃ |
| H | CH(CH₃)CH₂SO₂N(Et)₂ | CF₃ | L-46d |
| H | CH(CH₃)CH₂SO₂(T-19) | CF₃ | (L-45c)CF₃ |
| H | CH(CH₃)CH₂SO₂(T-20) | CF₃ | Ph-4-Br |
| H | CH(CH₃)CH₂SO₂(T-21) | CF₃ | Ph-4-CF₃ |
| H | CH(CH₃)CH₂SO₂N(CH₃)CH₂Ph | CF₃ | Ph-4-OCF₃ |
| H | CH(CH₃)CH₂SO₂N(Et)CH₂Ph | CF₃ | (L-45c)CF₃ |
| H | CH(CH₃)CH₂SC(O)NHEt | CF₃ | Ph-4-Br |
| H | CH(CH₃)CH₂SC(O)N(CH₃)₂ | CF₃ | Ph-4-CF₃ |
| H | CH(CH₃)CH₂SC(O)N(Et)₂ | CF₃ | Ph-4-OCF₃ |
| H | CH(CH₃)CH₂SC(S)NHCH₃ | CF₃ | (L-45c)CF₃ |
| H | CH(CH₃)CH₂SC(S)NHEt | CF₃ | Ph-4-Br |
| H | CH(CH₃)CH₂SC(S)N(CH₃)₂ | CF₃ | Ph-4-CF₃ |
| H | CH(CH₃)CH₂SPh | CF₃ | Ph-4-Br |
| H | CH(CH₃)CH₂SPh | CF₃ | Ph-4-CF₃ |
| H | CH(CH₃)CH₂SPh | CF₃ | Ph-4-OCF₃ |
| H | CH(CH₃)CH₂SPh | CF₃ | (L-45c)CF₃ |
| H | CH(CH₃)CH₂S(L-21a) | CF₃ | Ph-4-OCF₃ |
| H | CH(CH₃)CH₂SO₂(L-21a) | CF₃ | (L-45c)CF₃ |
| H | CH(CH₃)CH₂S(L-45a) | CF₃ | Ph-4-Br |
| H | CH(CH₃)CH₂S(L-45a) | CF₃ | Ph-4-CF₃ |
| H | CH(CH₃)CH₂S(L-45a) | CF₃ | Ph-4-OCF₃ |
| H | CH(CH₃)CH₂S(L-45a) | CF₃ | (L-45c)CF₃ |
| H | CH(CH₃)CH₂S(O)(L-45a) | CF₃ | Ph-4-Br |
| H | CH(CH₃)CH₂S(O)(L-45a) | CF₃ | Ph-4-CF₃ |
| H | CH(CH₃)CH₂S(O)(L-45a) | CF₃ | Ph-4-OCF₃ |
| H | CH(CH₃)CH₂S(O)(L-45a) | CF₃ | (L-45c)CF₃ |
| H | CH(CH₃)CH₂SO₂(L-45a) | CF₃ | Ph-4-Br |
| H | CH(CH₃)CH₂SO₂(L-45a) | CF₃ | Ph-4-CF₃ |
| H | CH(CH₃)CH₂SO₂(L-45a) | CF₃ | Ph-4-OCF₃ |
| H | CH(CH₃)CH₂SO₂(L-45a) | CF₃ | (L-45c)CF₃ |
| H | CH(CH₃)CH₂S(L-48a) | CF₃ | Ph-4-Br |
| H | CH(CH₃)CH₂S(O)(L-48a) | CF₃ | Ph-4-CF₃ |
| H | CH(CH₃)CH₂SO₂(L-48a) | CF₃ | Ph-4-OCF₃ |
| H | CH(CH₃)CH₂S(L-48b) | CF₃ | (L-45c)CF₃ |
| H | CH(CH₃)CH₂S(O)(L-48b) | CF₃ | Ph-4-Br |
| H | CH(CH₃)CH₂SO₂(L-48b) | CF₃ | Ph-4-CF₃ |
| H | CH(Et)CH₂SCH₃ | CF₃ | (L-45c)CF₃ |
| H | CH(CH₂SCH₃)₂ | CF₃ | Ph-4-Br |
| H | CH(Ph)CH₂SCH₃ | CF₃ | Ph-4-CF₃ |
| H | CH(Ph)CH₂S(O)CH₃ | CF₃ | Ph-4-OCF₃ |
| H | CH(Ph)CH₂SO₂CH₃ | CF₃ | (L-45c)CF₃ |
| H | CH(CH₃)CH(CH₃)SCH₃ | CF₃ | Ph-4-Br |
| H | CH(CH₃)CH(CH₃)SO₂CH₃ | CF₃ | Ph-4-CF₃ |
| H | CH(CH₃)CH(CH₃)SEt | CF₃ | Ph-4-OCF₃ |
| H | CH(CH₃)CH(CH₃)SO₂Et | CF₃ | (L-45c)CF₃ |
| H | C(CH₃)₂CH₂SCH₃ | CH₃ | CF₂O(Ph-4-Cl) |
| H | C(CH₃)₂CH₂SCH₃ | CH₃ | CH═CH(Ph-4-Cl) |
| H | C(CH₃)₂CH₂SCH₃ | CH₃ | CH═CH(Ph-4-OCF₃) |
| H | C(CH₃)₂CH₂SCH₃ | CH₃ | CH═CH(Ph-3,4-Cl₂) |
| H | C(CH₃)₂CH₂SCH₃ | CH₃ | Ph-4-F |
| H | C(CH₃)₂CH₂SCH₃ | CH₃ | Ph-4-Cl |
| H | C(CH₃)₂CH₂SCH₃ | CH₃ | Ph-4-Br |
| H | C(CH₃)₂CH₂SCH₃ | CH₃ | Ph-4-I |
| H | C(CH₃)₂CH₂SCH₃ | CH₃ | Ph-4-CF₃ |
| H | C(CH₃)₂CH₂SCH₃ | CH₃ | Ph-4-OCHF₂ |
| H | C(CH₃)₂CH₂SCH₃ | CH₃ | Ph-4-OCF₃ |
| H | C(CH₃)₂CH₂SCH₃ | CH₃ | Ph-4-OCF₂Br |
| H | C(CH₃)₂CH₂SCH₃ | CH₃ | Ph-4-OCF₂CHF₂ |
| H | C(CH₃)₂CH₂SCH₃ | CH₃ | Ph-4-OCF₂CHFCl |
| H | C(CH₃)₂CH₂SCH₃ | CH₃ | Ph-4-OCF₂CHFCF₃ |
| H | C(CH₃)₂CH₂SCH₃ | CH₃ | Ph-4-OCF₂CHFOCF₃ |
| H | C(CH₃)₂CH₂SCH₃ | CH₃ | Ph-4-OCF₂CHFOCF₂CF₂CF₃ |
| H | C(CH₃)₂CH₂SCH₃ | CH₃ | Ph-4-O(L-45g) |
| H | C(CH₃)₂CH₂SCH₃ | CH₃ | Ph-3,4-Cl₂ |
| H | C(CH₃)₂CH₂SCH₃ | CH₃ | Ph(-3-OCF₂O-4-) |
| H | C(CH₃)₂CH₂SCH₃ | CH₃ | Ph(-3-OCF₂CF₂O-4-) |
| H | C(CH₃)₂CH₂SCH₃ | CH₃ | (L-45c)Cl |
| H | C(CH₃)₂CH₂SCH₃ | CH₃ | (L-45c)Br |
| H | C(CH₃)₂CH₂SCH₃ | CH₃ | (L-45c)CF₃ |
| H | C(CH₃)₂CH₂SCH₃ | CH₃ | L-45e |
| H | C(CH₃)₂CH₂SCH₃ | CH₃ | L-45f |
| H | C(CH₃)₂CH₂SCH₃ | CH₃ | (L-46c)Cl |
| H | C(CH₃)₂CH₂SCH₃ | CH₃ | (L-46c)Br |
| H | C(CH₃)₂CH₂SCH₃ | CH₃ | (L-46c)CF₃ |
| H | C(CH₃)₂CH₂SCH₃ | CH₃ | L-46d |
| H | C(CH₃)₂CH₂SCH₃ | Et | Ph-4-Cl |
| H | C(CH₃)₂CH₂SCH₃ | Et | Ph-4-Br |
| H | C(CH₃)₂CH₂SCH₃ | Et | Ph-4-CF₃ |
| H | C(CH₃)₂CH₂SCH₃ | Et | Ph-4-OCF₃ |
| H | C(CH₃)₂CH₂SCH₃ | Et | Ph-4-OCF₂Br |
| H | C(CH₃)₂CH₂SCH₃ | Et | Ph-4-OCF₂CHFCF₃ |
| H | C(CH₃)₂CH₂SCH₃ | Et | Ph-4-OCF₂CHFOCF₃ |
| H | C(CH₃)₂CH₂SCH₃ | Et | Ph-4-OCF₂CHFOCF₂CF₂CF₃ |
| H | C(CH₃)₂CH₂SCH₃ | Et | Ph-4-O(L-45g) |
| H | C(CH₃)₂CH₂SCH₃ | Et | Ph-3,4-Cl₂ |
| H | C(CH₃)₂CH₂SCH₃ | Et | Ph(-3-OCF₂O-4-) |
| H | C(CH₃)₂CH₂SCH₃ | Et | Ph(-3-OCF₂CF₂O-4-) |
| H | C(CH₃)₂CH₂SCH₃ | Et | (L-45c)CF₃ |
| H | C(CH₃)₂CH₂SCH₃ | n-Pr | Ph-4-Br |
| H | C(CH₃)₂CH₂SCH₃ | n-Pr | Ph-4-CF₃ |
| H | C(CH₃)₂CH₂SCH₃ | n-Pr | Ph-4-OCF₃ |
| H | C(CH₃)₂CH₂SCH₃ | n-Pr | (L-45c)CF₃ |
| H | C(CH₃)₂CH₂SCH₃ | i-Pr | Ph-4-Br |
| H | C(CH₃)₂CH₂SCH₃ | i-Pr | Ph-4-CF₃ |
| H | C(CH₃)₂CH₂SCH₃ | i-Pr | Ph-4-OCF₃ |
| H | C(CH₃)₂CH₂SCH₃ | i-Pr | (L-45c)CF₃ |
| H | C(CH₃)₂CH₂SCH₃ | c-Pr | Ph-4-Br |
| H | C(CH₃)₂CH₂SCH₃ | c-Pr | Ph-4-CF₃ |
| H | C(CH₃)₂CH₂SCH₃ | c-Pr | Ph-4-OCF₃ |
| H | C(CH₃)₂CH₂SCH₃ | c-Pr | (L-45c)CF₃ |
| H | C(CH₃)₂CH₂SCH₃ | n-Bu | Ph-4-Br |
| H | C(CH₃)₂CH₂SCH₃ | CH₂F | Ph-4-CF₃ |
| H | C(CH₃)₂CH₂SCH₃ | CH₂Cl | Ph-4-OCF₃ |
| H | C(CH₃)₂CH₂SCH₃ | CH₂Br | (L-45c)CF₃ |
| H | C(CH₃)₂CH₂SCH₃ | CHF₂ | Ph-4-Cl |
| H | C(CH₃)₂CH₂SCH₃ | CHF₂ | Ph-4-Br |
| H | C(CH₃)₂CH₂SCH₃ | CHF₂ | Ph-4-CF₃ |
| H | C(CH₃)₂CH₂SCH₃ | CHF₂ | Ph-4-OCF₃ |
| H | C(CH₃)₂CH₂SCH₃ | CHF₂ | Ph-4-OCF₂Br |
| H | C(CH₃)₂CH₂SCH₃ | CHF₂ | Ph-4-OCF₂CHFCF₃ |
| H | C(CH₃)₂CH₂SCH₃ | CHF₂ | Ph-4-OCF₂CHFOCF₃ |
| H | C(CH₃)₂CH₂SCH₃ | CHF₂ | Ph-4-OCF₂CHFOCF₂CF₂CF₃ |
| H | C(CH₃)₂CH₂SCH₃ | CHF₂ | Ph-4-O(L-45g) |
| H | C(CH₃)₂CH₂SCH₃ | CHF₂ | Ph-3,4-Cl₂ |
| H | C(CH₃)₂CH₂SCH₃ | CHF₂ | Ph(-3-OCF₂O-4-) |
| H | C(CH₃)₂CH₂SCH₃ | CHF₂ | Ph(-3-OCF₂CF₂O-4-) |
| H | C(CH₃)₂CH₂SCH₃ | CHF₂ | (L-45c)CF₃ |
| H | C(CH₃)₂CH₂SCH₃ | CHF₂ | L-45f |
| H | C(CH₃)₂CH₂SCH₃ | CHF₂ | (L-46c)CF₃ |
| H | C(CH₃)₂CH₂SCH₃ | CHF₂ | L-46d |
| H | C(CH₃)₂CH₂SCH₃ | CHFCl | Ph-4-Br |
| H | C(CH₃)₂CH₂SCH₃ | CHFBr | Ph-4-CF₃ |
| H | C(CH₃)₂CH₂SCH₃ | CF₃ | c-Pr |
| H | C(CH₃)₂CH₂SCH₃ | CF₃ | c-Bu |
| H | C(CH₃)₂CH₂SCH₃ | CF₃ | c-Pen |
| H | C(CH₃)₂CH₂SCH₃ | CF₃ | c-Hex |
| H | C(CH₃)₂CH₂SCH₃ | CF₃ | T-1 |
| H | C(CH₃)₂CH₂SCH₃ | CF₃ | T-2 |
| H | C(CH₃)₂CH₂SCH₃ | CF₃ | T-3 |
| H | C(CH₃)₂CH₂SCH₃ | CF₃ | T-4 |
| H | C(CH₃)₂CH₂SCH₃ | CF₃ | T-5 |
| H | C(CH₃)₂CH₂SCH₃ | CF₃ | CH₂OCH₃ |
| H | C(CH₃)₂CH₂SCH₃ | CF₃ | CH₂OEt |
| H | C(CH₃)₂CH₂SCH₃ | CF₃ | CH₂OPr-n |
| H | C(CH₃)₂CH₂SCH₃ | CF₃ | CH₂OPr-i |
| H | C(CH₃)₂CH₂SCH₃ | CF₃ | CH₂OBu-n |
| H | C(CH₃)₂CH₂SCH₃ | CF₃ | CH₂OC(O)(Ph-2-Cl) |
| H | C(CH₃)₂CH₂SCH₃ | CF₃ | CH₂OC(O)(Ph-3-Cl) |
| H | C(CH₃)₂CH₂SCH₃ | CF₃ | CH₂OC(O)(Ph-4-Cl) |
| H | C(CH₃)₂CH₂SCH₃ | CF₃ | CH₂OPh |
| H | C(CH₃)₂CH₂SCH₃ | CF₃ | CH₂O(Ph-2-F) |
| H | C(CH₃)₂CH₂SCH₃ | CF₃ | CH₂O(Ph-3-F) |
| H | C(CH₃)₂CH₂SCH₃ | CF₃ | CH₂O(Ph-4-F) |
| H | C(CH₃)₂CH₂SCH₃ | CF₃ | CH₂O(Ph-2-Cl) |
| H | C(CH₃)₂CH₂SCH₃ | CF₃ | CH₂O(Ph-3-Cl) |
| H | C(CH₃)₂CH₂SCH₃ | CF₃ | CH₂O(Ph-4-Cl) |
| H | C(CH₃)₂CH₂SCH₃ | CF₃ | CH₂O(Ph-2-Br) |
| H | C(CH₃)₂CH₂SCH₃ | CF₃ | CH₂O(Ph-3-Br) |
| H | C(CH₃)₂CH₂SCH₃ | CF₃ | CH₂O(Ph-4-Br) |
| H | C(CH₃)₂CH₂SCH₃ | CF₃ | CH₂O(Ph-2-CF₃) |
| H | C(CH₃)₂CH₂SCH₃ | CF₃ | CH₂O(Ph-3-CF₃) |
| H | C(CH₃)₂CH₂SCH₃ | CF₃ | CH₂O(Ph-4-CF₃) |
| H | C(CH₃)₂CH₂SCH₃ | CF₃ | CH₂O(Ph-2-OCF₃) |
| H | C(CH₃)₂CH₂SCH₃ | CF₃ | CH₂O(Ph-3-OCF₃) |
| H | C(CH₃)₂CH₂SCH₃ | CF₃ | CH₂O(Ph-4-OCF₃) |
| H | C(CH₃)₂CH₂SCH₃ | CF₃ | CF₂OPh |
| H | C(CH₃)₂CH₂SCH₃ | CF₃ | CF₂O(Ph-2-F) |
| H | C(CH₃)₂CH₂SCH₃ | CF₃ | CF₂O(Ph-3-F) |
| H | C(CH₃)₂CH₂SCH₃ | CF₃ | CF₂O(Ph-4-F) |
| H | C(CH₃)₂CH₂SCH₃ | CF₃ | CF₂O(Ph-2-Cl) |
| H | C(CH₃)₂CH₂SCH₃ | CF₃ | CF₂O(Ph-3-Cl) |
| H | C(CH₃)₂CH₂SCH₃ | CF₃ | CF₂O(Ph-4-Cl) |
| H | C(CH₃)₂CH₂SCH₃ | CF₃ | CF₂O(Ph-2-Br) |
| H | C(CH₃)₂CH₂SCH₃ | CF₃ | CF₂O(Ph-3-Br) |
| H | C(CH₃)₂CH₂SCH₃ | CF₃ | CF₂O(Ph-4-Br) |
| H | C(CH₃)₂CH₂SCH₃ | CF₃ | CF₂O(Ph-2-CF₃) |
| H | C(CH₃)₂CH₂SCH₃ | CF₃ | CF₂O(Ph-3-CF₃) |
| H | C(CH₃)₂CH₂SCH₃ | CF₃ | CF₂O(Ph-4-CF₃) |
| H | C(CH₃)₂CH₂SCH₃ | CF₃ | CF₂O(Ph-2-OCF₃) |
| H | C(CH₃)₂CH₂SCH₃ | CF₃ | CF₂O(Ph-3-OCF₃) |
| H | C(CH₃)₂CH₂SCH₃ | CF₃ | CF₂O(Ph-4-OCF₃) |
| H | C(CH₃)₂CH₂SCH₃ | CF₃ | CH₂S(Ph-2-Cl) |
| H | C(CH₃)₂CH₂SCH₃ | CF₃ | CH₂S(Ph-3-Cl) |
| H | C(CH₃)₂CH₂SCH₃ | CF₃ | CH₂S(Ph-4-Cl) |
| H | C(CH₃)₂CH₂SCH₃ | CF₃ | CH₂SO₂(Ph-2-Cl) |
| H | C(CH₃)₂CH₂SCH₃ | CF₃ | CH₂SO₂(Ph-3-Cl) |
| H | C(CH₃)₂CH₂SCH₃ | CF₃ | CH₂SO₂(Ph-4-Cl) |
| H | C(CH₃)₂CH₂SCH₃ | CF₃ | CH₂N(CH₃)₂ |
| H | C(CH₃)₂CH₂SCH₃ | CF₃ | CH₂NH(Ph-2-Cl) |
| H | C(CH₃)₂CH₂SCH₃ | CF₃ | CH₂NH(Ph-3-Cl) |
| H | C(CH₃)₂CH₂SCH₃ | CF₃ | CH₂NH(Ph-4-Cl) |
| H | C(CH₃)₂CH₂SCH₃ | CF₃ | CF₂C(O)OEt |
| H | C(CH₃)₂CH₂SCH₃ | CF₃ | CH₂(L-5a) |
| H | C(CH₃)₂CH₂SCH₃ | CF₃ | CH₂(L-14a) |
| H | C(CH₃)₂CH₂SCH₃ | CF₃ | CH₂(L-24a) |
| H | C(CH₃)₂CH₂SCH₃ | CF₃ | CH₂(L-36a) |
| H | C(CH₃)₂CH₂SCH₃ | CF₃ | CH₂CH₂Ph |
| H | C(CH₃)₂CH₂SCH₃ | CF₃ | CH₂CH₂(Ph-2-Cl) |
| H | C(CH₃)₂CH₂SCH₃ | CF₃ | CH₂CH₂(Ph-3-Cl) |
| H | C(CH₃)₂CH₂SCH₃ | CF₃ | CH₂CH₂(Ph-4-Cl) |
| H | C(CH₃)₂CH₂SCH₃ | CF₃ | T-22 |
| H | C(CH₃)₂CH₂SCH₃ | CF₃ | T-23 |
| H | C(CH₃)₂CH₂SCH₃ | CF₃ | T-24 |
| H | C(CH₃)₂CH₂SCH₃ | CF₃ | C(O)OEt |
| H | C(CH₃)₂CH₂SCH₃ | CF₃ | C(O)OBu-t |
| H | C(CH₃)₂CH₂SCH₃ | CF₃ | C(O)OCH₂CF₃ |
| H | C(CH₃)₂CH₂SCH₃ | CF₃ | CH═CH(Ph-2-F) |
| H | C(CH₃)₂CH₂SCH₃ | CF₃ | CH═CH(Ph-3-F) |
| H | C(CH₃)₂CH₂SCH₃ | CF₃ | CH═CH(Ph-4-F) |
| H | C(CH₃)₂CH₂SCH₃ | CF₃ | CH═CH(Ph-2-Cl) |
| H | C(CH₃)₂CH₂SCH₃ | CF₃ | CH═CH(Ph-3-Cl) |
| H | C(CH₃)₂CH₂SCH₃ | CF₃ | CH═CH(Ph-4-Cl) |
| H | C(CH₃)₂CH₂SCH₃ | CF₃ | CH═CH(Ph-2-Br) |
| H | C(CH₃)₂CH₂SCH₃ | CF₃ | CH═CH(Ph-3-Br) |
| H | C(CH₃)₂CH₂SCH₃ | CF₃ | CH═CH(Ph-4-Br) |
| H | C(CH₃)₂CH₂SCH₃ | CF₃ | CH═CH(Ph-2-CF₃) |
| H | C(CH₃)₂CH₂SCH₃ | CF₃ | CH═CH(Ph-3-CF₃) |
| H | C(CH₃)₂CH₂SCH₃ | CF₃ | CH═CH(Ph-4-CF₃) |
| H | C(CH₃)₂CH₂SCH₃ | CF₃ | CH═CH(Ph-2-OCF₃) |
| H | C(CH₃)₂CH₂SCH₃ | CF₃ | CH═CH(Ph-3-OCF₃) |
| H | C(CH₃)₂CH₂SCH₃ | CF₃ | CH═CH(Ph-4-OCF₃) |
| H | C(CH₃)₂CH₂SCH₃ | CF₃ | CH═CH(Ph-2-SCH₃) |
| H | C(CH₃)₂CH₂SCH₃ | CF₃ | CH═CH(Ph-3-SCH₃) |
| H | C(CH₃)₂CH₂SCH₃ | CF₃ | CH═CH(Ph-4-SCH₃) |
| H | C(CH₃)₂CH₂SCH₃ | CF₃ | CH═CH(Ph-2-SO₂CH₃) |
| H | C(CH₃)₂CH₂SCH₃ | CF₃ | CH═CH(Ph-3-SO₂CH₃) |
| H | C(CH₃)₂CH₂SCH₃ | CF₃ | CH═CH(Ph-4-SO₂CH₃) |
| H | C(CH₃)₂CH₂SCH₃ | CF₃ | CH═CH(Ph-3,4-F₂) |
| H | C(CH₃)₂CH₂SCH₃ | CF₃ | CH═CH(Ph-3-F-4-Cl) |
| H | C(CH₃)₂CH₂SCH₃ | CF₃ | CH═CH(Ph-3,4-Cl₂) |
| H | C(CH₃)₂CH₂SCH₃ | CF₃ | CH═CH(Ph-3,4-Br₂) |
| H | C(CH₃)₂CH₂SCH₃ | CF₃ | CH═CH(Ph-3-F-4-CF₃) |
| H | C(CH₃)₂CH₂SCH₃ | CF₃ | CH═CH(Ph-3-Cl-4-OCF₃) |
| H | C(CH₃)₂CH₂SCH₃ | CF₃ | CH═CH[Ph(-3-OCF₂O-4-)] |
| H | C(CH₃)₂CH₂SCH₃ | CF₃ | Ph |
| H | C(CH₃)₂CH₂SCH₃ | CF₃ | Ph-2-F |
| H | C(CH₃)₂CH₂SCH₃ | CF₃ | Ph-3-F |
| H | C(CH₃)₂CH₂SCH₃ | CF₃ | Ph-4-F |
| H | C(CH₃)₂CH₂SCH₃ | CF₃ | Ph-2-Cl |
| H | C(CH₃)₂CH₂SCH₃ | CF₃ | Ph-3-Cl |
| H | C(CH₃)₂CH₂SCH₃ | CF₃ | Ph-4-Cl |
| H | C(CH₃)₂CH₂SCH₃ | CF₃ | Ph-2-Br |
| H | C(CH₃)₂CH₂SCH₃ | CF₃ | Ph-3-Br |
| H | C(CH₃)₂CH₂SCH₃ | CF₃ | Ph-4-Br |
| H | C(CH₃)₂CH₂SCH₃ | CF₃ | Ph-4-I |
| H | C(CH₃)₂CH₂SCH₃ | CF₃ | Ph-4-Bu-t |
| H | C(CH₃)₂CH₂SCH₃ | CF₃ | Ph-2-CF₃ |
| H | C(CH₃)₂CH₂SCH₃ | CF₃ | Ph-3-CF₃ |
| H | C(CH₃)₂CH₂SCH₃ | CF₃ | Ph-4-CF₃ |
| H | C(CH₃)₂CH₂SCH₃ | CF₃ | Ph-4-OCH₃ |
| H | C(CH₃)₂CH₂SCH₃ | CF₃ | Ph-4-OCHF₂ |
| H | C(CH₃)₂CH₂SCH₃ | CF₃ | Ph-2-OCF₃ |
| H | C(CH₃)₂CH₂SCH₃ | CF₃ | Ph-3-OCF₃ |
| H | C(CH₃)₂CH₂SCH₃ | CF₃ | Ph-4-OCF₃ |
| H | C(CH₃)₂CH₂SCH₃ | CF₃ | Ph-4-OCF₂Br |
| H | C(CH₃)₂CH₂SCH₃ | CF₃ | Ph-4-OCH₂CF₃ |
| H | C(CH₃)₂CH₂SCH₃ | CF₃ | Ph-4-OCF₂CHF₂ |
| H | C(CH₃)₂CH₂SCH₃ | CF₃ | Ph-4-OCF₂CHFCl |
| H | C(CH₃)₂CH₂SCH₃ | CF₃ | Ph-4-OCF₂CHFBr |
| H | C(CH₃)₂CH₂SCH₃ | CF₃ | Ph-4-OCF₂CF₂Br |
| H | C(CH₃)₂CH₂SCH₃ | CF₃ | Ph-4-OCF₂CFCl₂ |
| H | C(CH₃)₂CH₂SCH₃ | CF₃ | Ph-4-OCF₂CCl₃ |
| H | C(CH₃)₂CH₂SCH₃ | CF₃ | Ph-4-OCH₂CF₂CHF₂ |
| H | C(CH₃)₂CH₂SCH₃ | CF₃ | Ph-3-OCF₂CHFCF₃ |
| H | C(CH₃)₂CH₂SCH₃ | CF₃ | Ph-4-OCF₂CHFCF₃ |
| H | C(CH₃)₂CH₂SCH₃ | CF₃ | Ph-4-OCH(CF₃)₂ |
| H | C(CH₃)₂CH₂SCH₃ | CF₃ | Ph-4-OCF₂CFBrCF₃ |
| H | C(CH₃)₂CH₂SCH₃ | CF₃ | Ph-3-OCF₂CHFOCF₃ |
| H | C(CH₃)₂CH₂SCH₃ | CF₃ | Ph-4-OCF₂CHFOCF₃ |
| H | C(CH₃)₂CH₂SCH₃ | CF₃ | Ph-4-OCF₂CHFOCF₂CF₂CF₃ |
| H | C(CH₃)₂CH₂SCH₃ | CF₃ | Ph-4-OSO₂CH₃ |
| H | C(CH₃)₂CH₂SCH₃ | CF₃ | Ph-4-OSO₂CF₃ |
| H | C(CH₃)₂CH₂SCH₃ | CF₃ | Ph-3-O(Ph-4-Cl) |
| H | C(CH₃)₂CH₂SCH₃ | CF₃ | Ph-4-O(Ph-4-Cl) |
| H | C(CH₃)₂CH₂SCH₃ | CF₃ | Ph-4-O(Ph-4-Br) |
| H | C(CH₃)₂CH₂SCH₃ | CF₃ | Ph-4-O(Ph-4-CF₃) |
| H | C(CH₃)₂CH₂SCH₃ | CF₃ | Ph-4-O(L-45c)Br |
| H | C(CH₃)₂CH₂SCH₃ | CF₃ | Ph-4-O(L-45c)CF₃ |
| H | C(CH₃)₂CH₂SCH₃ | CF₃ | Ph-3-O(L-45e) |
| H | C(CH₃)₂CH₂SCH₃ | CF₃ | Ph-4-O(L-45e) |
| H | C(CH₃)₂CH₂SCH₃ | CF₃ | Ph-4-O(L-48b)Br |
| H | C(CH₃)₂CH₂SCH₃ | CF₃ | Ph-4-SCH₃ |
| H | C(CH₃)₂CH₂SCH₃ | CF₃ | Ph-4-SO₂CH₃ |
| H | C(CH₃)₂CH₂SCH₃ | CF₃ | Ph-3-S(Ph-4-Cl) |
| H | C(CH₃)₂CH₂SCH₃ | CF₃ | Ph-4-S(Ph-4-Cl) |
| H | C(CH₃)₂CH₂SCH₃ | CF₃ | Ph-4-S(Ph-4-Br) |
| H | C(CH₃)₂CH₂SCH₃ | CF₃ | Ph-4-S(Ph-4-CF₃) |
| H | C(CH₃)₂CH₂SCH₃ | CF₃ | Ph-4-S(L-45c)Br |
| H | C(CH₃)₂CH₂SCH₃ | CF₃ | Ph-4-S(L-45c)CF₃ |
| H | C(CH₃)₂CH₂SCH₃ | CF₃ | Ph-4-S(L-45e) |
| H | C(CH₃)₂CH₂SCH₃ | CF₃ | Ph-4-S(L-48b)Br |
| H | C(CH₃)₂CH₂SCH₃ | CF₃ | Ph-4-NO₂ |
| H | C(CH₃)₂CH₂SCH₃ | CF₃ | Ph-4-CN |
| H | C(CH₃)₂CH₂SCH₃ | CF₃ | Ph-2,3-F₂ |
| H | C(CH₃)₂CH₂SCH₃ | CF₃ | Ph-2,4-F₂ |
| H | C(CH₃)₂CH₂SCH₃ | CF₃ | Ph-3,4-F₂ |
| H | C(CH₃)₂CH₂SCH₃ | CF₃ | Ph-2,5-F₂ |
| H | C(CH₃)₂CH₂SCH₃ | CF₃ | Ph-3,5-F₂ |
| H | C(CH₃)₂CH₂SCH₃ | CF₃ | Ph-2-Cl-4-F |
| H | C(CH₃)₂CH₂SCH₃ | CF₃ | Ph-2-F-3-Cl |
| H | C(CH₃)₂CH₂SCH₃ | CF₃ | Ph-3-Cl-4-F |
| H | C(CH₃)₂CH₂SCH₃ | CF₃ | Ph-2-F-4-Cl |
| H | C(CH₃)₂CH₂SCH₃ | CF₃ | Ph-3-F-4-Cl |
| H | C(CH₃)₂CH₂SCH₃ | CF₃ | Ph-2,3-Cl₂ |
| H | C(CH₃)₂CH₂SCH₃ | CF₃ | Ph-2,4-Cl₂ |
| H | C(CH₃)₂CH₂SCH₃ | CF₃ | Ph-2,5-Cl₂ |
| H | C(CH₃)₂CH₂SCH₃ | CF₃ | Ph-3,4-Cl₂ |
| H | C(CH₃)₂CH₂SCH₃ | CF₃ | Ph-3,5-Cl₂ |
| H | C(CH₃)₂CH₂SCH₃ | CF₃ | Ph-3-Br-4-F |
| H | C(CH₃)₂CH₂SCH₃ | CF₃ | Ph-2-F-4-Br |
| H | C(CH₃)₂CH₂SCH₃ | CF₃ | Ph-2-F-5-Br |
| H | C(CH₃)₂CH₂SCH₃ | CF₃ | Ph-3,4-Br₂ |
| H | C(CH₃)₂CH₂SCH₃ | CF₃ | Ph-3,5-Br₂ |
| H | C(CH₃)₂CH₂SCH₃ | CF₃ | Ph-3-CH₃-4-F |
| H | C(CH₃)₂CH₂SCH₃ | CF₃ | Ph-3-F-4-CH₃ |
| H | C(CH₃)₂CH₂SCH₃ | CF₃ | Ph-2-F-5-CH₃ |
| H | C(CH₃)₂CH₂SCH₃ | CF₃ | Ph-2,4-(CH₃)₂ |
| H | C(CH₃)₂CH₂SCH₃ | CF₃ | Ph-3,4-(CH₃)₂ |
| H | C(CH₃)₂CH₂SCH₃ | CF₃ | Ph-2-F-3-CF₃ |
| H | C(CH₃)₂CH₂SCH₃ | CF₃ | Ph-3-CF₃-4-F |
| H | C(CH₃)₂CH₂SCH₃ | CF₃ | Ph-3-CF₃-4-Cl |
| H | C(CH₃)₂CH₂SCH₃ | CF₃ | Ph-2-F-4-CF₃ |
| H | C(CH₃)₂CH₂SCH₃ | CF₃ | Ph-3-F-4-CF₃ |
| H | C(CH₃)₂CH₂SCH₃ | CF₃ | Ph-2-F-5-CF₃ |
| H | C(CH₃)₂CH₂SCH₃ | CF₃ | Ph-3-F-5-CF₃ |
| H | C(CH₃)₂CH₂SCH₃ | CF₃ | Ph-2-Cl-4-CF₃ |
| H | C(CH₃)₂CH₂SCH₃ | CF₃ | Ph-3,5-(CF₃)₂ |
| H | C(CH₃)₂CH₂SCH₃ | CF₃ | Ph-3-Br-4-OCH₃ |
| H | C(CH₃)₂CH₂SCH₃ | CF₃ | Ph-3-F-4-OCHF₂ |
| H | C(CH₃)₂CH₂SCH₃ | CF₃ | Ph-3-Cl-4-OCHF₂ |
| H | C(CH₃)₂CH₂SCH₃ | CF₃ | Ph-3-Br-4-OCHF₂ |
| H | C(CH₃)₂CH₂SCH₃ | CF₃ | Ph-3-F-4-OCF₃ |
| H | C(CH₃)₂CH₂SCH₃ | CF₃ | Ph-3-Cl-4-OCF₃ |
| H | C(CH₃)₂CH₂SCH₃ | CF₃ | Ph-3-Br-4-OCF₃ |
| H | C(CH₃)₂CH₂SCH₃ | CF₃ | Ph-3-F-4-OCF₂Br |
| H | C(CH₃)₂CH₂SCH₃ | CF₃ | Ph-3-Cl-4-OCF₂Br |
| H | C(CH₃)₂CH₂SCH₃ | CF₃ | Ph-3-Br-4-OCF₂Br |
| H | C(CH₃)₂CH₂SCH₃ | CF₃ | Ph-3-F-4-OCF₂CHF₂ |
| H | C(CH₃)₂CH₂SCH₃ | CF₃ | Ph-3-Cl-4-OCF₂CHF₂ |
| H | C(CH₃)₂CH₂SCH₃ | CF₃ | Ph-3-Br-4-OCF₂CHF₂ |
| H | C(CH₃)₂CH₂SCH₃ | CF₃ | Ph-3-F-4-OCF₂CHFCl |
| H | C(CH₃)₂CH₂SCH₃ | CF₃ | Ph-3-Cl-4-OCF₂CHFCl |
| H | C(CH₃)₂CH₂SCH₃ | CF₃ | Ph-3-Br-4-OCF₂CHFCl |
| H | C(CH₃)₂CH₂SCH₃ | CF₃ | Ph-3-F-4-OCF₂CHFCF₃ |
| H | C(CH₃)₂CH₂SCH₃ | CF₃ | Ph-3-Cl-4-OCF₂CHFCF₃ |
| H | C(CH₃)₂CH₂SCH₃ | CF₃ | Ph-3-Br-4-OCF₂CHFCF₃ |
| H | C(CH₃)₂CH₂SCH₃ | CF₃ | Ph-3-F-4-OCF₂CHFOCF₃ |
| H | C(CH₃)₂CH₂SCH₃ | CF₃ | Ph-3-Cl-4-OCF₂CHFOCF₃ |
| H | C(CH₃)₂CH₂SCH₃ | CF₃ | Ph-3-Br-4-OCF₂CHFOCF₃ |
| H | C(CH₃)₂CH₂SCH₃ | CF₃ | Ph-3-CH₃-4-OCF₂CHFOCF₃ |
| H | C(CH₃)₂CH₂SCH₃ | CF₃ | Ph-3-F-4-OCF₂CHFOCF₂CF₂CF₃ |
| H | C(CH₃)₂CH₂SCH₃ | CF₃ | Ph-3-Cl-4-OCF₂CHFOCF₂CF₂CF₃ |
| H | C(CH₃)₂CH₂SCH₃ | CF₃ | Ph-3-Br-4-OCF₂CHFOCF₂CF₂CF₃ |
| H | C(CH₃)₂CH₂SCH₃ | CF₃ | Ph(-3-OCF₂O-4-) |
| H | C(CH₃)₂CH₂SCH₃ | CF₃ | Ph(-3-OCF₂CF₂O-4-) |
| H | C(CH₃)₂CH₂SCH₃ | CF₃ | Ph-3-OPh-4-F |
| H | C(CH₃)₂CH₂SCH₃ | CF₃ | Ph-3-NO₂-4-F |
| H | C(CH₃)₂CH₂SCH₃ | CF₃ | Ph-3-NO₂-4-Cl |
| H | C(CH₃)₂CH₂SCH₃ | CF₃ | Ph-2-F-5-NO₂ |
| H | C(CH₃)₂CH₂SCH₃ | CF₃ | Ph-3-CN-4-F |
| H | C(CH₃)₂CH₂SCH₃ | CF₃ | Ph-2,3,4-F₃ |
| H | C(CH₃)₂CH₂SCH₃ | CF₃ | Ph-2,3,5-F₃ |
| H | C(CH₃)₂CH₂SCH₃ | CF₃ | Ph-2,4,5-F₃ |
| H | C(CH₃)₂CH₂SCH₃ | CF₃ | Ph-3,4,5-F₃ |
| H | C(CH₃)₂CH₂SCH₃ | CF₃ | Ph-2,3-F₂-4-CH₃ |
| H | C(CH₃)₂CH₂SCH₃ | CF₃ | Ph-2,3-F₂-4-CF₃ |
| H | C(CH₃)₂CH₂SCH₃ | CF₃ | Ph-3,4-F₂-5-CF₃ |
| H | C(CH₃)₂CH₂SCH₃ | CF₃ | Ph-2-F-3-Cl-5-CF₃ |
| H | C(CH₃)₂CH₂SCH₃ | CF₃ | Ph-3,5-Cl₂-4-OCH₃ |
| H | C(CH₃)₂CH₂SCH₃ | CF₃ | 1-Naph |
| H | C(CH₃)₂CH₂SCH₃ | CF₃ | 2-Naph |
| H | C(CH₃)₂CH₂SCH₃ | CF₃ | (L-1b)Br |
| H | C(CH₃)₂CH₂SCH₃ | CF₃ | (L-1c)Cl |
| H | C(CH₃)₂CH₂SCH₃ | CF₃ | (L-1c)Br |
| H | C(CH₃)₂CH₂SCH₃ | CF₃ | (L-1c)I |
| H | C(CH₃)₂CH₂SCH₃ | CF₃ | (L-1c)CF₃ |
| H | C(CH₃)₂CH₂SCH₃ | CF₃ | (L-2b)Br |
| H | C(CH₃)₂CH₂SCH₃ | CF₃ | (L-3b)Cl |
| H | C(CH₃)₂CH₂SCH₃ | CF₃ | (L-3b)Br |
| H | C(CH₃)₂CH₂SCH₃ | CF₃ | (L-3c)F |
| H | C(CH₃)₂CH₂SCH₃ | CF₃ | (L-3c)Cl |
| H | C(CH₃)₂CH₂SCH₃ | CF₃ | (L-3c)Br |
| H | C(CH₃)₂CH₂SCH₃ | CF₃ | (L-3c)I |
| H | C(CH₃)₂CH₂SCH₃ | CF₃ | (L-3c)CF₃ |
| H | C(CH₃)₂CH₂SCH₃ | CF₃ | (L-3c)CN |
| H | C(CH₃)₂CH₂SCH₃ | CF₃ | L-3d |
| H | C(CH₃)₂CH₂SCH₃ | CF₃ | (L-4d)Cl |
| H | C(CH₃)₂CH₂SCH₃ | CF₃ | (L-4b)Br |
| H | C(CH₃)₂CH₂SCH₃ | CF₃ | (L-4b)CN |
| H | C(CH₃)₂CH₂SCH₃ | CF₃ | (L-10b)Cl |
| H | C(CH₃)₂CH₂SCH₃ | CF₃ | (L-10b)Br |
| H | C(CH₃)₂CH₂SCH₃ | CF₃ | (L-15b)CF₃ |
| H | C(CH₃)₂CH₂SCH₃ | CF₃ | (L-16a)CHF₂ |
| H | C(CH₃)₂CH₂SCH₃ | CF₃ | (L-16a)CF₂Br |
| H | C(CH₃)₂CH₂SCH₃ | CF₃ | (L-17a)Cl |
| H | C(CH₃)₂CH₂SCH₃ | CF₃ | (L-21b)Cl |
| H | C(CH₃)₂CH₂SCH₃ | CF₃ | (L-21b)Br |
| H | C(CH₃)₂CH₂SCH₃ | CF₃ | (L-21b)I |
| H | C(CH₃)₂CH₂SCH₃ | CF₃ | (L-21b)CF₃ |
| H | C(CH₃)₂CH₂SCH₃ | CF₃ | (L-22b)Cl |
| H | C(CH₃)₂CH₂SCH₃ | CF₃ | (L-22b)Br |
| H | C(CH₃)₂CH₂SCH₃ | CF₃ | (L-23b)Cl |
| H | C(CH₃)₂CH₂SCH₃ | CF₃ | (L-23b)Br |
| H | C(CH₃)₂CH₂SCH₃ | CF₃ | (L-23c)Cl |
| H | C(CH₃)₂CH₂SCH₃ | CF₃ | (L-23c)Br |
| H | C(CH₃)₂CH₂SCH₃ | CF₃ | (L-31a)Cl |
| H | C(CH₃)₂CH₂SCH₃ | CF₃ | (L-31a)Br |
| H | C(CH₃)₂CH₂SCH₃ | CF₃ | (L-45c)F |
| H | C(CH₃)₂CH₂SCH₃ | CF₃ | (L-45c)Cl |
| H | C(CH₃)₂CH₂SCH₃ | CF₃ | (L-45c)Br |
| H | C(CH₃)₂CH₂SCH₃ | CF₃ | (L-45c)I |
| H | C(CH₃)₂CH₂SCH₃ | CF₃ | (L-45c)CF₃ |
| H | C(CH₃)₂CH₂SCH₃ | CF₃ | L-45e |
| H | C(CH₃)₂CH₂SCH₃ | CF₃ | L-45f |
| H | C(CH₃)₂CH₂SCH₃ | CF₃ | (L-46c)F |
| H | C(CH₃)₂CH₂SCH₃ | CF₃ | (L-46c)Cl |
| H | C(CH₃)₂CH₂SCH₃ | CF₃ | (L-46c)Br |
| H | C(CH₃)₂CH₂SCH₃ | CF₃ | (L-46c)I |
| H | C(CH₃)₂CH₂SCH₃ | CF₃ | (L-46c)CF₃ |
| H | C(CH₃)₂CH₂SCH₃ | CF₃ | (L-46c)OCH₂CF₃ |
| H | C(CH₃)₂CH₂SCH₃ | CF₃ | (L-46c)OCH(CF₃)₂ |
| H | C(CH₃)₂CH₂SCH₃ | CF₃ | L-46d |
| H | C(CH₃)₂CH₂SCH₃ | CF₃ | L-47a |
| H | C(CH₃)₂CH₂SCH₃ | CF₃ | L-47d |
| H | C(CH₃)₂CH₂SCH₃ | CF₃ | (L-48b)Br |
| H | C(CH₃)₂CH₂SCH₃ | CF₃ | (L-50b)Cl |
| H | C(CH₃)₂CH₂SCH₃ | CF₃ | (L-50b)Br |
| H | C(CH₃)₂CH₂SCH₃ | CF₃ | (L-51b)Cl |
| H | C(CH₃)₂CH₂SCH₃ | CF₂Cl | Ph-4-Cl |
| H | C(CH₃)₂CH₂SCH₃ | CF₂Cl | Ph-4-Br |
| H | C(CH₃)₂CH₂SCH₃ | CF₂Cl | Ph-4-CF₃ |
| H | C(CH₃)₂CH₂SCH₃ | CF₂Cl | Ph-4-OCF₃ |
| H | C(CH₃)₂CH₂SCH₃ | CF₂Cl | Ph-4-OCF₂Br |
| H | C(CH₃)₂CH₂SCH₃ | CF₂Cl | Ph-4-OCF₂CHFCF₃ |
| H | C(CH₃)₂CH₂SCH₃ | CF₂Cl | Ph-4-OCF₂CHFOCF₃ |
| H | C(CH₃)₂CH₂SCH₃ | CF₂Cl | Ph-4-OCF₂CHFOCF₂CF₂CF₃ |
| H | C(CH₃)₂CH₂SCH₃ | CF₂Cl | Ph-4-O(L-45g) |
| H | C(CH₃)₂CH₂SCH₃ | CF₂Cl | Ph-3,4-Cl₂ |
| H | C(CH₃)₂CH₂SCH₃ | CF₂Cl | Ph(-3-OCF₂O-4-) |
| H | C(CH₃)₂CH₂SCH₃ | CF₂Cl | Ph(-3-OCF₂CF₂O-4-) |
| H | C(CH₃)₂CH₂SCH₃ | CF₂Cl | (L-45c)CF₃ |
| H | C(CH₃)₂CH₂SCH₃ | CF₂Cl | L-45f |
| H | C(CH₃)₂CH₂SCH₃ | CF₂Cl | (L-46c)CF₃ |
| H | C(CH₃)₂CH₂SCH₃ | CF₂Cl | L-46d |
| H | C(CH₃)₂CH₂SCH₃ | CFCl₂ | Ph-4-OCF₃ |
| H | C(CH₃)₂CH₂SCH₃ | CF₂Br | Ph-4-Cl |
| H | C(CH₃)₂CH₂SCH₃ | CF₂Br | Ph-4-Br |
| H | C(CH₃)₂CH₂SCH₃ | CF₂Br | Ph-4-CF₃ |
| H | C(CH₃)₂CH₂SCH₃ | CF₂Br | Ph-4-OCF₃ |
| H | C(CH₃)₂CH₂SCH₃ | CF₂Br | Ph-4-OCF₂Br |
| H | C(CH₃)₂CH₂SCH₃ | CF₂Br | Ph-4-OCF₂CHFCF₃ |
| H | C(CH₃)₂CH₂SCH₃ | CF₂Br | Ph-4-OCF₂CHFOCF₃ |
| H | C(CH₃)₂CH₂SCH₃ | CF₂Br | Ph-4-OCF₂CHFOCF₂CF₂CF₃ |
| H | C(CH₃)₂CH₂SCH₃ | CF₂Br | Ph-4-O(L-45g) |
| H | C(CH₃)₂CH₂SCH₃ | CF₂Br | Ph-3,4-Cl₂ |
| H | C(CH₃)₂CH₂SCH₃ | CF₂Br | Ph(-3-OCF₂O-4-) |
| H | C(CH₃)₂CH₂SCH₃ | CF₂Br | Ph(-3-OCF₂CF₂O-4-) |
| H | C(CH₃)₂CH₂SCH₃ | CF₂Br | (L-45c)CF₃ |
| H | C(CH₃)₂CH₂SCH₃ | CF₂Br | L-45f |
| H | C(CH₃)₂CH₂SCH₃ | CF₂Br | (L-46c)CF₃ |
| H | C(CH₃)₂CH₂SCH₃ | CF₂Br | L-46d |
| H | C(CH₃)₂CH₂SCH₃ | CFClBr | (L-45c)CF₃ |
| H | C(CH₃)₂CH₂SCH₃ | CFBr₂ | Ph-4-Br |
| H | C(CH₃)₂CH₂SCH₃ | CF₂CHF₂ | Ph-4-CF₃ |
| H | C(CH₃)₂CH₂SCH₃ | CF₂CF₃ | Ph-4-Cl |
| H | C(CH₃)₂CH₂SCH₃ | CF₂CF₃ | Ph-4-Br |
| H | C(CH₃)₂CH₂SCH₃ | CF₂CF₃ | Ph-4-CF₃ |
| H | C(CH₃)₂CH₂SCH₃ | CF₂CF₃ | Ph-4-OCF₃ |
| H | C(CH₃)₂CH₂SCH₃ | CF₂CF₃ | Ph-4-OCF₂Br |
| H | C(CH₃)₂CH₂SCH₃ | CF₂CF₃ | Ph-4-OCF₂CHFCF₃ |
| H | C(CH₃)₂CH₂SCH₃ | CF₂CF₃ | Ph-4-OCF₂CHFOCF₃ |
| H | C(CH₃)₂CH₂SCH₃ | CF₂CF₃ | Ph-4-OCF₂CHFOCF₂CF₂CF₃ |
| H | C(CH₃)₂CH₂SCH₃ | CF₂CF₃ | Ph-4-O(L-45g) |
| H | C(CH₃)₂CH₂SCH₃ | CF₂CF₃ | Ph-3,4-Cl₂ |
| H | C(CH₃)₂CH₂SCH₃ | CF₂CF₃ | Ph(-3-OCF₂O-4-) |
| H | C(CH₃)₂CH₂SCH₃ | CF₂CF₃ | Ph(-3-OCF₂CF₂O-4-) |
| H | C(CH₃)₂CH₂SCH₃ | CF₂CF₃ | (L-45c)CF₃ |
| H | C(CH₃)₂CH₂SCH₃ | CF₂CF₃ | L-45f |
| H | C(CH₃)₂CH₂SCH₃ | CF₂CF₃ | (L-46c)CF₃ |
| H | C(CH₃)₂CH₂SCH₃ | CF₂CF₃ | L-46d |
| H | C(CH₃)₂CH₂SCH₃ | CF₂CF₂Cl | Ph-4-OCF₃ |
| H | C(CH₃)₂CH₂SCH₃ | CFClCF₃ | (L-45c)CF₃ |
| H | C(CH₃)₂CH₂SCH₃ | CFClCF₂Cl | Ph-4-Br |
| H | C(CH₃)₂CH₂SCH₃ | CF₂CF₂Br | Ph-4-CF₃ |
| H | C(CH₃)₂CH₂SCH₃ | CFBrCF₃ | Ph-4-OCF₃ |
| H | C(CH₃)₂CH₂SCH₃ | CF₂CHFCF₃ | (L-45c)CF₃ |
| H | C(CH₃)₂CH₂SCH₃ | CF₂CF₂CF₃ | Ph-4-Cl |
| H | C(CH₃)₂CH₂SCH₃ | CF₂CF₂CF₃ | Ph-4-Br |
| H | C(CH₃)₂CH₂SCH₃ | CF₂CF₂CF₃ | Ph-4-CF₃ |
| H | C(CH₃)₂CH₂SCH₃ | CF₂CF₂CF₃ | Ph-4-OCF₃ |
| H | C(CH₃)₂CH₂SCH₃ | CF₂CF₂CF₃ | Ph-4-OCF₂Br |
| H | C(CH₃)₂CH₂SCH₃ | CF₂CF₂CF₃ | Ph-4-OCF₂CHFCF₃ |
| H | C(CH₃)₂CH₂SCH₃ | CF₂CF₂CF₃ | Ph-4-OCF₂CHFOCF₃ |
| H | C(CH₃)₂CH₂SCH₃ | CF₂CF₂CF₃ | Ph-4-OCF₂CHFOCF₂CF₂CF₃ |
| H | C(CH₃)₂CH₂SCH₃ | CF₂CF₂CF₃ | Ph-4-O(L-45g) |
| H | C(CH₃)₂CH₂SCH₃ | CF₂CF₂CF₃ | Ph-3,4-Cl₂ |
| H | C(CH₃)₂CH₂SCH₃ | CF₂CF₂CF₃ | Ph(-3-OCF₂O-4-) |
| H | C(CH₃)₂CH₂SCH₃ | CF₂CF₂CF₃ | Ph(-3-OCF₂CF₂O-4-) |
| H | C(CH₃)₂CH₂SCH₃ | CF₂CF₂CF₃ | (L-45c)CF₃ |
| H | C(CH₃)₂CH₂SCH₃ | CF₂CF₂CF₃ | L-45f |
| H | C(CH₃)₂CH₂SCH₃ | CF₂CF₂CF₃ | (L-46c)CF₃ |
| H | C(CH₃)₂CH₂SCH₃ | CF₂CF₂CF₃ | L-46d |
| H | C(CH₃)₂CH₂SCH₃ | CF(CF₃)₂ | Ph-4-Br |
| H | C(CH₃)₂CH₂SCH₃ | CF₂CF₂CF₂CF₃ | Ph-4-CF₃ |
| H | C(CH₃)₂CH₂SCH₃ | T-1 | Ph-4-OCF₃ |
| H | C(CH₃)₂CH₂SCH₃ | T-2 | (L-45c)CF₃ |
| H | C(CH₃)₂CH₂SCH₃ | CH₂OCH₃ | Ph-4-Br |
| H | C(CH₃)₂CH₂SCH₃ | CH₂OEt | Ph-4-CF₃ |
| H | C(CH₃)₂CH₂SCH₃ | CH₂OCH₂CF₃ | Ph-4-OCF₃ |
| H | C(CH₃)₂CH₂SCH₃ | CH₂OCH(CF₃)₂ | (L-45c)CF₃ |
| H | C(CH₃)₂CH₂SCH₃ | CF₂OCH₃ | Ph-4-Cl |
| H | C(CH₃)₂CH₂SCH₃ | CF₂OCH₃ | Ph-4-Br |
| H | C(CH₃)₂CH₂SCH₃ | CF₂OCH₃ | Ph-4-CF₃ |
| H | C(CH₃)₂CH₂SCH₃ | CF₂OCH₃ | Ph-4-OCF₃ |
| H | C(CH₃)₂CH₂SCH₃ | CF₂OCH₃ | Ph-4-OCF₂Br |
| H | C(CH₃)₂CH₂SCH₃ | CF₂OCH₃ | Ph-4-OCf₂CHFCF₃ |
| H | C(CH₃)₂CH₂SCH₃ | CF₂OCH₃ | Ph-4-OCF₂CHFOCF₃ |
| H | C(CH₃)₂CH₂SCH₃ | CF₂OCH₃ | Ph-4-OCF₂CHFOCF₂CF₂CF₃ |
| H | C(CH₃)₂CH₂SCH₃ | CF₂OCH₃ | Ph-4-O(L-45g) |
| H | C(CH₃)₂CH₂SCH₃ | CF₂OCH₃ | Ph-3,4-Cl₂ |
| H | C(CH₃)₂CH₂SCH₃ | CF₂OCH₃ | Ph(-3-OCF₂O-4-) |
| H | C(CH₃)₂CH₂SCH₃ | CF₂OCH₃ | Ph(-3-OCF₂CF₂O-4-) |
| H | C(CH₃)₂CH₂SCH₃ | CF₂OCH₃ | (L-45c)CF₃ |
| H | C(CH₃)₂CH₂SCH₃ | CF₂OCH₃ | L-45f |
| H | C(CH₃)₂CH₂SCH₃ | CF₂OCH₃ | (L-46c)CF₃ |
| H | C(CH₃)₂CH₂SCH₃ | CF₂OCH₃ | L-46d |
| H | C(CH₃)₂CH₂SCH₃ | CH₂SCH₃ | Ph-4-Br |
| H | C(CH₃)₂CH₂SCH₃ | CH₂SO₂CH₃ | Ph-4-CF₃ |
| H | C(CH₃)₂CH₂SCH₃ | CH₂SCF₃ | Ph-4-OCF₃ |
| H | C(CH₃)₂CH₂SCH₃ | CF₂SCH₃ | Ph-4-Cl |
| H | C(CH₃)₂CH₂SCH₃ | CF₂SCH₃ | Ph-4-Br |
| H | C(CH₃)₂CH₂SCH₃ | CF₂SCH₃ | Ph-4-CF₃ |
| H | C(CH₃)₂CH₂SCH₃ | CF₂SCH₃ | Ph-4-OCF₃ |
| H | C(CH₃)₂CH₂SCH₃ | CF₂SCH₃ | Ph-4-OCF₂Br |
| H | C(CH₃)₂CH₂SCH₃ | CF₂SCH₃ | Ph-4-OCF₂CHFCF₃ |
| H | C(CH₃)₂CH₂SCH₃ | CF₂SCH₃ | Ph-4-OCF₂CHFOCF₃ |
| H | C(CH₃)₂CH₂SCH₃ | CF₂SCH₃ | Ph-4-OCF₂CHFOCF₂CF₂CF₃ |
| H | C(CH₃)₂CH₂SCH₃ | CF₂SCH₃ | Ph-4-O(L-45g) |
| H | C(CH₃)₂CH₂SCH₃ | CF₂SCH₃ | Ph-3,4-Cl₂ |
| H | C(CH₃)₂CH₂SCH₃ | CF₂SCH₃ | Ph(-3-OCF₂O-4-) |
| H | C(CH₃)₂CH₂SCH₃ | CF₂SCH₃ | Ph(-3-OCF₂CF₂O-4-) |
| H | C(CH₃)₂CH₂SCH₃ | CF₂SCH₃ | (L-45c)CF₃ |
| H | C(CH₃)₂CH₂SCH₃ | CF₂SCH₃ | L-45f |
| H | C(CH₃)₂CH₂SCH₃ | CF₂SCH₃ | (L-46c)CF₃ |
| H | C(CH₃)₂CH₂SCH₃ | CF₂SCH₃ | L-46d |
| H | C(CH₃)₂CH₂SCH₃ | CF₂SEt | (L-45c)CF₃ |
| H | C(CH₃)₂CH₂SCH₃ | CF₂SPr-n | Ph-4-Br |
| H | C(CH₃)₂CH₂SCH₃ | CF₂SPr-i | Ph-4-CF₃ |
| H | C(CH₃)₂CH₂SCH₃ | CF₂SCH₂Ph | Ph-4-OCF₃ |
| H | C(CH₃)₂CH₂SCH₃ | CF₂SPh | (L-45c)CF₃ |
| H | C(CH₃)₂CH₂SCH₃ | CF₂C(O)OEt | Ph-4-Br |
| H | C(CH₃)₂CH₂SCH₃ | CF₂SO₂N(CH₃)₂ | Ph-4-CF₃ |
| H | C(CH₃)₂CH₂SCH₃ | C(O)OEt | Ph-4-OCF₃ |
| H | C(CH₃)₂CH₂SCH₃ | C(O)SEt | (L-45c)CF₃ |
| H | C(CH₃)₂CH₂S(O)CH₃ | CH₃ | Ph-4-Br |
| H | C(CH₃)₂CH₂S(O)CH₃ | CH₃ | Ph-4-CF₃ |
| H | C(CH₃)₂CH₂S(O)CH₃ | CH₃ | Ph-4-OCF₃ |
| H | C(CH₃)₂CH₂S(O)CH₃ | CH₃ | (L-45c)CF₃ |
| H | C(CH₃)₂CH₂S(O)CH₃ | CF₃ | CH₂O(Ph-4-Cl) |
| H | C(CH₃)₂CH₂S(O)CH₃ | CF₃ | CH₂O(Ph-4-Br) |
| H | C(CH₃)₂CH₂S(O)CH₃ | CF₃ | CH₂O(Ph-4-CF₃) |
| H | C(CH₃)₂CH₂S(O)CH₃ | CF₃ | CH₂O(Ph-4-OCF₃) |
| H | C(CH₃)₂CH₂S(O)CH₃ | CF₃ | CF₂O(Ph-4-Cl) |
| H | C(CH₃)₂CH₂S(O)CH₃ | CF₃ | CF₂O(Ph-4-Br) |
| H | C(CH₃)₂CH₂S(O)CH₃ | CF₃ | CF₂O(Ph-4-CF₃) |
| H | C(CH₃)₂CH₂S(O)CH₃ | CF₃ | CF₂O(Ph-4-OCF₃) |
| H | C(CH₃)₂CH₂S(O)CH₃ | CF₃ | CH═CH(Ph-4-F) |
| H | C(CH₃)₂CH₂S(O)CH₃ | CF₃ | CH═CH(Ph-4-Cl) |
| H | C(CH₃)₂CH₂S(O)CH₃ | CF₃ | CH═CH(Ph-4-Br) |
| H | C(CH₃)₂CH₂S(O)CH₃ | CF₃ | CH═CH(Ph-4-CF₃) |
| H | C(CH₃)₂CH₂S(O)CH₃ | CF₃ | CH═CH(Ph-4-OCF₃) |
| H | C(CH₃)₂CH₂S(O)CH₃ | CF₃ | CH═CH(Ph-4-SCH₃) |
| H | C(CH₃)₂CH₂S(O)CH₃ | CF₃ | CH═CH(Ph-4-SO₂CH₃) |
| H | C(CH₃)₂CH₂S(O)CH₃ | CF₃ | CH═CH(Ph-3,4-Cl₂) |
| H | C(CH₃)₂CH₂S(O)CH₃ | CF₃ | Ph-4-F |
| H | C(CH₃)₂CH₂S(O)CH₃ | CF₃ | Ph-2-Cl |
| H | C(CH₃)₂CH₂S(O)CH₃ | CF₃ | Ph-3-Cl |
| H | C(CH₃)₂CH₂S(O)CH₃ | CF₃ | Ph-4-Cl |
| H | C(CH₃)₂CH₂S(O)CH₃ | CF₃ | Ph-4-Br |
| H | C(CH₃)₂CH₂S(O)CH₃ | CF₃ | Ph-4-I |
| H | C(CH₃)₂CH₂S(O)CH₃ | CF₃ | Ph-4-CF₃ |
| H | C(CH₃)₂CH₂S(O)CH₃ | CF₃ | Ph-4-OCHF₂ |
| H | C(CH₃)₂CH₂S(O)CH₃ | CF₃ | Ph-4-OCF₃ |
| H | C(CH₃)₂CH₂S(O)CH₃(−) | CF₃ | Ph-4-OCF₃ |
| H | C(CH₃)₂CH₂S(O)CH₃(+) | CF₃ | Ph-4-OCF₃ |
| H | C(CH₃)₂CH₂S(O)CH₃ | CF₃ | Ph-4-OCF₂Br |
| H | C(CH₃)₂CH₂S(O)CH₃ | CF₃ | Ph-4-OCH₂CF₃ |
| H | C(CH₃)₂CH₂S(O)CH₃ | CF₃ | Ph-4-OCF₂CHF₂ |
| H | C(CH₃)₂CH₂S(O)CH₃ | CF₃ | Ph-4-OCF₂CHFCl |
| H | C(CH₃)₂CH₂S(O)CH₃ | CF₃ | Ph-4-OCF₂CHFCF₃ |
| H | C(CH₃)₂CH₂S(O)CH₃ | CF₃ | Ph-4-OCF₂CHFOCF₃ |
| H | C(CH₃)₂CH₂S(O)CH₃ | CF₃ | Ph-4-OCF₂CHFOCF₂CF₂CF₃ |
| H | C(CH₃)₂CH₂S(O)CH₃ | CF₃ | Ph-4-OSO₂CH₃ |
| H | C(CH₃)₂CH₂S(O)CH₃ | CF₃ | Ph-4-OSO₂CF₃ |
| H | C(CH₃)₂CH₂S(O)CH₃ | CF₃ | Ph-4-O(L-45e) |
| H | C(CH₃)₂CH₂S(O)CH₃ | CF₃ | Ph-2,4-F₂ |
| H | C(CH₃)₂CH₂S(O)CH₃ | CF₃ | Ph-3,4-F₂ |
| H | C(CH₃)₂CH₂S(O)CH₃ | CF₃ | Ph-3-Cl-4-F |
| H | C(CH₃)₂CH₂S(O)CH₃ | CF₃ | Ph-2-F-4-Cl |
| H | C(CH₃)₂CH₂S(O)CH₃ | CF₃ | Ph-3-F-4-Cl |
| H | C(CH₃)₂CH₂S(O)CH₃ | CF₃ | Ph-2,4-Cl₂ |
| H | C(CH₃)₂CH₂S(O)CH₃ | CF₃ | Ph-3,4-Cl₂ |
| H | C(CH₃)₂CH₂S(O)CH₃ | CF₃ | Ph-3,5-Cl₂ |
| H | C(CH₃)₂CH₂S(O)CH₃ | CF₃ | Ph-3-Br-4-F |
| H | C(CH₃)₂CH₂S(O)CH₃ | CF₃ | Ph-2-F-4-Br |
| H | C(CH₃)₂CH₂S(O)CH₃ | CF₃ | Ph-3,4-Br₂ |
| H | C(CH₃)₂CH₂S(O)CH₃ | CF₃ | Ph-3-CH₃-4-F |
| H | C(CH₃)₂CH₂S(O)CH₃ | CF₃ | Ph-3-CF₃-4-F |
| H | C(CH₃)₂CH₂S(O)CH₃ | CF₃ | Ph-3-CF₃-4-Cl |
| H | C(CH₃)₂CH₂S(O)CH₃ | CF₃ | Ph-2-F-4-CF₃ |
| H | C(CH₃)₂CH₂S(O)CH₃ | CF₃ | Ph-3-F-4-CF₃ |
| H | C(CH₃)₂CH₂S(O)CH₃ | CF₃ | Ph-2-Cl-4-CF₃ |
| H | C(CH₃)₂CH₂S(O)CH₃ | CF₃ | Ph-3-F-4-OCHF₂ |
| H | C(CH₃)₂CH₂S(O)CH₃ | CF₃ | Ph-3-Cl-4-OCHF₂ |
| H | C(CH₃)₂CH₂S(O)CH₃ | CF₃ | Ph-3-Br-4-OCHF₂ |
| H | C(CH₃)₂CH₂S(O)CH₃ | CF₃ | Ph-3-F-4-OCF₃ |
| H | C(CH₃)₂CH₂S(O)CH₃ | CF₃ | Ph-3-Cl-4-OCF₃ |
| H | C(CH₃)₂CH₂S(O)CH₃ | CF₃ | Ph-3-Br-4-OCF₃ |
| H | C(CH₃)₂CH₂S(O)CH₃ | CF₃ | Ph-3-F-4-OCF₂Br |
| H | C(CH₃)₂CH₂S(O)CH₃ | CF₃ | Ph-3-Cl-4-OCF₂Br |
| H | C(CH₃)₂CH₂S(O)CH₃ | CF₃ | Ph-3-Br-4-OCF₂Br |
| H | C(CH₃)₂CH₂S(O)CH₃ | CF₃ | Ph-3-F-4-OCF₂CHF₂ |
| H | C(CH₃)₂CH₂S(O)CH₃ | CF₃ | Ph-3-Cl-4-OCF₂CHF₂ |
| H | C(CH₃)₂CH₂S(O)CH₃ | CF₃ | Ph-3-Br-4-OCF₂CHF₂ |
| H | C(CH₃)₂CH₂S(O)CH₃ | CF₃ | Ph-3-F-4-OCF₂CHFCl |
| H | C(CH₃)₂CH₂S(O)CH₃ | CF₃ | Ph-3-Cl-4-OCF₂CHFCl |
| H | C(CH₃)₂CH₂S(O)CH₃ | CF₃ | Ph-3-Br-4-OCF₂CHFCl |
| H | C(CH₃)₂CH₂S(O)CH₃ | CF₃ | Ph-3-F-4-OCF₂CHFCF₃ |
| H | C(CH₃)₂CH₂S(O)CH₃ | CF₃ | Ph-3-Cl-4-OCF₂CHFCF₃ |
| H | C(CH₃)₂CH₂S(O)CH₃ | CF₃ | Ph-3-Br-4-OCF₂CHFCF₃ |
| H | C(CH₃)₂CH₂S(O)CH₃ | CF₃ | Ph-3-F-4-OCF₂CHFOCF₃ |
| H | C(CH₃)₂CH₂S(O)CH₃ | CF₃ | Ph-3-Cl-4-OCF₂CHFOCF₃ |
| H | C(CH₃)₂CH₂S(O)CH₃ | CF₃ | Ph-3-Br-4-OCF₂CHFOCF₃ |
| H | C(CH₃)₂CH₂S(O)CH₃ | CF₃ | Ph-3-CH₃-4-OCF₂CHFOCF₃ |
| H | C(CH₃)₂CH₂S(O)CH₃ | CF₃ | Ph-3-F-4-OCF₂CHFOCF₂CF₂CF₃ |
| H | C(CH₃)₂CH₂S(O)CH₃ | CF₃ | Ph-3-Cl-4-OCF₂CHFOCF₂CF₂CF₃ |
| H | C(CH₃)₂CH₂S(O)CH₃ | CF₃ | Ph-3-Br-4-OCF₂CHFOCF₂CF₂CF₃ |
| H | C(CH₃)₂CH₂S(O)CH₃ | CF₃ | Ph(-3-OCF₂O-4-) |
| H | C(CH₃)₂CH₂S(O)CH₃ | CF₃ | Ph(-3-OCF₂CF₂O-4-) |
| H | C(CH₃)₂CH₂S(O)CH₃ | CF₃ | Ph-3,4-F₂-5-CF₃ |
| H | C(CH₃)₂CH₂S(O)CH₃ | CF₃ | 2-Naph |
| H | C(CH₃)₂CH₂S(O)CH₃ | CF₃ | (L-1b)Br |
| H | C(CH₃)₂CH₂S(O)CH₃ | CF₃ | (L-1c)Cl |
| H | C(CH₃)₂CH₂S(O)CH₃ | CF₃ | (L-1c)Br |
| H | C(CH₃)₂CH₂S(O)CH₃ | CF₃ | (L-1c)CF₃ |
| H | C(CH₃)₂CH₂S(O)CH₃ | CF₃ | (L-2b)Br |
| H | C(CH₃)₂CH₂S(O)CH₃ | CF₃ | (L-3b)Cl |
| H | C(CH₃)₂CH₂S(O)CH₃ | CF₃ | (L-3b)Br |
| H | C(CH₃)₂CH₂S(O)CH₃ | CF₃ | (L-3c)Cl |
| H | C(CH₃)₂CH₂S(O)CH₃ | CF₃ | (L-3c)Br |
| H | C(CH₃)₂CH₂S(O)CH₃ | CF₃ | (L-3c)CF₃ |
| H | C(CH₃)₂CH₂S(O)CH₃ | CF₃ | L-3d |
| H | C(CH₃)₂CH₂S(O)CH₃ | CF₃ | (L-4b)Cl |
| H | C(CH₃)₂CH₂S(O)CH₃ | CF₃ | (L-4b)Br |
| H | C(CH₃)₂CH₂S(O)CH₃ | CF₃ | (L-10b)Cl |
| H | C(CH₃)₂CH₂S(O)CH₃ | CF₃ | (L-10b)Br |
| H | C(CH₃)₂CH₂S(O)CH₃ | CF₃ | (L-15b)CF₃ |
| H | C(CH₃)₂CH₂S(O)CH₃ | CF₃ | (L-16a)CHF₂ |
| H | C(CH₃)₂CH₂S(O)CH₃ | CF₃ | (L-16a)CF₂Br |
| H | C(CH₃)₂CH₂S(O)CH₃ | CF₃ | (L-17a)Cl |
| H | C(CH₃)₂CH₂S(O)CH₃ | CF₃ | (L-21b)Cl |
| H | C(CH₃)₂CH₂S(O)CH₃ | CF₃ | (L-21b)Br |
| H | C(CH₃)₂CH₂S(O)CH₃ | CF₃ | (L-21b)CF₃ |
| H | C(CH₃)₂CH₂S(O)CH₃ | CF₃ | (L-22b)Cl |
| H | C(CH₃)₂CH₂S(O)CH₃ | CF₃ | (L-22b)Br |
| H | C(CH₃)₂CH₂S(O)CH₃ | CF₃ | (L-23b)Cl |
| H | C(CH₃)₂CH₂S(O)CH₃ | CF₃ | (L-23b)Br |
| H | C(CH₃)₂CH₂S(O)CH₃ | CF₃ | (L-23c)Cl |
| H | C(CH₃)₂CH₂S(O)CH₃ | CF₃ | (L-23c)Br |
| H | C(CH₃)₂CH₂S(O)CH₃ | CF₃ | (L-31a)Cl |
| H | C(CH₃)₂CH₂S(O)CH₃ | CF₃ | (L-31a)Br |
| H | C(CH₃)₂CH₂S(O)CH₃ | CF₃ | (L-45c)F |
| H | C(CH₃)₂CH₂S(O)CH₃ | CF₃ | (L-45c)Cl |
| H | C(CH₃)₂CH₂S(O)CH₃ | CF₃ | (L-45c)Br |
| H | C(CH₃)₂CH₂S(O)CH₃ | CF₃ | (L-45c)CF₃ |
| H | C(CH₃)₂CH₂S(O)CH₃ | CF₃ | L-45e |
| H | C(CH₃)₂CH₂S(O)CH₃ | CF₃ | L-45f |
| H | C(CH₃)₂CH₂S(O)CH₃ | CF₃ | (L-46c)F |
| H | C(CH₃)₂CH₂S(O)CH₃ | CF₃ | (L-46c)Cl |
| H | C(CH₃)₂CH₂S(O)CH₃ | CF₃ | (L-46c)Br |
| H | C(CH₃)₂CH₂S(O)CH₃ | CF₃ | (L-46c)CF₃ |
| H | C(CH₃)₂CH₂S(O)CH₃ | CF₃ | L-46d |
| H | C(CH₃)₂CH₂S(O)CH₃ | CF₃ | L-47a |
| H | C(CH₃)₂CH₂S(O)CH₃ | CF₃ | L-47d |
| H | C(CH₃)₂CH₂S(O)CH₃ | CF₃ | (L-48b)Br |
| H | C(CH₃)₂CH₂S(O)CH₃ | CF₃ | (L-50b)Cl |
| H | C(CH₃)₂CH₂S(O)CH₃ | CF₃ | (L-50b)Br |
| H | C(CH₃)₂CH₂S(O)CH₃ | CF₃ | (L-51b)Cl |
| H | C(CH₃)₂CH₂SO₂CH₃ | CH₃ | CF₂O(Ph-4-Cl) |
| H | C(CH₃)₂CH₂SO₂CH₃ | CH₃ | CH═CH(Ph-4-Cl) |
| H | C(CH₃)₂CH₂SO₂CH₃ | CH₃ | CH═CH(Ph-4-OCF₃) |
| H | C(CH₃)₂CH₂SO₂CH₃ | CH₃ | CH═CH(Ph-3,4-Cl₂) |
| H | C(CH₃)₂CH₂SO₂CH₃ | CH₃ | Ph-4-F |
| H | C(CH₃)₂CH₂SO₂CH₃ | CH₃ | Ph-4-Cl |
| H | C(CH₃)₂CH₂SO₂CH₃ | CH₃ | Ph-4-Br |
| H | C(CH₃)₂CH₂SO₂CH₃ | CH₃ | Ph-4-I |
| H | C(CH₃)₂CH₂SO₂CH₃ | CH₃ | Ph-4-CF₃ |
| H | C(CH₃)₂CH₂SO₂CH₃ | CH₃ | Ph-4-OCHF₂ |
| H | C(CH₃)₂CH₂SO₂CH₃ | CH₃ | Ph-4-OCF₃ |
| H | C(CH₃)₂CH₂SO₂CH₃ | CH₃ | Ph-4-OCF₂Br |
| H | C(CH₃)₂CH₂SO₂CH₃ | CH₃ | Ph-4-OCF₂CHF₂ |
| H | C(CH₃)₂CH₂SO₂CH₃ | CH₃ | Ph-4-OCF₂CHFCl |
| H | C(CH₃)₂CH₂SO₂CH₃ | CH₃ | Ph-4-OCF₂CHFCF₃ |
| H | C(CH₃)₂CH₂SO₂CH₃ | CH₃ | Ph-4-OCF₂CHFOCF₃ |
| H | C(CH₃)₂CH₂SO₂CH₃ | CH₃ | Ph-4-OCF₂CHFOCF₂CF₂CF₃ |
| H | C(CH₃)₂CH₂SO₂CH₃ | CH₃ | Ph-4-O(L-45g) |
| H | C(CH₃)₂CH₂SO₂CH₃ | CH₃ | Ph-3,4-Cl₂ |
| H | C(CH₃)₂CH₂SO₂CH₃ | CH₃ | Ph(-3-OCF₂O-4-) |
| H | C(CH₃)₂CH₂SO₂CH₃ | CH₃ | Ph(-3-OCF₂CF₂O-4-) |
| H | C(CH₃)₂CH₂SO₂CH₃ | CH₃ | (L-45c)Cl |
| H | C(CH₃)₂CH₂SO₂CH₃ | CH₃ | (L-45c)Br |
| H | C(CH₃)₂CH₂SO₂CH₃ | CH₃ | (L-45c)CF₃ |
| H | C(CH₃)₂CH₂SO₂CH₃ | CH₃ | L-45e |
| H | C(CH₃)₂CH₂SO₂CH₃ | CH₃ | L-45f |
| H | C(CH₃)₂CH₂SO₂CH₃ | CH₃ | (L-46c)Cl |
| H | C(CH₃)₂CH₂SO₂CH₃ | CH₃ | (L-46c)Br |
| H | C(CH₃)₂CH₂SO₂CH₃ | CH₃ | (L-46c)CF₃ |
| H | C(CH₃)₂CH₂SO₂CH₃ | CH₃ | L-46d |
| H | C(CH₃)₂CH₂SO₂CH₃ | Et | Ph-4-Cl |
| H | C(CH₃)₂CH₂SO₂CH₃ | Et | Ph-4-Br |
| H | C(CH₃)₂CH₂SO₂CH₃ | Et | Ph-4-CF₃ |
| H | C(CH₃)₂CH₂SO₂CH₃ | Et | Ph-4-OCF₃ |
| H | C(CH₃)₂CH₂SO₂CH₃ | Et | Ph-4-OCF₂Br |
| H | C(CH₃)₂CH₂SO₂CH₃ | Et | Ph-4-OCF₂CHFCF₃ |
| H | C(CH₃)₂CH₂SO₂CH₃ | Et | Ph-4-OCF₂CHFOCF₃ |
| H | C(CH₃)₂CH₂SO₂CH₃ | Et | Ph-4-OCF₂CHFOCF₂CF₂CF₃ |
| H | C(CH₃)₂CH₂SO₂CH₃ | Et | Ph-4-O(L-45g) |
| H | C(CH₃)₂CH₂SO₂CH₃ | Et | Ph-3,4-Cl₂ |
| H | C(CH₃)₂CH₂SO₂CH₃ | Et | Ph(-3-OCF₂O-4-) |
| H | C(CH₃)₂CH₂SO₂CH₃ | Et | Ph(-3-OCF₂CF₂O-4-) |
| H | C(CH₃)₂CH₂SO₂CH₃ | Et | (L-45c)CF₃ |
| H | C(CH₃)₂CH₂SO₂CH₃ | n-Pr | Ph-4-Br |
| H | C(CH₃)₂CH₂SO₂CH₃ | n-Pr | Ph-4-CF₃ |
| H | C(CH₃)₂CH₂SO₂CH₃ | n-Pr | Ph-4-OCF₃ |
| H | C(CH₃)₂CH₂SO₂CH₃ | n-Pr | (L-45c)CF₃ |
| H | C(CH₃)₂CH₂SO₂CH₃ | i-Pr | Ph-4-Br |
| H | C(CH₃)₂CH₂SO₂CH₃ | i-Pr | Ph-4-CF₃ |
| H | C(CH₃)₂CH₂SO₂CH₃ | i-Pr | Ph-4-OCF₃ |
| H | C(CH₃)₂CH₂SO₂CH₃ | i-Pr | (L-45c)CF₃ |
| H | C(CH₃)₂CH₂SO₂CH₃ | c-Pr | Ph-4-Br |
| H | C(CH₃)₂CH₂SO₂CH₃ | c-Pr | Ph-4-CF₃ |
| H | C(CH₃)₂CH₂SO₂CH₃ | c-Pr | Ph-4-OCF₃ |
| H | C(CH₃)₂CH₂SO₂CH₃ | c-Pr | (L-45c)CF₃ |
| H | C(CH₃)₂CH₂SO₂CH₃ | n-Bu | Ph-4-OCF₃ |
| H | C(CH₃)₂CH₂SO₂CH₃ | CH₂F | (L-45c)CF₃ |
| H | C(CH₃)₂CH₂SO₂CH₃ | CH₂Cl | Ph-4-Br |
| H | C(CH₃)₂CH₂SO₂CH₃ | CH₂Br | Ph-4-CF₃ |
| H | C(CH₃)₂CH₂SO₂CH₃ | CHF₂ | Ph-4-Cl |
| H | C(CH₃)₂CH₂SO₂CH₃ | CHF₂ | Ph-4-Br |
| H | C(CH₃)₂CH₂SO₂CH₃ | CHF₂ | Ph-4-CF₃ |
| H | C(CH₃)₂CH₂SO₂CH₃ | CHF₂ | Ph-4-OCF₃ |
| H | C(CH₃)₂CH₂SO₂CH₃ | CHF₂ | Ph-4-OCF₂Br |
| H | C(CH₃)₂CH₂SO₂CH₃ | CHF₂ | Ph-4-OCF₂CHFCF₃ |
| H | C(CH₃)₂CH₂SO₂CH₃ | CHF₂ | Ph-4-OCF₂CHFOCF₃ |
| H | C(CH₃)₂CH₂SO₂CH₃ | CHF₂ | Ph-4-OCF₂CHFOCF₂CF₂CF₃ |
| H | C(CH₃)₂CH₂SO₂CH₃ | CHF₂ | Ph-4-O(L-45g) |
| H | C(CH₃)₂CH₂SO₂CH₃ | CHF₂ | Ph-3,4-Cl₂ |
| H | C(CH₃)₂CH₂SO₂CH₃ | CHF₂ | Ph(-3-OCF₂O-4-) |
| H | C(CH₃)₂CH₂SO₂CH₃ | CHF₂ | Ph(-3-OCF₂CF₂O-4-) |
| H | C(CH₃)₂CH₂SO₂CH₃ | CHF₂ | (L-45c)CF₃ |
| H | C(CH₃)₂CH₂SO₂CH₃ | CHF₂ | L-45f |
| H | C(CH₃)₂CH₂SO₂CH₃ | CHF₂ | (L-46c)CF₃ |
| H | C(CH₃)₂CH₂SO₂CH₃ | CHF₂ | L-46d |
| H | C(CH₃)₂CH₂SO₂CH₃ | CHFCl | Ph-4-OCF₃ |
| H | C(CH₃)₂CH₂SO₂CH₃ | CHFBr | (L-45c)CF₃ |
| H | C(CH₃)₂CH₂SO₂CH₃ | CF₃ | c-Pr |
| H | C(CH₃)₂CH₂SO₂CH₃ | CF₃ | c-Bu |
| H | C(CH₃)₂CH₂SO₂CH₃ | CF₃ | c-Pen |
| H | C(CH₃)₂CH₂SO₂CH₃ | CF₃ | c-Hex |
| H | C(CH₃)₂CH₂SO₂CH₃ | CF₃ | T-1 |
| H | C(CH₃)₂CH₂SO₂CH₃ | CF₃ | T-2 |
| H | C(CH₃)₂CH₂SO₂CH₃ | CF₃ | T-3 |
| H | C(CH₃)₂CH₂SO₂CH₃ | CF₃ | T-4 |
| H | C(CH₃)₂CH₂SO₂CH₃ | CF₃ | T-5 |
| H | C(CH₃)₂CH₂SO₂CH₃ | CF₃ | CH₂OCH₃ |
| H | C(CH₃)₂CH₂SO₂CH₃ | CF₃ | CH₂OEt |
| H | C(CH₃)₂CH₂SO₂CH₃ | CF₃ | CH₂OPr-n |
| H | C(CH₃)₂CH₂SO₂CH₃ | CF₃ | CH₂OPr-i |
| H | C(CH₃)₂CH₂SO₂CH₃ | CF₃ | CH₂OBu-n |
| H | C(CH₃)₂CH₂SO₂CH₃ | CF₃ | CH₂OC(O)(Ph-2-Cl) |
| H | C(CH₃)₂CH₂SO₂CH₃ | CF₃ | CH₂OC(O)(Ph-3-Cl) |
| H | C(CH₃)₂CH₂SO₂CH₃ | CF₃ | CH₂OC(O)(Ph-4-Cl) |
| H | C(CH₃)₂CH₂SO₂CH₃ | CF₃ | CH₂OPh |
| H | C(CH₃)₂CH₂SO₂CH₃ | CF₃ | CH₂O(Ph-2-F) |
| H | C(CH₃)₂CH₂SO₂CH₃ | CF₃ | CH₂O(Ph-3-F) |
| H | C(CH₃)₂CH₂SO₂CH₃ | CF₃ | CH₂O(Ph-4-F) |
| H | C(CH₃)₂CH₂SO₂CH₃ | CF₃ | CH₂O(Ph-2-Cl) |
| H | C(CH₃)₂CH₂SO₂CH₃ | CF₃ | CH₂O(Ph-3-Cl) |
| H | C(CH₃)₂CH₂SO₂CH₃ | CF₃ | CH₂O(Ph-4-Cl) |
| H | C(CH₃)₂CH₂SO₂CH₃ | CF₃ | CH₂O(Ph-2-Br) |
| H | C(CH₃)₂CH₂SO₂CH₃ | CF₃ | CH₂O(Ph-3-Br) |
| H | C(CH₃)₂CH₂SO₂CH₃ | CF₃ | CH₂O(Ph-4-Br) |
| H | C(CH₃)₂CH₂SO₂CH₃ | CF₃ | CH₂O(Ph-2-CF₃) |
| H | C(CH₃)₂CH₂SO₂CH₃ | CF₃ | CH₂O(Ph-3-CF₃) |
| H | C(CH₃)₂CH₂SO₂CH₃ | CF₃ | CH₂O(Ph-4-CF₃) |
| H | C(CH₃)₂CH₂SO₂CH₃ | CF₃ | CH₂O(Ph-2-OCF₃) |
| H | C(CH₃)₂CH₂SO₂CH₃ | CF₃ | CH₂O(Ph-3-OCF₃) |
| H | C(CH₃)₂CH₂SO₂CH₃ | CF₃ | CH₂O(Ph-4-OCF₃) |
| H | C(CH₃)₂CH₂SO₂CH₃ | CF₃ | CF₂OPh |
| H | C(CH₃)₂CH₂SO₂CH₃ | CF₃ | CF₂O(Ph-2-F) |
| H | C(CH₃)₂CH₂SO₂CH₃ | CF₃ | CF₂O(Ph-3-F) |
| H | C(CH₃)₂CH₂SO₂CH₃ | CF₃ | CF₂O(Ph-4-F) |
| H | C(CH₃)₂CH₂SO₂CH₃ | CF₃ | CF₂O(Ph-2-Cl) |
| H | C(CH₃)₂CH₂SO₂CH₃ | CF₃ | CF₂O(Ph-3-Cl) |
| H | C(CH₃)₂CH₂SO₂CH₃ | CF₃ | CF₂O(Ph-4-Cl) |
| H | C(CH₃)₂CH₂SO₂CH₃ | CF₃ | CF₂O(Ph-2-Br) |
| H | C(CH₃)₂CH₂SO₂CH₃ | CF₃ | CF₂O(Ph-3-Br) |
| H | C(CH₃)₂CH₂SO₂CH₃ | CF₃ | CF₂O(Ph-4-Br) |
| H | C(CH₃)₂CH₂SO₂CH₃ | CF₃ | CF₂O(Ph-2-CF₃) |
| H | C(CH₃)₂CH₂SO₂CH₃ | CF₃ | CF₂O(Ph-3-CF₃) |
| H | C(CH₃)₂CH₂SO₂CH₃ | CF₃ | CF₂O(Ph-4-CF₃) |
| H | C(CH₃)₂CH₂SO₂CH₃ | CF₃ | CF₂O(Ph-2-OCF₃) |
| H | C(CH₃)₂CH₂SO₂CH₃ | CF₃ | CF₂O(Ph-3-OCF₃) |
| H | C(CH₃)₂CH₂SO₂CH₃ | CF₃ | CF₂O(Ph-4-OCF₃) |
| H | C(CH₃)₂CH₂SO₂CH₃ | CF₃ | CH₂S(Ph-2-Cl) |
| H | C(CH₃)₂CH₂SO₂CH₃ | CF₃ | CH₂S(Ph-3-Cl) |
| H | C(CH₃)₂CH₂SO₂CH₃ | CF₃ | CH₂S(Ph-4-Cl) |
| H | C(CH₃)₂CH₂SO₂CH₃ | CF₃ | CH₂SO₂(Ph-2-Cl) |
| H | C(CH₃)₂CH₂SO₂CH₃ | CF₃ | CH₂SO₂(Ph-3-Cl) |
| H | C(CH₃)₂CH₂SO₂CH₃ | CF₃ | CH₂SO₂(Ph-4-Cl) |
| H | C(CH₃)₂CH₂SO₂CH₃ | CF₃ | CH₂N(CH₃)₂ |
| H | C(CH₃)₂CH₂SO₂CH₃ | CF₃ | CH₂NH(Ph-2-Cl) |
| H | C(CH₃)₂CH₂SO₂CH₃ | CF₃ | CH₂NH(Ph-3-Cl) |
| H | C(CH₃)₂CH₂SO₂CH₃ | CF₃ | CH₂NH(Ph-4-Cl) |
| H | C(CH₃)₂CH₂SO₂CH₃ | CF₃ | CF₂C(O)OEt |
| H | C(CH₃)₂CH₂SO₂CH₃ | CF₃ | CH₂(L-5a) |
| H | C(CH₃)₂CH₂SO₂CH₃ | CF₃ | CH₂(L-14a) |
| H | C(CH₃)₂CH₂SO₂CH₃ | CF₃ | CH₂(L-24a) |
| H | C(CH₃)₂CH₂SO₂CH₃ | CF₃ | CH₂(L-36a) |
| H | C(CH₃)₂CH₂SO₂CH₃ | CF₃ | CH₂CH₂Ph |
| H | C(CH₃)₂CH₂SO₂CH₃ | CF₃ | CH₂CH₂(Ph-2-Cl) |
| H | C(CH₃)₂CH₂SO₂CH₃ | CF₃ | CH₂CH₂(Ph-3-Cl) |
| H | C(CH₃)₂CH₂SO₂CH₃ | CF₃ | CH₂CH₂(Ph-4-Cl) |
| H | C(CH₃)₂CH₂SO₂CH₃ | CF₃ | T-22 |
| H | C(CH₃)₂CH₂SO₂CH₃ | CF₃ | T-23 |
| H | C(CH₃)₂CH₂SO₂CH₃ | CF₃ | T-24 |
| H | C(CH₃)₂CH₂SO₂CH₃ | CF₃ | C(O)OEt |
| H | C(CH₃)₂CH₂SO₂CH₃ | CF₃ | C(O)OBu-t |
| H | C(CH₃)₂CH₂SO₂CH₃ | CF₃ | C(O)OCH₂CF₃ |
| H | C(CH₃)₂CH₂SO₂CH₃ | CF₃ | CH═CH(Ph-2-F) |
| H | C(CH₃)₂CH₂SO₂CH₃ | CF₃ | CH═CH(Ph-3-F) |
| H | C(CH₃)₂CH₂SO₂CH₃ | CF₃ | CH═CH(Ph-4-F) |
| H | C(CH₃)₂CH₂SO₂CH₃ | CF₃ | CH═CH(Ph-2-Cl) |
| H | C(CH₃)₂CH₂SO₂CH₃ | CF₃ | CH═CH(Ph-3-Cl) |
| H | C(CH₃)₂CH₂SO₂CH₃ | CF₃ | CH═CH(Ph-4-Cl) |
| H | C(CH₃)₂CH₂SO₂CH₃ | CF₃ | CH═CH(Ph-2-Br) |
| H | C(CH₃)₂CH₂SO₂CH₃ | CF₃ | CH═CH(Ph-3-Br) |
| H | C(CH₃)₂CH₂SO₂CH₃ | CF₃ | CH═CH(Ph-4-Br) |
| H | C(CH₃)₂CH₂SO₂CH₃ | CF₃ | CH═CH(Ph-2-CF₃) |
| H | C(CH₃)₂CH₂SO₂CH₃ | CF₃ | CH═CH(Ph-3-CF₃) |
| H | C(CH₃)₂CH₂SO₂CH₃ | CF₃ | CH═CH(Ph-4-CF₃) |
| H | C(CH₃)₂CH₂SO₂CH₃ | CF₃ | CH═CH(Ph-2-OCF₃) |
| H | C(CH₃)₂CH₂SO₂CH₃ | CF₃ | CH═CH(Ph-3-OCF₃) |
| H | C(CH₃)₂CH₂SO₂CH₃ | CF₃ | CH═CH(Ph-4-OCF₃) |
| H | C(CH₃)₂CH₂SO₂CH₃ | CF₃ | CH═CH(Ph-2-SCH₃) |
| H | C(CH₃)₂CH₂SO₂CH₃ | CF₃ | CH═CH(Ph-3-SCH₃) |
| H | C(CH₃)₂CH₂SO₂CH₃ | CF₃ | CH═CH(Ph-4-SCH₃) |
| H | C(CH₃)₂CH₂SO₂CH₃ | CF₃ | CH═CH(Ph-2-SO₂CH₃) |
| H | C(CH₃)₂CH₂SO₂CH₃ | CF₃ | CH═CH(Ph-3-SO₂CH₃) |
| H | C(CH₃)₂CH₂SO₂CH₃ | CF₃ | CH═CH(Ph-4-SO₂CH₃) |
| H | C(CH₃)₂CH₂SO₂CH₃ | CF₃ | CH═CH(Ph-3,4-F₂) |
| H | C(CH₃)₂CH₂SO₂CH₃ | CF₃ | CH═CH(Ph-3-F-4-Cl) |
| H | C(CH₃)₂CH₂SO₂CH₃ | CF₃ | CH═CH(Ph-3,4-Cl₂) |
| H | C(CH₃)₂CH₂SO₂CH₃ | CF₃ | CH═CH(Ph-3,4-Br₂) |
| H | C(CH₃)₂CH₂SO₂CH₃ | CF₃ | CH═CH(Ph-3-F-4-CF₃) |
| H | C(CH₃)₂CH₂SO₂CH₃ | CF₃ | CH═CH(Ph-3-Cl-4-OCF₃) |
| H | C(CH₃)₂CH₂SO₂CH₃ | CF₃ | CH═CH[Ph(-3-OCF₂O-4-)] |
| H | C(CH₃)₂CH₂SO₂CH₃ | CF₃ | Ph |
| H | C(CH₃)₂CH₂SO₂CH₃ | CF₃ | Ph-2-F |
| H | C(CH₃)₂CH₂SO₂CH₃ | CF₃ | Ph-3-F |
| H | C(CH₃)₂CH₂SO₂CH₃ | CF₃ | Ph-4-F |
| H | C(CH₃)₂CH₂SO₂CH₃ | CF₃ | Ph-2-Cl |
| H | C(CH₃)₂CH₂SO₂CH₃ | CF₃ | Ph-3-Cl |
| H | C(CH₃)₂CH₂SO₂CH₃ | CF₃ | Ph-4-Cl |
| H | C(CH₃)₂CH₂SO₂CH₃ | CF₃ | Ph-2-Br |
| H | C(CH₃)₂CH₂SO₂CH₃ | CF₃ | Ph-3-Br |
| H | C(CH₃)₂CH₂SO₂CH₃ | CF₃ | Ph-4-Br |
| H | C(CH₃)₂CH₂SO₂CH₃ | CF₃ | Ph-4-I |
| H | C(CH₃)₂CH₂SO₂CH₃ | CF₃ | Ph-4-Bu-t |
| H | C(CH₃)₂CH₂SO₂CH₃ | CF₃ | Ph-2-CF₃ |
| H | C(CH₃)₂CH₂SO₂CH₃ | CF₃ | Ph-3-CF₃ |
| H | C(CH₃)₂CH₂SO₂CH₃ | CF₃ | Ph-4-CF₃ |
| H | C(CH₃)₂CH₂SO₂CH₃ | CF₃ | Ph-4-OCH₃ |
| H | C(CH₃)₂CH₂SO₂CH₃ | CF₃ | Ph-4-OCHF₂ |
| H | C(CH₃)₂CH₂SO₂CH₃ | CF₃ | Ph-2-OCF₃ |
| H | C(CH₃)₂CH₂SO₂CH₃ | CF₃ | Ph-3-OCF₃ |
| H | C(CH₃)₂CH₂SO₂CH₃ | CF₃ | Ph-4-OCF₃ |
| H | C(CH₃)₂CH₂SO₂CH₃ | CF₃ | Ph-4-OCF₂Br |
| H | C(CH₃)₂CH₂SO₂CH₃ | CF₃ | Ph-4-OCH₂CF₃ |
| H | C(CH₃)₂CH₂SO₂CH₃ | CF₃ | Ph-4-OCF₂CHF₂ |
| H | C(CH₃)₂CH₂SO₂CH₃ | CF₃ | Ph-4-OCF₂CHFCl |
| H | C(CH₃)₂CH₂SO₂CH₃ | CF₃ | Ph-4-OCF₂CHFBr |
| H | C(CH₃)₂CH₂SO₂CH₃ | CF₃ | Ph-4-OCF₂CF₂Br |
| H | C(CH₃)₂CH₂SO₂CH₃ | CF₃ | Ph-4-OCF₂CFCl₂ |
| H | C(CH₃)₂CH₂SO₂CH₃ | CF₃ | Ph-4-OCF₂CCl₃ |
| H | C(CH₃)₂CH₂SO₂CH₃ | CF₃ | Ph-4-OCH₂CF₂CHF₂ |
| H | C(CH₃)₂CH₂SO₂CH₃ | CF₃ | Ph-3-OCF₂CHFCF₃ |
| H | C(CH₃)₂CH₂SO₂CH₃ | CF₃ | Ph-4-OCF₂CHFCF₃ |
| H | C(CH₃)₂CH₂SO₂CH₃ | CF₃ | Ph-4-OCH(CF₃)₂ |
| H | C(CH₃)₂CH₂SO₂CH₃ | CF₃ | Ph-4-OCF₂CFBrCF₃ |
| H | C(CH₃)₂CH₂SO₂CH₃ | CF₃ | Ph-3-OCF₂CHFOCF₃ |
| H | C(CH₃)₂CH₂SO₂CH₃ | CF₃ | Ph-4-OCF₂CHFOCF₃ |
| H | C(CH₃)₂CH₂SO₂CH₃ | CF₃ | Ph-4-OCF₂CHFOCF₂CF₂CF₃ |
| H | C(CH₃)₂CH₂SO₂CH₃ | CF₃ | Ph-4-OSO₂CH₃ |
| H | C(CH₃)₂CH₂SO₂CH₃ | CF₃ | Ph-4-OSO₂CF₃ |
| H | C(CH₃)₂CH₂SO₂CH₃ | CF₃ | Ph-3-O(Ph-4-Cl) |
| H | C(CH₃)₂CH₂SO₂CH₃ | CF₃ | Ph-4-O(Ph-4-Cl) |
| H | C(CH₃)₂CH₂SO₂CH₃ | CF₃ | Ph-4-O(Ph-4-Br) |
| H | C(CH₃)₂CH₂SO₂CH₃ | CF₃ | Ph-4-O(Ph-4-CF₃) |
| H | C(CH₃)₂CH₂SO₂CH₃ | CF₃ | Ph-4-O(L-45c)Br |
| H | C(CH₃)₂CH₂SO₂CH₃ | CF₃ | Ph-4-O(L-45c)CF₃ |
| H | C(CH₃)₂CH₂SO₂CH₃ | CF₃ | Ph-3-O(L-45e) |
| H | C(CH₃)₂CH₂SO₂CH₃ | CF₃ | Ph-4-O(L-45e) |
| H | C(CH₃)₂CH₂SO₂CH₃ | CF₃ | Ph-4-O(L-48b)Br |
| H | C(CH₃)₂CH₂SO₂CH₃ | CF₃ | Ph-4-SCH₃ |
| H | C(CH₃)₂CH₂SO₂CH₃ | CF₃ | Ph-4-SO₂CH₃ |
| H | C(CH₃)₂CH₂SO₂CH₃ | CF₃ | Ph-3-S(Ph-4-Cl) |
| H | C(CH₃)₂CH₂SO₂CH₃ | CF₃ | Ph-4-S(Ph-4-Cl) |
| H | C(CH₃)₂CH₂SO₂CH₃ | CF₃ | Ph-4-S(Ph-4-Br) |
| H | C(CH₃)₂CH₂SO₂CH₃ | CF₃ | Ph-4-S(Ph-4-CF₃) |
| H | C(CH₃)₂CH₂SO₂CH₃ | CF₃ | Ph-4-S(L-45c)Br |
| H | C(CH₃)₂CH₂SO₂CH₃ | CF₃ | Ph-4-S(L-45c)CF₃ |
| H | C(CH₃)₂CH₂SO₂CH₃ | CF₃ | Ph-4-S(L-45e) |
| H | C(CH₃)₂CH₂SO₂CH₃ | CF₃ | Ph-4-S(L-48b)Br |
| H | C(CH₃)₂CH₂SO₂CH₃ | CF₃ | Ph-4-NO₂ |
| H | C(CH₃)₂CH₂SO₂CH₃ | CF₃ | Ph-4-CN |
| H | C(CH₃)₂CH₂SO₂CH₃ | CF₃ | Ph-2,3-F₂ |
| H | C(CH₃)₂CH₂SO₂CH₃ | CF₃ | Ph-2,4-F₂ |
| H | C(CH₃)₂CH₂SO₂CH₃ | CF₃ | Ph-3,4-F₂ |
| H | C(CH₃)₂CH₂SO₂CH₃ | CF₃ | Ph-2,5-F₂ |
| H | C(CH₃)₂CH₂SO₂CH₃ | CF₃ | Ph-3,5-F₂ |
| H | C(CH₃)₂CH₂SO₂CH₃ | CF₃ | Ph-2-Cl-4-F |
| H | C(CH₃)₂CH₂SO₂CH₃ | CF₃ | Ph-2-F-3-Cl |
| H | C(CH₃)₂CH₂SO₂CH₃ | CF₃ | Ph-3-Cl-4-F |
| H | C(CH₃)₂CH₂SO₂CH₃ | CF₃ | Ph-2-F-4-Cl |
| H | C(CH₃)₂CH₂SO₂CH₃ | CF₃ | Ph-3-F-4-Cl |
| H | C(CH₃)₂CH₂SO₂CH₃ | CF₃ | Ph-2,3-Cl₂ |
| H | C(CH₃)₂CH₂SO₂CH₃ | CF₃ | Ph-2,4-Cl₂ |
| H | C(CH₃)₂CH₂SO₂CH₃ | CF₃ | Ph-2,5-Cl₂ |
| H | C(CH₃)₂CH₂SO₂CH₃ | CF₃ | Ph-3,4-Cl₂ |
| H | C(CH₃)₂CH₂SO₂CH₃ | CF₃ | Ph-3,5-Cl₂ |
| H | C(CH₃)₂CH₂SO₂CH₃ | CF₃ | Ph-3-Br-4-F |
| H | C(CH₃)₂CH₂SO₂CH₃ | CF₃ | Ph-2-F-4-Br |
| H | C(CH₃)₂CH₂SO₂CH₃ | CF₃ | Ph-2-F-5-Br |
| H | C(CH₃)₂CH₂SO₂CH₃ | CF₃ | Ph-3,4-Br₂ |
| H | C(CH₃)₂CH₂SO₂CH₃ | CF₃ | Ph-3,5-Br₂ |
| H | C(CH₃)₂CH₂SO₂CH₃ | CF₃ | Ph-3-CH₃-4-F |
| H | C(CH₃)₂CH₂SO₂CH₃ | CF₃ | Ph-3-F-4-CH₃ |
| H | C(CH₃)₂CH₂SO₂CH₃ | CF₃ | Ph-2-F-5-CH₃ |
| H | C(CH₃)₂CH₂SO₂CH₃ | CF₃ | Ph-2,4-(CH₃)₂ |
| H | C(CH₃)₂CH₂SO₂CH₃ | CF₃ | Ph-3,4-(CH₃)₂ |
| H | C(CH₃)₂CH₂SO₂CH₃ | CF₃ | Ph-2-F-3-CF₃ |
| H | C(CH₃)₂CH₂SO₂CH₃ | CF₃ | Ph-3-CF₃-4-F |
| H | C(CH₃)₂CH₂SO₂CH₃ | CF₃ | Ph-3-CF₃-4-Cl |
| H | C(CH₃)₂CH₂SO₂CH₃ | CF₃ | Ph-2-F-4-CF₃ |
| H | C(CH₃)₂CH₂SO₂CH₃ | CF₃ | Ph-3-F-4-CF₃ |
| H | C(CH₃)₂CH₂SO₂CH₃ | CF₃ | Ph-2-F-5-CF₃ |
| H | C(CH₃)₂CH₂SO₂CH₃ | CF₃ | Ph-3-F-5-CF₃ |
| H | C(CH₃)₂CH₂SO₂CH₃ | CF₃ | Ph-2-Cl-4-CF₃ |
| H | C(CH₃)₂CH₂SO₂CH₃ | CF₃ | Ph-3,5-(CF₃)₂ |
| H | C(CH₃)₂CH₂SO₂CH₃ | CF₃ | Ph-3-Br-4-OCH₃ |
| H | C(CH₃)₂CH₂SO₂CH₃ | CF₃ | Ph-3-F-4-OCHF₂ |
| H | C(CH₃)₂CH₂SO₂CH₃ | CF₃ | Ph-3-Cl-4-OCHF₂ |
| H | C(CH₃)₂CH₂SO₂CH₃ | CF₃ | Ph-3-Br-4-OCHF₂ |
| H | C(CH₃)₂CH₂SO₂CH₃ | CF₃ | Ph-3-F-4-OCF₃ |
| H | C(CH₃)₂CH₂SO₂CH₃ | CF₃ | Ph-3-Cl-4-OCF₃ |
| H | C(CH₃)₂CH₂SO₂CH₃ | CF₃ | Ph-3-Br-4-OCF₃ |
| H | C(CH₃)₂CH₂SO₂CH₃ | CF₃ | Ph-3-F-4-OCF₂Br |
| H | C(CH₃)₂CH₂SO₂CH₃ | CF₃ | Ph-3-Cl-4-OCF₂Br |
| H | C(CH₃)₂CH₂SO₂CH₃ | CF₃ | Ph-3-Br-4-OCF₂Br |
| H | C(CH₃)₂CH₂SO₂CH₃ | CF₃ | Ph-3-F-4-OCF₂CHF₂ |
| H | C(CH₃)₂CH₂SO₂CH₃ | CF₃ | Ph-3-Cl-4-OCF₂CHF₂ |
| H | C(CH₃)₂CH₂SO₂CH₃ | CF₃ | Ph-3-Br-4-OCF₂CHF₂ |
| H | C(CH₃)₂CH₂SO₂CH₃ | CF₃ | Ph-3-F-4-OCF₂CHFCl |
| H | C(CH₃)₂CH₂SO₂CH₃ | CF₃ | Ph-3-Cl-4-OCF₂CHFCl |
| H | C(CH₃)₂CH₂SO₂CH₃ | CF₃ | Ph-3-Br-4-OCF₂CHFCl |
| H | C(CH₃)₂CH₂SO₂CH₃ | CF₃ | Ph-3-F-4-OCF₂CHFCF₃ |
| H | C(CH₃)₂CH₂SO₂CH₃ | CF₃ | Ph-3-Cl-4-OCF₂CHFCF₃ |
| H | C(CH₃)₂CH₂SO₂CH₃ | CF₃ | Ph-3-Br-4-OCF₂CHFCF₃ |
| H | C(CH₃)₂CH₂SO₂CH₃ | CF₃ | Ph-3-F-4-OCF₂CHFOCF₃ |
| H | C(CH₃)₂CH₂SO₂CH₃ | CF₃ | Ph-3-Cl-4-OCF₂CHFOCF₃ |
| H | C(CH₃)₂CH₂SO₂CH₃ | CF₃ | Ph-3-Br-4-OCF₂CHFOCF₃ |
| H | C(CH₃)₂CH₂SO₂CH₃ | CF₃ | Ph-3-CH₃-4-OCF₂CHFOCF₃ |
| H | C(CH₃)₂CH₂SO₂CH₃ | CF₃ | Ph-3-F-4-OCF₂CHFOCF₂CF₂CF₃ |
| H | C(CH₃)₂CH₂SO₂CH₃ | CF₃ | Ph-3-Cl-4-OCF₂CHFOCF₂CF₂CF₃ |
| H | C(CH₃)₂CH₂SO₂CH₃ | CF₃ | Ph-3-Br-4-OCF₂CHFOCF₂CF₂CF₃ |
| H | C(CH₃)₂CH₂SO₂CH₃ | CF₃ | Ph(-3-OCF₂O-4-) |
| H | C(CH₃)₂CH₂SO₂CH₃ | CF₃ | Ph(-3-OCF₂CF₂O-4-) |
| H | C(CH₃)₂CH₂SO₂CH₃ | CF₃ | Ph-3-OPh-4-F |
| H | C(CH₃)₂CH₂SO₂CH₃ | CF₃ | Ph-3-NO₂-4-F |
| H | C(CH₃)₂CH₂SO₂CH₃ | CF₃ | Ph-3-NO₂-4-Cl |
| H | C(CH₃)₂CH₂SO₂CH₃ | CF₃ | Ph-2-F-5-NO₂ |
| H | C(CH₃)₂CH₂SO₂CH₃ | CF₃ | Ph-3-CN-4-F |
| H | C(CH₃)₂CH₂SO₂CH₃ | CF₃ | Ph-2,3,4-F₃ |
| H | C(CH₃)₂CH₂SO₂CH₃ | CF₃ | Ph-2,3,5-F₃ |
| H | C(CH₃)₂CH₂SO₂CH₃ | CF₃ | Ph-2,4,5-F₃ |
| H | C(CH₃)₂CH₂SO₂CH₃ | CF₃ | Ph-3,4,5-F₃ |
| H | C(CH₃)₂CH₂SO₂CH₃ | CF₃ | Ph-2,3-F₂-4-CH₃ |
| H | C(CH₃)₂CH₂SO₂CH₃ | CF₃ | Ph-2,3-F₂-4-CF₃ |
| H | C(CH₃)₂CH₂SO₂CH₃ | CF₃ | Ph-3,4-F₂-5-CF₃ |
| H | C(CH₃)₂CH₂SO₂CH₃ | CF₃ | Ph-2-F-3-Cl-5-CF₃ |
| H | C(CH₃)₂CH₂SO₂CH₃ | CF₃ | Ph-3,5-Cl₂-4-OCH₃ |
| H | C(CH₃)₂CH₂SO₂CH₃ | CF₃ | 1-Naph |
| H | C(CH₃)₂CH₂SO₂CH₃ | CF₃ | 2-Naph |
| H | C(CH₃)₂CH₂SO₂CH₃ | CF₃ | (L-1b)Br |
| H | C(CH₃)₂CH₂SO₂CH₃ | CF₃ | (L-1c)Cl |
| H | C(CH₃)₂CH₂SO₂CH₃ | CF₃ | (L-1c)Br |
| H | C(CH₃)₂CH₂SO₂CH₃ | CF₃ | (L-1c)I |
| H | C(CH₃)₂CH₂SO₂CH₃ | CF₃ | (L-1c)CF₃ |
| H | C(CH₃)₂CH₂SO₂CH₃ | CF₃ | (L-2b)Br |
| H | C(CH₃)₂CH₂SO₂CH₃ | CF₃ | (L-3b)Cl |
| H | C(CH₃)₂CH₂SO₂CH₃ | CF₃ | (L-3b)Br |
| H | C(CH₃)₂CH₂SO₂CH₃ | CF₃ | (L-3c)F |
| H | C(CH₃)₂CH₂SO₂CH₃ | CF₃ | (L-3c)Cl |
| H | C(CH₃)₂CH₂SO₂CH₃ | CF₃ | (L-3c)Br |
| H | C(CH₃)₂CH₂SO₂CH₃ | CF₃ | (L-3c)I |
| H | C(CH₃)₂CH₂SO₂CH₃ | CF₃ | (L-3c)CF₃ |
| H | C(CH₃)₂CH₂SO₂CH₃ | CF₃ | (L-3c)CN |
| H | C(CH₃)₂CH₂SO₂CH₃ | CF₃ | L-3d |
| H | C(CH₃)₂CH₂SO₂CH₃ | CF₃ | (L-4b)Cl |
| H | C(CH₃)₂CH₂SO₂CH₃ | CF₃ | (L-4b)Br |
| H | C(CH₃)₂CH₂SO₂CH₃ | CF₃ | (L-4b)CN |
| H | C(CH₃)₂CH₂SO₂CH₃ | CF₃ | (L-10b)Cl |
| H | C(CH₃)₂CH₂SO₂CH₃ | CF₃ | (L-10b)Br |
| H | C(CH₃)₂CH₂SO₂CH₃ | CF₃ | (L-15b)CF₃ |
| H | C(CH₃)₂CH₂SO₂CH₃ | CF₃ | (L-16a)CHF₂ |
| H | C(CH₃)₂CH₂SO₂CH₃ | CF₃ | (L-16a)CF₂Br |
| H | C(CH₃)₂CH₂SO₂CH₃ | CF₃ | (L-17a)Cl |
| H | C(CH₃)₂CH₂SO₂CH₃ | CF₃ | (L-21b)Cl |
| H | C(CH₃)₂CH₂SO₂CH₃ | CF₃ | (L-21b)Br |
| H | C(CH₃)₂CH₂SO₂CH₃ | CF₃ | (L-21b)I |
| H | C(CH₃)₂CH₂SO₂CH₃ | CF₃ | (L-21b)CF₃ |
| H | C(CH₃)₂CH₂SO₂CH₃ | CF₃ | (L-22b)Cl |
| H | C(CH₃)₂CH₂SO₂CH₃ | CF₃ | (L-22b)Br |
| H | C(CH₃)₂CH₂SO₂CH₃ | CF₃ | (L-23b)Cl |
| H | C(CH₃)₂CH₂SO₂CH₃ | CF₃ | (L-23b)Br |
| H | C(CH₃)₂CH₂SO₂CH₃ | CF₃ | (L-23c)Cl |
| H | C(CH₃)₂CH₂SO₂CH₃ | CF₃ | (L-23c)Br |
| H | C(CH₃)₂CH₂SO₂CH₃ | CF₃ | (L-31a)Cl |
| H | C(CH₃)₂CH₂SO₂CH₃ | CF₃ | (L-31a)Br |
| H | C(CH₃)₂CH₂SO₂CH₃ | CF₃ | (L-45c)F |
| H | C(CH₃)₂CH₂SO₂CH₃ | CF₃ | (L-45c)Cl |
| H | C(CH₃)₂CH₂SO₂CH₃ | CF₃ | (L-45c)Br |
| H | C(CH₃)₂CH₂SO₂CH₃ | CF₃ | (L-45c)I |
| H | C(CH₃)₂CH₂SO₂CH₃ | CF₃ | (L-45c)CF₃ |
| H | C(CH₃)₂CH₂SO₂CH₃ | CF₃ | L-45e |
| H | C(CH₃)₂CH₂SO₂CH₃ | CF₃ | L-45f |
| H | C(CH₃)₂CH₂SO₂CH₃ | CF₃ | (L-46c)F |
| H | C(CH₃)₂CH₂SO₂CH₃ | CF₃ | (L-46c)Cl |
| H | C(CH₃)₂CH₂SO₂CH₃ | CF₃ | (L-46c)Br |
| H | C(CH₃)₂CH₂SO₂CH₃ | CF₃ | (L-46c)I |
| H | C(CH₃)₂CH₂SO₂CH₃ | CF₃ | (L-46c)CF₃ |
| H | C(CH₃)₂CH₂SO₂CH₃ | CF₃ | (L-46c)OCH₂CF₃ |
| H | C(CH₃)₂CH₂SO₂CH₃ | CF₃ | (L-46c)OCH(CF₃)₂ |
| H | C(CH₃)₂CH₂SO₂CH₃ | CF₃ | L-46d |
| H | C(CH₃)₂CH₂SO₂CH₃ | CF₃ | L-47a |
| H | C(CH₃)₂CH₂SO₂CH₃ | CF₃ | L-47d |
| H | C(CH₃)₂CH₂SO₂CH₃ | CF₃ | (L-48b)Br |
| H | C(CH₃)₂CH₂SO₂CH₃ | CF₃ | (L-50b)Cl |
| H | C(CH₃)₂CH₂SO₂CH₃ | CF₃ | (L-50b)Br |
| H | C(CH₃)₂CH₂SO₂CH₃ | CF₃ | (L-51b)Cl |
| H | C(CH₃)₂CH₂SO₂CH₃ | CF₂Cl | Ph-4-Cl |
| H | C(CH₃)₂CH₂SO₂CH₃ | CF₂Cl | Ph-4-Br |
| H | C(CH₃)₂CH₂SO₂CH₃ | CF₂Cl | Ph-4-CF₃ |
| H | C(CH₃)₂CH₂SO₂CH₃ | CF₂Cl | Ph-4-OCF₃ |
| H | C(CH₃)₂CH₂SO₂CH₃ | CF₂Cl | Ph-4-OCF₂Br |
| H | C(CH₃)₂CH₂SO₂CH₃ | CF₂Cl | Ph-4-OCF₂CHFCF₃ |
| H | C(CH₃)₂CH₂SO₂CH₃ | CF₂Cl | Ph-4-OCF₂CHFOCF₃ |
| H | C(CH₃)₂CH₂SO₂CH₃ | CF₂Cl | Ph-4-OCF₂CHFOCF₂CF₂CF₃ |
| H | C(CH₃)₂CH₂SO₂CH₃ | CF₂Cl | Ph-4-O(L-45g) |
| H | C(CH₃)₂CH₂SO₂CH₃ | CF₂Cl | Ph-3,4-Cl₂ |
| H | C(CH₃)₂CH₂SO₂CH₃ | CF₂Cl | Ph(-3-OCF₂O-4-) |
| H | C(CH₃)₂CH₂SO₂CH₃ | CF₂Cl | Ph(-3-OCF₂CF₂O-4-) |
| H | C(CH₃)₂CH₂SO₂CH₃ | CF₂Cl | (L-45c)CF₃ |
| H | C(CH₃)₂CH₂SO₂CH₃ | CF₂Cl | L-45f |
| H | C(CH₃)₂CH₂SO₂CH₃ | CF₂Cl | (L-46c)CF₃ |
| H | C(CH₃)₂CH₂SO₂CH₃ | CF₂Cl | L-46d |
| H | C(CH₃)₂CH₂SO₂CH₃ | CFCl₂ | Ph-4-Br |
| H | C(CH₃)₂CH₂SO₂CH₃ | CF₂Br | Ph-4-Cl |
| H | C(CH₃)₂CH₂SO₂CH₃ | CF₂Br | Ph-4-Br |
| H | C(CH₃)₂CH₂SO₂CH₃ | CF₂Br | Ph-4-CF₃ |
| H | C(CH₃)₂CH₂SO₂CH₃ | CF₂Br | Ph-4-OCF₃ |
| H | C(CH₃)₂CH₂SO₂CH₃ | CF₂Br | Ph-4-OCF₂Br |
| H | C(CH₃)₂CH₂SO₂CH₃ | CF₂Br | Ph-4-OCF₂CHFCF₃ |
| H | C(CH₃)₂CH₂SO₂CH₃ | CF₂Br | Ph-4-OCF₂CHFOCF₃ |
| H | C(CH₃)₂CH₂SO₂CH₃ | CF₂Br | Ph-4-OCF₂CHFOCF₂CF₂CF₃ |
| H | C(CH₃)₂CH₂SO₂CH₃ | CF₂Br | Ph-4-O(L-45g) |
| H | C(CH₃)₂CH₂SO₂CH₃ | CF₂Br | Ph-3,4-Cl₂ |
| H | C(CH₃)₂CH₂SO₂CH₃ | CF₂Br | Ph(-3-OCF₂O-4-) |
| H | C(CH₃)₂CH₂SO₂CH₃ | CF₂Br | Ph(-3-OCF₂CF₂O-4-) |
| H | C(CH₃)₂CH₂SO₂CH₃ | CF₂Br | (L-45c)CF₃ |
| H | C(CH₃)₂CH₂SO₂CH₃ | CF₂Br | L-45f |
| H | C(CH₃)₂CH₂SO₂CH₃ | CF₂Br | (L-46c)CF₃ |
| H | C(CH₃)₂CH₂SO₂CH₃ | CF₂Br | L-46d |
| H | C(CH₃)₂CH₂SO₂CH₃ | CFClBr | Ph-4-CF₃ |
| H | C(CH₃)₂CH₂SO₂CH₃ | CFBr₂ | Ph-4-OCF₃ |
| H | C(CH₃)₂CH₂SO₂CH₃ | CF₂CHF₂ | (L-45c)CF₃ |
| H | C(CH₃)₂CH₂SO₂CH₃ | CF₂CF₃ | Ph-4-Cl |
| H | C(CH₃)₂CH₂SO₂CH₃ | CF₂CF₃ | Ph-4-Br |
| H | C(CH₃)₂CH₂SO₂CH₃ | CF₂CF₃ | Ph-4-CF₃ |
| H | C(CH₃)₂CH₂SO₂CH₃ | CF₂CF₃ | Ph-4-OCF₃ |
| H | C(CH₃)₂CH₂SO₂CH₃ | CF₂CF₃ | Ph-4-OCF₂Br |
| H | C(CH₃)₂CH₂SO₂CH₃ | CF₂CF₃ | Ph-4-OCF₂CHFCF₃ |
| H | C(CH₃)₂CH₂SO₂CH₃ | CF₂CF₃ | Ph-4-OCF₂CHFOCF₃ |
| H | C(CH₃)₂CH₂SO₂CH₃ | CF₂CF₃ | Ph-4-OCF₂CHFOCF₂CF₂CF₃ |
| H | C(CH₃)₂CH₂SO₂CH₃ | CF₂CF₃ | Ph-4-O(L-45g) |
| H | C(CH₃)₂CH₂SO₂CH₃ | CF₂CF₃ | Ph-3,4-Cl₂ |
| H | C(CH₃)₂CH₂SO₂CH₃ | CF₂CF₃ | Ph(-3-OCF₂O-4-) |
| H | C(CH₃)₂CH₂SO₂CH₃ | CF₂CF₃ | Ph(-3-OCF₂CF₂O-4-) |
| H | C(CH₃)₂CH₂SO₂CH₃ | CF₂CF₃ | (L-45c)CF₃ |
| H | C(CH₃)₂CH₂SO₂CH₃ | CF₂CF₃ | L-45f |
| H | C(CH₃)₂CH₂SO₂CH₃ | CF₂CF₃ | (L-46c)CF₃ |
| H | C(CH₃)₂CH₂SO₂CH₃ | CF₂CF₃ | L-46d |
| H | C(CH₃)₂CH₂SO₂CH₃ | CF₂CF₂Cl | Ph-4-Br |
| H | C(CH₃)₂CH₂SO₂CH₃ | CFClCF₃ | Ph-4-CF₃ |
| H | C(CH₃)₂CH₂SO₂CH₃ | CFClCF₂Cl | Ph-4-OCF₃ |
| H | C(CH₃)₂CH₂SO₂CH₃ | CF₂CF₂Br | (L-45c)CF₃ |
| H | C(CH₃)₂CH₂SO₂CH₃ | CFBrCF₃ | Ph-4-Br |
| H | C(CH₃)₂CH₂SO₂CH₃ | CF₂CHFCF₃ | Ph-4-CF₃ |
| H | C(CH₃)₂CH₂SO₂CH₃ | CF₂CF₂CF₃ | Ph-4-Cl |
| H | C(CH₃)₂CH₂SO₂CH₃ | CF₂CF₂CF₃ | Ph-4-Br |
| H | C(CH₃)₂CH₂SO₂CH₃ | CF₂CF₂CF₃ | Ph-4-CF₃ |
| H | C(CH₃)₂CH₂SO₂CH₃ | CF₂CF₂CF₃ | Ph-4-OCF₃ |
| H | C(CH₃)₂CH₂SO₂CH₃ | CF₂CF₂CF₃ | Ph-4-OCF₂Br |
| H | C(CH₃)₂CH₂SO₂CH₃ | CF₂CF₂CF₃ | Ph-4-OCF₂CHFCF₃ |
| H | C(CH₃)₂CH₂SO₂CH₃ | CF₂CF₂CF₃ | Ph-4-OCF₂CHFOCF₃ |
| H | C(CH₃)₂CH₂SO₂CH₃ | CF₂CF₂CF₃ | Ph-4-OCF₂CHFOCF₂CF₂CF₃ |
| H | C(CH₃)₂CH₂SO₂CH₃ | CF₂CF₂CF₃ | Ph-4-O(L-45g) |
| H | C(CH₃)₂CH₂SO₂CH₃ | CF₂CF₂CF₃ | Ph-3,4-Cl₂ |
| H | C(CH₃)₂CH₂SO₂CH₃ | CF₂CF₂CF₃ | Ph(-3-OCF₂O-4-) |
| H | C(CH₃)₂CH₂SO₂CH₃ | CF₂CF₂CF₃ | Ph(-3-OCF₂CF₂O-4-) |
| H | C(CH₃)₂CH₂SO₂CH₃ | CF₂CF₂CF₃ | (L-45c)CF₃ |
| H | C(CH₃)₂CH₂SO₂CH₃ | CF₂CF₂CF₃ | L-45f |
| H | C(CH₃)₂CH₂SO₂CH₃ | CF₂CF₂CF₃ | (L-46c)CF₃ |
| H | C(CH₃)₂CH₂SO₂CH₃ | CF₂CF₂CF₃ | L-46d |
| H | C(CH₃)₂CH₂SO₂CH₃ | CF(CF₃)₂ | Ph-4-OCF₃ |
| H | C(CH₃)₂CH₂SO₂CH₃ | CF₂CF₂CF₂CF₃ | (L-45c)CF₃ |
| H | C(CH₃)₂CH₂SO₂CH₃ | T-1 | Ph-4-Br |
| H | C(CH₃)₂CH₂SO₂CH₃ | T-2 | Ph-4-CF₃ |
| H | C(CH₃)₂CH₂SO₂CH₃ | CH₂OCH₃ | Ph-4-OCF₃ |
| H | C(CH₃)₂CH₂SO₂CH₃ | CH₂OEt | (L-45c)CF₃ |
| H | C(CH₃)₂CH₂SO₂CH₃ | CH₂OCH₂CF₃ | Ph-4-Br |
| H | C(CH₃)₂CH₂SO₂CH₃ | CH₂OCH(CF₃)₂ | Ph-4-CF₃ |
| H | C(CH₃)₂CH₂SO₂CH₃ | CF₂OCH₃ | Ph-4-Cl |
| H | C(CH₃)₂CH₂SO₂CH₃ | CF₂OCH₃ | Ph-4-Br |
| H | C(CH₃)₂CH₂SO₂CH₃ | CF₂OCH₃ | Ph-4-CF₃ |
| H | C(CH₃)₂CH₂SO₂CH₃ | CF₂OCH₃ | Ph-4-OCF₃ |
| H | C(CH₃)₂CH₂SO₂CH₃ | CF₂OCH₃ | Ph-4-OCF₂Br |
| H | C(CH₃)₂CH₂SO₂CH₃ | CF₂OCH₃ | Ph-4-OCF₂CHFCF₃ |
| H | C(CH₃)₂CH₂SO₂CH₃ | CF₂OCH₃ | Ph-4-OCF₂CHFOCF₃ |
| H | C(CH₃)₂CH₂SO₂CH₃ | CF₂OCH₃ | Ph-4-OCF₂CHFOCF₂CF₂CF₃ |
| H | C(CH₃)₂CH₂SO₂CH₃ | CF₂OCH₃ | Ph-4-O(L-45g) |
| H | C(CH₃)₂CH₂SO₂CH₃ | CF₂OCH₃ | Ph-3,4-Cl₂ |
| H | C(CH₃)₂CH₂SO₂CH₃ | CF₂OCH₃ | Ph(-3-OCF₂O-4-) |
| H | C(CH₃)₂CH₂SO₂CH₃ | CF₂OCH₃ | Ph(-3-OCF₂CF₂O-4-) |
| H | C(CH₃)₂CH₂SO₂CH₃ | CF₂OCH₃ | (L-45c)CF₃ |
| H | C(CH₃)₂CH₂SO₂CH₃ | CF₂OCH₃ | L-45f |
| H | C(CH₃)₂CH₂SO₂CH₃ | CF₂OCH₃ | (L-46c)CF₃ |
| H | C(CH₃)₂CH₂SO₂CH₃ | CF₂OCH₃ | L-46d |
| H | C(CH₃)₂CH₂SO₂CH₃ | CH₂SCH₃ | Ph-4-OCF₃ |
| H | C(CH₃)₂CH₂SO₂CH₃ | CH₂SO₂CH₃ | (L-45c)CF₃ |
| H | C(CH₃)₂CH₂SO₂CH₃ | CH₂SCF₃ | Ph-4-Br |
| H | C(CH₃)₂CH₂SO₂CH₃ | CF₂SCH₃ | Ph-4-Cl |
| H | C(CH₃)₂CH₂SO₂CH₃ | CF₂SCH₃ | Ph-4-Br |
| H | C(CH₃)₂CH₂SO₂CH₃ | CF₂SCH₃ | Ph-4-CF₃ |
| H | C(CH₃)₂CH₂SO₂CH₃ | CF₂SCH₃ | Ph-4-OCF₃ |
| H | C(CH₃)₂CH₂SO₂CH₃ | CF₂SCH₃ | Ph-4-OCF₂Br |
| H | C(CH₃)₂CH₂SO₂CH₃ | CF₂SCH₃ | Ph-4-OCF₂CHFCF₃ |
| H | C(CH₃)₂CH₂SO₂CH₃ | CF₂SCH₃ | Ph-4-OCF₂CHFOCF₃ |
| H | C(CH₃)₂CH₂SO₂CH₃ | CF₂SCH₃ | Ph-4-OCF₂CHFOCF₂CF₂CF₃ |
| H | C(CH₃)₂CH₂SO₂CH₃ | CF₂SCH₃ | Ph-4-O(L-45g) |
| H | C(CH₃)₂CH₂SO₂CH₃ | CF₂SCH₃ | Ph-3,4-Cl₂ |
| H | C(CH₃)₂CH₂SO₂CH₃ | CF₂SCH₃ | Ph(-3-OCF₂O-4-) |
| H | C(CH₃)₂CH₂SO₂CH₃ | CF₂SCH₃ | Ph(-3-OCF₂CF₂O-4-) |
| H | C(CH₃)₂CH₂SO₂CH₃ | CF₂SCH₃ | (L-45c)CF₃ |
| H | C(CH₃)₂CH₂SO₂CH₃ | CF₂SCH₃ | L-45f |
| H | C(CH₃)₂CH₂SO₂CH₃ | CF₂SCH₃ | (L-46c)CF₃ |
| H | C(CH₃)₂CH₂SO₂CH₃ | CF₂SCH₃ | L-46d |
| H | C(CH₃)₂CH₂SO₂CH₃ | CF₂SEt | Ph-4-CF₃ |
| H | C(CH₃)₂CH₂SO₂CH₃ | CF₂SPr-n | Ph-4-OCF₃ |
| H | C(CH₃)₂CH₂SO₂CH₃ | CF₂SPr-i | (L-45c)CF₃ |
| H | C(CH₃)₂CH₂SO₂CH₃ | CF₂SCH₂Ph | Ph-4-Br |
| H | C(CH₃)₂CH₂SO₂CH₃ | CF₂SPh | Ph-4-CF₃ |
| H | C(CH₃)₂CH₂SO₂CH₃ | CF₂C(O)OEt | Ph-4-OCF₃ |
| H | C(CH₃)₂CH₂SO₂CH₃ | CF₂SO₂N(CH₃)₂ | (L-45c)CF₃ |
| H | C(CH₃)₂CH₂SO₂CH₃ | C(O)OEt | Ph-4-Br |
| H | C(CH₃)₂CH₂SO₂CH₃ | C(O)SEt | Ph-4-CF₃ |
| H | C(CH₃)₂CH₂SEt | CF₃ | CH═CH(Ph-4-Cl) |
| H | C(CH₃)₂CH₂SEt | CF₃ | Ph-4-Cl |
| H | C(CH₃)₂CH₂SEt | CF₃ | Ph-4-Br |
| H | C(CH₃)₂CH₂SEt | CF₃ | Ph-4-CF₃ |
| H | C(CH₃)₂CH₂SEt | CF₃ | Ph-4-OCF₃ |
| H | C(CH₃)₂CH₂SEt | CF₃ | Ph-4-OCF₂Br |
| H | C(CH₃)₂CH₂SEt | CF₃ | Ph-4-OCF₂CHFCF₃ |
| H | C(CH₃)₂CH₂SEt | CF₃ | Ph-4-OCF₂CHFOCF₃ |
| H | C(CH₃)₂CH₂SEt | CF₃ | Ph-4-OCF₂CHFOCF₂CF₂CF₃ |
| H | C(CH₃)₂CH₂SEt | CF₃ | Ph-4-O(L-45g) |
| H | C(CH₃)₂CH₂SEt | CF₃ | Ph-3,4-Cl₂ |
| H | C(CH₃)₂CH₂SEt | CF₃ | Ph(-3-OCF₂O-4-) |
| H | C(CH₃)₂CH₂SEt | CF₃ | Ph(-3-OCF₂CF₂O-4-) |
| H | C(CH₃)₂CH₂SEt | CF₃ | (L-45c)CF₃ |
| H | C(CH₃)₂CH₂SEt | CF₃ | L-45f |
| H | C(CH₃)₂CH₂SEt | CF₃ | (L-46c)CF₃ |
| H | C(CH₃)₂CH₂SEt | CF₃ | L-46d |
| H | C(CH₃)₂CH₂S(O)Et | CF₃ | CH═CH(Ph-4-Cl) |
| H | C(CH₃)₂CH₂S(O)Et | CF₃ | Ph-4-Cl |
| H | C(CH₃)₂CH₂S(O)Et | CF₃ | Ph-4-Br |
| H | C(CH₃)₂CH₂S(O)Et | CF₃ | Ph-4-CF₃ |
| H | C(CH₃)₂CH₂S(O)Et | CF₃ | Ph-4-OCF₃ |
| H | C(CH₃)₂CH₂S(O)Et | CF₃ | Ph-4-OCF₂Br |
| H | C(CH₃)₂CH₂S(O)Et | CF₃ | Ph-4-OCF₂CHFCF₃ |
| H | C(CH₃)₂CH₂S(O)Et | CF₃ | Ph-4-OCF₂CHFOCF₃ |
| H | C(CH₃)₂CH₂S(O)Et | CF₃ | Ph-4-OCF₂CHFOCF₂CF₂CF₃ |
| H | C(CH₃)₂CH₂S(O)Et | CF₃ | Ph-4-O(L-45g) |
| H | C(CH₃)₂CH₂S(O)Et | CF₃ | Ph-3,4-Cl₂ |
| H | C(CH₃)₂CH₂S(O)Et | CF₃ | Ph(-3-OCF₂O-4-) |
| H | C(CH₃)₂CH₂S(O)Et | CF₃ | Ph(-3-OCF₂CF₂O-4-) |
| H | C(CH₃)₂CH₂S(O)Et | CF₃ | (L-45c)CF₃ |
| H | C(CH₃)₂CH₂S(O)Et | CF₃ | L-45f |
| H | C(CH₃)₂CH₂S(O)Et | CF₃ | (L-46c)CF₃ |
| H | C(CH₃)₂CH₂S(O)Et | CF₃ | L-46d |
| H | C(CH₃)₂CH₂SO₂Et | CF₃ | CH═CH(Ph-4-Cl) |
| H | C(CH₃)₂CH₂SO₂Et | CF₃ | Ph-4-Cl |
| H | C(CH₃)₂CH₂SO₂Et | CF₃ | Ph-4-Br |
| H | C(CH₃)₂CH₂SO₂Et | CF₃ | Ph-4-CF₃ |
| H | C(CH₃)₂CH₂SO₂Et | CF₃ | Ph-4-OCF₃ |
| H | C(CH₃)₂CH₂SO₂Et | CF₃ | Ph-4-OCF₂Br |
| H | C(CH₃)₂CH₂SO₂Et | CF₃ | Ph-4-OCF₂CHFCF₃ |
| H | C(CH₃)₂CH₂SO₂Et | CF₃ | Ph-4-OCF₂CHFOCF₃ |
| H | C(CH₃)₂CH₂SO₂Et | CF₃ | Ph-4-OCF₂CHFOCF₂CF₂CF₃ |
| H | C(CH₃)₂CH₂SO₂Et | CF₃ | Ph-4-O(L-45g) |
| H | C(CH₃)₂CH₂SO₂Et | CF₃ | Ph-3,4-Cl₂ |
| H | C(CH₃)₂CH₂SO₂Et | CF₃ | Ph(-3-OCF₂O-4-) |
| H | C(CH₃)₂CH₂SO₂Et | CF₃ | Ph(-3-OCF₂CF₂O-4-) |
| H | C(CH₃)₂CH₂SO₂Et | CF₃ | (L-45c)CF₃ |
| H | C(CH₃)₂CH₂SO₂Et | CF₃ | L-45f |
| H | C(CH₃)₂CH₂SO₂Et | CF₃ | (L-46c)CF₃ |
| H | C(CH₃)₂CH₂SO₂Et | CF₃ | L-46d |
| H | C(CH₃)₂CH₂SPr-n | CF₃ | Ph-4-Br |
| H | C(CH₃)₂CH₂SPr-n | CF₃ | Ph-4-CF₃ |
| H | C(CH₃)₂CH₂SPr-n | CF₃ | Ph-4-OCF₃ |
| H | C(CH₃)₂CH₂SPr-n | CF₃ | (L-45c)CF₃ |
| H | C(CH₃)₂CH₂S(O)Pr-n | CF₃ | Ph-4-OCF₃ |
| H | C(CH₃)₂CH₂SO₂Pr-n | CF₃ | Ph-4-Br |
| H | C(CH₃)₂CH₂SO₂Pr-n | CF₃ | Ph-4-CF₃ |
| H | C(CH₃)₂CH₂SO₂Pr-n | CF₃ | Ph-4-OCF₃ |
| H | C(CH₃)₂CH₂SO₂Pr-n | CF₃ | (L-45c)CF₃ |
| H | C(CH₃)₂CH₂SPr-i | CF₃ | Ph-4-Br |
| H | C(CH₃)₂CH₂SPr-i | CF₃ | Ph-4-CF₃ |
| H | C(CH₃)₂CH₂SPr-i | CF₃ | Ph-4-OCF₃ |
| H | C(CH₃)₂CH₂SPr-i | CF₃ | (L-45c)CF₃ |
| H | C(CH₃)₂CH₂S(O)Pr-i | CF₃ | (L-45c)CF₃ |
| H | C(CH₃)₂CH₂SO₂Pr-i | CF₃ | Ph-4-Br |
| H | C(CH₃)₂CH₂SBu-t | CF₃ | Ph-4-CF₃ |
| H | C(CH₃)₂CH₂S(O)Bu-t | CF₃ | Ph-4-OCF₃ |
| H | C(CH₃)₂CH₂SCH₂(Ph-4-Cl) | CF₃ | (L-45c)CF₃ |
| H | C(CH₃)₂CH₂SO₂NH₂ | CF₃ | Ph-4-Br |
| H | C(CH₃)₂CH₂SO₂NHCH₃ | CF₃ | Ph-4-CF₃ |
| H | C(CH₃)₂CH₂SO₂NHEt | CF₃ | Ph-4-Br |
| H | C(CH₃)₂CH₂SO₂NHEt | CF₃ | Ph-4-CF₃ |
| H | C(CH₃)₂CH₂SO₂NHEt | CF₃ | Ph-4-OCF₃ |
| H | C(CH₃)₂CH₂SO₂NHEt | CF₃ | (L-45c)CF₃ |
| H | C(CH₃)₂CH₂SO₂NHCH₂CH₂OCH₃ | CF₃ | Ph-4-OCF₃ |
| H | C(CH₃)₂CH₂SO₂NHCH₂CH₂O(O)CH₃ | CF₃ | (L-45c)CF₃ |
| H | C(CH₃)₂CH₂SO₂NHCH₂CH₂SCH | CF₃ | Ph-4-Br |
| H | C(CH₃)₂CH₂SO₂NHCH₂CH₂S(O)CH₃ | CF₃ | Ph-4-CF₃ |
| H | C(CH₃)₂CH₂SO₂NHCH₂CH₂SO₂CH₃ | CF₃ | Ph-4-OCF₃ |
| H | C(CH₃)₂CH₂SO₂NHCH₂CH₂SEt | CF₃ | (L-45c)CF₃ |
| H | C(CH₃)₂CH₂SO₂NHCH₂CH₂S(O)Et | CF₃ | Ph-4-Br |
| H | C(CH₃)₂CH₂SO₂NHCH₂CH₂SO₂Et | CF₃ | Ph-4-CF₃ |
| H | C(CH₃)₂CH₂SO₂NHCH₂CH₂SPh | CF₃ | Ph-4-OCF₃ |
| H | C(CH₃)₂CH₂SO₂NHCH₂CH₂S(O)Ph | CF₃ | (L-45c)CF₃ |
| H | C(CH₃)₂CH₂SO₂NHCH₂CH₂SO₂Ph | CF₃ | Ph-4-Br |
| H | C(CH₃)₂CH₂SO₂NHCH₂Ph | CF₃ | Ph-4-CF₃ |
| H | C(CH₃)₂CH₂SO₂N(CH₃)₂ | CF₃ | Ph-4-Br |
| H | C(CH₃)₂CH₂SO₂N(CH₃)₂ | CF₃ | Ph-4-CF₃ |
| H | C(CH₃)₂CH₂SO₂N(CH₃)₂ | CF₃ | Ph-4-OCF₃ |
| H | C(CH₃)₂CH₂SO₂N(CH₃)₂ | CF₃ | (L-45c)CF₃ |
| H | C(CH₃)₂CH₂SO₂N(Et)₂ | CF₃ | CF₂O(Ph-4-Cl) |
| H | C(CH₃)₂CH₂SO₂N(Et)₂ | CF₃ | CH═CH(Ph-4-Cl) |
| H | C(CH₃)₂CH₂SO₂N(Et)₂ | CF₃ | CH═CH(Ph-4-Br) |
| H | C(CH₃)₂CH₂SO₂N(Et)₂ | CF₃ | CH═CH(Ph-4-CF₃) |
| H | C(CH₃)₂CH₂SO₂N(Et)₂ | CF₃ | CH═CH(Ph-4-OCF₃) |
| H | C(CH₃)₂CH₂SO₂N(Et)₂ | CF₃ | CH═CH(Ph-3,4-Cl₂) |
| H | C(CH₃)₂CH₂SO₂N(Et)₂ | CF₃ | Ph-4-F |
| H | C(CH₃)₂CH₂SO₂N(Et)₂ | CF₃ | Ph-4-Cl |
| H | C(CH₃)₂CH₂SO₂N(Et)₂ | CF₃ | Ph-4-Br |
| H | C(CH₃)₂CH₂SO₂N(Et)₂ | CF₃ | Ph-4-CF₃ |
| H | C(CH₃)₂CH₂SO₂N(Et)₂ | CF₃ | Ph-4-OCF₃ |
| H | C(CH₃)₂CH₂SO₂N(Et)₂ | CF₃ | Ph-4-OCF₂Br |
| H | C(CH₃)₂CH₂SO₂N(Et)₂ | CF₃ | Ph-4-OCF₂CHF₂ |
| H | C(CH₃)₂CH₂SO₂N(Et)₂ | CF₃ | Ph-4-OCF₂CHFCF₃ |
| H | C(CH₃)₂CH₂SO₂N(Et)₂ | CF₃ | Ph-4-OCF₂CHFOCF₃ |
| H | C(CH₃)₂CH₂SO₂N(Et)₂ | CF₃ | Ph-4-OCF₂CHFOCF₂CF₂CF₃ |
| H | C(CH₃)₂CH₂SO₂N(Et)₂ | CF₃ | Ph-4-O(L-45g) |
| H | C(CH₃)₂CH₂SO₂N(Et)₂ | CF₃ | Ph-3,4-F₂ |
| H | C(CH₃)₂CH₂SO₂N(Et)₂ | CF₃ | Ph-3-F-4-Cl |
| H | C(CH₃)₂CH₂SO₂N(Et)₂ | CF₃ | Ph-3,4-Cl₂ |
| H | C(CH₃)₂CH₂SO₂N(Et)₂ | CF₃ | Ph(-3-OCF₂O-4-) |
| H | C(CH₃)₂CH₂SO₂N(Et)₂ | CF₃ | Ph(-3-OCF₂CF₂O-4-) |
| H | C(CH₃)₂CH₂SO₂N(Et)₂ | CF₃ | (L-45c)CF₃ |
| H | C(CH₃)₂CH₂SO₂N(Et)₂ | CF₃ | L-45f |
| H | C(CH₃)₂CH₂SO₂N(Et)₂ | CF₃ | (L-46c)CF₃ |
| H | C(CH₃)₂CH₂SO₂N(Et)₂ | CF₃ | L-46d |
| H | C(CH₃)₂CH₂SO₂(T-19) | CF₃ | Ph-4-OCF₃ |
| H | C(CH₃)₂CH₂SO₂(T-21) | CF₃ | (L-46c)CF₃ |
| H | C(CH₃)₂CH₂SPh | CF₃ | Ph-4-Br |
| H | C(CH₃)₂CH₂S(O)Ph | CF₃ | Ph-4-CF₃ |
| H | C(CH₃)₂CH₂SO₂Ph | CF₃ | Ph-4-OCF₃ |
| H | C(CH₃)₂CH₂S(L-45a) | CF₃ | (L-45c)CF₃ |
| H | C(CH₃)₂CH₂S(O)(L-45a) | CF₃ | Ph-4-Br |
| H | C(CH₃)₂CH₂SO₂(L-45a) | CF₃ | Ph-4-CF₃ |
| H | CH₂CH₂CH₂SCH₃ | CF₃ | Ph-4-OCF₃ |
| H | CH(CH₃)CH₂CH₂SCH₃ | CF₃ | Ph-4-Br |
| H | CH(CH₃)CH₂CH₂SCH₃ | CF₃ | Ph-4-CF₃ |
| H | CH(CH₃)CH₂CH₂SCH₃ | CF₃ | Ph-4-OCF₃ |
| H | CH(CH₃)CH₂CH₂SCH₃ | CF₃ | (L-45c)CF₃ |
| H | CH(CH₃)CH₂CH₂S(O)CH₃ | CF₃ | Ph-4-Br |
| H | CH(CH₃)CH₂CH₂S(O)CH₃ | CF₃ | Ph-4-CF₃ |
| H | CH(CH₃)CH₂CH₂S(O)CH₃ | CF₃ | Ph-4-OCF₃ |
| H | CH(CH₃)CH₂CH₂S(O)CH₃ | CF₃ | (L-45c)CF₃ |
| H | CH(CH₃)CH₂CH₂SO₂CH₃ | CF₃ | Ph-4-Br |
| H | CH(CH₃)CH₂CH₂SO₂CH₃ | CF₃ | Ph-4-CF₃ |
| H | CH(CH₃)CH₂CH₂SO₂CH₃ | CF₃ | Ph-4-OCF₃ |
| H | CH(CH₃)CH₂CH₂SO₂CH₃ | CF₃ | (L-45c)CF₃ |
| H | CH(CH₃)CH₂CH₂SEt | CF₃ | Ph-4-Br |
| H | CH(CH₃)CH₂CH₂SEt | CF₃ | Ph-4-CF₃ |
| H | CH(CH₃)CH₂CH₂SEt | CF₃ | Ph-4-OCF₃ |
| H | CH(CH₃)CH₂CH₂SEt | CF₃ | (L-45c)CF₃ |
| H | CH(CH₃)CH₂CH₂S(O)Et | CF₃ | Ph-4-Br |
| H | CH(CH₃)CH₂CH₂S(O)Et | CF₃ | Ph-4-CF₃ |
| H | CH(CH₃)CH₂CH₂S(O)Et | CF₃ | Ph-4-OCF₃ |
| H | CH(CH₃)CH₂CH₂S(O)Et | CF₃ | (L-45c)CF₃ |
| H | CH(CH₃)CH₂CH₂SO₂Et | CF₃ | Ph-4-Br |
| H | CH(CH₃)CH₂CH₂SO₂Et | CF₃ | Ph-4-CF₃ |
| H | CH(CH₃)CH₂CH₂SO₂Et | CF₃ | Ph-4-OCF₃ |
| H | CH(CH₃)CH₂CH₂SO₂Et | CF₃ | (L-45c)CF₃ |
| H | CH(CH₃)CH₂CH₂SCH₃ | CF₃ | (L-45c)CF₃ |
| H | CH(CH₂OH)CH₂CH₂SCH₃ | CF₃ | Ph-4-Br |
| H | C(CH₃)₂CH₂CH₂SCH₃ | CF₃ | Ph-4-CF₃ |
| H | C(CH₃)₂CH₂CH₂SEt | CF₃ | Ph-4-OCF₃ |
| H | CH(CH₃)(CH₂)₃SCH₃ | CF₃ | Ph-4-Br |
| H | CH(CH₃)(CH₂)₃SCH₃ | CF₃ | Ph-4-CF₃ |
| H | CH(CH₃)(CH₂)₃SCH₃ | CF₃ | Ph-4-OCF₃ |
| H | CH(CH₃)(CH₂)₃SCH₃ | CF₃ | (L-45c)CF₃ |
| H | CH(CH₃)(CH₂)₃S(O)CH₃ | CF₃ | (L-45c)CF₃ |
| H | CH(CH₃)(CH₂)₃SO₂CH₃ | CF₃ | Ph-4-Br |
| H | CH(CH₃)(CH₂)₃SO₂CH₃ | CF₃ | Ph-4-CF₃ |
| H | CH(CH₃)(CH₂)₃SO₂CH₃ | CF₃ | Ph-4-OCF₃ |
| H | CH(CH₃)(CH₂)₃SO₂CH₃ | CF₃ | (L-45c)CF₃ |
| H | CH(CH₃)(CH₂)₃SEt | CF₃ | Ph-4-Br |
| H | CH(CH₃)(CH₂)₃SEt | CF₃ | Ph-4-CF₃ |
| H | CH(CH₃)(CH₂)₃SEt | CF₃ | Ph-4-OCF₃ |
| H | CH(CH₃)(CH₂)₃SEt | CF₃ | (L-45c)CF₃ |
| H | CH(CH₃)(CH₂)₃S(O)Et | CF₃ | Ph-4-Br |
| H | CH(CH₃)(CH₂)₃SO₂Et | CF₃ | Ph-4-Br |
| H | CH(CH₃)(CH₂)₃SO₂Et | CF₃ | Ph-4-CF₃ |
| H | CH(CH₃)(CH₂)₃SO₂Et | CF₃ | Ph-4-OCF₃ |
| H | CH(CH₃)(CH₂)₃SO₂Et | CF₃ | (L-45c)CF₃ |
| H | C(CH₃)₂(CH₂)₃SCH₃ | CF₃ | Ph-4-CF₃ |
| H | C(CH₃)₂(CH₂)₃S(O)CH₃ | CF₃ | Ph-4-OCF₃ |
| H | CH(CH₃)CH₂SO₂CH₃ | CF₃ | (L-45c)CF₃ |
| H | C(CH₃)₂(CH₂)₃SEt | CF₃ | Ph-4-Br |
| H | C(CH₃)₂(CH₂)₃S(O)Et | CF₃ | Ph-4-CF₃ |
| H | C(CH₃)₂(CH₂)₃SO₂Et | CF₃ | Ph-4-OCF₃ |
| H | C(CH₃)₂(CH₂)₃SO₂NHCH₃ | CF₃ | (L-45c)CF₃ |
| H | C(CH₃)₂(CH₂)₃SO₂NHEt | CF₃ | Ph-4-Br |
| H | C(CH₃)₂(CH₂)₃SO₂N(CH₃)₂ | CF₃ | Ph-4-CF₃ |
| H | C(CH₃)₂(CH₂)₃SO₂N(Et)₂ | CF₃ | Ph-4-OCF₃ |
| H | C(CH₃)₂CH₂CH₂CH(CH₃)SCH₃ | CF₃ | (L-45c)CF₃ |
| H | CH(CH₃)(CH₂)₄SCH₃ | CF₃ | Ph-4-Br |
| H | CH(CH₃)(CH₂)₄SO₂CH₃ | CF₃ | Ph-4-CF₃ |
| H | CH(CH₃)(CH₂)₄SEt | CF₃ | Ph-4-OCF₃ |
| H | CH(CH₃)(CH₂)₄S(O)Et | CF₃ | (L-45c)CF₃ |
| H | CH(CH₃)(CH₂)₄SO₂Et | CF₃ | Ph-4-Br |
| H | T-6 | CF₃ | Ph-4-CF₃ |
| H | T-7 | CF₃ | Ph-4-OCF₃ |
| H | T-8 | CF₃ | (L-45c)CF₃ |
| H | T-9 | CF₃ | Ph-4-Br |
| H | T-11 | CF₃ | Ph-4-CF₃ |
| H | T-12 | CF₃ | Ph-4-OCF₃ |
| H | T-13 | CF₃ | (L-45c)CF₃ |
| H | T-14 | CF₃ | Ph-4-Br |
| H | T-15 | CF₃ | Ph-4-CF₃ |
| H | M-8a | CF₃ | Ph-4-OCF₃ |
| H | M-9a | CF₃ | (L-45c)CF₃ |
| H | M-9b | CF₃ | Ph-4-Br |
| H | M-9c | CF₃ | Ph-4-CF₃ |
| H | M-19a | CF₃ | Ph-4-OCF₃ |
| H | CH₂NHC(O)OCH₃ | CF₃ | (L-45c)CF₃ |
| H | CH₂NHC(O)OEt | CF₃ | Ph-4-Br |
| H | CH₂NHC(O)OPr-i | CF₃ | Ph-4-Br |
| H | CH₂NHC(O)OPr-i | CF₃ | Ph-4-CF₃ |
| H | CH₂NHC(O)OPr-i | CF₃ | Ph-4-OCF₃ |
| H | CH₂NHC(O)OPr-i | CF₃ | (L-45c)CF₃ |
| H | CH₂CH₂NHC(O)CH₃ | CF₃ | Ph-4-CF₃ |
| H | CH₂CH₂NHC(O)N(CH₃)₂ | CF₃ | Ph-4-OCF₃ |
| H | CH₂CH₂NHC(O)Ph | CF₃ | (L-45c)CF₃ |
| H | CH₂CH₂N(CH₃)OCH₃ | CF₃ | Ph-4-Br |
| H | CH(CH₃)CH₂NHC(O)CH₃ | CF₃ | Ph-4-Br |
| H | CH(CH₃)CH₂NHC(O)CH₃ | CF₃ | Ph-4-CF₃ |
| H | CH(CH₃)CH₂NHC(O)CH₃ | CF₃ | Ph-4-OCF₃ |
| H | CH(CH₃)CH₂NHC(O)CH₃ | CF₃ | (L-45c)CF₃ |
| H | CH(CH₃)CH₂NHC(O)Et | CF₃ | Ph-4-CF₃ |
| H | CH(CH₃)CH₂NHC(O)OCH₃ | CF₃ | Ph-4-OCF₃ |
| H | CH(CH₃)CH₂NHC(O)N(CH₃)₂ | CF₃ | (L-45c)CF₃ |
| H | CH(CH₃)CH₂NHC(S)NHEt | CF₃ | Ph-4-Br |
| H | CH(CH₃)CH₂NHSO₂CH₃ | CF₃ | Ph-4-CF₃ |
| H | CH(CH₃)CH₂NHSO₂Et | CF₃ | Ph-4-OCF₃ |
| H | CH(CH₃)CH₂NHSO₂Ph | CF₃ | (L-45c)CF₃ |
| H | CH(CH₃)CH₂NHSO₂N(CH₃)₂ | CF₃ | Ph-4-Br |
| H | CH(CH₃)CH₂NHP(S)(OCH₃)₂ | CF₃ | Ph-4-CF₃ |
| H | CH(CH₃)CH₂NHP(S)(OEt)₂ | CF₃ | Ph-4-OCF₃ |
| H | CH(CH₃)CH₂N(CH₃)₂ | CF₃ | (L-45c)CF₃ |
| H | CH(CH₃)CH₂N(CH₃)SO₂CH₃ | CH₃ | Ph-4-Br |
| H | CH(CH₃)CH₂N(CH₃)SO₂CH₃ | CH₃ | Ph-4-CF₃ |
| H | CH(CH₃)CH₂N(CH₃)SO₂CH₃ | CH₃ | Ph-4-OCF₃ |
| H | CH(CH₃)CH₂N(CH₃)SO₂CH₃ | CH₃ | (L-45c)Cl |
| H | CH(CH₃)CH₂N(CH₃)SO₂CH₃ | CH₃ | (L-45c)Br |
| H | CH(CH₃)CH₂N(CH₃)SO₂CH₃ | CH₃ | (L-45c)CF₃ |
| H | CH(CH₃)CH₂N(CH₃)SO₂CH₃ | Et | Ph-4-Br |
| H | CH(CH₃)CH₂N(CH₃)SO₂CH₃ | n-Pr | Ph-4-CF₃ |
| H | CH(CH₃)CH₂N(CH₃)SO₂CH₃ | i-Pr | Ph-4-OCF₃ |
| H | CH(CH₃)CH₂N(CH₃)SO₂CH₃ | c-Pr | (L-45c)CF₃ |
| H | CH(CH₃)CH₂N(CH₃)SO₂CH₃ | CHF₃ | Ph-4-Br |
| H | CH(CH₃)CH₂N(CH₃)SO₂CH₃ | CF₃ | CF₂O(Ph-4-Cl) |
| H | CH(CH₃)CH₂N(CH₃)SO₂CH₃ | CF₃ | CH═CH(Ph-4-Cl) |
| H | CH(CH₃)CH₂N(CH₃)SO₂CH₃ | CF₃ | CH═CH(Ph-4-Br) |
| H | CH(CH₃)CH₂N(CH₃)SO₂CH₃ | CF₃ | CH═CH(Ph-4-CF₃) |
| H | CH(CH₃)CH₂N(CH₃)SO₂CH₃ | CF₃ | CH═CH(Ph-4-OCF₃) |
| H | CH(CH₃)CH₂N(CH₃)SO₂CH₃ | CF₃ | CH═CH(Ph-3,4-Cl₂) |
| H | CH(CH₃)CH₂N(CH₃)SO₂CH₃ | CF₃ | Ph-4-F |
| H | CH(CH₃)CH₂N(CH₃)SO₂CH₃ | CF₃ | Ph-4-Cl |
| H | CH(CH₃)CH₂N(CH₃)SO₂CH₃ | CF₃ | Ph-4-Br |
| H | CH(CH₃)CH₂N(CH₃)SO₂CH₃ | CF₃ | Ph-4-I |
| H | CH(CH₃)CH₂N(CH₃)SO₂CH₃ | CF₃ | Ph-4-CF₃ |
| H | CH(CH₃)CH₂N(CH₃)SO₂CH₃ | CF₃ | Ph-4-OCHF₂ |
| H | CH(CH₃)CH₂N(CH₃)SO₂CH₃ | CF₃ | Ph-4-OCF₃ |
| H | CH(CH₃)CH₂N(CH₃)SO₂CH₃ | CF₃ | Ph-4-OCF₂Br |
| H | CH(CH₃)CH₂N(CH₃)SO₂CH₃ | CF₃ | Ph-4-OCF₂CHF₂ |
| H | CH(CH₃)CH₂N(CH₃)SO₂CH₃ | CF₃ | Ph-4-OCF₂CHFCl |
| H | CH(CH₃)CH₂N(CH₃)SO₂CH₃ | CF₃ | Ph-4-OCF₂CHFCF₃ |
| H | CH(CH₃)CH₂N(CH₃)SO₂CH₃ | CF₃ | Ph-4-OCF₂CHFOCF₃ |
| H | CH(CH₃)CH₂N(CH₃)SO₂CH₃ | CF₃ | Ph-4-OCF₂CHFOCF₂CF₂CF₃ |
| H | CH(CH₃)CH₂N(CH₃)SO₂CH₃ | CF₃ | Ph-4-O(L-45g) |
| H | CH(CH₃)CH₂N(CH₃)SO₂CH₃ | CF₃ | Ph-3,4-F₂ |
| H | CH(CH₃)CH₂N(CH₃)SO₂CH₃ | CF₃ | Ph-3-F-4-Cl |
| H | CH(CH₃)CH₂N(CH₃)SO₂CH₃ | CF₃ | Ph-3,4-Cl₂ |
| H | CH(CH₃)CH₂N(CH₃)SO₂CH₃ | CF₃ | Ph(-3-OCF₂O-4-) |
| H | CH(CH₃)CH₂N(CH₃)SO₂CH₃ | CF₃ | Ph(-3-OCF₂CF₂O-4-) |
| H | CH(CH₃)CH₂N(CH₃)SO₂CH₃ | CF₃ | (L-45c)Cl |
| H | CH(CH₃)CH₂N(CH₃)SO₂CH₃ | CF₃ | (L-45c)Br |
| H | CH(CH₃)CH₂N(CH₃)SO₂CH₃ | CF₃ | (L-45c)CF₃ |
| H | CH(CH₃)CH₂N(CH₃)SO₂CH₃ | CF₃ | L-45e |
| H | CH(CH₃)CH₂N(CH₃)SO₂CH₃ | CF₃ | L-45f |
| H | CH(CH₃)CH₂N(CH₃)SO₂CH₃ | CF₃ | (L-46c)Cl |
| H | CH(CH₃)CH₂N(CH₃)SO₂CH₃ | CF₃ | (L-46c)Br |
| H | CH(CH₃)CH₂N(CH₃)SO₂CH₃ | CF₃ | (L-46c)CF₃ |
| H | CH(CH₃)CH₂N(CH₃)SO₂CH₃ | CF₃ | L-46d |
| H | CH(CH₃)CH₂N(CH₃)SO₂CH₃ | CF₂Cl | Ph-4-CF₃ |
| H | CH(CH₃)CH₂N(CH₃)SO₂CH₃ | CF₂Br | Ph-4-OCF₃ |
| H | CH(CH₃)CH₂N(CH₃)SO₂CH₃ | CF₂CF₃ | (L-45c)CF₃ |
| H | CH(CH₃)CH₂N(CH₃)SO₂CH₃ | CF₂CF₂CF₃ | Ph-4-Br |
| H | CH(CH₃)CH₂N(CH₃)SO₂CH₃ | CF₂OCH₃ | Ph-4-CF₃ |
| H | CH(CH₃)CH₂N(CH₃)SO₂CH₃ | CF₂SCH₃ | Ph-4-OCF₃ |
| H | C(CH₃)₂CH₂NHC(O)CH₃ | CF₃ | (L-45c)CF₃ |
| H | C(CH₃)₂CH₂NHC(O)Et | CF₃ | Ph-4-Br |
| H | C(CH₃)₂CH₂NHC(O)Pr-c | CF₃ | Ph-4-CF₃ |
| H | C(CH₃)₂CH₂NHC(O)Bu-t | CF₃ | Ph-4-OCF₃ |
| H | C(CH₃)₂CH₂NHC(O)CF₃ | CF₃ | (L-45c)CF₃ |
| H | C(CH₃)₂CH₂NHC(O)Ph | CF₃ | Ph-4-Br |
| H | C(CH₃)₂CH₂NHC(O)OCH₃ | CF₃ | CF₂O(Ph-4-Cl) |
| H | C(CH₃)₂CH₂NHC(O)OCH₃ | CF₃ | CH═CH(Ph-4-Cl) |
| H | C(CH₃)₂CH₂NHC(O)OCH₃ | CF₃ | CH═CH(Ph-4-Br) |
| H | C(CH₃)₂CH₂NHC(O)OCH₃ | CF₃ | CH═CH(Ph-4-CF₃) |
| H | C(CH₃)₂CH₂NHC(O)OCH₃ | CF₃ | CH═CH(Ph-4-OCF₃) |
| H | C(CH₃)₂CH₂NHC(O)OCH₃ | CF₃ | CH═CH(Ph-3,4-Cl₂) |
| H | C(CH₃)₂CH₂NHC(O)OCH₃ | CF₃ | Ph-4-F |
| H | C(CH₃)₂CH₂NHC(O)OCH₃ | CF₃ | Ph-4-Cl |
| H | C(CH₃)₂CH₂NHC(O)OCH₃ | CF₃ | Ph-4-Br |
| H | C(CH₃)₂CH₂NHC(O)OCH₃ | CF₃ | Ph-4-CF₃ |
| H | C(CH₃)₂CH₂NHC(O)OCH₃ | CF₃ | Ph-4-OCF₃ |
| H | C(CH₃)₂CH₂NHC(O)OCH₃ | CF₃ | Ph-4-OCF₂Br |
| H | C(CH₃)₂CH₂NHC(O)OCH₃ | CF₃ | Ph-4-OCF₂CHF₂ |
| H | C(CH₃)₂CH₂NHC(O)OCH₃ | CF₃ | Ph-4-OCF₂CHFCF₃ |
| H | C(CH₃)₂CH₂NHC(O)OCH₃ | CF₃ | Ph-4-OCF₂CHFOCF₃ |
| H | C(CH₃)₂CH₂NHC(O)OCH₃ | CF₃ | Ph-4-OCF₂CHFOCF₂CF₂CF₃ |
| H | C(CH₃)₂CH₂NHC(O)OCH₃ | CF₃ | Ph-4-O(L-45g) |
| H | C(CH₃)₂CH₂NHC(O)OCH₃ | CF₃ | Ph-3,4-F₂ |
| H | C(CH₃)₂CH₂NHC(O)OCH₃ | CF₃ | Ph-3-F-4-Cl |
| H | C(CH₃)₂CH₂NHC(O)OCH₃ | CF₃ | Ph-3,4-Cl₂ |
| H | C(CH₃)₂CH₂NHC(O)OCH₃ | CF₃ | Ph(-3-OCF₂O-4-) |
| H | C(CH₃)₂CH₂NHC(O)OCH₃ | CF₃ | Ph(-3-OCF₂CF₂O-4-) |
| H | C(CH₃)₂CH₂NHC(O)OCH₃ | CF₃ | (L-45c)CF₃ |
| H | C(CH₃)₂CH₂NHC(O)OCH₃ | CF₃ | L-45f |
| H | C(CH₃)₂CH₂NHC(O)OCH₃ | CF₃ | (L-46c)CF₃ |
| H | C(CH₃)₂CH₂NHC(O)OCH₃ | CF₃ | L-46d |
| H | C(CH₃)₂CH₂NHC(O)OEt | CF₃ | Ph-4-CF₃ |
| H | C(CH₃)₂CH₂NHC(O)OPr-n | CF₃ | Ph-4-OCF₃ |
| H | CH₂CH₂CH₂NHC(O)OCH₃ | CF₃ | (L-45c)CF₃ |
| H | CH₂CH₂CH₂NHC(O)OBu-t | CF₃ | Ph-4-Br |
| H | CH₂CH₂CH₂N(CH₃)₂ | CF₃ | Ph-4-CF₃ |
| H | CH₂CH₂CH₂N(CH₃)OCH₃ | CF₃ | Ph-4-OCF₃ |
| H | (M-22a)CHO | CF₃ | (L-45c)CF₃ |
| H | (M-22a)C(O)CH₃ | CF₃ | Ph-4-Br |
| H | (M-22a)C(O)OCH₃ | CF₃ | Ph-4-CF₃ |
| H | (M-22a)C(O)OEt | CF₃ | Ph-4-OCF₃ |
| H | (M-22a)C(O)SCH₃ | CF₃ | (L-45c)CF₃ |
| H | CH₂Si(CH₃)₃ | CF₃ | Ph-4-Br |
| H | C(CH₃)₂CHO | CF₃ | Ph-4-CF₃ |
| H | CH(CH₃)C(O)CH₃ | CF₃ | Ph-4-OCF₃ |
| H | CH(CHO)CH₂SO₂CH₃ | CF₃ | (L-45c)CF₃ |
| H | C(CH₃)(CHO)CH₂SO₂CH₃ | CF₃ | Ph-4-Br |
| H | C(CH₃)(CHO)CH₂SO₂Et | CF₃ | Ph-4-CF₃ |
| H | CH₂CH═NOCH₃ | CF₃ | Ph-4-OCF₃ |
| H | CH₂C(Ph)═NOCH₃ | CF₃ | (L-45c)CF₃ |
| H | CH(CH₃)CH═NOCH₃ | CH₃ | Ph-4-Br |
| H | CH(CH₃)CH═NOCH₃ | CH₃ | Ph-4-CF₃ |
| H | CH(CH₃)CH═NOCH₃ | CH₃ | Ph-4-OCF₃ |
| H | CH(CH₃)CH═NOCH₃ | CH₃ | (L-45c)Cl |
| H | CH(CH₃)CH═NOCH₃ | CH₃ | (L-45c)Br |
| H | CH(CH₃)CH═NOCH₃ | CH₃ | (L-45c)CF₃ |
| H | CH(CH₃)CH═NOCH₃ | Et | Ph-4-Br |
| H | CH(CH₃)CH═NOCH₃ | n-Pr | Ph-4-CF₃ |
| H | CH(CH₃)CH═NOCH₃ | i-Pr | Ph-4-OCF₃ |
| H | CH(CH₃)CH═NOCH₃ | c-Pr | (L-45c)CF₃ |
| H | CH(CH₃)CH═NOCH₃ | CHF₂ | Ph-4-Br |
| H | CH(CH₃)CH═NOCH₃ | CF₃ | CF₂O(Ph-4-Cl) |
| H | CH(CH₃)CH═NOCH₃ | CF₃ | CH═CH(Ph-4-Cl) |
| H | CH(CH₃)CH═NOCH₃ | CF₃ | CH═CH(Ph-4-Br) |
| H | CH(CH₃)CH═NOCH₃ | CF₃ | CH═CH(Ph-4-CF₃) |
| H | CH(CH₃)CH═NOCH₃ | CF₃ | CH═CH(Ph-4-OCF₃) |
| H | CH(CH₃)CH═NOCH₃ | CF₃ | CH═CH(Ph-3,4-Cl₂) |
| H | CH(CH₃)CH═NOCH₃ | CF₃ | Ph-4-F |
| H | CH(CH₃)CH═NOCH₃ | CF₃ | Ph-4-Cl |
| H | CH(CH₃)CH═NOCH₃ | CF₃ | Ph-4-Br |
| H | CH(CH₃)CH═NOCH₃ | CF₃ | Ph-4-I |
| H | CH(CH₃)CH═NOCH₃ | CF₃ | Ph-4-CF₃ |
| H | CH(CH₃)CH═NOCH₃ | CF₃ | Ph-4-OCHF₂ |
| H | CH(CH₃)CH═NOCH₃ | CF₃ | Ph-4-OCF₃ |
| H | CH(CH₃)CH═NOCH₃ | CF₃ | Ph-4-OCF₂Br |
| H | CH(CH₃)CH═NOCH₃ | CF₃ | Ph-4-OCF₂CHF₂ |
| H | CH(CH₃)CH═NOCH₃ | CF₃ | Ph-4-OCF₂CHFCl |
| H | CH(CH₃)CH═NOCH₃ | CF₃ | Ph-4-OCF₂CHFCF₃ |
| H | CH(CH₃)CH═NOCH₃ | CF₃ | Ph-4-OCF₂CHFOCF₃ |
| H | CH(CH₃)CH═NOCH₃ | CF₃ | Ph-4-OCF₂CHFOCF₂CF₂CF₃ |
| H | CH(CH₃)CH═NOCH₃ | CF₃ | Ph-4-O(L-45g) |
| H | CH(CH₃)CH═NOCH₃ | CF₃ | Ph-3,4-F₂ |
| H | CH(CH₃)CH═NOCH₃ | CF₃ | Ph-3-F-4-Cl |
| H | CH(CH₃)CH═NOCH₃ | CF₃ | Ph-3,4-Cl₂ |
| H | CH(CH₃)CH═NOCH₃ | CF₃ | Ph(-3-OCF₂O-4-) |
| H | CH(CH₃)CH═NOCH₃ | CF₃ | Ph(-3-OCF₂CF₂O-4-) |
| H | CH(CH₃)CH═NOCH₃ | CF₃ | (L-45c)Cl |
| H | CH(CH₃)CH═NOCH₃ | CF₃ | (L-45c)Br |
| H | CH(CH₃)CH═NOCH₃ | CF₃ | (L-45c)CF₃ |
| H | CH(CH₃)CH═NOCH₃ | CF₃ | L-45e |
| H | CH(CH₃)CH═NOCH₃ | CF₃ | L-45f |
| H | CH(CH₃)CH═NOCH₃ | CF₃ | (L-46c)Cl |
| H | CH(CH₃)CH═NOCH₃ | CF₃ | (L-46c)Br |
| H | CH(CH₃)CH═NOCH₃ | CF₃ | (L-46c)CF₃ |
| H | CH(CH₃)CH═NOCH₃ | CF₃ | L-46d |
| H | CH(CH₃)CH═NOCH₃ | CF₂Cl | Ph-4-CF₃ |
| H | CH(CH₃)CH═NOCH₃ | CF₂Br | Ph-4-OCF₃ |
| H | CH(CH₃)CH═NOCH₃ | CF₂CF₃ | (L-45c)CF₃ |
| H | CH(CH₃)CH═NOCH₃ | CF₂CF₂CF₃ | Ph-4-Br |
| H | CH(CH₃)CH═NOCH₃ | CF₂OCH₃ | Ph-4-CF₃ |
| H | CH(CH₃)CH═NOCH₃ | CF₂SCH₃ | Ph-4-OCF₃ |
| H | CH(CH₃)CH═NOPr-n | CF₃ | Ph-4-Br |
| H | CH(CH₃)CH═NOPr-n | CF₃ | Ph-4-CF₃ |
| H | CH(CH₃)CH═NOPr-n | CF₃ | Ph-4-OCF₃ |
| H | CH(CH₃)CH═NOPr-n | CF₃ | (L-45c)CF₃ |
| H | CH(CH₃)CH═NOCH₂Pr-c | CF₃ | (L-45c)CF₃ |
| H | CH(CH₃)CH═NOCH₂CH₂OEt | CF₃ | Ph-4-Br |
| H | CH(CH₃)CH═NOCH₂CH₂SEt | CF₃ | Ph-4-CF₃ |
| H | CH(CH₃)CH═NOCH₂CH═CH₂ | CF₃ | Ph-4-OCF₃ |
| H | CH(CH₃)CH═NOCH₂Ph | CF₃ | (L-45c)CF₃ |
| H | CH(CH₃)C(CH₃)═NOCH₃ | CF₃ | Ph-4-Br |
| H | C(CH₃)₂CH═NOH | CF₃ | Ph-4-Br |
| H | C(CH₃)₂CH═NOH | CF₃ | Ph-4-CF₃ |
| H | C(CH₃)₂CH═NOH | CF₃ | Ph-4-OCF₃ |
| H | C(CH₃)₂CH═NOH | CF₃ | (L-45c)CF₃ |
| H | C(CH₃)₂CH═NOCH₃ | CF₃ | CF₂O(Ph-4-Cl) |
| H | C(CH₃)₂CH═NOCH₃ | CF₃ | CH═CH(Ph-4-Cl) |
| H | C(CH₃)₂CH═NOCH₃ | CF₃ | CH═CH(Ph-4-Br) |
| H | C(CH₃)₂CH═NOCH₃ | CF₃ | CH═CH(Ph-4-CF₃) |
| H | C(CH₃)₂CH═NOCH₃ | CF₃ | CH═CH(Ph-4-OCF₃) |
| H | C(CH₃)₂CH═NOCH₃ | CF₃ | CH═CH(Ph-3,4-Cl₂) |
| H | C(CH₃)₂CH═NOCH₃ | CF₃ | Ph-4-F |
| H | C(CH₃)₂CH═NOCH₃ | CF₃ | Ph-4-Cl |
| H | C(CH₃)₂CH═NOCH₃ | CF₃ | Ph-4-Br |
| H | C(CH₃)₂CH═NOCH₃ | CF₃ | Ph-4-CF₃ |
| H | C(CH₃)₂CH═NOCH₃ | CF₃ | Ph-4-OCF₃ |
| H | C(CH₃)₂CH═NOCH₃ | CF₃ | Ph-4-OCF₂Br |
| H | C(CH₃)₂CH═NOCH₃ | CF₃ | Ph-4-OCF₂CHF₂ |
| H | C(CH₃)₂CH═NOCH₃ | CF₃ | Ph-4-OCF₂CHFCF₃ |
| H | C(CH₃)₂CH═NOCH₃ | CF₃ | Ph-4-OCF₂CHFOCF₃ |
| H | C(CH₃)₂CH═NOCH₃ | CF₃ | Ph-4-OCF₂CHFOCF₂CF₂CF₃ |
| H | C(CH₃)₂CH═NOCH₃ | CF₃ | Ph-4-O(L-45g) |
| H | C(CH₃)₂CH═NOCH₃ | CF₃ | Ph-3,4-F₂ |
| H | C(CH₃)₂CH═NOCH₃ | CF₃ | Ph-3-F-4-Cl |
| H | C(CH₃)₂CH═NOCH₃ | CF₃ | Ph-3,4-Cl₂ |
| H | C(CH₃)₂CH═NOCH₃ | CF₃ | Ph(-3-OCF₂O-4-) |
| H | C(CH₃)₂CH═NOCH₃ | CF₃ | Ph(-3-OCF₂CF₂O-4-) |
| H | C(CH₃)₂CH═NOCH₃ | CF₃ | (L-45c)CF₃ |
| H | C(CH₃)₂CH═NOCH₃ | CF₃ | L-45f |
| H | C(CH₃)₂CH═NOCH₃ | CF₃ | (L-46c)CF₃ |
| H | C(CH₃)₂CH═NOCH₃ | CF₃ | L-46d |
| H | C(CH₃)₂CH═NOCH₃ | CF₃ | Ph-4-CF₃ |
| H | C(CH₃)₂CH═NOCH₂C(O)OBu-t | CF₃ | Ph-4-OCF₃ |
| H | C(CH₃)₂CH═NOCH₂C(O)N(Et) | CF₃ | (L-45c)CF₃ |
| H | C(CH₃)(CH₂SO₂CH₃)CH═NOH | CF₃ | Ph-4-Br |
| H | C(CH₃)(CH₂SO₂Et)CH═NOH | CF₃ | Ph-4-CF₃ |
| H | CH₂CH₂CH═NOCH₃ | CF₃ | Ph-4-OCF₃ |
| H | CH(CH₃)CH₂CH═NOCH₃ | CF₃ | (L-45c)CF₃ |
| H | CH₂CH₂CH₂CH═NOEt | CF₃ | Ph-4-Br |
| H | CH₂C(O)OEt | CF₃ | Ph-4-CF₃ |
| H | CH(CH₃)C(O)OCH₃ | CF₃ | Ph-4-OCF₃ |
| H | CH(CH₃)C(O)OEt | CF₃ | (L-45c)CF₃ |
| H | CH₂CH₂C(O)OEt | CF₃ | Ph-4-Br |
| H | CH(CH₃)CH₂C(O)OEt | CF₃ | Ph-4-CF₃ |
| H | CH(CH₃)C(O)NHEt | CF₃ | Ph-4-OCF₃ |
| H | CH(CH₃)C(O)NHPr-n | CF₃ | (L-45c)CF₃ |
| H | CH(CH₃)C(O)NHBu-n | CF₃ | Ph-4-Br |
| H | CH(CH₃)C(O)NHCH₂Ph | CF₃ | Ph-4-CF₃ |
| H | CH(CH₃)C(O)N(CH₃)₂ | CF₃ | Ph-4-OCF₃ |
| H | CH(CH₃)C(O)N(Et)₂ | CF₃ | (L-45c)CF₃ |
| H | CH(CH₃)C(O)N(Pr-n)₂ | CF₃ | Ph-4-Br |
| H | CH(CH₃)C(O)(T-16) | CF₃ | Ph-4-CF₃ |
| H | CH(CH₃)C(O)(T-17) | CF₃ | Ph-4-OCF₃ |
| H | CH(CH₃)C(O)(T-18) | CF₃ | (L-45c)CF₃ |
| H | CH(CH₃)C(O)(T-19) | CF₃ | Ph-4-Br |
| H | CH(CH₃)C(O)(T-20) | CF₃ | Ph-4-CF₃ |
| H | CH(CH₃)C(O)(T-21) | CF₃ | Ph-4-OCF₃ |
| H | CH(CH₃)CH₂C(O)NHCH₃ | CF₃ | (L-45c)CF₃ |
| H | CH(CH₃)CH₂C(O)NHEt | CF₃ | Ph-4-Br |
| H | CH(CH₃)CH₂C(O)N(CH₃)₂ | CF₃ | Ph-4-CF₃ |
| H | CH(CH₃)CH₂C(O)N(Et)₂ | CF₃ | Ph-4-OCF₃ |
| H | CH(CH₃)CH₂C(O)N(CH₃)Ph | CF₃ | (L-45c)CF₃ |
| H | CH(CH₃)CN | CF₃ | Ph-4-Br |
| H | C(CH₃)₂CN | CF₃ | Ph-4-CF₃ |
| H | CH₂CH═CH₂ | CF₃ | Ph-4-OCF₃ |
| CH₂CH═CH₂CH₂CH═CH₂ | CF₃ | (L-45c)CF₃ | |
| H | C(CH₃)₂CH═CH₂ | CF₃ | CH═CH(Ph-4-Cl) |
| H | C(CH₃)₂CH═CH₂ | CF₃ | Ph-4-Cl |
| H | C(CH₃)₂CH═CH₂ | CF₃ | Ph-4-Br |
| H | C(CH₃)₂CH═CH₂ | CF₃ | Ph-4-CF₃ |
| H | C(CH₃)₂CH═CH₂ | CF₃ | Ph-4-OCF₃ |
| H | C(CH₃)₂CH═CH₂ | CF₃ | Ph-4-OCF₂Br |
| H | C(CH₃)₂CH═CH₂ | CF₃ | Ph-4-OCF₂CHFCF₃ |
| H | C(CH₃)₂CH═CH₂ | CF₃ | Ph-4-OCF₂CHFOCF₃ |
| H | C(CH₃)₂CH═CH₂ | CF₃ | Ph-4-OCF₂CHFOCF₂CF₂CF₃ |
| H | C(CH₃)₂CH═CH₂ | CF₃ | Ph-4-O(L-45g) |
| H | C(CH₃)₂CH═CH₂ | CF₃ | Ph-3,4-Cl₂ |
| H | C(CH₃)₂CH═CH₂ | CF₃ | Ph(-3-OCF₂O-4-) |
| H | C(CH₃)₂CH═CH₂ | CF₃ | Ph(-3-OCF₂CF₂O-4-) |
| H | C(CH₃)₂CH═CH₂ | CF₃ | (L-45c)CF₃ |
| H | C(CH₃)₂CH═CH₂ | CF₃ | L-45f |
| H | C(CH₃)₂CH═CH₂ | CF₃ | (L-46c)CF₃ |
| H | C(CH₃)₂CH═CH₂ | CF₃ | L-46d |
| H | C(CH₃)₂CH═CHC(O)NHEt | CF₃ | Ph-4-Br |
| H | C(CH₃)₂CH═CHPh(E) | CF₃ | Ph-4-CF₃ |
| H | CH₂C≡CH | CF₃ | Ph-4-OCF₃ |
| CH₃ | CH₂C≡CH | CF₃ | Ph-4-Br |
| CH₃ | CH₂C≡CH | CF₃ | Ph-4-CF₃ |
| CH₃ | CH₂C≡CH | CF₃ | Ph-4-OCF₃ |
| CH₃ | CH₂C≡CH | CF₃ | (L-45c)CF₃ |
| H | C(CH₃)₂C≡CH | CF₃ | CH═CH(Ph-4-Cl) |
| H | C(CH₃)₂C≡CH | CF₃ | Ph-4-Cl |
| H | C(CH₃)₂C≡CH | CF₃ | Ph-4-Br |
| H | C(CH₃)₂C≡CH | CF₃ | Ph-4-CF₃ |
| H | C(CH₃)₂C≡CH | CF₃ | Ph-4-OCF₃ |
| H | C(CH₃)₂C≡CH | CF₃ | Ph-4-OCF₂Br |
| H | C(CH₃)₂C≡CH | CF₃ | Ph-4-OCF₂CHFCF₃ |
| H | C(CH₃)₂C≡CH | CF₃ | Ph-4-OCF₂CHFOCF₃ |
| H | C(CH₃)₂C≡CH | CF₃ | Ph-4-OCF₂CHFOCF₂CF₂CF₃ |
| H | C(CH₃)₂C≡CH | CF₃ | Ph-4-O(L-45g) |
| H | C(CH₃)₂C≡CH | CF₃ | Ph-3,4-Cl₂ |
| H | C(CH₃)₂C≡CH | CF₃ | Ph(-3-OCF₂O-4-) |
| H | C(CH₃)₂C≡CH | CF₃ | Ph(-3-OCF₂CF₂O-4-) |
| H | C(CH₃)₂C≡CH | CF₃ | (L-45c)CF₃ |
| H | C(CH₃)₂C≡CH | CF₃ | L-45f |
| H | C(CH₃)₂C≡CH | CF₃ | (L-46c)CF₃ |
| H | C(CH₃)₂C≡CH | CF₃ | L-46d |
| H | C(CH₃)₂C≡CPh | CF₃ | (L-45c)CF₃ |
| H | C(CH₃)₂C≡C(Ph-4-CH₃) | CF₃ | Ph-4-Br |
| H | C(CH₃)₂C≡C(Ph-4-CF₃) | CF₃ | Ph-4-CF₃ |
| H | C(CH₃)₂C≡C(Ph-4-OCH₃) | CF₃ | Ph-4-OCF₃ |
| H | C(CH₃)₂C≡C(Ph-4-OCH₃) | CF₃ | (L-45c)CF₃ |
| H | C(CH₃)₂C≡C(Ph-2,4-F₂) | CF₃ | Ph-4-Br |
| H | C(CH₃)₂C≡C(Ph-2,4-Cl₂) | CF₃ | Ph-4-CF₃ |
| H | C(CH₃)₂C≡C(Ph-2,6-Cl₂) | CF₃ | Ph-4-OCF₃ |
| H | C(CH₃)₂C≡C(1-Naph) | CF₃ | (L-45c)CF₃ |
| H | C(CH₃)₂C≡C(L-3a) | CF₃ | Ph-4-Br |
| H | C(CH₃)₂C≡C(L-4a) | CF₃ | Ph-4-CF₃ |
| H | C(CH₃)₂C≡C(L-45a) | CF₃ | Ph-4-OCF₃ |
| H | C(CH₃)₂C≡C(L-45g) | CF₃ | (L-45c)CF₃ |
| H | C(CH₃)₂C≡C(L-46a) | CF₃ | Ph-4-Br |
| H | CH₂Ph | CF₃ | Ph-4-CF₃ |
| CH₃ | CH₂Ph | CF₃ | Ph-4-OCF₃ |
| Et | CH₂Ph | CF₃ | (L-45c)CF₃ |
| H | CH₂(Ph-2-F) | CF₃ | Ph-4-Br |
| CH₃ | CH₂(Ph-2-F) | CF₃ | Ph-4-CF₃ |
| H | CH₂(Ph-2-Cl) | CF₃ | Ph-4-OCF₃ |
| H | CH₂(Ph-3-Cl) | CF₃ | (L-45c)CF₃ |
| CH₃ | CH₂(Ph-3-Cl) | CF₃ | Ph-4-Br |
| H | CH₂(Ph-4-Cl) | CF₃ | Ph-4-CF₃ |
| H | CH₂(Ph-2-CH₃) | CF₃ | Ph-4-OCF₃ |
| H | CH₂(Ph-3-CH₃) | CF₃ | (L-45c)CF₃ |
| H | CH₂(Ph-4-CH₃) | CF₃ | Ph-4-Br |
| H | CH₂(Ph-2-CF₃) | CF₃ | Ph-4-CF₃ |
| H | CH₂(Ph-2-OCH₃) | CF₃ | Ph-4-OCF₃ |
| H | CH₂(Ph-3-OCH₃) | CF₃ | (L-45c)CF₃ |
| H | CH₂(Ph-4-OCH₃) | CF₃ | Ph-4-Br |
| H | CH₂(Ph-4-OCF₃) | CF₃ | Ph-4-CF₃ |
| H | CH₂(Ph-2,3-Cl₂) | CF₃ | Ph-4-OCF₃ |
| H | CH₂(Ph-2,4-Cl₂) | CF₃ | (L-45c)CF₃ |
| H | CH₂(Ph-3,4-Cl₂) | CF₃ | Ph-4-Br |
| H | CH₂(L-45a) | CF₃ | Ph-4-CF₃ |
| H | CH₂(L-46a) | CF₃ | Ph-4-OCF₃ |
| H | CH₂(L-47a) | CF₃ | (L-45c)CF₃ |
| H | CH(CH₃)Ph | CF₃ | Ph-4-Br |
| H | CH(CH₃)Ph(R) | CF₃ | Ph-4-CF₃ |
| H | CH(CH₃)Ph(S) | CF₃ | Ph-4-OCF₃ |
| H | CH(CH₃)(Ph-2-Cl) | CF₃ | (L-45c)CF₃ |
| H | CH(CH₃)(Ph-3-Cl) | CF₃ | Ph-4-Br |
| H | CH(CH₃)(Ph-4-Cl) | CF₃ | Ph-4-CF₃ |
| H | CH(CH₃)(L-1a) | CF₃ | Ph-4-OCF₃ |
| H | CH(CH₃)(L-3a) | CF₃ | (L-45c)CF₃ |
| H | CH(CH₃)(L-45a) | CF₃ | Ph-4-Br |
| H | C(CH₃)₂Ph | CF₃ | Ph-4-CF₃ |
| H | C(CH₃)₂(Ph-3-Cl) | CF₃ | Ph-4-OCF₃ |
| H | C(CH₃)₂(Ph-4-Cl) | CF₃ | (L-45c)CF₃ |
| H | CH₂CH₂Ph | CF₃ | Ph-4-Br |
| H | CH₂CH₂(Ph-2-Cl) | CF₃ | Ph-4-CF₃ |
| H | CH₂CH₂(Ph-3-Cl) | CF₃ | Ph-4-OCF₃ |
| H | CH₂CH₂(Ph-4-Cl) | CF₃ | (L-45c)CF₃ |
| H | CH₂CH₂(L-46a) | CF₃ | Ph-4-Br |
| H | C(CH₃)₂CH₂Ph | CF₃ | Ph-4-CF₃ |
| H | CH₂CH₂CH₂Ph | CF₃ | Ph-4-OCF₃ |
| H | OCH₃ | CF₃ | (L-45c)CF₃ |
| H | OPr-n | CF₃ | Ph-4-Br |
| H | OCH₂CH═CHCl | CF₃ | Ph-4-CF₃ |
| H | OCH₂Ph | CF₃ | Ph-4-OCF₃ |
| H | NHCHO | CF₃ | (L-45c)CF₃ |
| H | NHC(O)CH₃ | CF₃ | Ph-4-Br |
| H | NHC(O)Ph | CF₃ | Ph-4-CF₃ |
| H | NHC(O)OCH₃ | CF₃ | Ph-4-OCF₃ |
| H | NHC(O)OPh | CF₃ | (L-45c)CF₃ |
| H | NHC(O)OCH₂Ph | CF₃ | Ph-4-Br |
| H | N(CH₃)₂ | CF₃ | Ph-4-CF₃ |
| H | N(CH₃)CHO | CF₃ | Ph-4-OCF₃ |
| H | N(CH₃)C(O)CH₃ | CF₃ | (L-45c)CF₃ |
| H | N(CH₃)C(O)OCH₃ | CF₃ | Ph-4-Br |

TABLE 3
In the table, the number(s) showing the position(s) of the substituent (X)ₘ and (Y)ₙ correspond to the number(s)
shown in the following structural formulae, and the symbol — means unsubstituted.

| | |
|---|---|
| 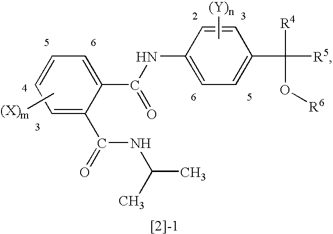 | 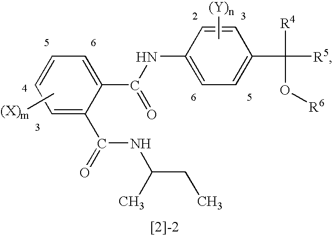 |
| 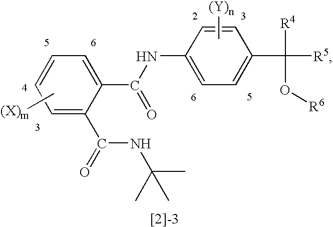 | 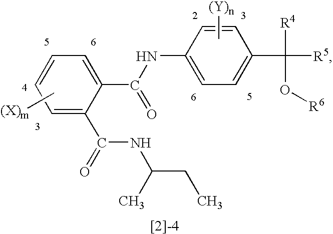 |
| 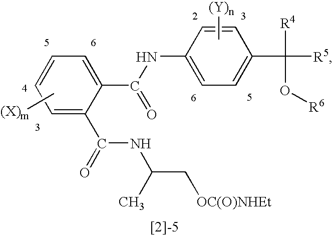 | 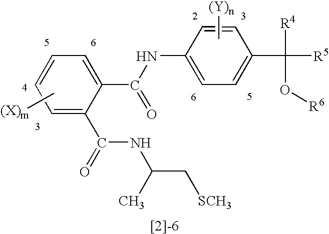 |
| 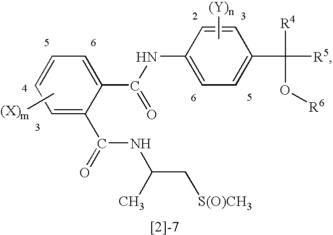 | 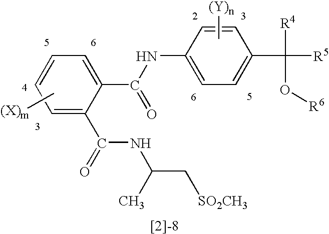 |
| 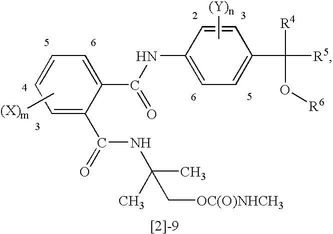 | 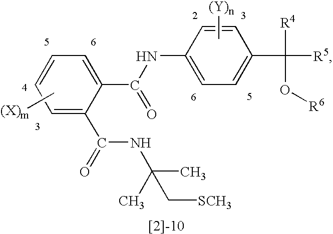 |
| 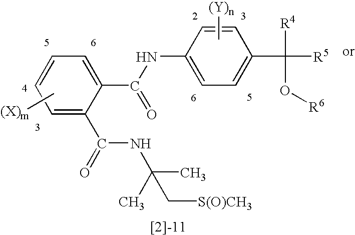 | 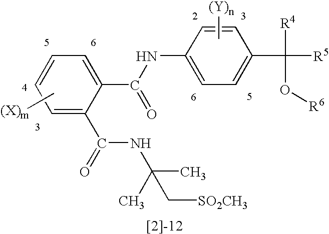 |

| (X)ₘ | (Y)ₙ | R⁴ | R⁵ | R⁶ |
|---|---|---|---|---|
| — | 2-CH₃ | CF₃ | Ph-4-Br | H |
| 5-F | 2-CH₃ | CF₃ | Ph-4-CF₃ | H |
| 6-F | 2-CH₃ | CF₃ | Ph-4-OCF₃ | H |
| 3,4-F₂ | 2-CH₃ | CF₃ | Ph-4-Br | H |
| 3,4-F₂ | 2-CH₃ | CF₃ | Ph-4-CF₃ | H |
| 3,4-F₂ | 2-CH₃ | CF₃ | Ph-4-OCF₃ | H |
| 3,4-F₂ | 2-CH₃ | CF₃ | (L-45c)CF₃ | H |
| 3,6-F₂ | 2-CH₃ | CF₃ | (L-45c)CF₃ | H |
| 4,5-F₂ | 2-CH₃ | CF₃ | Ph-4-Br | H |
| 4-Cl | 2-CH₃ | CF₃ | Ph-4-CF₃ | H |
| 5-Cl | 2-CH₃ | CF₃ | Ph-4-OCF₃ | H |
| 6-Cl | 2-CH₃ | CF₃ | (L-45c)CF₃ | H |
| 3-Cl-4-F | 2-CH₃ | CF₃ | Ph-4-Br | H |
| 3-Cl-4-F | 2-CH₃ | CF₃ | Ph-4-CF₃ | H |
| 3-Cl-4-F | 2-CH₃ | CF₃ | Ph-4-OCF₃ | H |
| 3-Cl-4-F | 2-CH₃ | CF₃ | (L-45c)CF₃ | H |
| 3,4-Cl₂ | 2-CH₃ | CH₃ | Ph-4-Br | H |
| 3,4-Cl₂ | 2-CH₃ | CH₃ | Ph-4-CF₃ | H |
| 3,4-Cl₂ | 2-CH₃ | CH₃ | Ph-4-OCF₃ | H |
| 3,4-Cl₂ | 2-CH₃ | CH₃ | (L-45c)Cl | H |
| 3,4-Cl₂ | 2-CH₃ | CH₃ | (L-45c)Br | H |
| 3,4-Cl₂ | 2-CH₃ | CH₃ | (L-45c)CF₃ | H |
| 3,4-Cl₂ | 2-CH₃ | CH₃ | (L-45c)CF₃ | H |
| 3,4-Cl₂ | 2-CH₃ | n-Pr | Ph-4-Br | H |
| 3,4-Cl₂ | 2-CH₃ | i-Pr | Ph-4-CF₃ | H |
| 3,4-Cl₂ | 2-CH₃ | c-Pr | Ph-4-OCF₃ | H |
| 3,4-Cl₂ | 2-CH₃ | CHF₂ | (L-45c)CF₃ | H |
| 3,4-Cl₂ | 2-CH₃ | CF₃ | CF₂O(Ph-4-Cl) | H |
| 3,4-Cl₂ | 2-CH₃ | CF₃ | CH═CH(Ph-4-Cl) | H |
| 3,4-Cl₂ | 2-CH₃ | CF₃ | CH═CH(Ph-4-Br) | H |
| 3,4-Cl₂ | 2-CH₃ | CF₃ | CH═CH(Ph-4-CF₃) | H |
| 3,4-Cl₂ | 2-CH₃ | CF₃ | CH═CH(Ph-4-OCF₃) | H |
| 3,4-Cl₂ | 2-CH₃ | CF₃ | CH═CH(Ph-3,4-Cl₂) | H |
| 3,4-Cl₂ | 2-CH₃ | CF₃ | Ph-4-F | H |
| 3,4-Cl₂ | 2-CH₃ | CF₃ | Ph-4-Cl | H |
| 3,4-Cl₂ | 2-CH₃ | CF₃ | Ph-4-Br | H |
| 3,4-Cl₂ | 2-CH₃ | CF₃ | Ph-4-I | H |
| 3,4-Cl₂ | 2-CH₃ | CF₃ | Ph-4-CF₃ | H |
| 3,4-Cl₂ | 2-CH₃ | CF₃ | Ph-4-OCHF₂ | H |
| 3,4-Cl₂ | 2-CH₃ | CF₃ | Ph-4-OCF₃ | H |
| 3,4-Cl₂ | 2-CH₃ | CF₃ | Ph-4-OCF₂Br | H |
| 3,4-Cl₂ | 2-CH₃ | CF₃ | Ph-4-OCF₂CHF₂ | H |
| 3,4-Cl₂ | 2-CH₃ | CF₃ | Ph-4-OCF₂CHFCl | H |
| 3,4-Cl₂ | 2-CH₃ | CF₃ | Ph-4-OCF₂CHFCF₃ | H |
| 3,4-Cl₂ | 2-CH₃ | CF₃ | Ph-4-OCF₂CHFOCF₃ | H |
| 3,4-Cl₂ | 2-CH₃ | CF₃ | Ph-4-OCF₂CHFOCF₂CF₂CF₃ | H |
| 3,4-Cl₂ | 2-CH₃ | CF₃ | Ph-4-O(L-45g) | H |
| 3,4-Cl₂ | 2-CH₃ | CF₃ | Ph-3,4-F₂ | H |
| 3,4-Cl₂ | 2-CH₃ | CF₃ | Ph-3-F-4-Cl | H |
| 3,4-Cl₂ | 2-CH₃ | CF₃ | Ph-3,4-Cl₂ | H |
| 3,4-Cl₂ | 2-CH₃ | CF₃ | Ph(-3-OCF₂O-4-) | H |
| 3,4-Cl₂ | 2-CH₃ | CF₃ | Ph(-3-OCF₂CF₂O-4-) | H |
| 3,4-Cl₂ | 2-CH₃ | CF₃ | (L-45c)Cl | H |
| 3,4-Cl₂ | 2-CH₃ | CF₃ | (L-45c)Br | H |
| 3,4-Cl₂ | 2-CH₃ | CF₃ | (L-45c)CF₃ | H |
| 3,4-Cl₂ | 2-CH₃ | CF₃ | L-45e | H |
| 3,4-Cl₂ | 2-CH₃ | CF₃ | L-45f | H |
| 3,4-Cl₂ | 2-CH₃ | CF₃ | (L-46c)Cl | H |
| 3,4-Cl₂ | 2-CH₃ | CF₃ | (L-46c)Br | H |
| 3,4-Cl₂ | 2-CH₃ | CF₃ | (L-46c)CF₃ | H |
| 3,4-Cl₂ | 2-CH₃ | CF₃ | L-46d | H |
| 3,4-Cl₂ | 2-CH₃ | CF₂Cl | Ph-4-Br | H |
| 3,4-Cl₂ | 2-CH₃ | CF₂Br | Ph-4-CF₃ | H |
| 3,4-Cl₂ | 2-CH₃ | CF₂CF₃ | Ph-4-OCF₃ | H |
| 3,4-Cl₂ | 2-CH₃ | CF₂CF₂CF₃ | (L-45c)CF₃ | H |
| 3,4-Cl₂ | 2-CH₃ | CF₂OCH₃ | Ph-4-Br | H |
| 3,4-Cl₂ | 2-CH₃ | CF₂SCH₃ | Ph-4-CF₃ | H |
| 3,5-Cl₂ | 2-CH₃ | CF₃ | Ph-4-OCF₃ | H |
| 3,6-Cl₂ | 2-CH₃ | CF₃ | (L-45c)CF₃ | H |
| 5,6-Cl₂ | 2-CH₃ | CF₃ | Ph-4-Br | H |
| 6-Br | 2-CH₃ | CF₃ | Ph-4-CF₃ | H |
| 3,4-Br₂ | 2-CH₃ | CF₃ | Ph-4-OCF₃ | H |
| 3,6-Br₂ | 2-CH₃ | CF₃ | (L-45c)CF₃ | H |
| 5,6-Br₂ | 2-CH₃ | CF₃ | Ph-4-Br | H |
| 3-I | 2-I | CF₃ | Ph-4-CF₃ | H |
| 3-I | 2-CH₃ | CF₃ | CF₂O(Ph-4-Cl) | CH₃ |
| 3-I | 2-CH₃ | CF₃ | CF₂O(Ph-4-Cl) | CH₂OCH₃ |
| 3-I | 2-CH₃ | CF₃ | CF₂O(Ph-4-Cl) | C(O)CH₃ |
| 3-I | 2-CH₃ | CF₃ | CF₂O(Ph-4-Cl) | Si(CH₃)₃ |
| 3-I | 2-CH₃ | CF₃ | CH═CH(Ph-4-Cl) | CH₃ |
| 3-I | 2-CH₃ | CF₃ | CH═CH(Ph-4-Cl) | CH₂OCH₃ |
| 3-I | 2-CH₃ | CF₃ | CH═CH(Ph-4-Cl) | C(O)CH₃ |
| 3-I | 2-CH₃ | CF₃ | CH═CH(Ph-4-Cl) | Si(CH₃)₃ |
| 3-I | 2-CH₃ | CF₃ | CH═CH(Ph-4-Br) | CH₃ |
| 3-I | 2-CH₃ | CF₃ | CH═CH(Ph-4-Br) | CH₂OCH₃ |
| 3-I | 2-CH₃ | CF₃ | CH═CH(Ph-4-Br) | C(O)CH₃ |
| 3-I | 2-CH₃ | CF₃ | CH═CH(Ph-4-Br) | Si(CH₃)₃ |
| 3-I | 2-CH₃ | CF₃ | CH═CH(Ph-4-CF₃) | CH₃ |
| 3-I | 2-CH₃ | CF₃ | CH═CH(Ph-4-CF₃) | CH₂OCH₃ |
| 3-I | 2-CH₃ | CF₃ | CH═CH(Ph-4-CF₃) | C(O)CH₃ |
| 3-I | 2-CH₃ | CF₃ | CH═CH(Ph-4-CF₃) | Si(CH₃)₃ |
| 3-I | 2-CH₃ | CF₃ | CH═CH(Ph-4-OCF₃) | CH₃ |
| 3-I | 2-CH₃ | CF₃ | CH═CH(Ph-4-OCF₃) | CH₂OCH₃ |
| 3-I | 2-CH₃ | CF₃ | CH═CH(Ph-4-OCF₃) | C(O)CH₃ |
| 3-I | 2-CH₃ | CF₃ | CH═CH(Ph-4-OCF₃) | Si(CH₃)₃ |
| 3-I | 2-CH₃ | CF₃ | CH═CH(Ph-3,4-Cl₂) | CH₃ |
| 3-I | 2-CH₃ | CF₃ | CH═CH(Ph-3,4-Cl₂) | CH₂OCH₃ |
| 3-I | 2-CH₃ | CF₃ | CH═CH(Ph-3,4-Cl₂) | C(O)CH₃ |
| 3-I | 2-CH₃ | CF₃ | CH═CH(Ph-3,4-Cl₂) | Si(CH₃)₃ |
| 3-I | 2-CH₃ | CF₃ | Ph-4-F | CH₃ |
| 3-I | 2-CH₃ | CF₃ | Ph-4-F | CH₂OCH₃ |
| 3-I | 2-CH₃ | CF₃ | Ph-4-F | C(O)CH₃ |
| 3-I | 2-CH₃ | CF₃ | Ph-4-F | Si(CH₃)₃ |
| 3-I | 2-CH₃ | CF₃ | Ph-4-Cl | CH₃ |
| 3-I | 2-CH₃ | CF₃ | Ph-4-Cl | CH₂OCH₃ |
| 3-I | 2-CH₃ | CF₃ | Ph-4-Cl | C(O)CH₃ |
| 3-I | 2-CH₃ | CF₃ | Ph-4-Cl | Si(CH₃)₃ |
| 3-I | 2-CH₃ | CF₃ | Ph-4-Br | CH₃ |
| 3-I | 2-CH₃ | CF₃ | Ph-4-Br | Et |
| 3-I | 2-CH₃ | CF₃ | Ph-4-Br | n-Pr |
| 3-I | 2-CH₃ | CF₃ | Ph-4-Br | i-Pr |
| 3-I | 2-CH₃ | CF₃ | Ph-4-Br | n-Bu |
| 3-I | 2-CH₃ | CF₃ | Ph-4-Br | CH₂CF₃ |
| 3-I | 2-CH₃ | CF₃ | Ph-4-Br | CH₂OCH₃ |
| 3-I | 2-CH₃ | CF₃ | Ph-4-Br | CH₂OEt |
| 3-I | 2-CH₃ | CF₃ | Ph-4-Br | CH₂SCH₃ |
| 3-I | 2-CH₃ | CF₃ | Ph-4-Br | CH₂CH═CH₂ |
| 3-I | 2-CH₃ | CF₃ | Ph-4-Br | CH₂CH═CF₂ |
| 3-I | 2-CH₃ | CF₃ | Ph-4-Br | CH₂CH═CCl₂ |
| 3-I | 2-CH₃ | CF₃ | Ph-4-Br | CH₂CF═CF₂ |
| 3-I | 2-CH₃ | CF₃ | Ph-4-Br | CH₂CCl═CCl₂ |
| 3-I | 2-CH₃ | CF₃ | Ph-4-Br | CH₂C≡CH |
| 3-I | 2-CH₃ | CF₃ | Ph-4-Br | CH₂Ph |
| 3-I | 2-CH₃ | CF₃ | Ph-4-Br | C(O)CH₃ |
| 3-I | 2-CH₃ | CF₃ | Ph-4-Br | C(O)Bu-t |
| 3-I | 2-CH₃ | CF₃ | Ph-4-Br | C(O)SCH₃ |
| 3-I | 2-CH₃ | CF₃ | Ph-4-Br | C(S)SEt |
| 3-I | 2-CH₃ | CF₃ | Ph-4-Br | Si(CH₃)₃ |
| 3-I | 2-CH₃ | CF₃ | Ph-4-Br | Si(CH₃)₂Bu-t |
| 3-I | 2-CH₃ | CF₃ | Ph-4-Br | Si(CH₃)₂Ph |
| 3-I | 2-CH₃ | CF₃ | Ph-4-I | CH₃ |
| 3-I | 2-CH₃ | CF₃ | Ph-4-I | CH₂OCH₃ |
| 3-I | 2-CH₃ | CF₃ | Ph-4-I | C(O)CH₃ |
| 3-I | 2-CH₃ | CF₃ | Ph-4-I | Si(CH₃)₃ |
| 3-I | 2-CH₃ | CF₃ | Ph-4-CF₃ | CH₃ |
| 3-I | 2-CH₃ | CF₃ | Ph-4-CF₃ | Et |
| 3-I | 2-CH₃ | CF₃ | Ph-4-CF₃ | n-Pr |
| 3-I | 2-CH₃ | CF₃ | Ph-4-CF₃ | i-Pr |
| 3-I | 2-CH₃ | CF₃ | Ph-4-CF₃ | n-Bu |
| 3-I | 2-CH₃ | CF₃ | Ph-4-CF₃ | CH₂CF₃ |
| 3-I | 2-CH₃ | CF₃ | Ph-4-CF₃ | CH₂OCH₃ |
| 3-I | 2-CH₃ | CF₃ | Ph-4-CF₃ | CH₂OEt |
| 3-I | 2-CH₃ | CF₃ | Ph-4-CF₃ | CH₂SCH₃ |
| 3-I | 2-CH₃ | CF₃ | Ph-4-CF₃ | CH₂CH═CH₂ |
| 3-I | 2-CH₃ | CF₃ | Ph-4-CF₃ | CH₂CH═CF₂ |
| 3-I | 2-CH₃ | CF₃ | Ph-4-CF₃ | CH₂CH═CCl₂ |
| 3-I | 2-CH₃ | CF₃ | Ph-4-CF₃ | CH₂CF═CF₂ |
| 3-I | 2-CH₃ | CF₃ | Ph-4-CF₃ | CH₂CCl═CCl₂ |
| 3-I | 2-CH₃ | CF₃ | Ph-4-CF₃ | CH₂C≡CH |
| 3-I | 2-CH₃ | CF₃ | Ph-4-CF₃ | CH₂Ph |
| 3-I | 2-CH₃ | CF₃ | Ph-4-CF₃ | C(O)CH₃ |
| 3-I | 2-CH₃ | CF₃ | Ph-4-CF₃ | C(O)Bu-t |
| 3-I | 2-CH₃ | CF₃ | Ph-4-CF₃ | C(O)SCH₃ |
| 3-I | 2-CH₃ | CF₃ | Ph-4-CF₃ | C(S)SEt |
| 3-I | 2-CH₃ | CF₃ | Ph-4-CF₃ | Si(CH₃)₃ |
| 3-I | 2-CH₃ | CF₃ | Ph-4-CF₃ | Si(CH₃)₂Bu-t |
| 3-I | 2-CH₃ | CF₃ | Ph-4-CF₃ | Si(CH₃)₂Ph |
| 3-I | 2-CH₃ | CF₃ | Ph-4-CHF₂ | CH₃ |
| 3-I | 2-CH₃ | CF₃ | Ph-4-CHF₂ | CH₂OCH₃ |
| 3-I | 2-CH₃ | CF₃ | Ph-4-CHF₂ | C(O)CH₃ |
| 3-I | 2-CH₃ | CF₃ | Ph-4-CHF₂ | Si(CH₃)₃ |
| 3-I | 2-CH₃ | CF₃ | Ph-4-OCF₃ | CH₃ |
| 3-I | 2-CH₃ | CF₃ | Ph-4-OCF₃ | Et |
| 3-I | 2-CH₃ | CF₃ | Ph-4-OCF₃ | n-Pr |
| 3-I | 2-CH₃ | CF₃ | Ph-4-OCF₃ | i-Pr |
| 3-I | 2-CH₃ | CF₃ | Ph-4-OCF₃ | n-Bu |
| 3-I | 2-CH₃ | CF₃ | Ph-4-OCF₃ | CH₂CF₃ |
| 3-I | 2-CH₃ | CF₃ | Ph-4-OCF₃ | CH₂OCH₃ |
| 3-I | 2-CH₃ | CF₃ | Ph-4-OCF₃ | CH₂OEt |
| 3-I | 2-CH₃ | CF₃ | Ph-4-OCF₃ | CH₂SCH₃ |
| 3-I | 2-CH₃ | CF₃ | Ph-4-OCF₃ | CH₂CH═CH₂ |
| 3-I | 2-CH₃ | CF₃ | Ph-4-OCF₃ | CH₂CH═CF₂ |
| 3-I | 2-CH₃ | CF₃ | Ph-4-OCF₃ | CH₂CH═CCl₂ |
| 3-I | 2-CH₃ | CF₃ | Ph-4-OCF₃ | CH₂CF═CF₂ |
| 3-I | 2-CH₃ | CF₃ | Ph-4-OCF₃ | CH₂CCl═CCl₂ |
| 3-I | 2-CH₃ | CF₃ | Ph-4-OCF₃ | CH₂C≡CH |
| 3-I | 2-CH₃ | CF₃ | Ph-4-OCF₃ | CH₂Ph |
| 3-I | 2-CH₃ | CF₃ | Ph-4-OCF₃ | C(O)CH₃ |
| 3-I | 2-CH₃ | CF₃ | Ph-4-OCF₃ | C(O)Bu-t |
| 3-I | 2-CH₃ | CF₃ | Ph-4-OCF₃ | C(O)SCH₃ |
| 3-I | 2-CH₃ | CF₃ | Ph-4-OCF₃ | C(S)SEt |
| 3-I | 2-CH₃ | CF₃ | Ph-4-OCF₃ | Si(CH₃)₃ |
| 3-I | 2-CH₃ | CF₃ | Ph-4-OCF₃ | Si(CH₃)₂Bu-t |
| 3-I | 2-CH₃ | CF₃ | Ph-4-OCF₃ | Si(CH₃)₂Ph |
| 3-I | 2-CH₃ | CF₃ | Ph-4-OCF₂Br | CH₃ |
| 3-I | 2-CH₃ | CF₃ | Ph-4-OCF₂Br | CH₂OCH₃ |
| 3-I | 2-CH₃ | CF₃ | Ph-4-OCF₂Br | C(O)CH₃ |
| 3-I | 2-CH₃ | CF₃ | Ph-4-OCF₂Br | Si(CH₃)₃ |
| 3-I | 2-CH₃ | CF₃ | Ph-4-OCF₂CHF₂ | CH₃ |
| 3-I | 2-CH₃ | CF₃ | Ph-4-OCF₂CHF₂ | CH₂OCH₃ |
| 3-I | 2-CH₃ | CF₃ | Ph-4-OCF₂CHF₂ | C(O)CH₃ |
| 3-I | 2-CH₃ | CF₃ | Ph-4-OCF₂CHF₂ | Si(CH₃)₃ |
| 3-I | 2-CH₃ | CF₃ | Ph-4-OCF₂CHFCl | CH₃ |
| 3-I | 2-CH₃ | CF₃ | Ph-4-OCF₂CHFCl | CH₂OCH₃ |
| 3-I | 2-CH₃ | CF₃ | Ph-4-OCF₂CHFCl | C(O)CH₃ |
| 3-I | 2-CH₃ | CF₃ | Ph-4-OCF₂CHFCl | Si(CH₃)₃ |
| 3-I | 2-CH₃ | CF₃ | Ph-4-OCF₂CHFCF₃ | CH₃ |
| 3-I | 2-CH₃ | CF₃ | Ph-4-OCF₂CHFCF₃ | CH₂OCH₃ |
| 3-I | 2-CH₃ | CF₃ | Ph-4-OCF₂CHFCF₃ | C(O)CH₃ |
| 3-I | 2-CH₃ | CF₃ | Ph-4-OCF₂CHFCF₃ | Si(CH₃)₃ |
| 3-I | 2-CH₃ | CF₃ | Ph-4-OCF₂CHFOCF₃ | CH₃ |
| 3-I | 2-CH₃ | CF₃ | Ph-4-OCF₂CHFOCF₃ | CH₂OCH₃ |
| 3-I | 2-CH₃ | CF₃ | Ph-4-OCF₂CHFOCF₃ | C(O)CH₃ |
| 3-I | 2-CH₃ | CF₃ | Ph-4-OCF₂CHFOCF₃ | Si(CH₃)₃ |
| 3-I | 2-CH₃ | CF₃ | Ph-4-OCF₂CHFOCF₂CF₂CF₃ | CH₃ |
| 3-I | 2-CH₃ | CF₃ | Ph-4-OCF₂CHFOCF₂CF₂CF₃ | CH₂OCH₃ |
| 3-I | 2-CH₃ | CF₃ | Ph-4-OCF₂CHFOCF₂CF₂CF₃ | C(O)CH₃ |
| 3-I | 2-CH₃ | CF₃ | Ph-4-OCF₂CHFOCF₂CF₂CF₃ | Si(CH₃)₃ |
| 3-I | 2-CH₃ | CF₃ | Ph-4-O(L-45g) | CH₃ |
| 3-I | 2-CH₃ | CF₃ | Ph-4-O(L-45g) | CH₂OCH₃ |
| 3-I | 2-CH₃ | CF₃ | Ph-4-O(L-45g) | C(O)CH₃ |
| 3-I | 2-CH₃ | CF₃ | Ph-4-O(L-45g) | Si(CH₃)₃ |
| 3-I | 2-CH₃ | CF₃ | Ph-3,4-F₂ | CH₃ |
| 3-I | 2-CH₃ | CF₃ | Ph-3,4-F₂ | CH₂OCH₃ |
| 3-I | 2-CH₃ | CF₃ | Ph-3,4-F₂ | C(O)CH₃ |
| 3-I | 2-CH₃ | CF₃ | Ph-3,4-F₂ | Si(CH₃)₃ |
| 3-I | 2-CH₃ | CF₃ | Ph-3-F-4-Cl | CH₃ |
| 3-I | 2-CH₃ | CF₃ | Ph-3-F-4-Cl | CH₂OCH₃ |
| 3-I | 2-CH₃ | CF₃ | Ph-3-F-4-Cl | C(O)CH₃ |
| 3-I | 2-CH₃ | CF₃ | Ph-3-F-4-Cl | Si(CH₃)₃ |
| 3-I | 2-CH₃ | CF₃ | Ph-3,4-Cl₂ | CH₃ |
| 3-I | 2-CH₃ | CF₃ | Ph-3,4-Cl₂ | CH₂OCH₃ |
| 3-I | 2-CH₃ | CF₃ | Ph-3,4-Cl₂ | C(O)CH₃ |
| 3-I | 2-CH₃ | CF₃ | Ph-3,4-Cl₂ | Si(CH₃)₃ |
| 3-I | 2-CH₃ | CF₃ | Ph(-3-OCF₂O-4-) | CH₃ |
| 3-I | 2-CH₃ | CF₃ | Ph(-3-OCF₂O-4-) | CH₂OCH₃ |
| 3-I | 2-CH₃ | CF₃ | Ph(-3-OCF₂O-4-) | C(O)CH₃ |
| 3-I | 2-CH₃ | CF₃ | Ph(-3-OCF₂O-4-) | Si(CH₃)₃ |
| 3-I | 2-CH₃ | CF₃ | Ph(-3-OCF₂CF₂O-4-) | CH₃ |
| 3-I | 2-CH₃ | CF₃ | Ph(-3-OCF₂CF₂O-4-) | CH₂OCH₃ |
| 3-I | 2-CH₃ | CF₃ | Ph(-3-OCF₂CF₂O-4-) | C(O)CH₃ |
| 3-I | 2-CH₃ | CF₃ | Ph(-3-OCF₂CF₂O-4-) | Si(CH₃)₃ |
| 3-I | 2-CH₃ | CF₃ | (L-45c)Cl | CH₃ |
| 3-I | 2-CH₃ | CF₃ | (L-45c)Cl | CH₂OCH₃ |
| 3-I | 2-CH₃ | CF₃ | (L-45c)Cl | C(O)CH₃ |
| 3-I | 2-CH₃ | CF₃ | (L-45c)Cl | Si(CH₃)₃ |
| 3-I | 2-CH₃ | CF₃ | (L-45c)Br | CH₃ |
| 3-I | 2-CH₃ | CF₃ | (L-45c)Br | CH₂OCH₃ |
| 3-I | 2-CH₃ | CF₃ | (L-45c)Br | C(O)CH₃ |
| 3-I | 2-CH₃ | CF₃ | (L-45c)Br | Si(CH₃)₃ |
| 3-I | 2-CH₃ | CF₃ | (L-45c)CF₃ | CH₃ |
| 3-I | 2-CH₃ | CF₃ | (L-45c)CF₃ | Et |
| 3-I | 2-CH₃ | CF₃ | (L-45c)CF₃ | n-Pr |
| 3-I | 2-CH₃ | CF₃ | (L-45c)CF₃ | i-Pr |
| 3-I | 2-CH₃ | CF₃ | (L-45c)CF₃ | n-Bu |
| 3-I | 2-CH₃ | CF₃ | (L-45c)CF₃ | CH₂CF₃ |
| 3-I | 2-CH₃ | CF₃ | (L-45c)CF₃ | CH₂OCH₃ |
| 3-I | 2-CH₃ | CF₃ | (L-45c)CF₃ | CH₂OEt |
| 3-I | 2-CH₃ | CF₃ | (L-45c)CF₃ | CH₂SCH₃ |
| 3-I | 2-CH₃ | CF₃ | (L-45c)CF₃ | CH₂CH═CH₂ |
| 3-I | 2-CH₃ | CF₃ | (L-45c)CF₃ | CH₂CH═CF₂ |
| 3-I | 2-CH₃ | CF₃ | (L-45c)CF₃ | CH₂CH═CCl₂ |
| 3-I | 2-CH₃ | CF₃ | (L-45c)CF₃ | CH₂CF═CF₂ |
| 3-I | 2-CH₃ | CF₃ | (L-45c)CF₃ | CH₂CCl═CCl₂ |
| 3-I | 2-CH₃ | CF₃ | (L-45c)CF₃ | CH₂C≡CH |
| 3-I | 2-CH₃ | CF₃ | (L-45c)CF₃ | CH₂Ph |
| 3-I | 2-CH₃ | CF₃ | (L-45c)CF₃ | C(O)CH₃ |
| 3-I | 2-CH₃ | CF₃ | (L-45c)CF₃ | C(O)Bu-t |
| 3-I | 2-CH₃ | CF₃ | (L-45c)CF₃ | C(O)SCH₃ |
| 3-I | 2-CH₃ | CF₃ | (L-45c)CF₃ | C(S)SEt |
| 3-I | 2-CH₃ | CF₃ | (L-45c)CF₃ | Si(CH₃)₃ |
| 3-I | 2-CH₃ | CF₃ | (L-45c)CF₃ | Si(CH₃)₂Bu-t |
| 3-I | 2-CH₃ | CF₃ | (L-45c)CF₃ | Si(CH₃)₂Ph |
| 3-I | 2-CH₃ | CF₃ | L-45e | CH₃ |
| 3-I | 2-CH₃ | CF₃ | L-45e | CH₂OCH₃ |
| 3-I | 2-CH₃ | CF₃ | L-45e | C(O)CH₃ |
| 3-I | 2-CH₃ | CF₃ | L-45e | Si(CH₃)₃ |
| 3-I | 2-CH₃ | CF₃ | L-45f | CH₃ |
| 3-I | 2-CH₃ | CF₃ | L-45f | CH₂OCH₃ |
| 3-I | 2-CH₃ | CF₃ | L-45f | C(O)CH₃ |
| 3-I | 2-CH₃ | CF₃ | L-45f | Si(CH₃)₃ |
| 3-I | 2-CH₃ | CF₃ | (L-46c)Cl | CH₃ |
| 3-I | 2-CH₃ | CF₃ | (L-46c)Cl | CH₂OCH₃ |
| 3-I | 2-CH₃ | CF₃ | (L-46c)Cl | C(O)CH₃ |
| 3-I | 2-CH₃ | CF₃ | (L-46c)Cl | Si(CH₃)₃ |
| 3-I | 2-CH₃ | CF₃ | (L-46c)Br | CH₃ |
| 3-I | 2-CH₃ | CF₃ | (L-46c)Br | CH₂OCH₃ |
| 3-I | 2-CH₃ | CF₃ | (L-46c)Br | C(O)CH₃ |
| 3-I | 2-CH₃ | CF₃ | (L-46c)Br | Si(CH₃)₃ |
| 3-I | 2-CH₃ | CF₃ | (L-46c)CF₃ | CH₃ |
| 3-I | 2-CH₃ | CF₃ | (L-46c)CF₃ | CH₂OCH₃ |
| 3-I | 2-CH₃ | CF₃ | (L-46c)CF₃ | C(O)CH₃ |
| 3-I | 2-CH₃ | CF₃ | (L-46c)CF₃ | Si(CH₃)₃ |
| 3-I | 2-CH₃ | CF₃ | L-46d | CH₃ |
| 3-I | 2-CH₃ | CF₃ | L-46d | CH₂OCH₃ |
| 3-I | 2-CH₃ | CF₃ | L-46d | C(O)CH₃ |
| 3-I | 2-CH₃ | CF₃ | L-46d | Si(CH₃)₃ |
| 3-I | 2-Pr-n | CF₃ | Ph-4-OCF₃ | H |
| 3-I | 2-Pr-i | CF₃ | (L-45c)CF₃ | H |
| 3-I | 2-Bu-n | CF₃ | Ph-4-Br | H |
| 3-I | 2-CF₃ | CF₃ | Ph-4-CF₃ | H |
| 3-I | 2-CF₂CF₃ | CF₃ | Ph-4-OCF₃ | H |
| 3-I | 2-CH₂OH | CF₃ | Ph-4-Br | H |
| 3-I | 2-CH₂OH | CF₃ | Ph-4-CF₃ | H |
| 3-I | 2-CH₂OH | CF₃ | Ph-4-OCF₃ | H |
| 3-I | 2-CH₂OH | CF₃ | (L-45c)CF₃ | H |
| 3-I | 2-CH₂OCH₃ | CF₃ | (L-45c)CF₃ | H |
| 3-I | 2-CH₂SCH₃ | CF₃ | Ph-4-Br | H |
| 3-I | 2-OCH₃ | CF₃ | Ph-4-Br | H |
| 3-I | 2-OCH₃ | CF₃ | Ph-4-CF₃ | H |
| 3-I | 2-OCH₃ | CF₃ | Ph-4-OCF₃ | H |
| 3-I | 2-OCH₃ | CF₃ | (L-45c)CF₃ | H |
| 3-I | 2-OEt | CF₃ | Ph-4-CF₃ | H |
| 3-I | 2-OPr-i | CF₃ | Ph-4-OCF₃ | H |
| 3-I | 2-OCF₃ | CF₃ | (L-45c)CF₃ | H |
| 3-I | 2-OPh | CF₃ | Ph-4-Br | H |
| 3-I | 2-SCH₃ | CF₃ | Ph-4-Br | H |
| 3-I | 2-SCH₃ | CF₃ | Ph-4-CF₃ | H |
| 3-I | 2-SCH₃ | CF₃ | Ph-4-OCF₃ | H |
| 3-I | 2-SCH₃ | CF₃ | (L-45c)CF₃ | H |
| 3-I | 2-SO₂CH₃ | CF₃ | Ph-4-CF₃ | H |
| 3-I | 2-SEt | CF₃ | Ph-4-OCF₃ | H |
| 3-I | 2-SPr-i | CF₃ | (L-45c)CF₃ | H |
| 3-I | 2-SCHF₂ | CF₃ | Ph-4-Br | H |
| 3-I | 2-N(CH₃)₂ | CF₃ | Ph-4-CF₃ | H |
| 3-I | 2-CN | CF₃ | Ph-4-OCF₃ | H |
| 3-I | 2-Ph | CF₃ | (L-45c)CF₃ | H |
| 3-I | 2-(L-14a) | CF₃ | Ph-4-Br | H |
| 3-I | 2-(L-14b) | CF₃ | Ph-4-CF₃ | H |
| 3-I | 2-(L-14c) | CF₃ | Ph-4-OCF₃ | H |
| 3-I | 2-(L-14d) | CF₃ | (L-45c)CF₃ | H |
| 3-I | 2-(L-14e) | CF₃ | Ph-4-Br | H |
| 3-I | 2-(L-14f) | CF₃ | Ph-4-CF₃ | H |
| 3-I | 2-CH₃-3-F | CF₃ | Ph-4-OCF₃ | H |
| 3-I | 2-CH₃-5-F | CF₃ | (L-45c)CF₃ | H |
| 3-I | 2-CH₃-3-Cl | CF₃ | Ph-4-Br | H |
| 3-I | 2-CH₃-5-Cl | CF₃ | Ph-4-CF₃ | H |
| 3-I | 2,3-(CH₃)₂ | CF₃ | Ph-4-OCF₃ | H |
| 3-I | 2,5-(CH₃)₂ | CF₃ | (L-45c)CF₃ | H |
| 3-I | 2,6-(CH₃)₂ | CF₃ | Ph-4-Br | H |
| 3-I | 2-CH₃-3-OCH₃ | CF₃ | Ph-4-CF₃ | H |
| 3-I | 2-CH₃-3-OCHF₂ | CF₃ | Ph-4-OCF₃ | H |
| 3-I | 2-CH₃-3,5-Cl₂ | CF₃ | (L-45c)CF₃ | H |
| 4-I | 2-CH₃ | CF₃ | Ph-4-Br | H |
| 5-I | 2-CH₃ | CF₃ | Ph-4-CF₃ | H |
| 6-I | 2-CH₃ | CF₃ | Ph-4-OCF₃ | H |
| 3-I-4-Cl | 2-CH₃ | CF₃ | (L-45c)CF₃ | H |
| 4-Cl-6-I | 2-CH₃ | CF₃ | Ph-4-Br | H |
| 5-CH₃ | 2-CH₃ | CF₃ | Ph-4-CF₃ | H |
| 6-CH₃ | 2-CH₃ | CF₃ | Ph-4-OCF₃ | H |
| 3-Cl-4-CH₃ | 2-CH₃ | CF₃ | Ph-4-Br | H |
| 3-Cl-4-CH₃ | 2-CH₃ | CF₃ | Ph-4-CF₃ | H |
| 3-Cl-4-CH₃ | 2-CH₃ | CF₃ | Ph-4-OCF₃ | H |
| 3-Cl-4-CH₃ | 2-CH₃ | CF₃ | (L-45c)CF₃ | H |
| 6-Et | 2-CH₃ | CF₃ | (L-45c)CF₃ | H |
| 4-CF₃ | 2-CH₃ | CF₃ | Ph-4-Br | H |
| 6-CF₃ | 2-CH₃ | CF₃ | Ph-4-CF₃ | H |
| 3-CF₂CF₃ | 2-CH₃ | CF₃ | Ph-4-OCF₃ | H |
| 3-OCH₃ | 2-CH₃ | CF₃ | (L-45c)CF₃ | H |
| 6-OCH₃ | 2-CH₃ | CF₃ | Ph-4-Br | H |
| 3-Cl-4-OCH₃ | 2-CH₃ | CF₃ | Ph-4-CF₃ | H |
| 3-OCH₂O-4 | 2-CH₃ | CF₃ | Ph-4-OCF₃ | H |
| 5-OCHF₂ | 2-CH₃ | CF₃ | (L-45c)CF₃ | H |
| 6-OCHF₂ | 2-CH₃ | CF₃ | Ph-4-Br | H |
| 5-OCF₃ | 2-CH₃ | CF₃ | Ph-4-CF₃ | H |
| 3-OSO₂CH₃ | 2-CH₃ | CF₃ | CF₂O(Ph-4-Cl) | H |
| 3-OSO₂CH₃ | 2-CH₃ | CF₃ | CH═CH(Ph-4-Cl) | H |
| 3-OSO₂CH₃ | 2-CH₃ | CF₃ | CH═CH(Ph-4-Br) | H |
| 3-OSO₂CH₃ | 2-CH₃ | CF₃ | CH═CH(Ph-4-CF₃) | H |
| 3-OSO₂CH₃ | 2-CH₃ | CF₃ | CH═CH(Ph-4-OCF₃) | H |
| 3-OSO₂CH₃ | 2-CH₃ | CF₃ | CH═CH(Ph-3,4-Cl₂) | H |
| 3-OSO₂CH₃ | 2-CH₃ | CF₃ | Ph-4-F | H |
| 3-OSO₂CH₃ | 2-CH₃ | CF₃ | Ph-4-Cl | H |
| 3-OSO₂CH₃ | 2-CH₃ | CF₃ | Ph-4-Br | H |
| 3-OSO₂CH₃ | 2-CH₃ | CF₃ | Ph-4-CF₃ | H |
| 3-OSO₂CH₃ | 2-CH₃ | CF₃ | Ph-4-OCF₃ | H |
| 3-OSO₂CH₃ | 2-CH₃ | CF₃ | Ph-4-OCF₂Br | H |
| 3-OSO₂CH₃ | 2-CH₃ | CF₃ | Ph-4-OCF₂CHF₂ | H |
| 3-OSO₂CH₃ | 2-CH₃ | CF₃ | Ph-4-OCF₂CHFCF₃ | H |
| 3-OSO₂CH₃ | 2-CH₃ | CF₃ | Ph-4-OCF₂CHFOCF₃ | H |
| 3-OSO₂CH₃ | 2-CH₃ | CF₃ | Ph-4-OCF₂CHFOCF₂CF₂CF₃ | H |
| 3-OSO₂CH₃ | 2-CH₃ | CF₃ | Ph-4-O(L-45g) | H |
| 3-OSO₂CH₃ | 2-CH₃ | CF₃ | Ph-3,4-F₂ | H |
| 3-OSO₂CH₃ | 2-CH₃ | CF₃ | Ph-3-F-4-Cl | H |
| 3-OSO₂CH₃ | 2-CH₃ | CF₃ | Ph-3,4-Cl₂ | H |
| 3-OSO₂CH₃ | 2-CH₃ | CF₃ | Ph(-3-OCF₂O-4-) | H |
| 3-OSO₂CH₃ | 2-CH₃ | CF₃ | Ph(-3-OCF₂CF₂O-4-) | H |
| 3-OSO₂CH₃ | 2-CH₃ | CF₃ | (L-45c)CF₃ | H |
| 3-OSO₂CH₃ | 2-CH₃ | CF₃ | L-45f | H |
| 3-OSO₂CH₃ | 2-CH₃ | CF₃ | (L-46c)CF₃ | H |
| 3-OSO₂CH₃ | 2-CH₃ | CF₃ | L-46d | H |
| 3-OSO₂Et | 2-CH₃ | CF₃ | Ph-4-OCF₃ | H |
| 3-OSO₂CF₃ | 2-CH₃ | CF₃ | (L-45c)CF₃ | H |
| 3-OCF₂O-4 | 2-CH₃ | CF₃ | CF₂O(Ph-4-Cl) | H |
| 3-OCF₂O-4 | 2-CH₃ | CF₃ | CH═CH(Ph-4-Cl) | H |
| 3-OCF₂O-4 | 2-CH₃ | CF₃ | CH═CH(Ph-4-Br) | H |
| 3-OCF₂O-4 | 2-CH₃ | CF₃ | CH═CH(Ph-4-CF₃) | H |
| 3-OCF₂O-4 | 2-CH₃ | CF₃ | CH═CH(Ph-4-OCF₃) | H |
| 3-OCF₂O-4 | 2-CH₃ | CF₃ | CH═CH(Ph-3,4-Cl₂) | H |
| 3-OCF₂O-4 | 2-CH₃ | CF₃ | Ph-4-F | H |
| 3-OCF₂O-4 | 2-CH₃ | CF₃ | Ph-4-Cl | H |
| 3-OCF₂O-4 | 2-CH₃ | CF₃ | Ph-4-Br | H |
| 3-OCF₂O-4 | 2-CH₃ | CF₃ | Ph-4-CF₃ | H |
| 3-OCF₂O-4 | 2-CH₃ | CF₃ | Ph-4-OCF₃ | H |
| 3-OCF₂O-4 | 2-CH₃ | CF₃ | Ph-4-OCF₂Br | H |
| 3-OCF₂O-4 | 2-CH₃ | CF₃ | Ph-4-OCF₂CHF₂ | H |
| 3-OCF₂O-4 | 2-CH₃ | CF₃ | Ph-4-OCF₂CHFCF₃ | H |
| 3-OCF₂O-4 | 2-CH₃ | CF₃ | Ph-4-OCF₂CHFOCF₃ | H |
| 3-OCF₂O-4 | 2-CH₃ | CF₃ | Ph-4-OCF₂CHFOCF₂CF₂CF₃ | H |
| 3-OCF₂O-4 | 2-CH₃ | CF₃ | Ph-4-O(L-45g) | H |
| 3-OCF₂O-4 | 2-CH₃ | CF₃ | Ph-3,4-F₂ | H |
| 3-OCF₂O-4 | 2-CH₃ | CF₃ | Ph-3-F-4-Cl | H |
| 3-OCF₂O-4 | 2-CH₃ | CF₃ | Ph-3,4-Cl₂ | H |
| 3-OCF₂O-4 | 2-CH₃ | CF₃ | Ph(-3-OCF₂O-4-) | H |
| 3-OCF₂O-4 | 2-CH₃ | CF₃ | Ph(-3-OCF₂CF₂O-4-) | H |
| 3-OCF₂O-4 | 2-CH₃ | CF₃ | (L-45c)CF₃ | H |
| 3-OCF₂O-4 | 2-CH₃ | CF₃ | L-45f | H |
| 3-OCF₂O-4 | 2-CH₃ | CF₃ | (L-46c)CF₃ | H |
| 3-OCF₂O-4 | 2-CH₃ | CF₃ | L-46d | H |
| 3-OCF₂CF₂O-4 | 2-CH₃ | CF₃ | CH═CH(Ph-4-Cl) | H |
| 3-OCF₂CF₂O-4 | 2-CH₃ | CF₃ | Ph-4-Cl | H |
| 3-OCF₂CF₂O-4 | 2-CH₃ | CF₃ | Ph-4-Br | H |
| 3-OCF₂CF₂O-4 | 2-CH₃ | CF₃ | Ph-4-CF₃ | H |
| 3-OCF₂CF₂O-4 | 2-CH₃ | CF₃ | Ph-4-OCF₃ | H |
| 3-OCF₂CF₂O-4 | 2-CH₃ | CF₃ | Ph-4-OCF₂Br | H |
| 3-OCF₂CF₂O-4 | 2-CH₃ | CF₃ | Ph-4-OCF₂CHFCF₃ | H |
| 3-OCF₂CF₂O-4 | 2-CH₃ | CF₃ | Ph-4-OCF₂CHFOCF₃ | H |
| 3-OCF₂CF₂O-4 | 2-CH₃ | CF₃ | Ph-4-OCF₂CHFOCF₂CF₂CF₃ | H |
| 3-OCF₂CF₂O-4 | 2-CH₃ | CF₃ | Ph-4-O(L-45g) | H |
| 3-OCF₂CF₂O-4 | 2-CH₃ | CF₃ | Ph-3,4-Cl₂ | H |
| 3-OCF₂CF₂O-4 | 2-CH₃ | CF₃ | Ph(-3-OCF₂O-4-) | H |
| 3-OCF₂CF₂O-4 | 2-CH₃ | CF₃ | Ph(-3-OCF₂CF₂O-4-) | H |
| 3-OCF₂CF₂O-4 | 2-CH₃ | CF₃ | (L-45c)CF₃ | H |
| 3-OCF₂CF₂O-4 | 2-CH₃ | CF₃ | L-45f | H |
| 3-OCF₂CF₂O-4 | 2-CH₃ | CF₃ | (L-46c)CF₃ | H |
| 3-OCF₂CF₂O-4 | 2-CH₃ | CF₃ | L-46d | H |
| 6-SCH₃ | 2-CH₃ | CF₃ | Ph-4-Br | H |
| 6-SO₂CH₃ | 2-CH₃ | CF₃ | Ph-4-CF₃ | H |
| 3-SEt | 2-CH₃ | CF₃ | Ph-4-OCF₃ | H |
| 3-SO₂Et | 2-CH₃ | CF₃ | (L-45c)CF₃ | H |
| 3-SPr-i | 2-CH₃ | CF₃ | Ph-4-Br | H |
| 3-S(O)Pr-i | 2-CH₃ | CF₃ | Ph-4-CF₃ | H |
| 3-SO₂Pr-i | 2-CH₃ | CF₃ | Ph-4-OCF₃ | H |
| 6-S(O)CF₃ | 2-CH₃ | CF₃ | (L-45c)CF₃ | H |
| 3-SCH₂CF₃ | 2-CH₃ | CF₃ | Ph-4-Br | H |
| 6-SCH₂CF₃ | 2-CH₃ | CF₃ | Ph-4-CF₃ | H |
| 3-S(O)CH₂CF₃ | 2-CH₃ | CF₃ | Ph-4-OCF₃ | H |
| 6-S(O)CH₂CF₃ | 2-CH₃ | CF₃ | (L-45c)CF₃ | H |
| 3-SO₂CH₂CF₃ | 2-CH₃ | CF₃ | Ph-4-Br | H |
| 3-Cl-6-SCF₃ | 2-CH₃ | CF₃ | Ph-4-CF₃ | H |
| 6-NO₂ | 2-CH₃ | CF₃ | Ph-4-OCF₃ | |
| 3-CN | 2-CH₃ | CF₃ | (L-45c)CF₃ | H |
| 6-C(O)OCH₃ | 2-CH₃ | CF₃ | Ph-4-Br | H |
| 3-C(O)NHPr-i | 2-CH₃ | CF₃ | Ph-4-CF₃ | H |
| 3-C≡CH | 2-CH₃ | CF₃ | Ph-4-OCF₃ | H |
| 3-C≡CSi(CH₃)₃ | 2-CH₃ | CF₃ | (L-45c)CF₃ | H |
| 6-C≡CH | 2-CH₃ | CF₃ | Ph-4-Br | H |
| 6-Ph | 2-CH₃ | CF₃ | Ph-4-CF₃ | H |
| 3-CH═CH—CH═CH-4 | 2-CH₃ | CF₃ | Ph-4-OCF₃ | H |
| 4-CH═CH—CH═CH-5 | 2-CH₃ | CF₃ | (L-15c)CF₃ | H |
| 3-Si(CH₃)₃ | 2-CH₃ | CF₃ | Ph-4-Br | H |

TABLE 4
In the table, the number(s) showing the position(s) of the substituent (X)ₘ and (Y)ₙ correspond to the number(s)
shown in the following structural formulae, and the symbol — means unsubstituted.

| | |
|---|---|
| 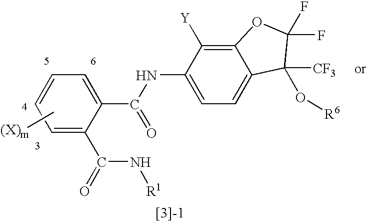 | 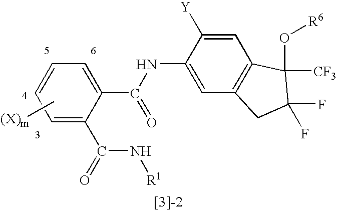 |

| (X)ₘ | Y | R¹ | R⁶ |
|---|---|---|---|
| 3-F | CH₃ | i-Pr | H |
| 3-F | CH₃ | CH(CH₃)CH₂SCH₃ | H |
| 3-F | CH₃ | CH(CH₃)CH₂S(O)CH₃ | H |
| 3-F | CH₃ | CH(CH₃)CH₂SO₂CH₃ | H |
| 3-F | CH₃ | C(CH₃)₂CH₂SCH₃ | H |
| 3-F | CH₃ | C(CH₃)₂CH₂S(O)CH₃ | H |
| 3-F | CH₃ | C(CH₃)₂CH₂SO₂CH₃ | H |
| 3-Cl | CH₃ | i-Pr | H |
| 3-Cl | CH₃ | CH(CH₃)CH₂SCH₃ | H |
| 3-Cl | CH₃ | CH(CH₃)CH₂S(O)CH₃ | H |
| 3-Cl | CH₃ | CH(CH₃)CH₂SO₂CH₃ | H |
| 3-Cl | CH₃ | C(CH₃)₂CH₂SCH₃ | H |
| 3-Cl | CH₃ | C(CH₃)₂CH₂S(O)CH₃ | H |
| 3-Cl | CH₃ | C(CH₃)₂CH₂SO₂CH₃ | H |
| 3,4-Cl₂ | CH₃ | i-Pr | H |
| 3,4-Cl₂ | CH₃ | CH(CH₃)CH₂SCH₃ | H |
| 3,4-Cl₂ | CH₃ | CH(CH₃)CH₂S(O)CH₃ | H |
| 3,4-Cl₂ | CH₃ | CH(CH₃)CH₂SO₂CH₃ | H |
| 3,4-Cl₂ | CH₃ | C(CH₃)₂CH₂SCH₃ | H |
| 3,4-Cl₂ | CH₃ | C(CH₃)₂CH₂S(O)CH₃ | H |
| 3,4-Cl₂ | CH₃ | C(CH₃)₂CH₂SO₂CH₃ | H |
| 3-Br | CH₃ | i-Pr | H |
| 3-Br | CH₃ | CH(CH₃)CH₂SCH₃ | H |
| 3-Br | CH₃ | CH(CH₃)CH₂S(O)CH₃ | H |
| 3-Br | CH₃ | CH(CH₃)CH₂SO₂CH₃ | H |
| 3-Br | CH₃ | C(CH₃)₂CH₂SCH₃ | H |
| 3-Br | CH₃ | C(CH₃)₂CH₂S(O)CH₃ | H |
| 3-Br | CH₃ | C(CH₃)₂CH₂SO₂CH₃ | H |
| 3-I | — | i-Pr | H |
| 3-I | — | CH(CH₃)CH₂SCH₃ | H |
| 3-I | — | CH(CH₃)CH₂S(O)CH₃ | H |
| 3-I | — | CH(CH₃)CH₂SO₂CH₃ | H |
| 3-I | — | C(CH₃)₂CH₂SCH₃ | H |
| 3-I | — | C(CH₃)₂CH₂S(O)CH₃ | H |
| 3-I | — | C(CH₃)₂CH₂SO₂CH₃ | H |
| 3-I | F | i-Pr | H |
| 3-I | F | CH(CH₃)CH₂SCH₃ | H |
| 3-I | F | CH(CH₃)CH₂S(O)CH₃ | H |
| 3-I | F | CH(CH₃)CH₂SO₂CH₃ | H |
| 3-I | F | C(CH₃)₂CH₂SCH₃ | H |
| 3-I | F | C(CH₃)₂CH₂S(O)CH₃ | H |
| 3-I | F | C(CH₃)₂CH₂SO₂CH₃ | H |
| 3-I | Cl | i-Pr | H |
| 3-I | Cl | CH(CH₃)CH₂SCH₃ | H |
| 3-I | Cl | CH(CH₃)CH₂S(O)CH₃ | H |
| 3-I | Cl | CH(CH₃)CH₂SO₂CH₃ | H |
| 3-I | Cl | C(CH₃)₂CH₂SCH₃ | H |
| 3-I | Cl | C(CH₃)₂CH₂S(O)CH₃ | H |
| 3-I | Cl | C(CH₃)₂CH₂SO₂CH₃ | H |
| 3-I | Br | i-Pr | H |
| 3-I | Br | CH(CH₃)CH₂SCH₃ | H |
| 3-I | Br | CH(CH₃)CH₂S(O)CH₃ | H |
| 3-I | Br | CH(CH₃)CH₂SO₂CH₃ | H |
| 3-I | Br | C(CH₃)₂CH₂SCH₃ | H |
| 3-I | Br | C(CH₃)₂CH₂S(O)CH₃ | H |
| 3-I | Br | C(CH₃)₂CH₂SO₂CH₃ | H |
| 3-I | CH₃ | CH₃ | H |
| 3-I | CH₃ | Et | H |
| 3-I | CH₃ | i-Pr | H |
| 3-I | CH₃ | i-Pr | CH₃ |
| 3-I | CH₃ | i-Pr | CH₂OCH₃ |
| 3-I | CH₃ | i-Pr | C(O)CH₃ |
| 3-I | CH₃ | i-Pr | Si(CH₃)₃ |
| 3-I | CH₃ | s-Bu | H |
| 3-I | CH₃ | t-Bu | H |
| 3-I | CH₃ | CH(CH₃)Pr-n | H |
| 3-I | CH₃ | CH(CH₃)CH₂OCH₃ | H |
| 3-I | CH₃ | CH(CH₃)CH₂OC(O)CH₃ | H |
| 3-I | CH₃ | CH(CH₃)CH₂OC(O)NHCH₃ | H |
| 3-I | CH₃ | CH(CH₃)CH₂OC(O)NHEt | H |
| 3-I | CH₃ | CH(CH₃)CH₂OC(O)NHPr-n | H |
| 3-I | CH₃ | CH(CH₃)CH₂OC(O)NHPr-i | H |
| 3-I | CH₃ | CH(CH₃)CH₂OC(O)NHPr-c | H |
| 3-I | CH₃ | CH(CH₃)CH₂OC(O)NHCH₂Ph | H |
| 3-I | CH₃ | CH(CH₃)CH₂OC(O)NHPh | H |
| 3-I | CH₃ | CH(CH₃)CH₂OC(O)N(Et)₂ | H |
| 3-I | CH₃ | CH(CH₃)CH₂OP(S)(OCH₃)₂ | H |
| 3-I | CH₃ | CH(CH₃)CH₂OP(S)(OEt)₂ | H |
| 3-I | CH₃ | C(CH₃)₂CH₂OCH₃ | H |
| 3-I | CH₃ | C(CH₃)₂CH₂OC(O)NHCH₃ | H |
| 3-I | CH₃ | C(CH₃)₂CH₂OC(O)NHEt | H |
| 3-I | CH₃ | C(CH₃)₂CH₂OC(O)NHPr-n | H |
| 3-I | CH₃ | C(CH₃)₂CH₂OC(O)NHCH₂Ph | H |
| 3-I | CH₃ | C(CH₃)₂CH₂OC(O)N(CH₃)₂ | H |
| 3-I | CH₃ | CH(CH₃)CH₂CH₂OEt | H |
| 3-I | CH₃ | CH(CH₃)CH₂SCH₃ | H |
| 3-I | CH₃ | CH(CH₃)CH₂SCH₃ | CH₃ |
| 3-I | CH₃ | CH(CH₃)CH₂SCH₃ | CH₂OCH₃ |
| 3-I | CH₃ | CH(CH₃)CH₂SCH₃ | C(O)CH₃ |
| 3-I | CH₃ | CH(CH₃)CH₂SCH₃ | Si(CH₃)₃ |
| 3-I | CH₃ | CH(CH₃)CH₂S(O)CH₃ | H |
| 3-I | CH₃ | CH(CH₃)CH₂S(O)CH₃ | CH₃ |
| 3-I | CH₃ | CH(CH₃)CH₂S(O)CH₃ | CH₂OCH₃ |
| 3-I | CH₃ | CH(CH₃)CH₂S(O)CH₃ | C(O)CH₃ |
| 3-I | CH₃ | CH(CH₃)CH₂S(O)CH₃ | Si(CH₃)₃ |
| 3-I | CH₃ | CH(CH₃)CH₂SO₂CH₃ | H |
| 3-I | CH₃ | CH(CH₃)CH₂SO₂CH₃ | CH₃ |
| 3-I | CH₃ | CH(CH₃)CH₂SO₂CH₃ | CH₂OCH₃ |
| 3-I | CH₃ | CH(CH₃)CH₂SO₂CH₃ | C(O)CH₃ |
| 3-I | CH₃ | CH(CH₃)CH₂SO₂CH₃ | Si(CH₃)₃ |
| 3-I | CH₃ | CH(CH₃)CH₂SEt | H |
| 3-I | CH₃ | CH(CH₃)CH₂S(O)Et | H |
| 3-I | CH₃ | CH(CH₃)CH₂SO₂Et | H |
| 3-I | CH₃ | CH(CH₃)CH₂SCH₂Si(CH₃)₃ | H |
| 3-I | CH₃ | CH(CH₃)CH₂SSCH₃ | H |
| 3-I | CH₃ | CH(CH₃)CH₂SO₂NHEt | H |
| 3-I | CH₃ | CH(CH₃)CH₂SO₂N(Et)₂ | H |
| 3-I | CH₃ | C(CH₃)₂CH₂SCH₃ | H |
| 3-I | CH₃ | C(CH₃)₂CH₂SCH₃ | CH₃ |
| 3-I | CH₃ | C(CH₃)₂CH₂SCH₃ | CH₂OCH₃ |
| 3-I | CH₃ | C(CH₃)₂CH₂SCH₃ | C(O)CH₃ |
| 3-I | CH₃ | C(CH₃)₂CH₂SCH₃ | Si(CH₃)₃ |
| 3-I | CH₃ | C(CH₃)₂CH₂S(O)CH₃ | H |
| 3-I | CH₃ | C(CH₃)₂CH₂S(O)CH₃ | CH₃ |
| 3-I | CH₃ | C(CH₃)₂CH₂S(O)CH₃ | CH₂OCH₃ |
| 3-I | CH₃ | C(CH₃)₂CH₂S(O)CH₃ | C(O)CH₃ |
| 3-I | CH₃ | C(CH₃)₂CH₂S(O)CH₃ | Si(CH₃)₃ |
| 3-I | CH₃ | C(CH₃)₂CH₂SO₂CH₃ | H |
| 3-I | CH₃ | C(CH₃)₂CH₂SO₂CH₃ | CH₃ |
| 3-I | CH₃ | C(CH₃)₂CH₂SO₂CH₃ | CH₂OCH₃ |
| 3-I | CH₃ | C(CH₃)₂CH₂SO₂CH₃ | C(O)CH₃ |
| 3-I | CH₃ | C(CH₃)₂CH₂SO₂CH₃ | Si(CH₃)₃ |
| 3-I | CH₃ | C(CH₃)₂CH₂SEt | H |
| 3-I | CH₃ | C(CH₃)₂CH₂S(O)Et | H |
| 3-I | CH₃ | C(CH₃)₂CH₂SO₂Et | H |
| 3-I | CH₃ | C(CH₃)₂CH₂SO₂N(Et)₂ | H |
| 3-I | CH₃ | CH(CH₃)CH₂N(CH₃)SO₂CH₃ | H |
| 3-I | CH₃ | C(CH₃)₂CH₂NHC(O)OCH₃ | H |
| 3-I | CH₃ | CH(CH₃)CH═NOCH₃ | H |
| 3-I | CH₃ | C(CH₃)₂CH═NOCH₃ | H |
| 3-I | CH₃ | C(CH₃)₂CH═CH₂ | H |
| 3-I | CH₃ | C(CH₃)₂C≡CH | H |
| 3-I | Et | i-Pr | H |
| 3-I | Et | CH(CH₃)CH₂SCH₃ | H |
| 3-I | Et | CH(CH₃)CH₂S(O)CH₃ | H |
| 3-I | Et | CH(CH₃)CH₂SO₂CH₃ | H |
| 3-I | Et | C(CH₃)₂CH₂SCH₃ | H |
| 3-I | Et | C(CH₃)₂CH₂S(O)CH₃ | H |
| 3-I | Et | C(CH₃)₂CH₂SO₂CH₃ | H |
| 3-I | OCH₃ | i-Pr | H |
| 3-I | OCH₃ | CH(CH₃)CH₂SCH₃ | H |
| 3-I | OCH₃ | CH(CH₃)CH₂S(O)CH₃ | H |
| 3-I | OCH₃ | CH(CH₃)CH₂SO₂CH₃ | H |
| 3-I | OCH₃ | C(CH₃)₂CH₂SCH₃ | H |
| 3-I | OCH₃ | C(CH₃)₂CH₂S(O)CH₃ | H |
| 3-I | OCH₃ | C(CH₃)₂CH₂SO₂CH₃ | H |
| 3-I | SCH₃ | i-Pr | H |
| 3-I | SCH₃ | CH(CH₃)CH₂SCH₃ | H |
| 3-I | SCH₃ | CH(CH₃)CH₂S(O)CH₃ | H |
| 3-I | SCH₃ | CH(CH₃)CH₂SO₂CH₃ | H |
| 3-I | SCH₃ | C(CH₃)₂CH₂SCH₃ | H |
| 3-I | SCH₃ | C(CH₃)₂CH₂S(O)CH₃ | H |
| 3-I | SCH₃ | C(CH₃)₂CH₂SO₂CH₃ | H |
| 3-CH₃ | CH₃ | i-Pr | H |
| 3-CH₃ | CH₃ | CH(CH₃)CH₂SCH₃ | H |
| 3-CH₃ | CH₃ | CH(CH₃)CH₂S(O)CH₃ | H |
| 3-CH₃ | CH₃ | CH(CH₃)CH₂SO₂CH₃ | H |
| 3-CH₃ | CH₃ | C(CH₃)₂CH₂SCH₃ | H |
| 3-CH₃ | CH₃ | C(CH₃)₂CH₂S(O)CH₃ | H |
| 3-CH₃ | CH₃ | C(CH₃)₂CH₂SO₂CH₃ | H |
| 3-CF₃ | CH₃ | i-Pr | H |
| 3-CF₃ | CH₃ | CH(CH₃)CH₂SCH₃ | H |
| 3-CF₃ | CH₃ | CH(CH₃)CH₂S(O)CH₃ | H |
| 3-CF₃ | CH₃ | CH(CH₃)CH₂SO₂CH₃ | H |
| 3-CF₃ | CH₃ | C(CH₃)₂CH₂SCH₃ | H |
| 3-CF₃ | CH₃ | C(CH₃)₂CH₂S(O)CH₃ | H |
| 3-CF₃ | CH₃ | C(CH₃)₂CH₂SO₂CH₃ | H |
| 3-OCHF₂ | CH₃ | i-Pr | H |
| 3-OCHF₂ | CH₃ | CH(CH₃)CH₂SCH₃ | H |
| 3-OCHF₂ | CH₃ | CH(CH₃)CH₂S(O)CH₃ | H |
| 3-OCHF₂ | CH₃ | CH(CH₃)CH₂SO₂CH₃ | H |
| 3-OCHF₂ | CH₃ | C(CH₃)₂CH₂SCH₃ | H |
| 3-OCHF₂ | CH₃ | C(CH₃)₂CH₃S(O)CH₃ | H |
| 3-OCHF₂ | CH₃ | C(CH₃)₂CH₂SO₂CH₃ | H |
| 3-OCF₃ | CH₃ | i-Pr | H |
| 3-OCF₃ | CH₃ | CH(CH₃)CH₂SCH₃ | H |
| 3-OCF₃ | CH₃ | CH(CH₃)CH₂S(O)CH₃ | H |
| 3-OCF₃ | CH₃ | CH(CH₃)CH₂SO₂CH₃ | H |
| 3-OCF₃ | CH₃ | C(CH₃)₂CH₂SCH₃ | H |
| 3-OCF₃ | CH₃ | C(CH₃)₂CH₂S(O)CH₃ | H |
| 3-OCF₃ | CH₃ | C(CH₃)₂CH₂SO₂CH₃ | H |
| 3-OSO₂CH₃ | CH₃ | i-Pr | H |
| 3-OSO₂CH₃ | CH₃ | CH(CH₃)CH₂SCH₃ | H |
| 3-OSO₂CH₃ | CH₃ | CH(CH₃)CH₂S(O)CH₃ | H |
| 3-OSO₂CH₃ | CH₃ | CH(CH₃)CH₂SO₂CH₃ | H |
| 3-OSO₂CH₃ | CH₃ | C(CH₃)₂CH₂SCH₃ | H |
| 3-OSO₂CH₃ | CH₃ | C(CH₃)₂CH₂S(O)CH₃ | H |
| 3-OSO₂CH₃ | CH₃ | C(CH₃)₂CH₂SO₂CH₃ | H |
| 3-OCF₂O-4 | CH₃ | i-Pr | H |
| 3-OCF₂O-4 | CH₃ | CH(CH₃)CH₂SCH₃ | H |
| 3-OCF₂O-4 | CH₃ | CH(CH₃)CH₂S(O)CH₃ | H |
| 3-OCF₂O-4 | CH₃ | CH(CH₃)CH₂SO₂CH₃ | H |
| 3-OCF₂O-4 | CH₃ | C(CH₃)₂CH₂SCH₃ | H |
| 3-OCF₂O-4 | CH₃ | C(CH₃)₂CH₂S(O)CH₃ | H |
| 3-OCF₂O-4 | CH₃ | C(CH₃)₂CH₂SO₂CH₃ | H |
| 3-SCH₃ | CH₃ | i-Pr | H |
| 3-SCH₃ | CH₃ | CH(CH₃)CH₂SCH₃ | H |
| 3-SCH₃ | CH₃ | CH(CH₃)CH₂S(O)CH₃ | H |
| 3-SCH₃ | CH₃ | CH(CH₃)CH₂SO₂CH₃ | H |
| 3-SCH₃ | CH₃ | C(CH₃)₂CH₂SCH₃ | H |
| 3-SCH₃ | CH₃ | C(CH₃)₂CH₂S(O)CH₃ | H |
| 3-SCH₃ | CH₃ | C(CH₃)₂CH₂SO₂CH₃ | H |
| 3-S(O)CH₃ | CH₃ | i-Pr | H |
| 3-S(O)CH₃ | CH₃ | CH(CH₃)CH₂SCH₃ | H |
| 3-S(O)CH₃ | CH₃ | CH(CH₃)CH₂S(O)CH₃ | H |
| 3-S(O)CH₃ | CH₃ | CH(CH₃)CH₂SO₂CH₃ | H |
| 3-S(O)CH₃ | CH₃ | C(CH₃)₂CH₂SCH₃ | H |
| 3-S(O)CH₃ | CH₃ | C(CH₃)₂CH₂S(O)CH₃ | H |
| 3-S(O)CH₃ | CH₃ | C(CH₃)₂CH₂SO₂CH₃ | H |
| 3-SO₂CH₃ | CH₃ | i-Pr | H |
| 3-SO₂CH₃ | CH₃ | CH(CH₃)CH₂SCH₃ | H |
| 3-SO₂CH₃ | CH₃ | CH(CH₃)CH₂S(O)CH₃ | H |
| 3-SO₂CH₃ | CH₃ | CH(CH₃)CH₂SO₂CH₃ | H |
| 3-SO₂CH₃ | CH₃ | C(CH₃)₂CH₂SCH₃ | H |
| 3-SO₂CH₃ | CH₃ | C(CH₃)₂CH₂S(O)CH₃ | H |
| 3-SO₂CH₃ | CH₃ | C(CH₃)₂CH₂SO₂CH₃ | H |
| 3-SCHF₂ | CH₃ | i-Pr | H |
| 3-SCHF₂ | CH₃ | CH(CH₃)CH₂SCH₃ | H |
| 3-SCHF₂ | CH₃ | CH(CH₃)CH₂S(O)CH₃ | H |
| 3-SCHF₂ | CH₃ | CH(CH₃)CH₂SO₂CH₃ | H |
| 3-SCHF₂ | CH₃ | C(CH₃)₂CH₂SCH₃ | H |
| 3-SCHF₂ | CH₃ | C(CH₃)₂CH₂S(O)CH₃ | H |
| 3-SCHF₂ | CH₃ | C(CH₃)₂CH₂SO₂CH₃ | H |
| 3-S(O)CHF₂ | CH₃ | i-Pr | H |
| 3-S(O)CHF₂ | CH₃ | CH(CH₃)CH₂SCH₃ | H |
| 3-S(O)CHF₂ | CH₃ | CH(CH₃)CH₂S(O)CH₃ | H |
| 3-S(O)CHF₂ | CH₃ | CH(CH₃)CH₂SO₂CH₃ | H |
| 3-S(O)CHF₂ | CH₃ | C(CH₃)₂CH₂SCH₃ | H |
| 3-S(O)CHF₂ | CH₃ | C(CH₃)₂CH₂S(O)CH₃ | H |
| 3-S(O)CHF₂ | CH₃ | C(CH₃)₂CH₂SO₂CH₃ | H |
| 3-SO₂CHF₂ | CH₃ | i-Pr | H |
| 3-SO₂CHF₂ | CH₃ | CH(CH₃)CH₂SCH₃ | H |
| 3-SO₂CHF₂ | CH₃ | CH(CH₃)CH₂S(O)CH₃ | H |
| 3-SO₂CHF₂ | CH₃ | CH(CH₃)CH₂SO₂CH₃ | H |
| 3-SO₂CHF₂ | CH₃ | C(CH₃)₂CH₂SCH₃ | H |
| 3-SO₂CHF₂ | CH₃ | C(CH₃)₂CH₂S(O)CH₃ | H |
| 3-SO₂CHF₂ | CH₃ | C(CH₃)₂CH₂SO₂CH₃ | H |
| 3-SCF₃ | CH₃ | i-Pr | H |
| 3-SCF₃ | CH₃ | CH(CH₃)CH₂SCH₃ | H |
| 3-SCF₃ | CH₃ | CH(CH₃)CH₂S(O)CH₃ | H |
| 3-SCF₃ | CH₃ | CH(CH₃)CH₂SO₂CH₃ | H |
| 3-SCF₃ | CH₃ | C(CH₃)₂CH₂SCH₃ | H |
| 3-SCF₃ | CH₃ | C(CH₃)₂CH₂S(O)CH₃ | H |
| 3-SCF₃ | CH₃ | C(CH₃)₂CH₂SO₂CH₃ | H |
| 3-S(O)CF₃ | CH₃ | i-Pr | H |
| 3-S(O)CF₃ | CH₃ | CH(CH₃)CH₂SCH₃ | H |
| 3-S(O)CF₃ | CH₃ | CH(CH₃)CH₂S(O)CH₃ | H |
| 3-S(O)CF₃ | CH₃ | CH(CH₃)CH₂SO₂CH₃ | H |
| 3-S(O)CF₃ | CH₃ | C(CH₃)₂CH₃SCH₃ | H |
| 3-S(O)CF₃ | CH₃ | C(CH₃)₂CH₂S(O)CH₃ | H |
| 3-S(O)CF₃ | CH₃ | C(CH₃)₂CH₂SO₂CH₃ | H |
| 3-SO₂CF₃ | CH₃ | i-Pr | H |
| 3-SO₂CF₃ | CH₃ | CH(CH₃)CH₂SCH₃ | H |
| 3-SO₂CF₃ | CH₃ | CH(CH₃)CH₂S(O)CH₃ | H |
| 3-SO₂CF₃ | CH₃ | CH(CH₃)CH₂SO₂CH₃ | H |
| 3-SO₂CF₃ | CH₃ | C(CH₃)₂CH₂SCH₃ | H |
| 3-SO₂CF₃ | CH₃ | C(CH₃)₂CH₂S(O)CH₃ | H |
| 3-SO₂CF₃ | CH₃ | C(CH₃)₂CH₂SO₂CH₃ | H |
| 3-NO₂ | CH₃ | i-Pr | H |
| 3-NO₂ | CH₃ | CH(CH₃)CH₂SCH₃ | H |
| 3-NO₂ | CH₃ | CH(CH₃)CH₂S(O)CH₃ | H |
| 3-NO₂ | CH₃ | CH(CH₃)CH₂SO₂CH₃ | H |
| 3-NO₂ | CH₃ | C(CH₃)₂CH₂SCH₃ | H |
| 3-NO₂ | CH₃ | C(CH₃)₂CH₂S(O)CH₃ | H |
| 3-NO₂ | CH₃ | C(CH₃)₂CH₂SO₂CH₃ | H |

TABLE 5

| | |
|---|---|
| 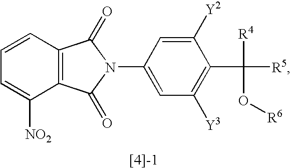 | 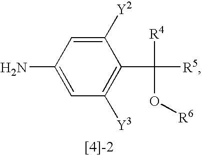 |
| 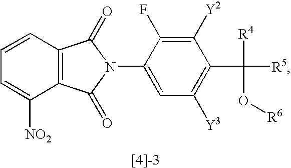 | 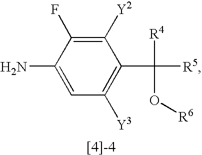 |
| 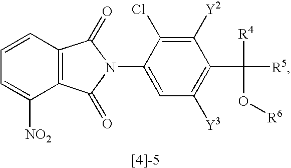 | 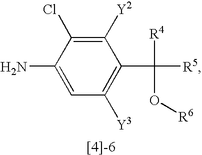 |
| 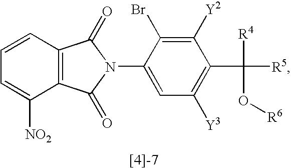 | 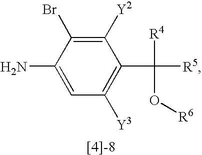 |
| 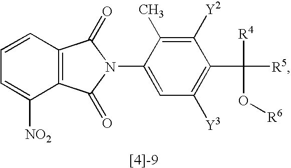 | 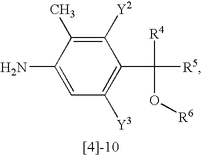 |
| 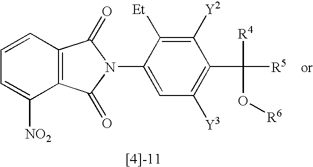 | 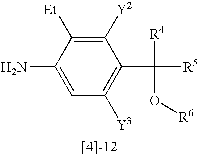 |

| R⁴ | Y² | R⁵ | Y³ | R⁶ |
|---|---|---|---|---|
| CH₃ | H | CF₂O(Ph-4-Cl) | H | H |
| CH₃ | H | CH═CH(Ph-4-Cl) | H | H |
| CH₃ | H | CH═CH(Ph-4-OCF₃) | H | H |
| CH₃ | H | CH═CH(Ph-3,4-Cl₂) | H | H |
| CH₃ | H | Ph-4-F | H | H |
| CH₃ | H | Ph-4-Cl | H | H |
| CH₃ | H | Ph-4-Br | H | H |
| CH₃ | H | Ph-4-I | H | H |
| CH₃ | H | Ph-4-CF₃ | H | H |
| CH₃ | H | Ph-4-OCHF₂ | H | H |
| CH₃ | H | Ph-4-OCF₃ | H | H |
| CH₃ | H | Ph-4-OCF₂Br | H | H |
| CH₃ | H | Ph-4-OCF₂CHF₂ | H | H |
| CH₃ | H | Ph-4-OCF₂CHFCl | H | H |
| CH₃ | H | Ph-4-OCF₂CHFCF₃ | H | H |
| CH₃ | H | Ph-4-OCF₂CHFOCF₃ | H | H |
| CH₃ | H | Ph-4-OCF₂CHFOCF₂CF₂CF₃ | H | H |
| CH₃ | H | Ph-4-O(L-45g) | H | H |
| CH₃ | H | Ph-3,4-Cl₂ | H | H |
| CH₃ | H | Ph(-3-OCF₂O-4-) | H | H |
| CH₃ | H | Ph(-3-OCF₂CF₂O-4-) | H | H |
| CH₃ | H | (L-45c)Cl | H | H |
| CH₃ | H | (L-45c)Br | H | H |
| CH₃ | H | (L-45c)CF₃ | H | H |
| CH₃ | H | L-45e | H | H |
| CH₃ | H | L-45f | H | H |
| CH₃ | H | (L-46c)Cl | H | H |
| CH₃ | H | (L-46c)Br | H | H |
| CH₃ | H | (L-46c)CF₃ | H | H |
| CH₃ | H | L-46d | H | H |
| Et | H | CF₂O(Ph-4-Cl) | H | H |
| Et | H | CH═CH(Ph-4-Cl) | H | H |
| Et | H | CH═CH(Ph-4-OCF₃) | H | H |
| Et | H | CH═CH(Ph-3,4-Cl₂) | H | H |
| Et | H | Ph-4-Cl | H | H |
| Et | H | Ph-4-Br | H | H |
| Et | H | Ph-4-CF₃ | H | H |
| Et | H | Ph-4-OCF₃ | H | H |
| Et | H | Ph-4-OCF₂Br | H | H |
| Et | H | Ph-4-OCF₂CHFCF₃ | H | H |
| Et | H | Ph-4-OCF₂CHFOCF₃ | H | H |
| Et | H | Ph-4-OCF₂CHFOCF₂CF₂CF₃ | H | H |
| Et | H | Ph-4-O(L-45g) | H | H |
| Et | H | Ph-3,4-Cl₂ | H | H |
| Et | H | Ph(-3-OCF₂O-4-) | H | H |
| Et | H | Ph(-3-OCF₂CF₂O-4-) | H | H |
| Et | H | (L-45c)Br | H | H |
| Et | H | (L-45c)CF₃ | H | H |
| Et | H | L-45e | H | H |
| Et | H | L-45f | H | H |
| Et | H | (L-46c)Br | H | H |
| Et | H | (L-46c)CF₃ | H | H |
| Et | H | L-46d | H | H |
| n-Pr | H | Ph-4-Br | H | H |
| n-Pr | H | Ph-4-CF₃ | H | H |
| n-Pr | H | Ph-4-OCF₃ | H | H |
| n-Pr | H | (L-45c)CF₃ | H | H |
| i-Pr | H | Ph-4-Br | H | H |
| i-Pr | H | Ph-4-CF₃ | H | H |
| i-Pr | H | Ph-4-OCF₃ | H | H |
| i-Pr | H | (L-45c)CF₃ | H | H |
| c-Pr | H | Ph-4-Br | H | H |
| c-Pr | H | Ph-4-CF₃ | H | H |
| c-Pr | H | Ph-4-OCF₃ | H | H |
| c-Pr | H | (L-45c)CF₃ | H | H |
| n-Bu | H | Ph-4-Br | H | H |
| CH₂F | H | Ph-4-CF₃ | H | H |
| CH₂Cl | H | Ph-4-OCF₃ | H | H |
| CH₂Br | H | (L-45c)CF₃ | H | H |
| CHF₂ | H | CF₂O(Ph-4-Cl) | H | H |
| CHF₂ | H | CH═CH(Ph-4-Cl) | H | H |
| CHF₂ | H | CH═CH(Ph-4-OCF₃) | H | H |
| CHF₂ | H | CH═CH(Ph-3,4-Cl₂) | H | H |
| CHF₂ | H | Ph-4-F | H | H |
| CHF₂ | H | Ph-4-Cl | H | H |
| CHF₂ | H | Ph-4-Br | H | H |
| CHF₂ | H | Ph-4-I | H | H |
| CHF₂ | H | Ph-4-CF₃ | H | H |
| CHF₂ | H | Ph-4-OCHF₂ | H | H |
| CHF₂ | H | Ph-4-OCF₃ | H | H |
| CHF₂ | H | Ph-4-OCF₂Br | H | H |
| CHF₂ | H | Ph-4-OCF₂CHF₂ | H | H |
| CHF₂ | H | Ph-4-OCF₂CHFCl | H | H |
| CHF₂ | H | Ph-4-OCF₂CHFCF₃ | H | H |
| CHF₂ | H | Ph-4-OCF₂CHFOCF₃ | H | H |
| CHF₂ | H | Ph-4-OCF₂CHFOCF₂CF₂CF₃ | H | H |
| CHF₂ | H | Ph-4-O(L-45g) | H | H |
| CHF₂ | H | Ph-3,4-Cl₂ | H | H |
| CHF₂ | H | Ph(-3-OCF₂O-4-) | H | H |
| CHF₂ | H | Ph(-3-OCF₂CF₂O-4-) | H | H |
| CHF₂ | H | (L-45c)Cl | H | H |
| CHF₂ | H | (L-45c)Br | H | H |
| CHF₂ | H | (L-45c)CF₃ | H | H |
| CHF₂ | H | L-45e | H | H |
| CHF₂ | H | L-45f | H | H |
| CHF₂ | H | (L-46c)Cl | H | H |
| CHF₂ | H | (L-46c)Br | H | H |
| CHF₂ | H | (L-46c)CF₃ | H | H |
| CHF₂ | H | L-46d | H | H |
| CHFCl | H | Ph-4-Br | H | H |
| CHFBr | H | Ph-4-CF₃ | H | H |
| CF₃ | H | CH₂OCH₃ | H | H |
| CF₃ | H | CH₂OEt | H | H |
| CF₃ | H | CH₂OPr-n | H | H |
| CF₃ | H | CH₂OPr-i | H | H |
| CF₃ | H | CH₂OBu-n | H | H |
| CF₃ | H | CH₂OCH₂CF₃ | H | H |
| CF₃ | H | CH₂OCH₂CF₂CF₃ | H | H |
| CF₃ | H | CH₂OCH(CF₃)₂ | H | H |
| CF₃ | H | CH₂OPh | H | H |
| CF₃ | H | CH₂O(Ph-4-F) | H | H |
| CF₃ | H | CH₂O(Ph-2-Cl) | H | H |
| CF₃ | H | CH₂O(Ph-3-Cl) | H | H |
| CF₃ | H | CH₂O(Ph-4-Cl) | H | H |
| CF₃ | H | CH₂O(Ph-4-Br) | H | H |
| CF₃ | H | CH₂O(Ph-4-CF₃) | H | H |
| CF₃ | H | CH₂O(Ph-2-OCF₃) | H | H |
| CF₃ | H | CH₂O(Ph-3-OCF₃) | H | H |
| CF₃ | H | CH₂O(Ph-4-OCF₃) | H | H |
| CF₃ | H | CF₂OPh | H | H |
| CF₃ | H | CF₂O(Ph-3-F) | H | H |
| CF₃ | H | CF₂O(Ph-4-F) | H | H |
| CF₃ | H | CF₂O(Ph-2-Cl) | H | H |
| CF₃ | H | CF₂O(Ph-3-Cl) | H | H |
| CF₃ | H | CF₂O(Ph-4-Cl) | H | H |
| CF₃ | H | CF₂O(Ph-3-Br) | H | H |
| CF₃ | H | CF₂O(Ph-4-Br) | H | H |
| CF₃ | H | CF₂O(Ph-3-CF₃) | H | H |
| CF₃ | H | CF₂O(Ph-4-CF₃) | H | H |
| CF₃ | H | CF₂O(Ph-3-OCF₃) | H | H |
| CF₃ | H | CF₂O(Ph-4-OCF₃) | H | H |
| CF₃ | H | CH₂SPh | H | H |
| CF₃ | H | CH₂S(Ph-3-Cl) | H | H |
| CF₃ | H | CH₂S(Ph-4-Cl) | H | H |
| CF₃ | H | CH₂SO₂(Ph-3-Cl) | H | H |
| CF₃ | H | CH₂SO₂(Ph-4-Cl) | H | H |
| CF₃ | H | CH₂N(CH₃)₂ | H | H |
| CF₃ | H | CH₂NHPh | H | H |
| CF₃ | H | CH₂NH(Ph-3-Cl) | H | H |
| CF₃ | H | CH₂NH(Ph-4-Cl) | H | H |
| CF₃ | H | CF₂C(O)OEt | H | H |
| CF₃ | H | CH₂(L-5a) | H | H |
| CF₃ | H | CH₂(L-14a) | H | H |
| CF₃ | H | CH₂(L-24a) | H | H |
| CF₃ | H | CH₂(L-36a) | H | H |
| CF₃ | H | C(O)OEt | H | H |
| CF₃ | H | C(O)OBu-t | H | H |
| CF₃ | H | C(O)OCH₂CF₃ | H | H |
| CF₃ | H | CH═CHPh | H | H |
| CF₃ | H | CH═CH(Ph-3-F) | H | H |
| CF₃ | H | CH═CH(Ph-4-F) | H | H |
| CF₃ | H | CH═CH(Ph-2-Cl) | H | H |
| CF₃ | H | CH═CH(Ph-3-Cl) | H | H |
| CF₃ | H | CH═CH(Ph-4-Cl) | H | H |
| CF₃ | H | CH═CH(Ph-3-Br) | H | H |
| CF₃ | H | CH═CH(Ph-4-Br) | H | H |
| CF₃ | H | CH═CH(Ph-3-CF₃) | H | H |
| CF₃ | H | CH═CH(Ph-4-CF₃) | H | H |
| CF₃ | H | CH═CH(Ph-3-OCF₃) | H | H |
| CF₃ | H | CH═CH(Ph-4-OCF₃) | H | H |
| CF₃ | H | CH═CH(Ph-3-SCH₃) | H | H |
| CF₃ | H | CH═CH(Ph-4-SCH₃) | H | H |
| CF₃ | H | CH═CH(Ph-3-SO₂CH₃) | H | H |
| CF₃ | H | CH═CH(Ph-4-SO₂CH₃) | H | H |
| CF₃ | H | CH═CH(Ph-3,4-F₂) | H | H |
| CF₃ | H | CH═CH(Ph-3-F-4-Cl) | H | H |
| CF₃ | H | CH═CH(Ph-3,4-Cl₂) | H | H |
| CF₃ | H | CH═CH(Ph-3,4-Br₂) | H | H |
| CF₃ | H | CH═CH(Ph-3-F-4-CF₃) | H | H |
| CF₃ | H | CH═CH(Ph-3-Cl-4-OCF₃) | H | H |
| CF₃ | H | CH═CH [Ph(-3-OCF₂O-4-)] | H | H |
| CF₃ | H | Ph | H | H |
| CF₃ | H | Ph-2-F | H | H |
| CF₃ | H | Ph-3-F | H | H |
| CF₃ | H | Ph-4-F | H | H |
| CF₃ | H | Ph-2-Cl | H | H |
| CF₃ | H | Ph-3-Cl | H | H |
| CF₃ | H | Ph-4-Cl | H | H |
| CF₃ | H | Ph-3-Br | H | H |
| CF₃ | H | Ph-4-Br | H | H |
| CF₃ | H | Ph-4-Br | H | CH₃ |
| CF₃ | H | Ph-4-Br | H | Et |
| CF₃ | H | Ph-4-Br | H | n-Pr |
| CF₃ | H | Ph-4-Br | H | i-Pr |
| CF₃ | H | Ph-4-Br | H | n-Bu |
| CF₃ | H | Ph-4-Br | H | CH₂CF₃ |
| CF₃ | H | Ph-4-Br | H | CH₂OCH₃ |
| CF₃ | H | Ph-4-Br | H | CH₂OEt |
| CF₃ | H | Ph-4-Br | H | CH₂SCH₃ |
| CF₃ | H | Ph-4-Br | H | CH₂CH═CH₂ |
| CF₃ | H | Ph-4-Br | H | CH₂CH═CF₂ |
| CF₃ | H | Ph-4-Br | H | CH₂CH═CCl₂ |
| CF₃ | H | Ph-4-Br | H | CH₂CF═CF₂ |
| CF₃ | H | Ph-4-Br | H | CH₂CCl═CCl₂ |
| CF₃ | H | Ph-4-Br | H | CH₂C≡CH |
| CF₃ | H | Ph-4-Br | H | CH₂Ph |
| CF₃ | H | Ph-4-Br | H | Si(CH₃) |
| CF₃ | H | Ph-4-Br | H | Si(CH₃)₂Bu-t |
| CF₃ | H | Ph-4-Br | H | Si(CH₃)₂Ph |
| CF₃ | H | Ph-4-I | H | H |
| CF₃ | H | Ph-4-Bu-t | H | H |
| CF₃ | H | Ph-3-CF₃ | H | H |
| CF₃ | H | Ph-4-CF₃ | H | H |
| CF₃ | H | Ph-4-CF₃ | H | CH₃ |
| CF₃ | H | Ph-4-CF₃ | H | Et |
| CF₃ | H | Ph-4-CF₃ | H | n-Pr |
| CF₃ | H | Ph-4-CF₃ | H | i-Pr |
| CF₃ | H | Ph-4-CF₃ | H | n-Bu |
| CF₃ | H | Ph-4-CF₃ | H | CH₂CF₃ |
| CF₃ | H | Ph-4-CF₃ | H | CH₂OCH₃ |
| CF₃ | H | Ph-4-CF₃ | H | CH₂OEt |
| CF₃ | H | Ph-4-CF₃ | H | CH₂SCH₃ |
| CF₃ | H | Ph-4-CF₃ | H | CH₂CH═CH₂ |
| CF₃ | H | Ph-4-CF₃ | H | CH₂CH═CF₂ |
| CF₃ | H | Ph-4-CF₃ | H | CH₂CH═CCl₂ |
| CF₃ | H | Ph-4-CF₃ | H | CH₂CF═CF₂ |
| CF₃ | H | Ph-4-CF₃ | H | CH₂CCl═CCl₂ |
| CF₃ | H | Ph-4-CF₃ | H | CH₂C≡CH |
| CF₃ | H | Ph-4-CF₃ | H | CH₂Ph |
| CF₃ | H | Ph-4-CF₃ | H | Si(CH₃)₃ |
| CF₃ | H | Ph-4-CF₃ | H | Si(CH₃)₂Bu-t |
| CF₃ | H | Ph-4-CF₃ | H | Si(CH₃)₂Ph |
| CF₃ | H | Ph-4-OCH₃ | H | H |
| CF₃ | H | Ph-4-OCHF₂ | H | H |
| CF₃ | H | Ph-3-OCF₃ | H | H |
| CF₃ | H | Ph-4-OCF₃ | H | H |
| CF₃ | H | Ph-4-OCF₃ | H | CH₃ |
| CF₃ | H | Ph-4-OCF₃ | H | Et |
| CF₃ | H | Ph-4-OCF₃ | H | n-Pr |
| CF₃ | H | Ph-4-OCF₃ | H | i-Pr |
| CF₃ | H | Ph-4-OCF₃ | H | n-Bu |
| CF₃ | H | Ph-4-OCF₃ | H | CH₂CF₃ |
| CF₃ | H | Ph-4-OCF₃ | H | CH₂OCH₃ |
| CF₃ | H | Ph-4-OCF₃ | H | CH₂OEt |
| CF₃ | H | Ph-4-OCF₃ | H | CH₂SCH₃ |
| CF₃ | H | Ph-4-OCF₃ | H | CH₂CH═CH₂ |
| CF₃ | H | Ph-4-OCF₃ | H | CH₂CH═CF₂ |
| CF₃ | H | Ph-4-OCF₃ | H | CH₂CH═CCl₂ |
| CF₃ | H | Ph-4-OCF₃ | H | CH₂CF═CF₂ |
| CF₃ | H | Ph-4-OCF₃ | H | CH₂CCl═CCl₂ |
| CF₃ | H | Ph-4-OCF₃ | H | CH₂C≡CH |
| CF₃ | H | Ph-4-OCF₃ | H | CH₂Ph |
| CF₃ | H | Ph-4-OCF₃ | H | Si(CH₃)₃ |
| CF₃ | H | Ph-4-OCF₃ | H | Si(CH₃)₂Bu-t |
| CF₃ | H | Ph-4-OCF₃ | H | Si(CH₃)₂Ph |
| CF₃ | H | Ph-4-OCF₂Br | H | H |
| CF₃ | H | Ph-4-OCH₂CF₃ | H | H |
| CF₃ | H | Ph-4-OCF₂CHF₂ | H | H |
| CF₃ | H | Ph-4-OCF₂CHFCl | H | H |
| CF₃ | H | Ph-4-OCF₂CHFBr | H | H |
| CF₃ | H | Ph-4-OCF₂CF₂Br | H | H |
| CF₃ | H | Ph-4-OCF₂CFCl₂ | H | H |
| CF₃ | H | Ph-4-OCF₂CCl₃ | H | H |
| CF₃ | H | Ph-4-OCH₂CF₂CHF₂ | H | H |
| CF₃ | H | Ph-3-OCF₂CHFCF₃ | H | H |
| CF₃ | H | Ph-4-OCF₂CHFCF₃ | H | H |
| CF₃ | H | Ph-4-OCH(CF₃)₂ | H | H |
| CF₃ | H | Ph-4-OCF₂CFBrCF₃ | H | H |
| CF₃ | H | Ph-3-OCF₂CHFOCF₃ | H | H |
| CF₃ | H | Ph-4-OCF₂CHFOCF₃ | H | H |
| CF₃ | H | Ph-4-OCF₂CHFOCF₂CF₂CF₃ | H | H |
| CF₃ | H | Ph-4-OSO₂CH₃ | H | H |
| CF₃ | H | Ph-4-OSO₂CF₃ | H | H |
| CF₃ | H | Ph-3-O(Ph-4-Cl) | H | H |
| CF₃ | H | Ph-4-O(Ph-4-Cl) | H | H |
| CF₃ | H | Ph-4-O(Ph-4-Br) | H | H |
| CF₃ | H | Ph-4-O(Ph-4-CF₃) | H | H |
| CF₃ | H | Ph-4-O(L-45c)Br | H | H |
| CF₃ | H | Ph-4-O(L-45c)CF₃ | H | H |
| CF₃ | H | Ph-3-O(L-45e) | H | H |
| CF₃ | H | Ph-4-O(L-45e) | H | H |
| CF₃ | H | Ph-4-O(L-48b)Br | H | H |
| CF₃ | H | Ph-4-SCH₃ | H | H |
| CF₃ | H | Ph-4-SO₂CH₃ | H | H |
| CF₃ | H | Ph-3-S(Ph-4-Cl) | H | H |
| CF₃ | H | Ph-4-S(Ph-4-Cl) | H | H |
| CF₃ | H | Ph-4-S(Ph-4-Br) | H | H |
| CF₃ | H | Ph-4-S(Ph-4-CF₃) | H | H |
| CF₃ | H | Ph-4-S(L-45c)Br | H | H |
| CF₃ | H | Ph-4-S(L-45c)CF₃ | H | H |
| CF₃ | H | Ph-4-S(L-45e) | H | H |
| CF₃ | H | Ph-4-S(L-48b)Br | H | H |
| CF₃ | H | Ph-4-NO₂ | H | H |
| CF₃ | H | Ph-4-CN | H | H |
| CF₃ | H | Ph-2,3-F₂ | H | H |
| CF₃ | H | Ph-2,4-F₂ | H | H |
| CF₃ | H | Ph-3,4-F₂ | H | H |
| CF₃ | H | Ph-2,5-F₂ | H | H |
| CF₃ | H | Ph-3,5-F₂ | H | H |
| CF₃ | H | Ph-2-Cl-4-F | H | H |
| CF₃ | H | Ph-2-F-3-Cl | H | H |
| CF₃ | H | Ph-3-Cl-4-F | H | H |
| CF₃ | H | Ph-2-F-4-Cl | H | H |
| CF₃ | H | Ph-3-F-4-Cl | H | H |
| CF₃ | H | Ph-2,3-Cl₂ | H | H |
| CF₃ | H | Ph-2,4-Cl₂ | H | H |
| CF₃ | H | Ph-2,5-Cl₂ | H | H |
| CF₃ | H | Ph-3,4-Cl₂ | H | H |
| CF₃ | H | Ph-3,5-Cl₂ | H | H |
| CF₃ | H | Ph-3-Br-4-F | H | H |
| CF₃ | H | Ph-2-F-4-Br | H | H |
| CF₃ | H | Ph-2-F-5-Br | H | H |
| CF₃ | H | Ph-3,4-Br₂ | H | H |
| CF₃ | H | Ph-3,5-Br₂ | H | H |
| CF₃ | H | Ph-3-CH₃-4-F | H | H |
| CF₃ | H | Ph-3-F-4-CH₃ | H | H |
| CF₃ | H | Ph-2-F-5-CH₃ | H | H |
| CF₃ | H | Ph-2,4-(CH₃)₂ | H | H |
| CF₃ | H | Ph-3,4-(CH₃)₂ | H | H |
| CF₃ | H | Ph-2-F-3-CF₃ | H | H |
| CF₃ | H | Ph-3-CF₃-4-F | H | H |
| CF₃ | H | Ph-3-CF₃-4-Cl | H | H |
| CF₃ | H | Ph-2-F-4-CF₃ | H | H |
| CF₃ | H | Ph-3-F-4-CF₃ | H | H |
| CF₃ | H | Ph-2-F-5-CF₃ | H | H |
| CF₃ | H | Ph-3-F-5-CF₃ | H | H |
| CF₃ | H | Ph-2-Cl-4-CF₃ | H | H |
| CF₃ | H | Ph-3,5-(CF₃)₂ | H | H |
| CF₃ | H | Ph-3-Br-4-OCH₃ | H | H |
| CF₃ | H | Ph-3-F-4-OCHF₂ | H | H |
| CF₃ | H | Ph-3-Cl-4-OCHF₂ | H | H |
| CF₃ | H | Ph-3-Br-4-OCHF₂ | H | H |
| CF₃ | H | Ph-3-F-4-OCF₃ | H | H |
| CF₃ | H | Ph-3-Cl-4-OCF₃ | H | H |
| CF₃ | H | Ph-3-Br-4-OCF₃ | H | H |
| CF₃ | H | Ph-3-F-4-OCF₂Br | H | H |
| CF₃ | H | Ph-3-Cl-4-OCF₂Br | H | H |
| CF₃ | H | Ph-3-Br-4-OCF₂Br | H | H |
| CF₃ | H | Ph-3-F-4-OCF₂CHF₂ | H | H |
| CF₃ | H | Ph-3-Cl-4-OCF₂CHF₂ | H | H |
| CF₃ | H | Ph-3-Br-4-OCF₂CHF₂ | H | H |
| CF₃ | H | Ph-3-F-4-OCF₂CHFCl | H | H |
| CF₃ | H | Ph-3-Cl-4-OCF₂CHFCl | H | H |
| CF₃ | H | Ph-3-Br-4-OCF₂CHFCl | H | H |
| CF₃ | H | Ph-3-F-4-OCF₂CHFCF₃ | H | H |
| CF₃ | H | Ph-3-Cl-4-OCF₂CHFCF₃ | H | H |
| CF₃ | H | Ph-3-Br-4-OCF₂CHFCF₃ | H | H |
| CF₃ | H | Ph-3-F-4-OCF₂CHFOCF₃ | H | H |
| CF₃ | H | Ph-3-Cl-4-OCF₂CHFOCF₃ | H | H |
| CF₃ | H | Ph-3-Br-4-OCF₂CHFOCF₃ | H | H |
| CF₃ | H | Ph-3-CH₃-4-OCF₂CHFOCF₃ | H | H |
| CF₃ | H | Ph-3-F-4-OCF₂CHFOCF₂CF₂CF₃ | H | H |
| CF₃ | H | Ph-3-Cl-4-OCF₂CHFOCF₂CF₂CF₃ | H | H |
| CF₃ | H | Ph-3-Br-4-OCF₂CHFOCF₂CF₂CF₃ | H | H |
| CF₃ | H | Ph(-3-OCF₂O-4-) | H | H |
| CF₃ | H | Ph(-3-OCF₂CF₂O-4-) | H | H |
| CF₃ | H | Ph-3-OPh-4-F | H | H |
| CF₃ | H | Ph-3-NO₂-4-F | H | H |
| CF₃ | H | Ph-3-NO₂-4-Cl | H | H |
| CF₃ | H | Ph-2-F-5-NO₂ | H | H |
| CF₃ | H | Ph-3-CN-4-F | H | H |
| CF₃ | H | Ph-2,3,4-F₃ | H | H |
| CF₃ | H | Ph-2,3,5-F₃ | H | H |
| CF₃ | H | Ph-2,4,5-F₃ | H | H |
| CF₃ | H | Ph-3,4,5-F₃ | H | H |
| CF₃ | H | Ph-2,3-F₂-4-CH₃ | H | H |
| CF₃ | H | Ph-2,3-F₂-4-CF₃ | H | H |
| CF₃ | H | Ph-3,4-F₂-5-CF₃ | H | H |
| CF₃ | H | Ph-2-F-3-Cl-5-CF₃ | H | H |
| CF₃ | H | Ph-3,5-Cl₂-4-OCH₃ | H | H |
| CF₃ | H | 1-Naph | H | H |
| CF₃ | H | 2-Naph | H | H |
| CF₃ | H | (L-lb)Br | H | H |
| CF₃ | H | (L-lc)Cl | H | H |
| CF₃ | H | (L-1c)Br | H | H |
| CF₃ | H | (L-1c)I | H | H |
| CF₃ | H | (L-1c)CF₃ | H | H |
| CF₃ | H | (L-2b)Br | H | H |
| CF₃ | H | (L-3b)Cl | H | H |
| CF₃ | H | (L-3b)Br | H | H |
| CF₃ | H | (L-3c)F | H | H |
| CF₃ | H | (L-3c)Cl | H | H |
| CF₃ | H | (L-3c)Br | H | H |
| CF₃ | H | (L-3c)I | H | H |
| CF₃ | H | (L-3c)CF₃ | H | H |
| CF₃ | H | (L-3c)CN | H | H |
| CF₃ | H | L-3d | H | H |
| CF₃ | H | (L-4b)Cl | H | H |
| CF₃ | H | (L-4b)Br | H | H |
| CF₃ | H | (L-4b)CN | H | H |
| CF₃ | H | (L-10b)Cl | H | H |
| CF₃ | H | (L-10b)Br | H | H |
| CF₃ | H | (L-15b)CF₃ | H | H |
| CF₃ | H | (L-16a)CHF₂ | H | H |
| CF₃ | H | (L-16a)CF₂Br | H | H |
| CF₃ | H | (L-17a)Cl | H | H |
| CF₃ | H | (L-21b)Cl | H | H |
| CF₃ | H | (L-21b)Br | H | H |
| CF₃ | H | (L-21b)I | H | H |
| CF₃ | H | (L-21b)CF₃ | H | H |
| CF₃ | H | (L-22b)Cl | H | H |
| CF₃ | H | (L-22b)Br | H | H |
| CF₃ | H | (L-23b)Cl | H | H |
| CF₃ | H | (L-23b)Br | H | H |
| CF₃ | H | (L-23c)Cl | H | H |
| CF₃ | H | (L-23c)Br | H | H |
| CF₃ | H | (L-31a)Cl | H | H |
| CF₃ | H | (L-31a)Br | H | H |
| CF₃ | H | (L-45c)F | H | H |
| CF₃ | H | (L-45c)Cl | H | H |
| CF₃ | H | (L-45c)Br | H | H |
| CF₃ | H | (L-45c)I | H | H |
| CF₃ | H | (L-45c)CF₃ | H | H |
| CF₃ | H | (L-45c)CF₃ | H | CH₃ |
| CF₃ | H | (L-45c)CF₃ | H | Et |
| CF₃ | H | (L-45c)CF₃ | H | n-Pr |
| CF₃ | H | (L-45c)CF₃ | H | i-Pr |
| CF₃ | H | (L-45c)CF₃ | H | n-Bu |
| CF₃ | H | (L-45c)CF₃ | H | CH₂CF₃ |
| CF₃ | H | (L-45c)CF₃ | H | CH₂OCH₃ |
| CF₃ | H | (L-45c)CF₃ | H | CH₂OEt |
| CF₃ | H | (L-45c)CF₃ | H | CH₂SCH₃ |
| CF₃ | H | (L-45c)CF₃ | H | CH₂CH═CH₂ |
| CF₃ | H | (L-45c)CF₃ | H | CH₂CH═CF₂ |
| CF₃ | H | (L-45c)CF₃ | H | CH₂CH═CCl₂ |
| CF₃ | H | (L-45c)CF₃ | H | CH₂CF═CF₂ |
| CF₃ | H | (L-45c)CF₃ | H | CH₂CCl═CCl₂ |
| CF₃ | H | (L-45c)CF₃ | H | CH₂C≡CH |
| CF₃ | H | (L-45c)CF₃ | H | CH₂Ph |
| CF₃ | H | (L-45c)CF₃ | H | Si(CH₃)₃ |
| CF₃ | H | (L-45c)CF₃ | H | Si(CH₃)₂Bu-t |
| CF₃ | H | (L-45c)CF₃ | H | Si(CH₃)₂Ph |
| CF₃ | H | L-45e | H | H |
| CF₃ | H | L-45f | H | H |
| CF₃ | H | (L-46c)F | H | H |
| CF₃ | H | (L-46c)Cl | H | H |
| CF₃ | H | (L-46c)Br | H | H |
| CF₃ | H | (L-46c)I | H | H |
| CF₃ | H | (L-46c)CF₃ | H | H |
| CF₃ | H | (L-46c)OCH₂CF₃ | H | H |
| CF₃ | H | (L-46c)OCH(CF₃)₂ | H | H |
| CF₃ | H | L-46d | H | H |
| CF₃ | H | L-47a | H | H |
| CF₃ | H | L-47d | H | H |
| CF₃ | H | (L-48b)Br | H | H |
| CF₃ | H | (L-50b)Cl | H | H |
| CF₃ | H | (L-50b)Br | H | H |
| CF₃ | H | (L-51b)Cl | H | H |
| CF₃ | —OCF₂— | | H | H |
| CF₃ | —OCF₂— | | H | CH₃ |
| CF₃ | —OCF₂— | | H | CH₂OCH₃ |
| CF₃ | —OCF₂— | | H | Si(CH₃)₃ |
| CF₃ | H | —CF₂O— | | H |
| CF₃ | H | —CF₂O— | | CH₃ |
| CF₃ | H | —CF₂O— | | CH₂OCH₃ |
| CF₃ | H | —CF₂O— | | Si(CH₃)₃ |
| CF₂Cl | H | CF₂O(Ph-4-Cl) | H | H |
| CF₂Cl | H | CH═CH(Ph-4-Cl) | H | H |
| CF₂Cl | H | CH═CH(Ph-4-OCF₃) | H | H |
| CF₂Cl | H | CH═CH(Ph-3,4-C 1₂) | H | H |
| CF₂Cl | H | Ph-A-F | H | H |
| CF₂Cl | H | Ph-4-Cl | H | H |
| CF₂Cl | H | Ph-4-Br | H | H |
| CF₂Cl | H | Ph-4-I | H | H |
| CF₂Cl | H | Ph-4-CF₃ | H | H |
| CF₂Cl | H | Ph-4-OCHF₂ | H | H |
| CF₂Cl | H | Ph-4-OCF₃ | H | H |
| CF₂Cl | H | Ph-4-OCF₂Br | H | H |
| CF₂Cl | H | Ph-4-OCF₂CHF₂ | H | H |
| CF₂Cl | H | Ph-4-OCF₂CHFCl | H | H |
| CF₂Cl | H | Ph-4-OCF₂CHFCF₃ | H | H |
| CF₂Cl | H | Ph-4-OCF₂CHFOCF₃ | H | H |
| CF₂Cl | H | Ph-4-OCF₂CHFOCF₂CF₂CF₃ | H | H |
| CF₂Cl | H | Ph-4-O(L-45g) | H | H |
| CF₂Cl | H | Ph-3,4-Cl₂ | H | H |
| CF₂Cl | H | Ph(-3-OCF₂O-4-) | H | H |
| CF₂Cl | H | Ph(-3-OCF₂CF₂O-4 | H | H |
| CF₂Cl | H | (L-45c)Cl | H | H |
| CF₂Cl | H | (L-45c)Br | H | H |
| CF₂Cl | H | (L-45c)CF₃ | H | H |
| CF₂Cl | H | L-45e | H | H |
| CF₂Cl | H | L-45f | H | H |
| CF₂Cl | H | (L-46c)Cl | H | H |
| CF₂Cl | H | (L-46c)Br | H | H |
| CF₂Cl | H | (L-46c)CF₃ | H | H |
| CF₂Cl | H | L-46d | H | H |
| CFCl₂ | H | Ph-4-OCF₃ | H | H |
| CF₂Br | H | Ph-4-Cl | H | H |
| CF₂Br | H | Ph-4-Br | H | H |
| CF₂Br | H | Ph-4-CF₃ | H | H |
| CF₂Br | H | Ph-4-OCF₃ | H | H |
| CF₂Br | H | Ph-4-OCF₂Br | H | H |
| CF₂Br | H | Ph-4-OCF₂CHFCF₃ | H | H |
| CF₂Br | H | Ph-4-OCF₂CHFOCF₃ | H | H |
| CF₂Br | H | Ph-4-OCF₂CHFOCF₂CF₂CF₃ | H | H |
| CF₂Br | H | Ph-4-O(L-45g) | H | H |
| CF₂Br | H | Ph-3,4-C 1₂ | H | H |
| CF₂Br | H | Ph(-3-OCF₂O-4-) | H | H |
| CF₂Br | H | Ph(-3-OCF₂CF₂O-4-) | H | H |
| CF₂Br | H | (L-45c)CF₃ | H | H |
| CF₂Br | H | L-45f | H | H |
| CF₂Br | H | (L-46c)CF₃ | H | H |
| CF₂Br | H | L-46d | H | H |
| CFClBr | H | (L-45c)CF₃ | H | H |
| CFBr₂ | H | Ph-4-Br | H | H |
| CF₂CHF₂ | H | Ph-4-CF₃ | H | H |
| CF₂CF₃ | H | CF₂O(Ph-4-Cl) | H | H |
| CF₂CF₃ | H | CH═CH(Ph-4-Cl) | H | H |
| CF₂CF₃ | H | CH═CH(Ph-4-OCF₃) | H | H |
| CF₂CF₃ | H | CH═CH(Ph-3,4-Cl₂) | H | H |
| CF₂CF₃ | H | Ph-4-F | H | H |
| CF₂CF₃ | H | Ph-A-Cl | H | H |
| CF₂CF₃ | H | Ph-4-Br | H | H |
| CF₂CF₃ | H | Ph-4-I | H | H |
| CF₂CF₃ | H | Ph-4-CF₃ | H | H |
| CF₂CF₃ | H | Ph-4-OCHF₂ | H | H |
| CF₂CF₃ | H | Ph-4-OCF₃ | H | H |
| CF₂CF₃ | H | Ph-4-OCF₂Br | H | H |
| CF₂CF₃ | H | Ph-4-OCF₂CHF₂ | H | H |
| CF₂CF₃ | H | Ph-4-OCF₂CHFCl | H | H |
| CF₂CF₃ | H | Ph-4-OCF₂CHFCF₃ | H | H |
| CF₂CF₃ | H | Ph-4-OCF₂CHFOCF₃ | H | H |
| CF₂CF₃ | H | Ph-4-OCF₂CHFOCF₂CF₂CF₃ | H | H |
| CF₂CF₃ | H | Ph-4-O(L-45g) | H | H |
| CF₂CF₃ | H | Ph-3,4-Cl₂ | H | H |
| CF₂CF₃ | H | Ph(-3-OCF₂O-4-) | H | H |
| CF₂CF₃ | H | Ph(-3-OCF₂CF₂O-4-) | H | H |
| CF₂CF₃ | H | (L-45c)Cl | H | H |
| CF₂CF₃ | H | (L-45c)Br | H | H |
| CF₂CF₃ | H | (L-45c)CF₃ | H | H |
| CF₂CF₃ | H | L-45e | H | H |
| CF₂CF₃ | H | L-45f | H | H |
| CF₂CF₃ | H | (L-46c)Cl | H | H |
| CF₂CF₃ | H | (L-46c)Br | H | H |
| CF₂CF₃ | H | (L-46c)CF₃ | H | H |
| CF₂CF₃ | H | L-46d | H | H |
| CF₂CF₂Cl | H | Ph-4-OCF₃ | H | H |
| CFClCF₃ | H | (L-45c)CF₃ | H | H |
| CFClCF₃Cl | H | Ph-4-Br | H | H |
| CF₂CF₂Br | H | Ph-4-CF₃ | H | H |
| CFBrCF₃ | H | Ph-4-OCF₃ | H | H |
| CF₂CHFCF₃ | H | (L-45c)CF₃ | H | H |
| CF₂CF₂CF₃ | H | Ph-4-Cl | H | H |
| CF₂CF₂CF₃ | H | Ph-4-Br | H | H |
| CF₂CF₂CF₃ | H | Ph-4-CF₃ | H | H |
| CF₂CF₂CF₃ | H | Ph-4-OCF₃ | H | H |
| CF₂CF₂CF₃ | H | Ph-4-OCF₂Br | H | H |
| CF₂CF₂CF₃ | H | Ph-4-OCF₂CHFCF₃ | H | H |
| CF₂CF₂CF₃ | H | Ph-4-OCF₂CHFOCF₃ | H | H |
| CF₂CF₂CF₃ | H | Ph-4-OCF₂CHFOCF₂CF₂CF₃ | H | H |
| CF₂CF₂CF₃ | H | Ph-4-O(L-45g) | H | H |
| CF₂CF₂CF₃ | H | Ph-3,4-Cl₂ | H | H |
| CF₂CF₂CF₃ | H | Ph(-3-OCF₂O-4-) | H | H |
| CF₂CF₂CF₃ | H | Ph(-3-OCF₂CF₂O-4-) | H | H |
| CF₂CF₂CF₃ | H | (L-45c)CF₃ | H | H |
| CF₂CF₂CF₃ | H | L-45f | H | H |
| CF₂CF₂CF₃ | H | (L-46c)CF₃ | H | H |
| CF₂CF₂CF₃ | H | L-46d | H | H |
| CF(CF₃)₂ | H | Ph-4-Br | H | H |
| CF₂CFClCF₂Cl | H | Ph-4-CF₃ | H | H |
| CF₂CFBrCF₂Cl | H | Ph-4-OCF₃ | H | H |
| CF₂CF₂CF₂CHF₂ | H | (L-45c)CF₃ | H | H |
| CF₂CF₂CF₂CF₃ | H | Ph-4-Br | H | H |
| CF(CF₃)CF₂CF₃ | H | Ph-4-CF₃ | H | H |
| CF₂CF₂CF₂CF₂Cl | H | Ph-4-OCF₃ | H | H |
| T-1 | H | (L-45c)CF₃ | H | H |
| T-2 | H | Ph-4-Br | H | H |
| CF₂OCH₃ | H | Ph-4-Cl | H | H |
| CF₂OCH₃ | H | Ph-4-Br | H | H |
| CF₂OCH₃ | H | Ph-4-CF₃ | H | H |
| CF₂OCH₃ | H | Ph-4-OCF₃ | H | H |
| CF₂OCH₃ | H | Ph-4-OCF₂Br | H | H |
| CF₂OCH₃ | H | Ph-4-OCF₂CHFCF₃ | H | H |
| CF₂OCH₃ | H | Ph-4-OCF₂CHFOCF₃ | H | H |
| CF₂OCH₃ | H | Ph-4-OCF₂CHFOCF₂CF₂CF₃ | H | H |
| CF₂OCH₃ | H | Ph-4-O(L-45g) | H | H |
| CF₂OCH₃ | H | Ph-3,4-Cl₂ | H | H |
| CF₂OCH₃ | H | Ph(-3-OCF₂O-4-) | H | H |
| CF₂OCH₃ | H | Ph(-3-OCF₂CF₂O-4-) | H | H |
| CF₂OCH₃ | H | (L-45c)CF₃ | H | H |
| CF₂OCH₃ | H | L-45f | H | H |
| CF₂OCH₃ | H | (L-46c)CF₃ | H | H |
| CF₂OCH₃ | H | L-46d | H | H |
| CF₂SCH₃ | H | Ph-4-Cl | H | H |
| CF₂SCH₃ | H | Ph-4-Br | H | H |
| CF₂SCH₃ | H | Ph-4-CF₃ | H | H |
| CF₂SCH₃ | H | Ph-4-OCF₃ | H | H |
| CF₂SCH₃ | H | Ph-4-OCF₂Br | H | H |
| CF₂SCH₃ | H | Ph-4-OCF₂CHFCF₃ | H | H |
| CF₂SCH₃ | H | Ph-4-OCF₂CHFOCF₃ | H | H |
| CF₂SCH₃ | H | Ph-4-OCF₂CHFOCF₂CF₂CF₃ | H | H |
| CF₂SCH₃ | H | Ph-4-O(L-45g) | H | H |
| CF₂SCH₃ | H | Ph-3,4-Cl₂ | H | H |
| CF₂SCH₃ | H | Ph(-3-OCF₂O-4-) | H | H |
| CF₂SCH₃ | H | Ph(-3-OCF₂CF₂O-4-) | H | H |
| CF₂SCH₃ | H | (L-45c)CF₃ | H | H |
| CF₂SCH₃ | H | L-45f | H | H |
| CF₂SCH₃ | H | (L-46c)CF₃ | H | H |
| CF₂SCH₃ | H | L-46d | H | H |
| CF₂SPr-n | H | Ph-4-CF₃ | H | H |
| CF₂SPr-I | H | Ph-4-OCF₃ | H | H |

The compound of the present invention can prevent from and exterminate either of harmful insects with a low concentration such as the so-called agricultural harmful insects which injure agricultural and horticultural crops and trees, the so-called harmful insects against domestic animals which are parasitic on domestic animals and domestic dowls, the so-called hygiene harmful insects which provide various bad influences on a human life environment such as houses, etc., the so-called harmful insects against stored grains which injure grains stored in a storehouse, and mites, nematodes, mollusks and crustaceans which generate in the same situation and injure.

In the insects, mites, nematodes, molluscs and crustaceans which can be prevented and exterminated by using the compound of the present invention, there may be specifically mentioned, for example,

Lepidoptera harmful insects such as diamondback moth (Plutella xylostella), black cutworm (Agrotis ipsilon), Turnip moth (Agrous segetum), corn earworm (Helicoverpa armigera), Oriental tobacco budworm (Helicoverpa assulta), cotton bollworm (Helicoverpa zea), tobacco budworm (Heliothis virescens), cabbage armyworm (Mamestra brassicae), green rice caterpillar (Naranga aenescens), beet semilooper (Autographa nigrisigna), rice armyworm (Mythimna separata), beet armyworm (Spodoptera exigua), common cutworm (Spodoptera litura), cotton leafworm (Spodoptera littoralis), fall armyworm (Spodoptera frugiperda), southern armyworm (Spodoptera eridania), tomato hornworm (Manduca quinquemaculata), tobacco hornworm (Manduca sexta), grapeberry moth (Endopiza viteana), apple lyonetid (Lyonetia prunifoliella malinella), apple leafminer (Phyllonorycter ringoniella), citrus leafminer (Phyllocnistis citrella), pink bollworm (Pectinophora gossypiella), peach fruit moth (Carposina sasakii), summer fruit tortrix (Adoxophyes orana faciata), smaller tea tortrix (Adoxophyes honmai), oriental tea tortrix (Homona magnamina), codling moth (Cydla pomonella), Oriental fruit moth (Grapholita molesta), rice stem borer (Chilo suppressalis), rice leafroller (Cnaphalocrocis medinalis), cabbage webworm (Hellula undalis), European corn borer (Ostrinia nubilalis), soybean looper (Pseudoplusia includens), cabbage looper (Trichoplusia ni), fall wedworm (Hyphantria cunea), common white (Pieris rapae crucivora), rice skipper (Pamara guttata), etc.,

Coleoptera harmful insects such as cupreous chafer beetle (Anomala cuprea), soybean beetle (Anomala rufocuprea), Japanese beetle (Popillia japonica), Colorado potato beetle (Lepinotarsa decemlineata), Mexican bean beetle (Epilachna varivestis), sugarcane wireworm (Melanotus tamsuyensis), cigarette beetle (Lasioderma serricorne), Himehirata Nitidulidae (Epuraea domina), twentyeight-spotted ladybird (Epilachna vigintioctopunctata), yellow mealworm (Tenebrio molitor), red flour beetle (Tribolium castaneum), whitespotted longicom beetle (Anoplophora malasiaca), japanese pine sawyer (Monochamus alternatus), azuki bean weevil (Callosobruchus chinensis), cucurbit leaf beetle (Aulacophora femoralis), rice leaf beetle (Oulema oryzae), striped flea beetle (Phyllotreta striolata), sweetpotato weevil (Cylas formicarius), boll weevil (Anthonomus grandis), rice curculio (Echinocnemus squameus), alfalfa weevil (Hypera postica), rice water weevil (Lissohoptrus oryzophilus), maize weevil (Sitophilus zeamais), hunting billbug (Sphenophorus venatus vestitus), granary weevil (Sitophilus granarius), southern corm rootworm (Diabrotica undecimpunctata), western corn rootworm (Diabrotica virgifera), northern corn rootworm (Diabrotica barberi), rove beetle (Paedenus fuscipes), etc.,

Hemiptera harmful insects such as cabbage bug (Eurydema rugosum), whitespotted bug (Eysarcoris ventralis), brown marmorated stink bug (Halyomorpha halys), southern green stink bug (Nezara viridula), rice bug (Leptocorisa chinensis), bean bug (Riptortus clavatus), small wing gourd bug (Togo hemiptenus), tarnished plant bug (Lygus lineolaris), cotton fleahopper (Pseudatomoscelis seriatus), azalea lace bug (Stephanitis pyrioides), grape leafhopper (Epiacanthus stramineus), tea green leafhopper (Empoasca onukii), potato leafhopper (Empoasca fabae), green rice leafhopper (Nephotettix cinctinceps), small brown planthopper (Laodelphax striatella), brown rice planthopper (Nilaparvata lugens), whitebacked rice planthopper (Sogatella furcifera), Asiatic citrus psylla (Diaphorina citri), pear psylla (Psylla pyrisuga), silverleaf whitefly (Bemisia argentifolii), sweetpotato whitefly (Bemisia tabaci), citrus whitefly (Dialeurodes citri), greenhouse whitefly (Trialeurodes vaporariorum), cotton aphid (Aphis gossypii), spiraea aphid (Aphis spiraecola), green peach aphid (Myzus persicae), giant margarodid mealybug (Drosicha corpulenta), cottony cushion scale (Icerya purchasi), citrus mealybug (Planococcus citri), cornstock mealybug (Pseudococcus comstocki), red wax scale (Ceroplastes rubens), arrowhead scale (Unaspis yanonensis), bed bug (Cimex lectularius), etc.,

Thysanoptera harmful insects such as western flower thrips (Frankliniella occidentalis), flower thrips (Frankliniella intonsa), yellow tea thrips (Scirtothrips dorsalis), melon thrips (Thrips palmi), onion thrips (Thrips tabaci), etc.,

Diptera harmful insects such as oriental fruit fly (Dacus dorsalis), melon fly (Dacus cucurbitae), mediterranean fruit fly (Ceratitis capitata), rice leaf miner (Hydrellia griseola), bryony leaf miner (Liriomyza bryoniae), serpentine leaf miner (Liriomyza trifolii), seedcom maggot (Delia platura), apple maggot (Rhagoletis pomonella), hessian fly (Mayetiola destructor), house fly (Musca domestica), stable fly (Stomoxys calcitrans), sheep ked (Melophagus ovinus), northern cattle grub (Hypoderma bovis), common cattle grub (Hypoderma lineatum), sheep nasal bot fly (Oestrus ovis), tsetse fly (Glossina palpalis, Glossina morsitans), yellow-leg giant gnat (Prosimulium yezoensis), cattle hoursefly (Tabanus trigonus), filter fly or bathroom fly (Telmatoscopus albipunctatus), tokunaganuka mosquito (Leptoconops nipponensis), brown house mosquito (Culex pipiens pallens), yellow fever mosquito (Aedes aegypti), asian tiger mosquito (Aedes albopictus), chinese spotted mosquito (Anopheles hyracanus sinesis), etc.,

Hymenoptera harmful insects such as chestnut sawfly (Apethymus kuri), turnip sawfly (Athalia rosae japonensis), european pine sawfly (Neodiprion sertifer), american army ants (Eciton burchelli, Eciton schmitti), red carpenter ant (Camponotus japonicus), giant asian homet (Vespa mandarina), bulldog ants (Myrmecia spp.), fire ants (Solenopsis spp.), pharaoh ant (Monomorium pharaonis), etc.,

Blattaria harmful insects such as smokybrown cockroach (Periplaneta fuliginosa), Japanese cockroach (Periplaneta japonica), German cockroach (Blattella germanica), etc.,

Orthoptera harmful insects such as emma field cricket (Teleogryllus emma), mole cricket (Gryllotalpa orientalis), migratory locust (Locusta migratoria), rice grasshopper (Oxya yezoensis), desert locust (Schistocerca gregaria), etc.,

Isoptera harmful insects such as formosan subterranean termite (Coptotermes formosanus), Japanese termite (Reticulitermes speratus), Taiwanese termite (Odontotermes formosanus), etc.,

Isoptera harmful insects such as cat flea (Ctenocephalides felis), human flea (Pulex irritans), Oriental rat flea (Xenopsylla cheopis), etc.,

Mallophage harmful insects such as chicken body louse (Menacanthus stramineus), cattlebiting louse (Bovicola bovis), etc.,

Anoplura harmful insects such as shortnosed cattle louse (Haematopinus eurysternus), pig louse (Haematopinus suis), longnosed cattle louse (Linognathus vituli), tubercle-bearing louse (Solenopotes capillatus), etc.,

Tetranychidae such as citrus red mite (Panonychus citri), European red mite (Panonychus ulmi), kanzawa spider mite (Tetranychus kanzawai), two-spotted spider mite (Tetranychus urticae), etc.,

Eriophyidae such as pink tea rust mite (Acaphylla theae), pink citrus rust mite (Aculops pelekassi), Japanese pear rust mite (Eriophyes chibaensis), wheat curl mite (Aceria tulipae), etc.,

Tarsonemidae such as broad mite (Polyphagotarsonemus latus), cyclamen mite (Steneotarsonemus pallidus), etc.,

Acaridae such as mould mite (Tyrophagus putrescentiae), bulb mite (Rhizoglyphus robini), etc.,

Varroidae such as honeybee varroa mite (Varroa jacobsoni), etc.,

Metastigmata such as southem cattle tick (Boophilus microplus), bush tick (Haemaphysalis longicomis), etc.,

Scab mites such as sheep scab mite (Psoroptes ovis), etc.,

Sarcoptes scabiei such as scabies mite (Sarcoptes scabiei), etc.,

Crustaceans such as pill bug (Armadillidium vulgare), etc.,

Nematodes such as cobb's root-lesion nematode (Pratylenchus penetrans), walnut root-lesion nematode (Pratylenchus vulnus), potato cyst nematode (Globodera rostochiensis), soybean cyst nematode (Heterodera glycines), northern root-knot nematode (Meloidogyne hapla), southern root-knot nematode (Meloidogyne incognita), pinewood nematode (Bursaphelenchus lignicolus), etc., Mollusks such as channeled apple snail (Ponacea canaliculata), slug (Incilaria bilineatum), Korean round snail (Acusta despecta sieboldiana), Japanese land snail (Euhadra peliomphala), etc.,

but the present invention is not limited by these alone.

Moreover, the compound of the present invention is also effective to harmful insects which are improved in resistance against already presenting insecticides such as organophosphurus type compounds, carbamate type compounds or pyrethroid type compounds, etc.

That is, the compound of the present invention can be effectively present and exterminate harmful insects of Orthoptera, Order Thysanoptera, Hemiptera, Lepidoptera, Coleoptera, Hymenoptera, Thysanoptera, Blattaria, Isoptera, Isoptera, mites and lice, and nematodes with a low concentration. On the other hand, the compound of the present invention has extremely useful characteristics that it causes substantially no bad effect against mammals, foshes, crustaceans and useful insects.

For the purpose of using the compound of the present invention, by mixing with a suitable solid carrier or a liquid carrier, and further, if desired, by adding a surfactant, a penetrant, a spreading agent, a thickening agent, an antifreezing agent, a binder, a non-caking agent, a discipient, an antifoaming agent, an antiseptic agent and a decomposition preventing agent, etc., it can be practically applied in an optional formulations such as soluble concentrate, emulsifiable concentrate, wettable powder, water soluble powder, water dispersible granule, water soluble granule, suspension concentrate, concentrated emulsion, suspoemulsion, microemulsion, dustable powder, granule, tablet and emulsifiable gel, etc. Also, in the viewpoints of labor saving and improvement in safety, the above-mentioned formulations in an optional form are encapsulated in a water-soluble container such as a bag of a water-soluble capsule and water-soluble film, etc. and applied practically.

As the solid carrier, there may be mentioned, for example, natural minerals such as quartz, calcite, sepiolite, dolomite, chalk, kaolinite, pyrophyylite, sericite, halocite, metahalocite, Kibushi clay, Gaerome clay, kaolin, zeeklite, allophane, white sand (loam), mica, talc, bentonite, active china clay, acidic china clay, pumice, attapulgite, zeolite and diatomaceous earth, etc., calcined products of natural minerals such as calcined clay, perlite, white sand balloon (loam balloon), vermiculite, attapulgus clay and calcined diatomaceous earth, etc., inorganic salts such as magnesium carbonate, calcium carbonate, sodium carbonate, sodium hydrogen carbonate, ammonium sulfate, sodium sulfate, magnesium sulfate, diammonium hydrogen phosphate, ammonium dihydrogen phosphate and potassium chloride, etc., saccharides such as glucose, fructose, sucrose and lactose, etc., polysaccharides such as starch, powder cellulose and dextrin, etc., organic materials such as urea, urea derivatives, benzoic acid and a salt of benzoic acid, etc., plants such as wood powder, cork powder, corn head stem, walnut shell and tobacco stem, etc., fly ash, white carbon (e.g., hydrated synthetic silica, anhydrous synthetic silica and hydrated synthetic silicate, etc.) and feritilizers, etc.

As the liquid carrier, there may be mentioned, for example, aromatic hydrocarbons such as xylene, alkyl(C₉ or C₁₀, etc.)benzene, phenylxylylethane and alkyl(C₁ or C₃, etc.)naphthalene, etc., aliphatic hydrocarbons such as machine oil, normal paraffin, isoparaffin and naphthene, etc., a mixture of aromatic hydrocarbons and aliphatic hydrocarbons such as kerosene, etc., alcohols such as ethanol, isopropanol, cyclohexanol, phenoxyethanol and benzylalcohol, etc., polyvalent alcohols such as ethylene glycol, propyleneglycol, diethylene glycol, hexylene glycol, polyethylene glycol and polypropyleneglycol, etc., ethers such as propyl cellosolve, butyl cellosolve, phenyl cellosolve, propyleneglycol monomethyl ether, propyleneglycol monoethyl ether, propyleneglycol monopropyl ether, propyleneglycol monobutyl ether and propyleneglycol monophenyl ether, etc., ketones such as acetophenone, cyclohexanone and γ-butyrolactone, etc., esters such as aliphatic acid methyl ester, dialkyl succinate, dialkyl glutamate, dialkyl adipate and dialkyl phthalate, etc., acid amides such as N-alkyl(C₁, C₈ or C₁₂, etc.)pyrrolidone, etc., oil and fats such as soybean oil, linseed oil, rapeseed oil, coconut oil, cottonseed oil and caster oil, etc., dimethylsulfoxide and water.

These solid and liquid carriers may be used alone or in combination of two or more kinds in combination.

As the surfactant, there may be mentioned, for example, nonionic surfactants such as polyoxyethylene alkyl ether, polyoxyethylene alkyl (mono- or di-)phenyl ether, polyoxyethylene (mono-, di- or tri-)styrylphenyl ether, polyoxyethylene polyoxypropylene block copolymer, polyoxyethylene fatty acid (mono- or di-)ester, sorbitan fatty acid ester, polyoxyethylene sorbitan fatty acid ester, caster oil-ethylene oxide adducts, acetylene glycol, acetylene alcohol, ethylene oxide adducts of acetylene glycol, ethylene oxide adducts of acetylene alcohol and alkyl glycoside, etc., anionic surfactants such as alkyl sulfate, alkylbenzenesulfonate, lignine sulfonate, alkylsulfosuccinate, naphthalene sulfonate, alkylnaphthalene sulfonate, formalin condensate salt of naphthalene sulfonic acid, formalin condensate salt of alkylnaphthalene sulfonic acid, polyoxyethylene alkyl ether sulfate or phosphate, polyoxyethylene (mono- or di-)alkylphenyl ether sulfate or phosphate, polyoxyethylene (mono-, di- or tri-)styrylphenyl ether sulfate or phosphate, polycarboxylate (e.g., polyacryaltes, polymaleates and copolymer materials of maleic acid and olefin, etc.) and polystyrenesulfonate, etc., cationic surfactants such as alkylamine salt and alkyl quaternary ammonium salt, etc., amphoteric surfactants such as amino acid type and betaine type, etc., silicone type surfactants and fluorine type surfactants.

A content of these surfactants is not specifically limited, and it is desirably in the range of 0.05 to 20 parts by weight in general based on 100 parts by weight of the preparation according to the present invention. Also, these surfactants may be used alone or in combination of two or more kinds in combination.

A dose of the compound of the present invention to be applied may vary depending on the place to be applied, time to be applied, method to be applied, crops to cultivate, etc., and in general, it is suitable in an amount of about 0.005 to 50 kg or so per a hectare (ha) as an amount of the effective ingredient.

Next, Formulation examples of the preparation when the compound of the present invention is used are shown below. Provided that Formulation examples of the present invention are not limited by these. Incidentally, in the following Formulation examples, all “part(s)” mean part(s) by weight.

[Wettable Powder]

| | | |
|---|---|---|
| Compound of the present invention | 0.1 to 80 | parts |
| Solid carrier | 5 to 98.9 | parts |
| Surfactant | 1 to 10 | parts |
| Others | 0 to 5 | parts |

As other components, there may be mentioned, for example, a non-caking agent, a decomposition preventing agent, and the like.

[Emulsifiable Concentrate]

| | |
|---|---|
| Compound of the present invention | 0.1 to 30 parts |
| Liquid carrier | 45 to 95 parts |
| Surfactant | 4.9 to 15 parts |
| Others | 0 to 10 parts |

As other components, there may be mentioned, for example, a spreading agent, a decomposition preventing agent, and the like.

[Suspension Concentrate]

| | | |
|---|---|---|
| Compound of the present invention | 0.1 to 70 | parts |
| Liquid carrier | 15 to 98.89 | parts |
| Surfactant | 1 to 12 | parts |
| Others | 0.01 to 30 | parts |

As other components, there may be mentioned, for example, an antifreezing agent, a thickening agent, and the like.

[Water Dispersible Granule]

| | | |
|---|---|---|
| Compound of the present invention | 0.1 to 90 | parts |
| Solid carrier | 0 to 98.9 | parts |
| Surfactant | 1 to 20 | parts |
| Others | 0 to 10 | parts |

As other components, there may be mentioned, for example, a binder, a decomposition preventing agent, and the like.

[Soluble Concentrate]

| | | |
|---|---|---|
| Compound of the present invention | 0.01 to 70 | parts |
| Liquid carrier | 20 to 99.99 | parts |
| Others | 0 to 10 | parts |

As other components, there may be mentioned, for example, an antifreezing agent, a spreading agent, and the like.

[Granule]

| | | |
|---|---|---|
| Compound of the present invention | 0.01 to 80 | parts |
| Solid carrier | 10 to 99.99 | parts |
| Others | 0 to 10 | parts |

As other components, there may be mentioned, for example, a binder, a decomposition preventing agent, and the like.

[Dustable Powder]

| | | |
|---|---|---|
| Compound of the present invention | 0.01 to 30 | parts |
| Solid carrier | 65 to 99.99 | parts |
| Others | 0 to 5 | parts |

As other components, there may be mentioned, for example, a drift preventing agent, a decomposition preventing agent, and the like.

Next, Formulation examples using the compound of the present invention as an effective ingredient are described in more detail, but the present invention is not limited by these.

Incidentally, in the following Formulation examples, “part(s)” means part(s) by weight.

### FORMULATION EXAMPLE 1

### Wettable Powder

| | |
|---|---|
| Present compound No. 1-038 | 20 parts |
| Pyrophyylite | 74 parts |
| Solpol 5039 | 4 parts |
| (a mixture of a nonionic surfactant and an anionic surfactant: | |
| available from TOHO Chemical Industry Co., LTD, Tradename) | |
| CARPREX #80D | 2 parts |
| (Synthetic hydrated silicic acid: available from Shionogi & Co., | |
| Ltd., Tradename) | |

The above materials are uniformly mixed and pulverized to make wettable powder.

### FORMULATION EXAMPLE 2

### Emulsion

| | |
|---|---|
| Present compound No. 1-038 | 5 parts |
| xylene | 75 parts |
| N-methylpyrrolidone | 15 parts |
| Solpol 2680 | 5 parts |
| (a mixture of a nonionic surfactant and an anionic surfactant: | |
| available from TOHO Chemical Industry Co., LTD, Tradename) | |

The above materials are uniformly mixed to make emulsifiable concentrate.

### FORMULATION EXAMPLE 3

### Suspension Concentrate

| | | |
|---|---|---|
| Present compound No. 1-038 | 25 | parts |
| Agrisol S-710 | 10 | parts |
| (a nonionic surfactant: available from KAO | | |
| CORPORATION, Tradename) | | |
| Lunox 1000C | 0.5 | part |
| (an anionic surfactant: available from TOHO Chemical | | |
| Industry Co., LTD. Tradename) | | |
| Xanthan gum | 0.2 | part |
| Water | 64.3 | parts |

The above materials are uniformly mixed and pulverized, and then, wet pulverized to make suspension concentrate.

### FORMULATION EXAMPLE 4

### Water Dispersible Granule

| | |
|---|---|
| Present compound No. 1-038 | 75 parts |
| HITENOL NE-15 | 5 parts |
| (an anionic surfactant: available from DAI-ICHI KOGYO | |
| SEIYAKU CO., LTD., Tradename) | |
| VANILLEX N | 10 parts |
| (an anionic surfactant: available from Nippon Paper | |
| Chemicals Co., Ltd., Tradename) | |
| CARPREX #80D | 10 parts |
| (Synthetic hydrated silicic acid: available from | |
| Shionogi & Co., Ltd., Tradename) | |

The above materials are uniformly mixed and pulverized, and then, a small amount of water is added to the mixture and the resulting mixture is mixed under stirring, granulated by an extrusion granulator, and dried to make water dispersible granule.

### FORMULATION EXAMPLE 5

### Granule

| | |
|---|---|
| Present compound No. 1-038 | 5 parts |
| Bentonite | 50 parts |
| Talc | 45 parts |

The above materials are uniformly mixed and pulverized, and then, a small amount of water is added to the mixture and the resulting mixture is mixed under stirring, granulated by an extrusion granulator, and dried to make granule.

### FORMULATION EXAMPLE 6

### Dustable Powder

| | |
|---|---|
| Present compound No. 1-038 | 3 parts |
| CARPREX #80D | 0.5 parts |
| (Synthetic hydrated silicic acid: available from | |
| Shionogi & Co., Ltd., Tradename) | |
| Caolinite | 95 parts |
| Diisopropyl phosphate | 1.5 parts |

The above materials are uniformly mixed and pulverized to make dustable powder.

For the purpose of use, the above-mentioned formulations are spread by diluting to 1 to 10000-folds with water, or directly spread without dilution.

Also, when the compound of the present invention is used as an agricultural chemicals, it may be mixed with other kinds of herbicides, various kinds of insecticides, acaricides, nematocides, fungicides, vegetable growth regulators, synergists, fertilizers, soil improvers, etc., and applied, at the time of preparing the formulation or at the time of spreading, if necessary.

In particular, by mixing with the other agricultural chemicals or plant hormones and applying the mixture, it can be expected that a cost is reduced due to reduction in a dose to be applied, enlargement in insecticidal spectrum or higher prevention and extinction effect of noxious organisms due to synergistic effect by mixing agricultural chemicals. At this time, it is possible to use the compound with a plural number of the conventionally known agricultural chemicals in combination simultaneously. As the kinds of the agricultural chemicals to be used in admixture with the compound of the present invention, there may be mentioned, for example, the compounds described in Farm Chemicals Handbook, 1999^{th} Edition and the like. Specific examples of the general names can be enumerated below, but the invention is not necessarily limited by these alone.

Fungicide: acibenzolar-S-methyl, acylaminobenzamide, ambam (amobam), ampropylfos (ampropylos), anilazine, azaconazole, azoxystrobin, benalaxyl, benodanil, benomyl, benthiazole, benzamacril, binapacryl, biphenyl, bitertanol, bethoxazine, bordeaux mixture, blasticidin-S, bromoconazole, bupirimate, buthiobate, calcium polysulfide, captafol, captan, copper oxychloride, carpropamid, carbendazim, carboxin, CGA-279202 (Test name), chinomethionat, chlobenthiazone, chlorfenazol, chloroneb, chlorothalonil, chlozolinate, cufraneb, cymoxanil, cyproconazol, cyprodinil, cyprofuram, dazomet, debacarb, dichlorophen, diclobutrazol, dichlorfluanid, diclomedine, dicloran, diethofencarb, diclocymet, difenoconazole, diflumetorim, dimethirimol, dimethomorph, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridianole (etridiazole), famoxazone (famoxadone), fenarimol, febuconazole, fenamidone, fendazosulam, fenfuram, fenhexamid, fenpiclonil, fenpropidin, fenpropimorph, fentin, ferbam, ferimzone, fluazinam, fludioxonil, fluoroimide, fluquinconazole, flusilazole, flusulfamide, flutolanil, flutriafol, folpet, fosetyl-aluminium, fuberidazole, furalaxyl, furametpyr, guazatine, hexachlorobenzene, hexaconazole, hymexazol, imazalil, imibenconazole, iminoctadine, ipconazole, iprobenfos, iprodione, isoprothiolane, iprovalicarb, kasugamycin, kresoxim-methyl, mancopper, mancozeb, maneb, mepanipyrim, mepronil, metalaxyl, metconazole, methasulfocarb, metiram, metominostrobin, myclobutanil, MTF-753 (Test name), nabam, nickel bis(dimethyldithiocarbamate), nitrothal-isopropyl, nuarimol, NNF-9425 (Test name), octhilinone, ofurace, oxadixyl, oxycarboxin, oxpoconazole fumarate, pefurzoate, penconazole, pencycuron, phthalide, piperalin, polyoxins, potassium hydrogen carbonate, probenazole, prochloraz, procymidone, propamocarb hydrochloride, propiconazole, propineb, pyrazophos, pyrifenox, pyrimethanil, pyroquilon, quinomethionate, quinoxyfen, quintozene, RH7281 (Test name), sodium hydrogen carbonate, sodium hypochlorite, sulfur, spiroxamine, tebuconazole, tecnazene, tetraconazole, thiabendazole, thiadiazin/milneb, thifluzamide, thiophanate-methyl, thiram, tolclofos-methyl, tolyfluranid, triadimefon, triadimenol (toriadimenol), triazoxide, tricyclazole, tridemorph, triflumizole, triforine, triticonazole, validamycin, vinclozolin, zinc sulfate, zineb, ziram and shiitake mushroom hyphae extract, etc.

Bactericide: streptomycin, tecloftalam, oxytetracyclin and oxolinic acid, etc.

Nematocide: aldoxycarb, cadusafos, fosthiazate, fosthietan, oxamyl and fenamiphos, etc.

Acaricide: acequinocyl, amitraz, bifenazate, bromopropylate, chinomethionat, chlorobezilate, clofentezine, cyhexatine, dicofol, dienochlor, etoxazole, fenazaquin, fenbutatin oxide, fenpropathrin, fenproximate, halfenprox, hexythiazox, milbemectin, propargite, pyridaben, pyrimidifen and tebufenpyrad, etc.

Insecticide: abamectin, acephate, acetamipirid, aldicarb, allethrin, azinphosmethyl, bendiocarb, benfuracarb, bensultap, bifenthrin, buprofezin, butocarboxim, carbaryl, carbofuran, carbosulfan, cartap, chlorfenapyr, chlorpyrifos, chlorfenvinphos, chlorfluazuron, clothianidin, chromafenozide, chlorpyrifos-methyl, cycloprothrin, cyfluthrin, beta-cyfluthrin, cypermethrin, cyromazine, cyhalothrin, lambda-cyhalothrin, deltamethrin, diafenthiuron, diazinon, diacloden, diflubenzuron, dimethylvinphos, diofenolan, disulfoton, dimethoate, emamectin-benzoate, EPN, esfenvalerate, ethiofencarb, ethiprole, etofenprox, etrimfos, fenitrothion, fenobucarb, fenoxycarb, fenpropathrin, fenvalerate, fipronil, fluacrypyrim, flucythrinate, flufenoxuron, flufenprox, tau-fluvalinate, fonophos, formetanate, formothion, furathiocarb, halofenozide, hexaflumuron, hydramethylnon, imidacloprid, isofenphos, indoxacarb, isoprocarb, isoxathion, lufenuron, malathion, metaldehyde, methamidophos, methidathion, methacrifos, metalcarb, methomyl, methoprene, methoxychlor, methoxyfenozide, monocrotophos, muscalure, nidinotefuran, nitenpyram, omethoate, oxydemeton-methyl, oxamyl, parathion, parathion-methyl, permethrin, phenthoate, phoxim, phorate, phosalone, phosmet, phosphamidon, pirimicarb, pirimiphos-methyl, profenofos, protrifenbute, pymetrozine, pyraclofos, pyriproxyfen, rotenone, sulprofos, silafluofen, spinosad, sulfotep, tebfenozide, teflubenzuron, tefluthorin, terbufos, tetrachlorvinphos, thiacloprid, thiocyclam, thiodicarb, thiamethoxam, thiofanox, thiometon, tolfenpyrad, tralomethrin, trichlorfon, triazuron, triflumuron and vamidothion, etc.

### EXAMPLE

In the following, the present invention will be explained in more detail by specifically referring to Synthetic examples and Test Examples of the compound of the present invention as Examples, but the present invention is not limited by these.

### SYNTHETIC EXAMPLES

### Synthetic Example 1

### N′-[4-[1-hydroxy-1-(5-trifluoromethylpyridin-2-yl)ethyl]-2-methylphenyl]-3-iodo-N²-isopropylphthalic diamide (Present Compound No. 2-001)

### Step 1; Preparation of 2-cyano-5-trifluoromethylpyridine

To 150 ml N,N-dimethylformamide solution containing 15.0 g of 2-chloro-5-trifluoromethylpyridine were added 19.4 g of zinc cyanide and 9.6 g of tetrakistriphenylphosphine palladium, and the mixture was stirred under nitrogen atmosphere at 80° C. for 3 hours. After completion of the reaction, the reaction mixture was cooled to room temperature by allowing to stand, poured into 300 ml of dil. aqueous ammonia, and extracted with 300 ml of diethyl ether. The organic layer was washed with 200 ml of a saturated saline solution, then dried over anhydrous magnesium sulfate, the solvent was removed under reduced pressure, and the residue was purified by distillation under reduced pressure (96.0 to 99.0° C./40 mmHg) to obtain 10.9 g of the objective product as white crystals.

Melting point: 36.0 to 38.0° C.

¹H NMR (CDCl₃, Me₄Si, 300 MHz) δ 9.00 (bs, 1H), 8.13 (dd, J=8.3, 2.1 Hz, 1H), 7.87 (d, J=8.3 Hz, 1H).

### Step 2; Preparation of 2-acetyl-5-trifluoromethylpyridine

Under nitrogen atmosphere, to 40 ml of a tetrahydrofuran solution containing 4.7 g of 2-cyano-5-trifluoromethylpyridine was added dropwise 35.0 ml of a tetrahydrofuran solution containing methyl magnesium bromide (0.93 M) at −78° C. under stirring, and after completion of the dropwise addition, the temperature of the mixture was raised to room temperature, and stirring was continued at room temperature for further 1 hour. After completion of the reaction, the reaction mixture was poured into 100 ml of 2N hydrochloric acid, and extracted with 200 ml of ethyl acetate. The organic layer was dehydrated and dried by saturated saline solution and then anhydrous magnesium sulfate in this order, and the solvent was removed under reduced pressure to obtain 4.5 g of the objective product as brownish oily substance.

¹H NMR (CDCl₃, Me₄Si, 300 MHz) δ 8.95 (bs, 1H), 8.16 (d, J=8.4 Hz, 1H), 8.08 (dd, J=8.4, 2.1 Hz, 1H), 2.76 (s, 3H).

### Step 3; Preparation of t-butyl 4-[1-hydroxy-1-(5-trifluoromethylpyridin-2-yl)ethyl]-2-methylcarbanilate

Under nitrogen atmosphere, to 40 ml of a t-butylmethylether solution containing 3.0 g of t-butyl 4-iodo-2-methylcarbanilate was added dropwise 12.5 ml of n-butyl lithium (1.57M hexane solution) at −50° C. under stirring, and after completion of the dropwise addition, the temperature of the mixture was raised to 0° C., and the mixture was stirred for further 30 minutes. Then, the reaction mixture was cooled to −78° C., 1.7 g of 2-acetyl-5-trifluoromethylpyridine was added thereto, the temperature of the mixture was gradually raised to 0° C., and stirring was continued at the same temperature for further 14 hours. After completion of the reaction, to the reaction mixture was added 100 ml of a saturated aqueous ammonium chloride solution, the organic layer was collected by separation, and the aqueous layer was extracted with 100 ml of ethyl acetate. The organic layers were combined, dried over anhydrous magnesium sulfate, and the solvent was removed under reduced pressure. The residue was purified by silica gel chromatography eluting with ethyl acetate-hexane (1:9 to 2:3) to obtain 1.3 g of the objective product as brown solid.

Melting point: 132.0 to 134.5° C.

¹H NMR (CDCl₃, Me₄Si, 300 MHz) δ 8.79 (bs, 1H), 7.86 (dd, J=8.3, 2.1 Hz, 1H), 7.77 (d, J=8.3 Hz, 1H), 7.42 (d, J=8.3 Hz, 1H), 7.2-7.3 (m, 2H), 6.24 (bs, 1H), 5.23 (s, 1H), 2.22 (s, 3H), 1.92 (s, 3H), 1.51 (s, 9H).

### Step 4; Preparation of 1-(4-amino-3-methylphenyl)-1-(5-trifluoromethylpyridin-2-yl)ethanol

To 0.4 g of t-butyl 4-[1-hydroxy-1-(5-trifluoromethylpyridin-2-yl)ethyl]-2-methylcarbanilate was added dropwise 3.0 ml of trifluoroacetic acid under ice cooling and stirring. After stirring was continued at room temperature for 20 minutes, 50 ml of a saturated aqueous potassium carbonate solution was added to the mixture under ice cooling, the mixture was extracted with 30 ml of chloroform, the organic layer was dried over anhydrous sodium sulfate, and the solvent was removed under reduced pressure to obtain 0.27 g of the objective product as brownish oily substance.

¹H NMR (CDCl₃, Me₄Si, 300 MHz) δ 8.78 (bs, 1H), 7.84 (dd, J=8.3, 2.1 Hz, 1H), 7.43 (d, J=8.3 Hz, 1H), 7.05-7.15 (m, 2H), 6.61 (d, J=8.3 Hz, 1H), 5.15 (s, 1H), 3.59 (bs, 2H), 2.13 (s, 3H), 1.90 (s, 3H).

### Step 5; Preparation of N¹-[4-[1-hydroxy-1-(4-trifluoromethylpyridin-2-yl)ethyl]-2-methylphenyl]-3-iodo-N 2-isopropylphthalic diamide

To 5 ml of a toluene solution containing 0.36 g of 3-iodo-N-isopropylphthalamidic acid was added dropwise 0.26 g of trifluoroacetic anhydride at room temperature under stirring. The mixture was stirred at the same temperature for 2 hours, then, the solvent was removed under reduced pressure, the residue was dissolved in 3.0 ml of acetonitrile, 0.27 g of 1-(4-amino-3-methylphenylyl-(5-trifluoromethylpyridin-2-yl)ethanol was added to the mixture, and stirring was continued at room temperature for 2 hours. After completion of the reaction, the solvent was removed under reduced pressure, and the residue was purified by silica gel chromatography eluting with ethyl acetate-hexane (3:2) to obtain 0.43 g of the objective product as brownish oily substance.

¹H NMR (CDCl₃, Me₄Si, 300 MHz) δ 8.80 (bs, 1H), 8.29 (bs, 1H), 7.7-8.05 (m, 4H), 7.1-7.5 (m, 4H), 5.84 (d, J=8.3 Hz, 1H), 5.28 (s, 1H), 4.1-4.3 (m, 1H), 2.28 (s, 3H), 1.94 (s, 3H), 1.17 (d, J=6.6 Hz, 6H).

### Synthetic Example 2

### N¹-[4-[1-(4-bromodifluoromethoxyphenyl)-2,2,2-trifluoro-1-hydroxyethyl]-2-methylphenyl]-3-iodo-N²-isopropylphthalic diamide (Present Compound No. 1-010)

### Step 1; Preparation of 4′-bromodifluoromethoxy-2,2,2-trifluoroacetophenone

To 10 ml of a 1,3-dimethyl-2-imidazolidinone solution containing 2.0 g of 2,2,2-trifluoro-4′-hydroxyacetophenone was added 0.51 g of 55% oily sodium hydride under ice cooling and stirring, and the mixture was stirred at room temperature for 30 minutes. Then, the reaction mixture was added dropwise to 10 ml of a 1,3-dimethyl-2-imidazolidinone solution containing 11.0 g of dibromodifluoromethane under ice cooling and stirring with a rate in which an innter temperature was maintained at 10° C. or lower. After completion of the dropwise addition, the temperature of the mixture was raised to room temperature, 0.12 g of potassium t-butoxide was added to the mixture, and then, stirring was continued at the same temperature for further 1 hour. After completion of the reaction, the reaction mixture was poured into 150 ml of ice-water, extracted with 100 ml of diethyl ether, the organic layer was washed with water, dried over anhydrous magnesium sulfate, and the solvent was removed under reduced pressure. The residue was purified by silica gel chromatography eluting with ethyl acetate-hexane (1:19 to 1:9) to obtain 1.83 g of the objective product as yellow oily substance.

¹H NMR (CDCl₃, Me₄Si, 300 MHz) δ 8.16 (d, J=8.8 Hz, 2H), 7.43 (d, J=8.8 Hz, 2H).

### Step 2; Preparation of t-butyl 4-[1-(4-bromodifluoromethoxyphenyl)-2,2,2-trifluoro-1-hydroxyethyl]-2-methylcarbanilate

Under nitrogen atmosphere to 25 ml of t-butyl methyl ether solution containing 1.91 g of t-butyl 4-iodo-2-methylcarbanilate was added dropwise 8.0 ml of n-butyl lithium (1.58M hexane solution) at −60° C. under stirring, and after completion of the dropwise addition, the temperature of the mixture was raised to 0° C., and the mixture was stirred for further 30 minutes. Then, the reaction mixture was cooled to −78° C., 7.0 g of 4′-bromodifluoromethoxy-2,2,2-trifluoroacetophenone was added, and the temperature of the mixture was gradually raised to 0° C., and stirring was further continued at the same temperature for 30 minutes. After completion of the reaction, the reaction mixture was poured into 100 ml of a saturated aqueous ammonium chloride solution, the organic layer was collected by separation, and the aqueous layer was extracted with 50 ml of ethyl acetate. The organic layers were combined, dried over anhydrous magnesium sulfate, and the solvent was removed under reduced pressure. The residue was purified by silica gel chromatography eluting with ethyl acetate-hexane (1:19 to 1:4) to obtain 1.0 g of the objective product as brownish oily substance.

¹H NMR (CDCl₃, Me₄Si, 300 MHz) δ 7.87 (d, J=8.3 Hz, 1H), 7.51 (d, J=8.8 Hz, 2H), 7.15-7.35 (m, 4H), 6.32 (bs, 1H), 3.00 (bs, 1H), 2.24 (s, 3H), 1.52 (s, 9H).

### Step 3; Preparation of 1-(4-amino-3-methylphenyl)-1-(4-bromodifluoromethoxyphenyl)-2,2,2-trifluoroethanol

To 1.0 g of t-butyl 4-[1-(4-bromodifluoromethoxyphenyl)-2,2,2-trifluoro-1-hydroxyethyl]-2-methylcarbanilate was added dropwise 3.0 ml of trifluoroacetic acid under ice cooling and stirring. After stirring was continued at room temperature for 20 minutes, under ice cooling, 50 ml of a saturated aqueous potassium carbonate solution was added to the mixture, the mixture was extracted with 50 ml of chloroform, the organic layer was dried over anhydrous sodium sulfate, and the solvent was removed under reduced pressure to obtain 0.6 g of the objective product as brown crystals.

Melting point: 77.5 to 80.5° C.

¹H NMR (CDCl₃, Me₄Si, 300 MHz) δ 7.53 (d, J=8.5 Hz, 2H), 7.0-7.25 (m, 4H), 6.61 (d, J=8.0 Hz, 1H), 2.65-3.15 (bs, 3H), 2.13 (s, 3H).

### Step 4; Preparation of N¹-[4-[1-(4-bromodifluoromethoxyphenyl)-2,2,2-trifluoro-1-hydroxyethyl]-2-methylphenyl]-3-iodo-N²-isopropylphthalic diamide

To 5 ml of a toluene solution containing 0.25 g of 3-iodo-N-isopropylphthalamidic acid was added dropwise 0.18 g of trifluoroacetic anhydride at room temperature under stirring. After the mixture was stirred at the same temperature for 1 hour, the solvent was removed under reduced pressure, the residue was dissolved in 3 ml of acetonitrile, 0.27 g of 1-(4-amino-3-methylphenyl)-1-(4-bromodifluoromethoxyphenyl)-2,2,2-trifluoroethanol was added to the mixture, and stirring was continued at room temperature for 15 hours. After completion of the reaction, the solvent was removed under reduced pressure, and the residue was purified by silica gel chromatography eluting with ethyl acetate-hexane (2:3) to obtain 0.13 g of the objective product as brown crystals.

Melting point: 97.0 to 102.5° C.

¹H NMR (CDCl₃, Me₄Si, 300 MHz) δ 8.39 (bs, 1H), 8.06 (d, J=8.8 Hz, 1H), 7.96 (d, J=7.8 Hz, 1H), 7.78 (d, J=8.0 Hz, 1H), 7.53 (d, J=8.8 Hz, 2H), 7.1-7.35 (m, 5H), 5.86 (d, J=8.0 Hz, 1H), 4.15-4.3 (m, 1H), 3.16 (s, 1H), 2.30 (s, 3H), 1.17 (d, J=6.6 Hz, 6H).

### Synthetic Example 3

### N¹-[4-[2,2,2-trifluoro-1-hydroxy-1-(4-trifluoromethoxyphenyl)ethyl]-2-methylphenyl]-3-iodo-N²-isopropylphthalic diamide (Present Compound No. 1-009)

### Step 1; Preparation of [2,2,2-trifluoro-1-methoxy-1-(4-trifluoromethoxyphenyl)ethyl]-trimethylsilane

To 55 ml of a 1,2-dimethoxyethane solution containing 10.0 g of methyl 4-trifluoromethoxybenzoate were added 13.6 g of (trifluoromethyl)trimethylsilane and 0.1 g of cesium fluoride under ice-cooling and stirring, and the temperature of the mixture was raised to room temperature over 2 hours under stirring. After stirring was continued at room temperature for further 1 hour, the reaction mixture was poured into 200 ml of ice-water, extracted with 200 ml of ethyl acetate, the organic layer was dried over anhydrous magnesium sulfate, and then, the solvent was removed under reduced pressure. The residue was purified by silica gel chromatography eluting with hexane to obtain 16.0 g of the objective product as colorless oily substance.

¹H NMR (CDCl₃, Me₄Si, 300 MHz) δ 7.62 (d, J=8.5 Hz, 2H), 7.23 (d, J=8.5 Hz, 2H), 3.21 (s, 3H), 0.27 (s, 9H).

### Step 2; Preparation of 2,2,2-trifluoro-4′-trifluoromethoxyacetophenone

To 55 ml of a tetrahydrofuran solution containing 16.0 g of [2,2,2-trifluoro-1-methoxy-1-(4-trifluoromethoxyphenyl)ethyl]trimethylsilane was added 9.5 ml of tetrahydrofuran solution containing 1 M of tetrabutyl ammonium fluoride at room temperature under stirring, and the mixture was stirred at room temperature for 20 minutes. After completion of the reaction, to the reaction mixture was added 200 ml of saturated aqueous sodium hydrogen carbonate solution, the reaction mixture was extracted with 200 ml of ethyl acetate, the organic layer was dried over anhydrous magnesium sulfate, and then, the solvent was removed under reduced pressure to obtain 10.8 g of the objective product as brownish oily substance.

¹H NMR (CDCl₃, Me₄Si, 300 MHz) δ 8.15 (d, J=8.8 Hz, 2H), 7.37 (d, J=8.8 Hz, 2H).

### Step 3; Preparation of t-butyl 4-[2,2,2-trifluoro-1-hydroxy-1-(4-trifluoromethoxyphenyl)-ethyl]-2-methylcarbanilate

Under nitrogen atmosphere, to 130 ml of a t-butyl methyl ether solution containing 10.0 g of t-butyl 4-iodo-2-methylcarbanilate was added dropwise 41.8 ml of n-butyl lithium (1.58M hexane solution) at −60° C. under stirring, and after completion of the dropwise addition, the temperature of the mixture was raised to 0° C., and the mixture was stirred for further 30 minutes. Then, the reaction mixture was cooled to −78° C., 7.75 g of 2,2,2-trifluoro-4′-trifluoromethoxyacetophenone was added to the mixture, the temperature of the mixture was gradually raised to 0° C., and stirring of the mixture was continued at the same temperature for further 30 minutes. After completion of the reaction, the reaction mixture was poured into 300 ml of a saturated aqueous ammonium chloride solution, the organic layer was collected by separation, and the aqueous layer was extracted with 100 ml of ethyl acetate. The organic layers were combined, dried over anhydrous magnesium sulfate, and the solvent was removed under reduced pressure. The residue was purified by silica gel chromatography eluting with ethyl acetate-hexane (1:19 to 1:4) to obtain 9.0 g of the objective product as colorless oily substance.

¹H NMR (CDCl₃, Me₄Si, 300 MHz) δ 7.87 (d, J=8.5 Hz, 1H), 7.50 (d, J=8.8 Hz, 2H), 7.1-7.35 (m, 4H), 6.31 (bs, 1H), 2.95 (bs, 1H), 2.23 (s, 3H), 1.52 (s, 9H).

### Step 4; Preparation of 1-(4-amino-3-methylphenyl)-2,2,2-trifluoro-1-(4-trifluoromethoxyphenyl)ethanol

To 9.0 g of t-butyl 4-[2,2,2-trifluoro-1-hydroxy-1-(4-trifluoromethoxyphenyl)ethyl]-2-methylcarbanilate was added dropwise 16.0 ml of trifluoroacetic acid under ice cooling and stirring. After stirring was continued at room temperature for 30 minutes, 200 ml of a saturated aqueous potassium carbonate solution was added to the mixture under ice cooling, the mixture was extracted with 100 ml of chloroform and 100 ml of ethyl acetate, the organic layers were combined, dried over anhydrous sodium sulfate, and the solvent was removed under reduced pressure to obtain 6.8 g of the objective product as yellow crystals.

Melting point: 127.0 to 128.5° C.

¹H NMR (CDCl₃, Me₄Si, 300 MHz) δ 7.51 (d, J=8.5 Hz, 2H), 7.0-7.25 (m, 4H), 6.60 (d, J=8.3 Hz, 1H), 3.06 (bs, 3H), 2.13 (s, 3H).

### Step 5; Preparation of N¹-[4-[2,2,2-trifluoro-1-hydroxy-1-(4-trifluoromethoxyphenyl)ethyl]-2-methylphenyl]-3-iodo-N²-isopropylphthalic diamide

To 5 ml of a toluene solution containing 0.2 g of 3-iodo-N-isopropylphthalamidic acid was added dropwise 1.0 g of trifluoroacetic anhydride at room temperature under stirring. After stirring the mixture at the same temperature for 30 minutes, the solvent was removed under reduced pressure, the residue was dissolved in 5 ml of acetonitrile, 0.2 g of 1-(4-amino-3-methylphenyl)-2,2,2-trifluoro-1-(4-trifluoromethoxyphenyl)ethanol and one drop of trifluoroacetic acid were added to the mixture, and stirring was continued at room temperature for 4 hours. After completion of the reaction, the solvent was removed under reduced pressure, and the residue was purified by silica gel chromatography eluting with ethyl acetate-hexane (1:1) to obtain 0.33 g of the objective product as colorless resinous substance.

¹H NMR (CDCl₃, Me₄Si, 400 MHz) δ 8.39 (bs, 1H), 8.10 (d, J=8.8 Hz, 1H), 7.95 (d, J=7.6 Hz, 1H), 7.80 (d, J=8.0 Hz, 1H), 7.52 (d, J=8.0 Hz, 2H), 7.2-7.35 (m, 5H), 5.90 (d, J=8.0 Hz, 1H), 4.15-4.3 (m, 1H), 3.01 (s, 1H), 2.33 (s, 3H), 1.17 (d, J=6.6 Hz, 6H).

### Synthetic Example 4

### N¹-[4-[2,2,2-trifluoro-1-hydroxy-1-(2,2,3,3-tetrafluoro-1,4-dioxa-2,3-dihydronaphthalen-6-yl)ethyl]-2-methylphenyl]-3-iodo-N²-isopropylphthalic diamide (Present compound No. 1-028)

### Step 1; Preparation of t-butyl 2-methyl-4-trifluoroacetylcarbanilate

Under nitrogen atmosphere, to 300 ml of a diethyl ether solution containing 10.0 g of t-butyl 4-iodo-2-methylcarbanilate was added dropwise 45.0 ml of n-butyl lithium (1.5M hexane solution) at −10° C. under stirring, and after completion of the dropwise addition, the mixture was stirred at the same temperature for 30 minutes. Then, the reaction mixture was cooled to −78° C., 9.5 g of trifluoroethyl acetate was added dropwise to the mixture, and after completion of the dropwise addition, stirring was continued at the same temperature for further 1 hour. After completion of the reaction, the temperature of the reaction mixture was raised to −10° C., 100 ml of 2N hydrochloric acid was added and the mixture was vigorously stirred, then, the organic layer was collected by separation, dehydrated and dried by saturated saline solution and then anhydrous magnesium sulfate in this order, and the solvent was removed under reduced pressure. The residue was purified by silica gel chromatography eluting with diethyl ether-hexane (1:4) to obtain 3.2 g of the objective product as white crystals.

Melting point: 85.0 to 87.0° C.

¹H NMR (CDCl₃, Me₄Si, 300 MHz) δ 8.25 (d, J=8.8 Hz, 1H), 7.8-8.0 (m, 2H), 6.62 (bs, 1H), 2.32 (s, 3H), 1.55 (s, 9H).

### Step 2; Preparation of t-butyl 4-[2,2,2-trifluoro-1-hydroxy-1-(2,2,3,3-tetrafluoro-1,4-dioxa -2,3-dihydronaphthalen-6-yl)ethyl]-2-methylcarbanilate

Under nitrogen atmosphere, to 30 ml of a t-butylmethylether solution containing 2.37 g of 6-bromo-2,2,3,3-tetrafluoro-1,4-dioxa-2,3-dihydronaphthalene was added dropwise 5.4 ml of n-butyl lithium (1.58M hexane solution) at −40° C. under stirring, and after completion of the dropwise addition, the temperature of the mixture was raised to 0° C., and the mixture was stirred for further 1 hour. Then, the reaction mixture was cooled to −50° C., 20 ml of a t-butylmethylether solution containing 1.0 g of t-butyl 2-methyl-4-trifluoroacetylcarbanilate was added dropwise to the mixture, the temperature of the mixture was gradually raised to 0° C., and stirring was continued at the same temperature for further 1 hour. After completion of the reaction, the reaction mixture was poured into 100 ml of a saturated aqueous ammonium chloride solution, the organic layer was collected by separation, and the aqueous layer was extracted with 50 ml of ethyl acetate. The organic layers were combined, dehydrated and dried by saturated saline solution and then anhydrous magnesium sulfate in this order, and the solvent was removed under reduced pressure. The residue was purified by silica gel chromatography eluting with ethyl acetate-hexane (1:4) to obtain 2.3 g of the objective product as yellow oily substance.

### Step 3; Preparation of 1-(4-amino-3-methylphenyl)-2,2,2-trifluoro-1-(2,2,3,3-tetrafluoro -1,4-dioxa-2,3-dihydronaphthalen-6-yl)ethanol

To 2.3 g of t-butyl 4-[2,2,2-trifluoro-1-hydroxy-1-(2,2,3,3-tetrafluoro-1,4-dioxa -2,3-dihydronaphthalen-6-yl)ethyl]-2-methylcarbanilate was added dropwise 15.0 ml of trifluoroacetic acid under ice cooling and stirring. After stirring was continued at room temperature for 30 minutes, excess trifluoroacetic acid was removed under reduced pressure, the residue was dissolved in 100 ml of ethyl acetate, washed with 100 ml of saturated aqueous sodium carbonate solution, dehydrated and dried by saturated saline solution and then anhydrous magnesium sulfate in this order, and the solvent was removed under reduced pressure. The residue was purified by silica gel chromatography eluting with ethyl acetate-hexane (2:3) and high performance liquid chromatography eluting with acetonitrile-water (80:20) to obtain 0.5 g of the objective product as white crystals.

Melting point: 147.5 to 150.0° C.

¹H NMR (CDCl₃, Me₄Si, 300 MHz) δ 7.33 (s, 1H), 7.24 (d, J=9.9 Hz, 1H), 7.05-7.1 (m, 3H), 6.58 (d, J=9.1 Hz, 1H), 3.72 (bs, 2H), 3.45 (bs, 1H), 2.12 (s, 3H).

### Step 4; Preparation of N¹-[4-[2,2,2-trifluoro-1-hydroxy-1-(2,2,3,3-tetrafluoro-1,4-dioxa -2,3-dihydronaphthalen-6-yl)ethyl]-2-methylphenyl]-3-iodo-N²-isopropylphthalic diamide

To 10 ml of a toluene solution containing 0.15 g of 3-iodo-N-isopropylphthalamidic acid was added dropwise 0.15 g of trifluoroacetic anhydride at room temperature under stirring. After stirring was continued at the same temperature for 30 minutes, the solvent was removed under reduced pressure, the residue was dissolved in 3 ml of acetonitrile, 5 ml of an acetonitrile solution containing 0.15 g of 1-(4-amino-3-methylphenyl) -2,2,2-trifluoro-1-(2,2,3,3-tetrafluoro-1,4-dioxa-2,3-dihydronaphthalen-6-yl)ethanol was added dropwise to the mixture, and after completion of the dropwise addition, stirring was continued at room temperature for 1 hour. After completion of the reaction, the solvent was removed under reduced pressure, the residue was purified by silica gel chromatography eluting with ethyl acetate-hexane (2:3) to obtain 0.25 g of the objective product as colorless resinous substance.

¹H NMR (CDCl₃, Me₄Si, 300 MHz) δ 8.42 (s, 1H), 8.10 (d, J=8.8 Hz, 1H), 7.9-8.05 (m, 2H), 7.73 (d, J=7.8 Hz, 1H), 7.05-7.35 (m, 6H), 5.98 (d, J=8.1 Hz, 1H), 4.1-4.25 (m, 1H), 3.69 (s, 1H), 2.28 (s, 3H), 1.16 (d, J=6.6 Hz, 6H).

### Synthetic Example 5

### N¹-[4-[2,2,2-trifluoro-1-hydroxy-1-(4-methanesulfonyloxyphenyl)ethyl]-2-methylphenyl]-3-iodo-N²-isopropylphthalic diamide (Present Compound No. 1-015)

### Step 1; Preparation of 4′-hydroxy-3-methyl-4-nitrobenzophenone

To 30 ml of a dichloromethane solution containing 2.0 g of 4′-methoxy-3-methyl -4-nitrobenzophenone was added 2.1 ml of boron tribromide under ice cooling and stirring, and the mixture was stirred at room temperature for 20 hours. After completion of the reaction, 2 ml of diethyl ether was added to the reaction mixture, and the resulting mixture was stirred for 10 minutes. Then, the mixture was poured into 30 ml of water, the organic layer was collected by separation, washed with water, dehydrated and dried by saturated saline solution and then anhydrous sodium sulfate in this order, and the solvent was removed under reduced pressure. The remained solid was washed with a mixed solvent of diisopropyl ether-hexane to obtain 1.8 g of the objective product as pale purple crystals.

Melting point: 146.0 to 148.0° C.

¹H NMR (CDCl₃-DMSO-d₆, Me₄Si, 300 MHz) δ 9.71 (bs, 1H), 8.02 (d, J=8.3 Hz, 1H), 7.55-7.85 (m, 4H), 6.94 (d, J=8.3 Hz, 2H), 2.68 (s, 3H).

### Step 2; Preparation of 4-(3-methyl-4-nitrobenzoyl)phenyl methanesulfonate

To 5 ml of an N,N-dimethylformamide solution containing 0.50 g of 4′-hydroxy -3-methyl-4-nitrobenzophenone and 0.20 g of triethylamine was added 0.22 g of methanesulfonyl chloride under ice cooling and stirring, and the mixture was stirred at the same temperature for 20 minutes. After completion of the reaction, the reaction mixture was poured into 30 ml of water, extracted with 50 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (2:5) to obtain 0.54 g of the objective product as brownish oily substance.

¹H NMR (CDCl₃, Me₄Si, 300 MHz) δ 8.05 (d, J=8.3 Hz, 1H), 7.89 (d, J=8.8 Hz, 2H), 7.75 (s, 1H), 7.72 (d, J=8.3 Hz, 1H), 7.45 (d, J=8.8 Hz, 2H), 3.25 (s, 3H), 2.66 (s, 3H).

### Step 3; Preparation of 4-[1-(4-amino-3-methylphenyl)-2,2,2-trifluoro-1-hydroxyethyl]-phenyl methanesulfonate

To 3 ml of a 1,2-dimethoxyethane solution containing 0.54 g of 4-(3-methyl-4-nitrobenzoyl)phenyl methanesulfonate and 1.5 ml of (trifluoromethyl)trimethylsilane was added 0.06 g of cesium fluoride under ice cooling and stirring, and then, the temperature of the mixture was raised to room temperature, and stirring was continued for further 18 hours. After completion of the reaction, the reaction mixture was poured into 30 ml of water, extracted with 50 ml of diethyl ether, the organic layer was dehydrated and dried by saturated saline solution and then anhydrous magnesium sulfate in this order, and the solvent was removed under reduced pressure. The residue was dissolved in 3 ml of tetrahydrofuran, 0.3 ml of a tetrahydrofuran solution containing 1 M of tetrabutyl ammonium fluoride was added to the mixture at room temperature under stirring, and the resulting mixture was stirred at room temperature for 10 minutes. After completion of the reaction, the solvent was removed under reduced pressure, 30 ml of water was added to the residue, the mixture was extracted with 30 ml of ethyl acetate, dehydrated and dried by saturated saline solution and then anhydrous magnesium sulfate in this order, and the dryer was removed by filtration. To the filtrate was added 0.2 g of 5% palladium-carbon, and the mixture was stirred under hydrogen atmosphere at room temperature for 20 hours. After completion of the reaction, palladium-carbon was filtered off with Celite, the solvent was removed under reduced pressure, and the residue was purified by silica gel chromatography eluting with ethyl acetate-hexane (1:2 to 2:3) to obtain 0.24 g of the objective product as white crystals.

Melting point: 137.0 to 139.0° C.

¹H NMR (CDCl₃, Me₄Si, 300 MHz) δ 7.54 (d, J=8.8 Hz, 2H), 7.25 (d, J=7.7 Hz, 2H), 7.11 (s, 1H), 7.09 (d, J=8.3 Hz, 1H), 6.62 (d, J=9.0 Hz, 1H), 3.72 (bs, 2H), 3.16 (s, 3H), 2.94 (bs, 1H), 2.14 (s, 3H).

### Step 4; Preparation of N¹-[4-[2,2,2-trifluoro-1-hydroxy-1-(4-methanesulfonyloxyphenyl)-ethyl]-2-methylphenyl]-3-iodo-N²-isopropylphthalic diamide

To 5 ml of a toluene solution containing 0.10 g of 3-iodo-N-isopropylphthalamidic acid was added dropwise 0.09 g of trifluoroacetic anhydride at room temperature under stirring. After stirring at the same temperature for 30 minutes, the solvent was removed under reduced pressure, the residue was dissolved in 4 ml of acetonitrile, 0.11 g of 4-[1-(4-amino-3-methylphenyl)-2,2,2-trifluoro-1-hydroxyethyl]phenyl methanesulfonate and 1 drop of trifluoroacetic acid were added to the mixture, and the resulting mixture was stirred at room temperature for 18 hours. After completion of the reaction, the solvent was removed under reduced pressure, and the residue was purified by silica gel chromatography eluting with ethyl acetate-hexane (1:1) to obtain 0.20 g of the objective product as colorless resinous solid.

Melting point: 111.0 to 114.0° C.

¹H NMR (CDCl₃, Me₄Si, 300 MHz) δ 8.39 (bs, 1H), 8.08 (d, J=9.1 Hz, 1H), 7.97 (d, J=8.0 Hz, 1H), 7.79 (d, J=7.1 Hz, 1H), 7.54 (d, J=8.8 Hz, 2H), 7.15-7.4 (m, 5H), 5.17 (d, J=8.0 Hz, 1H), 4.15-4.3 (m, 1H), 3.17 (s, 3H), 3.07 (s, 1H), 2.30 (s, 3H), 1.17 (d, J=6.6 Hz, 6H).

### Synthetic Example 6

### N¹-[4-[3-(4-chlorophenyl)-1-hydroxy-1-trifluoromethyl-2-propenyl]-2-methylphenyl]-3-iodo-N²-isopropylphthalic diamide (Present Compound No. 2-018)

### Step 1; Preparation of t-butyl 4-[3-(4-chlorophenyl)-1-hydroxy-1-trifluoromethyl-2-propenyl]-2-methylcarbanilate

Under nitrogen atmosphere, to 140 ml of a t-butylmethylether solution containing 10.0 g of t-butyl 4-iodo-2-methylcarbanilate was added dropwise 41.8 ml of n-butyl lithium (1.58M hexane solution) at −60° C. under stirring, and after completion of the dropwise addition, the temperature of the mixture was raised to 0° C., and the mixture was stirred for further 30 minutes. Then, the reaction mixture was cooled to −78° C., 7.0 g of 4-(4-chlorophenyl)-1,1,1-trifluoro-3-buten-2-one was added, and the temperature of the mixture was gradually raised to 0° C., and stirring was continued at the same temperature for further 30 minutes. After completion of the reaction, the reaction mixture was poured into 300 ml of a saturated aqueous ammonium chloride solution, the organic layer was collected by separation, and the aqueous layer was extracted with 100 ml of ethyl acetate. The organic layers were combined, dried over anhydrous magnesium sulfate, and the solvent was removed under reduced pressure. The residue was purified by silica gel chromatography eluting with ethyl acetate-hexane (1:9 to 2:3) and alumina column chromatography eluting with chloroform to obtain 4.2 g of the objective product as colorless oily substance.

¹H NMR (CDCl₃, Me₄Si, 300 MHz) δ 7.90 (d, J=8.1 Hz, 1H), 7.25-7.5 (m, 6H), 6.82 (d, J=16.0 Hz, 1H), 6.63 (d, J=16.0 Hz, 1H), 6.31 (bs, 1H), 2.73 (s, 1H), 2.27 (s, 3H), 1.52 (s, 9H).

### Step 2; Preparation of 1-(4-amino-3-methylphenyl)-3-(4-chlorophenyl)-1-trifluoromethyl-2-propen-1-ol

To 4.2 g of t-butyl 4-[3-(4-chlorophenyl)-1-hydroxy-1-trifluoromethyl-2-propenyl]-2-methylcarbanilate was added dropwise 8.0 ml of trifluoroacetic acid under ice cooling and stirring. After stirring was continued at room temperature for 30 minutes, under ice cooling, 150 ml of a saturated aqueous potassium carbonate solution was added to the mixture, the resulting mixture was extracted with 100 ml of chloroform, the organic layer was dried over anhydrous sodium sulfate, and the solvent was removed under reduced pressure. The remained solid was washed with 10 ml of diisopropyl ether to obtain 2.4 g of the objective product as white crystals.

Melting point: 126.5 to 129.0° C.

¹H NMR (CDCl₃, Me₄Si, 300 MHz) δ 7.15-7.4 (m, 6H), 6.83 (d, J=16.0 Hz, 1H), 6.55-6.75 (m, 2H), 2.8-3.5 (broad, 3H), 2.17 (s, 3H).

### Step 3; Preparation of N¹-[4-[3-(4-chlorophenyl)-1-hydroxy-1-trifluoromethyl-2-propenyl]-2-methylphenyl]-3-iodo-N²-isopropylphthalic diamide

To 20 ml of a toluene solution containing 1.17 g of 3-iodo-N-isopropylphthalamidic acid was added dropwise 0.86 g of trifluoroacetic anhydride at room temperature under stirring. After the mixture was stirred at the same temperature for 1 hour, the solvent was removed under reduced pressure, the residue was dissolved in 10 ml of acetonitrile, 1.0 g of 1-(4-amino-3-methylphenyl)-3-(4-chlorophenyl)-1-trifluoromethyl-2-propen-1-ol was added to the solution, and stirring of the mixture was continued at room temperature for 3 hours. After completion of the reaction, the solvent was removed under reduced pressure, the residue was purified by silica gel chromatography eluting with ethyl acetate-hexane (1:9 to 2:3) to obtain 1.50 g of the objective product as white crystals.

Melting point: 108.0 to 113.0° C.

¹H NMR (CDCl₃, Me₄Si, 400 MHz) δ 8.38 (s, 1H), 8.07 (d, J=8.8 Hz, 1H), 7.96 (d, J=8.0 Hz, 1H), 7.78 (d, J=8.0 Hz, 1H), 7.15-7.5 (m, 7H), 6.83 (d, J=16.0 Hz, 1H), 6.64 (d, J=16.0 Hz, 1H), 5.85 (d, J=8.4 Hz, 1H), 4.15-4.3 (m, 1H), 2.99 (s, 1H), 2.34 (s, 3H), 1.16 (d, J=6.4 Hz, 6H).

### Synthetic Example 7

### N¹-[4-[1-(4-chlorophenyl)-2,2,2-trifluoro-1-methoxyethyl]-2-methylphenyl]-3-iodo-N²-isopropylphthalic diamide (Present Compound No. 3-002)

### Step 1; Preparation of t-butyl 4-[1-(4-chlorophenyl)-2,2,2-trifluoro-1-hydroxyethyl]-2-methylcarbanilate

Under nitrogen atmosphere, to 40 ml of a t-butylmethylether solution containing 3.0 g of t-butyl 4-iodo-2-methylcarbanilate was added dropwise 12.5 ml of n-butyl lithium (1.58M hexane solution) at −50° C. under stirring, and after completion of the dropwise addition, the temperature of the mixture was raised to 0° C., and the mixture was stirred for further 30 minutes. Then, the reaction mixture was cooled to −78° C., 1.88 g of 4′-chloro-2,2,2-trifluoroacetophenone was added to the mixture, the temperature of the mixture was gradually raised to 0° C., and stirring was continued at the same temperature for further 30 minutes. After completion of the reaction, to the reaction mixture was added 100 ml of a saturated aqueous ammonium chloride solution, the organic layer was collected by separation, and the aqueous layer was extracted with 100 ml of ethyl acetate. The organic layers were combined, dried over anhydrous magnesium sulfate, and the solvent was removed under reduced pressure. The residue was purified by silica gel chromatography eluting with ethyl acetate-hexane (1:9 to 2:3) to obtain 2.96 g of the objective product as colorless transparent oily substance.

¹H NMR (CDCl₃, Me₄Si, 300 MHz) δ 7.86 (d, J=8.7 Hz, 1H), 7.40 (d, J=8.4 Hz, 2H), 7.15-7.35 (m, 4H), 6.30 (bs, 1H), 2.93 (s, 1H), 2.22 (s, 3H), 1.52 (s, 9H).

### Step 2; Preparation of 1-(4-amino-3-methylphenyl)-1-(4-chlorophenyl)-2,2,2-trifluoroethanol

To 2.96 g of t-butyl 4-[1-(4-chlorophenyl)-2,2,2-trifluoro-1-hydroxyethyl]-2-methylcarbanilate was added dropwise 6.0 ml of trifluoroacetic acid under ice cooling and stirring. After stirring was continued at room temperature for 20 minutes, 70 ml of a saturated aqueous potassium carbonate solution was added to the mixture under ice cooling, the mixture was extracted with 100 ml of chloroform, the organic layer was dried over anhydrous sodium sulfate, and the solvent was removed under reduced pressure to obtain 1.80 g of the objective product as brown solid.

Melting point: 157.0 to 160.5° C.

¹H NMR (CDCl₃, Me₄Si, 300 MHz) δ 7.45 (d, J=8.8 Hz, 2H), 7.28 (d, J=8.8 Hz, 2H), 7.05-7.15 (m, 2H), 6.60(d, J=8.3 Hz, 1H), 5.03 (bs, 1H), 3.74 (bs, 2H), 2.12 (s, 3H).

### Step 3; Preparation of 4-[1-(4-chlorophenyl)-2,2,2-trifluoro-1-methoxyethyl]-2-methylaniline

To 3 ml of an N,N-dimethylformamide solution containing 0.5 g of 1-(4-amino-3-methylphenyl)-1-(4-chlorophenyl)-2,2,2-trifluoroethanol was added 0.073 g of 55% oily sodium hydride under ice cooling and stirring, the mixture was stirred at room temperature for 20 minutes, then, 0.24 g of methyl iodide was added to the mixture, and stirring was continued at the same temperature for further 2 hours. After completion of the reaction, the reaction mixture was poured into 20 ml of ice-water, extracted with 30 ml of ethyl acetate, the organic layer was dried over anhydrous magnesium sulfate, and then, the solvent was removed under reduced pressure. The residue was purified by silica gel chromatography eluting with ethyl acetate-hexane (2:3) to obtain 0.44 g of the objective product as brownish oily substance.

¹H NMR (CDCl₃, Me₄Si, 300 MHz) δ 7.2-7.4 (m, 4H), 6.9-7.1 (m, 3H), 6.62 (d, J=8.3 Hz, 1H), 3.71 (bs, 2H), 3.27 (s, 3H), 2.14 (s, 3H).

### Step 4; Preparation of N¹-[4-[1-(4-chlorophenyl)-2,2,2-trifluoro-1-methoxyethyl]-2-methylphenyl]-3-iodo-N²-isopropylphthalic diamide

To 5 ml of a toluene solution containing 0.25 g of 3-iodo-N-isopropylphthalamidic acid was added dropwise 0.18 g of trifluoroacetic anhydride at room temperature under stirring. After the mixture was stirred at the same temperature for 1 hour, the solvent was removed under reduced pressure, the residue was dissolved in 3.0 ml of acetonitrile, 0.21 g of 4-[1-(4-chlorophenyl)-2,2,2-trifluoro-1-methoxyethyl]-2-methylaniline was added to the mixture, and stirring was continued at room temperature for 14 hours. After completion of the reaction, precipitated crystals were collected by filtration and washed with a small amount of acetonitrile to obtain 0.2 g of the objective product as white crystals.

Melting point: 211.5 to 214.0° C.

¹H NMR (CDCl₃, Me₄Si, 300 MHz) δ 8.35 (bs, 1H), 8.09 (d, J=8.8 Hz, 1H), 7.97 (d, J=7.6 Hz, 1H), 7.79 (d, J=8.0 Hz, 1H), 7.15-7.35 (m, 7H), 5.83 (d, J=8.0 Hz, 1H), 4.15-4.3 (m, 1H), 3.30 (s, 3H), 2.31 (s, 3H), 1.17 (d, J=6.6 Hz, 6H).

### Synthetic Example 8

### N¹-[4-[2-(4-chlorophenoxy)-1-hydroxy-1-trifluoromethylethyl]-2-methylphenyl]-3-iodo-N²-isopropylphthalic diamide (Present Compound No. 2-004)

### Step 1; Preparation of t-butyl 4-[2-(4-chlorophenoxy)-1-hydroxy-1-trifluoromethylethyl]-2-methylcarbanilate

To 30 ml of an N,N-dimethylformamide solution containing 2.0 g of t-butyl 2-methyl-4-(1-trifluoromethyloxirane-1-yl)carbanilate and 1.5 g of 4-chlorophenol was added 0.45 g of 60% oily sodium hydride under ice cooling and stirring, and the mixture was stirred at room temperature for 24 hours. After completion of the reaction, the reaction mixture was poured into 150 ml of ice-water and extracted with 100 ml of diethyl ether, and the organic layer was dehydrated and dried by a saturated saline solution and then anhydrous magnesium sulfate in this order, and the solvent was removed under reduced pressure. The residue was purified by silica gel chromatography eluting with ethyl acetate-hexane (1:4) to obtain 2.1 g of the objective product as colorless oily substance.

### Step 2; Preparation of N¹-[4-[2-(4-chlorophenoxy)-1-hydroxy-1-trifluoromethylethyl]-2-methylphenyl]-3-iodo-N²-isopropylphthalic diamide

To 1.0 g of t-butyl 4-[2-(4-chlorophenoxy)-1-hydroxy-1-trifluoromethylethyl]-2-methylcarbanilate was added dropwise 5.0 ml of trifluoroacetic acid under ice cooling and stirring. Stirring was continued at room temperature for 10 minutes, then, excess trifluoroacetic acid was removed under reduced pressure to obtain crude 1-(4-amino-3-methylphenyl)-2-(4-chlorophenoxy)-1-trifluoromethylethanol.

To 10 ml of a toluene solution containing 0.8 g of 3-iodo-N-isopropylphthalamidic acid was added dropwise 0.8 g of trifluoroacetic anhydride at room temperature under stirring. After stirring at the same temperature for 30 minutes, the solvent was removed under reduced pressure, the residue was dissolved in 20 ml of acetonitrile, and the above-mentioned crude 1-(4-amino-3-methylphenyl)-2-(4-chlorophenoxy)-1-trifluoromethylethanol was added thereto, and stirring of the mixture was continued at room temperature for 3 hours. After completion of the reaction, the solvent was removed under reduced pressure, the residue was purified by silica gel chromatography eluting with ethyl acetate-chloroform (2:3) to obtain 0.7 g of the objective product as white crystals.

Melting point: 90.0 to 95.0° C.

### Synthetic Example 9

### N¹-[4-[2-(4-chlorophenoxy)-2,2-difluoro-1-hydroxy-1-trifluoromethylethyl]-2-methylphenyl]-3-iodo-N²-isopropylphthalic diamide (Present Compound No. 2-008)

### Step 1; Preparation of 1-(2-bromo-1,1,2,3,3,3-hexafluoropropyloxy)-4-chlorobenzene

To 100 ml of an N,N-dimethylformamide solution containing 20.0 g of 2,2-dibromo-1,1,2,3,3,3-hexafluoropropane and 7.0 g of 4-chlorophenol was added 2.2 g of 60% oily sodium hydride under ice cooling and stirring, and the mixture was stirred at room temperature for 12 hours. After completion of the reaction, the reaction mixture was poured into 300 ml of ice-water, extracted with 150 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (1:4) to obtain 18.0 g of the objective product as colorless oily substance.

### Step 2; Preparation of 4-[2-(4-chlorophenoxy)-1,2,2-trifluoro-1-trifluoromethylethyl]-2-methylaniline

To 50 ml of a dimethylsulfoxide solution containing 5.0 g of 1-(2-bromo -1,1,2,3,3,3-hexafluoropropyloxy)-4-chlorobenzene and 1.5 g of o-toluidine were added 1.8 g of sodium hydrogen carbonate and 3.8 g of sodium dithionite under stirring, and the mixture was stirred at 70° C. for 3 hours. After completion of the reaction, the reaction mixture was poured into 300 ml of ice-water, extracted with 150 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (1:4) to obtain 3.8 g of the objective product as colorless oily substance.

### Step 3; Preparation of 1-(4-amino-3-methylphenyl)-2-(4-chlorophenoxy)-2,2-difluoro-1-trifluoromethylethanol

To 20 ml of a 1,4-dioxane solution containing 2.0 g of 4-[2-(4-chlorophenoxy) -1,2,2-trifluoro-1-trifluoromethylethyl]-2-methylaniline was added 3.0 g of potassium hydroxide, and the mixture was stirred under reflux for 2 hours. After completion of the reaction, the solvent was removed under reduced pressure, the residue was poured into 100 ml of ice-water and extracted with 100 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (2:3) to obtain 0.6 g of the objective product as pale yellowish oily substance.

### Step 4; Preparation of N¹-[4-[3-(4-chlorophenyl)-1-hydroxy-1-trifluoromethyl-2-propenyl]-2-methylphenyl]-3-iodo-N²-isopropylphthalic diamide

To 5 ml of a toluene solution containing 0.6 g of 3-iodo-N-isopropylphthalamidic acid was added dropwise 0.6 g of trifluoroacetic anhydride at room temperature under stirring. After the mixture was stirred at the same temperature for 30 minutes, the solvent was removed under reduced pressure, the residue was dissolved in 10 ml of acetonitrile, 0.5 g of 1-(4-amino-3-methylphenyl)-2-(4-chlorophenoxy)-2,2-difluoro-1-trifluoromethylethanol was added to the mixture, and stirring was continued at room temperature for 5 hours. After completion of the reaction, the solvent was removed under reduced pressure, and the residue was purified by silica gel chromatography eluting with ethyl acetate-chloroform (2:3) to obtain 0.3 g of the objective product as colorless crystals.

Melting point: 65.0 to 75.0° C.

### Synthetic Example 10

### N¹-(2,2-difluoro-3-hydroxy-5-methyl-3-trifluoromethyl-2,3-dihydrobenzofuran-6-yl)-3-iodo-N²-isopropylphthalic diamide (Present Compound No. 4-002)

### Step 1; Preparation of 1-(2-bromo-1,1,2,3,3,3-hexafluoropropyloxy)-4-methyl-3-nitrobenzene

To 100 ml of an N,N-dimethylformamide solution containing 10.0 g of 2,2-dibromo-1,1,2,3,3,3-hexafluoropropane and 5.0 g of 4-methyl-3-nitrophenol was added 1.4 g of 60% oily sodium hydride under ice cooling and stirring, and the mixture was stirred at room temperature for 12 hours. After completion of the reaction, the reaction mixture was poured into 300 ml of ice-water, extracted with 150 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (1:4) to obtain 8.0 g of the objective product as pale yellowish oily substance.

### Step 2; Preparation of 5-(2-bromo-1,1,2,3,3,3-hexafluoropropyloxy)-2-methylaniline

To 100 ml of an ethanol solution containing 8.0 g of 1-(2-bromo-1,1,2,3,3,3-hexafluoropropyloxy)-4-methyl-3-nitrobenzene were added 9.5 g of tin chloride dehydrate and 15 ml of conc. hydrochloric acid at room temperature under stirring, and the mixture was stirred at the same temperature for 12 hours. After completion of the reaction, the solvent was removed under reduced pressure, the residue was poured into 100 ml of 2N aqueous sodium hydroxide solution, extracted with 100 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (1:4) to obtain 6.0 g of the objective product as pale yellowish oily substance.

### Step 3; Preparation of 6-amino-2,2,3-trifluoro-5-methyl-3-trifluoromethyl-2,3-dihydrobenzofuran

To 50 ml of a dimethylsulfoxide solution containing 6.0 g of 5-(2-bromo-1,1,2,3,3,3-hexafluoropropyloxy)-2-methylaniline were added 2.2 g of sodium hydrogen carbonate and 4.5 g of sodium dithionite under stirring, and the mixture was stirred at 70° C. for 3 hours. After completion of the reaction, the reaction mixture was poured into 300 ml of ice-water and extracted with 150 ml of diethyl ether, the organic layer was dehydrated and dried by saturated saline solution and then anhydrous magnesium sulfate in this order, and the solvent was removed under reduced pressure. The residue was purified by silica gel chromatography eluting with ethyl acetate-hexane (3:7) to obtain 3.0 g of the objective product as colorless oily substance.

### Step 4; Preparation of 6-amino-2,2-difluoro-3-hydroxy-5-methyl-3-trifluoromethyl-2,3-dihydrobenzofuran

1.0 g of 6-amino-2,2,3-trifluoro-5-methyl-3-trifluoromethyl-2,3-dihydrobenzofuran was treated by alumina column chromatography eluting with ethyl acetate-methanol (7:3) to obtain 0.6 g of the objective product as white crystals.

### Step 5; Preparation of N¹-(2,2-difluoro-3-hydroxy-5-methyl-3-trifluoromethyl-2,3-dihydrobenzofuran-6-yl)-3-iodo-N²-isopropylphthalic diamide

To 5 ml of a toluene solution containing 0.6 g of 3-iodo-N-isopropylphthalamidic acid was added dropwise 0.6 g of trifluoroacetic anhydride at room temperature under stirring. After the mixture was stirred at the same temperature for 30 minutes, the solvent was removed under reduced pressure, the residue was dissolved in 10 ml of acetonitrile, 0.5 g of 6-amino-2,2-difluoro-3-hydroxy-5-methyl-3-trifluoromethyl-2,3-dihydrobenzofuran was added to the mixture, and stirring was continued at room temperature for 5 hours. After completion of the reaction, the solvent was removed under reduced pressure, and the residue was purified by silica gel chromatography eluting with ethyl acetate-chloroform (1:1) to obtain 0.4 g of the objective product as colorless crystals.

Melting point: 144.0 to 146.0° C.

Present compounds can be prepared according to the above-mentioned Preparation method and Examples. Examples of such compounds are shown in Table 6 to Table 10, but the present invention is not limited by these.

Incidentally, in the Tables, i-Pr means an isopropyl group, and in the same manner, s-Bu means a secondary butyl group, t-Bu means a tertiary butyl group, Ph means a phenyl group, and 2-Naph means a 2-naphthyl group, respectively,

in the Tables, aromatic heterocyclic rings represented by L-15b to L-45e have the following structures, respectively.

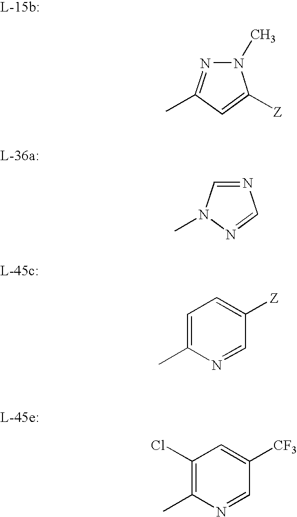

For example, the expression [(D-15b)CF₃] means 1-methyl-5-trifluoromethylpyrazol-3-yl group, and the expression [CH₂(L-36a)] means 1,2,4-triazol-1-yl methyl group.

Also, in the tables, *1 means “resinous state”, and *2 means “oily state”, respectively.

TABLE 6

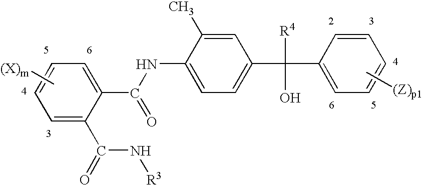

| No. | (X)ₘ | R³ | R⁴ | (Z)ₚ₁ | m.p. (° C.) |
|---|---|---|---|---|---|
| 1-001 | 3-I | i-Pr | CH₃ | 4-Cl | 177.0-179.0 |
| 1-002 | 3-I | i-Pr | i-Pr | 4-Cl | 112.0-115.0 |
| 1-003 | 3-I | i-Pr | CF₃ | 4-F | 192.0-194.0 |
| 1-004 | 3-I | i-Pr | CF₃ | 3-Cl | *1 |
| 1-005 | 3-I | i-Pr | CF₃ | 4-Cl | 107.5-109.5 |
| 1-006 | 3-I | i-Pr | CF₃ | 4-Br | 110.0-113.0 |
| 1-007 | 3-I | i-Pr | CF₃ | 4-CF₃ | 112.0-114.0 |
| 1-008 | 3-I | i-Pr | CF₃ | 4-OCH₃ | 102.0-105.0 |
| 1-009 | 3-I | i-Pr | CF₃ | 4-OCF₃ | *1 |
| 1-010 | 3-I | i-Pr | CF₃ | 4-OCF₂Br | 97.0-102.5 |
| 1-011 | 3-I | i-Pr | CF₃ | 4-OCF₂CHF₂ | 98.0-101.0 |
| 1-012 | 3-I | i-Pr | CF₃ | 4-OCF₂CHFCF₃ | 98.0-101.0 |
| 1-013 | 3-I | i-Pr | CF₃ | 4-OCF₂CHFOCF₃ | 93.0-96.0 |
| 1-014 | 3-I | i-Pr | CF₃ | 4-OCF₂CHFOCF₂CF₂CF₃ | 78.0-80.0 |
| 1-015 | 3-I | i-Pr | CF₃ | 4-OSO₂CH₃ | 111.0-114.0 |
| 1-016 | 3-I | i-Pr | CF₃ | 3-O(L-45e) | 103.5-107.0 |
| 1-017 | 3-I | i-Pr | CF₃ | 4-O(L-45e) | 107.0-109.0 |
| 1-018 | 3-I | i-Pr | CF₃ | 4-SCH₃ | 100.0-107.5 |
| 1-019 | 3-I | i-Pr | CF₃ | 4-SO₂CH₃ | 150.0-162.0 |
| 1-020 | 3-I | i-Pr | CF₃ | 2-F-4-Cl | *1 |
| 1-021 | 3-I | i-Pr | CF₃ | 2,4-Cl | 120.0-124.0 |
| 1-022 | 3-I | i-Pr | CF₃ | 3,4-Cl₂ | 112.0-113.0 |
| 1-023 | 3-I | i-Pr | CF₃ | 3,5-Cl₂ | 195.0-197.0 |
| 1-024 | 3-I | i-Pr | CF₃ | 3,4-Br₂ | 125.0-131.5 |
| 1-025 | 3-I | i-Pr | CF₃ | 3-Cl-4-OCHF₂ | *1 |
| 1-026 | 3-I | i-Pr | CF₃ | 3-CH₃-4-OCF₂CHFOCF₃ | 92.0-94.0 |
| 1-027 | 3-I | i-Pr | CF₃ | 3-OCF₂O-4 | *1 |
| 1-028 | 3-I | i-Pr | CF₃ | 3-OCF₂CF₂O-4 | *1 |
| 1-029 | 3-I | i-Pr | CF₃ | 3,5-Cl₂-4-OCH₃ | 110.0-116.5 |
| 1-030 | 3-I | i-Pr | C(O)OCH₃ | 4-Cl | 133.5-135.0 |
| 1-031 | 3-I | s-Bu | CF₃ | 4-OCF₃ | *1 |
| 1-032 | 3-I | t-Bu | CF₃ | 4-OCF₃ | 151.0-154.0 |
| 1-033 | 3-I | CH(CH₃)CH₂SCH₃ | i-Pr | 4-Cl | 105.0-107.0 |
| 1-034 | 3-I | CH(CH₃)CH₂SCH₃ | CF₃ | 4-F | 127.0-130.0 |
| 1-035 | 3-I | CH(CH₃)CH₂SCH₃ | CF₃ | 3-Cl | *1 |
| 1-036 | 3-I | CH(CH₃)CH₂SCH₃ | CF₃ | 4-Cl | 95.5-99.0 |
| 1-037 | 3-I | CH(CH₃)CH₂SCH₃ | CF₃ | 4-Bu-t | 107.0-111.0 |
| 1-038 | 3-I | CH(CH₃)CH₂SCH₃ | CF₃ | 4-CF₃ | 101.0-103.0 |
| 1-039 | 3-I | CH(CH₃)CH₂SCH₃ | CF₃ | 4-OCH₃ | 97.0-99.0 |
| 1-040 | 3-I | CH(CH₃)CH₂SCH₃ | CF₃ | 4-OCF₃ | 112.0-115.0 |
| 1-041 | 3-I | CH(CH₃)CH₂SCH₃ | CF₃ | 4-OCF₂Br | *1 |
| 1-042 | 3-I | CH(CH₃)CH₂SCH₃ | CF₃ | 4-OCF₂CHF₂ | 93.0-96.0 |
| 1-043 | 3-I | CH(CH₃)CH₂SCH₃ | CF₃ | 4-OCF₂CHFCF₃ | 91.0-95.0 |
| 1-044 | 3-I | CH(CH₃)CH₂SCH₃ | CF₃ | 4-OCF₂CHFOCF₃ | 86.0-89.0 |
| 1-045 | 3-I | CH(CH₃)CH₂SCH₃ | CF₃ | 4-OCF₂CHFOCF₂CF₂CF₃ | 71.0-72.0 |
| 1-046 | 3-I | CH(CH₃)CH₂SCH₃ | CF₃ | 4-OSO₂CH₃ | 97.0-100.0 |
| 1-047 | 3-I | CH(CH₃)CH₂SCH₃ | CF₃ | 3-O(L-45e) | *1 |
| 1-048 | 3-I | CH(CH₃)CH₂SCH₃ | CF₃ | 4-O(L-45e) | 105.0-107.0 |
| 1-049 | 3-I | CH(CH₃)CH₂SCH₃ | CF₃ | 4-SO₂CH₃ | 117.0-120.0 |
| 1-050 | 3-I | CH(CH₃)CH₂SCH₃ | CF₃ | 2-F-4-Cl | 116.0-120.0 |
| 1-051 | 3-I | CH(CH₃)CH₂SCH₃ | CF₃ | 2,4-Cl₂ | 112.0-116.0 |
| 1-052 | 3-I | CH(CH₃)CH₂SCH₃ | CF₃ | 3,4-Cl₂ | 99.0-103.0 |
| 1-053 | 3-I | CH(CH₃)CH₂SCH₃ | CF₃ | 3,5-Cl₂ | 121.0-123.5 |
| 1-054 | 3-I | CH(CH₃)CH₂SCH₃ | CF₃ | 3,4-Br₂ | 108.0-110.0 |
| 1-055 | 3-I | CH(CH₃)CH₂SCH₃ | CF₃ | 3-Cl-4-OCHF₂ | *1 |
| 1-056 | 3-I | CH(CH₃)CH₂SCH₃ | CF₃ | 3-Cl-4-OCF₃ | 83.0-89.0 |
| 1-057 | 3-I | CH(CH₃)CH₂SCH₃ | CF₃ | 3-CH₃-4-OCF₂CHFOCF₃ | 86.0-89.0 |
| 1-058 | 3-I | CH(CH₃)CH₂SCH₃ | CF₃ | 3-OCF₂O-4 | *1 |
| 1-059 | 3-I | CH(CH₃)CH₂SCH₃ | CF₃ | 3-OCF₂CF₂O-4 | *1 |
| 1-060 | 3-I | CH(CH₃)CH₂SCH₃ | CF₃ | 3,5-Cl₂-4-OCH₃ | 110.0-113.5 |
| 1-061 | 3-I | CH(CH₃)CH₂SCH₃ | C(O)OCH₃ | 4-Cl | *1 |
| 1-062 | 3-I | CH(CH₃)CH₂S(O)CH₃ | CF₃ | 3-Cl | 182.0-184.0 |
| 1-063 | 3-I | CH(CH₃)CH₂S(O)CH₃ | CF₃ | 4-OCF₃ | 229.0-231.0 |
| 1-064 | 3-I | CH(CH₃)CH₂SO₂CH₃ | i-Pr | 4-Cl | 122.0-124.0 |
| 1-065 | 3-I | CH(CH₃)CH₂SO₂CH₃ | CF₃ | 4-F | 142.0-144.0 |
| 1-066 | 3-I | CH(CH₃)CH₂SO₂CH₃ | CF₃ | 4-Cl | 180.0-184.0 |
| 1-067 | 3-I | CH(CH₃)CH₂SO₂CH₃ | CF₃ | 4-CF₃ | 111.0-113.0 |
| 1-068 | 3-I | CH(CH₃)CH₂SO₂CH₃ | CF₃ | 4-OCH₃ | 123.0-126.0 |
| 1-069 | 3-I | CH(CH₃)CH₂SO₂CH₃ | CF₃ | 4-OCF₃ | 218.0-220.0 |
| 1-070 | 3-I | CH(CH₃)CH₂SO₂CH₃ | CF₃ | 4-OCF₂Br | 115.0-117.5 |
| 1-071 | 3-I | CH(CH₃)CH₂SO₂CH₃ | CF₃ | 4-OCF₂CHF₂ | 112.0-115.0 |
| 1-072 | 3-I | CH(CH₃)CH₂SO₂CH₃ | CF₃ | 4-OCF₂CHFCF₃ | 144.0-146.0 |
| 1-073 | 3-I | CH(CH₃)CH₂SO₂CH₃ | CF₃ | 4-OCF₂CHFOCF₃ | 102.0-105.0 |
| 1-074 | 3-I | CH(CH₃)CH₂SO₂CH₃ | CF₃ | 4-O(L-45e) | 123.0-124.0 |
| 1-075 | 3-I | CH(CH₃)CH₂SO₂CH₃ | CF₃ | 2-F-4-Cl | 228.0-230.0 |
| 1-076 | 3-I | CH(CH₃)CH₂SO₂CH₃ | CF₃ | 2,4-Cl₂ | 125.0-128.0 |
| 1-077 | 3-I | CH(CH₃)CH₂SO₂CH₃ | CF₃ | 3,4-Cl₂ | 112.0-114.0 |
| 1-078 | 3-I | CH(CH₃)CH₂SO₂CH₃ | CF₃ | 3,5-Cl₂ | 127.0-132.0 |
| 1-079 | 3-I | CH(CH₃)CH₂SO₂CH₃ | CF₃ | 3,4-Br₂ | 133.0-145.0 |
| 1-080 | 3-I | CH(CH₃)CH₂SO₂CH₃ | CF₃ | 3-Cl-4-OCHF₂ | *1 |
| 1-081 | 3-I | CH(CH₃)CH₂SO₂CH₃ | CF₃ | 3-CH₃-4-OCF₂CHFOCF₃ | 116.0-118.0 |
| 1-082 | 3-I | CH(CH₃)CH₂SO₂CH₃ | CF₃ | 3-OCF₂O-4 | 193.0-203.0 |
| 1-083 | 3-I | CH(CH₃)CH₂SO₂CH₃ | CF₃ | 3-OCF₂CF₂O-4 | *1 |
| 1-084 | 3-I | C(CH₃)₂CH₂SCH₃ | CF₃ | 4-Cl | 172.0-175.0 |
| 1-085 | 3-I | C(CH₃)₂CH₂SCH₃ | CF₃ | 4-OCF₃ | *1 |
| 1-086 | 3-I | C(CH₃)₂CH₂SCH₃ | CF₃ | 3,4-Cl₂ | 96.0-98.0 |
| 1-087 | 3-I | C(CH₃)₂CH₂SO₂CH₃ | CF₃ | 4-Cl | 126.0-128.0 |
| 1-088 | 3-I | C(CH₃)₂CH₂SO₂CH₃ | CF₃ | 4-OCF₃ | *1 |
| 1-089 | 3-I | C(CH₃)₂CH₂SO₂CH₃ | CF₃ | 3,4-Cl₂ | 109.0-113.0 |
| 1-090 | 3-I | C(CH₃)₂C≡CH | CF₃ | 4-OCF₃ | *1 |

TABLE 7

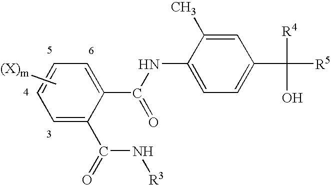

| No. | (X)ₘ | R³ | R⁴ | R⁵ | m.p. (° C.) |
|---|---|---|---|---|---|
| 2-001 | 3-I | i-Pr | CH₃ | (L-45c)CF₃ | *1 |
| 2-002 | 3-I | i-Pr | CF₃ | CH₂CH₂(Ph-4-Cl) | 103.5-106.0 |
| 2-003 | 3-I | i-Pr | CF₃ | CH₂OCH₃ | 181.0-184.0 |
| 2-004 | 3-I | i-Pr | CF₃ | CH₂O(Ph-4-Cl) | 90.0-95.0 |
| 2-005 | 3-I | i-Pr | CF₃ | CH₂OC(O)(Ph-4-Cl) | 180.0-183.0 |
| 2-006 | 3-I | i-Pr | CF₃ | CF₂O(Ph-2-Cl) | 86.0-88.0 |
| 2-007 | 3-I | i-Pr | CF₃ | CF₂O(Ph-3-Cl) | 82.0-85.0 |
| 2-008 | 3-I | i-Pr | CF₃ | CF₂O(Ph-4-Cl) | 65.0-75.0 |
| 2-009 | 3-I | i-Pr | CF₃ | CH₂S(Ph-4-Cl) | 80.0-83.0 |
| 2-010 | 3-I | i-Pr | CF₃ | CH₂SO₂(Ph-4-Cl) | 111.0-113.0 |
| 2-011 | 3-I | i-Pr | CF₃ | CH₂N(CH₃)₂ | 204.0-207.0 |
| 2-012 | 3-I | i-Pr | CF₃ | CH₂NH(Ph-4-Cl) | 120.0-122.0 |
| 2-013 | 3-I | i-Pr | CF₃ | CF₂C(O)OEt | *1 |
| 2-014 | 3-I | i-Pr | CF₃ | CH₂(L-36a) | 149.0-152.0 |
| 2-015 | 3-I | i-Pr | CF₃ | C(O)OEt | 177.0-178.0 |
| 2-016 | 3-I | i-Pr | CF₃ | CH═CH(Ph-4-F) | 109.0-111.0 |
| 2-017 | 3-I | i-Pr | CF₃ | CH═CH(Ph-3-Cl) | *1 |
| 2-018 | 3-I | i-Pr | CF₃ | CH═CH(Ph-4-Cl) | 108.0-113.0 |
| 2-019 | 3-I | i-Pr | CF₃ | CH═CH(Ph-4-OCF₃) | 104.0-108.5 |
| 2-020 | 3-I | i-Pr | CF₃ | CH═CH(Ph-4-SCH₃) | *1 |
| 2-021 | 3-I | i-Pr | CF₃ | CH═CH(Ph-4-SO₂CH₃) | 133.0-138.0 |
| 2-022 | 3-I | i-Pr | CF₃ | CH═CH(Ph-3,4-Cl₂) | 111.0-113.0 |
| 2-023 | 3-I | i-Pr | CF₃ | 2-Naph | 113.0-117.0 |
| 2-024 | 3-I | i-Pr | CF₃ | (L-45c)CF₃ | 107.0-110.0 |
| 2-025 | 3-I | CH(CH₃)CH₂SCH₃ | CF₃ | CF₂O(Ph-4-Cl) | *1 |
| 2-026 | 3-I | CH(CH₃)CH₂SCH₃ | CF₃ | CH═CH(Ph-4-F) | 98.0-111.0 |
| 2-027 | 3-I | CH(CH₃)CH₂SCH₃ | CF₃ | CH═CH(Ph-3-Cl) | *1 |
| 2-028 | 3-I | CH(CH₃)CH₂SCH₃ | CF₃ | CH═CH(Ph-4-Cl) | 103.5-108.0 |
| 2-029 | 3-I | CH(CH₃)CH₂SCH₃ | CF₃ | CH═CH(Ph-4-OCF₃) | *1 |
| 2-030 | 3-I | CH(CH₃)CH₂SCH₃ | CF₃ | CH═CH(Ph-3,4-Cl₂) | 98.0-101.0 |
| 2-031 | 3-I | CH(CH₃)CH₂SCH₃ | CF₃ | 2-Naph | *1 |
| 2-032 | 3-I | CH(CH₃)CH₂SCH₃ | CF₃ | (L-15b)CF₃ | 94.0-97.0 |
| 2-033 | 3-I | CH(CH₃)CH₂SO₂CH₃ | CF₃ | CF₂O(Ph-4-Cl) | 131.5-135.0 |
| 2-034 | 3-I | CH(CH₃)CH₂SO₂CH₃ | CF₃ | CH═CH(Ph-4-F) | 123.0-125.0 |
| 2-035 | 3-I | CH(CH₃)CH₂SO₂CH₃ | CF₃ | CH═CH(Ph-4-Cl) | 128.0-130.5 |
| 2-036 | 3-I | CH(CH₃)CH₂SO₂CH₃ | CF₃ | CH═CH(Ph-4-OCF₃) | 120.5-125.0 |
| 2-037 | 3-I | CH(CH₃)CH₂SO₂CH₃ | CF₃ | CH═CH(Ph-3,4-Cl₂) | 124.0-128.0 |
| 2-038 | 3-I | CH(CH₃)CH₂SO₂CH₃ | CF₃ | 2-Naph | *1 |

TABLE 8

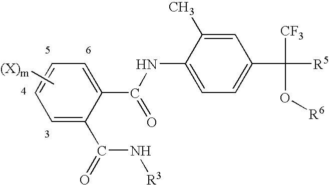

| No. | (X)ₘ | R³ | R⁵ | R⁶ | m.p. (° C.) |
|---|---|---|---|---|---|
| 3-001 | 3-I | i-Pr | CF₂O(Ph-4-Cl) | CH₃ | 55.0-65.0 |
| 3-002 | 3-I | i-Pr | Ph-4-Cl | CH₃ | 211.5-214.0 |
| 3-003 | 3-I | i-Pr | Ph-4-Cl | CH₂OCH₃ | 226.5-229.0 |
| 3-004 | 3-I | i-Pr | Ph-4-Cl | C(O)CH₃ | 212.0-214.0 |
| 3-005 | 3-I | i-Pr | CH═CH(Ph-4-Cl) | CH₃ | *1 |
| 3-006 | 3-I | CH(CH₃)CH₂SCH₃ | CH═CH(Ph-4-Cl) | CH₃ | *1 |
| 3-007 | 3-I | CH(CH₃)CH₂SCH₃ | CH═CH(Ph-4-Cl) | CH₂OCH₃ | *1 |
| 3-008 | 3-I | CH(CH₃)CH₂SCH₃ | Ph-4-Cl | CH₃ | 103.0-107.0 |
| 3-009 | 3-I | CH(CH₃)CH₂SCH₃ | Ph-4-Cl | CH₂OCH₃ | 185.0-188.5 |
| 3-010 | 3-I | CH(CH₃)CH₂SCH₃ | Ph-4-Cl | Si(CH₃)₃ | 113.0-116.0 |
| 3-011 | 3-I | CH(CH₃)CH₂SCH₃ | Ph-4-OCH₃ | Si(CH₃)₃ | 99.0-103.0 |
| 3-012 | 3-I | CH(CH₃)CH₂SO₂CH₃ | CH═CH(Ph-4-Cl) | CH₃ | *1 |
| 3-013 | 3-I | CH(CH₃)CH₂SO₂CH₃ | CH═CH(Ph-4-Cl) | CH₂OCH₃ | *1 |
| 3-014 | 3-I | CH(CH₃)CH₂SO₂CH₃ | Ph-4-Cl | CH₃ | 111.0-114.0 |
| 3-015 | 3-I | CH(CH₃)CH₂SO₂CH₃ | Ph-4-Cl | CH₂OCH₃ | *1 |
| 3-016 | 3-I | CH(CH₃)CH₂SO₂CH₃ | Ph-4-OCH₃ | Si(CH₃)₃ | 115.0-119.0 |

TABLE 9

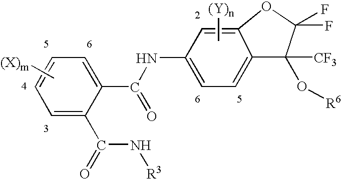

| No. | (X)ₘ | R³ | (Y)ₙ | R⁶ | m.p. (° C.) |
|---|---|---|---|---|---|
| 4-001 | 3-I | i-Pr | 2-CH₃ | H | 123.0-126.0 |
| 4-002 | 3-I | i-Pr | 6-CH₃ | H | 144.0-146.0 |
| 4-003 | 3-I | i-Pr | 6-CH₃ | Ph-4-Cl | 195.0-198.0 |

TABLE 10

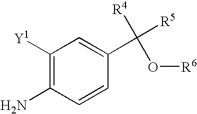

| No. | Y¹ | R⁴ | R⁵ | R⁶ | m.p. (° C.) |
|---|---|---|---|---|---|
| 5-01 | CH₃ | CH₃ | (L-45c)CF₃ | H | *2 |
| 5-02 | CH₃ | CF₃ | C(O)OEt | H | *2 |
| 5-03 | CH₃ | CF₃ | CH═CH(Ph-4-F) | H | 97.0-99.0 |
| 5-04 | CH₃ | CF₃ | Ph-4-Cl | H | 157.0-160.5 |
| 5-05 | CH₃ | CF₃ | Ph-4-Cl | CH₃ | *2 |
| 5-06 | CH₃ | CF₃ | Ph-4-OCH₃ | Si(CH₃)₃ | *2 |
| 5-07 | CH₃ | CF₃ | Ph-4-OCF₂CHF₂ | H | 69.0-72.0 |
| 5-08 | CH₃ | CF₃ | Ph-4-OCF₂CHFOCF₃ | H | 70.0-75.0 |
| 5-09 | CH₃ | CF₃ | Ph-4-OCF₂CHFOCF₂CF₂CF₃ | H | *2 |
| 5-10 | CH₃ | CF₃ | Ph-4-OSO₂CH₃ | H | 137.0-139.0 |
| 5-11 | CH₃ | CF₃ | Ph-2,4-Cl₂ | H | 149.0-151.0 |
| 5-12 | CH₃ | CF₃ | Ph-3-CH₃-4-OCF₂CHFOCF₃ | H | 84.0-87.0 |
| 5-13 | CH₃ | CF₃ | Ph-3-OCF₂CF₂O-4 | H | 147.5-150.0 |
| 5-14 | CH₃ | CF₃ | (L-45c)CF₃ | H | 136.0-138.5 |

### TEST EXAMPLES

Next, usefulness of the compound of the present invention as a noxious organism controlling agent is specifically explained in the following Test Examples, but the present invention is not limited by these alone.

### Test Example 1

### Insecticidal Test Against Diamondback Moth

A 10% emulsifiable concentrate (depending on the compounds, 25% wettable powder was applied for the test) of the compound of the present invention was diluted with water containing a spreading agent to prepare a chemical solution with a concentration of 100 ppm. To the chemical solution was dipped leaves of Chinese olive for about 10 seconds, and after air-drying, they were placed in a laboratory dish, then, 10 diamondback moths (Plutella xylostella) with second instar larvae per the dish were released therein, and the dish was covered with a lid having holes and contained at a thermostat chamber at 25° C. A number of dead insect(s) after 6 days was counted and a rate of dead insects was calculated in the same manner as in Test Example 1. Incidentally, the test was carried out with two districts.

As a result, the following compounds showed 100% of insecticidal rate.

The compounds of the present invention: No. 1-001, 1-002, 1-003, 1-004, 1-005, 1-006, 1-007, 1-008, 1-009, 1-010, 1-011, 1-012, 1-013, 1-014, 1-015, 1-016, 1-017, 1-018, 1-019, 1-020, 1-021, 1-022, 1-023, 1-024, 1-025, 1-026, 1-027, 1-028, 1-029, 1-030, 1-031, 1-032, 1-033, 1-034, 1-035, 1-036, 1-037, 1-038, 1-039, 1-040, 1-041, 1-042, 1-043, 1-044, 1-045, 1-046, 1-047, 1-048, 1-050, 1-051, 1-052, 1-053, 1-054, 1-055, 1-056, 1-057, 1-058, 1-059, 1-060, 1-061, 1-062, 1-063, 1-064, 1-065, 1-066, 1-067, 1-068, 1-069, 1-070, 1-071, 1-072, 1-073, 1-074, 1-075, 1-076, 1-077, 1-078, 1-079, 1-080, 1-081, 1-082, 1-083, 1-084, 1-085, 1-086, 1-087, 1-088, 1-089, 1-090, 2-001, 2-002, 2-003, 2-004, 2-005, 2-006, 2-007, 2-008, 2-009, 2-010, 2-011, 2-012, 2-013, 2-014, 2-015, 2-016, 2-017, 2-018, 2-019, 2-020, 2-021, 2-022, 2-023, 2-024, 2-025, 2-026, 2-027, 2-028, 2-029, 2-030, 2-031, 2-032, 2-033, 2-034, 2-035, 2-036, 2-037, 2-038, 3-001, 3-002, 3-004, 3-005, 3-006, 3-007, 3-008, 3-009, 3-010, 3-011, 3-012, 3-013, 3-014, 3-015, 4-001, 4-002.

### Test Example 2

### Insecticidal Test Against Common Cutworm

A 10% emulsifiable concentrate (depending on the compounds, 25% wettable powder was applied for the test) of the compound of the present invention was diluted with water containing a spreading agent to prepare a chemical solution with a concentration of 100 ppm. To the chemical solution was dipped leaves of Chinese olive for about 10 seconds, and after air-drying, they were placed in a laboratory dish, then, 10 Common cutworms (Spodoptera litura) with second instar larvae per the dish were released therein, and the dish was covered with a lid having holes and contained at a thermostat chamber at 25° C. A number of dead insect(s) after 6 days was counted and a rate of dead insects was calculated by the following calculation formula. Incidentally, the test was carried out with two districts.
Rate of dead insects (%)=(Number of dead insects/Number of released insects)×100

As a result, the following compounds showed 80% or more of insecticidal rate.

The compounds of the present invention: No. 1-001, 1-003, 1-005, 1-006, 1-007, 1-009, 1-010, 1-011, 1-012, 1-013, 1-014, 1-015, 1-016, 1-017, 1-020, 1-021, 1-023, 1-024, 1-025, 1-026, 1-027, 1-028, 1-029, 1-031, 1-032, 1-034, 1-035, 1-036, 1-038, 1-040, 1-041, 1-042, 1-043, 1-044, 1-045, 1-047, 1-048, 1-050, 1-051, 1-052, 1-053, 1-054, 1-055, 1-056, 1-057, 1-058, 1-059, 1-063, 1-065, 1-066, 1-067, 1-069, 1-070, 1-071, 1-072, 1-073, 1-074, 1-075, 1-076, 1-077, 1-078, 1-079, 1-080, 1-081, 1-082, 1-083, 1-084, 1-085, 1-086, 1-087, 1-088, 1-089, 1-090, 2-001, 2-004, 2-007, 2-008, 2-012, 2-016, 2-017, 2-018, 2-019, 2-020, 2-022, 2-024, 2-025, 2-026, 2-027, 2-028, 2-029, 2-030, 2-033, 2-034, 2-035, 2-036, 2-037, 3-001, 3-002, 3-004, 3-005, 3-006, 3-007, 3-008, 3-009, 3-010, 3-012, 3-013, 3-014, 3-015, 4-001, 4-002.

### Test Example 3

### Insecticidal Test Against Oriental Tea Tortrix

A 10% emulsifiable concentrate (depending on the compounds, 10% wettable powder was applied for the test) of the compound of the present invention was diluted with water containing a spreading agent to prepare a chemical solution with a concentration of 100 ppm. To the chemical solution was dipped leaves of Chinese olive for about 10 seconds, and after air-drying, they were placed in a laboratory dish, then, 5 Oriental tea tortrix (Homona magnanima) with second instar larvae per the dish were released therein, and the dish was covered with a lid having holes and contained at a thermostat chamber at 25° C. A number of dead insect(s) after 6 days was counted and a rate of dead insects was calculated in the same manner as in Test Example 1. Incidentally, the test was carried out with two districts.

As a result, the following compounds showed 80% or more of insecticidal rate.

The compounds of the present invention: No. 1-001, 1-003, 1-004, 1-005, 1-006, 1-007, 1-009, 1-010, 1-011, 1-012, 1-013, 1-017, 1-020, 1-021, 1-022, 1-023, 1-024, 1-025, 1-026, 1-027, 1-028, 1-032, 1-034, 1-035, 1-036, 1-038, 1-040, 1-041, 1-042, 1-043, 1-044, 1-045, 1-048, 1-050, 1-051, 1-052, 1-053, 1-054, 1-056, 1-057, 1-058, 1-059, 1-062, 1-063, 1-065, 1-066, 1-067, 1-069, 1-070, 1-071, 1-072, 1-073, 1-075, 1-076, 1-077, 1-078, 1-079, 1-081, 1-082, 1-083, 1-084, 1-085, 1-086, 1-088, 1-089, 2-008, 2-016, 2-018, 2-019, 2-024, 2-029, 2-033, 2-035, 3-001, 3-002, 3-008, 3-009, 3-014, 3-015, 4-001, 4-002.

### Test Example 4

### Insecticidal Test Against Cucurbit Leaf Beetle

A 10% emulsifiable concentrate (depending on the compounds, 10% wettable powder was applied for the test) of the compound of the present invention was diluted with water containing a spreading agent to prepare a chemical solution with a concentration of 500 ppm. To the chemical solution was dipped leaves of cucumber for about 10 seconds, and after air-drying, they were placed in a laboratory dish, then, 5 Cucurbit leaf beetle (Aulacophora femoralis) with second instar larvae per the dish were released therein, and the dish was covered with a lid having holes and contained at a thermostat chamber at 25° C. A number of dead insect(s) after 6 days was counted and a rate of dead insects was calculated in the same manner as in Test Example 1. Incidentally, the test was carried out with two districts.

As a result, the following compounds showed 100% of insecticidal rate.

The compounds of the present invention: No. 1-001, 1-004, 1-005, 1-006, 1-007, 1-009, 1-010, 1-011, 1-012, 1-013, 1-014, 1-016, 1-017, 1-018, 1-020, 1-022, 1-023, 1-024, 1-025, 1-026, 1-027, 1-028, 1-031, 1-032, 1-035, 1-036, 1-038, 1-040, 1-041, 1-042, 1-043, 1-044, 1-045, 1-047, 1-048, 1-050, 1-052, 1-053, 1-054, 1-055, 1-056, 1-057, 1-058, 1-059, 1-062, 1-063, 1-064, 1-066, 1-067, 1-068, 1-069, 1-070, 1-071, 1-072, 1-073, 1-074, 1-075, 1-077, 1-078, 1-079, 1-080, 1-081, 1-082, 1-083, 1-084, 1-085, 1-086, 1-087, 1-088, 1-089, 2-001, 2-004, 2-008, 2-009, 2-010, 2-016, 2-017, 2-018, 2-019, 2-021, 2-022, 2-024, 2-025, 2-026, 2-028, 2-029, 2-030, 2-033, 2-034, 2-035, 2-036, 2-037, 3-001, 3-005, 3-006, 3-007, 3-008, 3-010, 3-012, 3-013, 3-014, 4-001, 4-002.

### Test Example 5

### Insecticidal Test (Comparative Test) Against Common Cutworm

10% emulsifiable concentrates of the compound of the present invention and comparative compound were diluted with water containing a spreading agent to prepare chemical solutions with a predetermined concentration. To the chemical solutions was dipped leaves of Chinese olive for about 10 seconds, and after air-drying, they were placed in a laboratory dish, then, 7 Common cutworm (Spodoptera litura) with third instar larvae per the dish were released therein, and the dish was covered with a lid having holes and contained at a thermostat chamber at 25° C. Artificial feed (1 cm³) was added after 2 days, a number of dead insect(s) was counted after 6 days and a rate of dead insects was calculated by the following calculation formula. Incidentally, the test was carried out with two districts.
Rate of dead insects (%)=(Number of dead insects/Number of released insects)×100

Rate of dead insects at the respective concentrations of the respective test compounds are shown in Table 11.

TABLE 11

| | Concentration (ppm) | | | | | |
|---|---|---|---|---|---|---|
| Test compounds | 33 | 10 | 3 | 1 | 0.3 | 0.1 |
| Present compound No. 1-036 | 100 | 100 | 100 | 85.7 | 28.6 | 14.3 |
| Present compound No. 1-084 | 100 | 100 | 71.4 | 21.3 | 0 | |
| Present compound No. 1-087 | 100 | 100 | 100 | 71.4 | 21.3 | 0 |
| Comparative compound A | 0 | | | | | |

Comparative compound A: EP 1006107 A, Compound No. 472

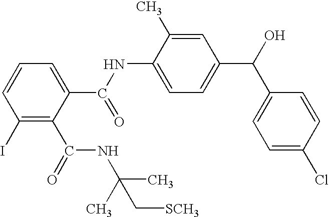

### UTILIZABLE FIELD IN INDUSTRY

The substituted benzanilide compound according to the present invention is an extremely useful compound which shows excellent noxious organism controlling activity, in particular insecticidal and acaricidal activities, and causes substantially no bad effect on non-target creatures such as mammals, fishes and useful insects, etc.

1. A substituted benzanilide compound represented by the formula (1): 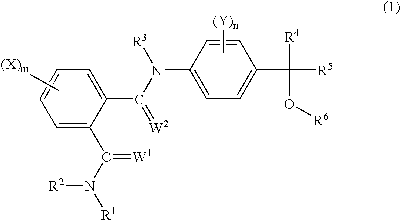
wherein W¹ and W² each independently represent an oxygen atom or a sulfur atom,
X represents a halogen atom, cyano, nitro, azide, —SCN, —SF₅, a C₁ to C₆ alkyl, a (C₁ to C₆) alkyl optionally substituted by R⁷, a C₃ to C₈ cycloalkyl, a (C₃ to C₈) cycloalkyl optionally substituted by R⁷, a C₂ to C₆ alkenyl, a (C₂ to C₆) alkenyl optionally substituted by R⁷, a C₃ to C₈ cycloalkenyl, a C₃ to C₈ halocycloalkenyl, a C₂ to C₆ alkynyl, a (C₂ to C₆) alkynyl optionally substituted by R⁷, —OH, —OR⁸, —OS(O)₂R⁸, —SH, S(O)ᵣR⁸, —CHO, —C(O)R⁹, —C(O)OR⁹, —C(O)SR⁹, —C(O)NHR¹⁰, —C(O)N(R¹⁰)R⁹, —C(S)OR⁹, —C(S)SR⁹, —C(S)NHR¹⁰,
—C(S)N(R¹⁰)R⁹, —CH═NOR¹¹, —C(R⁹)═NOR¹¹, —S(O)₂OR⁹, —S(O)₂NHR¹⁰, —S(O)₂N(R¹⁰)R⁹,
—Si(R¹³)(R¹⁴)R¹², phenyl, a phenyl substituted by (Z)ₚ₁, L or M, when m is 2, 3 or 4, each X may be the same or different from each other, and when two Xs are adjacent to each other, the adjacent two Xs may form a 5-membered ring or 6-membered ring with the carbon atoms to which two Xs are bonded by forming —CH₂CH₂CH₂—, —CH₂CH₂O—,
—CH₂OCH₂—, —OCH₂O—, —CH₂CH₂S—, —CH₂SCH₂—, —CH₂CH₂N(R¹⁵)—, —CH₂N(R¹⁵)CH₂—, —CH₂CH₂CH₂CH₂—, —CH₂CH₂CH₂O—, —CH₂CH₂OCH₂—, —CH₂OCH₂O—, —OCH₂CH₂O—, —OCH₂CH₂S—, —CH₂CH═CH—, —OCH═CH—, —SCH═CH—, —N(R¹⁵)CH═CH—, —OCH═N—, —SCH═N—, —N(R¹⁵)CH═N—, —N(R¹⁵)N═CH—, —CH═CHCH═CH—, —OCH₂CH═CH—, —N═CHCH═CH—, —N═CHCH═N— or —N═CHN═CH—, and at this time, each hydrogen atom bonded to the respective carbon atoms which form the ring may be optionally substituted by Z, and further when it is substituted by two or more Zs at the same time, each Z may be the same or different from each other,
Y represents a halogen atom, cyano, nitro, a C₁ to C₆ alkyl, a (C₁ to C₆) alkyl optionally substituted by R⁷, a C₃ to C₈ cycloalkyl, —OR⁸, S(O)ᵣR⁸, —NH₂, a C₁ to C₆ alkylamino, a di(C₁ to C₆ alkyl)amino or —Si(R¹³)(R¹⁴)R¹², when n is 2, 3 or 4, each Y may be the same or different from each other, and when two Ys are adjacent to each other, the adjacent two Ys may form a 5-membered ring or 6-membered ring with the carbon atoms to which two Ys are bonded by forming —CH₂CH₂CH₂—, —CH₂CH₂O—, —CH₂OCH₂—, —OCH₂O—, —CH₂CH₂S—, —CH₂SCH₂—, —SCH₂S—, —CH₂CH₂C H₂CH₂—, —CH₂CH₂CH₂O—, —CH₂CH₂OCH₂—,
—CH₂OCH₂O—, —OCH₂CH₂O—, —OCH₂CH₂S—, —SCH₂CH₂S—, —OCH═N— or —SCH═N—, and at this time, each hydrogen atom bonded to the respective carbon atoms which form the ring may be optionally substituted by Z, and further when it is substituted by two or more Zs at the same time, each Z may be the same or different from each other,
R¹ represents a hydrogen atom, cyano, a C₁ to C₁₂ alkyl, a (C₁ to C₁₂) alkyl optionally substituted by R¹⁶, a C₃ to C₁₂ cycloalkyl, a (C₃ to C₁₂) cycloalkyl optionally substituted by R¹⁶, a C₃ to C₁₂ alkenyl, a (C₃ to C₁₂) alkenyl optionally substituted by R¹⁶, a C₃ to C₁₂ cycloalkenyl, a C₃ to C₁₂ halocycloalkenyl, a C₃ to C₁₂ alkynyl, a (C₃ to C₁₂) alkynyl optionally substituted by R¹⁶, —OH, a C₁ to C₈ alkoxy, a C₃ to C₈ alkenyloxy, a C₃ to C₈ haloalkenyloxy, phenoxy, a phenoxy substituted by (Z)ₚ₁, a phenyl(C₁ to C₄) alkoxy, a phenyl(C₁ to C₄) alkoxy substituted by (Z)ₚ₁, —N(R²⁰)R¹⁹, phenyl, a phenyl substituted by (Z)ₚ₁, L or M,
R² and R³ each independently represent a hydrogen atom, cyano, a C₁ to C₁₂ alkyl, a (C₁ to C₁₂) alkyl optionally substituted by R¹⁶, a C₃ to C₁₂ alkenyl, a C₃ to C₁₂ haloalkenyl, a C₃ to C₁₂ alkynyl, a C₃ to C₁₂ haloalkynyl, —OH, a C₁ to C₈ alkoxy, a C₁ to C₆ alkylthio, a C₁ to C₆ haloalkylthio, phenylthio, a phenylthio substituted by (Z)ₚ₁, —S(O)₂R⁹,
—SN(R¹⁸)R¹⁷, —S(O)₂N(R¹⁰)R⁹, —N(R²⁰)R¹⁹, —C(O)R⁹, —C(O)OR⁹, —C(O)SR⁹, —C(O)N(R¹⁰)R⁹,
—C(S)OR⁹, —C(S)SR⁹, —C(S)N(R¹⁰)R⁹, phenyl or a phenyl substituted by (Z)ₚ₁, or R² is combined with R¹ to form a C₂ to C₆ alkylene chain whereby it may form a 3 to 7-membered ring with the nitrogen atom to which they are bonded, and the alkylene chain at this time may contain one oxygen atom, sulfur atom or nitrogen atom, and may be optionally substituted by a halogen atom, a C₁ to C₆ alkyl group, a C₁ to C₆ haloalkyl group, a C₁ to C₆ alkoxy group, a C₁ to C₆ alkylcarbonyl group or a C₁ to C₆ alkoxycarbonyl group,
R⁴ represents a C₁ to C₆ alkyl, a C₁ to C₆ haloalkyl, a (C₁ to C₆) alkyl optionally substituted by R²¹, a (C₁ to C₆) haloalkyl optionally substituted by R²¹, a C₃ to C₈ cycloalkyl, a C₃ to C₈ halocycloalkyl, a (C₃ to C₈) cycloalkyl optionally substituted by R²¹, a (C₃ to C₈) halocycloalkyl optionally substituted by R²¹, a C₃ to C₆ alkenyl, a C₃ to C₆ haloalkenyl, a C₃ to C₆ alkynyl, a C₃ to C₆ haloalkynyl, phenyl, a phenyl substituted by (Z)ₚ₁, L or M,
R⁵ represents cyano, a (C₁ to C₆) alkyl optionally substituted by R²¹, a (C₁ to C₆) haloalkyl optionally substituted by R²¹, a (C₃ to C₈) cycloalkyl optionally substituted by R²¹, a (C₃ to C₈) halocycloalkyl optionally substituted by R²¹, a (C₂ to C₆) alkenyl optionally substituted by R²¹, a C₃ to C₈ cycloalkenyl, a C₃ to C₈ halocycloalkenyl, a (C₂ to C₆) alkynyl optionally substituted by R²¹, —OR⁸, —S(O)ᵣR⁸, —N(R¹⁰)R⁹, —CHO, —C(O)R⁹, —CH═NOR¹¹,
—C(R⁹)═NOR¹¹, —C(O)OR⁹, —C(O)SR⁹, —C(O)NHR¹⁰, —C(O)N(R¹⁰)R⁹, —C(S)OR⁹, —C(S)SR⁹,
—C(S)NHR¹⁰, —C(S)N(R¹⁰)R⁹, phenyl, a phenyl substituted by (Z)ₚ₁, biphenyl, a biphenyl substituted by (Z)ₚ₁, phenoxyphenyl, a phenoxyphenyl substituted by (Z)ₚ₁, pyridyloxyphenyl, a pyridyloxyphenyl substituted by (Z)ₚ₁, phenylthiophenyl, a phenylthiophenyl substituted by (Z)ₚ₁, phenylsulfinylphenyl, a phenylsulfinylphenyl substituted by (Z)ₚ₁, phenylsulfonylphenyl, a phenylsulfonylphenyl substituted by (Z)ₚ₁, L or M, or it forms a C₂ to C₃ alkylene chain with Y present at the adjacent position in combination whereby it may form a 5 to 6-membered ring which fuses with a benzene ring, and the alkylene chain at this time may contain one oxygen atom, sulfur atom or nitrogen atom, and may be optionally substituted by a halogen atom or a C₁ to C₆ haloalkyl group,
R⁶ represents a hydrogen atom, a C₁ to C₆ alkyl, a C₁ to C₆ haloalkyl, a (C₁ to C₆) alkyl optionally substituted by R²¹, a (C₁ to C₆) haloalkyl optionally substituted by R²¹, a C₃ to C₈ cycloalkyl, a C₃ to C₆ alkenyl, a C₃ to C₆ haloalkenyl, a phenyl(C₃ to C₆) alkenyl, a phenyl(C₃ to C₆) alkenyl substituted by (Z)ₚ₁, a C₃ to C₈ cycloalkenyl, a C₃ to C₆ alkynyl, a C₃ to C₆ haloalkynyl, a phenyl(C₃ to C₆) alkynyl, a phenyl(C₃ to C₆) alkynyl substituted by (Z)ₚ₁, —S(O)₂R⁹, —C(O)R⁹, —C(O)OR⁹, —C(O)SR⁹, —C(S)OR⁹, —C(S)SR⁹, —C(O)NHR¹⁰,
—C(O)N(R¹⁰)R⁹, —C(S)NHR¹⁰, —C(S)N(R¹⁰)R⁹, —Si(R¹³) (R¹⁴)R¹², —P(O)(OR²²)₂, P(S)(OR²²)₂ or M,
L represents an aromatic heterocyclic ring represented by any of the formula L-1 to the formula L-58, 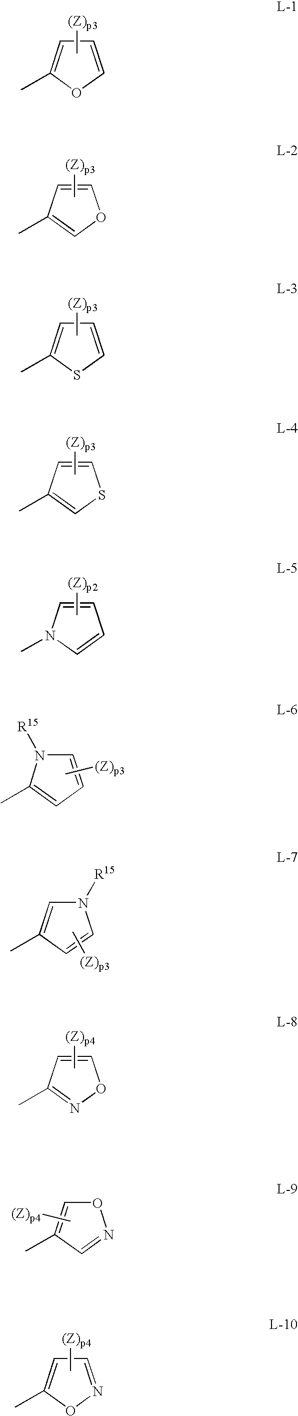 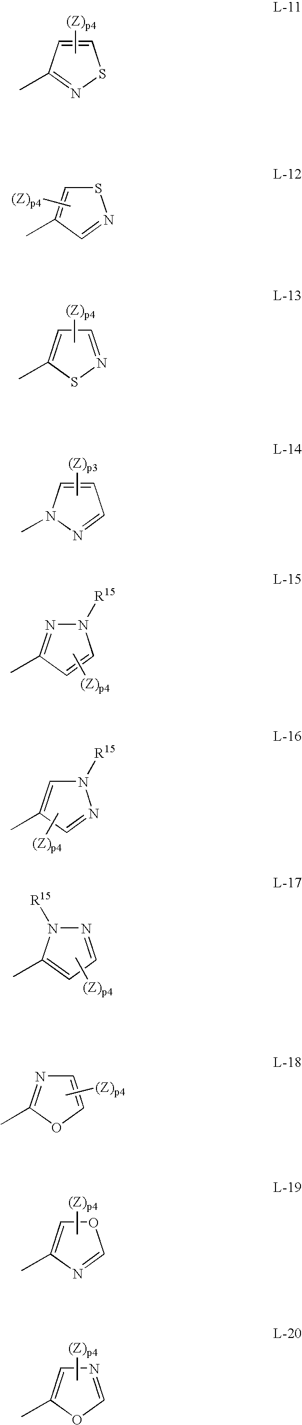 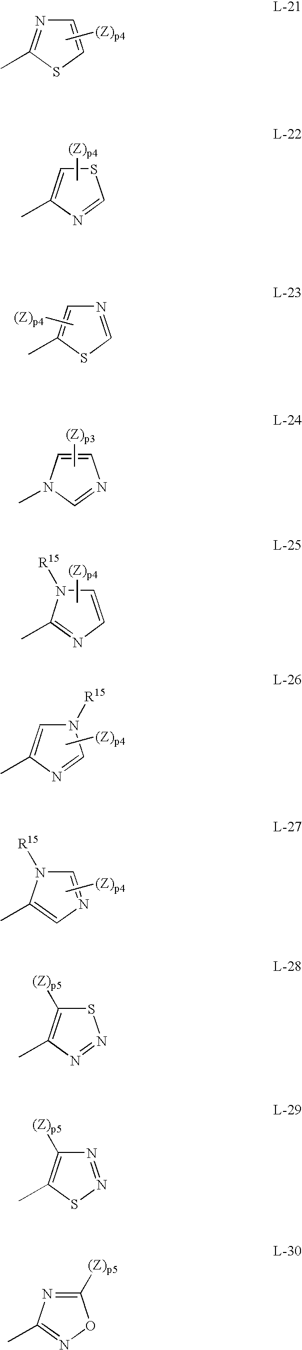 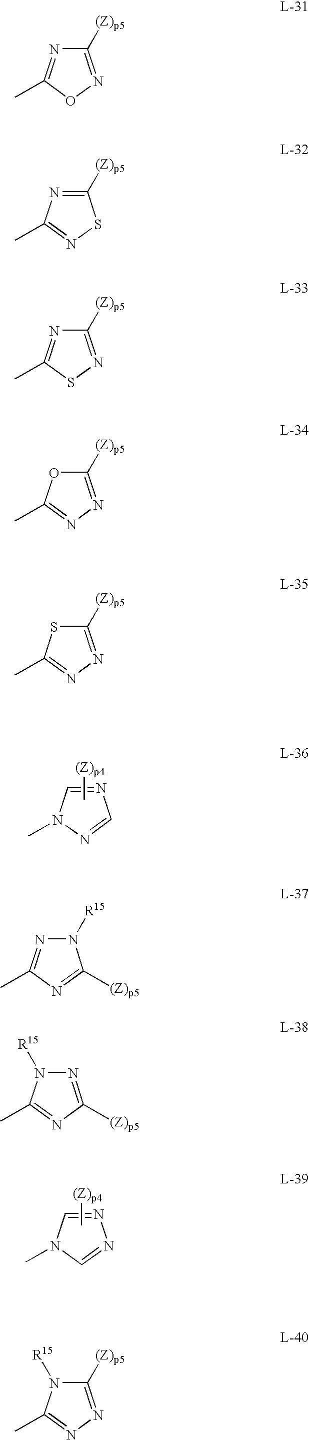 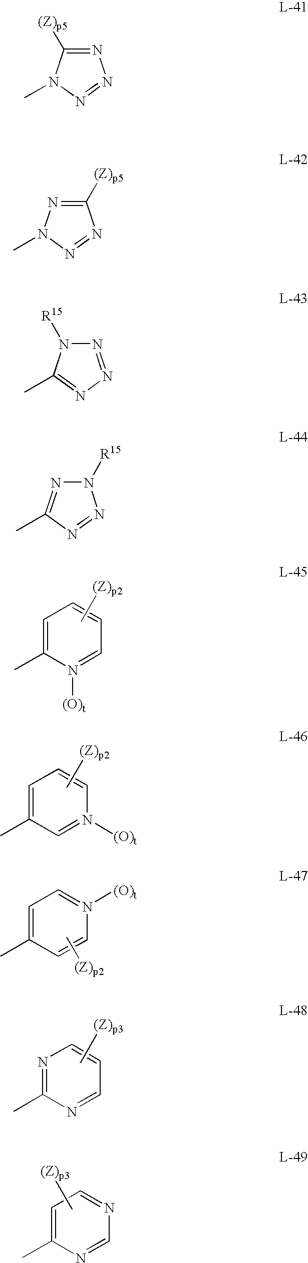 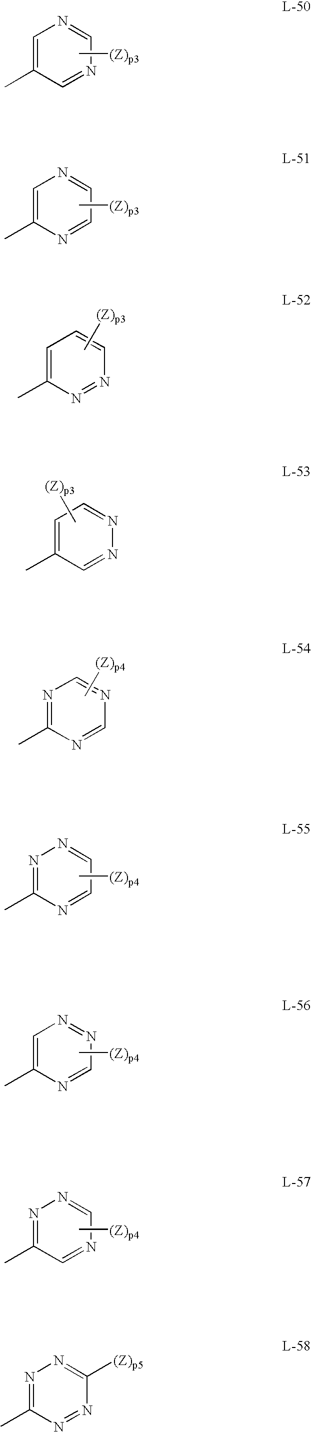
M represents an aromatic heterocyclic ring represented by any of the formula M-1 to the formula M-28, 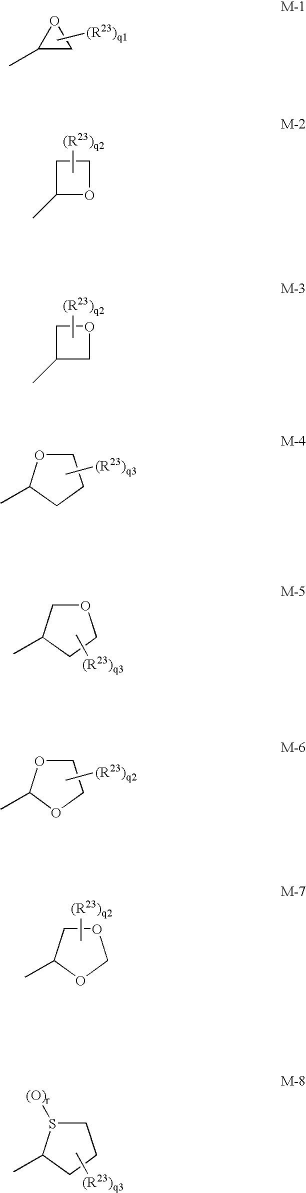 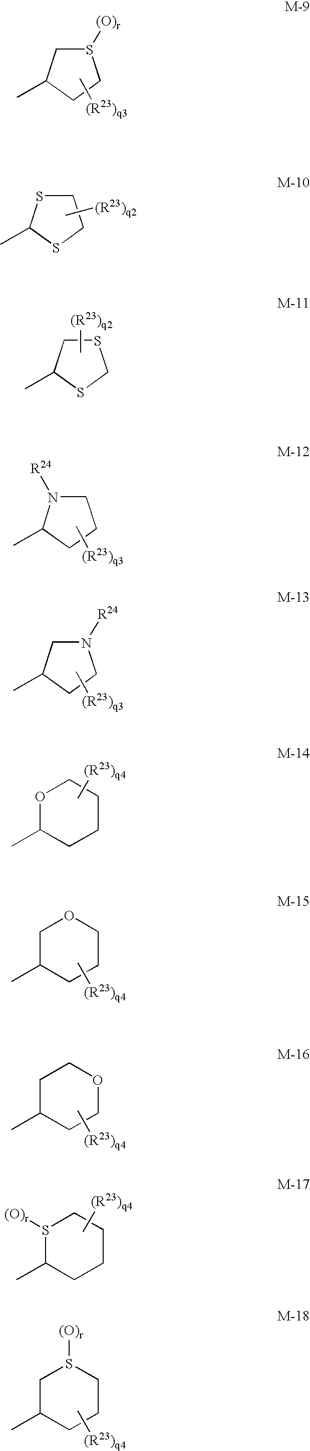 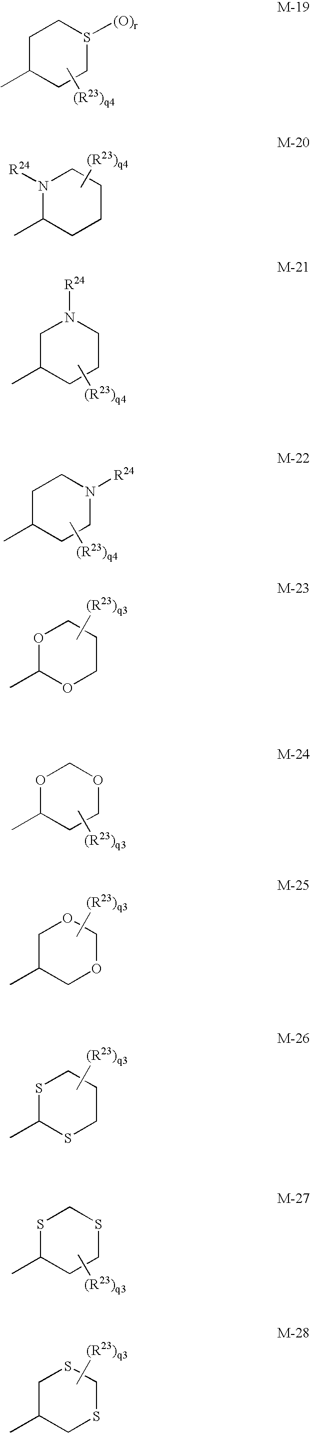
Z represents a halogen atom, cyano, nitro, azide, —SCN, —SF₅, a C₁ to C₆ alkyl, a C₁ to C₆ haloalkyl, a C₁ to C₃ alkoxy(C₁ to C₃) alkyl, a C₁ to C₃ haloalkoxy(C₁ to C₃) alkyl, a cyano(C₁ to C₆) alkyl, a hydroxy(C₁ to C₃) haloalkyl, a C₁ to C₃ alkoxy(C₁ to C₃) haloalkyl, a C₁ to C₃ haloalkoxy(C₁ to C₃) haloalkyl, a C₁ to C₃ alkylthio (C₁ to C₃) alkyl, a C₁ to C₃ haloalkylthio (C₁ to C₃) alkyl, a C₁ to C₃ alkylsulfinyl(C₁ to C₃) alkyl, a C₁ to C₃ haloalkyl-sulfinyl(C₁ to C₃) alkyl, a C₁ to C₃ alkylsulfonyl(C₁ to C₃) alkyl, a C₁ to C₃ haloalkylsulfonyl-(C₁ to C₃) alkyl, a C₃ to C₈ cycloalkyl, a C₃ to C₈ halocycloalkyl, a C₂ to C₆ alkenyl, a C₂ to C₆ haloalkenyl, a C₃ to C₈ cycloalkenyl, a C₃ to C₈ halocycloalkenyl, a C₂ to C₆ alkynyl, a C₂ to C₆ haloalkynyl, —OH, a C₁ to C₆ alkoxy, a C₁ to C₆ haloalkoxy, a C₁ to C₃ haloalkoxy-(C₁ to C₃) haloalkoxy, a C₂ to C₆ alkenyloxy, a C₂ to C₆ haloalkenyloxy, a C₃ to C₆ alkynyl-oxy, a C₃ to C₆ haloalkynyloxy, a C₁ to C₆ alkylsulfonyloxy, a C₁ to C₆ haloalkylsulfonyloxy, —SH, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl, a C₁ to C₆ haloalkylsulfonyl, —NH₂, a C₁ to C₆ alkylamino, a di(C₁ to C₆ alkyl)amino, a C₁ to C₆ alkylsulfonylamino, a C₁ to C₆ haloalkyl-sulfonylamino, a C₁ to C₆ alkoxycarbonyl, a C₁ to C₆ haloalkoxycarbonyl, —C(O)NH₂, a C₁ to C₆ alkylaminocarbonyl, a di(C₁ to C₆ alkyl)aminocarbonyl, —C(S)NH₂, a C₁ to C₆ alkylaminosulfonyl, a di(C₁ to C₆ alkyl)aminosulfonyl or a tri(C₁ to C₆ alkyl)silyl, when p1, p2, p3 or p4 is an integer of 2 or more, each Z may be the same or different from each other,
further, when two Zs are adjacent to each other, the adjacent two Zs may form a 5-membered ring or 6-membered ring with the carbon atoms to which two Zs are bonded by forming —CH₂CH₂CH₂—, —CH₂CH₂O—, —CH₂OCH₂—, —OCH₂O—, —CH₂CH₂S—,
—CH₂SCH₂—, —CH₂CH₂CH₂CH₂—, —CH₂CH₂CH₂O—, —CH₂CH₂OCH₂—, —CH₂OCH₂O—, —OCH₂CH₂O—, —OCH₂CH₂S— or —CH═CHCH═CH—, and at this time, each hydrogen atom bonded to the respective carbon atoms which form the ring may be optionally substituted by a halogen atom or a C₁ to C₆ alkyl group,
R⁷ represents a halogen atom, cyano, a C₃ to C₈ cycloalkyl, a C₃ to C₈ halocycloalkyl, —OH, —OR⁸, —SH, —S(O)ᵣR⁸, —N(R¹⁰)R⁹, —N(R¹⁰)CHO, —N(R¹⁰)C(O)R⁹, —N(R¹⁰)C(O)OR⁹, —N(R¹⁰)C(O)SR⁹, —N(R¹⁰)C(S)OR⁹, —N(R¹⁰)C(S)SR⁹, —N(R¹⁰)S(O)₂R⁹,
—C(O)OR⁹, —C(O)N(R¹⁰)R⁹, —Si(R¹³)(R¹⁴)R¹², phenyl, a phenyl substituted by (Z)ₚ₁, L or M,
R⁸ represents a C₁ to C₆ alkyl, a (C₁ to C₆) alkyl optionally substituted by R²⁵, a C₃ to C₈ cycloalkyl, a (C₃ to C₈) cycloalkyl optionally substituted by R²⁵, a C₂ to C₆ alkenyl, a (C₂ to C₆) alkenyl optionally substituted by R²⁵, a C₃ to C₈ cycloalkenyl, a C₃ to C₈ halocycloalkenyl, a C₃ to C₆ alkynyl, a (C₃ to C₆) alkynyl optionally substituted by R²⁵, phenyl, a phenyl substituted by (Z)ₚ₁, L or M,
R⁹ represents a C₁ to C₆ alkyl, a C₁ to C₆ haloalkyl, a C₃ to C₆ cycloalkyl (C₁ to C₄) alkyl, a C₁ to C₆ alkoxy(C₁ to C₄) alkyl, a C₁ to C₆ alkylthio (C₁ to C₄) alkyl, a cyano(C₁ to C₆) alkyl, a phenyl(C₁ to C₄) alkyl, a phenyl(C₁ to C₄) alkyl substituted by (Z)ₚ₁, an L-(C₁ to C₄) alkyl, an M-(C₁ to C₄) alkyl, a C₃ to C₈ cycloalkyl, a C₃ to C₈ halocycloalkyl, a C₃ to C₆ alkenyl, a C₃ to C₆ halo-alkenyl, a C₃ to C₆ alkynyl, phenyl or a phenyl substituted by (Z)ₚ₁,
R¹⁰ represents a hydrogen atom or a C₁ to C₆ alkyl, or R⁹ and R¹⁰ are combined in combination to form a C₂ to C₆ alkylene chain whereby they may form a 3 to 7-membered ring with an atom(s) to which they are bonded, and at this time, the alkylene chain may contain one oxygen atom, sulfur atom or nitrogen atom, and may be optionally substituted by a halogen atom, a C₁ to C₆ alkyl group, a C₁ to C₆ alkoxy group, a formyl group, a C₁ to C₆ alkylcarbonyl group or a C₁ to C₆ alkoxycarbonyl group,
R¹¹ represents a hydrogen atom, a C₁ to C₆ alkyl, a C₁ to C₆ haloalkyl, a phenyl(C₁ to C₄) alkyl, a phenyl(C₁ to C₄) alkyl substituted by (Z)ₚ₁, a C₃ to C₆ alkenyl, a C₃ to C₆ haloalkenyl, a C₃ to C₆ alkynyl or a C₃ to C₆ haloalkynyl, or R¹¹ is combined with R⁹ to form a C₂ to C₄ alkylene chain whereby it may form a 5 to 7-membered ring with an atom(s) to which they are bonded, and at this time, the alkylene chain may contain one oxygen atom, sulfur atom or nitrogen atom, and may be optionally substituted by a halogen atom or a C₁ to C₆ alkyl group,
R¹² represents a C₁ to C₆ alkyl, a C₁ to C₆ haloalkyl, a C₃ to C₆ alkenyl, phenyl or a phenyl substituted by (Z)ₚ₁,
R¹³ and R¹⁴ each independently represent a C₁ to C₆ alkyl or a C₁ to C₆ haloalkyl,
R¹⁵ represents a hydrogen atom, a C₁ to C₆ alkyl, a C₁ to C₆ haloalkyl, a C₁ to C₆ alkoxycarbonyl(C₁ to C₄) alkyl, a C₁ to C₆ haloalkoxycarbonyl(C₁ to C₄) alkyl, a phenyl(C₁ to C₄) alkyl, a phenyl(C₁ to C₄) alkyl substituted by (Z)ₚ₁, a C₃ to C₆ alkenyl, a C₃ to C₆ haloalkenyl, a C₃ to C₆ alkynyl, a C₃ to C₆ haloalkynyl, a C₁ to C₆ alkoxy, a C₁ to C₆ alkoxycarbonyl, a C₁ to C₆ haloalkoxycarbonyl, phenyl or a phenyl substituted by (Z)ₚ₁,
R¹⁶ represents a halogen atom, cyano, nitro, a C₃ to C₈ cycloalkyl, a C₃ to C₈ halocycloalkyl, —OR²⁶, —N(R²⁷)R²⁶, —SH, S(O)ᵣR²⁸, —SO₂NHR³⁰, —SO₂N(R³⁰)R²⁹, —CHO, —C(O)R²⁹, —C(O)OH, —C(O)OR²⁹, —C(O)SR²⁹, —C(O)NHR³⁰, —C(O)N(R³⁰)R²⁹, —C(O)C(O)OR²⁹,
—C(R³²)═NOH, —C(R³²)═NOR³¹, —Si(R¹³)(R¹⁴)R¹², —P(O)(OR²²)₂, —P(S)(OR²²)₂, —P(phenyl)₂,
—P(O)(phenyl)₂, phenyl, a phenyl substituted by (Z)ₚ₁, L or M,
R¹⁷ represents a C₁ to C₁₂ alkyl, a C₁ to C₁₂ haloalkyl, a C₁ to C₁₂ alkoxy(C₁ to C₁₂) alkyl, a cyano(C₁ to C₁₂) alkyl, a C₁ to C₁₂ alkoxycarbonyl(C₁ to C₁₂) alkyl, a phenyl(C₁ to C₄) alkyl, a phenyl(C₁ to C₄) alkyl substituted by (Z)ₚ₁, a C₃ to C₁₂ alkenyl, a C₃ to C₁₂ haloalkenyl, a C₃ to C₁₂ alkynyl, a C₃ to C₁₂ haloalkynyl, a C₁ to C₁₂ alkylcarbonyl, a C₁ to C₁₂ alkoxycarbonyl, phenyl or a phenyl substituted by (Z)ₚ₁,
R¹⁸ represents a C₁ to C₁₂ alkyl, a C₁ to C₁₂ haloalkyl, a C₁ to C₁₂ alkoxy(C₁ to C₁₂) alkyl, a cyano(C₁ to C₁₂) alkyl, a C₁ to C₁₂ alkoxycarbonyl(C₁ to C₁₂) alkyl, a phenyl(C₁ to C₄) alkyl, a phenyl(C₁ to C₄) alkyl substituted by (Z)ₚ₁, a C₃ to C₁₂ alkenyl, a C₃ to C₁₂ haloalkenyl, a C₃ to C₁₂ alkynyl, a C₃ to C₁₂ haloalkynyl, phenyl or a phenyl substituted by (Z)ₚ₁, or R¹⁷ and R¹⁸ are combined in combination to form a C₄ to C₇ alkylene chain whereby it may form a 5 to 8-membered ring with the nitrogen atom to which they are bonded, and at this time, the alkylene chain may contain one oxygen atom or sulfur atom, and may be optionally substituted by a C₁ to C₄ alkyl group or a C₁ to C₄ alkoxy group,
R¹⁹ represents a hydrogen atom, a C₁ to C₆ alkyl, a C₁ to C₆ haloalkyl, a phenyl(C₁ to C₄) alkyl, a phenyl(C₁ to C₄) alkyl substituted by (Z)ₚ₁, a C₃ to C₆ alkenyl, a C₃ to C₆ haloalkenyl, a C₃ to C₆ alkynyl, —CHO, a C₁ to C₆ alkylcarbonyl, a C₁ to C₆ haloalkylcarbonyl, a C₁ to C₆ alkoxycarbonyl, a C₁ to C₆ haloalkoxycarbonyl, a phenyl(C₁ to C₄) alkoxycarbonyl, a phenyl(C₁ to C₄) alkoxycarbonyl substituted by (Z)ₚ₁, phenoxycarbonyl, a phenoxycarbonyl substituted by (Z)ₚ₁, phenylcarbonyl, a phenylcarbonyl substituted by (Z)ₚ₁, phenyl or a phenyl substituted by (Z)ₚ₁,
R²⁰ represents a hydrogen atom, a C₁ to C₆ alkyl, —CHO, a C₁ to C₆ alkylcarbonyl, a C₁ to C₆ haloalkylcarbonyl or a C₁ to C₆ alkoxycarbonyl,
R²¹ represents cyano, a C₃ to C₈ cycloalkyl, a C₃ to C₈ halocycloalkyl, —OH, —OR⁸, —SH, —S(O)ᵣR⁸, —N(R¹⁰)R⁹, —N(R¹⁰)CHO, —N(R¹⁰)C(O)R⁹, —N(R¹⁰)C(O)OR⁹, —N(R¹⁰)C(O)SR⁹,
—N(R¹⁰)C(S)OR⁹, —N(R¹⁰)C(S)SR⁹, —N(R¹⁰)S(O)₂R⁹, —C(O)OR⁹, —C(O)N(R¹⁰)R⁹, —Si(R¹³)(R¹⁴)R¹², phenyl, a phenyl substituted by (Z)ₚ₁, L or M,
R²² represents a C₁ to C₆ alkyl or a C₁ to C₆ haloalkyl,
R²³ represents a halogen atom, cyano, a C₁ to C₆ alkyl, a C₁ to C₆ haloalkyl, a hydroxy(C₁ to C₆) alkyl, a C₁ to C₄ alkoxy(C₁ to C₄) alkyl, a C₁ to C₄ alkoxycarbonyl(C₁ to C₄) alkyl, a C₁ to C₆ alkoxy, a C₁ to C₆ alkoxycarbonyl, phenyl or a phenyl substituted by (Z)ₚ₁, when q1, q2, q3 or q4 is an integer of 2 or more, each R²³ may be the same or different from each other,
R²⁴ represents a hydrogen atom, a C₁ to C₆ alkyl, a C₁ to C₆ haloalkyl, —CHO, a C₁ to C₆ alkylcarbonyl, a C₁ to C₆ haloalkylcarbonyl, a phenyl(C₁ to C₄) alkylcarbonyl, a phenyl(C₁ to C₄) alkylcarbonyl substituted by (Z)ₚ₁, a C₁ to C₆ alkoxycarbonyl, a C₁ to C₆ haloalkoxycarbonyl, a phenyl(C₁ to C₄) alkoxycarbonyl, a phenyl(C₁ to C₄) alkoxycarbonyl substituted by (Z)ₚ₁, a C₁ to C₆ alkylthio carbonyl, a C₁ to C₆ alkoxythiocarbonyl, a C₁ to C₆ alkylaminocarbonyl, a di(C₁ to C₆ alkyl)aminocarbonyl, a C₁ to C₆ alkylaminothiocarbonyl, a di(C₁ to C₆ alkyl)aminothiocarbonyl, phenylcarbonyl, a phenylcarbonyl substituted by (Z)ₚ₁, a C₁ to C₆ alkylsulfonyl, a C₁ to C₆ haloalkylsulfonyl, phenylsulfonyl, a phenylsulfonyl substituted by (Z)ₚ₁, —P(O)(OR²²)₂ or —P(S)(OR²²)₂,
R²⁵ represents a halogen atom, cyano, a C₃ to C₈ cycloalkyl, a C₃ to C₈ halocycloalkyl, a C₁ to C₆ alkoxy, a C₁ to C₆ haloalkoxy, a C₁ to C₆ alkylthio, a C₁ to C₆ haloalkylthio, a C₁ to C₆ alkylsulfonyl, a C₁ to C₆ haloalkylsulfonyl, a C₁ to C₆ alkylamino, a di(C₁ to C₆ alkyl)amino, —CHO, a C₁ to C₆ alkylcarbonyl, a C₁ to C₆ haloalkylcarbonyl, a C₁ to C₆ alkoxycarbonyl, a C₁ to C₆ haloalkoxycarbonyl, —CH═NOR¹¹, —C(R⁹)═NOR¹¹, phenyl, a phenyl substituted by (Z)ₚ₁, L or M,
R²⁶ represents a hydrogen atom, a C₁ to C₈ alkyl, a (C₁ to C₈) alkyl optionally substituted by R³³, a C₃ to C₈ cycloalkyl, a (C₃ to C₈) cycloalkyl optionally substituted by R³³, a C₃ to C₈ alkenyl, a (C₃ to C₈) alkenyl optionally substituted by R³³, a C₃ to C₈ alkynyl, a (C₃ to C₈) alkynyl optionally substituted by R³³, —CHO, —C(O)R²⁹, —C(O)OR²⁹, —C(O)SR²⁹,
—C(O)NHR³⁰, —C(O)N(R³⁰)R²⁹, —C(O)C(O)R²⁹, —C(O)C(O)OR²⁹, —C(S)R²⁹, —C(S)O R²⁹, —C(S)SR²⁹, —C(S)NHR³⁰, —C(S)N(R³⁰)R²⁹, —S(O)₂R²⁹, —S(O)₂N(R³⁰)R²⁹, —Si(R¹³)(R¹⁴)R¹²,
—P(O)(OR²²)₂, —P(S)(OR²²)₂, phenyl, a phenyl substituted by (Z)ₚ₁, L or M,
R²⁷ represents a hydrogen atom, a C₁ to C₆ alkyl, a C₁ to C₆ haloalkyl, a C₃ to C₆ cycloalkyl or a C₁ to C₆ alkoxy, or R²⁶ and R²⁷ are combined in combination to form a C₂ to C₅ alkylene chain whereby it forms a 3 to 6-membered ring with the nitrogen atom to which they are bonded, and at this time, the alkylene chain may contain one oxygen atom or sulfur atom, and may be substituted by a halogen atom, a C₁ to C₆ alkyl group, a C₁ to C₆ alkoxy group, a phenyl group or a phenyl group substituted by (Z)ₚ₁,
R²⁸ represents a C₁ to C₈ alkyl, a (C₁ to C₈) alkyl optionally substituted by R³³, a C₃ to C₈ cycloalkyl, a (C₃ to C₈) cycloalkyl optionally substituted by R³³, a C₃ to C₈ alkenyl, a (C₃ to C₈) alkenyl optionally substituted by R³³, a C₃ to C₈ alkynyl, a (C₃ to C₈) alkynyl optionally substituted by R³³, —SH, a C₁ to C₆ alkylthio, a C₁ to C₆ haloalkylthio, phenylthio, a phenylthio substituted by (Z)ₚ₁, —CHO, —C(O)R²⁹, —C(O)OR²⁹, —C(O)SR²⁹, —C(O)NHR³⁰,
—C(O)N(R³⁰)R²⁹, —C(O)C(O)R²⁹, —C(O)C(O)OR²⁹, —C(S)R²⁹, —C(S)OR²⁹, —C(S)SR²⁹, —C(S)NHR³⁰, —C(S)N(R³⁰)R²⁹, —P(O)(OR²²)₂, —P(S)(OR²²)₂, phenyl, a phenyl substituted by (Z)ₚ₁, L-18, L-21, L-25, L-30 to L-35, L-45, L-48, L-49 or M,
R²⁹ represents a C₁ to C₆ alkyl, a C₁ to C₆ haloalkyl, a C₃ to C₈ cycloalkyl (C₁ to C₄) alkyl, a C₁ to C₆ alkoxy(C₁ to C₄) alkyl, a C₁ to C₆ haloalkoxy(C₁ to C₄) alkyl, a C₁ to C₆ alkylthio (C₁ to C₄) alkyl, a C₁ to C₆ haloalkylthio (C₁ to C₄) alkyl, a C₁ to C₆ alkylsulfonyl(C₁ to C₄) alkyl, a C₁ to C₆ haloalkylsulfonyl(C₁ to C₄) alkyl, a cyano(C₁ to C₆) alkyl, a C₁ to C₆ alkylcarbonyl(C₁ to C₄) alkyl, a C₁ to C₆ haloalkylcarbonyl(C₁ to C₄) alkyl, a C₁ to C₆ alkoxycarbonyl(C₁ to C₄) alkyl, a di(C₁ to C₆ alkyl)aminocarbonyl(C₁ to C₄) alkyl, a tri(C₁ to C₄ alkyl)silyl (C₁ to C₄) alkyl, a phenyl(C₁ to C₄) alkyl, a phenyl(C₁ to C₄) alkyl substituted by (Z)ₚ₁, an L-(C₁ to C₄) alkyl, an M-(C₁ to C₄) alkyl, a C₃ to C₈ cycloalkyl, a C₃ to C₈ halocycloalkyl, a C₂ to C₆ alkenyl, a C₂ to C₆ haloalkenyl, a C₂ to C₆ alkynyl, a C₂ to C₆ haloalkynyl, phenyl, a phenyl substituted by (Z)ₚ₁, L or M, R³⁰ represents a hydrogen atom, a C₁ to C₆ alkyl, a C₁ to C₆ haloalkyl, phenyl or a phenyl substituted by (Z)ₚ₁, or R²⁹ and R³⁰ are combined to form a C₂ to C₅ alkylene chain whereby it may form a 3 to 6-membered ring with the nitrogen atom to which they are bonded, and at this time, the alkylene chain may contain one oxygen atom, sulfur atom or nitrogen atom, and may be optionally substituted by a halogen atom, a C₁ to C₆ alkyl group, a C₁ to C₆ alkoxy group, a formyl group, a C₁ to C₆ alkylcarbonyl group, a C₁ to C₆ alkoxycarbonyl group, a phenyl group or a phenyl group substituted by (Z)ₚ₁,
R³¹ represents a hydrogen atom, a C₁ to C₈ alkyl, a (C₁ to C₈) alkyl optionally substituted by R³³, a C₃ to C₈ cycloalkyl, a C₃ to C₈ alkenyl, a (C₃ to C₈) alkenyl optionally substituted by R³³, a C₃ to C₈ alkynyl or a (C₃ to C₈) alkynyl optionally substituted by R³³,
R³² represents a hydrogen atom, a C₁ to C₆ alkyl, a C₁ to C₆ haloalkyl, a C₃ to C₈ cycloalkyl (C₁ to C₄) alkyl, a C₁ to C₆ alkoxy(C₁ to C₄) alkyl, a C₁ to C₆ haloalkoxy(C₁ to C₄) alkyl, a C₁ to C₆ alkylthio (C₁ to C₄) alkyl, a C₁ to C₆ haloalkylthio (C₁ to C₄) alkyl, a C₁ to C₆ alkylsulfonyl(C₁ to C₄) alkyl, a C₁ to C₆ haloalkylsulfonyl(C₁ to C₄) alkyl, a phenyl(C₁ to C₄) alkyl, a phenyl(C₁ to C₄) alkyl substituted by (Z)ₚ₁, phenyl or a phenyl substituted by (Z)ₚ₁,
R³³ represents a halogen atom, cyano, nitro, a C₃ to C₈ cycloalkyl, a C₃ to C₈ halocycloalkyl, —OH, —OR³⁴, —SH, —S(O)ᵣR³⁴, —NHR³⁵, —N(R³⁵)R³⁴, —CHO, —C(O)R²⁹, —C(O)OR²⁹, —C(O)SR²⁹, —C(O)NHR³⁰, —C(O)N(R³⁰)R²⁹, —C(O)C(O)OR²⁹, —CH═NOR¹¹, —C(R⁹)═NOR¹¹, —Si(R¹³)(R¹⁴)R¹², —P(O)(OR²²)₂, —P(S)(OR²²)₂, —P(phenyl)₂, —P(O)(phenyl)₂, phenyl, a phenyl substituted by (Z)ₚ₁, L or M,
R³⁴ represents a C₁ to C₆ alkyl, a C₁ to C₆ haloalkyl, a C₁ to C₆ alkoxy(C₁ to C₄) alkyl, a C₁ to C₆ alkylthio(C₁ to C₄) alkyl, a phenyl(C₁ to C₄) alkyl, a phenyl(C₁ to C₄) alkyl substituted by (Z)ₚ₁, a C₃ to C₈ cycloalkyl, a C₃ to C₈ halocycloalkyl, a C₃ to C₆ alkenyl, a C₃ to C₆ haloalkenyl, a C₃ to C₈ cycloalkenyl, a C₃ to C₈ halocycloalkenyl, a C₃ to C₆ alkynyl, a C₃ to C₆ haloalkynyl, —CHO, a C₁ to C₆ alkylcarbonyl, a C₁ to C₆ haloalkylcarbonyl, a C₁ to C₆ alkoxycarbonyl, a C₁ to C₆ haloalkoxycarbonyl, a C₁ to C₆ alkylaminocarbonyl, a di(C₁ to C₆ alkyl) aminocarbonyl, phenylcarbonyl, a phenylcarbonyl substituted by (Z)ₚ₁, a C₁ to C₆ alkylaminothiocarbonyl, a di(C₁ to C₆ alkyl) aminothiocarbonyl, phenyl, a phenyl substituted by (Z)ₚ₁, L or M,
R³⁵ represents a hydrogen atom, a C₁ to C₆ alkyl, a C₁ to C₆ haloalkyl, a C₃ to C₈ cycloalkyl, a C₃ to C₆ alkenyl, a C₃ to C₆ alkynyl, a C₁ to C₆ alkylcarbonyl, a C₁ to C₆ haloalkylcarbonyl, a C₁ to C₆ alkoxycarbonyl, a C₁ to C₆ haloalkoxycarbonyl, phenoxycarbonyl, a phenoxycarbonyl substituted by (Z)ₚ₁, phenylcarbonyl, a phenylcarbonyl substituted by (Z)ₚ₁, a C₁ to C₆ alkylsulfonyl, a C₁ to C₆ haloalkylsulfonyl, phenyl, a phenyl substituted by (Z)ₚ₁, L or M, or R³⁴ and R³⁵ are combined to form a C₂ to C₅ alkylene chain, whereby it may form a 3 to 6-membered ring with the nitrogen atom to which they are bonded, and at this time, the alkylene chain may contain one oxygen atom or sulfur atom, and may be substituted by a halogen atom or a methyl group,
m is an integer of 0 to 4,
n is an integer of 0 to 4,
p1 is an integer of 1 to 5,
p2 is an integer of 0 to 4,
p3 is an integer of 0 to 3,
p4 is an integer of 0 to 2,
p5 is an integer of 0 or 1,
q1 is an integer of 0 to 3,
q2 is an integer of 0 to 5,
q3 is an integer of 0 to 7,
q4 is an integer of 0 to 9,
r is an integer of 0 to 2, t is an integer of 0 or 1,
or a salt thereof.

2. The substituted benzanilide compound according to claim 1, wherein W¹ and W² each represent an oxygen atom,
X represents a halogen atom, cyano, nitro, a C₁ to C₆ alkyl, a C₁ to C₆ haloalkyl, a C₁ to C₆ alkoxy, a C₁ to C₆ haloalkoxy, a C₁ to C₆ alkylsulfonyloxy, a C₁ to C₆ haloalkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl or a C₁ to C₆ haloalkylsulfonyl, and when m is 2 or 3, each X may be the same or different from each other, and when two Xs are adjacent to each other, the adjacent two Xs may form a 5-membered ring or 6-membered ring with the carbon atoms to which two Xs are bonded by forming —OCH₂O— or —OCH₂CH₂O—, and at this time, the hydrogen atom(s) bonded to the respective carbon atoms which form a ring may be optionally replaced with a halogen atom, a C₁ to C₄ alkyl group or a C₁ to C₄ haloalkyl group,
Y represents a halogen atom, a C₁ to C₆ alkyl, a C₁ to C₆ haloalkyl, a hydroxy(C₁ to C₆) alkyl, a C₁ to C₃ alkoxy(C₁ to C₃) alkyl, a C₁ to C₆ alkoxy, a C₁ to C₆ haloalkoxy, a C₁ to C₆ alkylthio, a C₁ to C₆ haloalkylthio, a C₁ to C₆ alkylamino or a di(C₁ to C₆ alkyl)amino, when n is 2 or 3, each Y may be the same or different from each other,
R¹ represents a C₁ to C₈ alkyl, a (C₁ to C₈) alkyl optionally substituted by R¹⁶, a C₃ to C₈ cycloalkyl, a C₃ to C₈ alkenyl, a C₃ to C₈ alkynyl, a C₁ to C₈ alkoxy, M-4, M-5, M-8, M-9, M-13 to M-19, M-21 or M-22,
R² and R³ each independently represent a hydrogen atom, a C₁ to C₆ alkyl, a C₁ to C₄ alkoxy(C₁ to C₄) alkyl, a C₁ to C₄ alkylthio (C₁ to C₄) alkyl, a C₁ to C₄ alkylsulfonyl(C₁ to C₄) alkyl, a C₃ to C₆ alkenyl, a C₃ to C₆ alkynyl, a C₁ to C₆ alkylthio, a C₁ to C₆ haloalkylthio, phenylthio, a phenylthio substituted by (Z)ₚ₁ or —SN(R¹⁸)R¹⁷, or R² and R¹ may be combined to form a C₂ to C₆ alkylene chain whereby they may form a 3 to 7-membered ring with the nitrogen atom to which they are bonded, and at this time, the alkylene chain may contain one oxygen atom or sulfur atom,
R⁴ represents a C₁ to C₆ alkyl, a C₁ to C₆ haloalkyl, a (C₁ to C₆) alkyl optionally substituted by R²¹, a (C₁ to C₆) haloalkyl optionally substituted by R²¹, a C₃ to C₈ cycloalkyl, a C₃ to C₈ halocycloalkyl, a C₃ to C₆ haloalkenyl, a C₃ to C₆ haloalkynyl, phenyl or a phenyl substituted by (Z)ₚ₁,
R⁵ represents cyano, a (C₁ to C₆) alkyl optionally substituted by R²¹, a (C₁ to C₆) haloalkyl optionally substituted by R²¹, a C₃ to C₈ cycloalkyl, a C₃ to C₈ halocycloalkyl, a (C₂ to C₆) alkenyl optionally substituted by R²¹, a (C₂ to C₆) alkynyl optionally substituted by R²¹, —C(O)OR⁹, —C(O)SR⁹, —C(O)NHR¹⁰, —C(O)N(R¹⁰)R⁹, —C(S)OR⁹, —C(S)SR⁹,
—C(S)NHR¹⁰, —C(S)N(R¹⁰)R⁹, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-25 to L-35, L-37, L-38, L-40, L-43 to L-58, M-4, M-5, M-8, M-9, M-14 to M-18 or M-19, or may be combined with Y existing at the adjacent position to form a C₂ to C₃ alkylene chain, whereby it may form a 5 to 6-membered ring which fuses with a benzene ring, and at this time, the alkylene chain may contain one oxygen atom, sulfur atom or nitrogen atom, and may be optionally substituted by a halogen atom or a C₁ to C₆ haloalkyl group,
R⁶ represents a hydrogen atom, a C₁ to C₆ alkyl, a C₁ to C₆ haloalkyl, a C₁ to C₄ alkoxy(C₁ to C₄) alkyl, a C₁ to C₄ alkylthio(C₁ to C₄) alkyl, a cyano(C₁ to C₆) alkyl, a phenyl(C₁ to C₄) alkyl, a phenyl(C₁ to C₄) alkyl substituted by (Z)ₚ₁, a C₃ to C₆ alkenyl, a C₃ to C₆ haloalkenyl, a phenyl(C₃ to C₆) alkenyl, a phenyl(C₃ to C₆) alkenyl substituted by (Z)ₚ₁, a C₃ to C₆ alkynyl, a C₃ to C₆ haloalkynyl, a phenyl(C₃ to C₆) alkynyl, a phenyl(C₃ to C₆) alkynyl substituted by (Z)ₚ₁, —S(O)₂R⁹, —C(O)R⁹, —C(O)NHR¹⁰, —C(O)N(R¹⁰)R⁹, —C(S)NHR¹⁰,
—C(S)N(R¹⁰)R⁹, —Si(R¹³)(R¹⁴)R¹², —P(O)(OR²²)₂ or —P(S)(OR²²)₂,
Z represents a halogen atom, cyano, nitro, a C₁ to C₆ alkyl, a C₁ to C₆ haloalkyl, a C₁ to C₃ alkylthio(C₁ to C₃) alkyl, a C₁ to C₃ haloalkylthio(C₁ to C₃) alkyl, a C₁ to C₃ alkylsulfinyl(C₁ to C₃) alkyl, a C₁ to C₃ haloalkylsulfinyl(C₁ to C₃) alkyl, a C₁ to C₃ alkylsulfonyl(C₁ to C₃) alkyl, a C₁ to C₃ haloalkylsulfonyl(C₁ to C₃) alkyl, a C₁ to C₆ alkoxy, a C₁ to C₆ haloalkoxy, a C₁ to C₃ haloalkoxy(C₁ to C₃) haloalkoxy, a C₁ to C₆ alkylsulfonyloxy, a C₁ to C₆ haloalkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl, a C₁ to C₆ haloalkylsulfonyl, —C(O)NH₂ or —C(S)NH₂, and when p1, p2, p3 or p4 is an integer of 2 or more, each Z may be the same or different from each other,
further, when two Zs are adjacent to each other, the adjacent two Zs may form a 5-membered ring or 6-membered ring with the carbon atoms to which two Zs are bonded by forming —CF₂CF₂O—, —CF₂OCF₂—, —OCF₂O—, —OCF₂CHFO—, —OCF₂CF₂O— or —CH═CHCH═CH—,
R⁹ represents a C₁ to C₆ alkyl, a C₁ to C₆ haloalkyl, a C₃ to C₆ cycloalkyl(C₁ to C₄) alkyl, a phenyl(C₁ to C₄) alkyl, a phenyl(C₁ to C₄) alkyl substituted by (Z)ₚ₁, a C₃ to C₈ cycloalkyl, a C₃ to C₈ halocycloalkyl, phenyl or a phenyl substituted by (Z)ₚ₁,
R¹⁰ represents a hydrogen atom or a C₁ to C₆ alkyl, or R⁹ and R¹⁰ are combined to form a C₄ to C₅ alkylene chain, whereby it may form a 5-membered ring or 6-membered ring with the nitrogen atom to which they are bonded, and at this time, the alkylene chain may contain one oxygen atom or sulfur atom,
R¹² represents a C₁ to C₆ alkyl, phenyl or a phenyl substituted by (Z)ₚ₁,
R¹³ and R¹⁴ each independently represent a C₁ to C₆ alkyl,
R¹⁵ represents a C₁ to C₆ alkyl, a C₁ to C₆ haloalkyl, a phenyl(C₁ to C₄) alkyl, a phenyl(C₁ to C₄) alkyl substituted by (Z)ₚ₁, phenyl or a phenyl substituted by (Z)ₚ₁,
R¹⁶ represents a halogen atom, cyano, a C₃ to C₆ cycloalkyl, —OR²⁶, —N(R²⁷)R²⁶,
—S(O)ᵣR²⁸, —SO₂N(R³⁰)R²⁹, a C₁ to C₆ alkoxycarbonyl, —C(O)N(R³⁰)R²⁹, —C(R³²)═NOH, —C(R³²)═NOR³¹, —Si(R¹³)(R¹⁴)R¹², phenyl, a phenyl substituted by (Z)ₚ₁, L-1, L-2, L-3, L-4, L-45, L-46, L-47 or M,
R¹⁷ represents a C₁ to C₆ alkyl, a C₁ to C₆ alkoxycarbonyl(C₁ to C₄) alkyl or a C₁ to C₆ alkoxycarbonyl,
R¹⁸ represents a C₁ to C₆ alkyl, or R¹⁷ and R¹⁸ are combined to form a C₄ to C₅ alkylene chain whereby it may form a 5-membered ring or 6-membered ring with the nitrogen atom to which they are bonded, and at this time, the alkylene chain may contain one oxygen atom or sulfur atom, and may be optionally substituted by a methyl group or a methoxy group,
R²¹ represents cyano, a C₃ to C₆ cycloalkyl, a C₃ to C₆ halocycloalkyl, a C₁ to C₆ alkoxy, a C₁ to C₆ haloalkoxy, phenoxy, a phenoxy substituted by (Z)ₚ₁, a C₁ to C₆ alkylthio, a C₁ to C₆ haloalkylthio, phenylthio, a phenylthio substituted by (Z)ₚ₁, a C₁ to C₆ alkylsulfinyl, a C₁ to C₆ haloalkylsulfinyl, a C₁ to C₆ alkylsulfonyl, a C₁ to C₆ haloalkylsulfonyl, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53,
R²² represents a C₁ to C₆ alkyl,
R²³ represents a C₁ to C₄ alkyl, when q1, q2, q3 or q4 is an integer of 2 or more, each R²³ may be the same or different from each other,
R²⁴ represents —CHO, a C₁ to C₆ alkylcarbonyl, a C₁ to C₆ alkoxycarbonyl or a C₁ to C₆ alkylsulfonyl,
R²⁶ represents a hydrogen atom, a C₁ to C₆ alkyl, a C₁ to C₆ haloalkyl, a C₁ to C₄ alkoxy(C₁ to C₄) alkyl, a C₁ to C₄ alkylthio(C₁ to C₄) alkyl, a phenyl(C₁ to C₄) alkyl, a phenyl(C₁ to C₄) alkyl substituted by (Z)ₚ₁, a C₁ to C₆ alkylcarbonyl, a C₁ to C₆ haloalkylcarbonyl, a C₃ to C₆ cycloalkylcarbonyl, a C₁ to C₆ alkoxycarbonyl, —C(O)N(R³⁰)R²⁹, a C₁ to C₆ alkylsulfonyl, a di(C₁ to C₆ alkyl)aminosulfonyl, phenylsulfonyl, a phenylsulfonyl substituted by (Z)ₚ₁, a di(C₁ to C₆ alkyl)phosphoryl, a di(C₁ to C₆ alkyl)thiophosphoryl, a tri(C₁ to C₄ alkyl)silyl, phenyl or a phenyl substituted by (Z)ₚ₁,
R²⁷ represents a hydrogen atom or a C₁ to C₆ alkyl,
R²⁸ represents a C₁ to C₆ alkyl, a C₁ to C₆ haloalkyl, a hydroxy(C₁ to C₄) alkyl, a C₁ to C₄ alkoxy(C₁ to C₄) alkyl, a C₁ to C₄ alkylthio(C₁ to C₄) alkyl, a C₁ to C₄ alkylcarbonyl(C₁ to C₄) alkyl, a C₁ to C₄ alkoxycarbonyl(C₁ to C₄) alkyl, a C₁ to C₄ alkylaminocarbonyl(C₁ to C₄) alkyl, a di(C₁ to C₄ alkyl)aminocarbonyl(C₁ to C₄) alkyl, a tri(C₁ to C₄ alkyl)silyl (C₁ to C₄) alkyl, a phenyl(C₁ to C₄) alkyl, a phenyl(C₁ to C₄) alkyl substituted by (Z)ₚ₁, a C₃ to C₆ alkenyl, a C₃ to C₆ alkynyl, a C₁ to C₆ alkylthio, phenyl, a phenyl substituted by (Z)ₚ₁, L-21, L-35, L-45 or L-48,
R²⁹ represents a C₁ to C₆ alkyl, a C₁ to C₄ alkoxy(C₁ to C₄) alkyl, a C₁ to C₄ alkylthio(C₁ to C₄) alkyl, a phenyl(C₁ to C₄) alkyl, a phenyl(C₁ to C₄) alkyl substituted by (Z)ₚ₁, a C₃ to C₆ cycloalkyl, a C₃ to C₆ alkenyl, a C₂ to C₆ alkynyl, phenyl or a phenyl substituted by (Z)ₚ₁,
R³⁰ represents a hydrogen atom or a C₁ to C₆ alkyl, or R²⁹ and R³⁰ are combined to form a C₂ to C₅ alkylene chain, whereby it may form a 3 to 6-membered ring with the nitrogen atom to which they are bonded, and at this time, the alkylene chain may contain one oxygen atom or sulfur atom,
R³¹ represents a C₁ to C₆ alkyl, a phenyl(C₁ to C₄) alkyl or a phenyl(C₁ to C₄) alkyl substituted by (Z)ₚ₁,
R³² represents a hydrogen atom or a C₁ to C₆ alkyl,
m is an integer of 0 to 3,
n is an integer of 0 to 3,
q2, q3 and q4 are each independently an integer of 0 to 2 or a salt thereof.

3. The substituted benzanilide compound according to claim 2, wherein X represents a halogen atom, nitro, a C₁ to C₆ alkyl, a C₁ to C₆ haloalkyl, a C₁ to C₆ alkoxy, a C₁ to C₆ haloalkoxy, a C₁ to C₆ alkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl or a C₁ to C₆ haloalkylsulfonyl, and when m is 2, each X may be the same or different from each other,
Y represents a halogen atom, a C₁ to C₆ alkyl, a C₁ to C₆ haloalkyl, a C₁ to C₆ alkoxy or a C₁ to C₆ alkylthio, and when n is 2, each Y may be the same or different from each other,
R¹ represents a C₁ to C₈ alkyl, a (C₁ to C₈) alkyl optionally substituted by R¹⁶, a C₃ to C₈ alkenyl or a C₃ to C₈ alkynyl,
R² represents a hydrogen atom or a C₁ to C₆ alkyl,
R³ represents a hydrogen atom,
R⁴ represents a C₁ to C₆ alkyl, a C₁ to C₆ haloalkyl, a C₁ to C₃ alkoxy(C₁ to C₃) haloalkyl, a C₁ to C₃ alkylthio(C₁ to C₃) haloalkyl, a C₃ to C₆ cycloalkyl or a C₃ to C₆ halocycloalkyl,
R⁵ represents a (C₁ to C₆) alkyl optionally substituted by R²¹, a (C₁ to C₆) haloalkyl optionally substituted by R²¹, a (C₂ to C₆) alkenyl optionally substituted by R²¹, a (C₂ to C₆) alkynyl optionally substituted by R²¹, a C₁ to C₆ alkoxycarbonyl, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 or L-53, or may be combined with Y existing at the adjacent position to form a C₂ to C₃ alkylene chain, whereby it may form a 5 to 6-membered ring which fuses with a benzene ring, and at this time, the alkylene chain may contain one oxygen atom, sulfur atom or nitrogen atom, and may be optionally substituted by a halogen atom,
R⁶ represents a hydrogen atom, a C₁ to C₆ alkyl, a C₁ to C₄ alkoxy(C₁ to C₄) alkyl, a C₁ to C₆ alkylcarbonyl or a tri(C₁ to C₄ alkyl)silyl,
R¹⁶ represents —OR²⁶, —N(R²⁷)R²⁶, —S(O)ᵣR²⁸, —SO₂N(R³⁰)R²⁹, —C(R³²)═NOH or —C(R³²)═NOR³¹,
R²¹ represents a C₁ to C₆ alkoxy, a C₁ to C₆ haloalkoxy, phenoxy, a phenoxy substituted by (Z)ₚ₁, phenylthio, a phenylthio substituted by (Z)ₚ₁, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53,
R²⁶ represents a hydrogen atom, a C₁ to C₈ alkyl, a C₁ to C₆ alkylcarbonyl, a C₁ to C₆ alkoxycarbonyl, a C₁ to C₆ alkylaminocarbonyl, a di(C₁ to C₆ alkyl)aminocarbonyl or a C₁ to C₆ alkylsulfonyl,
R²⁸ represents a C₁ to C₆ alkyl,
R²⁹ represents a C₁ to C₆ alkyl,
R³⁰ represents a hydrogen atom or a C₁ to C₆ alkyl,
R³¹ represents a C₁ to C₆ alkyl,
R³² represents a hydrogen atom,
m is an integer of 0 to 2,
n is an integer of 0 to 2
or a salt thereof.

4. The substituted benzanilide compound according to claim 3, wherein X represents a halogen atom, nitro, a C₁ to C₄ alkyl, a C₁ to C₄ haloalkyl, a C₁ to C₄ alkylthio, a C₁ to C₄ alkylsulfinyl or a C₁ to C₄ alkylsulfonyl, and when m is 2, each X may be the same or different from each other,
Y represents a halogen atom or a C₁ to C₄ alkyl, when n is 2, each Y may be the same or different from each other,
R¹ represents a C₁ to C₈ alkyl, a C₁ to C₄ alkylthio(C₁ to C₄) alkyl, a C₁ to C₄ alkylsulfinyl(C₁ to C₄) alkyl or a C₁ to C₄ alkylsulfonyl(C₁ to C₄) alkyl,
R² represents a hydrogen atom,
R⁴ represents a C₁ to C₆ alkyl or a C₁ to C₆ haloalkyl,
R⁵ represents phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 or L-53,
R⁶ represents a hydrogen atom
or a salt thereof.

5. An N-substituted phenyl-3-nitrophthalimide or substituted aniline represented by the formula (2) or the formula (3): 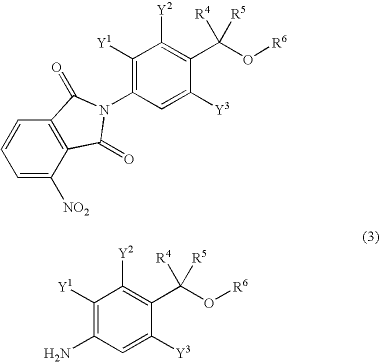
wherein Y¹ represents a hydrogen atom, a halogen atom, a C₁ to C₆ alkyl, a C₁ to C₆ haloalkyl, a C₁ to C₆ alkoxy or a C₁ to C₆ alkylthio,
Y² and Y³ each independently represent a hydrogen atom, or may form a C₂ to C₃ alkylene chain in combination with R⁵, whereby it may form a 5 to 6-membered ring which fuses with a benzene ring, at this time, the alkylene chain may contain one oxygen atom, sulfur atom or nitrogen atom, and may be optionally substituted by a halogen atom,
R⁴ represents a C₁ to C₆ alkyl, a C₁ to C₆ haloalkyl, a C₁ to C₃ alkoxy(C₁ to C₃) haloalkyl, a C₁ to C₃ alkylthio(C₁ to C₃) haloalkyl, a C₃ to C₆ cycloalkyl or a C₃ to C₆ halocycloalkyl,
R⁵ represents a (C₁ to C₆) alkyl optionally substituted by R²¹, a (C₁ to C₆) haloalkyl optionally substituted by R²¹, a (C₂ to C₆) alkenyl optionally substituted by R²¹, a (C₂ to C₆) alkynyl optionally substituted by R²¹, a C₁ to C₆ alkoxycarbonyl, phenyl, a phenyl substituted by (Z)ₚ₁, a phenoxyphenyl substituted by (Z)ₚ₁, a pyridyloxyphenyl substituted by (Z)ₚ₁, L-1 to L-4, L-8 to L-13, L-15 to L-23, L-45 to L-52 or L-53,
R⁶ represents a hydrogen atom, a C₁ to C₆ alkyl, a C₁ to C₄ alkoxy(C₁ to C₄) alkyl, a C₁ to C₆ alkylcarbonyl or a tri(C₁ to C₄ alkyl)silyl,
R²¹ represents a C₁ to C₆ alkoxy, a C₁ to C₆ haloalkoxy, phenoxy, a phenoxy substituted by (Z)ₚ₁, phenylthio, a phenylthio substituted by (Z)ₚ₁, phenylsulfonyl, a phenylsulfonyl substituted by (Z)ₚ₁, a C₁ to C₆ alkylamino, a di(C₁ to C₆ alkyl)amino, phenylamino, a phenylamino substituted by (Z)ₚ₁, a C₁ to C₆ alkoxycarbonyl, phenyl, a phenyl substituted by (Z)ₚ₁, L-1 to L-5, L-8 to L-24, L-36, L-39, L-45 to L-52 or L-53,
L-1 to L-5, L-8 to L-24, L-36, L-39, L-45 to L-52 or L-53 each represent the following aromatic heterocyclic ring, 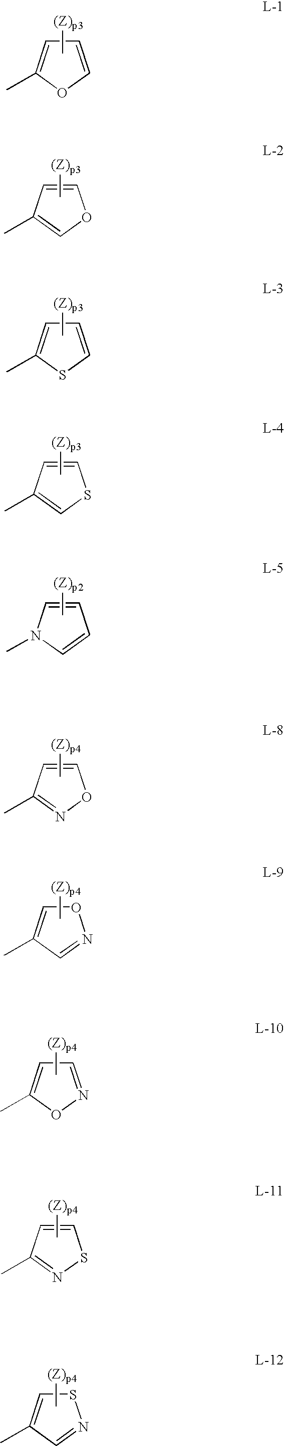 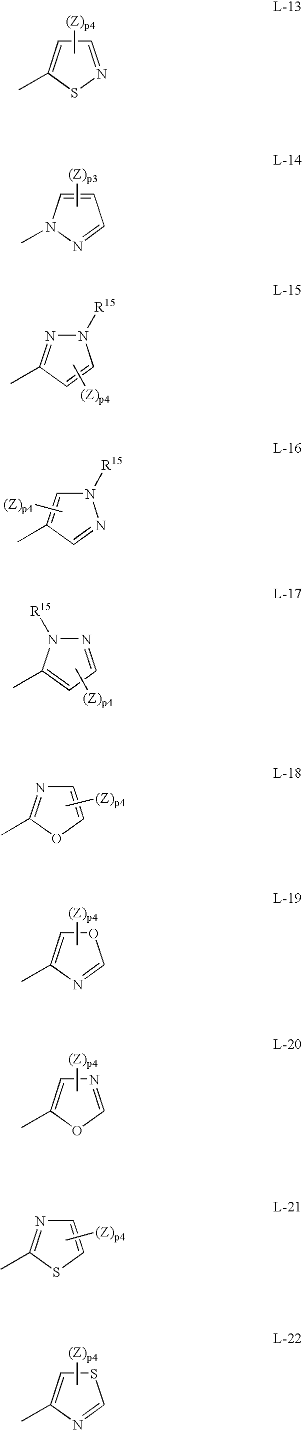 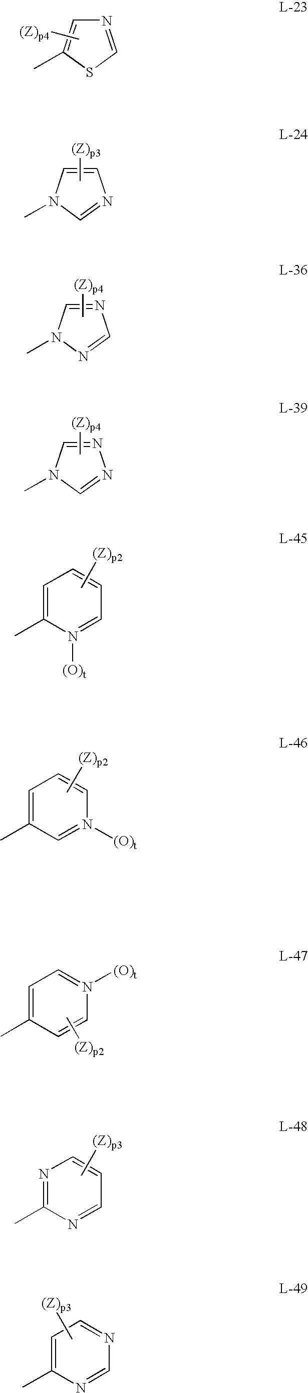 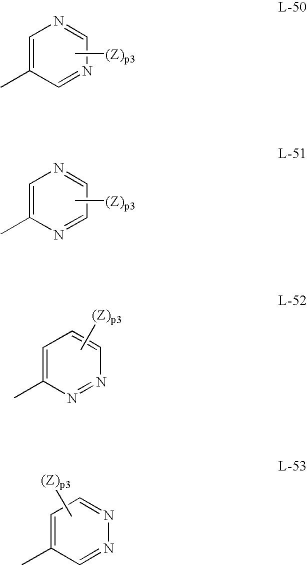
Z represents a halogen atom, cyano, nitro, a C₁ to C₆ alkyl, a C₁ to C₆ haloalkyl, a C₁ to C₃ alkylthio(C₁ to C₃) alkyl, a C₁ to C₃ haloalkylthio(C₁ to C₃) alkyl, a C₁ to C₃ alkylsulfinyl(C₁ to C₃) alkyl, a C₁ to C₃ haloalkylsulfinyl(C₁ to C₃) alkyl, a C₁ to C₃ alkylsulfonyl(C₁ to C₃) alkyl, a C₁ to C₃ haloalkylsulfonyl(C₁ to C₃) alkyl, a C₁ to C₆ alkoxy, a C₁ to C₆ haloalkoxy, a C₁ to C₃ haloalkoxy(C₁ to C₃) haloalkoxy, a C₁ to C₆ alkylsulfonyloxy, a C₁ to C₆ haloalkylsulfonyloxy, a C₁ to C₆ alkylthio, a C₁ to C₆ haloalkylthio, a C₁ to C₆ alkylsulfinyl, a C₁ to C₆ haloalkylsulfinyl, a C₁ to C₆ alkylsulfonyl, a C₁ to C₆ haloalkylsulfonyl, —C(O)NH₂ or —C(S)NH₂, when p1, p2, p3 or p4 is an integer of 2 or more, each Z may be the same or different from each other, further, when two Zs are adjacent to each other, the adjacent two Zs may form a 5-membered ring or 6-membered ring with the carbon atoms to which two Zs are bonded by forming —CF₂CF₂O—, —CF₂OCF₂—, —OCF₂O—, —OCF₂CHFO—, —OCF₂CF₂O— or —CH═CHCH═CH—,
R¹⁵ represents a C₁ to C₆ alkyl, phenyl or a phenyl substituted by (Z)ₚ₁,
p1 is an integer of 1 to 5,
p2 is an integer of 0 to 4,
p3 is an integer of 0 to 3,
p4 is an integer of 0 to 2,
p5 is an integer of 0 or 1,
r is an integer of 0 to 2,
t is an integer of 0 or 1.]
or a salt thereof.

6. A noxious organism controlling agent which comprises one or more kinds selected from the substituted benzanilide compound and a salt thereof according to claim 1 as an effective ingredient.

7. An agricultural chemical which comprises one or more kinds selected from the substituted benzanilide compound and a salt thereof according to claim 1 as an effective ingredient.

8. An insecticide or araricide which comprises one or more kinds selected from the substituted benzanilide compound and a salt thereof according to claim 1 as an effective ingredient.
